(19) United States
(12) Patent Application Publication
(10) Pub. No.: US 2005/0244935 A1
(43) Pub. Date: Nov. 3, 2005

(54) Corynebacterium glutamicum genes encoding proteins involved in membrane synthesis and membrane transport

(75) Inventors: Markus Pompejus, Freinsheim (DE); Burkhard Kroger, Limburgerhof (DE); Hartwig Schroder, Nussloch (DE); Oskar Zelder, Speyer (DE); Gregor Haberhauer, Limburgerhof (DE)

Correspondence Address: LAHIVE & COCKFIELD, LLP., 28 STATE STREET, BOSTON, MA, 02109, US

(73) Assignee: BASF AG, Ludwigshafen, DE (US)

(21) Appl. No.: 11/082,389

(22) Filed: Mar. 16, 2005

Related U.S. Application Data
(63) Continuation of application No. 09/603,024, filed on Jun. 23, 2000.
(60) Provisional application No. 60/141,031, filed on Jun. 25, 1999.
(60) Provisional application No. 60/143,262, filed on Jul. 9, 1999.
(60) Provisional application No. 60/151,281, filed on Aug. 27, 1999.

(30) Foreign Application Priority Data
Jul. 1, 1999 (DE) 19930487.4
Jul. 1, 1999 (DE) 19930489.0
Jul. 8, 1999 (DE) 19931549.3
Jul. 8, 1999 (DE) 19931550.7
Jul. 9, 1999 (DE) 19932134.5
Aug. 31, 1999 (DE) 19941379.7
Sep. 3, 1999 (DE) 19942088.2
Sep. 3, 1999 (DE) 19942097.1

(51) Int. Cl. C12P 13/04; C07H 21/04; C12N 9/10; C12N 1/21

(52) U.S. Cl. 435/106; 435/193; 435/252.3; 536/23.2

(57) ABSTRACT

Isolated nucleic acid molecules, designated MCT nucleic acid molecules, which encode novel MCT proteins from Corynebacterium glutamicum are described. The invention also provides antisense nucleic acid molecules, recombinant expression vectors containing MCT nucleic acid molecules, and host cells into which the expression vectors have been introduced. The invention still further provides isolated MCT proteins, mutated MCT proteins, fusion proteins, antigenic peptides and methods for the improvement of production of a desired compound from C. glutamicum based on genetic engineering of MCT genes in this organism.

### RELATED APPLICATIONS

This application is a continuation of U.S. patent application Ser. No. 09/603,024, filed Jun. 23, 2000, which claims priority to prior filed U.S. Provisional Patent Application Ser. No. 60/141,031, filed Jun. 25, 1999, U.S. Provisional Patent Application Ser. No. 60/143,262, filed Jul. 9, 1999, U.S. Provisional Patent Application Ser. No. 60/151,281, filed Aug. 27, 1999, German Patent Application No. 19930487.4, filed Jul. 1, 1999, German Patent Application No. 19930489.0, filed Jul. 1, 1999, German Patent Application No. 19931549.3, filed Jul. 8, 1999, German Patent Application No. 19931550.7, filed Jul. 8, 1999, German Patent Application No. 19932134.5, filed Jul. 9, 1999, German Patent Application No. 19941379.7, filed Aug. 31, 1999, German Patent Application No. 19942088.2, filed Sep. 3, 1999, and German Patent Application No. 19942097.1, filed Sep. 3, 1999. The entire contents of all of the aforementioned applications are hereby expressly incorporated herein by this reference.

### BACKGROUND OF THE INVENTION

Certain products and by-products of naturally-occurring metabolic processes in cells have utility in a wide array of industries, including the food, feed, cosmetics, and pharmaceutical industries. These molecules, collectively termed ‘fine chemicals’, include organic acids, both proteinogenic and non-proteinogenic amino acids, nucleotides and nucleosides, lipids and fatty acids, diols, carbohydrates, aromatic compounds, vitamins and cofactors, and enzymes. Their production is most conveniently performed through the large-scale culture of bacteria developed to produce and secrete large quantities of one or more desired molecules. One particularly useful organism for this purpose is Corynebacterium glutamicum, a gram positive, nonpathogenic bacterium. Through strain selection, a number of mutant strains have been developed which produce an array of desirable compounds. However, selection of strains improved for the production of a particular molecule is a time-consuming and difficult process.

### SUMMARY OF THE INVENTION

The invention provides novel bacterial nucleic acid molecules which have a variety of uses. These uses include the identification of microorganisms which can be used to produce fine chemicals, the modulation of fine chemical production in C. glutamicum or related bacteria, the typing or identification of C. glutamicum or related bacteria, as reference points for mapping the C. glutamicum genome, and as markers for transformation. These novel nucleic acid molecules encode proteins, referred to herein as membrane construction and membrane transport (MCT) proteins.

C. glutamicum is a gram positive, aerobic bacterium which is commonly used in industry for the large-scale production of a variety of fine chemicals, and also for the degradation of hydrocarbons (such as in petroleum spills) and for the oxidation of terpenoids. The MCT nucleic acid molecules of the invention, therefore, can be used to identify microorganisms which can be used to produce fine chemicals, e.g., by fermentation processes. Modulation of the expression of the MCT nucleic acids of the invention, or modification of the sequence of the MCT nucleic acid molecules of the invention, can be used to modulate the production of one or more fine chemicals from a microorganism (e.g., to improve the yield or production of one or more fine chemicals from a Corynebacterium or Brevibacterium species).

The MCT nucleic acids of the invention may also be used to identify an organism as being Corynebacterium glutamicum or a close relative thereof, or to identify the presence of C. glutamicum or a relative thereof in a mixed population of microorganisms. The invention provides the nucleic acid sequences of a number of C. glutamicum genes; by probing the extracted genomic DNA of a culture of a unique or mixed population of microorganisms under stringent conditions with a probe spanning a region of a C. glutamicum gene which is unique to this organism, one can ascertain whether this organism is present. Although Corynebacterium glutamicum itself is nonpathogenic, it is related to species pathogenic in humans, such as Corynebacterium diphtheriae (the causative agent of diphtheria); the detection of such organisms is of significant clinical relevance.

The MCT nucleic acid molecules of the invention may also serve as reference points for mapping of the C. glutamicum genome, or of genomes of related organisms. Similarly, these molecules, or variants or portions thereof, may serve as markers for genetically engineered Corynebacterium or Brevibacterium species.

The MCT proteins encoded by the novel nucleic acid molecules of the invention are capable of, for example, performing a function involved in the metabolism (e.g., the biosynthesis or degradation) of compounds necessary for membrane biosynthesis, or of assisting in the transmembrane transport of one or more compounds either into or out of the cell. Given the availability of cloning vectors for use in Corynebacterium glutamicum, such as those disclosed in Sinskey et al., U.S. Pat. No. 4,649,119, and techniques for genetic manipulation of C. glutamicum and the related Brevibacterium species (e.g., lactofermentum) (Yoshihama et al, J. Bacteriol. 162: 591-597 (1985); Katsumata et al., J. Bacteriol. 159: 306-311 (1984); and Santamaria et al., J. Gen. Microbiol. 130: 2237-2246 (1984)), the nucleic acid molecules of the invention may be utilized in the genetic engineering of this organism to make it a better or more efficient producer of one or more fine chemicals. This improved production or efficiency of production of a fine chemical may be due to a direct effect of manipulation of a gene of the invention, or it may be due to an indirect effect of such manipulation.

There are a number of mechanisms by which the alteration of an MCT protein of the invention may directly affect the yield, production, and/or efficiency of production of a fine chemical from a C. glutamicum strain incorporating such an altered protein. Those MCT proteins involved in the export of fine chemical molecules from the cell may be increased in number or activity such that greater quantities of these compounds are secreted to the extracellular medium, from which they are more readily recovered. Similarly, those MCT proteins involved in the import of nutrients necessary for the biosynthesis of one or more fine chemicals (e.g., phosphate, sulfate, nitrogen compounds, etc.) may be increased in number or activity such that these precursors, cofactors, or intermediate compounds are increased in concentration within the cell. Further, fatty acids and lipids themselves are desirable fine chemicals; by optimizing the activity or increasing the number of one or more MCT proteins of the invention which participate in the biosynthesis of these compounds, or by impairing the activity of one or more MCT proteins which are involved in the degradation of these compounds, it may be possible to increase the yield, production, and/or efficiency of production of fatty acid and lipid molecules from C. glutamicum.

The mutagenesis of one or more MCT genes of the invention may also result in MCT proteins having altered activities which indirectly impact the production of one or more desired fine chemicals from C. glutamicum. For example, MCT proteins of the invention involved in the export of waste products may be increased in number or activity such that the normal metabolic wastes of the cell (possibly increased in quantity due to the overproduction of the desired fine chemical) are efficiently exported before they are able to damage nucleotides and proteins within the cell (which would decrease the viability of the cell) or to interfere with fine chemical biosynthetic pathways (which would decrease the yield, production, or efficiency of production of the desired fine chemical). Further, the relatively large intracellular quantities of the desired fine chemical may in itself be toxic to the cell, so by increasing the activity or number of transporters able to export this compound from the cell, one may increase the viability of the cell in culture, in turn leading to a greater number of cells in the culture producing the desired fine chemical. The MCT proteins of the invention may also be manipulated such that the relative amounts of different lipid and fatty acid molecules are produced. This may have a profound effect on the lipid composition of the membrane of the cell. Since each type of lipid has different physical properties, an alteration in the lipid composition of a membrane may significantly alter membrane fluidity. Changes in membrane fluidity can impact the transport of molecules across the membrane, as well as the integrity of the cell, both of which have a profound effect on the production of fine chemicals from C. glutamicum in large-scale fermentative culture.

The invention provides novel nucleic acid molecules which encode proteins, referred to herein as MCT proteins, which are capable of, for example, participating in the metabolism of compounds necessary for the construction of cellular membranes in C. glutamicum, or in the transport of molecules across these membranes. Nucleic acid molecules encoding an MCT protein are referred to herein as MCT nucleic acid molecules. In a preferred embodiment, the MCT protein participates in the metabolism of compounds necessary for the construction of cellular membranes in C. glutamicum, or in the transport of molecules across these membranes. Examples of such proteins include those encoded by the genes set forth in Table 1.

Accordingly, one aspect of the invention pertains to isolated nucleic acid molecules (e.g., cDNAs, DNAs, or RNAs) comprising a nucleotide sequence encoding an MCT protein or biologically active portions thereof, as well as nucleic acid fragments suitable as primers or hybridization probes for the detection or amplification of MCT-encoding nucleic acid (e.g., DNA or mRNA). In particularly preferred embodiments, the isolated nucleic acid molecule comprises one of the nucleotide sequences set forth in Appendix A or the coding region or a complement thereof of one of these nucleotide sequences. In other particularly preferred embodiments, the isolated nucleic acid molecule of the invention comprises a nucleotide sequence which hybridizes to or is at least about 50%, preferably at least about 60%, more preferably at least about 70%, 80% or 90%, and even more preferably at least about 95%, 96%, 97%, 98%, 99% or more homologous to a nucleotide sequence set forth in Appendix A, or a portion thereof. In other preferred embodiments, the isolated nucleic acid molecule encodes one of the amino acid sequences set forth in Appendix B. The preferred MCT proteins of the present invention also preferably possess at least one of the MCT activities described herein.

In another embodiment, the isolated nucleic acid molecule encodes a protein or portion thereof wherein the protein or portion thereof includes an amino acid sequence which is sufficiently homologous to an amino acid sequence of Appendix B, e.g., sufficiently homologous to an amino acid sequence of Appendix B such that the protein or portion thereof maintains an MCT activity. Preferably, the protein or portion thereof encoded by the nucleic acid molecule maintains the ability to participate in the metabolism of compounds necessary for the construction of cellular membranes in C. glutamicum, or in the transport of molecules across these membranes. In one embodiment, the protein encoded by the nucleic acid molecule is at least about 50%, preferably at least about 60%, and more preferably at least about 70%, 80%, or 90% and most preferably at least about 95%, 96%, 97%, 98%, or 99% or more homologous to an amino acid sequence of Appendix B (e.g., an entire amino acid sequence selected from those sequences set forth in Appendix B). In another preferred embodiment, the protein is a full length C. glutamicum protein which is substantially homologous to an entire amino acid sequence of Appendix B (encoded by an open reading frame shown in Appendix A).

In another preferred embodiment, the isolated nucleic acid molecule is derived from C. glutamicum and encodes a protein (e.g., an MCT fusion protein) which includes a biologically active domain which is at least about 50% or more homologous to one of the amino acid sequences of Appendix B and is able to participate in the metabolism of compounds necessary for the construction of cellular membranes in C. glutamicum, or in the transport of molecules across these membranes, or has one or more of the activities set forth in Table 1, and which also includes heterologous nucleic acid sequences encoding a heterologous polypeptide or regulatory regions.

In another embodiment, the isolated nucleic acid molecule is at least 15 nucleotides in length and hybridizes under stringent conditions to a nucleic acid molecule comprising a nucleotide sequence of Appendix A. Preferably, the isolated nucleic acid molecule corresponds to a naturally-occurring nucleic acid molecule. More preferably, the isolated nucleic acid encodes a naturally-occurring C. glutamicum MCT protein, or a biologically active portion thereof.

Another aspect of the invention pertains to vectors, e.g., recombinant expression vectors, containing the nucleic acid molecules of the invention, and host cells into which such vectors have been introduced. In one embodiment, such a host cell is used to produce an MCT protein by culturing the host cell in a suitable medium. The MCT protein can be then isolated from the medium or the host cell.

Yet another aspect of the invention pertains to a genetically altered microorganism in which an MCT gene has been introduced or altered. In one embodiment, the genome of the microorganism has been altered by introduction of a nucleic acid molecule of the invention encoding wild-type or mutated MCT sequence as a transgene. In another embodiment, an endogenous MCT gene within the genome of the microorganism has been altered, e.g., functionally disrupted, by homologous recombination with an altered MCT gene. In another embodiment, an endogenous or introduced MCT gene in a microorganism has been altered by one or more point mutations, deletions, or inversions, but still encodes a functional MCT protein. In still another embodiment, one or more of the regulatory regions (e.g., a promoter, repressor, or inducer) of an MCT gene in a microorganism has been altered (e.g., by deletion, truncation, inversion, or point mutation) such that the expression of the MCT gene is modulated. In a preferred embodiment, the microorganism belongs to the genus Corynebacterium or Brevibacterium, with Corynebacterium glutamicum being particularly preferred. In a preferred embodiment, the microorganism is also utilized for the production of a desired compound, such as an amino acid, with lysine being particularly preferred.

In another aspect, the invention provides a method of identifying the presence or activity of Cornyebacterium diphtheriae in a subject. This method includes detection of one or more of the nucleic acid or amino acid sequences of the invention (e.g., the sequences set forth in Appendix A or Appendix B) in a subject, thereby detecting the presence or activity of Corynebacterium diphtheriae in the subject.

Still another aspect of the invention pertains to an isolated MCT protein or a portion, e.g., a biologically active portion, thereof. In a preferred embodiment, the isolated MCT protein or portion thereof can participate in the metabolism of compounds necessary for the construction of cellular membranes in C. glutamicum, or in the transport of molecules across these membranes. In another preferred embodiment, the isolated MCT protein or portion thereof is sufficiently homologous to an amino acid sequence of Appendix B such that the protein or portion thereof maintains the ability to participate in the metabolism of compounds necessary for the construction of cellular membranes in C. glutamicum, or in the transport of molecules across these membranes.

The invention also provides an isolated preparation of an MCT protein. In preferred embodiments, the MCT protein comprises an amino acid sequence of Appendix B. In another preferred embodiment, the invention pertains to an isolated full length protein which is substantially homologous to an entire amino acid sequence of Appendix B (encoded by an open reading frame set forth in Appendix A). In yet another embodiment, the protein is at least about 50%, preferably at least about 60%, and more preferably at least about 70%, 80%, or 90%, and most preferably at least about 95%, 96%, 97%, 98%, or 99% or more homologous to an entire amino acid sequence of Appendix B. In other embodiments, the isolated MCT protein comprises an amino acid sequence which is at least about 50% or more homologous to one of the amino acid sequences of Appendix B and is able to participate in the metabolism of compounds necessary for the construction of cellular membranes in C. glutamicum, or in the transport of molecules across these membranes, or has one or more of the activities set forth in Table 1.

Alternatively, the isolated MCT protein can comprise an amino acid sequence which is encoded by a nucleotide sequence which hybridizes, e.g., hybridizes under stringent conditions, or is at least about 50%, preferably at least about 60%, more preferably at least about 70%, 80%, or 90%, and even more preferably at least about 95%, 96%, 97%, 98,%, or 99% or more homologous, to a nucleotide sequence of Appendix B. It is also preferred that the preferred forms of MCT proteins also have one or more of the MCT bioactivities described herein.

The MCT polypeptide, or a biologically active portion thereof, can be operatively linked to a non-MCT polypeptide to form a fusion protein. In preferred embodiments, this fusion protein has an activity which differs from that of the MCT protein alone. In other preferred embodiments, this fusion protein participate in the metabolism of compounds necessary for the construction of cellular membranes in C. glutamicum, or in the transport of molecules across these membranes. In particularly preferred embodiments, integration of this fusion protein into a host cell modulates production of a desired compound from the cell.

In another aspect, the invention provides methods for screening molecules which modulate the activity of an MCT protein, either by interacting with the protein itself or a substrate or binding partner of the MCT protein, or by modulating the transcription or translation of an MCT nucleic acid molecule of the invention.

Another aspect of the invention pertains to a method for producing a fine chemical. This method involves the culturing of a cell containing a vector directing the expression of an MCT nucleic acid molecule of the invention, such that a fine chemical is produced. In a preferred embodiment, this method further includes the step of obtaining a cell containing such a vector, in which a cell is transfected with a vector directing the expression of an MCT nucleic acid. In another preferred embodiment, this method further includes the step of recovering the fine chemical from the culture. In a particularly preferred embodiment, the cell is from the genus Corynebacterium or Brevibacterium, or is selected from those strains set forth in Table 3.

Another aspect of the invention pertains to methods for modulating production of a molecule from a microorganism. Such methods include contacting the cell with an agent which modulates MCT protein activity or MCT nucleic acid expression such that a cell associated activity is altered relative to this same activity in the absence of the agent. In a preferred embodiment, the cell is modulated for one or more C. glutamicum metabolic pathways for cell membrane components or is modulated for the transport of compounds across such membranes, such that the yields or rate of production of a desired fine chemical by this microorganism is improved. The agent which modulates MCT protein activity can be an agent which stimulates MCT protein activity or MCT nucleic acid expression. Examples of agents which stimulate MCT protein activity or MCT nucleic acid expression include small molecules, active MCT proteins, and nucleic acids encoding MCT proteins that have been introduced into the cell. Examples of agents which inhibit MCT activity or expression include small molecules and antisense MCT nucleic acid molecules.

Another aspect of the invention pertains to methods for modulating yields of a desired compound from a cell, involving the introduction of a wild-type or mutant MCT gene into a cell, either maintained on a separate plasmid or integrated into the genome of the host cell. If integrated into the genome, such integration can be random, or it can take place by homologous recombination such that the native gene is replaced by the introduced copy, causing the production of the desired compound from the cell to be modulated. In a preferred embodiment, said yields are increased. In another preferred embodiment, said chemical is a fine chemical. In a particularly preferred embodiment, said fine chemical is an amino acid. In especially preferred embodiments, said amino acid is L-lysine.

### DETAILED DESCRIPTION OF THE INVENTION

The present invention provides MCT nucleic acid and protein molecules which are involved in the metabolism of cellular membrane components in C. glutamicum or in the transport of compounds across such membranes. The molecules of the invention may be utilized in the modulation of production of fine chemicals from microorganisms, such as C. glutamicum, either directly (e.g., where overexpression or optimization of a fatty acid biosynthesis protein has a direct impact on the yield, production, and/or efficiency of production of the fatty acid from modified C. glutamicum), or may have an indirect impact which nonetheless results in an increase of yield, production, and/or efficiency of production of the desired compound (e.g., where modulation of the metabolism of cell membrane components results in alterations in the yield, production, and/or efficiency of production or the composition of the cell membrane, which in turn may impact the production of one or more fine chemicals). Aspects of the invention are further explicated below.

I. Fine Chemicals

The term ‘fine chemical’ is art-recognized and includes molecules produced by an organism which have applications in various industries, such as, but not limited to, the pharmaceutical, agriculture, and cosmetics industries. Such compounds include organic acids, such as tartaric acid, itaconic acid, and diaminopimelic acid, both proteinogenic and non-proteinogenic amino acids, purine and pyrimidine bases, nucleosides, and nucleotides (as described e.g. in Kuninaka, A. (1996) Nucleotides and related compounds, p. 561-612, in Biotechnology vol. 6, Rehm et al., eds. VCH: Weinheim, and references contained therein), lipids, both saturated and unsaturated fatty acids (e.g., arachidonic acid), diols (e.g., propane diol, and butane diol), carbohydrates (e.g., hyaluronic acid and trehalose), aromatic compounds (e.g., aromatic amines, vanillin, and indigo), vitamins and cofactors (as described in Ullmann's Encyclopedia of Industrial Chemistry, vol. A27, “Vitamins”, p. 443-613 (1996) VCH: Weinheim and references therein; and Ong, A. S., Niki, E. & Packer, L. (1995) “Nutrition, Lipids, Health, and Disease” Proceedings of the UNESCO/Confederation of Scientific and Technological Associations in Malaysia, and the Society for Free Radical Research—Asia, held Sep. 1-3, 1994 at Penang, Malaysia, AOCS Press, (1995)), enzymes, polyketides (Cane et al. (1998) Science 282: 63-68), and all other chemicals described in Gutcho (1983) Chemicals by Fermentation, Noyes Data Corporation, ISBN: 0818805086 and references therein. The metabolism and uses of certain of these fine chemicals are further explicated below.

A. Amino Acid Metabolism and Uses

Amino acids comprise the basic structural units of all proteins, and as such are essential for normal cellular functioning in all organisms. The term “amino acid” is art-recognized. The proteinogenic amino acids, of which there are 20 species, serve as structural units for proteins, in which they are linked by peptide bonds, while the nonproteinogenic amino acids (hundreds of which are known) are not normally found in proteins (see Ulmann's Encyclopedia of Industrial Chemistry, vol. A2, p. 57-97 VCH: Weinheim (1985)). Amino acids may be in the D- or L-optical configuration, though L-amino acids are generally the only type found in naturally-occurring proteins. Biosynthetic and degradative pathways of each of the 20 proteinogenic amino acids have been well characterized in both prokaryotic and eukaryotic cells (see, for example, Stryer, L. Biochemistry, 3^{rd} edition, pages 578-590 (1988)). The ‘essential’ amino acids (histidine, isoleucine, leucine, lysine, methionine, phenylalanine, threonine, tryptophan, and valine), so named because they are generally a nutritional requirement due to the complexity of their biosyntheses, are readily converted by simple biosynthetic pathways to the remaining 11 ‘nonessential’ amino acids (alanine, arginine, asparagine, aspartate, cysteine, glutamate, glutamine, glycine, proline, serine, and tyrosine). Higher animals do retain the ability to synthesize some of these amino acids, but the essential amino acids must be supplied from the diet in order for normal protein synthesis to occur.

Aside from their function in protein biosynthesis, these amino acids are interesting chemicals in their own right, and many have been found to have various applications in the food, feed, chemical, cosmetics, agriculture, and pharmaceutical industries. Lysine is an important amino acid in the nutrition not only of humans, but also of monogastric animals such as poultry and swine. Glutamate is most commonly used as a flavor additive (mono-sodium glutamate, MSG) and is widely used throughout the food industry, as are aspartate, phenylalanine, glycine, and cysteine. Glycine, L-methionine and tryptophan are all utilized in the pharmaceutical industry. Glutamine, valine, leucine, isoleucine, histidine, arginine, pro line, serine and alanine are of use in both the pharmaceutical and cosmetics industries. Threonine, tryptophan, and D/L-methionine are common feed additives. (Leuchtenberger, W. (1996) Amino aids-technical production and use, p. 466-502 in Rehm et al. (eds.) Biotechnology vol. 6, chapter 14a, VCH: Weinheim). Additionally, these amino acids have been found to be useful as precursors for the synthesis of synthetic amino acids and proteins, such as N-acetylcysteine, S-carboxymethyl-L-cysteine, (S)-5-hydroxytryptophan, and others described in Ulmann's Encyclopedia of Industrial Chemistry, vol. A2, p. 57-97, VCH: Weinheim, 1985.

The biosynthesis of these natural amino acids in organisms capable of producing them, such as bacteria, has been well characterized (for review of bacterial amino acid biosynthesis and regulation thereof, see Umbarger, H. E. (1978) Ann. Rev. Biochem. 47: 533-606). Glutamate is synthesized by the reductive amination of α-ketoglutarate, an intermediate in the citric acid cycle. Glutamine, proline, and arginine are each subsequently produced from glutamate. The biosynthesis of serine is a three-step process beginning with 3-phosphoglycerate (an intermediate in glycolysis), and resulting in this amino acid after oxidation, transamination, and hydrolysis steps. Both cysteine and glycine are produced from serine; the former by the condensation of homocysteine with serine, and the latter by the transferal of the side-chain β-carbon atom to tetrahydrofolate, in a reaction catalyzed by serine transhydroxymethylase. Phenylalanine, and tyrosine are synthesized from the glycolytic and pentose phosphate pathway precursors erythrose 4-phosphate and phosphoenolpyruvate in a 9-step biosynthetic pathway that differ only at the final two steps after synthesis of prephenate. Tryptophan is also produced from these two initial molecules, but its synthesis is an 11-step pathway. Tyrosine may also be synthesized from phenylalanine, in a reaction catalyzed by phenylalanine hydroxylase. Alanine, valine, and leucine are all biosynthetic products of pyruvate, the final product of glycolysis. Aspartate is formed from oxaloacetate, an intermediate of the citric acid cycle. Asparagine, methionine, threonine, and lysine are each produced by the conversion of aspartate. Isoleucine is formed from threonine. A complex 9-step pathway results in the production of histidine from 5-phosphoribosyl-1-pyrophosphate, an activated sugar.

Amino acids in excess of the protein synthesis needs of the cell cannot be stored, and are instead degraded to provide intermediates for the major metabolic pathways of the cell (for review see Stryer, L. Biochemistry 3^{rd} ed. Ch. 21 “Amino Acid Degradation and the Urea Cycle” p. 495-516 (1988)). Although the cell is able to convert unwanted amino acids into useful metabolic intermediates, amino acid production is costly in terms of energy, precursor molecules, and the enzymes necessary to synthesize them. Thus it is not surprising that amino acid biosynthesis is regulated by feedback inhibition, in which the presence of a particular amino acid serves to slow or entirely stop its own production (for overview of feedback mechanisms in amino acid biosynthetic pathways, see Stryer, L. Biochemistry, 3^{rd} ed. Ch. 24: “Biosynthesis of Amino Acids and Heme” p. 575-600 (1988)). Thus, the output of any particular amino acid is limited by the amount of that amino acid present in the cell.

B. Vitamin, Cofactor, and Nutraceutical Metabolism and Uses

Vitamins, cofactors, and nutraceuticals comprise another group of molecules which the higher animals have lost the ability to synthesize and so must ingest, although they are readily synthesized by other organisms such as bacteria. These molecules are either bioactive substances themselves, or are precursors of biologically active substances which may serve as electron carriers or intermediates in a variety of metabolic pathways. Aside from their nutritive value, these compounds also have significant industrial value as coloring agents, antioxidants, and catalysts or other processing aids. (For an overview of the structure, activity, and industrial applications of these compounds, see, for example, Ullman's Encyclopedia of Industrial Chemistry, “Vitamins” vol. A27, p. 443-613, VCH: Weinheim, 1996.) The term “vitamin” is art-recognized, and includes nutrients which are required by an organism for normal functioning, but which that organism cannot synthesize by itself. The group of vitamins may encompass cofactors and nutraceutical compounds. The language “cofactor” includes nonproteinaceous compounds required for a normal enzymatic activity to occur. Such compounds may be organic or inorganic; the cofactor molecules of the invention are preferably organic. The term “nutraceutical” includes dietary supplements having health benefits in plants and animals, particularly humans. Examples of such molecules are vitamins, antioxidants, and also certain lipids (e.g., polyunsaturated fatty acids).

The biosynthesis of these molecules in organisms capable of producing them, such as bacteria, has been largely characterized (Ullman's Encyclopedia of Industrial Chemistry, “Vitamins” vol. A27, p. 443-613, VCH: Weinheim, 1996; Michal, G. (1999) Biochemical Pathways: An Atlas of Biochemistry and Molecular Biology, John Wiley & Sons; Ong, A. S., Niki, E. & Packer, L. (1995) “Nutrition, Lipids, Health, and Disease” Proceedings of the UNESCO/Confederation of Scientific and Technological Associations in Malaysia, and the Society for Free Radical Research—Asia, held Sep. 1-3, 1994 at Penang, Malaysia, AOCS Press: Champaign, Ill. X, 374 S).

Thiamin (vitamin B₁) is produced by the chemical coupling of pyrimidine and thiazole moieties. Riboflavin (vitamin B₂) is synthesized from guanosine-5′-triphosphate (GTP) and ribose-5′-phosphate. Riboflavin, in turn, is utilized for the synthesis of flavin mononucleotide (FMN) and flavin adenine dinucleotide (FAD). The family of compounds collectively termed ‘vitamin B₆’ (e.g., pyridoxine, pyridoxamine, pyridoxa-5′-phosphate, and the commercially used pyridoxin hydrochloride) are all derivatives of the common structural unit, 5-hydroxy-6-methylpyridine. Pantothenate (pantothenic acid, (R)-(+)-N-(2,4-dihydroxy-3,3-dimethyl-1-oxobutyl)-β-alanine) can be produced either by chemical synthesis or by fermentation. The final steps in pantothenate biosynthesis consist of the ATP-driven condensation of β-alanine and pantoic acid. The enzymes responsible for the biosynthesis steps for the conversion to pantoic acid, to β-alanine and for the condensation to panthotenic acid are known. The metabolically active form of pantothenate is Coenzyme A, for which the biosynthesis proceeds in 5 enzymatic steps. Pantothenate, pyridoxal-5′-phosphate, cysteine and ATP are the precursors of Coenzyme A. These enzymes not only catalyze the formation of panthothante, but also the production of (R)-pantoic acid, (R)-pantolacton, (R)-panthenol (provitamin B₅), pantetheine (and its derivatives) and coenzyme A.

Biotin biosynthesis from the precursor molecule pimeloyl-CoA in microorganisms has been studied in detail and several of the genes involved have been identified. Many of the corresponding proteins have been found to also be involved in Fe-cluster synthesis and are members of the nifS class of proteins. Lipoic acid is derived from octanoic acid, and serves as a coenzyme in energy metabolism, where it becomes part of the pyruvate dehydrogenase complex and the α-ketoglutarate dehydrogenase complex. The folates are a group of substances which are all derivatives of folic acid, which is turn is derived from L-glutamic acid, p-amino-benzoic acid and 6-methylpterin. The biosynthesis of folic acid and its derivatives, starting from the metabolism intermediates guanosine-5′-triphosphate (GTP), L-glutamic acid and p-amino-benzoic acid has been studied in detail in certain microorganisms.

Corrinoids (such as the cobalamines and particularly vitamin B₁₂) and porphyrines belong to a group of chemicals characterized by a tetrapyrole ring system. The biosynthesis of vitamin B₁₂ is sufficiently complex that it has not yet been completely characterized, but many of the enzymes and substrates involved are now known. Nicotinic acid (nicotinate), and nicotinamide are pyridine derivatives which are also termed ‘niacin’. Niacin is the precursor of the important coenzymes NAD (nicotinamide adenine dinucleotide) and NADP (nicotinamide adenine dinucleotide phosphate) and their reduced forms.

The large-scale production of these compounds has largely relied on cell-free chemical syntheses, though some of these chemicals have also been produced by large-scale culture of microorganisms, such as riboflavin, Vitamin B₆, pantothenate, and biotin. Only Vitamin B₁₂ is produced solely by fermentation, due to the complexity of its synthesis. In vitro methodologies require significant inputs of materials and time, often at great cost.

C. Purine, Pyrimidine, Nucleoside and Nucleotide Metabolism and Uses

Purine and pyrimidine metabolism genes and their corresponding proteins are important targets for the therapy of tumor diseases and viral infections. The language “purine” or “pyrimidine” includes the nitrogenous bases which are constituents of nucleic acids, co-enzymes, and nucleotides. The term “nucleotide” includes the basic structural units of nucleic acid molecules, which are comprised of a nitrogenous base, a pentose sugar (in the case of RNA, the sugar is ribose; in the case of DNA, the sugar is D-deoxyribose), and phosphoric acid. The language “nucleoside” includes molecules which serve as precursors to nucleotides, but which are lacking the phosphoric acid moiety that nucleotides possess. By inhibiting the biosynthesis of these molecules, or their mobilization to form nucleic acid molecules, it is possible to inhibit RNA and DNA synthesis; by inhibiting this activity in a fashion targeted to cancerous cells, the ability of tumor cells to divide and replicate may be inhibited. Additionally, there are nucleotides which do not form nucleic acid molecules, but rather serve as energy stores (i.e., AMP) or as coenzymes (i.e., FAD and NAD).

Several publications have described the use of these chemicals for these medical indications, by influencing purine and/or pyrimidine metabolism (e.g. Christopherson, R. I., and Lyons, S. D. (1990) “Potent inhibitors of de novo pyrimidine and purine biosynthesis as chemotherapeutic agents.” Med. Res. Reviews 10: 505-548). Studies of enzymes involved in purine and pyrimidine metabolism have been focused on the development of new drugs which can be used, for example, as immunosuppressants or anti-proliferants (Smith, J. L., (1995) “Enzymes in nucleotide synthesis.” Curr. Opin. Struct. Biol. 5: 752-757; (1995) Biochem Soc. Transact. 23: 877-902). However, purine and pyrimidine bases, nucleosides and nucleotides have other utilities: as intermediates in the biosynthesis of several fine chemicals (e.g., thiamine, S-adenosyl-methionine, folates, or riboflavin), as energy carriers for the cell (e.g., ATP or GTP), and for chemicals themselves, commonly used as flavor enhancers (e.g., IMP or GMP) or for several medicinal applications (see, for example, Kuninaka, A. (1996) Nucleotides and Related Compounds in Biotechnology vol. 6, Rehm et al., eds. VCH: Weinheim, p. 561-612). Also, enzymes involved in purine, pyrimidine, nucleoside, or nucleotide metabolism are increasingly serving as targets against which chemicals for crop protection, including fungicides, herbicides and insecticides, are developed.

The metabolism of these compounds in bacteria has been characterized (for reviews see, for example, Zalkin, H. and Dixon, J. E. (1992) “de novo purine nucleotide biosynthesis”, in: Progress in Nucleic Acid Research and Molecular Biology, vol. 42, Academic Press: p. 259-287; and Michal, G. (1999) “Nucleotides and Nucleosides”, Chapter 8 in: Biochemical Pathways: An Atlas of Biochemistry and Molecular Biology, Wiley: New York). Purine metabolism has been the subject of intensive research, and is essential to the normal functioning of the cell. Impaired purine metabolism in higher animals can cause severe disease, such as gout. Purine nucleotides are synthesized from ribose-5-phosphate, in a series of steps through the intermediate compound inosine-5′-phosphate (IMP), resulting in the production of guanosine-5′-monophosphate (GMP) or adenosine-5′-monophosphate (AMP), from which the triphosphate forms utilized as nucleotides are readily formed. These compounds are also utilized as energy stores, so their degradation provides energy for many different biochemical processes in the cell. Pyrimidine biosynthesis proceeds by the formation of uridine-5′-monophosphate (UMP) from ribose-5-phosphate. UMP, in turn, is converted to cytidine-5′-triphosphate (CTP). The deoxy-forms of all of these nucleotides are produced in a one step reduction reaction from the diphosphate ribose form of the nucleotide to the diphosphate deoxyribose form of the nucleotide. Upon phosphorylation, these molecules are able to participate in DNA synthesis.

D. Trehalose Metabolism and Uses

Trehalose consists of two glucose molecules, bound in α, α-1,1 linkage. It is commonly used in the food industry as a sweetener, an additive for dried or frozen foods, and in beverages. However, it also has applications in the pharmaceutical, cosmetics and biotechnology industries (see, for example, Nishimoto et al., (1998) U.S. Pat. No. 5,759,610; Singer, M. A. and Lindquist, S. (1998) Trends Biotech. 16: 460-467; Paiva, C. L. A. and Panek, A. D. (1996) Biotech. Ann. Rev. 2: 293-314; and Shiosaka, M. (1997) J. Japan 172: 97-102). Trehalose is produced by enzymes from many microorganisms and is naturally released into the surrounding medium, from which it can be collected using methods known in the art.

II. Membrane Biosynthesis and Transmembrane Transport

Cellular membranes serve a variety of functions in a cell. First and foremost, a membrane differentiates the contents of a cell from the surrounding environment, thus giving integrity to the cell. Membranes may also serve as barriers to the influx of hazardous or unwanted compounds, and also to the efflux of desired compounds. Cellular membranes are by nature impervious to the unfacilitated diffusion of hydrophilic compounds such as proteins, water molecules and ions due to their structure: a bilayer of lipid molecules in which the polar head groups face outwards (towards the exterior and interior of the cell, respectively) and the nonpolar tails face inwards at the center of the bilayer, forming a hydrophobic core (for a general review of membrane structure and function, see Gennis, R. B. (1989) Biomembranes, Molecular Structure and Function, Springer: Heidelberg). This barrier enables cells to maintain a relatively higher concentration of desired compounds and a relatively lower concentration of undesired compounds than are contained within the surrounding medium, since the diffusion of these compounds is effectively blocked by the membrane. However, the membrane also presents an effective barrier to the import of desired compounds and the export of waste molecules. To overcome this difficulty, cellular membranes incorporate many kinds of transporter proteins which are able to facilitate the transmembrane transport of different kinds of compounds. There are two general classes of these transport proteins: pores or channels and transporters. The former are integral membrane proteins, sometimes complexes of proteins, which form a regulated hole through the membrane. This regulation, or ‘gating’ is generally specific to the molecules to be transported by the pore or channel, rendering these transmembrane constructs selectively permeable to a specific class of substrates; for example, a potassium channel is constructed such that only ions having a like charge and size to that of potassium may pass through. Channel and pore proteins tend to have discrete hydrophobic and hydrophilic domains, such that the hydrophobic face of the protein may associate with the interior of the membrane while the hydrophilic face lines the interior of the channel, thus providing a sheltered hydrophilic environment through which the selected hydrophilic molecule may pass. Many such pores/channels are known in the art, including those for potassium, calcium, sodium, and chloride ions.

This pore and channel-mediated system of facilitated diffusion is limited to very small molecules, such as ions, because pores or channels large enough to permit the passage of whole proteins by facilitated diffusion would be unable to prevent the passage of smaller hydrophilic molecules as well. Transport of molecules by this process is sometimes termed ‘facilitated diffusion’ since the driving force of a concentration gradient is required for the transport to occur. Permeases also permit facilitated diffusion of larger molecules, such as glucose or other sugars, into the cell when the concentration of these molecules on one side of the membrane is greater than that on the other (also called ‘uniport’). In contrast to pores or channels, these integral membrane proteins (often having between 6-14 membrane-spanning α-helices) do not form open channels through the membrane, but rather bind to the target molecule at the surface of the membrane and then undergo a conformational shift such that the target molecule is released on the opposite side of the membrane.

However, cells frequently require the import or export of molecules against the existing concentration gradient (‘active transport’), a situation in which facilitated diffusion cannot occur. There are two general mechanisms used by cells for such membrane transport: symport or antiport, and energy-coupled transport such as that mediated by the ABC transporters. Symport and antiport systems couple the movement of two different molecules across the membrane (via permeases having two separate binding sites for the two different molecules); in symport, both molecules are transported in the same direction, while in antiport, one molecule is imported while the other is exported. This is possible energetically because one of the two molecules moves in accordance with a concentration gradient, and this energetically favorable event is permitted only upon concomitant movement of a desired compound against the prevailing concentration gradient. Single molecules may be transported across the membrane against the concentration gradient in an energy-driven process, such as that utilized by the ABC transporters. In this system, the transport protein located in the membrane has an ATP-binding cassette; upon binding of the target molecule, the ATP is converted to ADP+Pi, and the resulting release of energy is used to drive the movement of the target molecule to the opposite face of the membrane, facilitated by the transporter. For more detailed descriptions of all of these transport systems, see: Bamberg, E. et al., (1993) “Charge transport of ion pumps on lipid bilayer membranes”, Q. Rev. Biophys. 26: 1-25; Findlay, J. B. C. (1991) “Structure and function in membrane transport systems”, Curr. Opin. Struct. Biol. 1: 804-810; Higgins, C. F. (1992) “ABC transporters from microorganisms to man”, Ann. Rev. Cell Biol. 8: 67-113; Gennis, R. B. (1989) “Pores, Channels and Transporters”, in: Biomembranes, Molecular Structure and Function, Springer: Heidelberg, p. 270-322; and Nikaido, H. and Saier, H. (1992) “Transport proteins in bacteria: common themes in their design”, Science 258: 936-942, and references contained within each of these references.

The synthesis of membranes is a well-characterized process involving a number of components, the most important of which are lipid molecules. Lipid synthesis may be divided into two parts: the synthesis of fatty acids and their attachment to sn-glycerol-3-phosphate, and the addition or modification of a polar head group. Typical lipids utilized in bacterial membranes include phospholipids, glycolipids, sphingolipids, and phosphoglycerides. Fatty acid synthesis begins with the conversion of acetyl CoA either to malonyl CoA by acetyl CoA carboxylase, or to acetyl-ACP by acetyltransacylase. Following a condensation reaction, these two product molecules together form acetoacetyl-ACP, which is converted by a series of condensation, reduction and dehydration reactions to yield a saturated fatty acid molecule having a desired chain length. The production of unsaturated fatty acids from such molecules is catalyzed by specific desaturases either aerobically, with the help of molecular oxygen, or anaerobically (for reference on fatty acid synthesis, see F. C. Neidhardt et al. (1996) E. coli and Salmonella. ASM Press: Washington, D.C., p. 612-636 and references contained therein; Lengeler et al. (eds) (1999) Biology of Procaryotes. Thieme: Stuttgart, N.Y., and references contained therein; and Magnuson, K. et al., (1993) Microbiological Reviews 57: 522-542, and references contained therein). The cyclopropane fatty acids (CFA) are synthesized by a specific CFA-synthase using SAM as a cosubstrate. Branched chain fatty acids are synthesized from branched chain amino acids that are deaminated to yield branched chain 2-oxo-acids (see Lengeler et al., eds. (1999) Biology of Procaryotes. Thieme: Stuttgart, N.Y., and references contained therein). Another essential step in lipid synthesis is the transfer of fatty acids onto the polar head groups by, for example, glycerol-phosphate-acyltransferases. The combination of various precursor molecules and biosynthetic enzymes results in the production of different fatty acid molecules, which has a profound effect on the composition of the membrane.

III. Elements and Methods of the Invention

The present invention is based, at least in part, on the discovery of novel molecules, referred to herein as MCT nucleic acid and protein molecules, which control the production of cellular membranes in C. glutamicum and govern the movement of molecules across such membranes. In one embodiment, the MCT molecules participate in the metabolism of compounds necessary for the construction of cellular membranes in C. glutamicum, or in the transport of molecules across these membranes. In a preferred embodiment, the activity of the MCT molecules of the present invention to regulate membrane component production and membrane transport has an impact on the production of a desired fine chemical by this organism. In a particularly preferred embodiment, the MCT molecules of the invention are modulated in activity, such that the C. glutamicum metabolic pathways which the MCT proteins of the invention regulate are modulated in yield, production, and/or efficiency of production and the transport of compounds through the membranes is altered in efficiency, which either directly or indirectly modulates the yield, production, and/or efficiency of production of a desired fine chemical by C. glutamicum.

The language, “MCT protein” or “MCT polypeptide” includes proteins which participate in the metabolism of compounds necessary for the construction of cellular membranes in C. glutamicum, or in the transport of molecules across these membranes. Examples of MCT proteins include those encoded by the MCT genes set forth in Table 1 and Appendix A. The terms “MCT gene” or “MCT nucleic acid sequence” include nucleic acid sequences encoding an MCT protein, which consist of a coding region and also corresponding untranslated 5′ and 3′ sequence regions. Examples of MCT genes include those set forth in Table 1. The terms “production” or “productivity” are art-recognized and include the concentration of the fermentation product (for example, the desired fine chemical) formed within a given time and a given fermentation volume (e.g., kg product per hour per liter). The term “efficiency of production” includes the time required for a particular level of production to be achieved (for example, how long it takes for the cell to attain a particular rate of output of a fine chemical). The term “yield” or “product/carbon yield” is art-recognized and includes the efficiency of the conversion of the carbon source into the product (i.e., fine chemical). This is generally written as, for example, kg product per kg carbon source. By increasing the yield or production of the compound, the quantity of recovered molecules, or of useful recovered molecules of that compound in a given amount of culture over a given amount of time is increased. The terms “biosynthesis” or a “biosynthetic pathway” are art-recognized and include the synthesis of a compound, preferably an organic compound, by a cell from intermediate compounds in what may be a multistep and highly regulated process. The terms “degradation” or a “degradation pathway” are art-recognized and include the breakdown of a compound, preferably an organic compound, by a cell to degradation products (generally speaking, smaller or less complex molecules) in what may be a multistep and highly regulated process. The language “metabolism” is art-recognized and includes the totality of the biochemical reactions that take place in an organism. The metabolism of a particular compound, then, (e.g., the metabolism of an amino acid such as glycine) comprises the overall biosynthetic, modification, and degradation pathways in the cell related to this compound.

In another embodiment, the MCT molecules of the invention are capable of modulating the production of a desired molecule, such as a fine chemical, in a microorganism such as C. glutamicum. There are a number of mechanisms by which the alteration of an MCT protein of the invention may directly affect the yield, production, and/or efficiency of production of a fine chemical from a C. glutamicum strain incorporating such an altered protein. Those MCT proteins involved in the export of fine chemical molecules from the cell may be increased in number or activity such that greater quantities of these compounds are secreted to the extracellular medium, from which they are more readily recovered. Similarly, those MCT proteins involved in the import of nutrients necessary for the biosynthesis of one or more fine chemicals (e.g., phosphate, sulfate, nitrogen compounds, etc.) may be increased in number or activity such that these precursor, cofactor, or intermediate compounds are increased in concentration within the cell. Further, fatty acids and lipids themselves are desirable fine chemicals; by optimizing the activity or increasing the number of one or more MCT proteins of the invention which participate in the biosynthesis of these compounds, or by impairing the activity of one or more MCT proteins which are involved in the degradation of these compounds, it may be possible to increase the yield, production, and/or efficiency of production of fatty acid and lipid molecules from C. glutamicum.

The mutagenesis of one or more MCT genes of the invention may also result in MCT proteins having altered activities which indirectly impact the production of one or more desired fine chemicals from C. glutamicum. For example, MCT proteins of the invention involved in the export of waste products may be increased in number or activity such that the normal metabolic wastes of the cell (possibly increased in quantity due to the overproduction of the desired fine chemical) are efficiently exported before they are able to damage nucleotides and proteins within the cell (which would decrease the viability of the cell) or to interfere with fine chemical biosynthetic pathways (which would decrease the yield, production, or efficiency of production of the desired fine chemical). Further, the relatively large intracellular quantities of the desired fine chemical may in itself be toxic to the cell, so by increasing the activity or number of transporters able to export this compound from the cell, one may increase the viability of the cell in culture, in turn leading to a greater number of cells in the culture producing the desired fine chemical. The MCT proteins of the invention may also be manipulated such that the relative amounts of different lipid and fatty acid molecules are produced. This may have a profound effect on the lipid composition of the membrane of the cell. Since each type of lipid has different physical properties, an alteration in the lipid composition of a membrane may significantly alter membrane fluidity. Changes in membrane fluidity can impact the transport of molecules across the membrane, as well as the integrity of the cell, both of which have a profound effect on the production of fine chemicals from C. glutamicum in large-scale fermentative culture.

The isolated nucleic acid sequences of the invention are contained within the genome of a Corynebacterium glutamicum strain available through the American Type Culture Collection, given designation ATCC 13032. The nucleotide sequence of the isolated C. glutamicum MCT DNAs and the predicted amino acid sequences of the C. glutamicum MCT proteins are shown in Appendices A and B, respectively. Computational analyses were performed which classified and/or identified these nucleotide sequences as sequences which encode proteins involved in the metabolism of cellular membrane components or proteins involved in the transport of compounds across such membranes.

The present invention also pertains to proteins which have an amino acid sequence which is substantially homologous to an amino acid sequence of Appendix B. As used herein, a protein which has an amino acid sequence which is substantially homologous to a selected amino acid sequence is least about 50% homologous to the selected amino acid sequence, e.g., the entire selected amino acid sequence. A protein which has an amino acid sequence which is substantially homologous to a selected amino acid sequence can also be least about 50-60%, preferably at least about 60-70%, and more preferably at least about 70-80%, 80-90%, or 90-95%, and most preferably at least about 96%, 97%, 98%, 99% or more homologous to the selected amino acid sequence.

The MCT protein or a biologically active portion or fragment thereof of the invention can participate in the metabolism of compounds necessary for the construction of cellular membranes in C. glutamicum, or in the transport of molecules across these membranes, or have one or more of the activities set forth in Table 1.

Various aspects of the invention are described in further detail in the following subsections:

A. Isolated Nucleic Acid Molecules

One aspect of the invention pertains to isolated nucleic acid molecules that encode MCT polypeptides or biologically active portions thereof, as well as nucleic acid fragments sufficient for use as hybridization probes or primers for the identification or amplification of MCT-encoding nucleic acid (e.g., MCT DNA). As used herein, the term “nucleic acid molecule” is intended to include DNA molecules (e.g., cDNA or genomic DNA) and RNA molecules (e.g., mRNA) and analogs of the DNA or RNA generated using nucleotide analogs. This term also encompasses untranslated sequence located at both the 3′ and 5′ ends of the coding region of the gene: at least about 100 nucleotides of sequence upstream from the 5′ end of the coding region and at least about 20 nucleotides of sequence downstream from the 3′end of the coding region of the gene. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated MCT nucleic acid molecule can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived (e.g, a C. glutamicum cell). Moreover, an “isolated” nucleic acid molecule, such as a DNA molecule, can be substantially free of other cellular material, or culture medium when produced by recombinant techniques, or chemical precursors or other chemicals when chemically synthesized.

A nucleic acid molecule of the present invention, e.g., a nucleic acid molecule having a nucleotide sequence of Appendix A, or a portion thereof, can be isolated using standard molecular biology techniques and the sequence information provided herein. For example, a C. glutamicum MCT DNA can be isolated from a C. glutamicum library using all or portion of one of the sequences of Appendix A as a hybridization probe and standard hybridization techniques (e.g., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Moreover, a nucleic acid molecule encompassing all or a portion of one of the sequences of Appendix A can be isolated by the polymerase chain reaction using oligonucleotide primers designed based upon this sequence (e.g., a nucleic acid molecule encompassing all or a portion of one of the sequences of Appendix A can be isolated by the polymerase chain reaction using oligonucleotide primers designed based upon this same sequence of Appendix A). For example, mRNA can be isolated from normal endothelial cells (e.g., by the guanidinium-thiocyanate extraction procedure of Chirgwin et al. (1979) Biochemistry 18: 5294-5299) and DNA can be prepared using reverse transcriptase (e.g., Moloney MLV reverse transcriptase, available from Gibco/BRL, Bethesda, Md.; or AMV reverse transcriptase, available from Seikagaku America, Inc., St. Petersburg, Fla.). Synthetic oligonucleotide primers for polymerase chain reaction amplification can be designed based upon one of the nucleotide sequences shown in Appendix A. A nucleic acid of the invention can be amplified using cDNA or, alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to an MCT nucleotide sequence can be prepared by standard synthetic techniques, e.g., using an automated DNA synthesizer.

In a preferred embodiment, an isolated nucleic acid molecule of the invention comprises one of the nucleotide sequences shown in Appendix A. The sequences of Appendix A correspond to the Corynebacterium glutamicum MCT DNAs of the invention. This DNA comprises sequences encoding MCT proteins (i.e., the “coding region”, indicated in each sequence in Appendix A), as well as 5′ untranslated sequences and 3′ untranslated sequences, also indicated in Appendix A. Alternatively, the nucleic acid molecule can comprise only the coding region of any of the sequences in Appendix A.

For the purposes of this application, it will be understood that each of the sequences set forth in Appendix A has an identifying RXA, RXN, or RXS number having the designation “RXA”, “RXN”, or “RXS” followed by 5 digits (i.e., RXA00775, RXN02994, or RXS03221). Each of these sequences comprises up to three parts: a 5′ upstream region, a coding region, and a downstream region. Each of these three regions is identified by the same RXA, RXN, or RXS designation to eliminate confusion. The recitation “one of the sequences in Appendix A”, then, refers to any of the sequences in Appendix A, which may be distinguished by their differing RXA, RXN, or RXS designations. The coding region of each of these sequences is translated into a corresponding amino acid sequence, which is set forth in Appendix B. The sequences of Appendix B are identified by the same RXA, RXN, or RXS designations as Appendix A, such that they can be readily correlated. For example, the amino acid sequences in Appendix B designated RXA00775, RXN02994, and RXS03221 are translations of the coding regions of the nucleotide sequence of nucleic acid molecules RXA00775, RXN02994, and RXS03221, respectively, in Appendix A. Each of the RXA, RXN, and RXS nucleotide and amino acid sequences of the invention has also been assigned a SEQ ID NO, as indicated in Table 1. For example, as set forth in Table 1, the nucleic acid sequence of RXA00774 is SEQ ID NO:7, and the amino acid sequence of RXA00774 is SEQ ID NO:8.

Several of the genes of the invention are “F-designated genes”. An F-designated gene includes those genes set forth in Table 1 which have an ‘F’ in front of the RXA, RXN, or RXS designation. For example, SEQ ID NO:21, designated, as indicated on Table 1, as “F RXA01245”, is an F-designated gene, as are SEQ ID NOs: 35, 39, and 43 (designated on Table 1 as “F RXA01164”, “F RXA01168”, and “F RXA02062”, respectively).

In one embodiment, the nucleic acid molecules of the present invention are not intended to include those compiled in Table 2. In the case of the dapD gene, a sequence for this gene was published in Wehrmann, A., et al. (1998) J. Bacteriol. 180 (12): 3159-3165. However, the sequence obtained by the inventors of the present application is significantly longer than the published version. It is believed that the published version relied on an incorrect start codon, and thus represents only a fragment of the actual coding region.

In another preferred embodiment, an isolated nucleic acid molecule of the invention comprises a nucleic acid molecule which is a complement of one of the nucleotide sequences shown in Appendix A, or a portion thereof. A nucleic acid molecule which is complementary to one of the nucleotide sequences shown in Appendix A is one which is sufficiently complementary to one of the nucleotide sequences shown in Appendix A such that it can hybridize to one of the nucleotide sequences shown in Appendix A, thereby forming a stable duplex.

In still another preferred embodiment, an isolated nucleic acid molecule of the invention comprises a nucleotide sequence which is at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more homologous to a nucleotide sequence shown in Appendix A, or a portion thereof. Ranges and identity values intermediate to the above-recited ranges, (e.g., 70-90% identical or 80-95% identical) are also intended to be encompassed by the present invention. For example, ranges of identity values using a combination of any of the above values recited as upper and/or lower limits are intended to be included. In an additional preferred embodiment, an isolated nucleic acid molecule of the invention comprises a nucleotide sequence which hybridizes, e.g., hybridizes under stringent conditions, to one of the nucleotide sequences shown in Appendix A, or a portion thereof.

Moreover, the nucleic acid molecule of the invention can comprise only a portion of the coding region of one of the sequences in Appendix A, for example a fragment which can be used as a probe or primer or a fragment encoding a biologically active portion of an MCT protein. The nucleotide sequences determined from the cloning of the MCT genes from C. glutamicum allows for the generation of probes and primers designed for use in identifying and/or cloning MCT homologues in other cell types and organisms, as well as MCT homologues from other Corynebacteria or related species. The probe/primer typically comprises substantially purified oligonucleotide. The oligonucleotide typically comprises a region of nucleotide sequence that hybridizes under stringent conditions to at least about 12, preferably about 25, more preferably about 40, 50 or 75 consecutive nucleotides of a sense strand of one of the sequences set forth in Appendix A, an anti-sense sequence of one of the sequences set forth in Appendix A, or naturally occurring mutants thereof. Primers based on a nucleotide sequence of Appendix A can be used in PCR reactions to clone MCT homologues. Probes based on the MCT nucleotide sequences can be used to detect transcripts or genomic sequences encoding the same or homologous proteins. In preferred embodiments, the probe further comprises a label group attached thereto, e.g. the label group can be a radioisotope, a fluorescent compound, an enzyme, or an enzyme co-factor. Such probes can be used as a part of a diagnostic test kit for identifying cells which misexpress an MCT protein, such as by measuring a level of an MCT-encoding nucleic acid in a sample of cells, e.g., detecting MCT mRNA levels or determining whether a genomic MCT gene has been mutated or deleted.

In one embodiment, the nucleic acid molecule of the invention encodes a protein or portion thereof which includes an amino acid sequence which is sufficiently homologous to an amino acid sequence of Appendix B such that the protein or portion thereof maintains the ability to participate in the metabolism of compounds necessary for the construction of cellular membranes in C. glutamicum, or in the transport of molecules across these membranes. As used herein, the language “sufficiently homologous” refers to proteins or portions thereof which have amino acid sequences which include a minimum number of identical or equivalent (e.g., an amino acid residue which has a similar side chain as an amino acid residue in one of the sequences of Appendix B) amino acid residues to an amino acid sequence of Appendix B such that the protein or portion thereof is able to participate in the metabolism of compounds necessary for the construction of cellular membranes in C. glutamicum, or in the transport of molecules across these membranes. Protein members of such membrane component metabolic pathways or membrane transport systems, as described herein, may play a role in the production and secretion of one or more fine chemicals. Examples of such activities are also described herein. Thus, “the function of an MCT protein” contributes either directly or indirectly to the yield, production, and/or efficiency of production of one or more fine chemicals. Examples of MCT protein activities are set forth in Table 1.

In another embodiment, the protein is at least about 50-60%, preferably at least about 60-70%, and more preferably at least about 70-80%, 80-90%, 90-95%, and most preferably at least about 96%, 97%, 98%, 99% or more homologous to an entire amino acid sequence of Appendix B.

Portions of proteins encoded by the MCT nucleic acid molecules of the invention are preferably biologically active portions of one of the MCT proteins. As used herein, the term “biologically active portion of an MCT protein” is intended to include a portion, e.g., a domain/motif, of an MCT protein that participates in the metabolism of compounds necessary for the construction of cellular membranes in C. glutamicum, or in the transport of molecules across these membranes, or has an activity as set forth in Table 1. To determine whether an MCT protein or a biologically active portion thereof can participate in the metabolism of compounds necessary for the construction of cellular membranes in C. glutamicum, or in the transport of molecules across these membranes, an assay of enzymatic activity may be performed. Such assay methods are well known to those of ordinary skill in the art, as detailed in Example 8 of the Exemplification.

Additional nucleic acid fragments encoding biologically active portions of an MCT protein can be prepared by isolating a portion of one of the sequences in Appendix B, expressing the encoded portion of the MCT protein or peptide (e.g., by recombinant expression in vitro) and assessing the activity of the encoded portion of the MCT protein or peptide.

The invention further encompasses nucleic acid molecules that differ from one of the nucleotide sequences shown in Appendix A (and portions thereof) due to degeneracy of the genetic code and thus encode the same MCT protein as that encoded by the nucleotide sequences shown in Appendix A. In another embodiment, an isolated nucleic acid molecule of the invention has a nucleotide sequence encoding a protein having an amino acid sequence shown in Appendix B. In a still further embodiment, the nucleic acid molecule of the invention encodes a full length C. glutamicum protein which is substantially homologous to an amino acid sequence of Appendix B (encoded by an open reading frame shown in Appendix A).

It will be understood by one of ordinary skill in the art that in one embodiment the sequences of the invention are not meant to include the sequences of the prior art, such as those Genbank sequences set forth in Tables 2 or 4 which were available prior to the present invention. In one embodiment, the invention includes nucleotide and amino acid sequences having a percent identity to a nucleotide or amino acid sequence of the invention which is greater than that of a sequence of the prior art (e.g., a Genbank sequence (or the protein encoded by such a sequence) set forth in Tables 2 or 4). For example, the invention includes a nucleotide sequence which is greater than and/or at least 50% identical to the nucleotide sequence designated RXA00777 (SEQ ID NO:5), a nucleotide sequence which is greater than and/or at least 40% identical to the nucleotide sequence designated RXA02439 (SEQ ID NO:17), and a nucleotide sequence which is greater than and/or at least 39% identical to the nucleotide sequence designated RXA00002 (SEQ ID NO:23). One of ordinary skill in the art would be able to calculate the lower threshold of percent identity for any given sequence of the invention by examining the GAP-calculated percent identity scores set forth in Table 4 for each of the three top hits for the given sequence, and by subtracting the highest GAP-calculated percent identity from 100 percent. One of ordinary skill in the art will also appreciate that nucleic acid and amino acid sequences having percent identities greater than the lower threshold so calculated (e.g., at least 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more identical) are also encompassed by the invention.

In addition to the C. glutamicum MCT nucleotide sequences shown in Appendix A, it will be appreciated by one of ordinary skill in the art that DNA sequence polymorphisms that lead to changes in the amino acid sequences of MCT proteins may exist within a population (e.g., the C. glutamicum population). Such genetic polymorphism in the MCT gene may exist among individuals within a population due to natural variation. As used herein, the terms “gene” and “recombinant gene” refer to nucleic acid molecules comprising an open reading frame encoding an MCT protein, preferably a C. glutamicum MCT protein. Such natural variations can typically result in 1-5% variance in the nucleotide sequence of the MCT gene. Any and all such nucleotide variations and resulting amino acid polymorphisms in MCT that are the result of natural variation and that do not alter the functional activity of MCT proteins are intended to be within the scope of the invention.

Nucleic acid molecules corresponding to natural variants and non-C. glutamicum homologues of the C. glutamicum MCT DNA of the invention can be isolated based on their homology to the C. glutamicum MCT nucleic acid disclosed herein using the C. glutamicum DNA, or a portion thereof, as a hybridization probe according to standard hybridization techniques under stringent hybridization conditions. Accordingly, in another embodiment, an isolated nucleic acid molecule of the invention is at least 15 nucleotides in length and hybridizes under stringent conditions to the nucleic acid molecule comprising a nucleotide sequence of Appendix A. In other embodiments, the nucleic acid is at least 30, 50, 100, 250 or more nucleotides in length. As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 65%, more preferably at least about 70%, and even more preferably at least about 75% or more homologous to each other typically remain hybridized to each other. Such stringent conditions are known to those of ordinary skill in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to a sequence of Appendix A corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein). In one embodiment, the nucleic acid encodes a natural C. glutamicum MCT protein.

In addition to naturally-occurring variants of the MCT sequence that may exist in the population, one of ordinary skill in the art will further appreciate that changes can be introduced by mutation into a nucleotide sequence of Appendix A, thereby leading to changes in the amino acid sequence of the encoded MCT protein, without altering the functional ability of the MCT protein. For example, nucleotide substitutions leading to amino acid substitutions at “non-essential” amino acid residues can be made in a sequence of Appendix A. A “non-essential” amino acid residue is a residue that can be altered from the wild-type sequence of one of the MCT proteins (Appendix B) without altering the activity of said MCT protein, whereas an “essential” amino acid residue is required for MCT protein activity. Other amino acid residues, however, (e.g., those that are not conserved or only semi-conserved in the domain having MCT activity) may not be essential for activity and thus are likely to be amenable to alteration without altering MCT activity.

Accordingly, another aspect of the invention pertains to nucleic acid molecules encoding MCT proteins that contain changes in amino acid residues that are not essential for MCT activity. Such MCT proteins differ in amino acid sequence from a sequence contained in Appendix B yet retain at least one of the MCT activities described herein. In one embodiment, the isolated nucleic acid molecule comprises a nucleotide sequence encoding a protein, wherein the protein comprises an amino acid sequence at least about 50% homologous to an amino acid sequence of Appendix B and is capable of participate in the metabolism of compounds necessary for the construction of cellular membranes in C. glutamicum, or in the transport of molecules across these membranes, or has one or more activities set forth in Table 1. Preferably, the protein encoded by the nucleic acid molecule is at least about 50-60% homologous to one of the sequences in Appendix B, more preferably at least about 60-70% homologous to one of the sequences in Appendix B, even more preferably at least about 70-80%, 80-90%, 90-95% homologous to one of the sequences in Appendix B, and most preferably at least about 96%, 97%, 98%, or 99% homologous to one of the sequences in Appendix B.

To determine the percent homology of two amino acid sequences (e.g., one of the sequences of Appendix B and a mutant form thereof) or of two nucleic acids, the sequences are aligned for optimal comparison purposes (e.g., gaps can be introduced in the sequence of one protein or nucleic acid for optimal alignment with the other protein or nucleic acid). The amino acid residues or nucleotides at corresponding amino acid positions or nucleotide positions are then compared. When a position in one sequence (e.g., one of the sequences of Appendix B) is occupied by the same amino acid residue or nucleotide as the corresponding position in the other sequence (e.g., a mutant form of the sequence selected from Appendix B), then the molecules are homologous at that position (i.e., as used herein amino acid or nucleic acid “homology” is equivalent to amino acid or nucleic acid “identity”). The percent homology between the two sequences is a function of the number of identical positions shared by the sequences (i.e., % homology=# of identical positions/total # of positions×100).

An isolated nucleic acid molecule encoding an MCT protein homologous to a protein sequence of Appendix B can be created by introducing one or more nucleotide substitutions, additions or deletions into a nucleotide sequence of Appendix A such that one or more amino acid substitutions, additions or deletions are introduced into the encoded protein. Mutations can be introduced into one of the sequences of Appendix A by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted nonessential amino acid residue in an MCT protein is preferably replaced with another amino acid residue from the same side chain family. Alternatively, in another embodiment, mutations can be introduced randomly along all or part of an MCT coding sequence, such as by saturation mutagenesis, and the resultant mutants can be screened for an MCT activity described herein to identify mutants that retain MCT activity. Following mutagenesis of one of the sequences of Appendix A, the encoded protein can be expressed recombinantly and the activity of the protein can be determined using, for example, assays described herein (see Example 8 of the Exemplification).

In addition to the nucleic acid molecules encoding MCT proteins described above, another aspect of the invention pertains to isolated nucleic acid molecules which are antisense thereto. An “antisense” nucleic acid comprises a nucleotide sequence which is complementary to a “sense” nucleic acid encoding a protein, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire MCT coding strand, or to only a portion thereof. In one embodiment, an antisense nucleic acid molecule is antisense to a “coding region” of the coding strand of a nucleotide sequence encoding an MCT protein. The term “coding region” refers to the region of the nucleotide sequence comprising codons which are translated into amino acid residues (e.g., the entire coding region of NO. 3 (RXA00777) comprises nucleotides 1 to 1065). In another embodiment, the antisense nucleic acid molecule is antisense to a “noncoding region” of the coding strand of a nucleotide sequence encoding MCT. The term “noncoding region” refers to 5′ and 3′ sequences which flank the coding region that are not translated into amino acids (i.e., also referred to as 5′ and 3′ untranslated regions).

Given the coding strand sequences encoding MCT disclosed herein (e.g., the sequences set forth in Appendix A), antisense nucleic acids of the invention can be designed according to the rules of Watson and Crick base pairing. The antisense nucleic acid molecule can be complementary to the entire coding region of MCT mRNA, but more preferably is an oligonucleotide which is antisense to only a portion of the coding or noncoding region of MCT mRNA. For example, the antisense oligonucleotide can be complementary to the region surrounding the translation start site of MCT mRNA. An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a cell or generated in situ such that they hybridize with or bind to cellular mRNA and/or genomic DNA encoding an MCT protein to thereby inhibit expression of the protein, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. The antisense molecule can be modified such that it specifically binds to a receptor or an antigen expressed on a selected cell surface, e.g., by linking the antisense nucleic acid molecule to a peptide or an antibody which binds to a cell surface receptor or antigen. The antisense nucleic acid molecule can also be delivered to cells using the vectors described herein. To achieve sufficient intracellular concentrations of the antisense molecules, vector constructs in which the antisense nucleic acid molecule is placed under the control of a strong prokaryotic, viral, or eukaryotic promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids. Res. 15: 6625-6641). The antisense nucleic acid molecule can also comprise a 2′-o-methylribonucleotide (Inoue et al. (1987) Nucleic Acids Res. 15: 6131-6148) or a chimeric RNA-DNA analogue (Inoue et al. (1987) FEBS Lett. 215: 327-330).

In still another embodiment, an antisense nucleic acid of the invention is a ribozyme. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334: 585-591)) can be used to catalytically cleave MCT mRNA transcripts to thereby inhibit translation of MCT mRNA. A ribozyme having specificity for an MCT-encoding nucleic acid can be designed based upon the nucleotide sequence of an MCT DNA disclosed herein (i.e., NO. 3 (RXA00777 in Appendix A)). For example, a derivative of a Tetrahymena L-19 IVS RNA can be constructed in which the nucleotide sequence of the active site is complementary to the nucleotide sequence to be cleaved in an MCT-encoding mRNA. See, e.g., Cech et al. U.S. Pat. No. 4,987,071 and Cech et al. U.S. Pat. No. 5,116,742. Alternatively, MCT mRNA can be used to select a catalytic RNA having a specific ribonuclease activity from a pool of RNA molecules. See, e.g., Bartel, D. and Szostak, J. W. (1993) Science 261: 1411-1418.

Alternatively, MCT gene expression can be inhibited by targeting nucleotide sequences complementary to the regulatory region of an MCT nucleotide sequence (e.g., an MCT promoter and/or enhancers) to form triple helical structures that prevent transcription of an MCT gene in target cells. See generally, Helene, C. (1991) Anticancer Drug Des. 6(6): 569-84; Helene, C. et al. (1992) Ann. N.Y. Acad. Sci. 660: 27-36; and Maher, L. J. (1992) Bioassays 14(12): 807-15.

B. Recombinant Expression Vectors and Host Cells

Another aspect of the invention pertains to vectors, preferably expression vectors, containing a nucleic acid encoding an MCT protein (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells. Preferred regulatory sequences are, for example, promoters such as cos-, tac-, trp-, tet-, trp-tet-, lpp-, lac-, lpp-lac-, lacI^{q}-, T7-, T5-, T3-, gal-, trc-, ara-, SP6-, arny, SPO2, λ-P_{R}- or λ P_{L}, which are used preferably in bacteria. Additional regulatory sequences are, for example, promoters from yeasts and fungi, such as ADC1, MFα, AC, P-60, CYC1, GAPDH, TEF, rp28, ADH, promoters from plants such as CaMV/35S, SSU, OCS, lib4, usp, STLS1, B33, nos or ubiquitin- or phaseolin-promoters. It is also possible to use artificial promoters. It will be appreciated by one of ordinary skill in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., MCT proteins, mutant forms of MCT proteins, fusion proteins, etc.).

The recombinant expression vectors of the invention can be designed for expression of MCT proteins in prokaryotic or eukaryotic cells. For example, MCT genes can be expressed in bacterial cells such as C. glutamicum, insect cells (using baculovirus expression vectors), yeast and other fungal cells (see Romanos, M. A. et al. (1992) “Foreign gene expression in yeast: a review”, Yeast 8: 423-488; van den Hondel, C. A. M. J. J. et al. (1991) “Heterologous gene expression in filamentous fungi” in: More Gene Manipulations in Fungi, J. W. Bennet & L. L. Lasure, eds., p. 396-428: Academic Press: San Diego; and van den Hondel, C. A. M. J. J. & Punt, P. J. (1991) “Gene transfer systems and vector development for filamentous fungi, in: Applied Molecular Genetics of Fungi, Peberdy, J. F. et al., eds., p. 1-28, Cambridge University Press: Cambridge), algae and multicellular plant cells (see Schmidt, R. and Willmitzer, L. (1988) High efficiency Agrobacterium tumefaciens—mediated transformation of Arabidopsis thaliana leaf and cotyledon explants” Plant Cell Rep.: 583-586), or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out with vectors containing constitutive or inducible promoters directing the expression of either fusion or non-fusion proteins. Fusion vectors add a number of amino acids to a protein encoded therein, usually to the amino terminus of the recombinant protein but also to the C-terminus or fused within suitable regions in the proteins. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase.

Typical fusion expression vectors include pGEX (Pharmacia Biotech Inc; Smith, D. B. and Johnson, K. S. (1988) Gene 67: 31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. In one embodiment, the coding sequence of the MCT protein is cloned into a pGEX expression vector to create a vector encoding a fusion protein comprising, from the N-terminus to the C-terminus, GST-thrombin cleavage site-X protein. The fusion protein can be purified by affinity chromatography using glutathione-agarose resin. Recombinant MCT protein unfused to GST can be recovered by cleavage of the fusion protein with thrombin.

Examples of suitable inducible non-fusion E. coli expression vectors include pTrc (Amann et al., (1988) Gene 69: 301-315) pLG338, pACYC184, pBR322, pUC18, pUC19, pKC30, pRep4, pHS1, pHS2, pPLc236, pMBL24, pLG200, pUR290, pIN-III113-B1, λgt11, pBdCl, and pET 11d (Studier et al., Gene Expression Technology: Methods in Enzymology 185, Academic Press, San Diego, Calif. (1990) 60-89; and Pouwels et al., eds. (1985) Cloning Vectors. Elsevier: New York IBSN 0 444 904018). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS 174(DE3) from a resident λ prophage harboring a T7 gn1 gene under the transcriptional control of the lacUV 5 promoter. For transformation of other varieties of bacteria, appropriate vectors may be selected. For example, the plasmids pIJ101, pIJ364, pIJ702 and pIJ361 are known to be useful in transforming Streptomyces, while plasmids pUB110, pC194, or pBD214 are suited for transformation of Bacillus species. Several plasmids of use in the transfer of genetic information into Corynebacterium include pHM1519, pBL1, pSA77, or pAJ667 (Pouwels et al., eds. (1985) Cloning Vectors. Elsevier: New York IBSN 0 444 904018).

One strategy to maximize recombinant protein expression is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in the bacterium chosen for expression, such as C. glutamicum (Wada et al. (1992) Nucleic Acids Res. 20: 2111-2118). Such alteration of nucleic acid sequences of the invention can be carried out by standard DNA synthesis techniques.

In another embodiment, the MCT protein expression vector is a yeast expression vector. Examples of vectors for expression in yeast S. cerevisiae include pYepSec1 (Baldari, et al., (1987) Embo J. 6: 229-234), 2μ, pAG-1, Yep6, Yep13, pEMBLYe23, pMFa (Kurjan and Herskowitz, (1982) Cell 30: 933-943), pJRY88 (Schultz et al., (1987) Gene 54: 113-123), and pYES2 (Invitrogen Corporation, San Diego, Calif.). Vectors and methods for the construction of vectors appropriate for use in other fungi, such as the filamentous fungi, include those detailed in: van den Hondel, C. A. M. J. J. & Punt, P. J. (1991) “Gene transfer systems and vector development for filamentous fungi, in: Applied Molecular Genetics of Fungi, J. F. Peberdy, et al., eds., p. 1-28, Cambridge University Press: Cambridge, and Pouwels et al., eds. (1985) Cloning Vectors. Elsevier: New York (IBSN 0 444 904018).

Alternatively, the MCT proteins of the invention can be expressed in insect cells using baculovirus expression vectors. Baculovirus vectors available for expression of proteins in cultured insect cells (e.g., Sf9 cells) include the pAc series (Smith et al. (1983) Mol. Cell Biol. 3: 2156-2165) and the pVL series (Lucklow and Summers (1989) Virology 170: 31-39).

In another embodiment, the MCT proteins of the invention may be expressed in unicellular plant cells (such as algae) or in plant cells from higher plants (e.g., the spermatophytes, such as crop plants). Examples of plant expression vectors include those detailed in: Becker, D., Kemper, E., Schell, J. and Masterson, R. (1992) “New plant binary vectors with selectable markers located proximal to the left border”, Plant Mol. Biol. 20: 1195-1197; and Bevan, M. W. (1984) “Binary Agrobacterium vectors for plant transformation”, Nucl. Acid. Res. 12: 8711-8721, and include pLGV23, pGHlac+, pBIN19, pAK2004, and pDH51 (Pouwels et al., eds. (1985) Cloning Vectors. Elsevier: New York IBSN 0 444 904018).

In yet another embodiment, a nucleic acid of the invention is expressed in mammalian cells using a mammalian expression vector. Examples of mammalian expression vectors include pCDM8 (Seed, B. (1987) Nature 329: 840) and pMT2PC (Kaufman et al. (1987) EMBO J. 6: 187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1: 268-277), lymphoid-specific promoters (Calame and Eaton (1988) Adv. Immunol. 43: 235-275), in particular promoters of T cell receptors (Winoto and Baltimore (1989) EMBO J. 8: 729-733) and immunoglobulins (Banerji et al. (1983) Cell 33: 729-740; Queen and Baltimore (1983) Cell 33: 741-748), neuron-specific promoters (e.g., the neurofilament promoter; Byrne and Ruddle (1989) PNAS 86: 5473-5477), pancreas-specific promoters (Edlund et al. (1985) Science 230: 912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249: 374-379) and the α-fetoprotein promoter (Campes and Tilghman (1989) Genes Dev. 3: 537-546).

The invention further provides a recombinant expression vector comprising a DNA molecule of the invention cloned into the expression vector in an antisense orientation. That is, the DNA molecule is operatively linked to a regulatory sequence in a manner which allows for expression (by transcription of the DNA molecule) of an RNA molecule which is antisense to MCT mRNA. Regulatory sequences operatively linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, an MCT protein can be expressed in bacterial cells such as C. glutamicum, insect cells, yeast or mammalian cells (such as Chinese hamster ovary cells (CHO) or COS cells). Other suitable host cells are known to one of ordinary skill in the art. Microorganisms related to Corynebacterium glutamicum which may be conveniently used as host cells for the nucleic acid and protein molecules of the invention are set forth in Table 3.

Vector DNA can be introduced into prokaryotic or eukaryotic cells via conventional transformation or transfection techniques. As used herein, the terms “transformation” and “transfection”, “conjugation” and “transduction” are intended to refer to a variety of art-recognized techniques for introducing foreign nucleic acid (e.g., linear DNA or RNA (e.g., a linearized vector or a gene construct alone without a vector) or nucleic acid in the form of a vector (e.g., a plasmid, phage, phasmid, phagemid, transposon or other DNA) into a host cell, including calcium phosphate or calcium chloride co-precipitation, DEAE-dextran-mediated transfection, lipofection, natural competence, chemical-mediated transfer, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding an MCT protein or can be introduced on a separate vector. Cells stably transfected with the introduced nucleic acid can be identified by, for example, drug selection (e.g., cells that have incorporated the selectable marker gene will survive, while the other cells die).

To create a homologous recombinant microorganism, a vector is prepared which contains at least a portion of an MCT gene into which a deletion, addition or substitution has been introduced to thereby alter, e.g., functionally disrupt, the MCT gene. Preferably, this MCT gene is a Corynebacterium glutamicum MCT gene, but it can be a homologue from a related bacterium or even from a mammalian, yeast, or insect source. In a preferred embodiment, the vector is designed such that, upon homologous recombination, the endogenous MCT gene is functionally disrupted (i.e., no longer encodes a functional protein; also referred to as a “knock out” vector). Alternatively, the vector can be designed such that, upon homologous recombination, the endogenous MCT gene is mutated or otherwise altered but still encodes functional protein (e.g., the upstream regulatory region can be altered to thereby alter the expression of the endogenous MCT protein). In the homologous recombination vector, the altered portion of the MCT gene is flanked at its 5′ and 3′ ends by additional nucleic acid of the MCT gene to allow for homologous recombination to occur between the exogenous MCT gene carried by the vector and an endogenous MCT gene in a microorganism. The additional flanking MCT nucleic acid is of sufficient length for successful homologous recombination with the endogenous gene. Typically, several kilobases of flanking DNA (both at the 5′ and 3′ ends) are included in the vector (see e.g., Thomas, K. R., and Capecchi, M. R. (1987) Cell 51: 503 for a description of homologous recombination vectors). The vector is introduced into a microorganism (e.g., by electroporation) and cells in which the introduced MCT gene has homologously recombined with the endogenous MCT gene are selected, using art-known techniques.

In another embodiment, recombinant microorganisms can be produced which contain selected systems which allow for regulated expression of the introduced gene. For example, inclusion of an MCT gene on a vector placing it under control of the lac operon permits expression of the MCT gene only in the presence of IPTG. Such regulatory systems are well known in the art.

In another embodiment, an endogenous MCT gene in a host cell is disrupted (e.g., by homologous recombination or other genetic means known in the art) such that expression of its protein product does not occur. In another embodiment, an endogenous or introduced MCT gene in a host cell has been altered by one or more point mutations, deletions, or inversions, but still encodes a functional MCT protein. In still another embodiment, one or more of the regulatory regions (e.g., a promoter, repressor, or inducer) of an MCT gene in a microorganism has been altered (e.g., by deletion, truncation, inversion, or point mutation) such that the expression of the MCT gene is modulated. One of ordinary skill in the art will appreciate that host cells containing more than one of the described MCT gene and protein modifications may be readily produced using the methods of the invention, and are meant to be included in the present invention.

A host cell of the invention, such as a prokaryotic or eukaryotic host cell in culture, can be used to produce (i.e., express) an MCT protein. Accordingly, the invention further provides methods for producing MCT proteins using the host cells of the invention. In one embodiment, the method comprises culturing the host cell of invention (into which a recombinant expression vector encoding an MCT protein has been introduced, or into which genome has been introduced a gene encoding a wild-type or altered MCT protein) in a suitable medium until MCT protein is produced. In another embodiment, the method further comprises isolating MCT proteins from the medium or the host cell.

C. Isolated MCT Proteins

Another aspect of the invention pertains to isolated MCT proteins, and biologically active portions thereof. An “isolated” or “purified” protein or biologically active portion thereof is substantially free of cellular material when produced by recombinant DNA techniques, or chemical precursors or other chemicals when chemically synthesized. The language “substantially free of cellular material” includes preparations of MCT protein in which the protein is separated from cellular components of the cells in which it is naturally or recombinantly produced. In one embodiment, the language “substantially free of cellular material” includes preparations of MCT protein having less than about 30% (by dry weight) of non-MCT protein (also referred to herein as a “contaminating protein”), more preferably less than about 20% of non-MCT protein, still more preferably less than about 10% of non-MCT protein, and most preferably less than about 5% non-MCT protein. When the MCT protein or biologically active portion thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other chemicals” includes preparations of MCT protein in which the protein is separated from chemical precursors or other chemicals which are involved in the synthesis of the protein. In one embodiment, the language “substantially free of chemical precursors or other chemicals” includes preparations of MCT protein having less than about 30% (by dry weight) of chemical precursors or non-MCT chemicals, more preferably less than about 20% chemical precursors or non-MCT chemicals, still more preferably less than about 10% chemical precursors or non-MCT chemicals, and most preferably less than about 5% chemical precursors or non-MCT chemicals. In preferred embodiments, isolated proteins or biologically active portions thereof lack contaminating proteins from the same organism from which the MCT protein is derived. Typically, such proteins are produced by recombinant expression of, for example, a C. glutamicum MCT protein in a microorganism such as C. glutamicum.

An isolated MCT protein or a portion thereof of the invention can participate in the metabolism of compounds necessary for the construction of cellular membranes in C. glutamicum, or in the transport of molecules across these membranes, or has one or more of the activities set forth in Table 1. In preferred embodiments, the protein or portion thereof comprises an amino acid sequence which is sufficiently homologous to an amino acid sequence of Appendix B such that the protein or portion thereof maintains the ability participate in the metabolism of compounds necessary for the construction of cellular membranes in C. glutamicum, or in the transport of molecules across these membranes. The portion of the protein is preferably a biologically active portion as described herein. In another preferred embodiment, an MCT protein of the invention has an amino acid sequence shown in Appendix B. In yet another preferred embodiment, the MCT protein has an amino acid sequence which is encoded by a nucleotide sequence which hybridizes, e.g., hybridizes under stringent conditions, to a nucleotide sequence of Appendix A. In still another preferred embodiment, the MCT protein has an amino acid sequence which is encoded by a nucleotide sequence that is at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more homologous to one of the nucleic acid sequences of Appendix A, or a portion thereof. Ranges and identity values intermediate to the above-recited values, (e.g., 70-90% identical or 80-95% identical) are also intended to be encompassed by the present invention. For example, ranges of identity values using a combination of any of the above values recited as upper and/or lower limits are intended to be included. The preferred MCT proteins of the present invention also preferably possess at least one of the MCT activities described herein. For example, a preferred MCT protein of the present invention includes an amino acid sequence encoded by a nucleotide sequence which hybridizes, e.g., hybridizes under stringent conditions, to a nucleotide sequence of Appendix A, and which can participate in the metabolism of compounds necessary for the construction of cellular membranes in C. glutamicum, or in the transport of molecules across these membranes, or which has one or more of the activities set forth in Table 1.

In other embodiments, the MCT protein is substantially homologous to an amino acid sequence of Appendix B and retains the functional activity of the protein of one of the sequences of Appendix B yet differs in amino acid sequence due to natural variation or mutagenesis, as described in detail in subsection I above. Accordingly, in another embodiment, the MCT protein is a protein which comprises an amino acid sequence which is at least about 50%, 51%, 52%, 53%, 54%, 55%, 56%, 57%, 58%, 59%, or 60%, preferably at least about 61%, 62%, 63%, 64%, 65%, 66%, 67%, 68%, 69%, or 70%, more preferably at least about 71%, 72%, 73%, 74%, 75%, 76%, 77%, 78%, 79%, or 80%, 81%, 82%, 83%, 84%, 85%, 86%, 87%, 88%, 89%, or 90%, or 91%, 92%, 93%, 94%, and even more preferably at least about 95%, 96%, 97%, 98%, 99% or more homologous to an entire amino acid sequence of Appendix B and which has at least one of the MCT activities described herein. Ranges and identity values intermediate to the above-recited values, (e.g., 70-90% identical or 80-95% identical) are also intended to be encompassed by the present invention. For example, ranges of identity values using a combination of any of the above values recited as upper and/or lower limits are intended to be included. In another embodiment, the invention pertains to a full length C. glutamicum protein which is substantially homologous to an entire amino acid sequence of Appendix B.

Biologically active portions of an MCT protein include peptides comprising amino acid sequences derived from the amino acid sequence of an MCT protein, e.g., the an amino acid sequence shown in Appendix B or the amino acid sequence of a protein homologous to an MCT protein, which include fewer amino acids than a full length MCT protein or the full length protein which is homologous to an MCT protein, and exhibit at least one activity of an MCT protein. Typically, biologically active portions (peptides, e.g., peptides which are, for example, 5, 10, 15, 20, 30, 35, 36, 37, 38, 39, 40, 50, 100 or more amino acids in length) comprise a domain or motif with at least one activity of an MCT protein. Moreover, other biologically active portions, in which other regions of the protein are deleted, can be prepared by recombinant techniques and evaluated for one or more of the activities described herein. Preferably, the biologically active portions of an MCT protein include one or more selected domains/motifs or portions thereof having biological activity.

MCT proteins are preferably produced by recombinant DNA techniques. For example, a nucleic acid molecule encoding the protein is cloned into an expression vector (as described above), the expression vector is introduced into a host cell (as described above) and the MCT protein is expressed in the host cell. The MCT protein can then be isolated from the cells by an appropriate purification scheme using standard protein purification techniques. Alternative to recombinant expression, an MCT protein, polypeptide, or peptide can be synthesized chemically using standard peptide synthesis techniques. Moreover, native MCT protein can be isolated from cells (e.g., endothelial cells), for example using an anti-MCT antibody, which can be produced by standard techniques utilizing an MCT protein or fragment thereof of this invention.

The invention also provides MCT chimeric or fusion proteins. As used herein, an MCT “chimeric protein” or “fusion protein” comprises an MCT polypeptide operatively linked to a non-MCT polypeptide. An “MCT polypeptide” refers to a polypeptide having an amino acid sequence corresponding to an MCT protein, whereas a “non-MCT polypeptide” refers to a polypeptide having an amino acid sequence corresponding to a protein which is not substantially homologous to the MCT protein, e.g., a protein which is different from the MCT protein and which is derived from the same or a different organism. Within the fusion protein, the term “operatively linked” is intended to indicate that the MCT polypeptide and the non-MCT polypeptide are fused in-frame to each other. The non-MCT polypeptide can be fused to the N-terminus or C-terminus of the MCT polypeptide. For example, in one embodiment the fusion protein is a GST-MCT fusion protein in which the MCT sequences are fused to the C-terminus of the GST sequences. Such fusion proteins can facilitate the purification of recombinant MCT proteins. In another embodiment, the fusion protein is an MCT protein containing a heterologous signal sequence at its N-terminus. In certain host cells (e.g., mammalian host cells), expression and/or secretion of an MCT protein can be increased through use of a heterologous signal sequence.

Preferably, an MCT chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). An MCT-encoding nucleic acid can be cloned into such an expression vector such that the fusion moiety is linked in-frame to the MCT protein.

Homologues of the MCT protein can be generated by mutagenesis, e.g., discrete point mutation or truncation of the MCT protein. As used herein, the term “homologue” refers to a variant form of the MCT protein which acts as an agonist or antagonist of the activity of the MCT protein. An agonist of the MCT protein can retain substantially the same, or a subset, of the biological activities of the MCT protein. An antagonist of the MCT protein can inhibit one or more of the activities of the naturally occurring form of the MCT protein, by, for example, competitively binding to a downstream or upstream member of the cell membrane component metabolic cascade which includes the MCT protein, or by binding to an MCT protein which mediates transport of compounds across such membranes, thereby preventing translocation from taking place.

In an alternative embodiment, homologues of the MCT protein can be identified by screening combinatorial libraries of mutants, e.g., truncation mutants, of the MCT protein for MCT protein agonist or antagonist activity. In one embodiment, a variegated library of MCT variants is generated by combinatorial mutagenesis at the nucleic acid level and is encoded by a variegated gene library. A variegated library of MCT variants can be produced by, for example, enzymatically ligating a mixture of synthetic oligonucleotides into gene sequences such that a degenerate set of potential MCT sequences is expressible as individual polypeptides, or alternatively, as a set of larger fusion proteins (e.g., for phage display) containing the set of MCT sequences therein. There are a variety of methods which can be used to produce libraries of potential MCT homologues from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential MCT sequences. Methods for synthesizing degenerate oligonucleotides are known in the art (see, e.g., Narang, S. A. (1983) Tetrahedron 39: 3; Itakura et al. (1984) Annu. Rev. Biochem. 53: 323; Itakura et al. (1984) Science 198: 1056; Ike et al. (1983) Nucleic Acid Res. 11: 477.

In addition, libraries of fragments of the MCT protein coding can be used to generate a variegated population of MCT fragments for screening and subsequent selection of homologues of an MCT protein. In one embodiment, a library of coding sequence fragments can be generated by treating a double stranded PCR fragment of an MCT coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the MCT protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of MCT homologues. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify MCT homologues (Arkin and Yourvan (1992) PNAS 89: 7811-7815; Delgrave et al. (1993) Protein Engineering 6(3): 327-331).

In another embodiment, cell based assays can be exploited to analyze a variegated MCT library, using methods well known in the art.

D. Uses and Methods of the Invention

The nucleic acid molecules, proteins, protein homologues, fusion proteins, primers, vectors, and host cells described herein can be used in one or more of the following methods: identification of C. glutamicum and related organisms; mapping of genomes of organisms related to C. glutamicum; identification and localization of C. glutamicum sequences of interest; evolutionary studies; determination of MCT protein regions required for function; modulation of an MCT protein activity; modulation of the metabolism of one or more cell membrane components; modulation of the transmembrane transport of one or more compounds; and modulation of cellular production of a desired compound, such as a fine chemical.

The MCT nucleic acid molecules of the invention have a variety of uses. First, they may be used to identify an organism as being Corynebacterium glutamicum or a close relative thereof. Also, they may be used to identify the presence of C. glutamicum or a relative thereof in a mixed population of microorganisms. The invention provides the nucleic acid sequences of a number of C. glutamicum genes; by probing the extracted genomic DNA of a culture of a unique or mixed population of microorganisms under stringent conditions with a probe spanning a region of a C. glutamicum gene which is unique to this organism, one can ascertain whether this organism is present.

Although Corynebacterium glutamicum itself is nonpathogenic, it is related to pathogenic species, such as Corynebacterium diphtheriae. Corynebacterium diphtheriae is the causative agent of diphtheria, a rapidly developing, acute, febrile infection which involves both local and systemic pathology. In this disease, a local lesion develops in the upper respiratory tract and involves necrotic injury to epithelial cells; the bacilli secrete toxin which is disseminated through this lesion to distal susceptible tissues of the body. Degenerative changes brought about by the inhibition of protein synthesis in these tissues, which include heart, muscle, peripheral nerves, adrenals, kidneys, liver and spleen, result in the systemic pathology of the disease. Diphtheria continues to have high incidence in many parts of the world, including Africa, Asia, Eastern Europe and the independent states of the former Soviet Union. An ongoing epidemic of diphtheria in the latter two regions has resulted in at least 5,000 deaths since 1990. In one embodiment, the invention provides a method of identifying the presence or activity of Cornyebacterium diphtheriae in a subject. This method includes detection of one or more of the nucleic acid or amino acid sequences of the invention (e.g., the sequences set forth in Appendix A or Appendix B) in a subject, thereby detecting the presence or activity of Corynebacterium diphtheriae in the subject. C. glutamicum and C. diphtheriae are related bacteria, and many of the nucleic acid and protein molecules in C. glutamicum are homologous to C. diphtheriae nucleic acid and protein molecules, and can therefore be used to detect C. diphtheriae in a subject.

The nucleic acid and protein molecules of the invention may also serve as markers for specific regions of the genome. This has utility not only in the mapping of the genome, but also for functional studies of C. glutamicum proteins. For example, to identify the region of the genome to which a particular C. glutamicum DNA-binding protein binds, the C. glutamicum genome could be digested, and the fragments incubated with the DNA-binding protein. Those which bind the protein may be additionally probed with the nucleic acid molecules of the invention, preferably with readily detectable labels; binding of such a nucleic acid molecule to the genome fragment enables the localization of the fragment to the genome map of C. glutamicum, and, when performed multiple times with different enzymes, facilitates a rapid determination of the nucleic acid sequence to which the protein binds. Further, the nucleic acid molecules of the invention may be sufficiently homologous to the sequences of related species such that these nucleic acid molecules may serve as markers for the construction of a genomic map in related bacteria, such as Brevibacterium lactofermentum.

The MCT nucleic acid molecules of the invention are also useful for evolutionary and protein structural studies. The metabolic and transport processes in which the molecules of the invention participate are utilized by a wide variety of prokaryotic and eukaryotic cells; by comparing the sequences of the nucleic acid molecules of the present invention to those encoding similar enzymes from other organisms, the evolutionary relatedness of the organisms can be assessed. Similarly, such a comparison permits an assessment of which regions of the sequence are conserved and which are not, which may aid in determining those regions of the protein which are essential for the functioning of the enzyme. This type of determination is of value for protein engineering studies and may give an indication of what the protein can tolerate in terms of mutagenesis without losing function.

Manipulation of the MCT nucleic acid molecules of the invention may result in the production of MCT proteins having functional differences from the wild-type MCT proteins. These proteins may be improved in efficiency or activity, may be present in greater numbers in the cell than is usual, or may be decreased in efficiency or activity.

The invention provides methods for screening molecules which modulate the activity of an MCT protein, either by interacting with the protein itself or a substrate or binding partner of the MCT protein, or by modulating the transcription or translation of an MCT nucleic acid molecule of the invention. In such methods, a microorganism expressing one or more MCT proteins of the invention is contacted with one or more test compounds, and the effect of each test compound on the activity or level of expression of the MCT protein is assessed.

There are a number of mechanisms by which the alteration of an MCT protein of the invention may directly affect the yield, production, and/or efficiency of production of a fine chemical from a C. glutamicum strain incorporating such an altered protein. Recovery of fine chemical compounds from large-scale cultures of C. glutamicum is significantly improved if C. glutamicum secretes the desired compounds, since such compounds may be readily purified from the culture medium (as opposed to extracted from the mass of C. glutamicum cells). By either increasing the number or the activity of transporter molecules which export fine chemicals from the cell, it may be possible to increase the amount of the produced fine chemical which is present in the extracellular medium, thus permitting greater ease of harvesting and purification. Conversely, in order to efficiently overproduce one or more fine chemicals, increased amounts of the cofactors, precursor molecules, and intermediate compounds for the appropriate biosynthetic pathways are required. Therefore, by increasing the number and/or activity of transporter proteins involved in the import of nutrients, such as carbon sources (i.e., sugars), nitrogen sources (i.e., amino acids, ammonium salts), phosphate, and sulfur, it may be possible to improve the production of a fine chemical, due to the removal of any nutrient supply limitations on the biosynthetic process. Further, fatty acids and lipids are themselves desirable fine chemicals, so by optimizing the activity or increasing the number of one or more MCT proteins of the invention which participate in the biosynthesis of these compounds, or by impairing the activity of one or more MCT proteins which are involved in the degradation of these compounds, it may be possible to increase the yield, production, and/or efficiency of production of fatty acid and lipid molecules from C. glutamicum.

The engineering of one or more MCT genes of the invention may also result in MCT proteins having altered activities which indirectly impact the production of one or more desired fine chemicals from C. glutamicum. For example, the normal biochemical processes of metabolism result in the production of a variety of waste products (e.g., hydrogen peroxide and other reactive oxygen species) which may actively interfere with these same metabolic processes (for example, peroxynitrite is known to nitrate tyrosine side chains, thereby inactivating some enzymes having tyrosine in the active site (Groves, J. T. (1999) Curr. Opin. Chem. Biol. 3(2): 226-235). While these waste products are typically excreted, the C. glutamicum strains utilized for large-scale fermentative production are optimized for the overproduction of one or more fine chemicals, and thus may produce more waste products than is typical for a wild-type C. glutamicum. By optimizing the activity of one or more MCT proteins of the invention which are involved in the export of waste molecules, it may be possible to improve the viability of the cell and to maintain efficient metabolic activity. Also, the presence of high intracellular levels of the desired fine chemical may actually be toxic to the cell, so by increasing the ability of the cell to secrete these compounds, one may improve the viability of the cell.

Further, the MCT proteins of the invention may be manipulated such that the relative amounts of various lipid and fatty acid molecules produced are altered. This may have a profound effect on the lipid composition of the membrane of the cell. Since each type of lipid has different physical properties, an alteration in the lipid composition of a membrane may significantly alter membrane fluidity. Changes in membrane fluidity can impact the transport of molecules across the membrane, which, as previously explicated, may modify the export of waste products or the produced fine chemical or the import of necessary nutrients. Such membrane fluidity changes may also profoundly affect the integrity of the cell; cells with relatively weaker membranes are more vulnerable in the large-scale fermentor environment to mechanical stresses which may damage or kill the cell. By manipulating MCT proteins involved in the production of fatty acids and lipids for membrane construction such that the resulting membrane has a membrane composition more amenable to the environmental conditions extant in the cultures utilized to produce fine chemicals, a greater proportion of the C. glutamicum cells should survive and multiply. Greater numbers of C. glutamicum cells in a culture should translate into greater yields, production, or efficiency of production of the fine chemical from the culture.

The aforementioned mutagenesis strategies for MCT proteins to result in increased yields of a fine chemical from C. glutamicum are not meant to be limiting; variations on these strategies will be readily apparent to one of ordinary skill in the art. Using such strategies, and incorporating the mechanisms disclosed herein, the nucleic acid and protein molecules of the invention may be utilized to generate C. glutamicum or related strains of bacteria expressing mutated MCT nucleic acid and protein molecules such that the yield, production, and/or efficiency of production of a desired compound is improved. This desired compound may be any natural product of C. glutamicum, which includes the final products of biosynthesis pathways and intermediates of naturally-occurring metabolic pathways, as well as molecules which do not naturally occur in the metabolism of C. glutamicum, but which are produced by a C. glutamicum strain of the invention.

This invention is further illustrated by the following examples which should not be construed as limiting. The contents of all references, patent applications, patents, published patent applications, Tables, Appendices, and the sequence listing cited throughout this application are hereby incorporated by reference.

### EXEMPLIFICATION

### Example 1

### Preparation of Total Genomic DNA of Corynebacterium glutamicum ATCC 13032

A culture of Corynebacterium glutamicum (ATCC 13032) was grown overnight at 30° C. with vigorous shaking in BHI medium (Difco). The cells were harvested by centrifugation, the supernatant was discarded and the cells were resuspended in 5 ml buffer-I (5% of the original volume of the culture—all indicated volumes have been calculated for 100 ml of culture volume). Composition of buffer-I: 140.34 g/l sucrose, 2.46 g/l MgSO₄×7H₂O, 10 ml/l KH₂PO₄ solution (100 g/l, adjusted to pH 6.7 with KOH), 50 ml/l M12 concentrate (10 g/l (NH₄)₂SO₄, 1 g/l NaCl, 2 g/l MgSO₄×7H₂O, 0.2 g/l CaCl₂, 0.5 g/l yeast extract (Difco), 10 ml/l trace-elements-mix (200 mg/l FeSO₄×H₂O, 10 mg/l ZnSO₄×7H₂O, 3 mg/l MnCl₂×4H₂O, 30 mg/l H₃BO₃ 20 mg/l CoCl₂×6H₂O, 1 mg/l NiCl₂×6H₂O, 3 mg/l Na₂MoO₄×2H₂O, 500 mg/l complexing agent (EDTA or critic acid), 100 ml/l vitamins-mix (0.2 mg/l biotin, 0.2 mg/l folic acid, 20 mg/l p-amino benzoic acid, 20 mg/l riboflavin, 40 mg/l ca-panthothenate, 140 mg/l nicotinic acid, 40 mg/l pyridoxole hydrochloride, 200 mg/l myo-inositol). Lysozyme was added to the suspension to a final concentration of 2.5 mg/ml. After an approximately 4 h incubation at 37° C., the cell wall was degraded and the resulting protoplasts are harvested by centrifugation. The pellet was washed once with 5 ml buffer-I and once with 5 ml TE-buffer (10 mM Tris-HCl, 1 mM EDTA, pH 8). The pellet was resuspended in 4 ml TE-buffer and 0.5 ml SDS solution (10%) and 0.5 ml NaCl solution (5 M) are added. After adding of proteinase K to a final concentration of 200 μg/ml, the suspension is incubated for ca. 18 h at 37° C. The DNA was purified by extraction with phenol, phenol-chloroform-isoamylalcohol and chloroform-isoamylalcohol using standard procedures. Then, the DNA was precipitated by adding 1/50 volume of 3 M sodium acetate and 2 volumes of ethanol, followed by a 30 min incubation at −20° C. and a 30 min centrifugation at 12,000 rpm in a high speed centrifuge using a SS34 rotor (Sorvall). The DNA was dissolved in 1 ml TE-buffer containing 20 μg/ml RNaseA and dialysed at 4° C. against 1000 ml TE-buffer for at least 3 hours. During this time, the buffer was exchanged 3 times. To aliquots of 0.4 ml of the dialysed DNA solution, 0.4 ml of 2 M LiCl and 0.8 ml of ethanol are added. After a 30 min incubation at −20° C., the DNA was collected by centrifugation (13,000 rpm, Biofuge Fresco, Heraeus, Hanau, Germany). The DNA pellet was dissolved in TE-buffer. DNA prepared by this procedure could be used for all purposes, including southern blotting or construction of genomic libraries.

### Example 2

### Construction of Genomic Libraries in Escherichia coli of Corynebacterium glutamicum ATCC13032

Using DNA prepared as described in Example 1, cosmid and plasmid libraries were constructed according to known and well established methods (see e.g., Sambrook, J. et al. (1989) “Molecular Cloning: A Laboratory Manual”, Cold Spring Harbor Laboratory Press, or Ausubel, F. M. et al. (1994) “Current Protocols in Molecular Biology”, John Wiley & Sons.)

Any plasmid or cosmid could be used. Of particular use were the plasmids pBR322 (Sutcliffe, J. G. (1979) Proc. Natl. Acad. Sci. USA, 75: 3737-3741); pACYC177 (Change & Cohen (1978) J. Bacteriol 134: 1141-1156), plasmids of the pBS series (pBSSK+, pBSSK− and others; Stratagene, LaJolla, USA), or cosmids as SuperCos1 (Stratagene, LaJolla, USA) or Lorist6 (Gibson, T. J., Rosenthal A. and Waterson, R. H. (1987) Gene 53: 283-286. Gene libraries specifically for use in C. glutamicum may be constructed using plasmid pSL109 (Lee, H.-S. and A. J. Sinskey (1994) J. Microbiol. Biotechnol. 4: 256-263).

### Example 3

### DNA Sequencing and Computational Functional Analysis

Genomic libraries as described in Example 2 were used for DNA sequencing according to standard methods, in particular by the chain termination method using ABI377 sequencing machines (see e.g., Fleischman, R. D. et al. (1995) “Whole-genome Random Sequencing and Assembly of Haemophilus Influenzae Rd., Science, 269: 496-512). Sequencing primers with the following nucleotide sequences were used: 5′-GGAAACAGTATGACCATG-3′ or 5′-GTAAAACGACGGCCAGT-3′.

### Example 4

### In Vivo Mutagenesis

In vivo mutagenesis of Corynebacterium glutamicum can be performed by passage of plasmid (or other vector) DNA through E. coli or other microorganisms (e.g. Bacillus spp. or yeasts such as Saccharomyces cerevisiae) which are impaired in their capabilities to maintain the integrity of their genetic information. Typical mutator strains have mutations in the genes for the DNA repair system (e.g., mutHLS, mutD, mutT, etc.; for reference, see Rupp, W. D. (1996) DNA repair mechanisms, in: Escherichia coli and Salmonella, p. 2277-2294, ASM: Washington.) Such strains are well known to those of ordinary skill in the art. The use of such strains is illustrated, for example, in Greener, A. and Callahan, M. (1994) Strategies 7: 32-34.

### Example 5

### DNA Transfer Between Escherichia coli and Corynebacterium glutamicum

Several Corynebacterium and Brevibacterium species contain endogenous plasmids (as e.g., pHM1519 or pBL1) which replicate autonomously (for review see, e.g., Martin, J. F. et al. (1987) Biotechnology, 5: 137-146). Shuttle vectors for Escherichia coli and Corynebacterium glutamicum can be readily constructed by using standard vectors for E. coli (Sambrook, J. et al. (1989), “Molecular Cloning: A Laboratory Manual”, Cold Spring Harbor Laboratory Press or Ausubel, F. M. et al. (1994) “Current Protocols in Molecular Biology”, John Wiley & Sons) to which a origin or replication for and a suitable marker from Corynebacterium glutamicum is added. Such origins of replication are preferably taken from endogenous plasmids isolated from Corynebacterium and Brevibacterium species. Of particular use as transformation markers for these species are genes for kanamycin resistance (such as those derived from the Tn5 or Tn903 transposons) or chloramphenicol (Winnacker, E. L. (1987) “From Genes to Clones—Introduction to Gene Technology, VCH, Weinheim). There are numerous examples in the literature of the construction of a wide variety of shuttle vectors which replicate in both E. coli and C. glutamicum, and which can be used for several purposes, including gene over-expression (for reference, see e.g., Yoshihama, M. et al. (1985) J. Bacteriol. 162: 591-597, Martin J. F. et al. (1987) Biotechnology, 5: 137-146 and Eikmanns, B. J. et al. (1991) Gene, 102: 93-98).

Using standard methods, it is possible to clone a gene of interest into one of the shuttle vectors described above and to introduce such a hybrid vectors into strains of Corynebacterium glutamicum. Transformation of C. glutamicum can be achieved by protoplast transformation (Kastsumata, R. et al. (1984) J. Bacteriol. 159306-311), electroporation (Liebl, E. et al. (1989) FEMS Microbiol. Letters, 53: 399-303) and in cases where special vectors are used, also by conjugation (as described e.g. in Schäfer, A et al. (1990) J. Bacteriol. 172: 1663-1666). It is also possible to transfer the shuttle vectors for C. glutamicum to E. coli by preparing plasmid DNA from C. glutamicum (using standard methods well-known in the art) and transforming it into E. coli. This transformation step can be performed using standard methods, but it is advantageous to use an Mcr-deficient E. coli strain, such as NM522 (Gough & Murray (1983) J. Mol. Biol. 166: 1-19).

Genes may be overexpressed in C. glutamicum strains using plasmids which comprise pCG1 (U.S. Pat. No. 4,617,267) or fragments thereof, and optionally the gene for kanamycin resistance from TN903 (Grindley, N. D. and Joyce, C. M. (1980) Proc. Natl. Acad. Sci. USA 77(12): 7176-7180). In addition, genes may be overexpressed in C. glutamicum strains using plasmid pSL109 (Lee, H.-S. and A. J. Sinskey (1994) J. Microbiol. Biotechnol. 4: 256-263).

Aside from the use of replicative plasmids, gene overexpression can also be achieved by integration into the genome. Genomic integration in C. glutamicum or other Corynebacterium or Brevibacterium species may be accomplished by well-known methods, such as homologous recombination with genomic region(s), restriction endonuclease mediated integration (REMI) (see, e.g., DE Patent 19823834), or through the use of transposons. It is also possible to modulate the activity of a gene of interest by modifying the regulatory regions (e.g., a promoter, a repressor, and/or an enhancer) by sequence modification, insertion, or deletion using site-directed methods (such as homologous recombination) or methods based on random events (such as transposon mutagenesis or REMI). Nucleic acid sequences which function as transcriptional terminators may also be inserted 3′ to the coding region of one or more genes of the invention; such terminators are well-known in the art and are described, for example, in Winnacker, E. L. (1987) From Genes to Clones—Introduction to Gene Technology. VCH: Weinheim.

### Example 6

### Assessment of the Expression of the Mutant Protein

Observations of the activity of a mutated protein in a transformed host cell rely on the fact that the mutant protein is expressed in a similar fashion and in a similar quantity to that of the wild-type protein. A useful method to ascertain the level of transcription of the mutant gene (an indicator of the amount of mRNA available for translation to the gene product) is to perform a Northern blot (for reference see, for example, Ausubel et al. (1988) Current Protocols in Molecular Biology, Wiley: New York), in which a primer designed to bind to the gene of interest is labeled with a detectable tag (usually radioactive or chemiluminescent), such that when the total RNA of a culture of the organism is extracted, run on gel, transferred to a stable matrix and incubated with this probe, the binding and quantity of binding of the probe indicates the presence and also the quantity of mRNA for this gene. This information is evidence of the degree of transcription of the mutant gene. Total cellular RNA can be prepared from Corynebacterium glutamicum by several methods, all well-known in the art, such as that described in Bormann, E. R. et al. (1992) Mol. Microbiol. 6: 317-326.

To assess the presence or relative quantity of protein translated from this mRNA, standard techniques, such as a Western blot, may be employed (see, for example, Ausubel et al. (1988) Current Protocols in Molecular Biology, Wiley: New York). In this process, total cellular proteins are extracted, separated by gel electrophoresis, transferred to a matrix such as nitrocellulose, and incubated with a probe, such as an antibody, which specifically binds to the desired protein. This probe is generally tagged with a chemiluminescent or colorimetric label which may be readily detected. The presence and quantity of label observed indicates the presence and quantity of the desired mutant protein present in the cell.

### Example 7

### Growth of Genetically Modified Corynebacterium glutamicum—Media and Culture Conditions

Genetically modified Corynebacteria are cultured in synthetic or natural growth media. A number of different growth media for Corynebacteria are both well-known and readily available (Lieb et al. (1989) Appl. Microbiol. Biotechnol., 32: 205-210; von der Osten et al. (1998) Biotechnology Letters, 11: 11-16; Patent DE 4,120,867; Liebl (1992) “The Genus Corynebacterium, in: The Procaryotes, Volume II, Balows, A. et al., eds. Springer-Verlag). These media consist of one or more carbon sources, nitrogen sources, inorganic salts, vitamins and trace elements. Preferred carbon sources are sugars, such as mono-, di-, or polysaccharides. For example, glucose, fructose, mannose, galactose, ribose, sorbose, ribulose, lactose, maltose, sucrose, raffinose, starch or cellulose serve as very good carbon sources. It is also possible to supply sugar to the media via complex compounds such as molasses or other by-products from sugar refinement. It can also be advantageous to supply mixtures of different carbon sources. Other possible carbon sources are alcohols and organic acids, such as methanol, ethanol, acetic acid or lactic acid. Nitrogen sources are usually organic or inorganic nitrogen compounds, or materials which contain these compounds. Exemplary nitrogen sources include ammonia gas or ammonia salts, such as NH₄Cl or (NH₄)₂SO₄, NH₄OH, nitrates, urea, amino acids or complex nitrogen sources like corn steep liquor, soy bean flour, soy bean protein, yeast extract, meat extract and others.

Inorganic salt compounds which may be included in the media include the chloride-, phosphorous- or sulfate-salts of calcium, magnesium, sodium, cobalt, molybdenum, potassium, manganese, zinc, copper and iron. Chelating compounds can be added to the medium to keep the metal ions in solution. Particularly useful chelating compounds include dihydroxyphenols, like catechol or protocatechuate, or organic acids, such as citric acid. It is typical for the media to also contain other growth factors, such as vitamins or growth promoters, examples of which include biotin, riboflavin, thiamin, folic acid, nicotinic acid, pantothenate and pyridoxin. Growth factors and salts frequently originate from complex media components such as yeast extract, molasses, corn steep liquor and others. The exact composition of the media compounds depends strongly on the immediate experiment and is individually decided for each specific case. Information about media optimization is available in the textbook “Applied Microbiol. Physiology, A Practical Approach (eds. P. M. Rhodes, P. F. Stanbury, IRL Press (1997) pp. 53-73, ISBN 0 19 963577 3). It is also possible to select growth media from commercial suppliers, like standard 1 (Merck) or BHI (grain heart infusion, DIFCO) or others.

All medium components are sterilized, either by heat (20 minutes at 1.5 bar and 121° C.) or by sterile filtration. The components can either be sterilized together or, if necessary, separately. All media components can be present at the beginning of growth, or they can optionally be added continuously or batchwise.

Culture conditions are defined separately for each experiment. The temperature should be in a range between 15° C. and 45° C. The temperature can be kept constant or can be altered during the experiment. The pH of the medium should be in the range of 5 to 8.5, preferably around 7.0, and can be maintained by the addition of buffers to the media. An exemplary buffer for this purpose is a potassium phosphate buffer. Synthetic buffers such as MOPS, HEPES, ACES and others can alternatively or simultaneously be used. It is also possible to maintain a constant culture pH through the addition of NaOH or NH₄OH during growth. If complex medium components such as yeast extract are utilized, the necessity for additional buffers may be reduced, due to the fact that many complex compounds have high buffer capacities. If a fermentor is utilized for culturing the micro-organisms, the pH can also be controlled using gaseous ammonia.

The incubation time is usually in a range from several hours to several days. This time is selected in order to permit the maximal amount of product to accumulate in the broth. The disclosed growth experiments can be carried out in a variety of vessels, such as microtiter plates, glass tubes, glass flasks or glass or metal fermentors of different sizes. For screening a large number of clones, the microorganisms should be cultured in microtiter plates, glass tubes or shake flasks, either with or without baffles. Preferably 100 ml shake flasks are used, filled with 10% (by volume) of the required growth medium. The flasks should be shaken on a rotary shaker (amplitude 25 mm) using a speed-range of 100-300 rpm. Evaporation losses can be diminished by the maintenance of a humid atmosphere; alternatively, a mathematical correction for evaporation losses should be performed.

If genetically modified clones are tested, an unmodified control clone or a control clone containing the basic plasmid without any insert should also be tested. The medium is inoculated to an OD₆₀₀ of 0.5-1.5 using cells grown on agar plates, such as CM plates (10 g/l glucose, 2.5 g/l NaCl, 2 g/l urea, 10 g/l polypeptone, 5 g/l yeast extract, 5 g/l meat extract, 22 g/l NaCl, 2 g/l urea, 10 g/l polypeptone, 5 g/l yeast extract, 5 g/l meat extract, 22 g/l agar, pH 6.8 with 2M NaOH) that had been incubated at 30° C. Inoculation of the media is accomplished by either introduction of a saline suspension of C. glutamicum cells from CM plates or addition of a liquid preculture of this bacterium.

### Example 8

### In Vitro Analysis of the Function of Mutant Proteins

The determination of activities and kinetic parameters of enzymes is well established in the art. Experiments to determine the activity of any given altered enzyme must be tailored to the specific activity of the wild-type enzyme, which is well within the ability of one of ordinary skill in the art. Overviews about enzymes in general, as well as specific details concerning structure, kinetics, principles, methods, applications and examples for the determination of many enzyme activities may be found, for example, in the following references: Dixon, M., and Webb, E. C., (1979) Enzymes. Longmans: London; Fersht, (1985) Enzyme Structure and Mechanism. Freeman: New York; Walsh, (1979) Enzymatic Reaction Mechanisms. Freeman: San Francisco; Price, N.C., Stevens, L. (1982) Fundamentals of Enzymology. Oxford Univ. Press: Oxford; Boyer, P. D., ed. (1983) The Enzymes, 3^{rd} ed. Academic Press: New York; Bisswanger, H., (1994) Enzymkinetik, 2^{nd} ed. VCH: Weinheim (ISBN 3527300325); Bergmeyer, H. U., Bergmeyer, J., Graβl, M., eds. (1983-1986) Methods of Enzymatic Analysis, 3^{rd} ed., vol. I-XII, Verlag Chemie: Weinheim; and Ullmann's Encyclopedia of Industrial Chemistry (1987) vol. A9, “Enzymes”. VCH: Weinheim, p. 352-363.

The activity of proteins which bind to DNA can be measured by several well-established methods, such as DNA band-shift assays (also called gel retardation assays). The effect of such proteins on the expression of other molecules can be measured using reporter gene assays (such as that described in Kolmar, H. et al. (1995) EMBO J. 14: 3895-3904 and references cited therein). Reporter gene test systems are well known and established for applications in both pro- and eukaryotic cells, using enzymes such as beta-galactosidase, green fluorescent protein, and several others.

The determination of activity of membrane-transport proteins can be performed according to techniques such as those described in Gennis, R. B. (1989) “Pores, Channels and Transporters”, in Biomembranes, Molecular Structure and Function, Springer: Heidelberg, p. 85-137; 199-234; and 270-322.

### Example 9

### Analysis of Impact of Mutant Protein on the Production of the Desired Product

The effect of the genetic modification in C. glutamicum on production of a desired compound (such as an amino acid) can be assessed by growing the modified microorganism under suitable conditions (such as those described above) and analyzing the medium and/or the cellular component for increased production of the desired product (i.e., an amino acid). Such analysis techniques are well known to one of ordinary skill in the art, and include spectroscopy, thin layer chromatography, staining methods of various kinds, enzymatic and microbiological methods, and analytical chromatography such as high performance liquid chromatography (see, for example, Ullman, Encyclopedia of Industrial Chemistry, vol. A2, p. 89-90 and p. 443-613, VCH: Weinheim (1985); Fallon, A. et al., (1987) “Applications of HPLC in Biochemistry” in: Laboratory Techniques in Biochemistry and Molecular Biology, vol. 17; Rehm et al. (1993) Biotechnology, vol. 3, Chapter III: “Product recovery and purification”, page 469-714, VCH: Weinheim; Belter, P. A. et al. (1988) Bioseparations: downstream processing for biotechnology, John Wiley and Sons; Kennedy, J. F. and Cabral, J. M. S. (1992) Recovery processes for biological materials, John Wiley and Sons; Shaeiwitz, J. A. and Henry, J. D. (1988) Biochemical separations, in: Ulmann's Encyclopedia of Industrial Chemistry, vol. B3, Chapter 11, page 1-27, VCH: Weinheim; and Dechow, F. J. (1989) Separation and purification techniques in biotechnology, Noyes Publications.)

In addition to the measurement of the final product of fermentation, it is also possible to analyze other components of the metabolic pathways utilized for the production of the desired compound, such as intermediates and side-products, to determine the overall efficiency of production of the compound. Analysis methods include measurements of nutrient levels in the medium (e.g., sugars, hydrocarbons, nitrogen sources, phosphate, and other ions), measurements of biomass composition and growth, analysis of the production of common metabolites of biosynthetic pathways, and measurement of gasses produced during fermentation. Standard methods for these measurements are outlined in Applied Microbial Physiology, A Practical Approach, P. M. Rhodes and P. F. Stanbury, eds., IRL Press, p. 103-129; 131-163; and 165-192 (ISBN: 0199635773) and references cited therein.

### Example 10

### Purification of the Desired Product from C. glutamicum Culture

Recovery of the desired product from the C. glutamicum cells or supernatant of the above-described culture can be performed by various methods well known in the art. If the desired product is not secreted from the cells, the cells can be harvested from the culture by low-speed centrifugation, the cells can be lysed by standard techniques, such as mechanical force or sonication. The cellular debris is removed by centrifugation, and the supernatant fraction containing the soluble proteins is retained for further purification of the desired compound. If the product is secreted from the C. glutamicum cells, then the cells are removed from the culture by low-speed centrifugation, and the supernate fraction is retained for further purification.

The supernatant fraction from either purification method is subjected to chromatography with a suitable resin, in which the desired molecule is either retained on a chromatography resin while many of the impurities in the sample are not, or where the impurities are retained by the resin while the sample is not. Such chromatography steps may be repeated as necessary, using the same or different chromatography resins. One of ordinary skill in the art would be well-versed in the selection of appropriate chromatography resins and in their most efficacious application for a particular molecule to be purified. The purified product may be concentrated by filtration or ultrafiltration, and stored at a temperature at which the stability of the product is maximized.

There are a wide array of purification methods known to the art and the preceding method of purification is not meant to be limiting. Such purification techniques are described, for example, in Bailey, J. E. & Ollis, D. F. Biochemical Engineering Fundamentals, McGraw-Hill: New York (1986).

The identity and purity of the isolated compounds may be assessed by techniques standard in the art. These include high-performance liquid chromatography (HPLC), spectroscopic methods, staining methods, thin layer chromatography, NIRS, enzymatic assay, or microbiologically. Such analysis methods are reviewed in: Patek et al. (1994) Appl. Environ. Microbiol. 60: 133-140; Malakhova et al. (1996) Biotekhnologiya 11: 27-32; and Schmidt et al. (1998) Bioprocess Engineer. 19: 67-70. Ulmann's Encyclopedia of Industrial Chemistry, (1996) vol. A27, VCH: Weinheim, p. 89-90, p. 521-540, p. 540-547, p. 559-566, 575-581 and p. 581-587; Michal, G. (1999) Biochemical Pathways: An Atlas of Biochemistry and Molecular Biology, John Wiley and Sons; Fallon, A. et al. (1987) Applications of HPLC in Biochemistry in: Laboratory Techniques in Biochemistry and Molecular Biology, vol. 17.

### Example 11

### Analysis of the Gene Sequences of the Invention

The comparison of sequences and determination of percent homology between two sequences are art-known techniques, and can be accomplished using a mathematical algorithm, such as the algorithm of Karlin and Altschul (1990) Proc. Natl. Acad. Sci. USA 87: 2264-68, modified as in Karlin and Altschul (1993) Proc. Natl. Acad. Sci. USA 90: 5873-77. Such an algorithm is incorporated into the NBLAST and XBLAST programs (version 2.0) of Altschul, et al. (1990) J. Mol. Biol. 215: 403-10. BLAST nucleotide searches can be performed with the NBLAST program, score=100, wordlength=12 to obtain nucleotide sequences homologous to MCT nucleic acid molecules of the invention. BLAST protein searches can be performed with the XBLAST program, score=50, wordlength=3 to obtain amino acid sequences homologous to MCT protein molecules of the invention. To obtain gapped alignments for comparison purposes, Gapped BLAST can be utilized as described in Altschul et al., (1997) Nucleic Acids Res. 25(17): 3389-3402. When utilizing BLAST and Gapped BLAST programs, one of ordinary skill in the art will know how to optimize the parameters of the program (e.g., XBLAST and NBLAST) for the specific sequence being analyzed.

Another example of a mathematical algorithm utilized for the comparison of sequences is the algorithm of Meyers and Miller ((1988) Comput. Appl. Biosci. 4: 11-17). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art, and include ADVANCE and ADAM. described in Torelli and Robotti (1994) Comput. Appl. Biosci. 10: 3-5; and FASTA, described in Pearson and Lipman (1988) P.N.A.S. 85: 2444-8.

The percent homology between two amino acid sequences can also be accomplished using the GAP program in the GCG software package (available at http://www.gcg.com), using either a Blosum 62 matrix or a PAM250 matrix, and a gap weight of 12, 10, 8, 6, or 4 and a length weight of 2, 3, or 4. The percent homology between two nucleic acid sequences can be accomplished using the GAP program in the GCG software package, using standard parameters, such as a gap weight of 50 and a length weight of 3.

A comparative analysis of the gene sequences of the invention with those present in Genbank has been performed using techniques known in the art (see, e.g., Bexevanis and Ouellette, eds. (1998) Bioinformatics: A Practical Guide to the Analysis of Genes and Proteins. John Wiley and Sons: New York). The gene sequences of the invention were compared to genes present in Genbank in a three-step process. In a first step, a BLASTN analysis (e.g., a local alignment analysis) was performed for each of the sequences of the invention against the nucleotide sequences present in Genbank, and the top 500 hits were retained for further analysis. A subsequent FASTA search (e.g., a combined local and global alignment analysis, in which limited regions of the sequences are aligned) was performed on these 500 hits. Each gene sequence of the invention was subsequently globally aligned to each of the top three FASTA hits, using the GAP program in the GCG software package (using standard parameters). In order to obtain correct results, the length of the sequences extracted from Genbank were adjusted to the length of the query sequences by methods well-known in the art. The results of this analysis are set forth in Table 4. The resulting data is identical to that which would have been obtained had a GAP (global) analysis alone been performed on each of the genes of the invention in comparison with each of the references in Genbank, but required significantly reduced computational time as compared to such a database-wide GAP (global) analysis. Sequences of the invention for which no alignments above the cutoff values were obtained are indicated on Table 4 by the absence of alignment information. It will further be understood by one of ordinary skill in the art that the GAP alignment homology percentages set forth in Table 4 under the heading “% homology (GAP)” are listed in the European numerical format, wherein a ‘,’ represents a decimal point. For example, a value of “40,345” in this column represents “40.345%”.

### Example 12

### Construction and Operation of DNA Microarrays

The sequences of the invention may additionally be used in the construction and application of DNA microarrays (the design, methodology, and uses of DNA arrays are well known in the art, and are described, for example, in Schena, M. et al. (1995) Science 270: 467-470; Wodicka, L. et al. (1997) Nature Biotechnology 15: 1359-1367; DeSaizieu, A. et al. (1998) Nature Biotechnology 16: 45-48; and DeRisi, J. L. et al. (1997) Science 278: 680-686).

DNA microarrays are solid or flexible supports consisting of nitrocellulose, nylon, glass, silicone, or other materials. Nucleic acid molecules may be attached to the surface in an ordered manner. After appropriate labeling, other nucleic acids or nucleic acid mixtures can be hybridized to the immobilized nucleic acid molecules, and the label may be used to monitor and measure the individual signal intensities of the hybridized molecules at defined regions. This methodology allows the simultaneous quantification of the relative or absolute amount of all or selected nucleic acids in the applied nucleic acid sample or mixture. DNA microarrays, therefore, permit an analysis of the expression of multiple (as many as 6800 or more) nucleic acids in parallel (see, e.g., Schena, M. (1996) BioEssays 18(5): 427-431).

The sequences of the invention may be used to design oligonucleotide primers which are able to amplify defined regions of one or more C. glutamicum genes by a nucleic acid amplification reaction such as the polymerase chain reaction. The choice and design of the 5′ or 3′ oligonucleotide primers or of appropriate linkers allows the covalent attachment of the resulting PCR products to the surface of a support medium described above (and also described, for example, Schena, M. et al. (1995) Science 270: 467-470).

Nucleic acid microarrays may also be constructed by in situ oligonucleotide synthesis as described by Wodicka, L. et al. (1997) Nature Biotechnology 15: 1359-1367. By photolithographic methods, precisely defined regions of the matrix are exposed to light. Protective groups which are photolabile are thereby activated and undergo nucleotide addition, whereas regions that are masked from light do not undergo any modification. Subsequent cycles of protection and light activation permit the synthesis of different oligonucleotides at defined positions. Small, defined regions of the genes of the invention may be synthesized on microarrays by solid phase oligonucleotide synthesis.

The nucleic acid molecules of the invention present in a sample or mixture of nucleotides may be hybridized to the microarrays. These nucleic acid molecules can be labeled according to standard methods. In brief, nucleic acid molecules (e.g., mRNA molecules or DNA molecules) are labeled by the incorporation of isotopically or fluorescently labeled nucleotides, e.g., during reverse transcription or DNA synthesis. Hybridization of labeled nucleic acids to microarrays is described (e.g., in Schena, M. et al. (1995) supra; Wodicka, L. et al. (1997), supra; and DeSaizieu A. et al. (1998), supra). The detection and quantification of the hybridized molecule are tailored to the specific incorporated label. Radioactive labels can be detected, for example, as described in Schena, M. et al. (1995) supra) and fluorescent labels may be detected, for example, by the method of Shalon et al. (1996) Genome Research 6: 639-645).

The application of the sequences of the invention to DNA microarray technology, as described above, permits comparative analyses of different strains of C. glutamicum or other Corynebacteria. For example, studies of inter-strain variations based on individual transcript profiles and the identification of genes that are important for specific and/or desired strain properties such as pathogenicity, productivity and stress tolerance are facilitated by nucleic acid array methodologies. Also, comparisons of the profile of expression of genes of the invention during the course of a fermentation reaction are possible using nucleic acid array technology.

### Example 13

### Analysis of the Dynamics of Cellular Protein Populations (Proteomics)

The genes, compositions, and methods of the invention may be applied to study the interactions and dynamics of populations of proteins, termed ‘proteomics’. Protein populations of interest include, but are not limited to, the total protein population of C. glutamicum (e.g., in comparison with the protein populations of other organisms), those proteins which are active under specific environmental or metabolic conditions (e.g., during fermentation, at high or low temperature, or at high or low pH), or those proteins which are active during specific phases of growth and development.

Protein populations can be analyzed by various well-known techniques, such as gel electrophoresis. Cellular proteins may be obtained, for example, by lysis or extraction, and may be separated from one another using a variety of electrophoretic techniques. Sodium dodecyl sulfate polyacrylamide gel electrophoresis (SDS-PAGE) separates proteins largely on the basis of their molecular weight. Isoelectric focusing polyacrylamide gel electrophoresis (IEF-PAGE) separates proteins by their isoelectric point (which reflects not only the amino acid sequence but also posttranslational modifications of the protein). Another, more preferred method of protein analysis is the consecutive combination of both IEF-PAGE and SDS-PAGE, known as 2-D-gel electrophoresis (described, for example, in Hermann et al. (1998) Electrophoresis 19: 3217-3221; Fountoulakis et al. (1998) Electrophoresis 19: 1193-1202; Langen et al. (1997) Electrophoresis 18: 1184-1192; Antelmann et al. (1997) Electrophoresis 18: 1451-1463). Other separation techniques may also be utilized for protein separation, such as capillary gel electrophoresis; such techniques are well known in the art.

Proteins separated by these methodologies can be visualized by standard techniques, such as by staining or labeling. Suitable stains are known in the art, and include Coomassie Brilliant Blue, silver stain, or fluorescent dyes such as Sypro Ruby (Molecular Probes). The inclusion of radioactively labeled amino acids or other protein precursors (e.g., ³⁵S-methionine, ³⁵S-cysteine, ¹⁴C-labelled amino acids, ¹⁵N-amino acids, ¹⁵NO₃ or ¹⁵NH₄⁺ or ¹³C-labelled amino acids) in the medium of C. glutamicum permits the labeling of proteins from these cells prior to their separation. Similarly, fluorescent labels may be employed. These labeled proteins can be extracted, isolated and separated according to the previously described techniques.

Proteins visualized by these techniques can be further analyzed by measuring the amount of dye or label used. The amount of a given protein can be determined quantitatively using, for example, optical methods and can be compared to the amount of other proteins in the same gel or in other gels. Comparisons of proteins on gels can be made, for example, by optical comparison, by spectroscopy, by image scanning and analysis of gels, or through the use of photographic films and screens. Such techniques are well-known in the art.

To determine the identity of any given protein, direct sequencing or other standard techniques may be employed. For example, N- and/or C-terminal amino acid sequencing (such as Edman degradation) may be used, as may mass spectrometry (in particular MALDI or ESI techniques (see, e.g., Langen et al. (1997) Electrophoresis 18: 1184-1192)). The protein sequences provided herein can be used for the identification of C. glutamicum proteins by these techniques.

The information obtained by these methods can be used to compare patterns of protein presence, activity, or modification between different samples from various biological conditions (e.g., different organisms, time points of fermentation, media conditions, or different biotopes, among others). Data obtained from such experiments alone, or in combination with other techniques, can be used for various applications, such as to compare the behavior of various organisms in a given (e.g., metabolic) situation, to increase the productivity of strains which produce fine chemicals or to increase the efficiency of the production of fine chemicals.

Equivalents

Those of ordinary skill in the art will recognize, or will be able to ascertain using no more than routine experimentation, many equivalents to the specific embodiments of the invention described herein. Such equivalents are intended to be encompassed by the following claims.

### Appendix B: Amino Acid Sequences

| > RXA00001 (1-1128, translated) 376 residues | | | | | |
|---|---|---|---|---|---|
| MATVTFKDAS | LSYPGAKEPT | VKKFNLEIAD | GEFLVLVGPS | GCGKSTTLRM | LAGLENVTDG |
| AIFIGDKDVT | HVAPRDRDIA | MVFQNYALYP | HMTVGENMGF | ALKIAGKSQD | EINKRVDEAA |
| ATLGLTEFLE | RKPKALSGGQ | RQRVAMGRAI | VRNPQVFLMD | EPLSNLDAKL | RVQTRTQIAA |
| LQRKLGVTTV | YVTHDQTEAL | TMGDRIAVLK | DGYLQQVGAP | RELYDRPANV | FVAGFIGSPA |
| MNLGTESVKD | GDATSGHARI | KLSPETLAAM | TPEDNGRITI | GFRPEALEII | PEGESTDLSI |
| PIKLDFVEEL | GSDSELYGKL | VGEGDLGSSS | EDVPESGQIV | VRAAPNAAPA | PGSVFHARIV |
| EGGQHNFSAS | TGKRLP | | | | |
| > RXA00002 (1-684, translated) 228 residues | | | | | |
| VLHREGKGGL | LGAYIAGFEW | GLEKDYHVLC | EMDADGSHAP | EQLHLLLEEI | EKGADLVIGS |
| RYVPGGETVN | WPANRELLSR | LGNKYISVAL | GAGINDMTAG | YRAERRELLE | HLDFEELSNA |
| GYIFQVDVAF | RAIKDGEDVR | EVPITFTERE | LGESKLDGSF | VKDSLLEVTK | WGVAHRSEQI |
| SDFTSEVSKI | ASRTVKDMEL | GPKATTAKNA | VPDFVSEVSN | LAKGTFKK | |
| > RXA00089 (1-999, translated) 333 residues | | | | | |
| MATPASAPTS | EPRLKRTRAK | LEDWKLLIGI | IFVAGLVVLS | LLTGQYDIFG | GDDGQLMFEA |
| VRIPRTVSLI | LSGAAMAMCG | LVMQLLTQNK | FVEPSTTGTT | EWAGLGLLFV | IYFVPAATVL |
| DRMLGAVVFS | FIGTMVFFLF | LRRVTLRSSL | IVPIIGIMLG | AVVSSISSFF | ALQFDMLQQL |
| GTWFAGSFNT | VFRGQYEVLW | IVVIVVIAVF | FFADRLTVAG | LGEEIATNVG | LNYNRMVLIG |
| TGLIAIATGV | VTVVVGSLPF | LGLIVPNVVS | MFRGDDLRSN | LPWVCLTGIA | IVTICDLISR |
| TIIAPFEIPV | SVILGIIGAV | VFVIMIVRQR | GRG | | |
| > RXA00090 (1-1119, translated) 373 residues | | | | | |
| VAVDKDIENR | TSDLSRWETM | EESATVEGRT | DVELASAPSK | RRTSGAFQTA | RAKRRYWIIM |
| AALLVTALAF | TWGLIWYKNP | MPVGHPAFAL | IAERRMESVF | VMLIVAVCQG | FATVAFQTVT |
| NNRIITPSIM | GFESLYTLIH | TSTVFFFGAT | ALLATRNLEM | FVGQLVIMVL | LTLVLYTWLL |
| SGKRGDMHAM | LLVGIIIGGG | LGSISTFMQR | ILTPSEFDIL | SARLFGSVNN | AETEYFPIAV |
| PLVVVASVLL | LLSSRRLNVV | GLGKDAATNL | GINHRRSSIY | TLVLVSVLMA | VSTALVGPMT |
| FLGELVATLA | YQFADTYDHR | YILPMSALIG | FVVLSGAYFV | MNHVFRAQGV | VSIIIEMVGG |
| TVFLIVILRK | GRL | | | | |
| > RXA00099 (1-1173, translated) 391 residues | | | | | |
| VKNPRLIALA | AIILTSFNLR | TAITALAPLV | SEIRDDLGVS | ASLIGVLGMI | PTAMFADAAF |
| ALPSLKRKFT | TSQLLMFAML | LTAAGQIIRV | AGPASLLMVG | TVFAMFAIGV | TNVLLPIAVR |
| EYFPRHVGGM | STTYLVSFQI | VQALAPTLAV | PISQWATHVG | LTGWRVSLGS | WALLGLVAAI |
| SWIPLLSLQG | ARVVAAPSKV | SLPVWKSSVG | VGLGLMFGFT | SFATYILMGF | MPQMVGDPQL |
| GAVLLGWWSI | LGLPLNILGP | WLVTRFTNCF | PMVVIASVMF | LIGNGGFCLA | PDVAPWLWAT |
| LSGLGPLAFP | MALTLINIRA | ETSAGASALS | SFGQGLGYTI | ACFGPLLTGF | IVDATGSFRT |
| IFLLFAGATL | FVTRGGYFAT | RQVYVEKLLN | R | | |
| > RXA00123 (1-1119, translated) 373 residues | | | | | |
| MPKNYDINGA | IRRRDMLRRR | YLPDSANSTP | VPEEVSPLTR | YVTDGIPKRP | PLGATVADGL |
| KFAEGASNRM | VMSLYPAPSK | PAIEELAEAW | DLHPTIVEDL | LLGQQRPKLD | RYEDIIFIAI |
| RSARYIDSRE | EVDFSEFHIL | MKPQAIAILC | QDNQWIDGTS | AASFSNPEEI | DKRIKTLLAD |
| AELLSSGPRA | AAYRLLDAIV | DGFSPVLRGI | AIDQEQIERQ | VFSGDAAVAE | RIYNLSQEII |
| DMQHTTSSVT | EVVQRLNKDF | IRSGMSEELR | AYLDDVADHL | TRDNTRVSEY | RESLSQILNV |
| NATLVAQRQN | EDMKKISGWA | AIIFAPTLVS | SIYGMNFDIM | PELHWAFGYP | LALLANLGFT |
| LLLYWIFKRS | KWM | | | | |
| > RXA00160 (1-573, translated) 191 residues | | | | | |
| MLNIARNRNM | KRRLAIAAFV | ATATATATMA | PASAQTDYAG | LSSGVADTVA | EAAGVATTAV |
| APAATVARPA | NGTFTSGFGP | RWGTFHNGID | IANSIGTPIY | AVMAGTVISS | GPASGYGQWI |
| RIQHDDGSIS | IYGHMEYLYV | SVGERVAAGQ | EIAGMGSQGF | STGSHLHFEI | HPDGVTPVDP |
| QAWLANHGIY | V | | | | |
| > RXA00193 (1-843, translated) 281 residues | | | | | |
| MQATLKKYFP | VFVLPTLLAF | MIAFLVPFIV | GFFLSFTKFT | TITNAKWVGI | DNYVKAFSQR |
| EGFISAFGFT | VLVVIVSVIT | VNIFAFLLAW | LLTRKLRGTN | FFRTVFFMPN | LIGGIVLGYT |
| WQTMINAVLS | HYATTISADW | KFGYAGLIML | LNWQLIGYMM | IIYIAGLQNV | PPELIEAAEL |
| DGVNKWEMLR | HVTIPMVMPS | ITICLFLTLS | NSFKLFDQNL | ALTNGAPGGQ | TEMVALNIIN |
| TLFNRMNVEG | VGQAKAVIFV | VVVVVIAYFQ | LRATRSKEIE | A | |
| > RXA00203 (1-912, translated) 304 residues | | | | | |
| MLNNGALVGL | IALCVGLFIA | TPHFLTIPNL | INIGIQSATV | AILAFGMTFV | IVTAGIDLSV |
| GSVAALGAMT | SAYFFAEVGL | PGWITLLIGL | FIGLLAGAIS | GISIAYGKLP | AFIATLAMMS |
| IARGITLVIS | QGSPIPSAPA | VNALGRTYFG | IPMPILMMAL | AGIVCWFILS | RTVLGRSMYA |
| IGGNMEAARL | SGLPVKKILV | MVYALAGVYA | ALAGLVMTGR | LSSAQPQAGV | GYELDAIAAV |
| VIGGASLAGG | TGKATGTLIG | AILLAVIRNG | LNILNVSSFW | QQIVIGCVIA | LAVGFDVIRN |
| KTSR | | | | | |
| > RXA00204 (1-1572, translated) 524 residues | | | | | |
| MVNSEQALHQ | HDPAPILQLD | KVSKSFGPVN | VINQVSIDVR | PGRVLALLGE | NGAGKSTLIK |
| MMSGVYQPDG | GQILVDGKPT | TLPDTKTAES | FGIATIHQEL | NLVPTMTVAE | NVMLGRTPRK |
| WGLVNFKHLR | RQAQAALDLI | GVDVDLNAQV | GSLGIARQQM | VEIAKALSMN | ARILILDEPT |
| AALTGREIDQ | LFKVVDQLKE | KGVAMVFISH | HLDEIARIGD | TVSVLRDGQF | IAELPADTDE |
| DELVRLMVGR | SIENQYPRSA | PEIGQPLLEV | KNLNAEGRFT | DISLTVRAGE | VVGLAGLVGA |
| GRTEVVRSIA | GVDKVDSGEV | IVAGKKLRGG | DISEAIKNGI | GHIPEDRKAQ | GLVLGSSVED |
| NLGLATLAST | ARAGLVDRSG | QHKRAAEVAE | KLRIRMASLK | QPISDLSGGN | QQKAVFGRWV |
| LAGSNVLLLD | EPTRGVDVGA | KVEIYNIINE | MTEKGGAVLM | VSSELPEVLG | MADRILVMSG |
| GRIAGELPAK | GTTQDDVMAL | AVSQVDDSIT | EEAAAEIENT | KEDR | |
| > RXA00270 (1-888, translated) 296 residues | | | | | |
| MIGAFEFGLL | YGVVALGVYL | TFRVLNFPDL | TVDGSLTTGA | ATAATALMSG | WPPLMATAAG |
| FVTGFIAGMI | TGLLHTKGKI | DGLLAGILTM | IALWSVNLRI | MGGANVPLLR | TDNLFTPLRD |
| AGLLGTWAGP | AILAVAVGIL | GLIVIWFLNT | DIGLSLRSTG | DNGPMVQSFG | VSTDFTKILT |
| ISLSNGFVGL | AGALIAQYQG | FADISMGIGL | IVIGLASVIL | GQAIFGQRRV | WLAVLAVIVG |
| AIAYRLIIFA | ALRVGLDPND | MKAISAILVV | VAMLLPRWRA | KFSKAPKPKQ | PVAVEA |
| > RXA00311 (1-855, translated) 285 residues | | | | | |
| MEHSPEGKRG | FFTSSVMAGC | SVGNVLAGLV | FIPFLMLPEE | HLMSWGWRVP | FLLSALVLVV |
| AYFVRTRLEE | ASTEKAEEDA | GAPALAVLRT | QGIDVARVFL | ITFFAVVQTT | FNVYALAYAA |
| NEIGIDRSFM | VMVNTIALGL | SIGTIPLAAW | VSDRIGRKPV | LLFGAITCAI | TTYFYFQAIS |
| EADLVLIFAL | CLVNQGLFYS | CWNGVWTIFF | PEMFASSVRY | TGMAMGNQLG | LIIVGFAPTI |
| ATALYAWNGW | EAVAGFIIGA | IALSAAVILT | TKETAFTKLE | DLGKK | |
| > RXA00312 (1-426, translated) 142 residues | | | | | |
| METVRTATAA | PETASLKLRE | AESPAKSPKK | AALASLLGST | LEYYDFVIYG | TASALLFNHL |
| FFPQGDPVVA | TIGSLASFGV | AYIARPIGGL | VMGHVGDKIS | RKTALMVTLM | IMGIASISIG |
| LLPTYGQIGI | WATVLLMIAR | IA | | | |
| > RXA00345 (1-951, translated) 317 residues | | | | | |
| MAGMKKLLWT | LPILPLVLAG | CSTGSADSAD | STNAAGSNSL | KVVTSTQVWA | DVAEAVAPDV |
| DIEAIITGGD | IDPHSFEPSA | TDMAKVSEAD | IIIVGGGGYD | SWLYGTLEDD | DRIIHALDLS |
| EHDHSEHDDH | EHEAEEAHEH | DHDEEGHDHD | VDNEHVWYST | EYVSEVAEEF | AEKVTELDPE |
| AQADATAVTT | KMDELHNQIH | DLPAVRIAQT | EPIADHILSH | SDMVESTPEG | YRATTLSESE |
| PTAADVASFQ | DAINNGDLDV | LTYNPQSAST | VATSLKDLAE | EKGIPVVEIY | ETPQNTENFL |
| DAFTKAVDDL | TAATNQV | | | | |
| > RXA00378 (1-1773, translated) 591 residues | | | | | |
| KSWRSYPSWF | AFDHGTLTQN | EIYFDVACGI | TVLLLAGRLL | TRRRSQSSLL | AELGRLQIDP |
| QRIVTVVRKH | RLKRVVQELN | IPVQEVRVND | DVKVPPNTTI | PVDGTVIGGG | SRIAASIIMG |
| QDQRDVKVND | KVFAGSLNLE | SEIKVRVIRT | GHRTRIAAVH | RWVKEATLKE | NRHNRAAIRS |
| AGNLVPITFT | LAVVDFCLWA | LISGNINAAF | TTTLAVLACV | APVALALSAP | LATRNSIEAA |
| ARHGILVRSG | EIFRVLDDVD | TAVFNRVGTL | TDGEMTVETV | TADKGEDPEL | VLRVAGALAM |
| ESHHAISKAL | VKASREARDT | GAGGEDVPHW | IEVGNVEITE | AGSFQATIEL | PLIKPSGEKI |
| MRTTEALLWR | PRSMTEVREH | LSPRLVAAAT | SGGAPLIVRW | KGKDRGVITL | SDHVRSDSSD |
| AIIAIEEQGI | ETMMLSRDTY | PVARRYADSL | GITHVLAGIA | PGKKAQVVRA | VHTRGSTVAM |
| IGDESVMDCL | KVADVGVLMG | VDRPSDLRDD | SDDPAADVVV | MREEVMSVPT | LFKLARRYAK |
| LVNGNIALAW | IYNGVAMVLA | VSGLLHPMAA | TVAMLASSLL | IEWRSGRARK | Y |
| > RXA00412 (1-1080, translated) 360 residues | | | | | |
| VSHTASTPTP | EEYSAQQPST | QGTRVEFRGI | TKVFSNNKSA | KTTALDNVTL | TVEPGEVIGI |
| IGYSGAGKST | LVRLINGLDS | PTSGSLLLNG | TDIVGMPESK | LRKLRSNIGM | IFQQFNLFQS |
| RTAAGNVEYP | LEVAKMDKAA | RKARVQEMLE | FVGLGDKGKN | YPEQLSGGQK | QRVGIARALA |
| TNPTLLLADE | ATSALDPETT | HEVLELLRKV | NRELGITIVV | ITHEMEVVRS | IADKVAVMES |
| GKVVEYGSVY | EVFSNPQTQV | AQKFVATALR | NTPDQVESED | LLSHEGRLFT | IDLTETSGFF |
| AATARAAEQG | AFVNIVHGGV | TTLQRQSFGK | MTVRLTGNTA | AIEEFYQTLT | KTTTIKEITR |
| > RXA00413 (1-897, translated) 299 residues | | | | | |
| MKLRRITTTA | IAGLFAATAL | VACGSDSDGS | STTVAEGTEG | VTIRIGTTDA | AKEAWTVFED |
| KAAEEGITLD | IVPFSDYSTP | NEALAQDQLD | VNLFQHLKFL | AEYNVGSGAD | LTPVGSSEIV |
| PLALFWKDHD | SIDGIDGESV | AIPNDPSNQG | RAINVLVQAG | LVTLKTPGLV | TPAPVDIDEA |
| ASKVSVIPVD | AAQAPTAYQE | GRPAIINNSF | LDRAGIDPNL | AVFEDDPESE | EAEPYINVFV |
| TKAEDKDDAN | IARLVELWHD | PEVLAAVDRD | SEGTSVPVDR | PGADLQEILD | RLEADQENA |
| > RXA00431 (1-675, translated) 225 residues | | | | | |
| MVSIDTYNAC | VDFPIFDAKS | RSMKKAFLGA | AGGAIGRNQD | NVVVVEALKN | VNLHLREGDR |
| VGLVGHNGAG | KSTLLRLLSG | IYEPTRGSAD | IRGRVAPVFD | LGVGMDPEIS | GYENIIIRGL |
| FLGQTRKQMK | AKMEEIADFT | ELGEYLSMPL | RTYSTGMRIR | LALGVVTSIE | PEILLLDEGI |
| GAVDAAFMAK | ARDRLQALVE | RSGILVEAST | QRLSCQLCNT | ALWVD | |
| > RXA00444 (1-777, translated) 259 residues | | | | | |
| LLIPATLAML | LIIGPIEALL | LQIPWDRSWE | LLTAPESLGT | ARLSIGTALF | STALCAIVGF |
| PLALALHLYE | RSHPRVTSVL | TVLVYAPLVL | SPVVSGLALT | FLWGRRGFLG | SWLDQVGLPI |
| AFTTTAVVFA | QVFVALPFFI | STVTTALRGI | PKQFEEIAAT | EGATRWEIMH | KMIIPLAMPG |
| IFTGMILGFA | RALGEYGATL | TFAGNIAGVT | RTIPLHIELG | LSSNDMDKAL | GAVIMLLAVY |
| VLIIGAIGAL | RLESKVRKV | | | | |
| > RXA00445 (1-912, translated) 304 residues | | | | | |
| MADLSIEHVS | RFFGDAIALN | DVSLTVPSGS | ITAIIGPSGS | GKTTLLRLLA | GLDSPDEGTV |
| SIGNKIAKLG | DTALCEQDSP | LYPHLNVWEN | VAFPLKLKAT | NTADEVVKKR | VSDVLEMLEI |
| APLARRKITE | LSGGQKQRVG | IARALVRDVE | VYLFDEPMAH | LDQALARDIV | ADLRKIQQSL |
| GLTFVYVTHS | KSEAFALADQ | IVVLVDGQVA | QVGEAEELVE | KPKTLEIAEF | LSPTELNVRR |
| RGDAVEAWRP | EDTQLARGGT | ATVEAVTYLG | REWLVQTTEG | HAVSEEKEDV | GESVTLTQKK |
| VESF | | | | | |
| > RXA00466 (1-987, translated) 329 residues | | | | | |
| VQSRLSKILR | SSVVGVAVLA | LLAGCSNNAD | DTDADSTSTG | NSAFPVSIEH | EFGTTTIDDV |
| PERVVTLGVT | DADIVLALGT | VPVGNTGYKF | FENGLGPWTD | ELVEGKELTL | LDSDSTPDLE |
| QVAALEPDLI | IGVSAGFDDV | VYEQLSDIAP | VVARPAGTAA | YAVAREEATN | LVARAMGQSE |
| KGQELNEETD | ALIQAARDEN | PSFDGKTGTV | ILPYQGKYGA | YLPGDARGQF | LDSLGISLPE |
| AVLSRDTGDS | FFVDVPAESV | KDVDGDVLLV | LSNDENLDIT | AENPLFETLN | VVQKDAVIVA |
| TTEERGAITY | NSVLSVPFAL | EHLAPRIAE | | | |
| > RXA00482 (1-648, translated) 216 residues | | | | | |
| MRISSKLVTT | ALLAAISLFG | ISTAQAQDIF | DGGRLAGGSS | QVSNLSSVPE | NLALPEIENS |
| IDLERYKGKW | YQVAAIPQPF | SLQCSHDVTA | DYGVIDSDTI | SVTNKCGTFF | GPSVIEGSAK |
| VVSNASLKVS | FPGIPFQSED | NQANYRVTYI | EDDYSLAIVG | SPSRSSGFIL | SRTPQLSSDQ |
| WSHVRNITED | SGWWPCAFIT | VPATGGLNTA | TPLCTL | | |
| > RXA00523 (1-750, translated) 250 residues | | | | | |
| VLRNQLASPD | IIGISSGASA | AGVICIVFFG | MSQSAVSAIS | LCASLAVALL | IYLVAYRGGF |
| SATRLILTGI | GIAAMLNSLV | SYSLSKADSW | DLPTATRWLT | GSLNGATWDR | AMPLIVTTVV |
| LIPLLVANAR | NVDLMRLGND | SAVGLGVATN | RTRVIAIIAA | VALIAVATAA | CGPIAFVAFV |
| SGPIAARILG | SGGSLIIPSA | LIGGLIVLIA | DLIGQYFLGT | RYPVGVVTGA | FGAPFLIYLL |
| IRSNRAGVTL | | | | | |
| > RXA00525 (1-660, translated) 220 residues | | | | | |
| MSLAESILLA | LTSLRSNKMR | ALLTLLGVII | GIASVIGILT | IGKALQDQTL | NSLESLGAND |
| LSAQVEERPD | EDSPEPDMFA | FSGAANSSGN | LIPEETVDTL | RDRFAGSITG | ISVGGMGTQG |
| TLIGDTADLK | SDLLGVNEDY | MWMNGVEMNY | GRAITQDDVA | AQRPVAVIAP | DTFNTLFDAN |
| PNLALGSEVA | FELNGQETFL | RVIGVYKEAA | AGGLVGSNPT | | |
| > RXA00556 (1-594, translated) 198 residues | | | | | |
| YTPYTVANDI | THTKDGLNTL | SIRAAQGVDQ | DSLKGSLQTY | FDALYANNDS | HHVAMLDFRK |
| QIEEFNTILG | AMSLGISAIG | GISLLVGGIG | VMNIMLVSVT | ERTREIGVRK | ALGARRRDIR |
| LQFVVEAMII | CFIGGILGVL | LGGILGLIMS | SAIGYISLPP | LSGIVIALVF | SMAIGLFFGY |
| YPANKAAKLD | PIDALRYE | | | | |
| > RXA00596 (1-453, translated) 151 residues | | | | | |
| MLNALKFIPW | LIGQIFLSGF | SVITAAVKKD | TGFNPVVIRY | PLRVTTDFQI | AALSTCITAT |
| PSTLSLGLRE | PRKPGDPTIL | LIQAVFGSDP | VEVFESIADM | EQRLVPSVAS | IDHGVPGQGP |
| YKEIRPSDAE | WPSREIADTA | QNTVSQDKRE | F | | |
| > RXA00634 (1-1383, translated) 461 residues | | | | | |
| MWERFSFYGM | QALLVYYLYF | DVAAGGLGLD | QTQATGLVGV | YGALLYLCCW | AGGWVSDRVL |
| GAEKTLLGGA | ISVTIGHLVL | AGLGGKIGLA | IGLGCIAIGS | GFVKTAAITV | LGSRHGEQEG |
| DAKADPAFQL | FYLGINVGAL | LGPLLTGWLS | SRYSFEMGFG | AAAVLMIGGL | GIYAALRKPM |
| LQSFPLEVKK | ALLRAQNPAE | KHVISTAFAA | VAVLCGVLLY | LLLTETVSAD | QLAGALLLVT |
| IGAALWLIIQ | PLRHPQVSSE | EKRKVLAFIP | IFVCSTAFWA | VQAQTYGVLA | VYSQERVDRM |
| VGDFEIPAAW | SQSLNPFFIL | ALSIPISLWF | MRGSRAPRVK | IGISIGVIIA | GSGLLVLIPF |
| VGMPLAPVWV | LPLSVFLISL | GELFIGPGGM | AATAHHAPRI | FATRFSALYF | LTLAIGMSIA |
| GNVSKFYDPT | NHTSELRYFA | VFGISIIVIG | VGSLMVAKKV | G | |
| > RXA00665 (1-438, translated) 146 residues | | | | | |
| MSSSTLLLAS | GQVTALAADY | TLSHTPSDGI | LVVLGFAMIL | TFMTLIMLGR | LTPMVAMLLV |
| PTIFGLIAGA | GLGLGDMALD | AIKDMAPTAA | LLMFAIMFFG | IMIDVGLFDP | LIRVITRVLH |
| DDPAKVVIGT | AVLAGVVSLD | GDGSTT | | | |
| > RXA00702 (1-1320, translated) 440 residues | | | | | |
| LGLPPAVMRK | RVEETLDLLG | IAELRYVPLA | ELSGGEQQRV | AIGAVLTTRP | ALIILDEPTS |
| ALDPNGAEDV | LATVTKLAHD | LAMTVVLAEH | RIERVLQYVD | RVAHVGADGH | VTVGTPEEIM |
| ADSDVAPPIV | ELGRWAGWAP | LPLSIRDARA | HSADMRKRLY | QRGLVVNKLH | NHAVQPLLIA |
| EDIMVDFPEI | RAVDGVNLNL | NSGEITVLMG | RNGCGKSSLL | WALQGSGTRN | QGSVQVLDEA |
| AGFSWTDPKT | LKPAKRRNLV | SMVPQTPTDI | LYESTVHAEL | ARSDKDAAAP | AGTTREILDS |
| LVPNIPDHLH | PRDLSEGQKL | SLALSIQLAA | KPRVVFFDEP | TRGLDYDGKK | SLARSFQQLA |
| DDGHAILVVT | HDVEFSALCA | DRVLFMASGK | IISDGTAVEI | LPASPAYAPQ | VAKITAGIQE |
| ESHWLTVSAV | KAALGHGEIS | | | | |
| > RXA00728 (1-792, translated) 264 residues | | | | | |
| VAAAIIVALL | AWFIISALNN | EAYGWDTYRS | YLFDTRIATA | ALHTIALTLL | SMILGVVLGA |
| ILAVMRMSGN | PVMQGVAWLY | LWIFRGTPIY | VQLVFWGLLG | SLYQSINLGF | AEIDLQSLLS |
| NMFLLAVIGL | GLNEAAYMAE | IVRSGIQAVP | EGQMEASKAL | GMNWSMTMRR | TILPQAMRII |
| IPPTGNELIS | MLKTTSLVVA | IPYSLELYGR | SMDIAYSLFE | PVPMLLVAAS | WYLVITSILM |
| VGQYYLEKHF | EKGSTRTLTA | RQLA | | | |
| > RXA00732 (1-822, translated) 274 residues | | | | | |
| MLVQMTSTLM | ISAPMLAIGG | IIMAVRQDLG | LSWLMVVSIP | VLIIVVALII | VRMVPLFQTM |
| QKRIDRINQI | IREQLTGIRV | IRAFVREDVE | RERFTTASKD | VADIGVRTGN | LMALMFPAVM |
| LIMNLSAVAV | IWFGAFQVES | GETQIGTLFA | FLQYIMQILM | GVMMAAFMFV | MVPRAAVSAD |
| RIGEVLETTP | SVQAPETPAQ | PSTSAGEIVF | NNATFAYPGA | DDPVLNNVSF | RVAPGSTTAI |
| IGSTGSGKTT | LIGLVPRLFD | VTEGDVTVDG | TDVR | | |
| > RXA00734 (1-453, translated) 151 residues | | | | | |
| RHLRYGNEDA | TETQLWQALA | IAQAADFVRE | MPEGLDSEIA | QGGTNVSGGQ | RQRLAIARAL |
| LKQPEIYIFD | DSFSALDVST | DAALRRALST | NLPDATKLIV | AQRVSTIRDA | DQIVVLDNGE |
| VVGIGTHTNL | LNTCGTYREI | VESQETAQAQ | S | | |
| > RXA00759 (1-924, translated) 308 residues | | | | | |
| MLRYVGRRLL | QMIPVFFGAT | LLIYALVFLM | PGDPVQALGG | DRGLTEAAAE | KIRQEYNLDK |
| PFIVQYLLYI | KGIFVLDFGT | TFSGQPVIDV | MARAFPVTIK | LAIMALLFES | ILGIIFGVIA |
| GIRRGGIFDS | TVLVLSLIVI | AVPTFVIGFV | LQFLXGVKWG | LLPVTVGSNT | SITALIMPAV |
| VLGAVSFAYV | LRLTRQSVSE | NLRADYVRTA | RAKGMSGENV | MNRHVLRNSL | IPVATFLGAD |
| LGALMGGAIV | TEGIEGINGV | GGTLYQAILK | GEPTTVVSIV | TVLVIVYIIA | NLLVDLIYAV |
| LDPRIRYA | | | | | |
| > RXA00760 (1-1032, translated) 344 residues | | | | | |
| MPNNEFHTNH | SLGQDDQTPD | QAHFFPQGRG | EALVRPGQEH | FIAATDETGL | GAVDAVADDS |
| APTSMWGEAW | RDLRRRPLFW | VSAVLIILAL | LLAAVPQLFT | STDPQFCVLA | NSLDGPQSGH |
| PEGFDRQGCD | IFARTVYGAR | ASVAVGVLTT | LLVALIGTVF | GALAGFFGGI | MDTILSRITD |
| MEFAIPLVLA | AIVVMQMFKE | HRTIVTVVLV | LGLFGWTNIA | RITRGAVMTA | KNEEYVTSAR |
| ALGASKAKIL | LSHIMPNAAA | PIIVYATVAL | GTFIVAEATL | SFLGIGLPPS | IVSWGADIAK |
| AQTSLRTQPM | VLFYPAMALA | LTVLSFIMMG | DVVRDALDPK | SRKR | |
| > RXA00761 (1-591, translated) 197 residues | | | | | |
| MTTNIPQTPN | HEGEQPLLEL | KDLKISFTSS | TGVVDAVRGA | NLTIYPGQSV | AIVGESGSGK |
| STTAMSIIGL | LPGTGKVTEG | SIMFDGQDIT | GLSNKQMEKY | RGSEIGLVPQ | DPMTNLNPVW |
| RIGTQVKESL | RANHVVPGSE | MDKRVAEVLA | EAGLPDAERR | AKQYPHEFSG | GMRHRALIAI |
| GLAARPKLLI | ADEPTSA | | | | |
| > RXA00774 (1-654, translated) 218 residues | | | | | |
| MDKATDALLR | TSLASAESAL | GNAEKLEELR | TGCESQAVEL | LALETPVARD | LRQVVSSIYI |
| VEEITRMGAL | AMHVANSVRR | RYPDPVIPED | MRGYFKEMAR | LAADMTDHIR | QILIDPEPDL |
| ALEMAKSDDA | VDDLHQHIMR | ILTLRPWPHD | TKSAVDLTLL | SRFYERYADH | TVNVAARIIY |
| LSTGLHPEEY | MEKREQQRAD | ADMEKRWAEL | ERQFRTSE | | |
| > RXA00775 (1-771, translated) 257 residues | | | | | |
| MSKLKLNDVN | IYYGDFHAVQ | NVNLEVPARS | VTAFIGPSGC | GKSTVLRSIN | RMHEVTPGAY |
| VKGEILLDGE | NIYGSKIDPV | AVRNTIGMVF | QKANPFPTMS | IEDNVVAGLK | LSGEKNKKKL |
| KEVAEKSLRG | ANLWEEVKDR | LDKPGGGLSG | GQQQRLCIAR | AIAVEPEILL | MDEPCSALDP |
| ISTLAVEDLI | HELKEEFTIV | IVTHNMQQAA | RVSDQTAFYS | LEATGRPGRL | VEIGPTKKIF |
| ENPDQKETED | YISGRFG | | | | |
| > RXA00776 (1-921, translated) 307 residues | | | | | |
| MTNNVVTPRM | DEPLKKSSAF | TDISSSRKTT | NTAATVIIYG | AMLIAAVPLV | WVLWTVISRG |
| IAPILTADWW | STSQAGVMLM | LPGGGAAHAM | IGTFMQAVVT | SVISIPIGIF | TAIYLVEYSN |
| GNRLGRLTTF | MVDILTGVPS | IVAALFVYSL | WIVLFGFDRS | GFAVSLSLVI | LMVPVIIRNT |
| EEMLRVVPQD | LREASYALGV | PKWKTIAKIV | LPTALSGIVT | GVMLAVARVM | GESAPVLVLV |
| GSSQAINWNP | FGGPQASLPL | MMLDMYKAGT | APATLDKLWG | AALTLVLIIA | VLNIGARIIS |
| AKFSVKQ | | | | | |
| > RXA00777 (1-1065, translated) 355 residues | | | | | |
| MATNESVSEK | QRLDATRVQA | HPVAVNANSS | QTKPSKKIVA | EGGGSVKRPG | DRIFEVLSTA |
| SAAIITAIII | AIAAFLIWRA | VPALMRNAEG | IGGFFTYSGA | WNTTDIDAMY | FGIPNLLAAT |
| LLISVIALII | AMPIALGIAI | FLSNYSPKRL | VKPLGYMVDM | LAAVPSIVYG | LWGWQVLGPA |
| LSGFYTWIES | WGGSFFLFAT | YQNSPSFATG | RNMLTGGIVL | AVMILPVIEA | TAREVFIQTP |
| KGHIESALAL | GATRWEVVRL | TVLPFGMSGY | VSGAMLGLGR | ALGETMALYM | VVSPSSAFRF |
| SLFDGGTTFA | TAIANAAPEF | NDNTRAGAYI | SAGLVLFALT | FIVNAGARAM | VNRGK |
| > RXA00828 (1-369, translated) 123 residues | | | | | |
| EHQFVARTVR | DELEIGPKIM | KVDASERIEE | LLDRLRLRHL | ENANPFTLSG | GEKRRLSVAT |
| ALVAAPKLLI | LDEPTFGQDP | ETFTELVTML | RELTDNGISI | VSVTHDPDFI | AALGDHHIEV |
| SAK | | | | | |
| > RXA00832 (1-555, translated) 185 residues | | | | | |
| TLTAVVYGFF | LFRQMGAQAG | EFQEVEVAEK | ADDAAKWEVP | FRGLILIITV | LPIVLLSHDM |
| ATVMDEVLAS | LGAPVAMAGL | IIATIVFLPE | TITSLKAAWT | GEIQRVSNLA | HGAQVSTVGL |
| TIPAVLVIGV | ITGQDVVLGE | TPINLLLLGT | TIAVTAIAFS | SKKVSAVHGS | VLLMLFGVYM |
| MSMFA | | | | | |
| > RXA00934 (1-789, translated) 263 residues | | | | | |
| PSFSMAALPF | AEGPIVATYH | ASSSGSKLLK | AFLPVLSPML | EKVRAGIAVS | EMARRWQVEQ |
| VGGDPVLIPN | GVETSMFKAA | RQIEPNDPVE | IVFLGRLDES | RKGLDILLRA | LTRLDRPFTC |
| TVIGGGTPRE | VAGINFVGRV | SDEEKAAILG | RADIYVAPNT | GGESFGIVLV | EAMAAGCAVV |
| ASDLEAFSLV | TDSEAAQPAG | VLFKTGSDAD | LAKKLQALID | DPSSRSTLIA | AGLKRANAYD |
| WSTVSTQVMA | VYETIAIDKV | RLG | | | |
| > RXA00939 (1-168, translated) 56 residues | | | | | |
| GVLLGGVTMS | IGMLVHEASV | LLVIAIAMLL | LRPTLKEDKD | KADVSTADAA | KETLSA |
| > RXA00942 (1-204, translated) 68 residues | | | | | |
| LSTKNYHVEG | LTCANGVASV | EDEIGIVAGT | QGVDIDIETG | RVTVTGEGFT | DEEIIEAVAN |
| AGYKVSGR | | | | | |
| > RXA00950 (1-906, translated) 302 residues | | | | | |
| MNTPAVQVQN | LSLSEGSFTA | VNGLSLTVEQ | GSIHGFLGPN | GAGKSTTIRA | LIGVLKPQTG |
| SVAILGQDPV | AHPDVLRRVG | YVPGDATLWD | NLTGAEVFRA | LESLRKTPSN | RALENELIDA |
| FQLDPSKKIR | EYSTGNRRKV | SLIAALSHEP | ELLIVDEPTA | GLDPIMEQVF | VTYVRKARTN |
| GASVLLSSHI | LSEVEQLCDY | VTVLKEGRAV | ASNEVSYLRK | ISAHRITATI | PAVPQHLAGR |
| GEVDFDAGHL | SITCDASEVP | DILRIIIDAG | GQDIISTAAS | LEEIFLRHYG | ETVSGSESKA |
| SQ | | | | | |
| > RXA00960 (1-459, translated) 153 residues | | | | | |
| LKNDVDVNVA | GFVVPLCATI | HLAGSMMKIG | LFTFAVVFMY | DMEVGVGLSI | GFLLMLGITM |
| IAAPGVPGGA | IMAATGMLAS | MLGFNTEQVA | LMIAAYIAID | SFGTAANVTG | DGAIAVIVNK |
| FAKGQLHTTS | PDEIEEDDRV | AFDITPSDVE | HHK | | |
| > RXA00980 (1-639, translated) 213 residues | | | | | |
| MFVGVNGHAI | GIVAVADAVR | SDSASAIESL | HKAGIQVVMA | TGDAHRVAQN | VASKLGVDEV |
| YSELLPEQKL | ELVRDLQAAG | KTVAMVGDGV | NDTPALAAAD | IGVAMGVAGS | PAAIETADIA |
| LMADRLPRLA | HAVTLAKRTV | RTMRINILIA | LATVMVLLAG | VLFGGVTMSV | GMLVHEASVL |
| LVISIAMLLL | RPTLKEDAAQ | ASDIKRSEIQ | QIA | | |
| > RXA01000 (1-540, translated) 180 residues | | | | | |
| MLAARGVGPY | WLRTVLRFVF | AVIRAFPEVV | IAIILLTVTG | LTPFTGALAL | GISGIGQQAK |
| WTYEAIESTP | TGPSEAVRAA | GGTTPEVLRW | ALWPQVAPSI | ASFALYRFEI | NIRTSAVLGI |
| VGAGGIGSML | ANYTNYRQWD | TVGMLLIVVV | VATMIVDLIS | GTIRRRIMKG | ASDRVVAPSN |
| > RXA01002 (1-417, translated) 139 residues | | | | | |
| PTEHDKQIAF | HALESVGILD | KVWTRAGALS | GGQKQRVAIA | RALSQDPSVM | LADEPVASLD |
| PPTAHSVMRD | LENINNVEGL | TVLVNLHLID | LARQYTTRLV | GLRAGKLVYD | GPISEATDKD |
| FEAIYGRPIQ | AKDLLGDRA | | | | |
| > RXA01003 (1-804, translated) 268 residues | | | | | |
| MTTPSSTLIP | QKPRAGVKTY | LIIGAIVVFT | VATATPALGG | IELDFASIAA | NWRNGANKLL |
| QMLQPNFAFL | PRTWLPMLET | LQMALVGAVL | SAAVSVPLTL | WAAQATNTSA | IGRGIVRTII |
| NVVRSVPDLV | YATILVAMVG | VGALPGILTL | FLFNLGIVVK | LVSEAIDSTE | HPYMEAGRAA |
| GGSQFQINRV | SALPEVMPLF | ANQWLYTLEL | NVRISAILGI | VGAGGIGRLL | DERRAFYAYA |
| DVSVIILEIL | IVVIVIEVIS | NALRKRLV | | | |
| > RXA01006 (1-858, translated) 286 residues | | | | | |
| MTTSQILRRI | GQAVLVLLVT | FTLAFIMLSA | LPGDAVSARY | SSPDLGLSPE | QIAQIRESYG |
| ADESLIAQYF | STLGGFLVGN | FGYSVQTGTA | VATQLAEALP | GTLTLAILAF | LLAAILALVI |
| SILATMDRFA | WIKGIFQALP | PFFVSLPSFW | LGIILIQIVS | FRLGWVPVIG | TTPAQGLILP |
| TITLSIPITA | PLAQVLIRSI | EEVKAQPFIA | AVRARGAGEM | WIFFRNIIRN | ALLPTLTIAG |
| ILFGELVGGA | VVTEAVFGRA | GLGQMTVNAV | ANRDMPVMLA | IVVIAA | |
| > RXA01012 (1-1641, translated) 547 residues | | | | | |
| MTTPLLEIND | LVVSYQTAKG | LVHAVNNVSL | EVHPGQITAI | VGESGSGKST | TAQAVIGLLA |
| DNAEVDSGRI | SFNGRSLVGL | NAREWKNVRG | TKIGLIPQDP | NNSLNPVKTI | GASVGEGLAI |
| HKRGTAAERK | KKVIELLERV | GIDNPEVRYD | QYPHELSGGM | KQRALIAAAI | ALEPELIIAD |
| EPTSALDVTV | QKIILDLLED | MQRELGMGIL | FITHDLAVAG | DRADRIVVMQ | KGEVRESGYA |
| ASVLTDPQHE | YSKKLLADAP | SLTIGEIPTR | VPAVDPEVAQ | AKGPLLVVDK | FRKEHQRGKE |
| GAFVAANDIS | FEVLPGTTHA | IVGESGSGKT | TLGRAIAMFN | TPTSGSISVS | GKDITNLSKA |
| QQRELRQQIQ | LVYQNPYSSL | DPRQTIGSTI | AEPLRNFTKV | SKQEADEKVA | HYLELVALDP |
| ALATRRPREL | SGGQRQRVAI | ARAMILEPEL | VVFDEAVSAL | DVTVQAQILR | LLDDLQRELG |
| LTYVFISHDL | AVVREISDTV | SVMSRGNQVE | LGKTAEVFNN | PQTDFTRRLI | DAIPGSRYRG |
| GELNLGL | | | | | |
| > RXA01013 (1-795, translated) 265 residues | | | | | |
| LGNPWTRPAA | VISIVVLAVA | VLMALVPGLF | TSQDPFTGDD | VALLGPSGTH | WFGTDSVGRD |
| LYSRVVYGAR | ETLLGALIAV | LVGLIVGTLI | GLLAGAQRGW | VDTVLMRFVD | VLLSIPALLL |
| SLTVIILLGF | GTMNAAIAVG | ITSVATFARL | ARSQVMTVAG | SDFVEAAYGS | GGTQAQVLFR |
| HILPNSLTPV | FALAALQFGS | AILQLSVLGF | LGYGAPAPTP | EWGLLISDAR | DYMATSWWLT |
| VLPGFVIIAV | VMSANYLSRI | IQKEA | | | |
| > RXA01070 (1-1386, translated) 462 residues | | | | | |
| MANATAQKGR | FGLPGWMTGF | GAQVIAGLIL | GLILGLVARG | MDSGAADGEA | SWLTGLLSGV |
| GSAYVSLLKV | MVPPLVFAAV | VTSVAKLREV | ANAARLAVST | LVWFAITAFF | SVLAGIAVAL |
| IMQPGVGSTV | DASNAADPSR | VGSWLGFIQS | VIPSNILGLS | GSYSENSGVN | LSFNVLQILV |
| ISIAIGVAAL | KAGKSAEPFL | KFTESFLKII | QIVLWWIIRL | APIGSAALIG | NAVATYGWSA |
| LGSLGKFVLA | IYVGLAIVMF | VIYPVVLKLN | GIPVLGFFKR | VWPVTSLGFV | TRSSMGVMPV |
| TQRVTEQSLG | VPSAYASFAI | PLGATSKMDG | CAAVYPAVAA | IFVAQFYGID | LSIMDYVLIM |
| IVSVLGSAAT | AGTTGATVML | TLTLSTLGLP | LAGVGLLLAI | EPIIDMGRTA | TNVTGQALVP |
| AIVAKREGIL | DQDVWDAAEK | GGAAIEMATV | SEKETEPAEV | RS | |
| > RXA01094 (1-948, translated) 316 residues | | | | | |
| MTLATIPSPP | QGVWYLGPIP | IRAYAMCIIA | GIIVAIWLTR | KRYAARGGNP | EIVLDAAIVA |
| VPAGIIGGRI | YHVITDNQKY | FCDTCNPVDA | FKITNGGLGI | WGAVILGGLA | VAVFFRYKKL |
| PLAPFADAVA | PAVILAQGIG | RLGNWFNQEL | YGAETTVPWA | LEIYYRVDEN | GKFAPVTGTS |
| TGEVMATVHP | TFLYELLWNL | LIFALLMWAD | KRFKLEHGRV | FALYVAGYTL | GRFWIEQMRV |
| DEATLIGGIR | INTIVSAVVF | AGAIIVFFLL | KKGRETPEEV | DPTFAASVAA | DAVASPDRKP |
| LPKAGEGIDG | ETPSTR | | | | |
| > RXA01135 (1-324, translated) 108 residues | | | | | |
| VTHILEDSRR | FLQLGAFASL | STALAGAARY | VTSTSNNEPA | DNTPLTIGYV | PIAGSAPIAI |
| ADALGLFKKH | GVNVTLKKYS | GWSDLWTAYA | TEQLDVAHML | SPMTVAIN | |
| > RXA01141 (1-462, translated) 154 residues | | | | | |
| VNSAADLKGM | VLGIPFEYSV | HALLLRDYLV | SNAVDPIADL | ELRLLRPADM | VAQLTVEGID |
| GFIGPGPFNE | RAISNGSGRI | WLLTKQLWDK | HPCCAVAMAK | EWKAEHPTAA | QGVLNALEEA |
| SAILSNPAQF | DSSARTLSQE | KYLNQPATLL | DGPS | | |
| > RXA01142 (1-420, translated) 140 residues | | | | | |
| TRTHLEQVGL | TDAAERRPAR | LSGGMQQRVG | IARAFAIDPP | IMLLDEPFGA | LDALTRRELQ |
| LQLLNIWEAS | RRTVVMVTHD | VDEAILLSDR | VLVMSKSPEA | TIITDIPVNL | PRPRHELSED |
| ASVEAETTAL | RKRMLHLLEH | | | | |
| > RXA01164 (1-1575, translated) 525 residues | | | | | |
| VTLFVRLALA | AVGGLFVFAS | NEPIGWFVAG | IVGTALFFIS | LAPWDLGVPQ | KRRKKNEPVP |
| FLQQMSTGPT | VVQGMLLGFV | HGLVTYLQLL | PWIGEFVGSL | PYVALSVVEA | LYSIALGAFG |
| VLIARWRDWK | VLLFPAMYVA | VEYLRSSWPF | DGFAWVRLAW | GQINGPLANL | AALGGVAFVT |
| FSTVLAAVGV | AMVIISKKRL | AGAIITASVI | AIGAVSSLYV | DRNGTSDESI | EVAAIQGNVP |
| RMGLDFNAQR | RAVLANHARE | TLKLDEQVDL | VIWPENSSDV | NPFSDAQARA | IIDGAVEHVQ |
| APILVGTITV | DEVGPRNTMQ | VFDPVEGAAE | YHNKKFLQPF | GEYMPFREFL | RIFSPYVDSA |
| GNFQPGDGTG | VVEMNAANLG | RAVTVGVMTC | YEVIFDRAGR | DAIANGAEFL | TTPTNNATFG |
| FTDMTYQQLA | MSRMRAIEFD | RAVVVAATSG | VSAIVNPDGS | ISQNTRIFEA | ATLTESIPLK |
| DTVTIAARVG | FYVELLLVII | GVLAGLFAIR | MNSRSKSAKG | SARPA | |
| > RXA01168 (1-720, translated) 240 residues | | | | | |
| RTATPDVHVL | IVDDNSPDGT | GERADKLAAD | DDHIFVLHRE | GKGGLCAEYM | AGFQWGLERD |
| YQVLCEMDAD | GSHAPEQLHL | LLAEITNGAD | LVIGSRYVPG | GRVVNWPKNR | WLLSKGGNVY |
| ISVALGAGLT | DMTAGYRAFR | REVLEALPLD | ELSNAGYIFQ | VEIAYRAVEA | GFDVREVPIT |
| FTEREIGESK | LDGSFVKDSL | LEVTKWGLKH | RGGQAKELSK | EMVGLLNYEW | KHFKKRNTWL |
| > RXA01185 (1-858, translated) 286 residues | | | | | |
| MTDPENSQGT | PQICPTDPTT | QALAVRGLTK | SYGDATVVNN | INLDIPKGAI | YGIVGPNGAG |
| KTTMLSMATG | LLRPNKGTAW | ISGFNVWEEP | NDAKRSMGLL | ADGLPIFDRL | TGKELLTYVG |
| ALRELDEGIV | DQRSEELLEA | LGLKEAAGKR | VVDYSAGMTK | KILLAQALIH | NPKVLILDEP |
| LEAVDPVSGR | LIQQILKNFA | QTGGTVVLSS | HVMELVEGLC | DHVAIINRGV | VEIAGHVNEV |
| RRGRSYRMSS | LMRLKALLFK | RGHYLGWVRP | KAIAKAKIRT | RIGLSK | |
| > RXA01188 (1-1104, translated) 368 residues | | | | | |
| MMNGVVQPQE | HLDATLIAAD | FHGNPENSGD | RKERLNFQGW | KYALNRTVRD | VFPDGLLDLA |
| ALLTFFSILS | IAPAVLLGYS | VITIFLASDS | TEILNLVRDE | VNQYVPEDQS | HVVNGVIDSI |
| AGSAAAGQVG | VAVGVITALW | TSSAYVRAFS | RCANAVYGRS | EGRTLIKRWA | MLLFLNLALL |
| LGIIIILVSW | VLNETLVMGI | FAPIAEPLHL | TNVLSFLTDR | FMPIWIWVRF | PVIVGVLIMF |
| VATLYYWAPN | ARPWKFRWLS | LGSFLAIVGI | LLAGVGLNFY | FTLFAAFSSY | GAVGSLLAVF |
| IALWVFNICL | IIGLKIDVEI | SRAKQLQAGM | PAEDYSLVPP | RSIEKVAKMK | QRQQRLMDQA |
| AAIREESN | | | | | |
| > RXA01245 (1-1767, translated) 589 residues | | | | | |
| ASWVTTLGLG | GEHLDFWWEL | ALLVTIMLLG | HWLEMRALGA | ASSALDALAA | LLPDEAEKVV |
| DGTTRTVAIS | ELAVDDVVLV | RAGARVPADG | TIMDGAAEFD | EAMITGESRP | VYRDTGETVV |
| AGTVATDNTV | RIRVEATGGD | TALAGIQRMV | ADAQASSSRA | QALADRAAAL | LFWFALITAL |
| ITAVVWTIIG | SPDDAVVRAV | TVLIIACPHA | LGLAIPLVIA | ISSERAAKSG | VLIKDRMALE |
| HMRTIDVVLF | DKTGTLTEGA | HAVTGVAPAT | GIAEGELLAL | AAAAEADSEH | PVARAIVTAA |
| AAHPEASQRQ | LRATGFTAAS | GRGIRATVDG | AEILVGGPNM | LREFNLTTPG | ELADITGSWA |
| QRGAGVLHVV | RDGEIIGAVA | VEDKIRPESR | AAVRALQARG | VKVAMITGDA | TQVAQAVGKD |
| LGIDEVFAEV | LPQDKDTKVT | QLQERGLSVA | MVGDGVNDAP | ALARAEVGIA | IGAGTDVAME |
| SAGVVLASDD | PRAVLSMIEL | SHASYRKMVQ | NLVWATGYNI | VAVPLAAGVL | APIGVLLPPA |
| AAAILMSLST | IIVALNAQLL | RRIDLDPAHL | APTDGKEEKA | AVSSAAPVR | |
| > RXA01247 (1-234, translated) 78 residues | | | | | |
| VAAATDATPE | GPTTYQVTGM | TCGHCADNVT | EAVSALPQVD | DVQVDLIAGG | VSIVTVTGSV |
| PLETVHRAIE | ETGYTVLS | | | | |
| > RXA01285 (1-543, translated) 181 residues | | | | | |
| PQTSIAPEGI | RVYDLIARGR | APYQSLIQQW | RTSDEDAVAQ | ALASTNLTEL | AARLVDELSG |
| GQRQRVWVAM | LLAQQTPIML | LDEPTTFLDI | AHQYELLELL | RAFNEAGKTV | VTVLHDLNQA |
| ARYADHLIVM | KDGHVHATGT | PEEVLTAEMV | QGVFGLPCII | SPDPVTGTPT | VVPLSRSRAG |
| A | | | | | |
| > RXA01289 (1-1044, translated) 348 residues | | | | | |
| MTAVAVEKQK | ETSISKNLGR | RRALGILGIV | VALGALIVLS | IAVGANPLSF | SSVWQGFTAH |
| DSSEASIIVW | SMRIPRTLVG | IVTGAAFGVA | GALIQALTRN | PLADPGILGV | NAGAGFAVTV |
| GVGFFGLSSV | TGYIWFAFLG | AAAATLLVYF | IGASTSGSVN | PVALVLAGVA | LAAVLGGVTS |
| FLTLIDPETF | ESIRNWNLGS | VARTDLSDTM | TVLPFLAVGL | AIALLLSGAL | NSIALGDDLA |
| ASLGTKVMRT | RVLGIISVTL | LAGGATALTG | GIGFVGLMVP | HVVRWVVGPD | QRWIITFSAL |
| CAPVLVLGAD | ILGRIIARPG | EIEVGIVTAV | IGAPVLIALV | RRRKASGL | |
| > RXA01290 (1-1164, translated) 388 residues | | | | | |
| VVFNIKSRTD | ETPVAASEPV | ESTRPVSEAS | TSPALNPGYH | AVSVQRRRFS | FRIPARLMVV |
| SLILFAIALC | SATWAITMGD | YPLSLGQVIN | ALAGTGEKFQ | LLVVREWRLP | VAIAAVVFGA |
| LLGTGGAIFQ | SITRNPLGSP | DVIGFDAGSY | TAVVLVILVL | GNTHYWSIAF | AAIVGGIVTA |
| FAVYVLAWRK | GVQGFRLIIV | GIGVSAMLSS | VNAYLITRAD | VEDAMVVGFW | SAGSINRITW |
| QSLLPSLVIA | AVIIVAAIVL | ARSLRFMEMG | DDVATTLGVK | TNSTRLALIV | VGVATSALVT |
| AAAGPISFIA | LVAPQLARRL | TKTPGVSLVA | AAAMGSALLS | CAHLLSLIIS | SFYRTIPVGL |
| LTVSIGGCYM | IWLLLRETRR | QYRTGTIR | | | |
| > RXA01297 (1-798, translated) 266 residues | | | | | |
| MGYVGMVLAI | LEIGLPLVFI | VLTSFKQQSE | IYTQPVTWFP | SEFNFDNYAN | VFERVPFLNY |
| FRNSIIITVI | LCLVKIILGV | ISAYALSILR | FPGRNLVFLL | VISALMVPSE | VTVISNYALV |
| SQLGWRDTYQ | GIIVPLAGIA | FGTFLMRNHF | MSIPSELIEA | ARMDHCGHFR | LLWKVLLPIS |
| MPTLVAFSMI | TVVNEWNQYL | WPFLMAETDN | SATLPIGLTM | LQNNEGVSNW | GPVMAATIMT |
| MLPVLVMFLA | LQEYMIKGLI | SGAVKG | | | |
| > RXA01298 (1-393, translated) 131 residues | | | | | |
| FVWKNLGYSF | VIYLAALQGL | NKDLSEAAPV | DGASAWTRFW | KVTLPQLRPT | TFFLSITVTL |
| NSVQVFDIIH | TMTRGGPLGN | GTTTLVYQVY | TETFTNYRAG | YGATIATILF | LLLLIITVIQ |
| VRYMDKENKQ | K | | | | |
| > RXA01303 (1-1335, translated) 445 residues | | | | | |
| VTQLNTKGVV | LQGWDPEDPE | HWDSKIAWRT | LWITTFSMII | GECVWYLVSA | IAPLLNRIGF |
| DLSAGQLYWL | ASIPGLAGGL | IRLIYMELPP | ILGTRKLVGI | SSGLFLIPMF | GWFLAVQDSS |
| TPYWWLLTLA | ALTGIGGGVF | SGYMPSTGYF | FPKAKSGTAL | GIQAGIGNLG | VSIIQFMGPW |
| VMGFGLLGIG | FLTPQRTIEG | TTVFVHNAAI | VLVPWTILAA | VLSFLFLKDV | PVTANFRQQI |
| DIFGNKNTWI | LSIIYLMTFG | AFAGFAAQFG | LIINNNFGIA | SPMAETYPAE | MLHAGATFAF |
| LGPLIGALVR | AAWGPLCDRF | GGAIWTFVGG | IGMTIATAAA | AIELSRAETP | DDEWPFLWSM |
| LALFFFTGLG | NAGTFKQMPM | ILPKRQAGGV | IGWTGAIGAF | GPEIVGVLLS | FTPTVAFFWG |
| CVVFFIIATA | LTWIYYARPN | APFPG | | | |
| > RXA01323 (1-2265, translated) 755 residues | | | | | |
| MAQTPAKIPA | ALNFIDVDLG | VTGMTCTSCS | ARVERKLNKL | DGVEATVNYA | TESAQVSYDP |
| SKVSPEQLIK | TVEDTGYGAF | TMASAAAESE | EDNAPADSGQ | SRIDAARDHE | AADLKHRVIV |
| SALLSVPVVL | VSMIPALQFN | NWQWAVLTLV | TPIFFWGGSP | FHKATWANLK | RGSFTMNTLV |
| SLGTSAADLW | SLWALFIENA | GHPGMKMEMH | LLPSASTMDE | IYLETVAVVI | TFLLLGRWFE |
| TKAKGQSSEA | LRKLLDMGAK | DAVVLRDGAE | VRVPVNQLKL | GDVFITRPGE | KIATDGEVDE |
| GSSAVDESML | TGESIPVEVT | KGSKVTGATL | NTSGRLMVKV | TRIGADTTLS | QMAKLVTDAQ |
| SKKAPVQRLV | DQISQVFVPV | VIVIATATLI | AHLVFTDAGL | APAFTAAVAV | LIIACPCALG |
| LATPTALLVG | TGRGAQLGLL | IKGPEILEST | KKVDTIVLDK | TGTVTTGTMS | VTDVTAINYS |
| ETEILEFAAA | VESASERPIA | QAIAKAAEHE | QVTDFQNTAG | QEVTGVVRGH | EVRVGRPSST |
| LIDALLHPFQ | HAQKIGGTPV | VVTIDGVDSG | IITVRDTVKD | TSAEAIRGLK | ELGLTPILLT |
| GDNEGAAKSV | AAEVGIDQVI | ANVLPHEKVQ | NVEALQAQGK | NVAMVGDGVN | DAAALAQADL |
| GLAMGAGTDV | AIEASDITLM | NNDLRSAVDA | IRLSRKTLGT | IKGNLFWAFA | YNVALIPVAA |
| IGLLNPMLAG | IAMAFSSVFV | VSNSLRLRGF | KARSN | | |
| > RXA01338 (1-1878, translated) 626 residues | | | | | |
| MLFIRSFDGI | ITVAALVAIA | IHLILWLALD | LDGLAKNWPL | IAIVIVGGIP | LMWDVLKSAI |
| KTRGGADTLA | AVSIITSVLL | GEWLVAAIIV | LMLSGGEALE | EAASRRASGT | LDALARRAPS |
| TAHRLLGATI | LDGTEEIAVE | EITVGDLVAV | LPHELCPVDG | EIVAGHGTMD | ESYLTGEPYV |
| VSKSKGSQAM | SGAVNGDTPL | TIVATKLAHD | SRYAQIVGVL | HEAENNRPEM | RRMADRLGAW |
| YTVIALALGG | LGWIVSGDPV | RFLAVVVVAT | PCPLLIAVPV | AIIGAISLAA | RRGIIVKNPG |
| MLENASGVKT | VMFDKTGTLT | YGRPVITDIH | TAPGVEEDTV | LALAASVERY | SRHPLADAIR |
| EGAKARELHL | PDVVEVSERP | GQGLTGTVGE | HLVRITNRRS | TLEIDPDSKN | YIPVTSSGME |
| SVVLVDDKYA | ALIRLRDEPR | ASASEFIAHL | PKKHKVDKLM | IISGDRASEV | RYLADKVGID |
| EVHAEASPED | KLNIVNRHNE | HGATMFLGDG | INDAPAMAVA | TVGVAMGADS | DVTSEAADAV |
| ILDSSLERLD | DLLHISARMR | RIALQSAGGG | MALSVIGMIL | AVFGFLTPLM | GAIFQEVIDV |
| LAILNSARVA | LPRGAISDFD | TQEKVS | | | |
| > RXA01395 (1-1086, translated) 362 residues | | | | | |
| MAVMAYQPAD | NRYDDMIYRR | VGNSGLKLPA | ISLGLWHNFG | DDKPLSTQRS | IIHRAFDRGV |
| THFDLANNYG | PPAGSAETNF | GRILREDLKS | HRDELIISSK | AGWDMWPGPY | GFGGSRKYLV |
| SSLDQSLTRL | GLDYVDIFYH | HRPDPDTPLE | ETMYALRDIV | ASGKALYVGI | SSYGPELTAE |
| AAEFMAEEGC | PLLIHQPSYS | IINRWVEEPG | DDCENLLQSA | ANNGLGVIAF | SPLAQGLLTD |
| KYLDGIPEGS | RASQGKSXXX | XXLNVNNIDX | VXXXXXXSXX | TGQSFXXKXF | CWVVAQPRKV |
| RRRITVTSAL | IGASSVEQLD | NSLDSLNNLE | FSDAELEAID | EISHDAGINI | WAKATDSKTR |
| EN | | | | | |
| > RXA01411 (1-327, translated) 109 residues | | | | | |
| FIAQVMLGIG | AVTANCVTSV | MMAEVPQEVT | RGTSAGITYN | VTYAIFGGSA | PFISTALVSW |
| TGSPLAPAVY | MIIIALFAFT | ASRFIPETSP | VFVTATPAIK | APKVLVNPG | |
| > RXA01454 (1-267, translated) 89 residues | | | | | |
| MMLIVAFLIA | LVGHYLMGGI | RAGNQMTGQK | SFVSRGARTQ | LAVTAGLWML | VKVAGYWLDR |
| YDLLTKENST | FTGASYTDIN | AQLPAKIIL | | | |
| > RXA01455 (1-462, translated) 154 residues | | | | | |
| VTWIFAIIAL | VILIAPMSVG | FYTDWLWFGE | VDFRGVFSKV | IVTRIVLFVI | FALIAGEVTW |
| LAGYFVTKLR | PDEMSAFDTQ | SPVYQYRQMI | ENSLRRVMVI | IPIEVALLAG | LIGQRSWRTV |
| QMWLNGQDFG | VSDQQFGLDY | GFYAFDLPML | RLIA | | |
| > RXA01625 (1-201, translated) 67 residues | | | | | |
| MAIKNYTVEG | MTCGHCVSSV | KEEVGEVAGV | TAVDVTLETG | AVQVTGEDFT | DEAVKAAVVE |
| AGYKVVA | | | | | |
| > RXA01756 (1-1308, translated) 436 residues | | | | | |
| MKELELGEAR | DVAATLEAMP | IQEVIDQVER | TSITKGAVLL | RLLSKDRSLL | VFDALGPRLQ |
| ADLIGAFQDA | EVLDYEADLD | PDDRVSLLDE | LPASIADELL | RSLDPQEKQV | TELVLGYAKG |
| SVGRWMSPQV | LLLFDDMSVA | EVLDEVRNHA | AEAETIYALP | IVNRARQVMG | VVSLRKLFIA |
| DPTLKVSEIM | VRPVSVLASA | DIEETARWFL | QLDLVAMPVV | DESNMLLGVL | TFDDAQDIVE |
| QADSEDSARS | GGSEPLQQPY | LSTPIRKLVK | SRIVWLLVLA | VSAILTVQVL | DIFEATLVEA |
| VVLALFIPLL | TGTGGNTGNQ | AATTVTRALA | LGDVRKSDVF | RVLGREIRVG | LMLGALLGAV |
| GEVIASLVYG | MPVGTVIGLT | LLAVCTMAAS | VGGVMPIIAK | AIGADPAVFS | NPFISTFCDA |
| TGLIIYFAIA | KLVLGI | | | | |
| > RXA01808 (1-1119, translated) 373 residues | | | | | |
| MRGGAPARTS | KPGFRLEAAE | ALIAEVPAPR | DKVELMAFSK | SRQGRVVIEL | EDATVATPDD |
| RILVEDLTWR | LAPGERIGLV | GVNGSGKTTL | LRTLAGEQPL | QAGKRIEGQT | VKLGWLRQEL |
| DDLDLSRRLI | DCVEDVASYV | MMGDKQVSAS | QLAERLGFSP | KRQRTPVGDL | SGGERRRLQL |
| TRVLMAEPNV | LLLDEPTNDL | DIDTLQELES | LLDGWPGTMV | VISHDRYLIE | RVTDSTWALF |
| GDGKLTNLPG | GIEEYLQRRA | AMAAAEDSGV | LNLGAATQAG | TFSAATEQAA | TSVESSGISS |
| QERHRITKEM | NALERKMGKL | DQQMDKLNQQ | LADAAEAMDT | IKLTELDTKL | RAVQEEHGEL |
| EMQWLELGEE | IEG | | | | |
| > RXA01822 (1-582, translated) 194 residues | | | | | |
| MARQNSNTGG | LRLVLVGIGT | GAFLGAARDE | FMVRADITGA | STVQLWSAGS | LSGRDWNHAL |
| LVLISCAVIV | PALCIIVRRL | RLMEMGDDAA | GALGISVERT | RLIAILLAVL | LVGIATAAAG |
| PIAFIALAAP | QIARALARED | GVLVAASISI | GSGLLVAADC | LEQHVDTELH | TPVGLVTSLL |
| GGVYLMWLLS | RKEA | | | | |
| > RXA01890 (1-720, translated) 240 residues | | | | | |
| MASIVFENVT | RKYSPGARPA | VDKLNLEIAD | GEFLVLVGPS | GCGKSTSLRM | LAGLEPIDEG |
| RLLIDGKDAT | ELRPQDRDIA | MVFQSYALYP | NMTVRDNMGF | ALKNQKVAKA | EIEKRVAEAS |
| RILQLDPYLD | RKPAALSGGQ | RQRVAMGRAI | VREPSVFCMD | EPLSNLDAKL | RVSTRAEISG |
| LQRRMGVTTV | YVTHDQVEAM | TMGDRVAVLL | LGVLQQVDTP | QNLYDYPANA | FVASPIGSLP |
| > RXA01900 (1-1299, translated) 433 residues | | | | | |
| MTTAVDQNSP | PKQQLNKRVL | LGSLSGSVIE | WEDFLVYGTV | AALVFNKMYF | PSGNEFLSTI |
| LAYASFSLTF | FFRPIGGVIF | AHIGDRIGRK | KTLFITLMLM | GGGTVAIGLL | PDYNAIGIWA |
| PILLMFLRIL | QGIGIGGEWG | GALLLAYEYA | PKKQRGLYGA | VPQMGISLGM | LLAAGVISLL |
| TLMPEDQFLT | WGWRIPFVGS | ILLVFIGLFI | RNGLDETPEF | KRIRDSGQQV | KMPLKEVLTK |
| YWPAVLVSIG | AKAAETGPFY | IEGTYIVAYA | TNFLNIRDNI | VLLAVACAAL | VATIWMPLFG |
| SFSDRVNRAV | LYRICASATI | VLIVPYYLVL | NTGEIWALFI | TTVIGFGILW | GSVNAILGTV |
| IAENFAPEVR | YTGATLGYQV | GAALFGGTAP | IIAAWLFEIS | GGQWWPIAVY | VAACCLLSVI |
| ASFFIQRVAH | QEN | | | | |
| > RXA01939 (1-603, translated) 201 residues | | | | | |
| STSGTDLTSL | SHKEIFQMRR | KLQVVEQNPY | GSLDPMYSIY | RCIEEPLTIH | KVGGDRKARE |
| ARVVELLDMV | SMPRSTMRRY | PNELSGGQRQ | RIATARALAL | NPEVIVLDEA | VSALDVLVQN |
| QILTLLAELQ | QELKLTYLFI | THDLAVVRQT | ADDVVVMQKG | RIVEKGRTDD | IFNDPQQHYT |
| RDLINAVPGL | GIELGTGENL | V | | | |
| > RXA01972 (1-594, translated) 198 residues | | | | | |
| VATGLLSAIG | LFIATNIDDI | IVLSLFFARG | AGQKGTTLRI | LAGQYLGFMG | ILAAAVLVTL |
| GAGAFLPAEA | IPYFGLIPLA | LGLWAAWQAW | RSDDDDDDDA | EIAGKKVGVL | TVAGVTFANG |
| GDNIGVYVPV | FLNVDTAAVI | IYCIVFLVLV | AGLVLLAKFV | ATRPPIAEVL | ERWEHVLFPI |
| VLIGLGIFIL | VSGGAFGL | | | | |
| > RXA01986 (1-618, translated) 206 residues | | | | | |
| MASTFIQADS | PEKSKKLPPL | TEGPYRKRLE | YVALVATFGG | LLFGYDTGVI | NGALNPMTRE |
| LGLTAFTEGV | VTSSLLFGAA | AGAMEFGRIS | DNWGRRKTII | SLAVAFFVGT | MICVFAPSFA |
| VMVVGRVLLG | LAVGGASTVV | PVYLAELAPE | EIRGSLAGRN | ELMIVVGQLA | AFVINAIIGN |
| VFGHHDGVWR | YMLAIAAIPA | IALFFG | | | |
| > RXA01995 (1-654, translated) 218 residues | | | | | |
| MDIRQTINDT | AMSRYQWFIV | FIAVLLNALD | GFDVLAMSFT | ANAVTEEFGL | SGSQLGVLLS |
| SALFGMTAGS | LLFGPIGDRF | GRKNALMIAL | LFNVVGLVLS | ATAQSAGQLG | VWRLITGIGI |
| GGILACITVV | ISEFSNNKNR | GMAMSIYAAG | YGIGASLGGF | GAAQLIPTFG | WRSVFAAGAI |
| ATGIATIATF | FFLPESVDWL | STRRPAGARD | KINYIARR | | |
| > RXA02033 (1-789, translated) 263 residues | | | | | |
| MPLSGKIGGF | IVAVVFVLAA | LSFIWTPFDP | VQAFPQERLE | GSSLRHLLGT | DRYGRDVLSQ |
| IMVGSRVTLL | VGIIAVAIAA | LIGTPLGIAA | GMRRGMVETF | VMRGADLMLA | FPALLLAIIS |
| GAVFGASTWS | AMVAIGIAGI | PSFARVARAG | TLQVTSQDFI | AAARLSKVSS | ARIALRHILP |
| NITSMLIVQA | SVAFALAILA | EAALSFLGLG | TTPPDPSWGR | MLQTAQASIG | VTPMLAVWPG |
| AAIALTVLGF | NLFGDGLRDA | IDP | | | |
| > RXA02034 (1-966, translated) 322 residues | | | | | |
| VSKTIAWTVL | RYTLTFVIAS | IIIFVLIRVI | PGDPAAVALG | ITATPEAIAA | LQSQLGTDQP |
| LFQQYFSWIG | GMLTGDFGTS | LSSGQDLSPI | IFDRLQVSLI | LVGCSIVLSL | LIAIPLGVLS |
| ARRGGVIISG | ISQIGIAIPS | FLAGILLVAV | FAVGLGWLPA | NGWIPPSENF | GGFLARLILP |
| VLALTAVQAA | ILTRYVRSAV | MDVMGQDFMR | TARSKGMSFN | RALIIHGLRN | AALPVLTVTG |
| LQLTTLVIGA | VVIEQVFVIP | GIGSMLLESV | SNRDLIAVQS | IVMLLVAFTL | LVNLVVDLLY |
| QVVDPRVGAV | GVASTKVPGS | VA | | | |
| > RXA02035 (1-1509, translated) 503 residues | | | | | |
| MKITRGLLPS | LLLASTIVVS | SCSAGSTAYQ | QPPAVDQSSI | VIATTAAAAS | LDFTNAAGAA |
| IPQAMMSNIY | EGLVRIDAEG | EIQPLLATSW | DISDDRTEYI | FHLREGVLFS | NGDPFNADSA |
| KFSIDRVKTD | WTNGLKSGMD | VVESTEVIDD | HTLKVSLVRP | SNQWLWSMGT | AIGAMMTEGG |
| VDKLATDPVG | TGPYTVTHWA | PGRAIGFGAR | ADYWGQKPLN | DAATIRYFSD | ATASTNALQS |
| GDVDVIWAMQ | APEQLATLQE | YTVEVGTTNG | EMLLSMNNQR | APFDDVRVRQ | AVMFAIDRQA |
| VIDTALEGYG | TDTGGVPVPP | TDPWYEKSTM | YPYDPDRARA | LLEEAGAEGT | RITMSIPSLP |
| YAQAASEILY | SQLRDVGFDP | VIESTEFPAV | WLAQVMGQKD | YDMSLIAHVE | PRDIPTLFSP |
| NYYLGFDDTE | TQALLAEADS | SANEVELMQQ | AVDRIMEQAV | ADNLMNVANI | VVMSPEITGI |
| DPNVVSGALE | LSLIGRKESG | VAQ | | | |
| > RXA02062 (1-1170, translated) 390 residues | | | | | |
| MRVGMMTREY | PPEVYGGAGV | HVTELTRFMR | EIAEVDVHCM | GAPRDMEGVF | VHGVDPALES |
| ANPAIKTLST | GLRMAEAANN | VDVVHSHTWY | AGLGGHLAAR | LHGIPHVATA | HSLEPDRPWK |
| REQLGGGYDV | SSWSEKNAME | YADAVIAVSA | RMKDSILAAY | PRIEPDNVRV | VLNGIDTELW |
| QPRPTFDDAE | DSVLRSLGVD | PQRPIVAFVG | RITRQKGVEH | LIKAAALFDE | SVQLVLCAGA |
| PDTPEIAART | TALVEELQAK | REGIFWVQDM | LGKDKIQEIL | TAADTFVCPS | IYEPLGIVNL |
| EAMACNTAVV | ASDVGGIPEV | VVDGTTGALV | HYDENDVETF | ERDIAEAVNK | MVADRETAAK |
| FGLAGRERAI | NDFSWATIAQ | QTIDVYKSLM | | | |
| > RXA02068 (1-1119, translated) 373 residues | | | | | |
| IFVPMLRIAA | IEPKDITLVT | GSVSLRTFRV | RTGELQVMGD | IVGAKVHTDD | PELQQFHGRA |
| VETADVELEL | SRTRDWIITR | VAVLGERPKF | GRRPVLHTVP | WSHIHGITAG | GVGESNHTAE |
| LIAGFEDMRP | ADVAKQLYQL | PTAQRTEVTE | ELDDEKLADI | LQELSEDRQA | ELIEELDIER |
| AADILEEMDP | DDAADLLGEL | PDDKADVLLD | LMDPEESAPV | RRLMDFSPDT | VGALMTPEPL |
| IMDPSTTVAE | ALAMARNPDL | PTSLASLIFV | VRPPTATPTG | KYLGCVHLQK | LLREPPSSLI |
| GGILDPDLPP | LYADDEQETA | ARFFATYNLV | CGPVLDENRH | LLGAVAVDDL | LDHMLPEDWR |
| DAGIRPGKEH | THG | | | | |
| > RXA02079 (1-615, translated) 205 residues | | | | | |
| MSEAFDATKV | RKAVLTVALL | NFAYFFVEFF | IALSAGSVSL | LADSVDFLED | TSINLLIFIA |
| LGWPLARRAV | MGKLMAIVIL | APAAFAAWAA | IQRFSAPQAP | EVFPIIVASL | GAVVINGASA |
| IIISRVRQHG | GSLGQAAFLS | ARNDVLINIA | IIMMALITAW | TTSGWPDLIL | GCFIILLALH |
| AAHEVWEVSE | EERLASKALA | GEAID | | | |
| > RXA02096 (1-1317, translated) 439 residues | | | | | |
| MGLDVSDEQI | EHAARLAQAH | DFIDRLPNKY | EEVIGERGLT | LSGGQRQRIA | LARAFLARPK |
| VLVLDDATSA | IDASTEDRIF | QALREELHDV | TILIIAHRHS | TLELGDRVGL | VEDGRVTALG |
| PLSEMRDHAR | FSHLMALDFQ | DSHDPEFTLD | NGSLPSQEQL | WPEVSTEKQY | KILAPAPGRG |
| RGMSMPATPE | LLAQIEALPA | ATEETRVDAG | RLRTSTSGFK | LLSLFKQVRW | LVVAVIALLL |
| VGVAADLAFP | TLMRAAIDNG | VQAQSTSTLW | WIAIAGSVVV | LLSWAAAAIN | TIITARTGER |
| LLYGLRLRSF | VHLLRLSMSY | FERTMSGRIM | TRMTTDIDNL | SSFLQSGLAQ | TVVSVGTLIG |
| VVTMLAITDA | QLALVALSVV | PIIIVLTLIF | RRISSRLYTA | SREQASQVNA | VFHESIAGLR |
| TAQMHRMEDQ | VEDNYAGEA | | | | |
| > RXA02119 (1-1641, translated) 547 residues | | | | | |
| MTETLVVNGL | AGGYGHRTLF | NDVNLTVAAG | DVVGVVGVNG | AGKSTELKIL | AGVEKPLAGT |
| IALSPADAFV | GYLPQEHTRT | SGETIAVYIA | RRTGCQAATT | AMDDTAEAFG | ADPDNAALAD |
| AYAEALDRWM | ASGAADLDER | IPIVLADLGF | ELPTSTLMEG | LSGGQAARVG | LAALLLSRFD |
| IVLLDEPTND | LDLDGLEQLE | NEVQGLRGGV | VFVSHDREFL | SRCVTTVLEL | DLHQNSHHVY |
| GGGYDSYLEE | RAVLRQHARD | QYEEFAEKKK | DLVARARTQR | EWSSHGVRNA | IKRAPDNDKL |
| RKKAAAESSE | KQAQKVRQME | SRIARLEEVE | EPRKEWKLQF | SVGKASRSSS | VVSTLNDASF |
| TQGDFTLGPV | SIQVNAGDRI | GITGPNGAGK | STLLRGLLGN | QEPTSGTATM | GTSVAIGEID |
| QARALLDPQL | PLISAFEKHV | PDLPISEVRT | LLAKFGLNDN | HVERDVEKLS | PGERTRAGLA |
| LLQVRGVNVL | VLDEPTNHLD | LEAIEQLEQA | LASYDGVLLL | VTHDRRMLDA | VQTNRRWHVE |
| AGEVREL | | | | | |
| > RXA02220 (1-2676, translated) 892 residues | | | | | |
| VSSPLPAAVT | SKPAHALSSD | EVLENLGVQD | TGLTSAEATQ | RLEANGPNEL | PQTPPETVWQ |
| RLFRQVNDPM | IYVLIAAAVL | TAELGHWTDT | IVIGAVVIIN | MMVGFIQEGK | AADALASIRN |
| MLSPESAALR | DGVFHKIDAA | ELVVGDVVKL | SAGDKVPADL | RMLAATNLHI | EESALTGEAE |
| AVVKGTDPVE | ADAGIGDRTS | MAFSGTLVLT | GSGTGVVTAT | GAGTEIGHIT | TMLADVDSVD |
| TPLTRSMKKF | SSALAIVCVF | LAILMLVVAG | LVHHTPLEEL | ILSAIGFAVA | AIPEGLPAVI |
| AITLALGVQK | MAARNAITRR | LNSVETLGSV | TTICTDKTGT | LTRNEMTVRA | IATGTSLYDV |
| SGAGYEPLGE | IRLKDGEQVS | KQDFPDLYAM | ALVAANVNDA | EIYQEDGMWR | LSGEPTDGGI |
| RAFAMKTNAE | ILTRTAEVPF | DSAYKYMATL | HTIDGANTML | VKGAPDRLLD | RSAQQRNGEP |
| LDRPYWEQLI | EDLASQGLRV | LAAAYKELPH | STSTITPEDV | DQGELTFLGL | YGIMDPPREE |
| VIEAMKVVQS | AGVRVRMITG | DHSSTARAIA | REVGIRGQNV | LTGAEITAAT | DEELQGLVDN |
| ADLFVRTSPE | HKLRVVRALQ | ANGEVASMTG | DGVNDAPALK | QADVGVAMGI | KGTEATKDAA |
| DIVLADDNFA | TIAGAVEMGR | TIYDNLRKAV | VFMLPTNGAQ | GLVIFIAMLL | GWELPITALQ |
| VLWINLITAI | TLSLALSFEP | AEPGIMNRKP | RNPKSGLIDA | PSVLRIVYVS | LLLGGATFWA |
| FLGARDAGID | IDTARTIAVT | TLAVSQVFYL | LSSRYFEVSA | LRKELFTTNP | ISWLCIALML |
| ILQLAFVYLP | FMQSTFDTAA | LTLRDWVMPL | VFGVVVFAVV | ETEKFIRRLK | AS |
| > RXA02222 (1-375, translated) 125 residues | | | | | |
| LGRPPPGDVH | TLLDDIGAEE | SEADKVPIEW | QNALTKADRY | ANRQHMSQAR | LYRQLTSDVG |
| EGFTEEAAQY | AIENVNADWN | ANALVKARNY | QERQANSVDR | IYRQLTSEHG | EGFTPEQAQY |
| AIDNL | | | | | |
| > RXA02312 (1-1359, translated) 453 residues | | | | | |
| LSNRHLQLIA | IGGAIGTGLF | MGSGKTISVA | GPSVILVYAI | IGFMLFFVMR | AMGELLLANL |
| NYKSLRDAVS | DILGPGAGFV | TGWTYWFCWI | ATGMADIVAI | TGYTQYWWPE | IPLWLPGVLT |
| IALLFALNLA | AVRLFGEMEF | WFAIIKIVAI | VSLIVVGLFM | VVTAFESPNG | TTAQFNNLIE |
| HGGFFPNGIT | GFLAGFQIAI | FAFVGIELAG | TAAAETENPT | KTLPRAINSI | PIRIVVFYVL |
| ALAVIMMVTP | WDQVRADNSP | FVQMFALAGI | PAAAGIINFV | VITSAASSAN | SGIFSTSRML |
| YGLSLEGAAP | KRWSRLSKNL | VPARGLTFSV | ICLIPAVGLL | YAGGTVIEAF | TLITTVSSVL |
| FMVVWSYILV | AYIVYRRNSP | ELHKKSIFKM | PGGVVMAVVV | LVFFAAMLVV | LSLEPDTRAA |
| LIATPVWFII | LGIGWLSIGG | AKGAKHRSQI | TSH | | |
| > RXA02313 (1-1221, translated) 407 residues | | | | | |
| MRVAIVAESF | LPNVNGVTNS | VLRVLEHLKA | NGHDALVIAP | GARDFEEEIG | HYLGFEIVRV |
| PTVRVPLIDS | LPIGVPLPSV | TSVLREYNPD | IIHLASPFVL | GGAAAFAARQ | LRIPAIAIYQ |
| TDVAGFSQRY | HLAPLATASW | EWIKTVHNMC | QRTLAPSSMS | IDELRDHGIN | DIFHWARGVD |
| SKREHPGKRS | VALRKSWDPS | GAKKIVGFVG | RLASEKGVER | LAGLSGRSDI | QLVIVGDGPE |
| AKYLQEMMPD | AIFTGALGGE | ELATTYASLD | LFVHPGEFET | FCQAIQEAQA | SGVPTIGPRA |
| GGPIDLINEG | VNGLLLDVVD | FKETLPAAAE | WILDDSRHSE | MCAAAWEGVK | DKTWEALCTQ |
| LLQHYADVIA | LSQRIPLTFF | GPSAEVAKLP | LWVARALGVR | TRISIEA | |
| > RXA02344 (1-678, translated) 226 residues | | | | | |
| MLNRMKSARP | KSVAPKSGQA | LLTLGALGVV | EGDIGTSPLY | SLHTAFSMQH | NKVEVTQENV |
| YGIISMVLWT | ITLIVTVKYV | MLVTRADNQG | QGGILALVAL | LKNRGHWGKF | VAVAGMLGAA |
| LFYGDVVITP | AISVLSATEG | LTVISPSFER | FILPVSLAVL | IAIFAIQPLG | TEKVGKAEGP |
| IMLLWFVTLA | GLGIPQIIGH | PEILQSLSPH | WALRLIVAEP | FQAEVL | |
| > RXA02348 (1-1134, translated) 378 residues | | | | | |
| PIRVAWFCVV | MPALILTYLG | QGALVINQPE | AVRNPMFYLA | PEGLRIPLVI | LATIATVIAS |
| QAVISGAYSL | TKQAVNLKLL | PRMVIRHTSR | KEEGQIYMPL | VNGLLEVSVM | VVVLVFRSSE |
| SLASAYGLAV | TGTLVLVSVL | YLIYVHTTWW | KTALFIVLIG | IPEVLLFASN | TTKIHDGGWL |
| PLLIAAVLIV | VMRTWEWGSD | RVNQERAELE | LPMDKFLEKL | DQPHNIGLRK | VAEVAVEPHG |
| TSDTVPLSLV | RCVKDLKLLY | REIVIVRIVQ | EHVPHVPPEE | RAEMEVLHHA | PIRVVRVDLH |
| LGYFDEQNLP | EHLHAIDPTW | DNATYFLSAL | TLRSRLPGKI | AGWRDRLYLS | MERNQASRTE |
| SFKLQPSKTI | TVGTELHL | | | | |
| > RXA02353 (1-468, translated) 156 residues | | | | | |
| MALLILAGLQ | MIPKETYEAA | RVDGATAWQQ | FTKITLPLVR | PALMVAVLFR | TLDALRMYDL |
| PVIMISSSSN | SPTAVISQLV | VEDMRQNNFN | SASALSTLIF | LLIFFVAFIM | IRFLGADVSG |
| QRGIKKKKLG | GTKDEKPTAK | DAVVKADSAV | KEAAKP | | |
| > RXA02354 (1-789, translated) 263 residues | | | | | |
| MTKRTKGLIL | NYAGVVFILF | WGLAPEYWMV | ITALRDSKHT | FDTTPWPTHV | TLDNFRDALA |
| TDKGNNFLAA | IGNSLVISVT | TTAIAVLVGV | FTAYALARLE | FPGKGIVTGI | ILAASMFPGI |
| ALVTPLFQLF | GDLNWIGTYQ | ALIIPNISFA | LPLTIYTLVS | FFRQLPWELE | ESARVDGATR |
| GQAFRMILLP | LAAPALFTTA | ILAFIATWNE | FMLARQLSNT | STEPVTVAIA | RFTGPSSFEY |
| PYASVMAAGA | LVTIPLIIMV | LIF | | | |
| > RXA02394 (1-1311, translated) 437 residues | | | | | |
| MLSPAAVAAL | ILVIGIVVLI | IASVPVAIAI | GLPSLFAAMA | VLGPENAAQA | VAQRMFTGTN |
| SFTLLAIPFF | VLAGLLMNSG | GIATRLIDAA | KVLVGRMPAS | MANTNIAANG | LFGAVSGAAV |
| ASASAVGTVM | TPKMKEEGYS | RAYAAAVNVA | SAPAGMLIPP | SNTFIVYSLV | SSTSIAALFM |
| AGVGPGLLWI | LACVIVGTWL | ARKENYKREQ | IHPTFKQSLV | VLWRALPSLL | MIVIVVGGIL |
| LGWFTPTESA | AIAVVYCLVL | GFIYRTIKVG | DLADILLKAT | RTTSIVMLLI | AVSAALSWVM |
| AFAKIPQMIS | DALLSVSDSK | VVILLIMMFI | LLLIGTVMDP | TPAILIFVPI | FLPVVTELGV |
| DPVHFGAMVV | MNLSVGVITP | PVGNVLFVGS | QVAGLRVETV | IRRLWPYLIA | ITVALFVVVF |
| VPQISIWLPT | TMGLMGG | | | | |
| > RXA02402 (1-744, translated) 248 residues | | | | | |
| VSKTEEGRSA | AIIIYAFPTF | ILLGATIAFI | FPEPFIPLTN | YTNIFLTIIM | FTMGLTLTVP |
| DFQMVLKRPL | PILIGVVAQF | VIMPFLAIVV | AKMFNLNPAL | AVGLLMLGSV | PGGTSSNVIA |
| FLARGDVALS | VTMTSVSTIV | SPIMTPFLML | MLAGTETAVD | GGGMAWTLVQ | TVLLPVIIGL |
| VLRVFLNKWI | DKILPILPYL | SILGIGGVVF | GAVAANAERL | VSVGLIVFVA | VIVHNVLGYV |
| VGYLTGRV | | | | | |
| > RXA02422 (1-435, translated) 145 residues | | | | | |
| VSTLISEPEV | DKLRKRAKRS | RRTEWWLAAA | LLAPNLLLLA | IFTYRPLLDN | FRLSFFNWNI |
| SSPTSTFIGF | DNYVEFFTRS | DTLQVVLNTV | IFTACAVIGS | MVLGLLLAML | LDQKLFGRNF |
| VRSMVFAPFV | ISGAAIGGAF | QFVFD | | | |
| > RXA02438 (1-759, translated) 253 residues | | | | | |
| MTDLIQLREV | SKKYGAFQAL | NDINLNVRAG | EVTCVLGDNG | AGKSTLIKIL | SGLHPATSGE |
| VIVAGDVVNF | GSPRDALDAG | IATVYQDLAV | VGQMSVWRNF | FLGQELTGRF | GVLKQEEMRR |
| ITDEQLREMG | IELRDVDVPV | ASLSGGQRQV | VAIARAIYFG | ARVLILDEPT | AALGVKQSGM |
| VLRFIAAARD | RGIGVIFITH | NPHHAYLVGD | HFILLNLGKQ | VMDKSRAEVE | LEELTLAMSG |
| GGELDSLSHE | LKR | | | | |
| > RXA02439 (1-1023, translated) 341 residues | | | | | |
| MTKIKSGEAS | TSIVERALKR | PELTSLLGAV | LVFTLFMVVA | PAFRSWDSMA | TVLYASSTIG |
| IMAVAVGLLM | IADEFDLSTG | VAVTTAALAA | SMFSYNLWLN | TWVGALIALV | ISLAIGFFNG |
| FLVVKTKIAS | ELITLATFLM | LQGINLAVTK | LISGTVATPT | IADMEGFPSA | RAVFASSIPI |
| FGVNIRITVF | WWLLFVIVGT | FVLEKTRIGN | WIFAVGGDEE | AARAVGVPVR | GVKIGLFMFV |
| GFAAWFVGMH | NLFLFDSIQA | GQGVGNEELY | IIAAVIGGIS | MTGGRGTVVG | TMIGALIEGM |
| TNQGIVYAGW | NPDWEMFFLG | GTLLLAVLLN | HRFERENKER | S | |
| > RXA02441 (1-657, translated) 219 residues | | | | | |
| MAELSVRNLT | CTYGNHIALN | NITARFPTGK | ITALIGSNGS | GKSTLLETLA | GMLAPRSGSI |
| NNLVPEIAFV | PQRSHVSHNL | PITIRQTVSM | GRWSAKKNWQ | RLTAADCNIV | DSCLDRLEIS |
| GLADRPLGEV | SGGQRQRALI | AQGLAQQAPL | LLLDEPLAAV | DSHAASLIED | VINQQRNQGT |
| TIILATHDLD | QAHQADQIIA | LEKGIIKPQR | KATESIKKR | | |
| > RXA02442 (1-849, translated) 283 residues | | | | | |
| MKFFTDALIV | PFDVSFISRA | LVAGCLAAIL | CSLIGTWVIL | RRLTFFGDAM | SHGLLPGVAT |
| ASLLGGNLMF | GAAISALIMS | AGVVWTSRKS | SLSQDVSIGL | QFITMLSLGV | VIVSHSDSHA |
| VDLTSFLFGD | ILGVRPSDIF | IIAIATVLGG | LTIFLEHRQF | TALAFDERKA | HTLGLNPRFA |
| HLLMLALIAL | ATVVSEQVVG | TLLVFGLLIG | PPATAALLVQ | DKASISLIMI | VASLLGCAEI |
| YLGLLISWHA | STAAGATITL | LSAAIFFATL | LTKSAISRLN | FTA | |
| > RXA02447 (1-270, translated) 90 residues | | | | | |
| WVWLAEIFPV | RMKGIGTGIS | VFCGWGINGV | LALFFPALVS | GVGITFSFLI | FAVVGVIALA |
| FVTKEVPETR | GRSLEELDHA | AFTGQIFKKA | | | |
| > RXA02451 (1-1524, translated) 508 residues | | | | | |
| MNTDTTQDGV | SPEPSDPHLG | SEVAETHREK | KFFGQPWGLA | NLFGVEMWER | FSFYGMQSIL |
| AFYLYYSVTD | GGLGMNQTAA | LSIVGAYGGF | VYMTSLVASF | IADRVLGSER | TLFYSAIIVM |
| LGHIALALIP | GYTGLSIGLV | LIGLGSGGVK | TAAQVVLGQL | YSRTDTRRDA | GFSIFYMGVN |
| LGGLFGPLIT | NALWGWGGFH | WGFGIAAVGM | ALGLIQYVAM | RKTTIGAAGH | TVPNPLPKNE |
| YARWIIGAVV | VVAAVVALIA | TGIIKLEWLS | NITAAIALIA | AIALLAQMYV | SPLTTAAEKS |
| RLLGFIPMFI | GGVLFFAIFQ | TQFTVLAVYS | DTRLDRNFFG | IDLPPGLINS | FNPIFIIIFS |
| GIFATLWTKL | GAKQWSTAVK | FGVANIVIGC | ALFFFLPFAG | GAENSTPMAL | IIWVYFLFTI |
| AELLLSPVGN | SLATKVAPEA | FQSRMFAVWL | MAVSMGTSLS | GTLGGYYDPT | DAGSEKVFFI |
| TVGVAAIVLG | AIVIAAKGWV | LKKFIDVR | | | |
| > RXA02491 (1-1254, translated) 418 residues | | | | | |
| MRVAMISMHT | SPLQQPGTGD | SGGMNVYILS | TATELAKQGI | EVDIYTRATR | PSQGEIVRVA |
| ENLRVINIAA | GPYEGLSKEE | LPTQLAAFTG | GMLSFTRREK | VTYDLIHSHY | WLSGQVGWLL |
| RDLWRIPLIH | TAHTLAAVKN | SYRDDSDTPE | SEARRICEQQ | LVDNADVLAV | NTQEEMQDLM |
| HHYDADPDRI | SVVSPGADVE | LYSPGNDRAT | ERSRRELGIP | LHTKVVAFVG | RLQPFKGPQV |
| LIKAVAALFD | RDPDRNLRVI | ICGGPSGPNA | TPDTYRHMAE | ELGVEKRIRF | LDPRPPSELV |
| AVYRAADIVA | VPSFNESFGL | VAMEAQASGT | PVIAARVGGL | PIAVAEGETG | LLVDGHSPHA |
| WADALATLLD | DDETRIRMGE | DAVEHARTFS | WAATAAQLSS | LYNDAIANEN | VDGETHHG |
| > RXA02507 (1-1401, translated) 467 residues | | | | | |
| MSEQLQGVTH | SESTPGKTPK | RAALSSWIGS | ALEYYDFAVY | GTAAALVLNH | LFFPADTSPG |
| IAILAAMGTV | GVAYVVRPLG | ALIMGPLGDR | YGRKFVLMLC | LFLIGASTFA | VGCLPTFDQV |
| GYLAPALLVL | CRVIQGLSAS | GEQSSAISVS | LEHADERHRA | FTASWTLHGT | QFGTLLATGV |
| FIPFTLFLSE | DALMSWGWRV | PFWLSAAVVL | VAFLIRRGLE | EPPAFRENKE | AVAGAASPLA |
| MTLRYHKAAV | ARVAIAAMIN | SVNIVFTVWA | LSFATNIVGL | DRSTVLLVPV | VANLVALIAI |
| PLSGMLADRI | GRRPVFIMGA | IGGGLAMNGY | LGAIYSGNWT | MIFFMGVLMS | GLLYSMGNAV |
| WPAFYAEMFP | TSVRVTGLAL | GTQIGFAVSG | GFVPVIASAL | AGDQGDQWMK | VSIFVGVVCV |
| ISALVAMTAK | ETKALTLDEI | DALHTAGGEA | ADLAAASKAS | EAQLAAQ | |
| > RXA02515 (1-756, translated) 252 residues | | | | | |
| MSTLEIRNLH | AQVLPSDESA | EPKEILKGVN | LTINSGEIHA | IMGPNGSGKS | TLAYTLGGHP |
| RYEVTAGEVL | LDGENILEME | VDERARAGLF | LAMQYPTEIP | GVSVANELRS | AATAIRGEAP |
| KLREWVKEVR | TAQEALAIDP | EFSNRSVNEG | FSGGEKKRHE | VLQLDLLKPK | FAIMDETDSG |
| LDVDALRIVS | EGINSYKQET | EGGILMITHY | KRILNYVKPD | FIHVFANGQI | VTTGGAELAD |
| KLEADGYDQF | IK | | | | |
| > RXA02562 (1-720, translated) 240 residues | | | | | |
| MFLTKVSLLD | HPESLPGYLS | SLAIVEYLHE | QPLEFRAPIT | VITGENGVGK | STLVEALAVG |
| MRLNPSGGSR | HANFGREGDI | VSSLHQSLKL | VRRENPRDAF | FFRGETMYNV | ASYYEELMGE |
| KNMHDLHKMS | HGESVFAVID | RRFNNQGFFV | LDEPEAGLSM | LRQLELLGKL | GNLARGGAQI |
| IMATHSPILL | AIPGAEILEI | TSSGVAKVNF | EDAEAVRAAR | EFVADPRGTA | AFLTAEEDHQ |
| > RXA02595 (1-651, translated) 217 residues | | | | | |
| VIVVAMASIM | ACLKAARLNN | PMKILLLCWR | DTTHPQGGGS | ERYLERVGEF | LADQGHEVVF |
| RTAGHTDAPR | RSFRDGVRYS | RSGGKESVYP | KAWVAMMLGR | VGIGTFSKVD | VVVDTQNGIP |
| FFGKFFSGKP | TVLLTHHCHK | EQWPVVGRVL | AKVGWLIESQ | IAPRAYKTAP | YVTVSEPSAE |
| ELIALGVDQQ | RIHIVRNGVD | PVPLHTPKLD | RDGQHAV | | |
| > RXA02597 (1-1788, translated) 596 residues | | | | | |
| LPEQDLTTLA | NDWLQAFEKA | TASSSPDEAA | TAVVQLFEDE | GYWRDLLAFT | WNLTTAEGAD |
| EIAEMIRNTW | PSSIFRNVEL | KGEPADEGDG | VTRVHFSCES | ADFKCTGIVR | LRNGKAWTLL |
| TSARELLEHP | EPKGRNREMG | VVHGQNEDTR | NWTDRKNDRQ | AALGVTEQPY | TLIIGGGQGG |
| IALGARLKRL | GVPALIIDKA | SRPGDQWRSR | YHSLCLHDPV | WYDHLPYIPF | PDHWPVFTPK |
| DKMGDWLEHY | VGIMDLDYWT | NTECLRASYN | EDTKQWDVTV | NRDGAESTLH | PTQLVMATGM |
| SGSPNKPTLP | GQDKFQGEIR | HSSEHPGGDV | DRDKNVVVLG | ANNSAHDICA | DLYSNGAKPV |
| MIQRSSTHIV | RSDSLMREVF | GPLYSEDAVE | AGIDTDTADL | LFASWPYKVL | PGVQKQAFDK |
| IREDDKEFYD | KLENAGFLLD | FGDDDSGLFL | KYLRRGSGYY | IDVGASELVA | DGKIPVRSNV |
| SIEDVKENSV | VLTDGTELPA | DVIVLATGYG | NMNNWVAQLV | DQETADKVGP | CWGLGSETTK |
| DPGPWEGELR | NMWKPTNVDS | LWFHGGNLHQ | SRHYSRYLSM | QLKARYEGMN | TPVYSK |
| > RXA02605 (1-495, translated) 165 residues | | | | | |
| VACPWAGTAA | LNLAAKHPDQ | FRQAMSWSGY | LNTTAPGMQT | LLRVAMLDTG | GFNVNANYGS |
| IINPRRFEND | PFWNMGGLAN | TDVYISAASG | LWSPQDDGVR | VDHRLTGSVL | EFVAMTSTRI |
| WEAKARLQGL | NPTADYPMYG | IHGWAQFNSQ | LERTQGRVLD | VMNAW | |
| > RXA02614 (1-729, translated) 243 residues | | | | | |
| MTATLSLKPA | ATVRGLRKSY | GTKEVLQGID | LTINCGEVTA | LIGRSGSGKS | TILRVLAGLS |
| KEHSGSVEIS | GNPAVAFQEP | RLLPWKTVLD | NVTFGLNRTD | ISWSEAQERA | SALLAEVKLP |
| DSDAAWPLTL | SGGQAQRVSL | ARALISEPEL | LLLDEPFGAL | DALTRLTAQD | LLLKTVNTRN |
| LGVLLVTHDV | SEAIALADHV | LLLDDGAITH | SLTVDIPGDR | RTHPSFASYT | AQLLEWLEIT |
| TPA | | | | | |
| > RXA02616 (1-711, translated) 237 residues | | | | | |
| LQKHTRGGKH | RKQTTSPVTK | GGVAFVAVAT | GAVSTAGAGG | AVAAQASNQP | VEVNFELTAN |
| DTTDLVAGSS | APQILSIAEF | KPVVNLGDQI | VKTIQYNADR | IQADLDARGP | SVVRPAEGSY |
| TSGFGARWGT | NHNGVDIANA | IGTPILAAMD | GTVIDAGPAS | GFGNWVRLQH | EDGTITVYGH |
| METVEVTVGQ | TVKAGERIAG | MGSRGFSTGS | HLHFEVYPAG | GGAVDPAPWL | AERGITL |
| > RXA02627 (1-843, translated) 281 residues | | | | | |
| DVTVESQPER | VVALGWGDAE | AALEFGVQPV | GASDWLAFGG | EGVGPWIEDS | AYDEAPEIIG |
| TMEPEYEKIA | ALEPDLTLDV | RSSGDQERYD | KLSSIALTIG | VPEGGDSYLT | PRAEQVTMIA |
| TALGQAERGE | EVNAEYEQLT | ADIRAAHPGW | PEKTAAAVSA | TATSWGAYIK | GSNRVDTLLD |
| LGFQENPELA | KQQPGDTGFS | IKFSEETFGV | VDSDLVVGFA | IGMTPEEMAE | QVPWQMLTAT |
| RDGRSFVMPR | EISNAFSLGS | PQSTRFALDA | LVPLLEEHAG | E | |
| > RXA02628 (1-405, translated) 135 residues | | | | | |
| MLEGFRDFVL | RGNVIELAVA | VVTGTAFTAI | VTAFSESIIN | PLIASIGSTE | VEGLGFHIRA |
| GNAATFVDFG | AVITAAINFL | IIAAIVYFVL | VAPMNKLSET | LAKRKGVEED | ETPASIEAEL |
| LTEIRDLLQE | QKRLQ | | | | |
| > RXA02650 (1-579, translated) 193 residues | | | | | |
| MVNVTSKDAG | ANVTPMSKKE | KRTTVKQVVA | LMAAIVVVIA | SLDQIVKQIM | LSWLEPGVPV |
| PIIGDWFRFY | LLENPGAAFS | MGGENSTWIF | TTIQLSFVIG | IAIYAPRIKH | KWIAAGLALV |
| AGGALGNVLD | RLFRDPSFFF | GHVVDYISVG | NFAVFNIADA | SISCGVVVFL | IGMFLEDREN |
| AQHAKATDEK | DEA | | | | |
| > RXA02660 (1-639, translated) 213 residues | | | | | |
| MIIGVTLLVF | IVMSFSPADP | ARLALGESAS | PEALEAYREA | NGLNDPMMVR | YFDFILGMLK |
| GDLGTSSGGV | AVTDIVARAF | PITLQLTFWG | LIIAVVVALI | LGVIAALYRD | RWPDQLIRVV |
| SIAALATPSF | WLAILLIQWL | GTIPGAWGFF | PALVTRWVPF | SEDPATYFNN | IALQRLRWQS |
| PLQVLWPALF | VPPWWKNWTR | TTSAQQSVQD | PQN | | |
| > RXA02661 (1-219, translated) 73 residues | | | | | |
| VIGLRVGSLM | GGAVIIEIIF | NIQAMGQLIL | DGVTRNDVYL | VQGVTLTVAI | AFIIVNIAVD |
| LLYVLVNPRI | RSI | | | | |
| > RXA02663 (1-1395, translated) 465 residues | | | | | |
| MAPILVFATV | LVADAIVFEA | SLSFINAGVK | PPSPSWGNIL | ADGKALLLSG | AWWPTFFPGL |
| MILLTVLCLN | ILSEGLTDTL | ASPKPKPVSA | SAKKALKKEE | SGEKEGSGIV | LGHTTREEAN |
| ASLLASLAAL | STSENNSNNR | LIFDGNPTPL | LEVRDLKISF | PNAHGDINIV | DGVNFTVAPG |
| QTMGLVGESG | CGKSITAMSI | MGLLPPTAKI | EGEILFDGKN | LLDLKPDELN | ALRGHEIAMI |
| YQDALSSLNP | SMLISAQMKQ | LTRRGGKRSA | EELLELVGLD | PKRTLQSYPH | ELSGGQRQRV |
| LIAMALTRNP | RLLIADEPTT | ALDVTVQQQV | VDLLNELREK | LGFAMIFVSH | DLALVARLVH |
| KLTVMYAGQV | VEQGTTREIL | IDPRHEYTRG | LLGSVLSIEA | GVDRLYQVPG | TVPSPKEFVA |
| GDRFAPRSEF | PELGLDQKPV | LRPITGTEHA | YAATDELLAA | KGEQR | |
| > RXA02664 (1-660, translated) 220 residues | | | | | |
| VGESGCGKST | LARVMVGLQP | VTSGEVLFKG | KPMKPRGAQR | KELGSSVSVV | FQDPATSLNP |
| RMTVREQLLD | PLRVHKVGDE | ASRNQWVSEL | ISMVGLPQSA | LEVLPRQVSG | GQRQRVAIAR |
| ALALKPDIIV | ADEPTSALDV | SVRAQVLNLL | LDLKTELGLG | LVFISHDINT | VRYVSDRIAV |
| MLAGEIIEEN | TTSEIFNNAQ | QDYTRTLLEA | TPSLLNKTRL | | |
| > RXA02684 (1-864, translated) 288 residues | | | | | |
| VLAVGLVLVF | VVTLWADSKL | NRVDATPATQ | VANTAGTNWL | LVGSDSRQGL | SDEDIERLGT |
| GGDIGVGRTD | TIMVLHMPRT | GEPTLLSIPR | DSYVNVPGWG | MDKANAAFTV | GGPELLTQTV |
| EEATGLRIDH | YAEIGMGGLA | NMVDAVGGVE | MCPAEPMYDP | LANLDIQAGC | QEFDGAAALG |
| YVRTRATALG | DLDRVVRQRE | FFSALLSTAT | SPGTLLNPFR | TFPMISNAVG | TFTVGEGDHV |
| WHLARLALAM | RGGIVTETVP | IASFADYDVG | NVAIWDEAGA | EALFSSMR | |
| > RXA02728 (1-813, translated) 271 residues | | | | | |
| MAIVSLDNVT | VSIEGKKLLD | AVSLKAYPGE | VLGLIGPNGA | GKSTLLSVLS | GDRLPDSGEV |
| NVGGLDPATA | AASDMARVRA | VMLQDVSVAF | SFLVWDVVEM | GRRPWQKAST | PEEDHEIIEA |
| ALAATSVSHL | AEREITTLSG | GERARVALSR | VLAQQTPIVL | LDEPTAAMDI | SHQEQTLGTA |
| RALAAAGAAV | IVVLHDLNAA | AAYCDSIVCL | SDGRVIASGS | VDQVYSTETL | SRVYGWPIRV |
| DHSGKYVRVE | PDRSEANLPS | VLQVKNTVSP | A | | |
| > RXA02750 (1-816, translated) 272 residues | | | | | |
| MAVLFSIMGA | LILLVLYVLF | LGKLQIDGLM | VDLPDSARDD | VEGFVFNWVF | SGILITSAIT |
| VPQAALGVLV | EDRTRGGIKD | FLVAPVSRTT | LTVSYIEAAV | IVAMTILIFE | IVVGSIGLAI |
| LGHFSMSIAR | VLELVVALLL | LTLVFSAIAA | FLITLVKSQG | GMSALSSLVG | TLAGFLSAAY |
| IPPIALPEAV | TNVLNFLPFT | PAGMLIRQIV | VAPALDAISL | PPEAFDIFQF | GYGLKLEMFG |
| EPVSTWVAVG | IVASWGVVFG | LIAAFKMKSV | VR | | |
| > RXA02761 (1-201, translated) 67 residues | | | | | |
| MMDGINRRTT | LITGYSLTTI | SHVLIGIASV | AFPVGDPLRP | YVILTLVVVF | VGSMQTFLNG |
| SYLGYAL | | | | | |
| > RXA02762 (1-285, translated) 95 residues | | | | | |
| MLSELFPLAM | RGFAIGISVF | FLWIANAFLG | LFFPTIMEAV | GLTGTFFMFA | GIGVVALIFI |
| YTQVPETRGR | TLEEIDEDVT | SGVIFNKDIR | KGKVH | | |
| > RXA02769 (1-711, translated) 237 residues | | | | | |
| TVVPVYLAEL | APLEIRGSLT | GRNELAIVTG | QLLAFVINAL | IAVTLHGVID | GIWRIMFAVC |
| ALPAVALFLG | MLRMPESPRW | LVNQGRYDDA | RRVMETVRTP | ERAKAEMDEI | IAVHSENNAA |
| LPGVKQSSGQ | ASGQVSSKHT | HMSIGEVLSN | KWLVRLLIAG | IGVAVAQQLT | GINAIMYYGT |
| RVLEESGMSA | EMAVVANIAF | GAVAVIGGLI | ALRNMDRLDR | RTTFIIGLSL | TTTFHLL |
| > RXA02795 (1-1095, translated) 365 residues | | | | | |
| IDVSLPERTA | SAYPHELSGG | QRQRALIAMA | LANDPDLLIC | DEPTTALDVV | VQKQIVDLLL |
| RLTKERGTAL | LFITHDLGLI | ARTCERLLVM | KSGETVERGD | TEAILRSPAH | SYTQQLLDAS |
| ILDQPEIASD | SGAPVVIDVE | EASKSEKETT | ALHKVSLAVR | KGDLLGIVGG | SGSGKTTLLK |
| LIAGLDKPTT | GTVAVTGGVQ | MVFQDPQSSL | NPRMKIKDIV | AEPLLGWNAA | EKTTRVAEVI |
| TQVGLSPDVL | DRYPHEFSGG | QRQRISIARA | LAIKPAILLA | DEPVSALDVS | VRKQVLDLLQ |
| QLVEEYGITL | VFVSHDLAVV | RHLCTTVWVM | EQGRVLEQGP | IDSVYDHPQT | EYTKELLDAV |
| PRLSL | | | | | |
| > RXA02808 (1-258, translated) 86 residues | | | | | |
| FYEGILPVLA | ESASHFGIEP | VEMARASITG | QPVHMQSPLV | PAILLLVSLA | NVNLGDHHKK |
| VLWRACIVSI | AMLAVALFIG | VVPLSA | | | |
| > RXA02863 (1-975, translated) 325 residues | | | | | |
| MKKSLIAIVA | SALVLSGCTS | DSSDSSGTSG | TVETTSITTS | VAAADGAFPR | TVTLDDSSIT |
| LESKPERIAV | LTPEAASLVL | PITGADRVVM | TAEMDTADEE | TAALASQVEY | QVKNGGSLDP |
| EQVVAGDPDL | VIVSARFDTE | QGTIDILEGL | NVPVVNFDSD | AWGDIDAITK | HLEIVGELVG |
| EEDKAAEAIA | EIDANRIDID | KPATSPTVLT | LMQRGPRQMV | MPESAMLNGL | IREAGGTPVV |
| DSLGAVGTIT | ADPEQVVAMA | PEIIIIQDFQ | GKGRENFANF | LSNPALANVP | AIENDKIFYA |
| DTVTTGVTAG | TDITTGLQQV | AEMLS | | | |
| > RXA02864 (1-780, translated) 260 residues | | | | | |
| MPQLVEIRDL | NVEFPSRHAV | KNVSFSAPAG | KVTALIGPNG | AGKSTALSAI | AGLVESTGEV |
| MVGGSGVASK | SAKARARLLS | LVPQNTELRI | GFSARDVVAM | GRYPHRGRFA | VETDADRRAT |
| DDALRAINAL | DIAEQPVNEL | SGGQQQLIHI | GRALAQDTAV | VLLDEPVSAL | DLRHQVEVLQ |
| LLRARANSGT | TVIVVLHDLN | HVARWCDHAV | LMADGEVVSQ | GDIREVLEPA | TLSTVYGLPI |
| AVRDDPETSS | LRVIPHPNPF | | | | |

>RXN00001 TRANSLATE of: rxn00001.seq check: 7420 from: 1 to: 1128
MATVTFKDASLSYPGAKEPTVKKFNLEIADGEFLVLVGPSGCGKSTTLRMLAGLENVTDG
AIFIGDKDVTHVAPRDRDIAMVFQNYALYPHMTVGENMGFALKIAGKSQDEINKRVDEAA
ATLGLTEFLERKPKALSGGQRQRVAMGRAIVRNPQVFLMDEPLSNLDAKLRVQTRTQIAA
LQRKLGVTTVYVTHDQTEALTMGDRIAVLKDGYLQQVGAPRELYDRPANVFVAGFIGSPA
MNLGTFSVKDGDATSGHARIKLSPETLAAMTPEDNGRITIGFRPEALEIIPEGESTDLSI
PIKLDFVEELGSDSFLYGKLVGEGDLGSSSEDVPESGQIVVRAAPNAAPAPGSVFHARIV
EGGQHNFSASTGKRLP
>RXN00099 TRANSLATE of: rxn00099.seq check: 3872 from: 1 to: 1173
VKNPRLIALAAIILTSFNLRTAITALAPLVSEIRDDLGVSASLIGVLGMIPTAMFADAAF
ALPSLKRKFTTSQLLMFAMLLTAAGQIIRVAGPASLLMVGTVFAMFAIGVTNVLLPIAVR
EYFPRHVGGMSTTYLVSFQIVQALAPTLAVPISQWATHVGLTGWRVSLGSWALLGLVAAI
SWIPLLSLQGARVVAAPSKVSLPVWKSSVGVGLGLMFGFTSFATYILMGFMPQMVGDPQL
GAVLLGWWSILGLPLNILGPWLVTRFTNCFPMVVIASVMFLIGNGGFCLAPDVAPWLWAT
LSGLGPLAFPMALTLINIRAETSAGASALSSFGQGLGYTIACFGPLLTGFIVDATGSFRT
IFVLFAVATLFVIRGGYFATRQVYVEKLLNR
>RXN00193 TRANSLATE of: rxn00193.seq check: 1918 from: 1 to: 594
KAFXQREGFISAFGFTVLVVIVSVITVNIFAFLLAWLLTRKLRGTNFFRTVFFMPNLIGG
IVLGYTWQTMINAVLSHYATTTSADWKFGYAGLIMLLNWQLIGYMMIIYIAGLQNVPPEL
IEAAELDGVNKWEMLRHVTIPMVMPSITICLFLTLSNSFKLFDQNLALTNGAPGGQTEMV
ALNIINTLFNRMNVEGVG
>RXN00378 TRANSLATE of: rxn00378.seq check: 9591 from: 1 to: 2610
VDKAVNTAISDAKTAALKAGVGLNRATASEEEEDLSSSIKVSLAFELEGLSNAPSLMVVE
KALEKIPGVSADLIYPSQTAWITATDRVHPETLIEVFEQFGIKAHLSNSSLLRRHQQLSA
EVNREARLDRYRSRMDAKRISPRVRRHNRQEMVHAVRARESGWIKRRNHTTSQHEDPMSG
DVLFTARALITPKRLWVSLPFALIVLALSLNPSWQFDYWQWLSAVLAIPVVVWGAWPFHR
AAAGGIRRGISALDATSSIAIAAAYAWSIAMLLFETPGGKSWRSYPSWFAFDHGTLTQNE
IYFDVACGITVLLLAGRLLTRRRSQSSLLAELGRLQIDPQRIVTVVRKHRLKRVVQELNI
PVQEVRVNDDVKVPPNTTIPVDGTVIGGGSRIAASIIMGQDQRDVKVNDKVFAGSLNLES
EIKVRVIRTGHRTRIAAVHRWVKEATLKENRHNRAAIRSAGNLVPITFTLAVVDFCLWAL
ISGNINAAFTTTLAVLACVAPVALALSAPLATRNSIEAAARHGILVRSGEIFRVLDDVDT
AVFNRVGTLTDGEMTVETVTADKGEDPELVLRVAGALAMESHHAISKALVKASREARDTG
AGGEDVPHWIEVGNVEITEAGSEQATIELPLIKPSGEKIMRTTEALLWRPRSMTEVREHL
SPRLVAAATSGGAPLIVRWKGKDRGVITLSDHVRSDSSDAIIAIEEQGIETMMLSRDTYP
VARRYADSLGITHVLAGIAPGKKAQVVRAVHTRGSTVAMIGDESVMDCLKVADVGVLMGV
DRPSDLRDDSDDPAADVVVMREEVMSVPTLFKLARRYAKLVNGNIALAWIYNGVAMVLAV
SGLLHPMAATVAMLASSLLIEWRSGRARKY
>RXN00412 TRANSLATE of: rxn00412.seq check: 7568 from: 1 to: 1080
VSHTASTPTPEEYSAQQPSTQGTRVEFRGITKVFSNNKSAKTTALDNVTLTVEPGEVIGI
IGYSGAGKSTLVRLINGLDSPTSGSLLLNGTDIVGMPESKLRKLRSNIGMIEQQFNLFQS
RTAAGNVEYPLEVAKMDKAARKARVQEMLEFVGLGDKGKNYPEQLSGGQKQRVGIARALA
TNPTLLLADEATSALDPETTHEVLELLRKVNRELGITIVVITHEMEVVRSIADKVAVMES
GKVVEYGSVYEVFSNPQTQVAQKFVATALRNTPDQVESEDLLSHEGRLETIDLTETSGFF
AATARAAEQGAFVNIVHGGVTTLQRQSFGKMTVRLTGNTAAIEEFYQTLTKTTTIKEITR
>RXN00431 TRANSLATE of: rxn00431.seq check: 340 from: 1 to: 789
MVSIDTYNACVDFPIEDAKSRSMKKAFLGAAGGAIGRNQDNVVVVEALKNVNLHLREGDR
VGLVGHNGAGKSTLLRLLSGIYEPTRGSADIRGRVAPVFDLGVGMDPEISGYENIIIRGL
FLGQTRKQMKAKMEEIADFTELGEYLSMPLRTYSTGMRIRLALGVVTSIEPEILLLDEGI
GAVDAAFMAKARDRLQALVERSGILVFASHSNDFLAQLCNTALWVDHGQIREAGLVPDVV
EAYEGKGAGDHVRRLLTRMEEEK
>RXN00444 TRANSLATE of: rxn00444.seq check: 7535 from: 1 to: 837
MVLAQTKKARRSENHILPGWLLIPATLAMLLIIGPIEALLLQIPWDRSWELLTAPESLGT
ARLSIGTALFSTALCAIVGFPLALALHLYERSHPRVTSVLTVLVYAPLVLSPVVSGLALT
FLWGRRGFLGSWLDQVGLPIAFTTTAVVEAQVFVALPFFISTVTTALRGIPKQFEEIAAT
EGATRWEIMHKMIIPLAMPGIFTGMILGFARALGEYGATLTFAGNIAGVTRTIPLHIELG
LSSNDMDKALGAVIMLLAVYVLIIGAIGALRLFSKVRKV
>RXN00466 TRANSLATE of: rxn00466.seq check: 8825 from: 1 to: 996
VQSRLSKILRSSVVGVAVLALLAGCSNNADDTDADSTSTGNSAFPVSIEHEFGTTTIDDV
PERVVTLGVTDADIVLALGTVPVGNTGYKFFENGLGPWTDELVEGKELTLLDSDSTPDLE
QVAALEPDLIIGVSAGFDDVVYEQLSDIAPVVARPAGTAAYAVAREEATNLVARAMGQSE
KGQELNEETDALIQAARDENPSFDGKTGTVILPYQGKYGAYLPGDARGQFLDSLGISLPE
AVLSRDTGDSFFVDVPAESVKDVDGDVLLVLSNDENLDITAENPLFETLNVVQKDAVIVA
TTEERGAITYNSVLSVPFALEHLAPRIAEALK
>RXN00523 TRANSLATE of: rxn00523.seq check: 9218 from: 1 to: 1026
MSLSHQLKRQRASRNSRRWLIVAALGVVTLGIFAFSLMWGEVFYGPAQVLKVLSGQQVPG
ASYSVGVLRLPRAVMGLTAGLAFGAAGVIFQTVLRNQLASPDIIGISSGASAAGVICIVF
FGMSQSAVSAISLCASLAVALLIYLVAYRGGFSATRLILTGIGIAAIMLNSLVSYSLSKAD
SWDLPTATRWLTGSLNGATWDRAMPLIVTTVVLIPLLVANARNVDLMRLGNDSAVGLGVA
TNRTRVIAIIAAVALIAVATAACGPIAFVAFVSGPIAARILGSGGSLIIPSALIGGLIVL
IADLIGQYFLGTRYPVGVVTGAFGAPFLIYLLIRSNRAGVTL
>RXN00525 TRANSLATE of: rxn00525.seq check: 5915 from: 1 to: 1263
MSLAESILLALTSLRSNKMRALLTLLGVIIGIASVIGILTIGKALQDQTLNSLESLGAND
LSAQVEERPDEDSPEPDMFAFSGAANSSGNLIPEETVDTLRDRFAGSITGISVGGMGTQG
TLIGDTADLKSDLLGVNEDYMWMNGVEMNYGRAITQDDVAAQRPVAVIAPDTFNTLFDAN
PNLALGSEVAFELNGQETFLRVIGVYKEAAAGGLVGSNPTVHTYTPYTVANDITHTEDGL
NTLSIRAAQGVDQDSLKGSLQTYEDALYANNDSHHVAMLDFRKQIEEFNTILGAMSLGIS
AIGGISLLVGGIGVMNIMLVSVTERTREIGVRKALGARRRDIRLQFVVEAMIICFIGGIL
GVLLGGILGLIMSSAIGYISLPPLSGIVIALVFSMAIGLFFGYYPANKAAKLDPIDALRY
E
>RXN00702 TRANSLATE of: rxn00702.seq check: 9529 from: 1 to: 1707
MSAPFSARTAWSTDPVLELESVAASYYDDERTLAAPQISDVNLTLFEGEILLVVGRTGSG
KSTLLNAMSGAMPHATGGRLDGRVRVVGRDTRDFPPRMLSDVVGVVGQDPAASFITNTVE
EELAYSMEQLGLPPAVMRKRVEETLDLLGIAELRYVPLAELSGGEQQRVAIGAVLTTRPA
LIILDEPTSALDPNGAEDVLATVTKLAHDLAMTVVLAEHRIERVLQYVDRVAHVGADGHV
TVGTPEEIMADSDVAPPIVELGRWAGWAPLPLSIRDARAHSADMRKRLYQRGLVVNKLHN
HAVQPLLIAEDIMVDFPEIRAVDGVNLNLNSGEITVLMGRNGCGKSSLLWALQGSGTRNQ
GSVQVLDEAAGFSWTDPKTLKPAKRRNLVSMVPQTPTDILYESTVHAELARSDKDAAAPA
GTTREILDSLVPNIPDHLHPRDLSEGQKLSLALSIQLAAKPRVVFFDEPTRGLDYDGKKS
LARSFQQLADDGHAILVVTHDVEFSALCADRVLFMASGKIISDGTAVEILPASPAYAPQV
AKITAGIQEESHWLTVSAVKAALGHGEIS
>RXN00726 TRANSLATE of: rxn00726.seq check: 2288 from: 1 to: 591
NAGRLYVDGDLIGYRERDGVLYEISEKDAAKQRSDIGMVFQNFNLFPHRTVIENIIEAPI
HVKKQPESKARARAMELLEQVGLAHKADAYPVQLSGGQQQRVAIARAVAMEPKLMLFDEP
TSALDPELVGEVLRVMKQLADDGMTMLVVTHEMGFAHEVADQVVFMADGVVVEAGTPEQV
LDNPKEQRTKDFLSSLL
>RXN00732 TRANSLATE of: rxn00732.seq check: 6509 from: 1 to: 1647
NHLLLLPTVKADIIDNGVVTGDIGYIWHTGGIMLALTLVQVACAIAGVYFGSKLSMRVGR
DLRSAIFGKVVNFSEREMGQFGAPSLITRNTNDVQQVQMLVQMTSTLMISAPMLAIGGII
MAVRQDLGLSWLMVVSIPVLIIVVALIIVRMVPLFQTMQKRIDRINQIIREQLTGIRVIR
AFVREDVERERFTTASKDVADIGVRTGNLMALMFPAVMLIMNLSAVAVIWFGAFQVESGE
TQIGTLFAFLQYIMQILMGVMMAAFMFVMVPRAAVSADRIGEVLETTPSVQAPETPAQPS
TSAGEIVFNNATFAYPGADDPVLNNVSFRVAPGSTTAIIGSTGSGKTTLIGLVPRLFDVT
EGDVTVDGTDVREFEPLKLWDRIGLVPQKSFLFSGTIASNLRYGNEDATETQLWQALAIA
QAADFVREMPEGLDSEIAQGGTNVSGGQRQRLAIARALLKQPEIYIFDDSFSALDVSTDA
ALRRALSTNLPDATKLIVAQRVSTIRDADQIVVLDNGEVVGIGTHTNLLNTCGTYREIVE
SQETAQAQS
>RXN00759 TRANSLATE of: rxn00759.seq check: 3116 from: 1 to: 924
MLRYVGRRLLQMIPVFFGATLLIYALVFLMPGDPVQALGGDRGLTEAAAEKIRQEYNLDK
PFTVQYLLYIKGIFVLDFGTTFSGQPVIDVMARAFPVTIKLAIMALLFESILGIIFGVIA
GIRRGGIFDSTVLVLSLIVIAVPTFVIGFVLQFLVGVKWGLLPVTVGSNTSITALIMPAV
VLGAVSFAYVLRLTRQSVSENLRADYVRTARAKGMSGFNVMNRHVLRNSLIPVATFLGAD
LGALMGGAIVTEGIFGINGVGGTLYQAILKGEPTTVVSIVTVLVIVYIIANLLVDLIYAV
LDPRIRYA
>RXN00808 TRANSLATE of: rxn00808.seq check: 7354 from: 1 to: 1458
VLGTNVFGALAVMLFVRFLIPQPDASNFNAEISYLPAVGFAYLAFAIVAGMLVTFLMFRP
VLDWQRSPEDHDRNMVRNLVMRIPIYQAILCAVVWLIGIAIATLISASVSTSLALVVAFS
TLMAAAIVVLLTYLEAERLVRPVAASALARRFEDSTLEPPVSQRLRMTWLLTLGIPVMGI
LLLIWGYSQGIFGSDASGIMPAIAALAFASLVTGYLGNRLVVSSVVDPIRELQEAINRVR
RGENDVQVDIYDGSEIGVLQAGFNEMMRGLRERQRVRDLFGRYVGAEVAKRALEERPTLG
GEDRKVAVLFVDVIGSTTFAVNHTPEEVVEALNEFFEHVVEVVHRNKGVINKFQGDAALA
IFGAPLPLSDATGHALAAARELRAELKDLQLKAGIGVAAGHVVAGHIGGHARFEYTVIGD
AVNQAARLTEIAKTTPGRTVTNASTLREANEAEQARWTLMKSVELRGRSQMTQIARPIRP
TLADRS
>RXN00828 TRANSLATE of: rxn00828.seq check: 8544 from: 1 to: 453
VRGGLNTPPHKWRSADLAARIGTVFQDPEHQFVARTVRDELEIGPKIMKVDASERIEELL
DRLRLRHLENANPFTLSGGEKRRLSVATALVAAPKLLILDEPTFGQDPETFTELVTMLRE
LTDNGISIVSVTHDPDFIAALGDHHIEVSAK
>RXN00832 TRANSLATE of: rxn00832.seq check: 2297 from: 1 to: 1050
MPFSWLKPIDYARIFVGWASIFIIPLITLPSIIELALIVAVILFCAFGVVKMAERLAHIL
GDPFGSLILTLSIVIIEVILICAVMLGPADSTTAGRDSVMAVSMIIMGLVVGLCLLIGGL
RHGSMPHNGVGTPTYLVLIATFSVIAFAVPAFRGEYSTGQALVISTLTAVVYGFFLFRQM
GAQAGEFQEVEVAEKADDAAKWEVPFRGLILIITVLPIVLLSHDMATVMDEVLASLGAPV
AMAGLIIATIVFLPETITSLKAAWTGEIQRVSNLAHGAQVSTVGLTIPAVLVIGVITGQD
VVLGETPINLLLLGTTIAVTAIAFSSKKVSAVHGSVLLMLFGVYMMSMFA
>RXN00934 TRANSLATE of: rxn00934.seq check: 9723 from: 1 to: 1083
VRIGMVCPYSFDEPGGVQAHILDLARTFIAQGHEVQVLGPCSADTQVPDFVVRGGGSIPI
PYNGSVARLSFGPKMFKAVRTFLREGNFDVLHIHEPNSPSFSMAALRFAEGPIVATYHAS
SSGSKLLKAFLPVLSPMLEKVRAGIAVSEMARRWQVEQVGGDPVLIPNGVETSMFKAARQ
IEPNDPVEIVFLGRLDESRKGLDILLRALTRLDRPFTCTVIGGGTPREVAGINEVGRVSD
EEKAAILGRADIYVAPNTGGESFGIVLVEAMAAGCAVVASDLEAFSLVTDSEAAQPAGVL
EKTGSDADLAKKLQALIDDPSSRSTLIAAGLKRANAYDWSTVSTQVMAVYETIAIDKVRL
G
>RXN00939 TRANSLATE of: rxn00939.seq check: 3908 from: 1 to: 1236
MTRQKTQPFLEKESKYYTPGVMIAALAVGLITLNVELALTLLVIACPGALVISIPVSIVA
GIGRSAKDGVLIKGGEYLETSAKVDTVVVDKTGTLTNGRPELTNVDVLDPAYSDDEVLTL
AARAETASEHPLAEAIIRGAENRGLTVAMVEKAEPVAGRGIRADVDGATVAVGSADLLDH
TPDNTRILELNEQGRTAMYVGINGKAVGIVAVADTIRDDAPAAIRSLHNKGIRVVMATGD
AERVARNVAAELGVDEVRAELMPEDKLEIVKELQAQGRVVAMVGDGVNDTPALATADIGV
AMGAAGSPAAIETADTALMADKLPRLPYALGLAQRTVRTMRVNIGIALLTVTILLAGVLL
GGVTMSIGMLVHEASVLLVIAIAMLLLRPTLKEDKDKADVSTADAAKETLSA
>RXN00960 TRANSLATE of: rxn00960.seq check: 4118 from: 1 to: 1035
MARHCCSNRYASTVFSGLIAYGASQALYPWLLKDHQSVTEIDLDAGALQPYPNIENPPPF
EVMTALLLAECLGLGMAVIKSDTLFKVTRELERVVMKTITAFVIPLLPLFIFGIFLGMGM
NGGLLEIMSAFGKVLILAVVGTLLFLAIQFIIAGAVSKKNPWKLFKNNLPAYFTALGTSS
SAATIPVTYQQTLKNDVDVNVAGFVVPLCATIHLAGSMMKIGLFTFAVVFMYDMEVGVGL
SIGFLLMLGITMIAAPGVPGGAIMAATGMLASMLGFNTEQVALMIAAYIAIDSFGTAANV
TGDGAIAVIVNKFAKGQLHTTSPDEIEEDDRVAFDITPSDVEHHK
>RXN00980 TRANSLATE of: rxn00980.seq check: 2367 from: 1 to: 1794
MLADAFMIAAAIVAGWPIAQSAYQALRIRMVSIDLLVVVAAVGAMFINNYWESAAVTFLF
ALGKALERATMNRTRKALSDLVDAAPETATRLNADDSTEVVELWELEPGDIVLVRNGEQI
PVDGNVIAGVGGIDESNITGESMPAEKGQGSDVYAGTWLRSGVLRVEATGIGSDSTLAKI
IHRVEDAQDDKARTQTFLEKFSKWYTPGVMIAAAVVGLITWDVELALTLLVIGCPGALVI
SIPVSIVAGIGRAARDGVLIKGGEYLETAAKVDVVVVDKTGTLTTGRPELTDVEVIEPAY
SQGEVLELAARAETASEHPLADAIIRGAQDRGLSTTLVEAAENITGRGIIANVDGQAVAV
GSAELLDHEPDSTRILELNAEGKTAMFVGVNGHAIGIVAVADAVRSDSASAIESLHKAGI
QVVMATGDAHRVAQNVASKLGVDEVYSELLPEQKLELVRDLQAAGKTVAMVGDGVNDTPA
LAAADIGVAMGVAGSPAAIETADIALMADRLPRLAHAVTLAKRTVRTMRINILIALATVM
VLLAGVLFGGVTMSVGMLVHEASVLLVISIAMLLLRPTLKEDAAQASDIKRSEIQQIA
>RXN01000 TRANSLATE of: rxn01000.seq check: 4854 from: 1 to: 846
MSTLTSHRTVPAPSSPPARPNKLARNIVAIVAALIVLIATGTLKIEWNELPQMPAQVWHY
LELMFSDPDWSKFGRAVQEMWRSIAMAWLGAILCVVVSVPLGMLAARGVGPYWLRTVLRF
VFAVIRAFPEVVIAIILLTVTGLTPFTGALALGISGIGQQAKWTYEAIESTPTGPSEAVR
AAGGTTPEVLRWALWPQVAPSIASFALYRFEINIRTSAVLGIVGAGGIGSMLANYTNYRQ
WDTVGMLLIVVVVATMIVDLISGTIRRRIMKGASDRVVAPSN
>RXN01002 TRANSLATE of: rxn01002.seq check: 1757 from: 1 to: 804
MNSDASATTNSWATNPDHVSVTYPNGTKALDDVSLTINPGEMVAIVGLSGSGKSTLIRTI
NGLVRATEGTVTVGPHQINTLKGKALRDARGQIGMIFQGFNLSERSSVFQNVLVGRFAHT
AWWRNLLGFPTEHDKQIAFHALESVGILHKVWTRAGALSGGQKQRVAIARALSQDPSVML
ADEPVASLDPPTAHSVMRDLENINNVEGLTVLVNLHLIDLARQYTTRLVGLRAGKLVYDG
PTSEATDKDFEATYGRPIQAKDLLGDRA
>RXN01141 TRANSLATE of: rxn01141.seq check: 9956 from: 1 to: 825
LSTALAGAARYVTSTSNNEPADNTPLTIGYVPIAGSAPIAIADALGLFKKHGVNVTLKKY
SGWSDLWTAYATEQLDVAHMLSPMTVAINAGVTNASRPTELSFTQNTNGQAITLASKHYG
SVNSAADLKGMVLGIPEEYSVHALLLRDYLVSNAVDPIADLELRLLRPADMVAQLTVEGI
DGFIGPGPFNERAISNGSGRIWLLTKQLWDKHPCCAVAMAKEWKAEHPTAAQGVLNALEE
ASATLSNPAQFDSSARTLSQEKYLNQPATLLDGPS
>RXN01142 TRANSLATE of: rxn01142.seq check: 3960 from: 1 to: 498
LTARGNIDFGLRSARPSLSKTERADITRTHLEQVGLTDAAERRPARLSGGMQQRVGIARA
FAIDPPIMLLDEPFGALDALTRRELQLQLLNIWEASRRTVVMVTHDVDEAILLSDRVLVM
SKSPEATIITDTPVNLPRPRHELSEDASVEAETTALRKRMLHLLEH
>RXN01164 TRANSLATE of: rxn01164.seq check: 868 from: 1 to: 1635
VTLFVRLALAAVGGLFVFASNEPIGWFVAGIVGTALFFISLAPWDLGVPQKRRKKNEPVP
FLQQMSTGPTVVQGMLLGFVHGLVTYLQLLPWIGEFVGSLPYVALSVVEALYSIALGAFG
VLIARWRDWKVLLFPAMYVAVEYLRSSWPFDGFAWVRLAWGQINGPLANLAALGGVAFVT
FSTVLAAVGVAMVIISKKRLAGAIITASVIAIGAVSSLYVDRNGTSDESIEVAAIQGNVP
RMGLDFNAQRRAVLANHARETLKLDEQVDLVIWPENSSDVNPFSDAQARAIIDGAVEHVQ
APILVGTITVDEVGPRNTMQVFDPVEGAAEYHNKKFLQPFGEYMPFREFLRIFSPYVDSA
GNFQPGDGTGVVEMNAANLGRAVTVGVMTCYEVIEDRAGRDAIANGAEFLTTPTNNATFG
FTDMTYQQLAMSRMRAIEFDRAVVVAATSGVSAIVNPDGSISQNTRIFEAATLTESIPLK
DTVTIAARVGEYVELLLVIIGVLAGLFAIRMNSRSKSAKGSARPAQVRVKKVPAKKAATN
RRKVK
>RXN01168 TRANSLATE of: rxn01168.seq check: 6703 from: 1 to: 810
MSSEAVDATTLVIIPTYNELENLPLIVDRVRTATPDVHVLIVDDNSPDGTGERADKLAAD
DDHIEVLHREGKGGLCAEYMAGFQWGLERDYQVLCEMDADGSHAPEQLHLLLAEITNGAD
LVIGSRYVPGGRVVNWPKNRWLLSKGGNVYISVALGAGLTDMTAGYRAFRREVLEALPLD
ELSNAGYIFQVEIAYRAVEAGFDVREVPITFTEREIGESKLDGSFVKDSLLEVTKWGLKH
RGGQAKELSKEMVGLLNYEWKHFKKRNTWL
>RXN01285 TRANSLATE of: rxn01285.seq check: 1049 from: 1 to: 726
LNVTIPDNTFTAIIGPNGCGKSTLLRGESRVLNPQHGKVLLDGRQLDSFKPKEIARELGL
LPQTSIAPEGIRVYDLIARGRAPYQSLIQQWRTSDEDAVAQALASTNLTELAARLVDELS
GGQRQRVWVAMLLAQQTPIMLLDEPTTFLDIAHQYELLELLRAFNEAGKTVVTVLHDLNQ
AARYADHLIVMKDGHVHATGTPEEVLTAEMVQGVFGLPCIISPDPVTGTPTVVPLSRSRA
GA
>RXN01298 TRANSLATE of: rxn01298.seq check: 8940 from: 1 to: 930
VSTLISEPEVDKLRKRAKRSRRTEWWLAAALLAPNLLLLAIFTYRPLLDNFRLSFFNWNI
SSPTSTFIGFDNYVEEFTRSDTLQVVLNTVIFTACAVIGSMVLGLLLAMLLDQKLFGRNF
VRSMVFAPEVISGAAIGVAFQFVFDPNFGLVQDLLGRIGVDSPQEYQNPNWALFMVTFTF
VWKNLGYSPVIYLAALQGLNKDLSEAAPVDGASAWTRFWKVTLPQLRPTTFFLSITVTLN
SVQVFDIIHTMTRGGPLGNGTTTLVYQVYTETFTNYRAGYGATIATILFLLLLIITVIQV
RYMDKENKQK
>RXN01323 TRANSLATE of: rxn01323.seq check: 658 from: 1 to: 2265
MAQTPAKIPAALNFIDVDLGVTGMTCTSCSARVERKLNKLDGVEATVNYATESAQVSYDP
SKVSPEQLIKTVEDTGYGAFTMASAAAESEEDNAPADSGQSRIDAARDHEAADLKHRVIV
SALLSVPVVLVSMIPALQFNNWQWAVLTLVTPIFFWGGSPFHKATWANLKRGSFTMNTLV
SLGTSAADLWSLWALFIENAGHPGMKMEMHLLPSASTMDEIYLETVAVVITELLLGRWFE
TKAKGQSSEALRKLLDMGAKDAVVLRDGAEVRVPVNQLKLGDVFITRPGEKIATDGEVDE
GSSAVDESMLTGESIPVEVTKGSKVTGATLNTSGRLMVKVTRIGADTTLSQMAKLVTDAQ
SKKAPVQRLVDQISQVFVPVVIVIAIATLIAHLVFTDAGLAPAFTAAVAVLIIAGPCALG
LATPTALLVGTGRGAQLGLLIKGPEILESTKKVDTIVLDKTGTVTTGTMSVTDVTAINYS
ETEILEFAAAVESASEHPIAQAIAKAAEHEQVTDFQNTAGQEVTGVVRGHEVRVGRPSST
LIDALLHPFQHAQKIGGTPVVVTIDGVDSGIITVRDTVKDTSAEAIRGLKELGLTPILLT
GDNEGAAKSVAAEVGIDQVIANVLPHEKVQNVEALQAQGKNVAMVGDGVNDAAALAQADL
GLAMGAGTDVAIEASDITLMNNDLRSAVDAIRLSRKTLGTTKGNLFWAFAYNVALIPVAA
IGLLNPMLAGIAMAFSSVFVVSNSLRLRGFKARSN
>RXN01338 TRANSLATE of: rxn01338.seq check: 9102 from: 1 to: 1902
KTYTPNPWMLFIRSFDGIITVAALVAIAIHLILWLALDLDGLAKNWPLIAIVIVGGIPLM
WDVLKSAIKTRGGADTLAAVSIITSVLLGEWLVAAIIVLMLSGGEALEEAASRRASGTLD
ALARRAPSTAHRLLGATILDGTEEIAVEEITVGDLVAVLPHELCPVDGEIVAGHGTMDES
YLTGEPYVVSKSKGSQAMSGAVNGDTPLTIVATKLAHDSRYAQIVGVLHEAENNRPEMRR
MADRLGAWYTVIALALGGLGWIVSGDPVRFLAVVVVATPCPLLIAVPVAIIGAISLAARR
GIIVKNPGMLENASGVKTVMFDKTGTLTYGRPVITDIHTAPGVEEDTVLALAASVERYSR
HPLADAIREGAKARELHLPDVVEVSERPGQGLTGTVGEHLVRITNRRSTLEIDPDSKNYI
PVTSSGMESVVLVDDKYAALIRLRDEPRASASEFIAHLPKKHKVDKLMIISGDRASEVRY
LADKVGIDEVHAEASPEDKLNIVNRHNEHGATMFLGDGINDAPAMAVATVGVAMGADSDV
TSEAADAVILDSSLERLDDLLHISARMRRIALQSAGGGMALSVIGMILAVFGFLTPLMGA
IFQEVIDVLAILNSARVALPRGAISDFDTQEKVS
>RXN01411 TRANSLATE of: rxn01411.seq check: 3735 from: 1 to: 765
MLGVGWRIPFLMAVPLGLIGWWIRTGAQENVRPASERPEAPIKQALRTEWKMMLRVGGFI
SCTGLSFYIFTTYMTTFLRSTVGLEGTLVLAGNIIALSMAAIVAPFVGRAIDKFPRRNIM
AFATLSTVIMAIPAYIIAGQGTLTASLIAQVMLGIGAVTANCVTSVMMAEVFQEVTRGTS
AGITYNVTYAIFGGSAPFISTALVSWTGSPLAPAVYMIIIALFAFTASRFIPETSPVFVT
ATPATKAPKVLVNPG
>RXN01808 TRANSLATE of: rxn01808.seq check: 4151 from: 1 to: 1149
QSLACKELAWMRGGAPARTSKPGFRLEAAEALIAEVPAPRDKVELMAFSKSRQGRVVIEL
EDATVATPDDRILVEDLTWRLAPGERIGLVGVNGSGKTTLLRTLAGEQPLQAGKRIEGQT
VKLGWLRQELDDLDLSRRLIDCVEDVASYVMMGDKQVSASQLAERLGESPKRQRTPVGDL
SGGERRRLQLTRVLMAEPNVLLLDEPTNDLDIDTLQELESLLDGWPGTMVVISHDRYLIE
RVTDSTWALFGDGKLTNLPGGIEEYLQRRAAMAAAEDSGVLNLGAATQAGTFSAATEQAA
TSVESSGISSQERHRITKEMNALERKMGKLDQQMDKLNQQLADAAEAMDTIKLTELDTKL
RAVQEEHGELEMQWLELGEEIEG
>RXN01939 TRANSLATE of: rxn01939.seq check: 574 from: 1 to: 1731
MTTNIPQTPNHEGEQPLLELKDLKISFTSSTGVVDAVRGANLTIYPGQSVAIVGESGSGK
STTAMSIIGLLPGTGKVTEGSIMFDGQDITGLSNKQMEKYRGSEIGLVPQDPMTNLNPVW
RIGTQVKESLRANHVVPGSEMDKRVAEVLAEAGLPDAERRAKQYPHEFSGGMRQRALIAI
GLAARPKLLIADEPTSALDVTVQRQILDHLETLTKDLGTAVLFITHDLGLAAERAEHLVV
MHRGRIVESGPSLKILRNPQHPYTQRLVKAAPSLASARIQSAQEQGIESAELLSATAVAE
GTIPEMEEKVIEVKNLTREFDIRGARGDKKKLKAVDDVSFFVRKGTTTALVGESGSGKST
VANMVLNLLEPTSGEVLYNGTDLTSLSHKEIFQMRRKLQVVFQNPYGSLDPMYSIYRCIE
EPLTIHKVGGDRKAREARVAELLDMVSMPRSTMRRYPNELSGGQRQRIAIARALALNPEV
IVLDEAVSALDVLVQNQILTLLAELQQELKLTYLFITHDLAVVRQTADDVVVMQKGRIVE
KGRTDDIFNDPQQHYTRDLINAVPGLGIELGTGENLV
>RXN01995 TRANSLATE of: rxn01995.seq check: 3763 from: 1 to: 1338
MDIRQTINDTAMSRYQWFIVFIAVLLNALDGFDVLAMSFTANAVTEEFGLSGSQLGVLLS
SALFGMTAGSLLFGPIGDRFGRKNALMIALLFNVVGLVLSATAQSAGQLGVWRLITGIGI
GGILACITVVISEFSNNKNRGMAMSIYAAGYGIGASLGGFGAAQLIPTFGWRSVFAAGAI
ATGIATIATFFFLPESVDWLSTRRPAGARDKINYIARRLGKVGTFELPGEQSLSTKKAGL
QSYAVLVNKENRGTSIKLWVAFGIVMFGFYFANTWTPKLLVETGMSEQQGIIGGLMLSMG
GAFGSLLYGFLTTKFSSRNTLMTFMVLSGLTLILFISSTSVPSIAFASGVVVGMLINGCV
AGLYTLSPQLYSAEVRTTGVGAAIGMGRVGAISAPLLVGGLLDSGWSPTQLYVGVAVIVI
AGATALIGMRTQAVAVEKQPEALATK
>RXN02062 TRANSLATE of: rxn02062.seq check: 5414 from: 1 to: 1170
MRVGMMTREYPPEVYGGAGVHVTELTRFMREIAEVDVHCMGAPRDMEGVFVHGVDPALES
ANPAIKTLSTGLRMAEAANNVDVVHSHTWYAGLGGHLAARLHGIPHVATAHSLEPDRPWK
REQLGGGYDVSSWSEKNAMEYADAVIAVSARMKDSILAAYPRIEPDNVRVVLNGIDTELW
QPRPTFDDAEDSVLRSLGVDPQRPIVAFVGRITRQKGVEHLIKAAALFDESVQLVLCAGA
PDTPEIAARTTALVEELQAKREGIFWVQDMLGKDKIQEILTAADTFVCPSIYEPLGIVNL
EAMACNTAVVASDVGGIPEVVVDGTTGALVHYDENDVETFERDIAEAVNKMVADRETAAK
FGLAGRERAINDFSWATIAQQTIDVYKSLM
>RXN02096 TRANSLATE of: rxn02096.seq check: 3261 from: 1 to: 1692
MGLDVSDEQIEHAARLAQAHDFIDRLPNKYEEVIGERGLTLSGGQRQRIALARAFLAHPK
VLVLDDATSAIDASTEDRIFQALREELHDVTILIIAHRHSTLELGDRVGLVEDGRVTALG
PLSEMRDHARFSHLMALDFQDSHDPEFTLDNGSLPSQEQLWPEVSTEKQYKILAPAPGRG
RGMSMPATPELLAQIEALPAATEETRVDAGRLRTSTSGFKLLSLFKQVRWLVVAVIALLL
VGVAADLAFPTLMRAAIDNGVQAQSTSTLWWIAIAGSVVVLLSWAAAAINTIITARTGER
LLYGLRLRSFVHLLRLSMSYFERTMSGRIMTRMTTDIDNLSSELQSGLAQTVVSVGTLIG
VVTMLAITDAQLALVALSVVPIIIVLTLIFRRISSRLYTASREQASQVNAVFHESIAGLR
TAQMHRMEDQVFDNYAGEAEEFRRLRVKSQTAIAIYFPGLGALSEIAQALVLGFGALQVT
RGDISTGVLVAFVLYMGLMFGPIQQLSQIFDSYQQAAVGFRRITELLATQPSVQIWAPTG
TLGRLPRSLYCLTTSPSAIQTIRS
>RXN02348 TRANSLATE of: rxn02348.seq check: 8038 from: 1 to: 1884
MLNRMKSARPKSVAPKSGQALLTLGALGVVFGDIGTSPLYSLHTAFSMQHNKVEVTQENV
YGIISMVLWTITLIVTVKYVMLVTRADNQGQGGILALVALLKNRGHWGKFVAVAGMLGAA
LFYGDVVITPAISVLSATEGLTVISPSFERFILPVSLAVLIAIFAIQPLGTEKVGKAFGP
IMLLWFVTLAGLGIPQIIGHPEILQSLSPHWALRLIVAEPFQAFVLLGAVVLTVTGAEAL
YADMGHFGARPIRVAWFCVVMPALILTYLGQGALVINQPEAVRNPMFYLAPEGLRIPLVI
LATIATVIASQAVISGAYSLTKQAVNLKLLPRMVIRHTSRKEEGQIYMPLVNGLLFVSVM
VVVLVFRSSESLASAYGLAVTGTLVLVSVLYLIYVHTTWWKTALFIVLIGIPEVLLFASN
TTKIHDGGWLPLLIAAVLIVVMRTWEWGSDRVNQERAELELPMDKFLEKLDQPHNIGLRK
VAEVAVFPHGTSDTVPLSLVRCVKDLKLLYREIVIVRIVQEHVPHVPPEERAEMEVLHHA
PIRVVRVDLHLGYFDEQNLPEHLHAIDPTWDNATYFLSALTLRSRLPGKIAGWRDRLYLS
MERNQASRTESFKLQPSKTITVGTELHL
>RXN02354 TRANSLATE of: rxn02354.seq check: 8723 from: 1 to: 834
MTKRTKGLILNYAGVVFILFWGLAPFYWMVITALRDSKHTFDTTPWPTHVTLDNFRDALA
TDKGNNFLAAIGNSLVISVTTTAIAVLVGVFTAYALARLEFPGKGIVTGIILAASMFPGI
ALVTPLFQLFGDLNWIGTYQALIIPNISFALPLTIYTLVSFFRQLPWELEESARVDGATR
GQAFRMILLPLAAPALFTTAILAFIATWNEFMLARQLSNTSTEPVTVAIARFTGPSSFEY
PYASVMAAGALVTIPLIIMVLIFQRRTVSGLTAGGVKA
>RXN02356 TRANSLATE of: rxn02356.seq check: 7192 from: 1 to: 996
MATVTFDKVTTRYPGAERATVHELDLDIADGEFLVLVGPSGCGKSTTLRALAGLEGVESG
VIKIDGKDVTGQEPADRDIAMVFQNYALYPHMTVAKNMGFALKLAKLPQAQIDAKVNEAA
EILGLTEFLDRKPKDLSGGQRQRVAMGRALVRDPKVFLMDEPLSNLDAKLRVQTRAEVAA
LQRRLGTTTVYVTHDQVEAMTMGDRVAVLKDGLLQQVAPPRELYDAPVNEFVAGFIGSPS
MNLFPANGHKMGVRPEKMLVNETPEGFTSIDAVVDIVEELGSESYVYATWEGHRLVARWV
EGPVPAPGTPVTFSYDAAQAHHFDLESGERIA
>RXN02391 TRANSLATE of: rxn02391.seq check: 7541 from: 1 to: 399
MTQSDLPDDVQELVTKIFGLARDGGAESAATLGAYVDNGVDVNLSNQDGNTLLMLAAYAG
HADVVQALIERGADVDRVNNRNQTPLAGAIFKKEEAVIEALLAGGADPYAGTPTAVDTAK
MEGREDLVARFES
>RXN02442 TRANSLATE of: rxn02442.seq check: 5164 from: 1 to: 849
MKFFTDALIVPFDVSFISRALVAGCLAAILCSLIGTWVILRRLTFFGDAMSHGLLPGVAT
ASLLGGNLMFGAAISALIMSAGVVWTSRKSSLSQDVSTGLQFITMLSLGVVIVSHSDSHA
VDLTSFLFGDILGVRPSDIFIIAIATVLGGLTIFLFHRQFTALAFDERKAHTLGLNPRFA
HLLMLALIALATVVSFQVVGTLLVFGLLIGPPATAALLVQDKASISLIMIVASLLGCAEI
YLGLLISWHASTAAGATITLLSAAIFFATLLTKSAISRLNFTA
>RXN02447 TRANSLATE of: rxn02447.seq check: 8454 from: 1 to: 1095
TVVPVYLAELAPLEIRGSLTGRNELAIVTGQLLAFVINALIAVTLHGVIDGIWRIMFAVG
ALPAVALFLGMLRMPESPRWLVNQGRYDDARRVMETVRTPERAKAEMDEIIAVHSENNAA
LPGVKQSSGQASGQVSSKHTHMSIGEVLSNKWLVRLLIAGIGVAVAQQLTGINAIMYYGT
RVLEESGMSAEMAVVANIAFGAVAVTGGLIALRNMDRLDRRTTFIIGLSLTTTFHLLIAA
AGTLLPEGNSIRPFAIMILVVGFVLSMQTFLNVAVWVWLAEIFPVRMKGIGTGISVFCGW
GINGVLALFFPALVSGVGITFSFLIFAVVGVIALAFVTKFVPETRGRSLEELDHAAETGQ
IFRKA
>RXN02455 TRANSLATE of: rxn02455.seq check: 2559 from: 1 to: 1269
LKRLTRIASISMASMLAAASLVACSGSTDEEGDVYFLNFKPEQDVAYQEIAKAYTEETGV
KVKVVTAASGSYEQTLKAEIGKDEAPTLFQVNGPAGFITWQDYMADMSDTEVAKQLTDDI
PPLTTEDGEVRGVPFAVEGFGTTYNDEIFDKYIATSGAKIKSTDEITSYQKLKEVAEDMQ
AKKDELGIEGAFASTSLTSSEDWRWQTHLANAPIWQEYQDKGVEDTNEIEFSYNKEYKNL
FDLYLENSTVEKSLAPSKTVSDSMAEFAQGKAANVQNGNWAWSQISETSGNVVKEDKIKF
LPMYMGLPDEEKHGINVGTENYLGVNSEASEVDQQATKDFVDWLFTSEAGKEHVVKDLGF
IAPFESYTAENTPNDPLSEQVAEAIANKDLTTYPWNFQYFPSQQFKDDFGQDLSQYASGK
LKW
>RXN02515 TRANSLATE of: rxn02515.seq check: 4857 from: 1 to: 756
MSTLEIRNLHAQVLPSDESAEPKEILKGVNLTINSGEIHAIMGPNGSGKSTLAYTLGGHP
RYEVTAGEVLLDGENILEMEVDERARAGLFLAMQYPTEIPGVSVANFLRSAATAIRGEAP
KLREWVKEVRTAQEALAIDPEFSNRSVNEGFSGGEKKRHEVLQLDLLKPKFAIMDETDSG
LDVDALRIVSEGINSYKQETEGGILMITHYKRILNYVKPDFIHVFANGQIVTTGGAELAD
KLEADGYDQFIK
>RXN02549 TRANSLATE of: rxn02549.seq check: 8075 from: 1 to: 2703
MVHAKQTKKPLPRFLHSAHFYVWIVLGFVVFAQPYGQVAADTKLDLLLNPAGFLTGALHA
WTDTFTLGQLQNQAYGYLFPQGFFFLITDFLPDWIAQRLWWWLVLGLGFSGFYALVARLG
IGNPAFRVIAALLFALSPRTLTTLTAISSETWPIMLAPWVCLPLLSRNVDARAIALSLLP
AACMGAVNATATMAALIPAALILLYRGLFLRLLLWGMGVLAVNSWWIGPLLVLGKYAPPF
TEFIESSSVTTSWLNPVEILRGTTSWTPFVDTERQAGYLLVNDALFVTLSVLVAALGLIG
LTLMKHRGLWAFMLAIGLLILGSAHLTAVQEFLDGPGAALRNIHKFDLLVRNPLMVGVAA
LGSHISLPLLGTTALTSGQGKHHTIPLPLQKRQAAGLLVVIIAVGALAPAWSARLLPQGT
WDEVPDYWYEATEFLNQNATGTRTLIWPSSPFARQDWGWTRDEPAQPLLDVPWAVRDAIP
LVPPEAIRGLDGLDDLGTLGTGLNDEALKRLGIGAVLVRHDLEADPDIEVDLPGEKHTFG
SQGQVDVYLTDPDRNMWITSGTSKQLPTVAGGGEILSLLDTINGYSPRTLVSENAQIVTD
TPQLVGTNYGDGTSSAALASLDETEVKNRIVDYPSAGPMTQVVQEGSITASSSGSDATSF
GGADPDRSLNSLLDHRYNTAWYPTPGDTSPWLEVSGTGTTLSISPRSTVTATITSGDSVM
VREFEKGRTTTVTLAEPEARIEFDGFVGISELSLEGLSRTITVPETSPDVQQFVPQRLTV
PTSFLDRTFTVPRHMSVTVEAQSCVTLELDGDRIDCGPSNSPPEPTRCAPNRNGSPSPNP
LRSPLFSQQQTSRQHPPTACSSPRALSIQVPARLSTPPPFPQSNSTPPPKVSSSPRTPPA
S
>RXN02570 TRANSLATE of: rxn02570.seq check: 2673 from: 1 to: 642
MNPLTWITGAFSMWTVVLGVNKLGLSIAVIIIAQVVAMTRVRNVSVLASTALLSVPALAS
MALIHMPYSSDGWLIALTLTARFSALMSIFLLAATAITIPELVKSLYRWPKLAYIVGSAL
QMIPQGKQTLALVRDANALRGRSVKGPVRAVKYVGLPLITHLLSAGAARAIPLEVAGLDR
PGPRTVLVEVVEGRVEKHCRWLLPLLAVGMAWWL
>RXN02595 TRANSLATE of: rxn02595.seq check: 5016 from: 1 to: 1164
VIVVAMASIMACLKAARLNNPMKILLLCWRDTTHPQGGGSERYLERVGEFLADQGHEVVF
RTAGHTDAPRRSFRDGVRYSRSGGKFSVYPKAWVAMMLGRVGIGTFSKVDVVVDTQNGIP
FFGKFFSGKPTVLLTHHCHKEQWPVVGRVLAKVGWLIESQIAPRAYKTAPYVTVSEPSAE
ELIALGVDQQRIHIVRNGVDPVPLHTPKLDRDGQHAVTLSRLVPHKQIEHAMDVVAALDG
VVLDVVESGWWQKELVDYARTLGVSDRVVFHGQVAEDHKHALLERATIHLMPSRKEGWGL
AVTEAAQHGVPTIGYRSSGGLRDSVVDGETGLLVDSKAELISATKTLLIDASLRSKLGAS
AKQRAENYKWDTAGAQFEELLLGLASKK
>RXN02614 TRANSLATE of: rxn02614.seq check: 5216 from: 1 to: 729
MTATLSLKPAATVRGLRKSYGTKEVLQGIDLTINCGEVTALIGRSGSGKSTILRVLAGLS
KEHSGSVEISGNPAVAFQEPRLLPWKTVLDNVTFGLNRTDISWSEAQERASALLAEVKLP
DSDAAWPLTLSGGQAQRVSLARALISEPELLLLDEPFGALDALTRLTAQDLLLKTVNTRN
LGVLLVTHDVSEAIALADHVLLLDDGAITHSLTVDIPGDRRTHPSFASYTAQLLEWLEIT
TPA
>RXN02795 TRANSLATE of: rxn02795.seq check: 7318 from: 1 to: 1437
VLKVSDLTVGNNFVHNVSFEVNPGERVGIIGESGSGKSLTALSIMGLTDLPTTGQITFNG
KPSATFRGTRIANVFQEPMSALNPLMRIGRQIEEMMTLHGASKKDARARLKSLLIDVSLP
ERTASAYPHELSGGQRQRALIAMALANDPDLLICDEPTTALDVVVQKQIVDLLLRLTKER
GTALLFITHDLGLIARTCERLLVMKSGETVERGDTEAILRSPAHSYTQQLLDASILDQPE
IASDSGAPVVIDVEEASKSFKETTALHKVSLAVRKGDLLGIVGGSGSGKTTLLKLIAGLD
KPTTGTVAVTGGVQMVFQDPQSSLNPRMKIKDIVAEPLLGWNAAEKTTRVAEVITQVGLS
PDVLDRYPHEFSGGQRQRISIARALATKPAILLADEPVSALDVSVRKQVLDLLQQLVEEY
GITLVFVSHDLAVVRHLCTTVWVMEQGRVLEQGPIDSVYDHPQTEYTKELLDAVPRLSL
>RXN02925 TRANSLATE of: rxn02925.seq check: 5237 from: 1 to: 2217
MSTPHHHGDHPAPETDHTHHPNHAGHEHHADAATHGQAMPHDRPHSTVDEEHQVHSHGEH
AGHSAANFRDRFWWSLILSVPVVFFSPMPADLLGYNIPEIPGAYWIPPVLGTIIFLYGGT
PFLKGAMTELKSRQPGMMLLIAMAITVAFIASWVTTLGLGGFHLDFWWELALLVTIMLLG
HWLEMRALGAASSALDALAALLPDEAEKVVDGTTRTVAISELAVDDVVLVRAGARVPADG
TIIDGAAEFDEAMITGESRPVYRDTGETVVAGTVATDNTVRIRVEATGGDTALAGIQRMV
ADAQASSSRAQALADRAAALLFWFALITALITAVVWTIIGSPDDAVVRAVTVLIIACPHA
LGLAIPLVIAISSERAAKSGVLIKDRMALEHMRTIDVVLPDKTGTLTEGAHAVTGVAPAT
GIAEGELLALAAAAEADSEHPVARAIVTAAAAHPEASQRQLRATGFTAASGRGIRATVDG
AEILVGGPNMLREENLTTPGELADITGSWAQRGAGVLHVVRDGEIIGAVAVEDKIRPESR
AAVRALQARGVKVAMITGDATQVAQAVGKDLGIDEVFAEVLPQDKDTKVTQLQERGLSVA
MVGDGVNDAPALARAEVGIAIGAGTDVAMESAGVVLASDDPRAVLSMIELSHASYRKMVQ
NLVWATGYNIVAVPLAAGVLAPIGVLLPPAAAAILMSLSTIIVALNAQLLRRIDLDPAHL
APTDGKEEKAAVSSAAPVR
>RXN02933 TRANSLATE of: rxn02933.seq check: 4913 from: 1 to: 810
MPLSGKIGGFIVAVVFVLAALSFIWTPFDPVQAFPQERLEGSSLRHLLGTDRYGRDVLSQ
IMVGSRVTLLVGIIAVAIAALIGTPLGIAAGMRRGMVETFVMRGADLMLAFPALLLAIIS
GAVFGASTWSAMVAIGIAGIPSFARVARAGTLQVTSQDFIAAARLSKVSSARIALRHILP
NITSMLIVQASVAFALAILAEAALSFLGLGTTPPDPSWGRMLQTAQASIGVTPMLAVWPG
AAIALTVLGFNLFGDGLRDAIDPKREVGRA
>RXN02945 TRANSLATE of: rxn02945.seq check: 2147 from: 1 to: 933
MTTALGTRVVARNFGYRHASRENPALKDINFEIAPGERILLTGASGAGKSTLLAALAGVL
GGSDEGVSTGELLVDAPSIGLVLQDPDSQVIASRIGDDVAFGCENLQIPREEIWPRVERA
LELVGLDLPLSHPTKYLSGGQKQRLALAGVIAMGARLILLDEPTANLDPQGQKNVVAAVD
RVVQETGATLIVVEHRHELWVNIIDRIISITDGEDVQPAELIKVGQLPGAQPSTSKPILW
ANDLLCTWGGLRSFEVPEGASTVITGPNGAGKSTLALTMGGLLPRKVGSWNSLTRCAAAL
TRPRTSGVQLI
>RXN02975 TRANSLATE of: rxn02975.seq check: 5313 from: 1 to: 249
VIVTNDLEVRVGARTLLDAPGQLLRVQPGDRIGLVGRNGAGKTTTMRILSGETKPYGGSV
TTSGEIGYLPQDSREGNIEQTAR
>RXN02994 TRANSLATE of: rxn02994.seq check: 4127 from: 1 to: 723
IKMTGVQKYFGDFHALTDIDLEIPRGQVVVVLGPSGSGKSTLCRTINRLETIEEGTIEID
GKVLPEEGKGLANLRADVGMVFQSFNLFPHLTIKDNVTLAPIKVRKMKKSEAEKLAMSLL
ERVGIANQADKYPAQLSGGQQQRVAIARALAMNPKIMLFDEPTSALDPEMVNEVLDVMAS
LAKEGMTMVCVTHEMGFARKAADRVLFMADGLIVEDTEPDSFFTNPKSDRAKDFLGKILA
H
>RXN03020 TRANSLATE of: rxn03020.seq check: 1931 from: 1 to: 603
MTLHVSNLNLTVADGSTSRTLLNNIHFWMSNQAKSSVSPAHPAPENPPYSPSSAASKAPD
SGTATLGDIDLLNPQNRAALRRNHLGIVFQQPNLLPSLTVLDQLLLIPRLGRILPPSRSA
RTQHKDKALSLLNSIGLGDLAKRKVSELSGGQQARVNLARALMNSPKLLLVDEPTAALDQ
HSASEVTELIVSMAHQYNAPT
>RXN03080 TRANSLATE of: rxn03080.seq check: 3725 from: 1 to: 780
MPQLVEIRDLNVEFPSRHAVKNVSFSAPAGKVTALIGPNGAGKSTALSAIAGLVESTGEV
MVGGSGVASKSAKARARLLSLVPQNTELRIGFSARDVVAMGRYPHRGRFAVETDADRRAT
DDALRAINALDIAEQPVNELSGGQQQLIHIGRALAQDTAVVLLDEPVSALDLRHQVEVLQ
LLRARANSGTTVIVVLHDLNHVARWCDHAVLMADGEVVSQGDIREVLEPATLSTVYGLPI
AVRDDPETSSLRVIPHPNPF
>RXN03081 TRANSLATE of: rxn03081.seq check: 3848 from: 1 to: 459
MKKSLIAIVASALVLSGCTSDSSDSSGTSGTVETTSITTSVAAADGAFPRTVTLDDSSIT
LESKPERIAVLTPEAASLVLPITGADRVVMTAEMDTADEETAALASQVEYQVKNGGRLDP
EQVVAGDPDLVIVSARFDTEQGTIDILEGLNVP
>RXN03108 TRANSLATE of: rxn03108.seq check: 138 from: 1 to: 267
MTKPNASVELNTITKSYGSTTIIGDTSITINDGEFVSLLDPSGCGKSTILKMIAGLASPS
TGTVSAGNEEIKGPGPDRGMVFQDHALLP
>RXN03116 TRANSLATE of: rxn03116.seq check: 7423 from: 1 to: 609
MGEGDVEKHFAFGLKAAKQRRFFARTVALMPQNPTIPAGLSVFDYVLLGRHPHSYAPGRA
DDEIVKRCLADLKLEHFSDRGLDELSGGERQRVSLARALAQEPRIVLLDEPTSALDIGHA
QETLELIDAIRHRLGLTVIAAMHDLTLTAQYGDRVLMMNGGRKVFEGTAAEVLTAQRISE
IYDATVIVEVIDGRPVVIPQRSH
>RXN03129 TRANSLATE of: rxn03129.seq check: 210 from: 1 to: 1224
MASIVFENVTRKYSPGARPAVDKLNLEIADGEFLVLVGPSGCGKSTSLRMLAGLEPIDEG
RLLIDGKDATELRPQDRDIAMVFQSYALYPNMTVRDNMGFALKNQKVAKAEIEKRVAEAS
RILQLDPYLDRKPAALSGGQRQRVAMGRAIVREPSVFCMDEPLSNLDAKLRVSTRAEISG
LQRRMGVTTVYVTHDQVEAMTMGDRVAVLLLGVLQQVDTPQNLYDYPANAFVASFIGSPS
MNLIEGTIRGDKVTLGTGIQISVPDEVAAEVRNNPDRFEGRPVIVGARPEHMYLTTANES
GAVLGEVSHIDELGADSMVYVLASGVKNPNTDLLGEGIPEDMRVTVVGAEETDKARLGIR
VERHHGLKAGDKVHVVAAPKDVHLFDGLDGRRIGASVLAPAHTVQSGH
>RXN03164 TRANSLATE of: rxn03164.seq check: 9986 from: 1 to: 870
MIYRRVGNSGLKLPAISLGLWHNFGDDKPLSTQRSIIHRAFDRGVTHFDLANNYGPPAGS
AETNFGRILREDLKSHRDELIISSKAGWDMWPGPYGFGGSRKYLVSSLDQSLTRLGLDYV
DIFYHHRPDPDTPLEETMYALRDIVASGKALYVGISSYGPELTAEAAEFMAEEGCPLLIH
QPSYSIINRWVEEPGDDGENLLQSAANNGLGVIAFSPLAQGLLTDKYLDGIPEGSRASQG
KSLSEGMLNVNNIDMVRKLNDIAQERGQSLAQNALAWVLREQREYGAGLP
>RXS00088 TRANSLATE OF: RXS00088.seq check: 1389 from: 1 to: 876
IEDNHGTEGISLPIEGVAATDNRAFELLDRWGVELVAAPLQLVPFTVTGYTEEGGVANLGSHREPDLEA
LAAAQPSLIINGQRFAQYYDDIIALNPDATVVELDPRDGEPLDQELIRQAETLGEIFGEEEDAAKIVAD
FESALERAKTAYAAISDQTVMAVNVSGGNIGYIAPSVGRTYGPIFDLVGLTPALEVGNASSDHEGDDIN
VEAIAAANPDLILVMDRDGGTSTRNEADYVPAEQIVSDNEALANVKAVTDGYVYYAPADTYTNENIITY
TEILNGMADMFEKAAQ
>RXS00372 TRANSLATE OF: RXS00372.seq check: 2326 from: 1 to: 1077
MSSKHPLKRTAVTVFALGASAALLVACSEPSEDVSTAETTTASSSANASDAAGEKVTITVYTSEPEEKV
DEINKAFMEANPDIEVEVYRAGTGDLTARIEAEKASGSIEADVLWAADAATFETYAAQGDLAELEDVET
SDIIEEALDAENFYVGTRIIPTVIAYNTEVVDQAELPTSWADLTDPKYAGQLVMPDPAVSGAAAFNASV
WKNDPALGEAWITALGENQPMIAQSNGPTSQEIAGGGHPVGIVVDYLVRDLAAAGSPIDTIYASEGSPY
ITEPAGVFADSEKKEAAERYINFLLSVEGQEIAVEQAYLPVREDVGTPEGTPELADIELMTPDLEVVTA
DKAAAVEFFQNAMN
>RXS00453 TRANSLATE OF: RXS00453.seq check: 3260 from: 1 to: 2349
VISAWLLILAIVGGLALTMQKGFSNSFTIEDTPSIDATVSLVENFPDQTNPVTAAGVNVVFQSPEGTTL
DDPQMMTAMDAVVDYIEDNLPDFGGGERFGNPVEVSPALEEMVIEQMTSMGLPEETAAKDAANLAVLSE
DKTIGYTSFNIDVEAAEYVEQKHRDVINEAMQIGEDLGVRVEAGGPAFGDPIQIETTSEIIGIGIAFIV
LIFTFGSLIAAGLPLITAVIGVGIGALAIVLATAFTDLNNVTPVLAVMIGLAVGIDYALFILSRYRAEY
KRMPRADAAGMAVGTAGSAVVFAGATVIIALVALIIADIGFLTAMGISAAFTVFVAVLIALTFIPALLG
VFGGHAFKGKIPGIGGNPTPKQTWEQALNRRSKGRSWVKLVQKAPGLVVAVVVLGLGALTIPAMNLQLS
LPSDSTSNIDTTQRQSADLMAEGFGAGVNAPFLVIVDTHEVNADSTALQPLIEAQEPEEGEFDREQAAR
FATYMYVTQTYNSNIDVKNAQIISVNDDFTAAQILVTPYTGPADKETPELMHVLRAQEAQIEDVTGTEL
GTTGFTAVQLDITEQLEDAMPVYLAVVVGLAIFLLILVFRSLLVPLVAGLGFLLSVGAAFGATVLVWQE
GFGGFVNTPGPLISFMPIFLIGVTFGLAMDYQVFLVTRMREHYTHHNGKGQPGSKYTPVEQSVIEGFTQ
GSRVVTAAALIMIAVFVAFIDQPLPFIKIFGFALGAGVFFDAFFIRMGLVPASMFLMGKATWWMPKWLD
RILPSLDIEGTALEKEWEEKQAAR
>RXS00479 TRANSLATE OF: RXS00479.seq check: 9191 from: 1 to: 2190
MSTSITTENKKKSGPPRLMRIFLPALLILVWLVGAGVGGPYFGKVSEVSSNSQTTYLPESADATQVQEQ
LGDFTDSESIPAIVVMVSDEPLTQQDITQLNEVVAGLSELDIVSDEVSPAIPSEDGRAVQVFVPLNPSA
ELTESVEKLSETLTQQTPDYVSTYVTGPAGFTADLSAAFAGIDGLLLAVALAAVLVILVIVYRSFILPI
AVLATSLFALTVALLVVWWLAKWDILLLSGQTQGILFILVIGAATDYSLLYVARFREELRVQQDKGIAT
GKAIRASVEPILASGSTVIAGLLCLLFSDLKSNSTLGPVASVGIIFAMLSALTLLPALLFVFGRVAFWP
KRPKYEPEKARAKNDIPASGIWSKVADLVEQHPRAIWVSTLIVLLLGAAFVPTLKADGVSQSDLVLGSS
EARDGQQALGEHFPGGSGSPAYIIVDETQAAQAADVVLNNDNFETVTVTSADSPSGSAPITADGIVPLG
SGTAPGPVVVEGQVLLQATLVEAPDSEEAQKAIRSIRQTFADENISAVVGGVTATSVDTNDASIHDRNL
IIPIVLLVILVILMLLLRSIVAPLLLVVTTVVSFATALGVAALLFNHVFSFPGADPAVPLYGFVFLVAL
GIDYNIFLVTRIREETKTHGTRLGILRGLTVTGGVITSAGVVLAATFAALYVIPILFLAQIAFIVAFGV
LIDTLLVRAFLVPALFYDIGPKIWWPSKLSNQKYQKQPQL
>RXS00654 TRANSLATE OF: RXS00654.seq check: 6625 from: 1 to: 1266
VLDILIYPVSGVMKLWHLLLHNVAGLDDSLAWFFSLFGLVITIRAIIAPFTWQMYKSGRTAAHIRPHRA
ALREEYKGKYDEASIRELQKRQNDLNKEYGINPLAGCVPGLIQIPIVLGLYWALLRMARPEGGLENPVF
QSIGFLTPEEVESFLAGRVSNVPLPAYVSMPTEQLKYLSTTQAEVLSFVLPLFITAAILTAINMAMSMY
RSFQTNDYASGFSNGMLKFMIVMSILAPIFPLSLGLTGPFPTAIALYWVSNNLWTLLQTIIMMVILERK
YPLTDDFKVHHLEQRDIYRAKQKEKRIFLWTRRKNRALMILTPWNASTLHATNVELTKTRTAEINEAKQ
ARKEIANKRRETQREMNRAAMQRLKQRRAEVKAKKKGLIDASPNEDTPSENEETKLSSPQVEPTTTAEP
NREPSQED
>RXS00758 TRANSLATE OF: RXS00758.seq check: 161 from: 1 to: 1602
MTLKKSLAVTTAAALALSLAACSSDSSSDSSSSSSGSEGGDNYVLVNGTEPQNPLVPGNTNEVGGGRIV
DSIFSGLVYYDVDGSPVNDVAESIELEGDKTYRITIKDGQTFTDGTPVTAESFVNAWNYNVANSTLSSY
FFESILGYEEGVESMEGLQVVDDTTFTVELTQPESDFPLRLGYSAFFPLPESAFDDMDAFGENPIGNGP
YKLQEWNHNQDATIVPNADYTGGRQAQNDGVKFIFYPTFDSAYADLLSDNLDVLDAIPDSAFSSFEDEL
SGRSINQPSAVFQSFTIPESLEHFSGEEGVLRRQAISLAVNRDEITQTIFEGTRTPATDFTSPVIDGHS
DSLQGADVLTYDPERAQELWAQADEISPWSGEFSISYNADGGHQAWVDATANSIRNTLGIDAIGNPYPD
FKSLRDDVTNRTINGAFRTGWQADYPSLGNFLGPLYGTGAGSNDGDYSNPDFDAKLAEAANAADVDAST
PLYNEAQEILLQDLPAIPTWYSNAVGGYSTNVDNVEFQWNSQPAYYQITKN
>RXS00912 TRANSLATE OF: RXS00912.seq check: 8141 from: 1 to: 273
MDNTLYTAGLTIAAAFFMLSFIFTIYRIIVGPNSIDRLLGLDGTVSMIQCSMATYICWTLDTTVTNFMM
VIALLGFISSVSVARFRKRDGA
>RXS00932 TRANSLATE OF: RXS00932.seq check: 6704 from: 1 to: 474
MTPQKLHRFAALLEMGTWTLLIIGMILKYSGVTDAVTPIAGGIHGFGFLCFAAITITVWINNKWTFPQG
IAGLIVSVIPWAALPFALWADKKGLVAGGWRFSDPSEKPHTFFDKILAQLVRHPIRSILILLVIIAVVF
STLLAMGPPYDPDAIANTVD
>RXS01346 TRANSLATE OF: RXS01346.seq check: 3214 from: 1 to: 1575
MRTATKVIATVMASTLAIGLASCSSSSGTPDVNYVSVNGTEPQRGLIPGDTNENGGGRVVDMLYSGLVY
FDEAGVAQNDLAASIDQETDTTYKITLRDGIKFSDGSDITATDFVDTWNFVVENGLLNTSFFSPIKGYE
EGVETLEGLNVVDDRTFTIELAQPDSEFTQRIGYYGFAPMPASARDDIDAFGENPVSSGPYKLEQWDHN
AELKVVANEHYDGPRAANNDGLKYVFYAQNDAAYSDLLAGNLDVLDLIPPSAYTTYEEELSGRSINQPA
ASYLELSIRMESPNFEGQQGQLRRQAISMAINREEIAEQIFAGTYTPALDFTAPVLDGWRDDLNGNDVL
TFQPDKARELWEDAEEIAPFEGELQISYNADVPNREWVDAVANSISNELDVNATGNPFPDFKSFRDTYR
TTGLDGAYRTAWFADYPSIGNFLGPNYTSGVASNDAKYENPEFDQLIADAAAASTKEETFQAYAQAQEM
LLRDLPAIPLWYPNVVGGYSESVDNVSVNWKAIPVYWAITKQ
>RXS01425 TRANSLATE OF: RXS01425.seq check: 9957 from: 1 to: 885
VLSPDSGITWALSIMFLTFTVRMVLVKPMVNTMRSQRKMQDMAPKMQAIREKYKNDQQKMMEETRKLQK
EVGVNPIAGCLPMLVQIPVFLGLFHVLRSFNRTGSGVGQLEMTVEQNANTPNYIFGVDEVQSFLRADLF
GAPLSSYITMPADAFDAFLGLDVSRLNIALVAAPMILIIVVATHMNARLSVNRQEARKAAGKQQAASSD
QMAMQMQMMNKMMLWFMPATILFTGFIWTIGLLVYMMSNNVWTFFQQRYIFAKMDAEEAAEEEEKRAAK
RTTAPKPGVKPENPKKRKK
>RXS01658 TRANSLATE OF: RXS01658.seq check: 7999 from: 1 to: 1833
DPQILSPTFTQQQQLRNFYGFPDQLAMDRFEVDGKLRDFVVAARELDPNALQQNQQDWINRHTVYTHGN
GFIAAQANQVDEVARDVGSTRGGYPVYTVSDLQSNARAAESEDAEELGIKVDEPRVYYGPLIASATDGA
DYAIVGDTGDGPVEYDTDTSSYTYEGAGGVDIGNMVNRAMFALRYQEMNMLLSDRVGSESKILFERDPR
SRVEKVAPWLTTDSKTYPTVIDGRIKWIVDGYTTLDSLPYSTRTSLTEATQDAVMPDGTPQPLITDRVG
YIRNSVKAVVDAYDGTVELYEFDTEDPVLKAWRGVFPDTVKDGSEISDELRAHLRYPEDLFKVQRDMLA
KYNVDDSGTFFTNDAFWSVPGDPTAAEGRQELKQPPYYVVAADPETGESSFQLITPFRGLQREYLSAHM
SASSDPVTYGEITVRVLPTDSVTQGPKQAQDAMMSSDQVAQDQTLWRGSNDLHNGNLLTLPVGGGEILY
VEPIYSQRKDQASAFPKLLRVLVFYKGQVGYAPTIAEALSQVGIDPKEAQDIEEVDGTATTPSTDETDT
DTDQPATETPTAPVSEAEGIAAINDALSNLEAARDSSFEEYGRALDALDRAVDSYQSAQ
>RXS01677 TRANSLATE OF: RXS01677.seq check: 5194 from: 1 to: 744
VNQQSKKWLVPTLVVIIAVLLIAVVLLMYRGNASDTAEGVSAAATSDSAAASTAASGSASGAADSDLTS
VEARDPSDPVAVGDVDAPVGLVVFSDYQCPFCAKWSDETLPQMMKHVEDGNLRIEWREVNIFGEPSERG
ARAAYAAGLQDAYLEYHNALFANGEKPSEDLLSEEGLIKLAGDLGLDESKFTADFQSPETAVAIAQHQQ
LGIDLGAYSTPAFLLGGQPIMGAQPASVFEAAFEQALAAKE
>RXS02586 TRANSLATE OF: RXS02586.seq check: 4914 from: 1 to: 270
MHLLRDDNWWAPGFVKKAYTVMGHGSEVEEAPRPTTRRLNDDEEVTVHEAVVAGDTVASRGGLSTQENR
DLVSFVELKARLEKRRLEDLD
>RXS02587 TRANSLATE OF: RXS02587.seq check: 637 from: 1 to: 2091
VFSKWGHFAYRFRRIVPLVVIAAILALFVIFGTKLGDRMSQEGWDDPGSSSTAAARIELETFGRDNDGD
VVLLFTAPEGTSFDDAEVFSSISGYLDGLIENNPDEVSHINSYFDTRNQNLLSKDGTQTFAALGLKGDG
EQTLKDFREIEDQLHPDNLAGGVTTEVAGATAVADALDEGMAGDISRAEVFALPFVAILLLIVFGSVVA
AAMPLIVGILSILGSLGILAILAGFFQVNVFAQSVVTLLGLGLAIDYGLFMVSRFREEMDKGTPVEQAV
ATTTATAGKTVVFSAAMVAVALSGLFVFPQAFLKSVAFGAISAVGLAALMSVTVLPSLFSMLGKNIDKW
SLRRTARTARRLEDTIWYRVPAWAMRHAKAVTVGVVLLLLALTVPLTGVKFGGINETYLPPANDTRVAQ
ERFDEAFPAFRTEPVKLVVTGADNNQLIDIYVQANEVEGLTDRFTAGATTDDGTTVLSTGIQDRSLNEQ
VVEQLRAISVPEGVEVQIGGTPAMEIESIEALFEKLLWMALYIVLATFILMALVFGSVILPAKAIIMTI
LGMGATLGILTLMFVDGVGASALNFSPGPLMSPVLVLIMAIIYGLSTDYEVFLVSRMVEARDKGESTDD
AIRYGTAHTGSIITAAALIMIVVCGAFGFSEIVMMKYIAFGMIAALILDATIIRMLLVPRRDAPASRRQ
LVGTRLR
>RXS02590 TRANSLATE OF: RXS02590.seq check: 3473 from: 1 to: 936
MGISLLSSLLKIHGFPVVADFFFALAVVVAIVIIGGWLIYRSPSFKTEVMPAWAMLSMGLIALGTASPV
VLGDDLWGFMFVCWSIGTAVGLVAYSLYITAILRSKAGTPTFAWGLPLVTPMVASTSAAQLHEHFELPA
MLWVSFGLFLLTLASAPAVFTRVYFYYFGPKAQGIPLMATPTSWIPLGMVGQSTAAAQLIGASFGSKTA
ITMGIIYGIIMGIFTIPLGAIAHFVFYRAVFKGATYSPTWWASTFPVGTLSLGAHFLSQSTGVEWFNYF
SLYLIALMLFHVIVSTIAGTIAVMRRIVGKLKSQLA
>RXS02932 TRANSLATE OF: RXS02932.seq check: 938 from: 1 to: 972
VSKTEEGRSAAIIIYAFPTFILLGAIIAFIFPEPFIPLTNYINIFLTIIMFTMGLTLTVPDFQMVLKRP
LPILIGVVAQFVIMPFLAIVVAKMFNLNPALAVGLLMLGSVPGGTSSNVIAFLARGDVALSVTMTSVST
IVSPIMTPFLMLMLAGTETAVDGGGMAWTLVQTVLLPVIIGLVLRVFLNKWIDKILPILPYLSILGIGG
VVFGAVAANAERLVSVGLIVFVAVIVHNVLGYVVGYLTGRVFKFPEAANRTMAIEIGTQSAGLASGMAG
RFFTPEAALPGAVAALVHNITGAVYVGLVRNRPLTKASRKKESVAVSS
>RXS03042 TRANSLATE OF: RXS03042.seq check: 1569 from: 1 to: 606
LVLAFLVLLLVFRSIWVPLIAALGFGLSVLATFGATVAIFQEGAFGIIDDPQPLLSFLPIMLIGLVFGL
ANDYQIFLVTRMREGFTKGKTAGNATSNGFKHGARVVTAAALIMVSVFAAFIAQDMAFIKTMGFALAVA
VFFDAFVVRMMIIPATMFLLDDKAWWLPKWLDKILPNVDVEGEGLSELHEARTEELKENVGVGA
>RXS03075 TRANSLATE OF: RXS03075.seq check: 8649 from: 1 to: 726
VAKFLYKLGSTAYQKKWPFLAVWLVILIGITTLAGLYAKPTSSSFSIPGLDSVTTMEKMQERFPGSDDA
TSAPTGSVVIQAPEGKTLTDPEVGAEVNQMLDEVRATGVLKDADSVVDPVLAAQGVAAQMTPALEAQGV
PAEKIAADIESISPLSADETTGIISMTFDADSAMDISAEDREKVTNILDEYDDGDLTVVYNGNVFGAAA
TSLDMTSELIGLLVAAVVLIVTFGSFIAAGMPLIS
>RXS03124 TRANSLATE OF: RXS03124.seq check: 3878 from: 1 to: 960
MTPTLASMIGLAVGIDYALFIVSRFRNELISQTGANDLEPKELAERLRTMPLAARAHAMGMAVGTAGSA
VVFAGTTVLIALVALSIINIPFLTVMAIAAAITVAIAVLVALSFLPALLGLLGTRIFAARVPGPKVPDP
EDEKPTMGLKWVRLVRKMPVAYLLVGVVLLGAIAIPATNMRLAMPTDGTSTLGTAPRTGYDMTADAFGP
GRNAPMIALIDATDVPEEERPLVFGQAVEQFLNTDGVKNAQITQTTENFDTAQILLPQNLMRSMSAPLR
LSQLFVQMLRPSLMTPARRMALLASPQFTMTSLLASATSWFLTF
>RXS03125 TRANSLATE OF: RXS03125.seq check: 4701 from: 1 to: 171
LVLAFLVLLLVFRSIWVPLIAALGFGLSVLATFGATVAIFQEGAFGIIDDPQPLLCF
>RXS03220 TRANSLATE OF: RXS03220.seq check: 3878 from: 1 to: 960
MGLREILSSKWLVRILLVGIGLGVAQQLTGINSIMYYGQVVLIEAGFSENAALIANVAPGVIAVVGAFI
ALWMMDGINRRTTLITGYSLTTISHVLIGIASVAFPVGDPLRPYVILTLVVVFVGSMQTFLNVATWVML
SELFPLAMRGFAIGISVFFLWIANAFLGLFFPTIMEAVGLTGTFFMFAGIGVVALIFIYTQVPETRGRT
LEEIDEDVTSGVIFNKDIRKGKVH
>RXS03221 TRANSLATE OF: RXS03221.seq check: 3878 from: 1 to: 960
MFRDPAPPSKGTTNLGDKMASTFIQADSPEKSKKLPPLTEGPYRKRLFYVALVATFGGLLFGYDTGVIN
GALNPMTRELGLTAFTEGVVTSSLLFGAAAGAMFFGRISDNWGRRKTIISLAVAFFVGTMICVFAPSFA
VMVVGRVLLGLAVGGASTVVPVYLAELAPFEIRGSLAGRNELMIVVGQLAAFVINAIIGNVFGHHDGVW
RYMLAIAAIPAIALFFG

### Appendix A: DNA Sequences

>RXA00001-upstream
TGTCATAGGCAGCACTCTAGATGGCGCACAGTGACTCACTTCACTGTTTCTCACAGTACG
GATCGTTCGGCACGTACCTGCCGATGGAGGAGATTCTGCA
>RXA00001
ATGGCAACCGTAACGTTCAAAGATGCTTCCCTAAGCTACCCGGGAGCAAAGGAACCCACC
GTCAAGAAATTCAACCTGGAAATCGCCGATGGCGAGTTCCTCGTCCTCGTCGGCCCTTCC
GGCTGTGGTAAATCCACCACGCTGCGCATGCTGGCCGGTTTGGAAAACGTTACTGACGGT
GCCATTTTCATCGGAGACAAGGACGTTACCCACGTTGCACCGCGTGACCGTGACATCGCC
ATGGTTTTCCAGAACTATGCTCTCTACCCCCACATGACCGTGGGCGAGAACATGGGCTTC
GCACTGAAGATCGCCGGCAAGTCCCAAGACGAGATCAATAAGCGCGTCGACGAAGCCGCC
GCCACTTTGGGCCTGACCGAATTCTTGGAGCGCAAGCCGAAGGCCCTGTCCGGTGGTCAG
CGTCAGCGTGTGGCCATGGGCCGCGCCATTGTTCGCAACCCGCAGGTCTTTCTCATGGAT
GAGCCGCTGTCTAACCTCGATGCCAAGCTGCGTGTTCAGACCCGTACCCAGATTGCAGCC
CTGCAGCGCAAGCTTGGGGTTACCACCGTTTACGTCACCCACGACCAGACGGAGGCCTTG
ACCATGGGTGACCGCATCGCGGTGCTGAAGGATGGCTACCTGCAGCAGGTTGGCGCGCCC
CGAGAGCTTTATGACCGCCCCGCCAACGTCTTCGTCGCCGGCTTCATCGGCTCCCCAGCC
ATGAACTTGGGCACCTTCTCGGTCAAGGATGGTGACGCTACCTCTGGTCACGCTCGCATC
AAGCTTTCCCCGGAAACCCTCGCGGCCATGACGCGGGAGGATAATGGCCGCATCACCATT
GGTTTCCGCCCGGAGGCACTGGAGATCATTCCGGAAGGCGAGTCCACCGATCTTTCCATC
CCAATCAAGCTCGACTTCGTGGAGGAACTCGGTTCCGATTCCTTCCTCTACGGCAAGCTG
GTAGGCGAGGGCGACCTTGGATCGTCCAGCGAGGATGTCGCCGAGTCCGGCCAAATCGTC
GTCCGCGCTGCTCCGAACGCCGCGCCTGCTCCGGGCAGTGTTTTCCACGCACGCATCGTG
GAGGGCGGCCAGCACAACTTCTCGGCGTCGACTGGCAAGCGCCTCCCT
>RXA00001-downstream
TAAGCCCGCGTACCGGCTACCCC
>RXA00002-upstream
CTGACTTCTTGGGCTTCGGTGCTGCAATATCTAGGTTCACGCCCCGGATGGCACCGGAGA
GGCTGCAGACAAGCTCGCTGCTGAGGATTCTCACCTGCAC
>RXA00002
GTGCTGCACCGCGAAGGCAAGGGTGGCCTTCTTGGCGCTTATATCGCCGGCTTCGAGTGG
GGCCTAGAGAAGGATTACCATGTTCTGTGCGAAATGGATGCCGACGGCTCCCACGCACCA
GAACAGCTCCACCTCTTGCTTGAGGAAATTGAAAAGGGCGCAGATCTGGTCATTGGCTCC
CGCTACGTACCGGGTGGAGAGACAGTGAACTGGCCTGCCAACCGCGAACTGCTGTCCCGC
TTGGGCAACAAGTACATTTCTGTTGCCCTGGGTGCCGGCATCAATGACATGACTGCGGGC
TACCGTGCTTTCCGGCGTGAGCTGCTTGAGCACCTCGACTTTGAGGAGCTTTCCAACGCC
GGATACATCTTCCAGGTGGACGTTGCCTTCCGCGCCATCAAGGATGGCTTCGATGTCCGC
GAGGTTCCGATCACCTTCACCGAGCGCGAGCTTGGTGAATCCAAGCTGGACGGCTCCTTT
GTCAAGGATTCCCTGCTCGAAGTAACCAAGTGGGGAGTGGCTCACCGCTCCGAGCAGATC
AGCGATTTCACATCGGAAGTATCCAAGATCGCCTCCCGCACGGTCAAGGACATGGAGCTT
GGGCCTAAGGCCACCACGGCCAAGAACGGTGTACCGGACTTCGTTTGCGAAGTCTCTAAC
CTAGCTAAAGGCACCTTCAAGAAG
>RXA00002-downstream
TAACTCGATGCCCGCGGCGTCTC
>RXA00089-upstream
ACCTCGTTTTGGCCGCAAGTTCTCGTTAGTTAAAAACTGTAGGAAACCAGGTTCCACACA
TGACACACAATCGGTTTAACAGGGAAGGGGATCGGCACTG
>RXA00089
ATGGCAACACCAGCATCGGCTCCCACTTCCGAACCACGTCTCAAACGCACCAGAGCCAAG
CTTTTTGATTGGAAGCTTCTCATCGGCATCATTTTCGTCGCCGGCCTCGTGGTGCTTTCC
CTCCTCACCGGCCAATACGACATTTTCGGTGGCGATGATGGCCAACTGATGTTCGAGGCA
GTTCGCATCCCGCGTACCGTTTCCCTCATTTTGTCCGGTGCAGCAATGGCGATGTGTGGC
TTAGTCATGCAGCTGTTGACCCAAAAGAAATTCGTGGAACCCAGCACCACAGGAACAACC
GAATGGGCAGGTCTTGGCCTGCTCTTCGTGATTTACTTCGTGCCAGCCGCGACCGTTTTG
GATCGCATGCTCGGTGCCGTGGTGTTTTCCTTCATCGGAACCATGGTGTTCTTCCTCTTT
CTACGCCGAGTAACACTGCGTTCCTCATTGATCGTCCCGATTATCGGCATCATGCTCGGT
GCCGTGGTGTCATCGATCTCCAGCTTCTTCGCCTTGCAATTCGACATGCTCCAGCAATTG
GGAACATGGTTTGCGGGTTCCTTTAATACAGTGTTCCGCGGACAGTACGAAGTGCTGTGG
ATCGTTGTCATCGTCGTTATTGGAGTGTTCTTCTTCGCAGACCGGCTCAGCGTAGCTGGC
CTTGGCGAGGAAATCGCGACAAACGTGGGTCTCAATTACAACCGCATGGTCCTTATCGGA
ACTGGCCTCATCGCCATCGCAACAGGTGTGGTCACCGTCGTGGTTGGTAGCCTGCCATTC
CTCGGACTCATCGTGCCCAACGTTGTGTCCATGTTCCGTGGCGATGACCTGCGCTCGAAC
CTGCCATGGGTATGCCTAACCGGCATCGCGATCGTAACCATTTGTGACTTGATCAGCCGA
ACCATCATCGCGCCTTTCGAAATTCCAGTTTCAGTAATCCTGGGCATCATCGGCGCAGTG
GTCTTCGTGATCATGATTGTGAGGCAACGTGGCCGTGGA
>RXA00089-downstream
TAAAGATATTGAAAACCGCACCT
>RXA00090-upstream
TTGATGAGCCGAACCATCATCGCGCCTTTCGAAATTCCAGTTTCAGTAATCCTGGGCATC
ATCGGCGCAGTGGTCTTCGTGATGATGATTGTGAGGCAAC
>RXA00090
GTGGCCGTGGATAAAGATATTGAAAACCGCACCTCAGACCTTTCTCGATGGGAAACTATG
GAGGAATCAGCAACGGTCGAGGGACGCACCGATGTCGAACTAGCATCAGCGCCGAGCAAA
CGACGCAGCTCAGGTGCATTCCAAAGAGCGCGCGGGAAGCGCCGCTACTGGATCATCATG
GCCGCGCTGCTGGTCACCGCGCTTGCCTTCACCTGGGGCCTCATTTGGTACAAGAACGCG
ATGCCCGTTGGGCATCCGGCCTTCGCGCTGATTGCAGAACGAGGCATGGAGTCGGTCTTT
GTCATGCTGATTGTTGCGGTTTGCCAAGGCTTTGCGACGGTTGCGTTCCAGACCGTCACC
AACAACCGCATTATCACGCCGTCGATCATGGGCTTTGAATCTCTCTACACACTGATTCAT
ACCTCCACAGTGTTCTTCTTCGGCGCAACTGCACTGCTGGCCACCAGAAATCTCGAAATG
TTTGTCGGCCAGCTGGTGATCATGGTTCTTTTGACCTTGGTCCTCTACACCTGGCTGCTT
TCCGGAAAACGCGGCGATATGCACGCCATGCTGCTTGTCGGCATCATCATTGGTGGCGGA
CTCGGATCCATCTCCACCTTTATGCAGCGCATTCTGACCCCATCAGAATTCGATATTCTT
TCCGCCCGACTTTTCGGATCAGTAAACAACGCGGAAACCGAATACTTCCCAATTGCTGTT
CCACTAGTAGTAGTGGCGTCCGTCTTGTTGCTGCTAAGCTCTCGACGCCTCAACGTTGTA
GGGCTTGGCAAAGATGCCGCAACCAACCTTGGAATTAATCACCGACGATCCTCCATTTAC
ACACTGGTTCTCGTCTCTGTATTAATGGCAGTATCCACCGCACTTGTCGGACCGATGACA
TTCCTCGGATTCTTGGTCGCGACCTTGGCATATCAATTCGCCGACACTTACGACCACCGA
TACATCCTTCCGATGTCCGCACTCATCGGATTCGTCGTACTCAGCGGCGCTTACTTTGTC
ATGAACCAGGTGTTGGGCGCAGAAGGCGTCGTGTCCATCATTATTGAGATGGTCGGCGGT
ACCGTGTTCCTCATCGTCATCCTCAGAAAGGGGAGACTG
>RXA00090-downstream
TGATTACGTTAAGTAATGTCGGC
>RXA00099-upstream
CTCTGGTGAAGAGGATGTTGAGTCGGGAGATTCTTCCACTGATTCACTGATTAAGTGGTA
CCGCGCAAATAGGTAGTCGCTTGCTTATAGGGTCAGGGGC
>RXA00099
GTGAAGAATCCTCGCGTCATAGCACTGGCCGCTATCATGCTGACCTCGTTCAATCTGCGA
ACAGCTATTACTGCTTTAGCTCCGCTGGTTTCTGAGATTCGGGATGATTTAGGGGTTAGT
GCTTCTCTTATTGGTGTGTTGGGCATGATCCCGACTGCTATGTTCGCGGATGCTGCGTTT
GCGCTTCCGTCGTTGAAGAGGAAGTTCACTACTTGCCAACTGTTGATGTTTGCCATGCTG
TTGACTGCTGCCGGTCAGATTATTCGTGTCGCTGGACGTGCTTCGCTGTTGATGGTCGGT
ACTGTGTTCGCGATGTTTGCGATCGGAGTTACCAATGTGTTGCTTCCGATTGCTGTTAGG
GAGTATTTTCCGCGTCACGTCGGTGGAATGTCGACAACTTATCTGGTGTCGTTCCAGATT
GTTCAGGCACTTGCTCCGACGCTTGCCGTGCCGATTTCTCAGTGGGCTACACATGTGGGG
TTGACCGGTTGGAGGGTGTCGCTCGGTTCGTGGGCGCTGCTGGGGTTGGTTGCGGCGATT
TCGTGGATTCCGCTGTTGAGTTTGCAGGGTGCCAGGGTTGTTGGGGGGCCGTCGAAGGTT
TCTCTTCCTGTGTGGAAGTCTTCGGTTGGTGTGGGGCTCGGGTTGATGTTTGGGTTTACT
TCGTTTGCGACGTATATCCTCATGGGTTTTATGCCGCAGATGGTAGGTGATCCTCAGCTC
GGTGCGGTGTTGTTAGGCTGGTGGTCAATTTTGGGATTGCCGCTGAACATTCTGGGACCG
TGGTTGGTGACGCGTTTCACTAACTGCTTCCCGATGGTTGTTATCGCCAGTGTCATGTTT
CTCATCGGTAATGGTGGGTTTTGTTTGGCTCCGGATGTTGCGCCGTGGTTGTGGGCGACG
TTGTCTGGTCTTGGTCCCCTTGCGTTCCCGATGGCGTTGACGCTCATTAATATTGGTGCT
GAAACTAGTGCTGGTGCTTCTGCGTTGAGTTCCTTCGGGCAGGGTTTGGGTTATACGATT
GCGTGTTTCGGTCCCTTGTTGACTGGTTTCATTGTCGATGCAACAGGCAGCTTCCGAACA
ATCTTTTTGCTTTTTGCGCGTGCAACACTCTTCGTTATTAGAGGCGGTTACTTTGCGACA
AGGCAGGTTTACGTCGAAAAGCTTTTAAATCGC
>RXA00099-downstream
TAGGATGGCGCTATGCCGCAAAG
>RXA00123-upstream
GAAGGATCGTCAGAATAGCTCTCGAATAGGCCATTTCTTACTTCATCGGCAATACTGACT
TAGTAGAAATTGCTGTCCAGAACTGTTGAAGGAGTTGAAA
>RXA00123
ATGCCAAAGAATTACGACATCAACGGGGCGATCCGCAGACGGGATATGCTCAGACGTCGG
TACCTTCCTGATTCGGCAAATTCAACTCCTCTACCTGAAGACGTTTCTCCGCTGACCCGC
TATGTCACCGACGGCATCCCGAAGCGCCCACCGCTGGGTGCCACTGTTGCTGACGGTTTA
AAATTCGCGGAAGGCGCCTCCAACCGCATGGTCATGTCGCTGTACCCTGCGGCATCCAAG
CCCGCAATCGAGGAATTGGCAGAGGCCTGGGACCTCCACCCCACCATCGTAGAAGACTTG
CTCCTTGGTCAGCAGCGCCCAAAACTAGACCGCTACGAAGACATCATTTTTATCGCGATC
CGCTCCGCGCGCTACATCGACTCCCGCGAAGAGGTGGACTTCTCCGAATTCCACATCCTC
ATGAAGCCTCAGGCCATAGCCATTTTGTGCCAGGATAACCAATGGATTGACGGCACCAGC
GCCGCCAGCTTCAGCAACCCCGAGGAGATCGATAAGCGCATAAAAACATTGCTTGCCGAC
GCCGAGTTACTCTCGTCCGGCCCCCGCGCCGCGGCCTATAGGCTTCTCGACGCCATCGTC
GACGGCTTCTCCCCCGTTCTTAGAGGCATCGCCATCGACCAGGAACAGATTGAGCGCCAG
GTGTTCTCCGGCGACGCCGCCGTCGCCGAACGTATTTACAACCTGTCCCAAGAAATCATC
GACATGCAGCACACCACCAGCTCAGTTACCGAAGTGGTGCAACGCCTCAACAAAGACTTC
ATCCGAAGTGGCATGTCCGAAGAACTCCGCGCCTACCTCGACGACGTCGCCGACCACCTC
ACCCGCGACAACACCCGCGTCTCCGAATACCGCGAATCCCTATCCCAAATTTTGAACGTC
AACGCCACCCTTGTAGCCCAACGCCAAAACGAAGACATGAAGAAAATCTCCGGATGGGCC
GCCATCATCTTCGCCCCAACCCTCGTGTCCTCCATCTACGGCATCAACTTCGACATCATG
CCAGAACTTCACTGGGCGTTTGGCTACCCGTTGGCTCTCTTAGCAATGCTCGGATTCACC
CTCCTTTTGTACTGGATCTTCAAACGCAGTAAGTGGATG
>RXA00123-downstream
TGAGACAAAAACCGAAAAACCAA
>RXA00160-upstream
TACAGGGGTGGGGTTACCCCCTAAGGTGGTCACAACTTGATAACGGACTGGTTAATAAAT
GGCCAATCTGACCATTTTAACCTCCATAAAAAGGATTCTC
>RXA00160
ATGCTAAACATCGCACGCAACCGCAACATGAAGCGTCGACTAGCAATTGCTGCTTTCGTC
GCCACCGCAACCGCTACCGCCACCATGGCACCAGCATCCGCGCAAACCGACTACGCAGGC
CTTTCCTCCGGCGTTGCCGACACCGTCGCAGAAGCTGCAGGAGTCGCAACCACCGCCGTC
GCACCAGCCGCCACCGTAGCGCGCCCAGCAAACGGCACCTTCACCTCAGGATTCGGACCA
CGTTGGGGAACCTTCCACAACGGCATCGACATCGCAAACTCAATCGGCACCCCAATCTAC
GCCGTCATGGCCGGCACTGTCATCAGCTCTGGCCCAGCATCCGGCTATGGACAGTGGATC
CGCATCCAGCACGACGAGGGATCCATCTCCATCTACGGACACATCGAATACCTCTACGTC
TCCGTCGGCGAACGCGTCGCAGCAGGGCAGGAAATCGCACGAATGGGCAGCCAAGGATTC
TCCACCGGCTCCCACCTCCACTTCGAGATCCACCCAGACGGCGTCACGCGAGTCGAGCCA
CAGGCATGGCTCGCAAACCACGGCATCTACGTT
>RXA00160-downstream
TAAGCGCTAGCCGTTCGTGGGAT
>RXA00193-upstream
CCTCAGCGTCCTCTCCTCAGCGCCTTCCCCGCTGGGAAACGTGTGGCAGCACCTGAAATT
AAGGTTTCACCACC
>RXA00193
ATGCAAGGAACGCTGAAGAAGTAGTTCCCAGTCTTTGTCTTGCCCACCCTTCTGGGATTC
ATGATTGCGTTCTTGGTGCCGTTCATCGTGGGTTTCTTCCTCTCCTTTACGAAGTTCACC
ACTATCACCAACGCCAAGTGGGTTGGCATAGACAACTACGTCAAAGCTTTCTCCCAACGC
GAAGGTTTCATCTCAGCCTTCGGTTTCACCGTCCTCGTGGTCATCGTCTCCGTGATCACA
GTCAACATCTTCGCCTTCCTCTTGGCGTGGTTGCTGACCCGCAAACTCCGCGGTACCAAC
TTTTTCCGCACAGTCTTCTTTATGCCGAACCTTATCGGCGGCATTGTGCTGGGTTATACC
TGGCAGACCATGATCAACGCCGTGCTTTCGCACTATGCCACGACTATTAGCGCGGACTGG
AAATTCGGCTACGCCGGCCTCATCATGCTACTTAACTGGCAGCTCATCGGCTACATGATG
ATCATTTACATCGCCGGCCTGCAAAACGTCCCACCAGAGCTCATTGAGGCTGCCGAACTC
GACGGCGTCAACAAGTGGGAGATGCTGCGGCACGTCACTATTCCGATGGTGATGCCATCC
ATCACCATCTGCCTCTTTTTGACTTTGTCGAACTCCTTTAAGCTCTTCGACCAGAACCTG
GCGCTGACCAACGGCGCTCCTGGCGGGCAAACTGAGATGGTGGCGCTCAACATCATCAAC
ACGCTGTTTAACCGTATGAATGTCGAGGGCGTCGGTCAGGCCAAGGCCGTTATCTTCGTC
GTCGTTGTGGTCGTCATCGCGTACTTCCAGCTGCGCGCGACCCGCTCCAAGGAAATCGAG
GCT
>RXA00193-downstream
TAAGTTATGACTACCAGCAGTTC
>RXA00203-upstream
AGGAAGCTGCTGCAGAAATCGAAAACACAAAGGAGGACCGTTGAGCACCGCCGTAGTTTC
ACAGAAGAAGTCGACAACGGCATCCAAAATTGGACATTGG
>RXA00203
ATGCTCAATAACGGTGCGTTGGTGGGGCTGATTGCACTGTGTGTTGGACTTTTTATTGCA
ACACCCCACTTTCTCACCATTCCTAACCTGATCAACATCGGTATCCAATCGGCGACGGTG
GCGATCCTGGCGTTCGGCATGACCTTCGTCATCGTTACCGCAGGCATTGATTTGTCTGTG
GGATCAGTGGCTGCGTTGGGTGCGATGACCTCGGCGTATTTCTTCGCGGAAGTTGGTTTG
CCGGGCTGGATCACGCTGCTGATTGGCCTGTTCATCGGATTGTTGGCGGGTGCGATCTCT
GGCATTTCTATTGCTTATGGCAAGTTGCCTGCGTTTATTGCCACCTTGGCCATGATGTCG
ATCGCCAGGGGAATCACCTTGGTCATTTCCCAAGGCTCACCAATTCCCAGTGCACCAGCT
GTGAACGCTTTGGGGCGCACCTAGTTTGGCATCCCGATGCCGATTCTGATGATGGCACTG
GCTGGCATTGTGTGTTGGTTTATTTTGAGCCGCACCGTGCTGGGACGGTCCATGTACGCC
ATTGGCGGAAACATGGAAGCAGCGCGACTATCTGGTCTGCGAGTGAAGAAAATCCTGGTC
ATGGTCTATGCACTGGCTGGTGTGTATGCAGCACTTGCGGGTCTGGTCATGACGGGACGC
TTGTCGTCCGCGCAGCCGCAGGCAGGCGTGGGATAGGAACTCGATGCGATTGCCGCCGTG
GTCATTGGTGGTGCGTCACTTGCTGGCGGAACCGGAAAAGCAACGGGCACTTTGATTGGT
GCCATCTTGTTGGCCGTGATGCGGAATGGCTTGAACATTTTGAACGTGTCCTCGTTCTGG
CAGCAGATTGTCATCGGTTGTGTCATCGGGCTTGCGGTGGGCTTGGATGTCATGCGAAAC
AAAACCTCTAAG
>RXA00203-downstream
TAATTCCTGAAAGGAAATTTTCA
>RXA00204-upstream
TCAACGGCGGTTTCCCAAAAGGCGGCCGCAAAGGCAGCAAAAGCAGCCCAAAAAGCAGCC
GCGAAAGCCGCACAGAACACGCAACACGAGGTGAGCCTAG
>RXA00204
ATGGTGAACTCTGAACAAGCGCTTCATCAGGATGATCCTGGACCAATCCTTCAGTTGGAT
AAAGTCTCCAAGTCGTTTGGCCCAGTCAACGTGATTAATCAAGTGAGCATCGATGTTCGC
CCTGGCAGGGTGCTTGCGCTGTTGGGTGAAAATGGTGCGGGTAAATCTACGCTGATCAAG
ATGATGTCGGGTGTGTATCAGCCTGATGGCGGGCAGATTTTGGTGGATGGAAAGCCCACG
ACTTTGCCTGATACGAAAACTGCTGAGTCTTTTGGCATCGCTACGATTCACCAGGAATTG
AATCTGGTGCCCACGATGACGGTGGCGGAAAACGTCATGCTGGGCCGCACTCCTCGGAAG
TGGGGTTTGGTCAATTTGAAACATTTGCGCAGGGAGGCACAGGCGGCGCTGGATCTCATC
GGCGTGGATGTGGATCTGAATGCTCAGGTGGGTTCTTTAGGAATCGCTAGGCAGCAGATG
GTGGAGATCGCGAAGGCGTTGTCCATGAATGGGCGGATATTGATTTTGGATGAGCCCACT
GCAGCGTTGACTGGTCGTGAAATTGATCAGTTATTCAAAGTGGTGGATGAGGTGAAAGAA
AAAGGCGTGGCCATGGTGTTTATTTCGCACCACTTGGATGAGATCGCGCGCATCGGCGAT
AGCGTCTCTGTGCTGCGTGATGGCGAGTTCATCGCGGAGCTGCCAGCGGATACTGATGAA
GATGAGCTGGTGCGGCTGATGGTGGGTCGTAGCATTGAAAACGAGTATCCGCGTAGTGCG
CGAGAGATCGGGCAGCCACTGTTGGAGGTGAAAAACCTCAACGCGGAGGGCCGGTTCACG
GATATTTCCTTGACTGTTCGCGCTGGTGAAGTCGTAGGCCTTGCCGGTCTTGTGGGTGCT
GGTCGCACGGAAGTGGTTCGCTCGATTGCTGGCGTGGACAAAGTTGATTCCGGTGAGGTG
ATCGTTGCTGGCAAGAAATTGCGCGGCGGCGATATTTCCGAGGCTATTAAAAACGGCATC
GGGCACATTCCGGAAGATCGAAAAGCCCAGGGCCTGGTGCTGGGGTCGTCTGTGGAGGAC
AACCTGGGATTGGCGACTTTGGCGTCGACAGCCCGCGCAGGTTTGGTCGATCGATCAGGA
CAGCACAAACGAGCCGCCGAGGTCGCGGAAAAACTCCGCATCCGGATGGCAAGCCTCAAA
CAACCGATTAGCGATTTATCGGGCGGCAATCAGCAAAAGGCCGTGTTCGGCCGCTGGGTG
CTTGCCGGGTCAAACGTGCTGCTTCTCGACGAACCGACCCGTGGCGTTGACGTCGGCGCG
AAGGTGGAAATTTACAACATCATTAATGAGATGACGGAAAAAGGTGGCGCTGTGCTCATG
GTGTCATCGGAGCTTCCCGAAGTCTTGGGCATGGCTGATCGCATTTTGGTCATGTCTGGT
GGACGCATCGCAGGCGAACTGCCAGCGAAGGGAACAACCCAGGACGATGTCATGGCTCTA
GCTGTTTCCCAGGTGGATGATTCCATCACCGAGGAAGCTGCTGCAGAAATCGAAAACACA
AAGGAGGACCGT
>RXA00204-downstream
TGAGCACGGCCGTAGTTTCACAG
>RXA00270-upstream
TGGAGACTGCAACTGAGTTCACCTACGTGATCAACGAAGATGCAGGAGAGCGCCAGGGGG
TGGAGATCCCTCAAGAGATTTTGGATAAGGCCGAACGCGT
>RXA00270
ATGATCGGCGCTTTTGAGTTCGGATTGTTGTACGGAGTTGTCGCATTGGGCGTCTATTTG
ACGTTCCGTGTGCTCAACTTTCCCGACCTCACCGTTGACGGCAGCCTGACCACTGGCGCG
GCAACAGCTGCGACAGCTCTTATGTCTGGCTGGCCTCCCCTTATGGCTACTGCCGCTGGT
TTCGTTACTGGCTTTATCGCTGGCATGATCACCGGTTTGCTGCACACCAAGGGCAAGATC
GATGGTTTGCTCGCAGGTATTTTGACCATGATTGCGTTGTGGTCGGTTAACTTGCGCATC
ATGGGTGGCGCGAACGTGCCATTGTTGCGCACCGATAACCTCTTCACCCCGCTTCGCGAC
GCCGGCCTCCTCGGCACATGGGCAGGCCCGGCGATCCTCGCCGTTGCAGTGGGAATTTTG
GGACTCATCGTCATCTGGTTCCTCAACACTGATATCGGACTGTCGCTGCGATCCACCGGC
GACAACGGGCCGATGGTGCAGTCCTTTGGTGTTTCAACGGATTTCACCAAAATCCTCACC
ATCTCCCTGTCCAATGGTTTTGTTGGTCTTGCCGGTGCACTCATCGCTCAGTACCAGGGC
TTCGCAGATATTTCGATGGGTATTGGCCTCATCGTGATCGGTCTCGCATCGGTTATTTTG
GGCCAGGCCATCTTCGGTCAGCGTCGCGTGTGGTTGGCTGTGTTGGCTGTCATCGTCGGT
GCCATCGCGTACCGCCTGATCATTTTCGCAGCACTGCGCGTTGGCCTTGACCCCAACGAT
ATGAAGGCAATTTCTGCGATCTTGGTGGTTGTCGCCATGCTGCTGCCGAGGTGGCGTGCG
AAGTTCTCCAAGGCACCGAAGCGTAAGCAACCAGTAGCAGTGGAGGCT
>RXA00270-downstream
TAAGACATGTTATCCATCAACGG
>RXA00311-upstream
CGGAGAGATCGGCATTTGGGCGACCGTGCTGTTGATGATCGCCCGCATCGCATAGGGATT
CTGTGCAGTGGCAGAAGCTGCAGGTGCATCCACACTGACC
>RXA00311
ATGGAACATTCTCGTGAAGGCAAGCGTGGATTCTTCACCTCATCGGTGATGGCGGGTTGC
TCAGTTGGAAACGTCGTGGCTGGCTTGGTATTTATCCCGTTCTTGATGCTGCCGGAAGAA
CACCTCATGTCATGGGGCTGGCGCGTACCTTTGCTGCTTTCCGCACTGGTTTTAGTTGTC
GCATACTTCGTGCGCACCCGACTGGAGGAAGCATGAACTGAGAAGGCGGAAGAGGACGCA
GGCGCTCCGGCTTTGGCTGTGCTGCGCACCCAGGGCATTGATGTCGCACGAGTTTTCCTG
ATCACCTTCTTCGCCGTTGTTCAGACCACTTTGAACGTTTACGCACTGGCATACGCCGCC
AACGAAATCGGCATCGATCGTTCCTTCATGGTGATGGTGAACACGATCGCGCTGGGGCTT
TCCATCGGAAGGATTCCTTTGGCCGCGTGGGTCTCTGACCGCATTGGCCGCAAGCCAGTC
TTGCTGTTCGGGGCCATCACCTGTGCAATCACCACCTACTTCTACTTCCAGGCAATCTCT
GAAGCTGACCTTGTGCTGATCTTCGCACTGTGCTTGGTCAACCAAGGTTTGTTCTACTCC
TGCTGGAACGGCGTGTGGACCATTTTCTTCCCAGAAATGTTCGCATCTTCCGTGCGCTAC
ACCGGCATGGCTATGGGCAACCAGCTCGGTCTGATCATCGTTGGTTTCGCACCAACCATC
GCCACCGCCCTGTACGCATGGAACGGTTGGGAAGCTGTTGCGGGATTCATCATCGGCGCA
ATCGCACTGTCTGCCGCAGTTATTTTGACCACCAAGGAAACCGCCTTCACCAAGCTTGAA
GATCTAGGGAAGAAA
>RXA00311-downstream
TAATGTCTGACAAGATCTGGAAA
>RXA00312-upstream
CCATTGAATGGGAAGAACTTGGTTGTGTGTTTGTGACACCTATTCTAAAGAACATTAAAC
GTGATTAAGTTCATGATTCTTAATGAGAAAGGGTGATCAC
>RXA00312
ATGGAAACCGTGAGGACCGCAACCGCCGCTCCTGAAACTGCATCTTTGAAGCTGCGTGAG
GCAGAAAGCCCAGCAAAGTCCCCAAAGAAAGCCGCCTTGGCGTCACTTTTGGGTTCGACT
CTGGAGTACTACGACTTTGTCATTTACGGCACCGCCTCCGCGCTGCTGTTCAATCACCTC
TTCTTCCCACAGGGCGACCCAGTCGTCGCGACGATCGGCTCTCTCGCCTCATTCGGTGTT
GCGTACATTGCGCGCCCCATCGGTGGTCTGGTGATGGGACATGTTGGCGATAAGATCAGT
CGCAAAACCGCCCTCATGGTGACGTTGATGATCATGGGTATCGCCTCCATTTCCATCGGA
CTTCTGCGCACCTACGGACAGATCGGCATTTGGGCGACCGTGCTGTTGATGATCGCCCGC
ATCGCA
>RXA00312-downstream
TAGGGATTCTCTGCAGTCGCAGA
>RXA00345-upstream
AGTGACCCATATGGCGTATCCGAGGTCTAACGCGATTTTGCGATTTTCAATAAGTTTTCA
TGTTGACATCCTTTTTCAATAAGCATTTA
>RXA00345
ATGGCAGGTATGAAAAAGCTTCTTTGGACACTCCCCATCCTCCCACTGGTACTAGCTGGC
TGCTCAACTGGATCAGCAGATTCCGCGGATTCCACCAACGCTGCCGGATCCAATTCCCTT
AAAGTGGTCACCTCCACCCAGGTGTGGGCTGACGTCGCCGAAGCTGTCGCCCCAGATGTA
GACATTGAAGCAATTATTACCGGTGGCGACATCGACCCTCATTCCTTCGAGCCTTCCGCT
ACCGATATGGCTAAAGTTTCCGAAGCTGACATCATTATCGTCGGTGGCGGCGGCTATGAT
TCCTGGCTCTACGGCACCTTGGAAGAGGATGATCGCATCATCCACGCATTGGATCTCTCA
GAGCATGACCACAGCGAGCATGATGATCACGAGCACGAAGCCGAAGAAGCCGACGAACAC
GACCACGATGAAGAGGGCCACGATCATGACGTCGACAACGAGCACGTCTGGTACTCCACT
GAATACGTCTCTGAGGTAGCTGAAGAGTTCGCAGAAAAAGTCACCGAGCTTGATCCCGAG
GCACAGGCCGATGCAACGGCTGTGACCACCAAGATGGACGAGCTGCACAATCAGATTCAC
GATCTTCCAGCAGTTCGCATTGCTCAGACCGAGCCGATCGCCGATCACATTTTGTCCCAC
TCCGACATGGTGGAATCCACCGGTGAGGGTTACCGCGCAACCACGTTGAGCGAGAGCGAG
GCAACGGCAGCAGATGTTGCGTCGTTCCAGGATGCAATTAACAACGGTGACCTGGATGTT
TTGATGTACAACCCACAGTCCGCGTCGACTGTCGCGACCAGCTTGAAGGATTTGGCAGAA
GAAAAAGGCATCCCAGTTGTTGAGATCTATGAGACCCCTCAAAACACCGAGAATTTCCTC
GATGCATTCACCAAGGCAGTTGATGATCTCACCGCTGCCACTAACCAGGTT
>RXA00345-downstream
TAGAATTATTTAAATGGTGTTGA
>RXA00378
AAATCCTGGCGGTCATATCCGTCCTGGTTCGCTTTTGACCACGGCACGTTGACCCAAAAC
GAGATTTATTTTGATGTGGCCTGCGGAATCACCGTGTTGCTTCTTGCCGGACGGCTGCTG
ACAAGGCGTCGAAGCCAATCCAGTTTGTTAGCGGAACTTGGTCGCCTCCAAATCGATCCA
CAGCGCATTGTCACTGTGGTGCGTAAACACCGATTGAAGCGCGTAGTCCAGGAACTGAAC
ATTCCAGTGCAGGAAGTCCGTGTCAATGACGATGTGAAAGTTGCACCTAATACCACGATC
CCTGTGGATGGCACTGTCATCGGTGGCGGTTCGCGGATCGCAGCTAGCATCATCATGGGA
CAAGACCAGCGTGATGTAAAAGTAAATGACAAAGTTTTCGCCGGCAGCCTCAACCTCGAA
TCCGAAATCAAGGTTGGTGTTATTCGCACTGGTCACCGCACCCGCATCGCCGCGGTACAT
AGGTGGGTTAAAGAAGCGACGTTGAAGGAAAACCGCCACAATAGGGCAGCGATCCGTTCG
GCCGGTAACCTTGTGCCCATCACGTTCACCCTTGCTGTGGTGGACTTCTGTCTGTGGGCA
CTGATCTCTGGAAACATCAACGCTGCATTTACCACTACCTTGGCTGTCCTTGCGTGGGTG
GCTCCGGTGGCCTTAGCGTTGTCTGCTCCACTTGGCACGAGGAATTCGATCGAAGCTGCA
GCACGACACGGTATTTTGGTCCGCTCTGGTGAAATTTTCCGAGTTCTCGATGATGTGGAT
ACTGCCGTATTTAATCGTGTGGGCACACTkACCGATGGCGAAATGACAGTGGAAACCGTC
ACAGCAGACAAAGGCGAGGACCCAGAACTAGTGCTGCGTGTCGCCGGGGCGTTGGCGATG
GAATCCCACCACGCGATTTCCAAAGCACTGGTGAAAGCATCCCGTGAAGCTCGTGATACC
GGCGCGGGTGGTGAAGATGTCCCACACTGGATTGAAGTAGGCAACGTGGAAATCACGGAA
GCCGGCTCATTCCAAGCAACCATCGAGCTGCCACTGATCAAACCATCTGGCGAAAAAATC
ATGCGCACCACAGAAGCACTCCTGTGGCGACCACGATCCATGACAGAAGTCCGTGAGCAC
TTAAGCCCCCGACTAGTGGCAGCAGCAACCTCAGGTGGCGCACCACTGATCGTGCGATGG
AAAGGCAAAGACCGCGGAGTTATCACTCTAAGTGACCACGTGAGATCAGATTCCTCCGAT
GCGATTATTGCGATTGAAGAACAAGGCATCGAGACCATGATGCTTTCACGTGATACTTAC
CCGGTGGCACGTCGATACGCAGACAGCTTAGGCATCACCCACGTCTTGGCCGGCATCGCG
CCGGGCAAGAAAGCCCAGGTCGTCCGTGCAGTCCACACCCGCGGATCCACTGTCGCGATG
ATCGGCGATGAATCAGTAATGGACTGTTTGAAAGTCGCTGACGTGGGTGTACTGATGGGC
GTCGATCGTCCCTCAGATCTGCGTGATGATTCCGATGACCCGGCAGCTGACGTTGTGGTC
ATGCGCGAAGAGGTCATGAGCGTGCCGACGCTGTTTAAACTGGCTCGACGCTACGCCAAG
TTGGTCAATGGCAATATTGCTCTGGCCTGGATCTATAACGGTGTTGCCATGGTGGTTGCA
GTGTCTGGCTTGCTGCATCCAATGGCTGCGACCGTGGCTATGCTGGCGTCTTCGCTGCTT
ATTGAATGGCGCTCGGGCAGGGCGCGCAAGTAC
>RXA00378-downstream
TAACCAGCAATTCCCAAGCCCAA
>RXA00412-upstream
CTTTTGACGAACACCACGTCGCGTACGCTTCCTCGGGGCGTTAAACTATTTGTCTTCCAG
CTTTTGTCCCCCGACTTTTGTACGAATCGAGGACACCGTC
>RXA00412
GTGTCACACACCGCGTCGACACCGACGCCAGAGGAATACTCCGCGCAGCAACCCAGCACC
CAGGGCACTCGCGTTGAGTTCCGCGGCATAACCAAAGTCTTTAGCAACAATAAATCTGCT
AAAACCACCGCGCTTGATAATGTCACTCTCACCGTAGAACCCGGTGAGGTAATCGGCATC
ATCGGTTACTCTGGCGCCGGCAAGTCCACTCTTGTCCGCCTCATCAATGGGCTTGACTCC
CCCACGAGCGGTTCGTTGCTGCTCAACGGCACCGAGATCGTCGGAATGCCGGAGTCTAAG
CTGCGTAAACTGCGCAGTAATATCGGCATGATTTTCCAGCAGTTCAACCTGTTCCAGTCG
CGTACTGCGGCTGGAAATGTGGAGTACCCGCTGGAAGTTGCCAAGATGGAGAAGGCAGCT
CGTAAAGCTCGCGTGCAAGAAATGCTCGAGTTCGTCGGCCTGGGCGACAAAGGCAAAAAC
TACCCCGAGCAGCTGTCGGGCGGCCAGAAGCAGCGCGTCGGCATTGCCCGTGCACTGGCC
ACCAATCCAACGCTTTTGCTTGGCGACGAAGCCACGTCCGCTTTGGACCCAGAAACCACG
CATGAAGTTCTGGAGCTGCTGCGCAAGGTAAAGCGCGAACTGGGCATCACCATCGTTGTG
ATCACCCACGAAATGGAAGTTGTGCGTTCCATCGCAGACAAGGTTGCTGTGATGGAATCC
GGCAAAGTTGTGGAATACGGCAGCGTCTACGAGGTGTTCTCCAATCCACAAACACAGGTT
GCTCAAAAGTTCGTGGCCACCGCGCTGCGTAACACCGGAGACCAAGTGGAATCGGAAGAT
CTGCTTAGCCATGAGGGACGTCTGTTCACCATTGATCTGACTGAAACGTGCGGCTTCTTT
GCAGCAAGCGCTGGTGCTGGCGAACAAGGTGCTTTTGTCAACATCGTTCACGGTGGCGTG
ACCACCTTGCAAGGCCAATCATTTGGCAAAATGACTGTTCGACTCAGCGGCAACACCGCT
GCGATTGAAGAGTTCTATCAAACCTTGACCAAGACCACGACCATGAAGGAGATCACCCGA
>RXA00412-downstream
TGAACGAGATGATCCTCGCAGCT
>RXA00413-upstream
CTGTACAGATCGCGGTCTATGGTGCTAATCTCCTAAATACTTGAATGACCATTTCATGAT
CCATTCACAAAAACTTTCCCAAACAAGGACGTATTTGAAA
>RXA00413
ATGAAACTTCGTCGCATCACAACCACCGCCATCGCTGGCCTCTTCGCCGCAACCGCACTT
GTTGCCTGTGGCTCCGATTCCGATGGAAGCAGCACCACTGTTGCTGAAGGCACCGAAGGC
GTGACCATGCGCATCGGCACCACCGACGCTGCGAAGGAAGCATGGACCGTATTGGAAGAC
AAGGCAGCTGAAGAGGGCATCACGCTGGACATCGTTCCTTTCTCTGACTACTCCACCCCA
AATGAGGCTCTTGCCCAGGATCAGCTGGACGTTAACCTCTTCCAGCACCTGAAGTTCCTG
GCTGAGTAGAACGTCGGCTCCGGCGCAGACCTCACCGCAGTTGGCTCCAGCGAAATCGTG
CCACTGGCACTATTCTGGAAGGACCACGACTCCATCGACGGCATTGACGGCGAGTCCGTT
GCCATCCCTAACGATCCTTCCAACCAGGGCCGCGCCATCAACGTTCTCGTTCAGGCAGGT
CTGGTCACCCTGAAGACCCCAGGTCTGGTCACCCCAGCTCCAGTCGATATCGACGAGGCA
GCTTCCAAGGTTTCCGTCATCCCAGTCGACGCAGCTCAGGCACCAACCGCTTACCAGGAG
GGTCGCCCAGCGATCATCAACAACTCCTTCCTTGACCGCGCAGGCATCGATCCAAACCTC
GCGGTCTTCGAAGATGATCCTGAGTCTGAAGAAGCAGAGCCATACATCAACGTCTTCGTC
ACCAAGGCTGAGGACAAGGACGATGCCAACATCGCCCGCCTCGTTGAGCTGTGGCACGAC
CCAGAGGTTCTGGCTGCAGTAGACCGCGACTCTGAGGGCACCTCCGTCCCAGTTGATCGT
CCAGGAGCTGACCTTCAGGAAATCCTTGATCGCCTTGAGGCTGATCAGGAAAACGCA
>RXA00413-downstream
TAATCTCTTTTGAGTTCTTTGCA
>RXA00431-upstream
TGGATCGTCCTCGCCTTCACATTCGTCGGCCTTGGCCTTGCTCTCCTCGCGATGAAGCAA
TGGCGATTCCGCGTCAGCTACTGGGTATAAGGAGCACCAC
>RXA00431
ATGGTATCGATCGATACATAGAACGCCTGCGTCGACTTGCCCATCTTCGACGCCAAATCC
GGCTCCATGAAGAAAGCCTTCCTCGGCGCAGCCGGCGGAGCAATCGGGCGCAATCAAGAC
AACGTCGTAGTCGTCGAAGCGCTGAAGAACGTCAACCTGCACTTGCGCGAAGGTGACGGG
GTCGGACTCGTCGGGGAGAACGGCGCCGGCAAATCCACCCTCCTGCGACTCCTCTCCGGC
ATCTACGAACCCACCCGCGGAAGCGCTGACATCCGTGGACGCGTCGCCCCCGTCTTCGAC
CTCGGCGTCGGCATGGATCCAGAAATCTCCGGCTACGAAAATATCATCATCGGCGGCCTG
TTCGTCGGTCAAACCCGCAAACAGATGAAAGGCAAAATGGAAGAAATCGCCGACTTCACG
GAACTCGGGGAATACCTCTGCATGCCTCTCCGAACCTACTCCACCGGCATGCGCATGCGC
GTAGCCCTCGGCGTGGTCACCTCCATCGAGCCCGAAATTCTGCTTCTTGATGAAGGCATC
GGCGCGGTCGACGCCGCGTTCATGGCCAAAGCCCGCGACCGGCTCCAAGCCCTCGTCGAA
CGATCCGGCATCCTCGTCTTCGCCTCCACTCAACGACTTTCTTGCCAACTCTGCAACACC
GCACTCTGGGTCGAC
>RXA00444-upstream
GATCGTGCCAAGGAGATCCTTGCCAGCnnnnnnnnTnnnAnTGGTTTTGGCACAAACTAA
AAAGGCTCGTCGAAGCGAGAATGATATCCTCCCAGGGTGG
>RXA00444
TTGCTGATCCCAGCCACCCTGGCCATGCTGCTGATCATTGGACCTATTTTTGCTTTGCTG
TTGCAGATCCCCTGGGATCGGTCTTGGGAGTTGGTTACCGCGCCGGAATCTTTAGGAACC
GCACGGTTATCTATCGGAACTGCTCTGTTTTCTACCGCGCTATGCGCAATTGTGGGTTTC
CCGCTAGCGTTGGCGCTGCATTTATATGAGGGTTCGCACCCCAGGGTGACATCAGTTTTG
ACGGTGCTGGTTTATGCGCCTTTGGTGTTGTCGCCGGTGGTGTCTGGTTTGGCGCTGACT
TTTCTGTGGGGCAGGCGTGGTTTTTTAGGTTCTTGGCTTGATCAGGTTGGATTGCCGATT
GCATTTACCACGACGGCTGTGGTGTTTGCCCAGGTGTTTGTAGCGTTGCCATTTTTCATT
TCCACTGTGACTACTGCACTGCGTGGGATTCCAAAACAGTTTGAGGAAATCGCAGCTACT
GAAGGCGGAACCGGCTGGGAGATCATGCACAAGATGATCATTCCGGTGGCGATGCCTGGA
ATTTTCACCGGTATGATTTTGGGATTCGCGAGGGCCTTGGGCGAGTATGGTGCGACACTG
ACTTTTGCTGGAAATATTGCAGGTGTTAGCCGCACCATTCCGTTGCATATTGAGCTTGGT
TTGAGTTCCAATGACATGGATAAAGCCTTGGGAGCGGTGATTATGCTTTTGGCTGTCTAT
GTCCTCATCATTGGAGCCATCGGAGCGTTACGATTGTTTTCCAAGGTGAGAAAGGTT
>RXA00444-downstream
TAATTGATGTGTCGTTCGCCGGA
>RXA00445-upstream
GGTGCAAAAAAGGACTAACC
>RXA00445
ATGGCGGATCTGAGCATTGAACACGTATCAAGGTTTTTCGGCGATGCCATCGCCTTGAAC
GATCTGTCATTGACCGTCCCCTCAGGCTCCATCACCGCCATCATCGGGCCGTCCGGGAGC
GGTAAAACCACGTTACTGCGTTTGGTGGCAGGCCTTGATTCACCCGATGAAGGCACCGTG
AGCATTGGGAATAAGATCGCCAAGCTGGGTGACACTGCGCTGTGTTTCCAGGATTCGCCT
TTGTATCCGCACCTTAATGTGTGGGAAAACGTGGCATTTCCGCTCAAGCTCAAAGCCACC
AATACTGCAGATGAGGTGGTGAAAAAGCGGGTGAGTGATGTTTTGGAAATGCTCGAAATT
GCTCCCCTCGCCCGCCGGAAAATTACCGAACTCTCCGGCGGGCAAAAACAGCGCGTCGGC
ATTGCTCGAGCACTGGTCAGAGACGTAGAGGTTTACCTTTTCGACGAACCGATGGCCCAC
CTCGACCAAGCCTTAGCCCGCGATATTGTGGCCGATCTGCGCAAAATTCAACAATCGTTG
GGACTGACGTTTGTATACGTCACCCACAGCAAATCCGAGGCATTCGCGCTCGCCGACCAA
ATTGTCGTGCTGGTAGATGGCCAAGTCGCGCAGGTTGGTGAGGCGGAGGACCTCGTCGAA
AAGCCAAAAACCCTAGAAATAGCCGAGTTCCTCTCCCCCACCGAGCTCAATGTGCGCCGG
CGTGGGGACGCCGTGGAGGCATGGCGACCCGAAGACACCCAGCTCGCCCGCGGTGGCACT
GCGACCGTGGAAGCCGTGACGTATTTGGGCCGCGAGTGGCTTGTACAAACCACCGAGGGG
CACGCCGTGTCGGAGGAAAAATTCGACGTCGGCGAAAGCGTCACGCTAACCCAGAAGAAG
GTGTTTAGTTTC
>RXA00445-downstream
TAGCCGCCTGCAAAAGGAGGGAG
>RXA00466-upstream
TTTAAAAGCGCACTAAGAGCTCGTCAATTCTTTAAAACAAGCTGAGAATGTGAATAATAG
GATAGGTTAACCTGATTCGATTAGAAAACGGAGATTTGTC
>RXA00466
GTGCAATCCCGCCTGTCCAAAATCCTGCGCAGTAGCGTCGTAGGCGTTGCTGTCCTAGCC
CTGTTAGCTGGGTGTTCTAACAATGCAGATGACACCGACGCTGATTCAACATCCACGGGA
AACTCCGCTTTTCCTGTTTCGATTGAACACGAGTTCGGAACCACCACAATCGATGATGTA
CCCGAAAGAGTTGTCACCCTTGGCGTTACCGACGCCGATATTGTCCTCGCATTGGGGACC
GTCCCAGTAGGCAACACCGGATACAAATTCTTCGAAAACGGATTGGGACCGTGGACTGAT
GAGTTAGTGGAAGGCAAAGAATTAACACTGCTTGACTCTGATTCCACACCAGATCTTGAA
CAAGTAGCAGCCCTGGAGCCAGACCTGATTATTGGAGTCTCTGCGGGGTTTGACGACGTT
GTATACGAGCAACTATCTGATATCGCACCGGTGGTCGCCCGTCCAGCGGGAACAGCTGCA
TACGCAGTAGCTCGCGAGGAAGCTACCAACCTTGTTGCCCGTGCGATGGGGCAATCAGAA
AAAGGACAAGAGCTCAATGAGGAAACAGATGCTCTGATCCAAGCTGCGCGTGATGAAAAT
CCTTCTTTTGACGGTAAAACAGGAACCGTCATCTTGCCATACCAGGGTAAATACGGTGCC
TACCTGCCAGGCGATGCACGGGGACAATTCCTCGATTCACTTGGCATTTCGCTGCCGGAA
GCAGTTCTTTCGCGAGACACCGGCGACAGCTTCTTTGTCGATGTCCCCGCTGAAAGCGTC
AAAGACGTAGACGGTGATGTTCTCCTCGTGCTTTCCAACGACGAAAATCTGGATATCACA
GCAGAGAATCCACTGTTTGAAACACTCAACGTTGTGCAAAAAGACGCAGTAATTGTGGCA
ACAACCGAAGAACGCGGGGGGATTACCTACAACTCAGTGCTGTCTGTTCCTTTTGCGTTG
GAACATCTCGCACCACGTATTGCTGAG
>RXA00482-upstream
GCGATTTCTATGAAATTCTTCACCCATCCACAACTATCTACTATACTTTTAGAAGTAATA
ACTATTGAGTTAATATAAACATGAAGAAAGGATTTGCTTT
>RXA00482
ATGCGCATTTCAAGCAAACTTGTCACCACAGCACTACTCGCAGCCATTTCACTTTTCGGG
ATATCCACGGCACAAGCCCAAGACATTTTTGACGGCGGACGACTTGCAGGTGGCTCGTCG
CAGGTATCTAACCTAAGTTCGGTTCCTGAAAACGTAGCGCTGCCCGAAATTGAAAATAGC
ATTGACCTAGAACGCTACAAAGGCAAGTGGTATCAAGTCGCAGCAATTCCCCAACCATTC
TCTTTACAGTGCTCACATGACGTTACCGCTGATTACGGCGTGATCGACTCGGACACAATC
TCTGTAACAAATAAGTGTGGCACTTTCTTTGGGCCTTCAGTTATTGAAGGCAGCGCTAAA
GTAGTTTCCAATGCTTCATTAAAGGTTAGCTTCCCAGGTATTCCATTTCAGAGTGAAGAC
AATCAAGCAAACTACCGCGTGACCTATATCGAAGATGATTATTCACTAGCAATCGTCGGC
AGCCCAAGCCGGTCCTCAGGATTTATACTATCCCGCACGCCACAGCTCAGTAGTGACCAA
TGGTCTCACGTTCGGAACATTACAGAGGACAGTGGGTGGTGGCCATGCGCATTCATTACA
GTCCCAGCGACAGGTGGCTTAAACACCGCCACTCCGCTCTGCACACTT
>RXA00482-downstream
TAATTAACGTAGATGGTCATCTA
>RXA00523-upstream
TGGCGCGGCGGGGGTGAATTTTTCAGACG
>RXA00523
GTGTTGCGTAATCAGTTGGCGTCGCCGGATATTATCGGCATTTCTTCTGGCGCGTCGGCG
GCGGGCGTAATTTGCATTGTGTTTTTCGGAATGTCGCAGTCTGCAGTGTCGGCGATTTCT
TTGTGTGCGTCCTTGGCTGTGGCGTTGTTGATTTATCTGGTGGCGTATCGCGGTGGTTTT
TCGGCCACGCGTCTGATTCTTACCGGCATTGGTATTGCTGCGATGCTGAATTCATTAGTG
TCGTATTCGCTGTCCAAGGCTGATTCTTGGGATCTGCCGACCGCGACGCGCTGGCTTACC
GGCTCGCTCAATGGTGCGACGTGGGATCGTGCGATGCCGCTGATTGTCACCACTGTGGTA
CTCATTCCGCTGCTGGTGGCTAATGCGCGCAATGTGGATCTTATGCGTTTGGGCAATGAT
TCCGCGGTGGGTTTGGGCGTTGCTACTAATCGCACGCGCGTCATTGCGATTATTGCCGCT
GTTGCGCTCATCGCCGTTGCTACCGCTGCATGCGGCCCGATCGCATTCGTGGCGTTTGTG
TCTGGCCCCATTGCCGCGCGCATTTTAGGCTCCGGCGGATCGCTCATCATCCCCTCCGCA
CTCATCGGCGGGTTGATCGTGCTCATCGCCGACCTAATTGGCCAATACTTCCTCGGCACC
CGCTACCCCGTCGGAGTTGTCACCGGCGCATTCGGCGCCCCATTCCTTATCTATTTACTC
ATTCGTTCCAACCGCGCGGGAGTAACCCTG
>RXA00523-downstream
TGACCACGAACCATCAACTATGC
>RXA00525-upstream
CCATCGTGTTTATTAGTCACAACCCTGAGCTTGCTGATGAATCTGATCGGGTGGTCACCA
TGGTTGACGGGCGCATCATTGGGTGTGAGGTGAkACACTC
>RXA00525
ATGAGCCTTGCAGAATCAATTCTTTTGGCGCTCACCAGCCTGAGAAGCAACAAGATGCGT
GCATTGTTGACGCTGTTAGGAGTCATCATTGGTATCGCATCAGTCATCGGAATTTTGACC
ATTCGTAAAGCCCTGCAGGATCAAACTTTGAATAGTTTGGAAAGCTTGGGCGCGAATGAT
CTGTCGGCGCAGGTGGAGGAACGCCCCGACGAAGATTCCCCCGAACCCGATATGTTCGCT
TTTTCTGGGGCTGCAAACTCTAGTGGCAATCTGATTCCGGAAGAAACAGTTGATACGCTG
GGCGATCGTTTCGCAGGCAGCATCACGGGAATCAGCGTTGGCGGAATGGGTACGCAAGGC
ACTCTCATCGGCGACAGCGCAGATCTTAAATCCGATCTCCTCGGCGTCAACGAGGATTAT
ATGTGGATGAATGGCGTCGAAATGAACTACGGCCGCGCCATCACGCAAGACGATGTTGCC
GCTCAGCGCCCCGTTGCGGTCATCGCCCCAGACACCTTTAATACGCTTTTCGACGCAAAC
CCCAACCTCGCTCTGGGGTCCGAAGTAGCTTTTGAACTCAACGGTCAAGAGACATTTTTG
CGGGTTATCGGTGTGTATAAAGAAGCCGCAGCAGGTGGACTTGTGGGAAGCAATCCAACC
>RXA00556
TACAGCCCATATACGGTGGGCAATGACATCACCCACACGAAAGATGGATTGAACACGTTA
AGTATCCGTGCAGCTCAGGGCGTAGACCAGGATTGACTTAAGGGTTCAGTGCAAACCTAC
TTCGACGCGCTGTACGCCAACAATGACTCGCACCACGTTGCCATGTTGGACTTCCGTAAA
CAGATCGAAGAGTTGAACACCATTCTCGGGGCAATGAGTTTGGGTATCTCAGCCATCGGC
GGAATTTCCTTGCTTGTCGGTGGCATCGGAGTGATGAACATTATGTTGGTGTCTGTCACC
GAGCGAAGCCGGGAAATCGGTGTCCGAAAAGCCCTCGGCGCTCGTCGACGTGACATTCGC
CTGCAATTCGTGGTTGAAGCCATGATGATTTGTTTCATCGGTGGCATCCTGGGCGTGCTT
TTGGGCGGCATTTTGGGATTGATCATGTCCAGCGCTATTGGCTACATTTCCTTGCCACCA
GTGAGTGGAATCGTGATCGCCTTGGTATTTTCCATGGCTATCGGCCTGTTTTTCGGCTAC
TACCCCGCCAACAAGGCAGCAAAGGTCGATCCAATTGACGCCTTGCGTTATGAG
>RXA00556-downstream
TAAAAGCCTCGTTTTTAAGGTAG
>RXA00596-upstream
GCGCCACCGACGGCCTCTTGAACACCGATGCATACCAACAGGCTGTGCTCGGTGAAAATG
CCATCGGAGTGCCAAGCCCTAGCTACCAGGGAGGAAACTA
>RXA00596
ATGCTTAACGCCCTGAAATTCATCCCATGGCTGATCGGCCAGATTTTCCTCTGTGGCTTC
AGCGTGATCACCGCTGCGGTAAAAAAGGACAGCGGCTTCAACCCCGTTGTTATCCGCTAC
GCACTTCGAGTGACCACGGACTTCCAGATCGCAGCCCTGTCAACGTGCATCACCGCGACT
GCTTCCACCCTGTCCCTTGGCCTACGCGAACCCCGCAAGCCCGGCGACCCCACCATTTTG
CTGATCCAAGCAGTGTTTGGTTCCGATCCAGTAGAAGTTTTTGAATCCATCGCCGATATG
GAACAACGCCTCGTCCCTTCGGTCGCTTCAATTGACCACGGCGTCCCAGGCCAAGGGGCT
TACAAGGAGATCGGCCCCAGCGATGCTGAGTGGCCAAGTCGCGAGATCGCTGACACCGCC
CAAAACACCGTCAGCCAAGACAAGAGGGAGTTT
>RXA00596-downstream
TAAAACAACATGACTGCTTTTGG
>RXA00634-upstream
AAGTGTTTTTAATTACCGCAGCTTTGTCTTAGGAGAAGTCATGGTCCAACTGGATCAACG
GGTTAGGTTCAAAAGCGCAACCTCAATAATCAGTATTGAA
>RXA00634
ATGTGGGAGCGATTCAGCTTCTACGGCATGCAAGCACTCTTGGTGTACTACGTGTATTTT
GATGTTGCAGCCGGTGGATTAGGCCTTGATCAAACCCAAGCAACAGGACTGGTCGGCGTT
TATGGCGCACTGCTCTACCTCTGCTGTTGGGCAGGCGGTTGGGTCAGTGACAGAGTCCTG
GGCGCAGAAAAAACCCTGCTGGGCGGTGCGATCTCAGTAACCATCGGACACCTTGTGCTT
GCTGGCCTCGGCGGGAAAATTGGTCTAGCCATTGGCCTTGGATGCATCGCGATCGGTTCA
GGATTTGTGAAAACAGCAGCCATGACCGTGCTGGGATGCAGGCATGGTGAACAAGAAGGA
GACGCAAAGGCAGATCCCGCATTCCAACTCTTCTACCTAGGCATCAACGTTGGTGGACTG
CTCGGACCACTCCTGACCGGTTGGCTCTCCAGCAGGTATTCCTTTGAAATGGGATTCGGC
GCAGCCGCAGTCGTTATGATCGGCGGATTGGGAATCTACGCAGCGTTGGGGAAACCAATG
CTGCAATCGTTCCCGCTCGAGGTGAAGAAAGCGCTGCTCCGCGCCCAAAACCCTGCAGAA
AAACATGTGATTAGCACGGCATTTGCTGCAGTGGCTGTGCTTTGCGGAGTGCTGCTTTAT
CTTCTCCTTACAGAAACAGTCAGCGCAGACCAACTAGCTGGAGCTCTGCTTTTAGTAACA
ATCGGTGCAGCACTATGGCTCATTATCCAGCCCTTACGACACCCACAAGTCAGCTCCGAA
GAGAAACGAAAAGTGCTGGCATTCATCCCGATCTTCGTCTGCTCAAGCGCATTCTGGGCA
GTGCAAGCACAAACCTACGGCGTACTAGCTGTGTACTCCCAAGAACGTGTTGACCGCATG
GTTGGCGATTTTGAGATCCCAGCAGCCTGGTCACAATCACTCAATCCTTTTTTCATCCTG
GCGCTGTCCATCCCGATTTCCCTGTGGTTTATGCGCGGATCACGCGCCCCAAGAGTGAAA
ATTGGAATCAGCATTGGAGTGATCATTGCGGGAAGTGGGCTTCTAGTTCTTATTCCATTT
GTTGGAATGCCGCTCGCGGCAGTGTGGGTGCTGCCTTTAAGTGTTTTCCTCATCTCACTG
GGAGAACTTTTCATCGGACCCGGAGGAATGGCTGCGACTGCGCACCACGCACCACGAATA
TTTGCCACACGATTCTCCGCCCTGTATTTCGTCACACTCGCCATCGGCATGTCTATTGCA
GGTAATGTGTCCAAATTTTACGACCCCAGCAACCACACCTCCGAGCTCCGATACTTCGCG
GTATTTGGCATTTCGATCATCGTCATCGGTGTCGGTTCACTGATGGTGGCCAAGAAGGTT
GGA
>RXA00634-downstream
TAACAGGGTTAATGTTGGGTGAT
>RXA00665-upstream
ACCAAACACTTCTGTGCGTGACACGCGCCACCTTATACTCCCACAAGCAACACAGAACAC
TCGGGATCTCAAAGTTTCGAGAAACACAGAAAGGGGAGCA
>RXA00665
ATGAGGAGCTCAACACTTCTCCTGGCTTCAGGACAAGTCACGGCATTAGCCGCTGACTAC
ACGCTCAGCCACACCCCCTCAGATGGCATCCTGGTAGTCCTTGGCTTCGCCATGATCGTC
ACCTTCATGACCCTGATCATGCTGGGTGGACTCACCCCAATGGTGGCCATGCTGTTGGTC
CCCACCATCTTCGGTCTCATCGCCGGCGCAGGACTCGGCCTTGGTGACATGGCGCTTGAC
GCCATCAAGGACATGGGGCCTACCGCGGCACTCCTGATGTTCGCGATTATGTTCTTGGGA
ATCATGATCGACGTGGGACTCTTCGACCCCCTGATCCGCGTGATCACCCGCGTTCTTCAC
GATGACCCCGCAAAGGTCGTCATCGGCACCGCAGTACTTGCAGGTGTTGTCTCCCTCGAC
GGCGACGGCTCCACCACC
>RXA00702
TTAGGGCTCCCACCTGCGGTGATGCGCAAGGGCGTAGAGGAAACCCTTGATCTTTTAGGC
ATGGCGGAGCTGCGATACGTGCCATTGGCGGAACTATCTGGTGGTGAGCAGCAGCGCGTG
GCGATTGGCGCGGTGCTGACCACTCGCCCCGCGCTGATTATCTTGGATGAACCAACCAGC
GCTTTGGACCCTAATGGTGCCGAGGATGTGCTGGCAACCGTAACCAAGCTGGCTCATGAC
TTGGCGATGACCGTAGTGCTTGCTGAACACCGCATCGAGCGCGTACTGCAGTACGTGGAC
CGCGTGGCGGATGTGGGCGCTGATGGGCACGTCACTGTTGGGACGCCGGAAGAAATCATG
GCTGATTCTGATGTGGCACCACCCATTGTGGAATTAGGACGCTGGGCTGGCTGGGCTCCC
CTACCGCTATCGATCCGCGATGCACGCGCACACTCCGCTGACATGCGCAAACGCCTGTAT
CAGCGTGGTTTAGTGGTGAACAAATTACACAACCACGCTGTCCAGCCACTTTTGATCGCC
GAAGATATCATGGTTGATTTCCCCGAAATCCGTGCCGTTGACGGCGTGAACTTGAATCTC
AACTCCGGTGAAATTACCGTGCTCATGGGCCGAAACGGCTGCGGAAAATCATCCCTGCTG
TGCGCTTTACAAGGTTCAGGGACTAGAAATCAGGGCTCGGTGCAGGTGCTTGATGAGGCC
GCGGGATTTTCGTGGACAGACCCCAAAACTTTAAAGCCCGCCAAGCGGCGCAATCTTGTG
TCCATGGTTCCGCAAACACCGACCGATATTTTGTATGAATCAACCGTGCATGCAGAGCTC
GCACGCTCTGATAAAGATGCCGCAGCACCCGCCGGCACCACGCGGGAAATCCTGGATTCA
CTGGTCCCGAATATCCCGGACCATCTCCACCCACGTGATCTATCAGAAGGCCAAAAGCTC
TCCCTCGCGCTGTCCATCCAACTCGCCGCAAAACCCCGCGTGGTATTTTTCGACGAACCC
ACCCGCGGCCTAGACTACGACGGCAAGAAATCCCTCGCCCGCTCCTTCCAACAACTCGCA
GACGACGGCCACGCCATTTTGGTGGTCACCCACGACGTGGAATTCTCTGCACTGTGCGCC
GACCGAGTGTTGTTTATGGCCTCTGGAAAGATCATCTCCGATGGCACAGCCGTAGAAATC
CTCCCCGCATCACCGGCTTACGCCCCACAAGTCGCAAAAATCACCGCCGGCATCCAAGAG
GAATCACACTGGCTCACAGTCTCGGCCGTGAAAGCTGCGCTAGGGCATGGTGAAATCTCA
>RXA00702-downstream
TGATCAACGCCATCACACTCAAG
>RXA00728-upstream
GATTACTTCACAGATGTGAGATCTTAATCAAGGGCCTGGAGCTTCAACGGCCCAACGGAA
ACCGATTGAAGCCAAGCCACTACGCCACCCTGGCCGGTGG
>RXA00728
GTGGCAGCCGCTATCATCGTGGCACTGCTCGCATGGTTTATCATCAGCGCGCTCAACAAT
GAGGCCTACGGTTGGGATACCTACCGCTCGTATCTTTTTGACACCCGCATTGCCACCGCG
GCACTTCACACCATTGCGCTGACCTTGCTGTCCATGATCTTGGGTGTGGTTCTCGGCGCA
ATCTTGGCCGTCATGCGTATGTCCGGCAACCCTGTCATGCAGGGCGTAGCGTGGCTGTAC
CTGTGGATTTTCCGCGGCACCCCAATTTATGTGCAGTTGGTGTTCTGGGGCCTGCTGGGT
TCCTTGTACCAGTCGATCAACCTCGGTTTCGCAGAGATCGATCTGCAAAGCTTGCTGTCT
AATATGTTCCTGCTCGCGGTGATCGGTCTGGGTCTCAACGAGGCTGCGTACATGGCGGAA
ATCGTGCGCTCGGGCATCCAAGCGGTGCCTGAGGGCCAGATGGAGGCGTCGAAAGCTTTG
GGTATGAACTGGTCAATGACCATGCGTCGCACCATCTTGCCGCAGGCCATGCGCATCATC
ATTCCGCCAACCGGCAATGAACTGATCTCCATGCTCAAGACCACCTCTCTGGTTGTTGCG
ATTCCTTATTCTCTCGAGCTGTACGGCCGCAGCATGGATATTGCGTACTCCCTCTTCGAG
CCAGTTCCAATGCTTCTGGTTGCTGCGAGCTGGTACTTGGTCATCACGTCTATTCTTATG
GTTGGTCAGTACTACCTGGAGAAGCACTTCGAAAAGGGCAGCACCCGCACCCTGACCGCA
CGTCAGCTCGCT
>RXA00732-upstream
TCTTTGGCAAGGTAGTGAACTTCTCTGAGCGTGAGATGGGTCAATTTGGCGCACCCGTCG
CTGATCACCGGGAAACACCAACGATGTGCAGCAGGTTCAG
>RXA00732
ATGCTGGTGCAGATGACCTCCACTTTGATGATTTGCGCCCCGATGCTGGCCATTGGTGGC
ATCATCATGGCGGTGCGTCAGGATCTTGGTTTGTGTTGGCTGATGGTGGTCAGTATTCCG
GTGCTGATCATCGTGGTGGCGCTGATCATTGTGCGCATGGTTCCGTTGTTCCAAACCATG
CAAAAGCGCATTGACCGCATCAATCAGATTATACGCGAGCAGCTCACCGGTATCCGCGTG
ATCCGCGCGTTCGTGCGTGAAGATGTGGAACGCGAACGATTCACCACTGCTAGTAAAGAT
GTCGCTGATATCGGCGTGCGCACCGGTAACCTGATGGCGTTGATGTTCCCTGCCGTGATG
CTGATCATGAACCTTTCTGCCGTTGCTGTGATTTGGTTTGGTGCTTTCCAGGTGGAATCC
GGCGAGACGCAGATCGGTACGCTCTTTGCATTCTTGCAGTACATCATGCAGATCCTCATG
GGCGTCATGATGGCAGCGTTCATGTTTGTCATGGTTCCGCGCGCTGCCGTTTCCGCTGAT
GGCATCGGTGAGGTTCTGGAAACCACACCGTCTGTGCAGGCGCCAGAAACACCGGCGCAG
CCGTCGACAAGCGCTGGCGAAATCGTGTTCAACAACGCGACTTTTGGCTACCCCGGCGCG
GATGACGCCGTGTTAAATAATGTGAGCTTCCGCGTTGCGCCTGGTAGCACGACGGCGATC
ATCGGCTCGACGGGTTCGGGTAAGACGAGGTTGATCGGGCTGGTTCCTAGGCTTTTCGAC
GTCACCGAAGGCGACGTTACCGTCGATGGCACCGATGTTCGT
>RXA00734
AGGCAGCTGCGTTATGGCAATGAAGATGCCACGGAAACGCAGCTGTGGCAGGCGCTTGCA
ATTGCTCAGGCGGCGGACTTTGTGCGTGAGATGCCAGAGGGTCTTGATTCTGAGATTGCT
CAGGGTGGAACCAATGTTTCTGGTGGTCAGCGCCAGCGACTAGCCATTGCCAGGGCGTTG
TTGAAGCAACCTGAGATCTATATTTTCGACGATTCTTTCTCCGCCCTCGATGTGAGCACA
GACGCGGCTCTTCGCCGAGCGCTGAGCACCAACCTGCCGGATGCAACCAAGTTGATTGTC
GCCCAGCGTGTCAGCACGATTCGAGATGCCGATCAGATTGTGGTGCTTGATAACGGCGAG
GTTGTCGGTATTGGAACGCACACGAATTTGCTGAACACGTGCGGTACCTAGCGTGAAATT
GTTGAATCCCAAGAGACTGCGCAGGCGCAATCA
>RXA00734-downstream
TGAGTAATACTGCAGGCCCCCGC
>RXA00759-upstream
TCACCTTGAACACTTAAAACATAACTTCATCCGGCGCTTTATTAGCTTGAAGCGCCCCGC
ACCATAATCCATTCCCCAGCAAGCAAGGACACCCACGCTC
>RXA00759
ATGCTTCGTTACGTCGGGCGACGTTTGCTCCAAATGATTCGGGTCTTTTTCGGAGCGACC
TTACTGATTTACGCCCTCGTGTTCCTCATGCCTGGTGACCCAGTCCAGGCATTGGGAGGT
GACCGCGGCCTAACCGAGGCTGCGGCCGAGAAAATCCGTCAAGAATACAATGTTGATAAA
GCGTTCATCGTTCAATACCTCGTGTACATCAAGGGCATCTTCGTGTTAGATTTTGGAACA
ACCTTCTCTGGTCAGCCAGTTATTGATGTGATGGCCAGGGCCTTCCCCGTCACCATCAAA
CTCGCCATCATGGCCCTGCTGTTTGAATGAATCCTCGGCATTATCTTTGGTGTCATCGCA
GGTATTCGCCGCGGAGGAATCTTCGACTCCACCGTGCTGGTCCTTTCTCTGATAGTCATC
GCAGTCCCCACCTTCGTCATTGGTTTCGTGCTGCAGTTCTTANTCGGCGTGAAATGGGGC
TTACTGCCCGTCACCGTAGGTTCCAACACATCAATAACGGCGCTGATCATGCCGGCTGTC
GTACTGGGTGCAGTATCGTTCGCCTACGTTCTTCGCCTCACCAGACAATCCGTGAGCGAA
AACCTCCGCGCTGATTACGTTCGAACCGCTCGAGCAAAAGGCATGTCCGGATTCAACGTG
ATGAACCGCCATGTGCTTCGAAACTCACTGATTCCCGTTGCCACCTTCCTGGGCGCCGAT
CTCGGTGCACTGATGGGTGGAGCGATTGTCACCGAAGGTATCTTCGGCATCAACGGTGTC
GGTGGAACGCTCTACCAGGCCATTTTGAAAGGTGAACCCACCACGGTTGTCTCCATTGTC
ACTGTGCTGGTCATCGTCTACATCATCGCCAACCTTCTCGTGGACTTGATCTACGCCGTT
CTCGATCCGAGGATCCGCTATGCC
>RXA00759-downstream
TAATAATGAATTCCACACAAACC
>RXA00760-upstream
ACCACGGTTGTCTCCATTGTCACTGTGCTGGTCATCGTCTACATCATCGGCAACCTTCTC
GTGGACTTGATCTACGCCGTTCTCGATCCGAGGATCCGCT
>RXA00760
ATGCCTAATAATGAATTCCACACAAACCACTCGTTGGGCCAAGATGATCAAACCCCAGAT
CAGGCTCATTTCTTCCCACAAGGACGAGGCGAGGCTCTAGTTCGACCAGGTGAAGAGCAC
TTCATCGCAGCGACTGATGAAACGGGACTTGGTGCCGTCGATGGTGTTGCTGATGACTCT
GCACCAAGCTCCATGTGGGGGGAAGCGTGGCGAGACCTTCGTCGTCGACCACTGTTCTGG
GTCTCTGCGGTGTTGATTATTTTGGCGCTTCTCCTGGCCGCAGTTCGGCAGCTGTTTACC
TCAACGGATGCGCAGTTCTGTGTGCTGGCAAACTCTCTTGATGGTCCACAGTCTGGACAT
CCCTTCGGATTCGACCGTCAAGGTTGCGATATTTTTGCTCGTACCGTCTACGGTGCTCGT
GCCTCGGTCGCCGTCGGTGTGTTGACCACGTTAGTGGTCGCCCTCATCGGTAGTGTATTT
GGTGCTTTGGCTGGCTTCTTTGGTGGCATCATGGATACCATCCTCTCCCGCATCACCGAC
ATGTTCTTCGCCATTCCACTGGTTCTGGCAGCCATCGTTGTGATGCAGATGTTCAAGGAA
CACCGCACGATCGTCACCGTGGTTTTGGTGCTTGGGCTTTTCGGCTGGACCAACATTGCG
CGTATTACCCGTGGAGCGGTGATGACCGCAAAGAATGAAGAGTATGTCACCTCCGCACGT
GCGCTTGGTGCATCAAAAGCCAAGATACTGCTGTCTCACATCATGCCAAACGCCGCAGCA
CCCATCATTGTGTATGCAACTGTGGCACTGGGAACATTCATCGTGGCAGAGGCGACGCTC
TCCTTCCTGGGCATTGGCCTTCCACCATCAATTGTCTCCTGGGGTGCTGATATCGCGAAG
GCACAAACGTCCCTTCGTACCCAACCCATGGTGCTGTTCTACCCCGCAATGGCACTTGCA
CTAACCGTTTTGAGCTTCATCATGATGGGCGATGTCGTCCGCGAGGCTCTGGATCCTAAG
TCGAGGAAGCGA
>RXA00760-downstream
TGACCACCAACATCCCACAAACC
>RXA00761-upstream
TGCTGTTCTACCCCGCAATGGCACTTGCACTAACCGTTTTGAGCTTCATCATGATGGGCG
ATGTCGTCCGCGACGCTCTGGATCCTAAGTCGAGGAAGCG
>RXA00761
ATGACCACCAACATCCCACAAACCCCCAACCACGAGGGTGAACAGCCACTGCTCGAGCTG
AAGGATCTAAAGATTTCCTTCACCTCCTCCACCGGTGTTGTCGACGCTGTCCGTGGCGCA
AACCTCACCATTTATCCTGGCCAATCTGTTGCCATCGTGGGTGAATCCGGTTCAGGTAAA
TCGACCACGGCAATGTCGATCATCGGTCTGCTTCCAGGCACCGGCAAAGTGACCGAAGGT
TCCATCATGTTTGATGGCCAAGACATCACAGGCTTGAGTAACAAGCAGATGGAAAAGTAC
CGCGGTTCAGAAATCGGACTGGTCCCCCAGGATCCGATGACCAACTTGAACCCGGTGTGG
CGCATCGGCACCCAGGTCAAGGAATCCCTCCGAGCCAACCACGTGGTTCCAGGCTCAGAG
ATGGACAAGCGCGTGGCAGAAGTTCTGGCCGAGGCAGGTCTTCCTGATGCTGAGCGTCGC
GCAAAGCAGTACCCACATGAGTTCTCTGGCGGTATGCGCCACCGCGCACTGATCGCCATT
GGTTTGGCGGCACGCCCGAAGCTCTTGATCGCCGACGAGCCCACGTCTGCG
>RXA00774-upstream
ATTATCGGCTTATAGTGTTGCCATGGGTACTGCATATAGACAGCAATTGGATGAATTCGC
ACACAATCTAATCATTTTGTGTGATCTAACTAAGGAGTGC
>RXA00774
ATGGATAAGGCGACTGATGCCCTCCTGCGCACTTCTTTGGCATCGGCAGAAAGCGCTTTA
GGCAATGCAGAAAAGCTTGAAGAGCTTCGTACTGGATGCGAGTCTCAAGCCGTCGAACTT
TTGGCGCTTGAAACTCCTGTAGCCCGTGATCTTCGCCAGGTTGTCTCCTCCATCTACATC
GTCGAGGAAATTACCCGTATGGGTGCTCTGGCAATGCACGTGGCTAATTCCGTGCGCCGC
CGTTACCCCGATCCGGTGATCCCGGAGGACATGCGTGGCTATTTCAAGGAGATGGCCCGC
CTCGCAGCTGACATGACAGATCATATTCGTCAGATCCTCATTGATCCTGAACCAGATCTT
GCCCTAGAGATGGCTAAAAGCGATGACGCGGTGGATGATCTGCATCAGCACATCATGCGT
ATTCTCACGCTGCGTCCTTGGCCTCACGACACCAAGAGCGCGGTTGATTTGACGCTGCTT
TCCCGCTTCTACGAGCGTTACGCCGATCACACGGTAAACGTGGCCGCCCGTATCATTTAC
CTGTCCACCGGGCTGCACCCGGAGGAGTACATGGAAAAGCGCGAGCAACAAAGGGCCGAT
GCCGACATGGAGAAGCGCTGGGCCGAGCTGGAGCGGCAGTTCCGCACCAGCGAG
>RXA00774-downstream
TAAAAAGGTGCTTCTCGACGCTA
>RXA00775-upstream
TCATGATCGCTGTCGTGAACATTGGCGCACGAATCATCTCCGCCAAGTTCTCTGTCAAGC
AATAATAATCTCAGAAAATACAACAGGAGTATCTAAAGCG
>RXA00775
ATGTCGAAGCTCAAGCTCAATGATGTCAACATCTACTAGGGTGATTTCCACGCAGTGCAG
AACGTGAACCTCGAGGTTCGTGCAGGCTCTGTCACCGCATTCATGGGACCATCCGGCTGT
GGCAAGTCCACAGTTCTCCGCTCCATGAATCGTATGCACGAGGTCACCCCAGGTGCATAC
GTCAAGGGCGAGATCCTTCTCGACGGCGAGAACATCTACGGCTCCAAGATCGACCCAGTT
GCAGTCCGTAACACCATCGGCATGGTGTTCCAGAAGGCTAACCCATTCCCAACCATGTCC
ATCGAGGACAACGTGGTTGCAGGTCTGAAGCTTTCCGGGGAGAAGAACAAGAAGAAGCTC
AAGGAAGTTGCTGAGAAGTCTCTTCGTGGCGCAAACCTGTGGGAAGAGGTTAAGGATCGT
CTGGACAAGCCAGGCGGCGGCCTCTGCGGTGGTCAGCAGGAGCGTCTGTGCATCGCTCGC
GCGATCGCGGTTGAGCCAGAAATCCTCCTCATGGACGAGCCTTGCTCCGCGCTTGACCCA
ATCTCAACCCTGGCTGTGGAGGACCTTATCCACGAGCTGAAGGAAGAGTTCACCATCGTC
ATCGTGACCCACAACATGCAGCAGGCTGCACGTGTGTCCGATCAGACCGCGTTCTACTGC
CTGGAGGCGACCGGTAGGCCAGGTCGTCTGGTTGAAATCGGACCTACCAAGAAGATCTTC
GAAAACCCAGATCAGAAGGAAACCGAAGATTACATCTCCGGCCGCTTCGGA
>RXA00775-downstream
TAAATCGAAGAATTAAAGCCACT
>RXA00776-upstream
CGTACATCTCCGCCGGCCTCGTGCTGTTCGCCCTTACCTTCATCGTCAACGCTGGCGCTC
GCGCCATGGTTAACCGCGGAAAGTAGAAGGGGACAAAATC
>RXA00776
ATGACTAACAATGTTGTTACTCCGCGCATGGATGAGCCTTTAAAGAAGAGCTCAGCCTTC
ACCGACATCTCCTCCAGCCGTAAGACCACCAACACCGCAGCAACCGTCATCATTTATGGT
GCGATGCTCATCGCAGCTGTGCCACTGGTTTGGGTGCTGTGGACCGTGATCTCTCGAGGC
ATCGCTCCGATCCTCACTGCTGATTGGTGGTCCACCTCCCAGGCTGGCGTCATGCTGATG
CTGCCAGGCGGCGGTGCAGCTCACGCCATGATCGGTACCTTCATGCAGGCGGTAGTCACC
TCGGTGATTTCCATTCCAATCGGTATCTTCACCGCAATCTACTTGGTGGAATACTCCAAC
GGTAACCGTCTCGGACGCTTGACCACCTTCATGGTTGACATCCTCACCGGTGTTCCTTCC
ATCGTTGCGGCACTGTTCGTGTACTCCTTGTGGATCGTGCTCTTCGGCTTCGACCGCTCC
GGCTTCGCAGTGTCCCTGTCACTGGTGATTTTGATGGTTCCAGTGATCATCCGAAACACC
GAAGAAATCCTCCGCGTTGTTCCTCAGGATCTGCGTGAAGCGTCCTACGCACTGGGCGTG
CCAAAGTGGAAGACCATCGCAAAGATCGTTCTCCCAACCGCACTGTCCGGTATCGTCACC
GGCGTCATGCTCGCAGTCGCTCGTGTCATGGGTGAGTCGGCACCAGTTCTGGTCTTGGTT
GGTTCCTCCCAGGCCATCAACTGGAACCCATTCGGCGGTCCGCAGGCTTCCCTTCGACTG
ATGATGCTTGATATGTACAAGGCCGGCACCGCACCAGCAACGCTGGACAAGCTGTGGGGC
GCAGCCCTCACCGTGGTGCTCATCATCGCTGTCCTGAACATTGGCGCACGAATCATCTCC
GCCAAGTTCTCTGTCAAGCAA
>RXA00776-downstream
TAATAATCTCAGAAAATACAACA
>RXA00777-upstream
TTAAGTGAATCGGGCGCCCTCTCCCAGCAATTGAGGGTAGGGCGCCCGATTTTACTAACA
AGCTTTTTATCAATACGCCAGTTAAGGAAATAAACCACCA
>RXA00777
ATGGCCACTAATGAGTCAGTCTCGGAGAAGCAACGCCTGGATGCAACCAGGGTGCAGGCA
CATCCTGTAGCAGTTAATGCGAACTCCTCTCAGACCAAGCCTTCAAAGAAGATTGTCGCC
GAAGGTGGCGGAAGCGTTAAGCGTCCCGGCGATCGCATCTTCGAAGTCCTATCCACCGCT
TCTGCAGCAATCATTACTGCGATAATGATTGCCATTGCGGCGTTCCTTATCTGGCGTGCT
GTTCCCGCCTTGATGCGAAATGCTGAAGGTATTGGCGGATTCTTCACTTATTCAGGCGCT
TGGAACACCACCGACATTGATGCAATGTACTTCGGTATTCCAAACCTGCTAGCTGCAACA
CTTCTCATCTCTGTCATGGCACTGATCATCGCCATGCCGATTGCTCTTGGTATTGCGATC
TTCTTGTCCAACTACTCACCAAAGCGCCTGGTTAAGCCACTTGGCTACATGGTGGACATC
CTGGCTGCTGTGCCTTCCATCGTCTACGGCCTTTGGGGCTGGCAGGTGCTCGGACCAGCT
CTGTCCGGTTTCTACACCTGGATTGAAAGCTGGGGCGGAAGCTTCTTCCTCTTCGCTACT
TACCAAAACTCACCTTCTTTTGCTACCGGCCGTAACATGCTCACCGGTGGCATGGTGCTC
GCAGTGATGATCCTTCCTGTTATCGAAGCAACCGCACGTGAAGTTTTCATAGAGACTCCA
AAGGGCCACATTGAATCTGCTCTTGCACTTGGCGCAACCGGCTGGGAAGTCGTTCGTTTG
ACGGTTCTCCCATTCGGAATGTCCGGCTACGTTTCCGGCGCGATGCTCGGCCTCGGCCGC
GCACTGGGTGAGACCATGGCGCTATACATGGTTGTTTCTCCATCCTCGGCGTTCCGCTTC
TCGCTTTTCGATGGCGGTACCACCTTCGCAACGGCCATCGCCAATGCCGCTCCAGAATTC
AACGACAACACCCGCGCAGGCGCGTAGATCTCCGCCGGCCTCGTGCTGTTCGCCCTTACC
TTCATCGTCAACGCTGGCGCTCGCGCCATGGTTAACCGCGGAAAG
>RXA00777-downstream
TAGAAGGGGACAAAATCATGACT
>RXA00828
GAGCACCAATTTGTGGCGCGCACTGTGCGTGATGAGCTAGAAATTGGTCCGAAAATCATG
AAAGTTGATGCAAGCGAGCGCATCGAGGAGTTGCTTGATCGGTTGCGCCTCCGCCACTTA
GAAAATGCTAATCCGTTTACCTTGAGTGGTGGAGAAAAGCGCCGCCTATCTGTGGCGACA
GCCTTGGTGGCAGCACCGAAACTTCTCATTTTGGATGAGCCTACGTTTGGCCAAGATCCC
GAGACCTTGACAGAGCTGGTGACGATGTTGCGTGAATTAACAGACAACGGAATCAGCATT
GTGTCAGTAACCCATGATCGTGATTTCATCGCAGCGCTGGGCGATCACCACATTGAGGTG
AGCGCGAAG
>RXA00828-downstream
TGAACCTGCTGATCAAAATTAAT
>RXA00832
ACACTGACAGCAGTGGTGTACGGGTTCTTCCTGTTTCGCCAAATGGGTGCGCAAGCTGGT
GAATTTCAAGAGGTCGAGGTCGCAGAAAAGGCAGACGACGCAGCAAAATGGGAGGTCCCA
TTTAGAGGCTTAATCTTGATTATCACTGTGCTCCCCATCGTGTTGCTGTCCCATGACATG
GCCACGGTGATGGATGAAGTCCTGGCAAGCCTTGGTGCACCCGTAGCAATGGCTGGATTA
ATTATTGCCACCATTGTCTTCTTGCCAGAGACCATCACCTCCTTGAAAGCTGCGTGGACA
GGAGAGATTCAGCGAGTAAGCAACCTGGCGCATGGAGCCGAAGTATCAACGGTGGGGCTG
ACAATCCCAGCTGTTCTAGTGATCGGCGTGATCACAGGTCAAGATGTAGTTTTGGGGGAG
ACCCCGATCAACTTGTTGCTGCTGGGAACCACCATTGCGGTGACAGCCATTGCGTTTAGC
TCCAAGAAAGTCAGTGCTGTGCATGGCTCGGTGCTGCTCATGCTTTTCGGTGTTTACATG
ATGAGCATGTTCGCC
>RXA00832-downstream
TGATTTAGGTAGCCTGGTGGGAA
>RXA00934
CCAAGTTTTTCCATGGCGGCGCTAGCGTTTGCGGAAGGCCCCATCGTTGCTACTTACCAC
GCCTCCAGTAGCGGATCGAAGCTGGTCAAGGCTTTCTTACCAGTGCTTTCGCCCATGCTG
GAGAAAGTGCGCGCAGGCATCGCCGTGTCTGAAATGGCTCGGCGCTGGCAGGTGGAGCAA
GTCGGCGGCGATCCCGTGCTGATCCGCAACGGGGTAGAGACCTCCATGTTCAAAGCCGCG
GGCCAAATCGAACCGAATGATCCTGTAGAGATCGTCTTTTTGGGTCGCCTCGATGAGTCC
CGCAAAGGCCTCGACATCCTCCTGCGCGCTCTGACCAGGCTGGATCGCCCGTTTACCTGC
ACCGTCATTGGCGGCGGCACCCCGCGAGAAGTCGCCGGCATCAACTTTGTGGGCCGCGTC
AGCGATGAGGAAAAGGCAGCAATCTTAGGTGGCGCAGACATCTATGTCGCACCCAACACC
GGCGGCGAAAGCTTCGGCATCGTGCTAGTTGAAGCGATGGCCGCGGGATGCGCTGTCGTC
GCCAGCGACCTAGAAGCGTTCTCCCTGGTCACCGATTCTGAAGCCGCACAGCCAGCGGGC
GTGCTATTTAAAACCGGCTCAGACGCCGACCTAGCCAAAAAACTTCAAGCGCTTATCGAC
GACCCCTCCTCCCGTTCCACGCTTATCGCCGCGGGGCTAAAGCGCGCAAACGCCTACGAC
TGGTCGACAGTATCCACCCAGGTCATGGCAGTCTATGAAACCATTGCGATCGACAAAGTG
AGGCTTGGA
>RXA00934-downstream
TGACCCTTGTTTACCTCCTCATC
>RXA00939
GGTGTCCTGCTCGGTGGAGTGACCATGTCGATTGGCATGCTCGTGCACGAGGCCTCCGTC
CTGGTGGTCATCGCGATTGCGATGCTCCTGCTGCGCCCGACCCTGAAGGAAGACAAGGAC
AAGGCAGACGTCAGTACTGCTGACGGCGCGAAGGAGAGGCTGAGCGCC
>RXA00939-downstream
TAACGACACAATCGCCACAGCCA
>RXA00942-upstream
CATTGACCGGCAGCTGGTCTGATTGACTGCACATCGTACATTGGACGGTGGACACGTTTC
AGAGTCCGCTGGATTTCATCACATCGGAAGGAAGAGAATT
>RXA00942
TTGAGTACCAAAAATTACCACGTCGAGGGTTTGACCTGCGCAAACGGTGTAGCTTCCGTA
GAGGATGAAATCGGCATTGTTGCGGGCACCCAGGGTGTGGATATTGATATTGAGACCGGC
CGCGTCACGGTGACTGGTGAAGGTTTCACTGACGAGGAAATCATTGAGGCTGTCGCGAAC
GCGGGCTACAAAGTTTCTGGGCGG
>RXA00942-downstream
TAGCACAATTACACATTCATCTC
>RXA00950-upstream
TCTCCGTACGCCCCACGAAGACACCTCGAAGTTCCCACGCGAAATGTTTCACGTGAAACA
TCTGCTGATTTTTGTCTCCAAGTGGAGTTGAATGAGAGTT
>RXA00950
ATGAACACTCCGGCAGTTCAGGTTCAAAATCTAAGTTTGAGTTTTGGGTCGTTCACAGCT
GTCAACGGCCTGAGCCTCACGGTGGAGCAGGGGAGCATTCACGGCTTCCTCGGCCCCAAC
GGTGCAGGAAAGTCAACAACCATCAGGGCACTCATTGGAGTGCTAAAACCCCAAACAGGT
TCAGTCGCTATTCTCGGCCAAGATCCTGTTGCTCACCCCGATGTCCTTCGAAGAGTTGGC
TACGTTCCAGGAGATGCCACACTGTGGGACAACCTCACTGGGGCGGAAGTTTTCAGGGCG
CTCGAATCACTCCGCAAGACTCCATCCAACCGAGCTCTAGAAAACGAGCTCATTGACGCC
TTCCAATTGGATCCCTCGAAGAAGATCCGCGAATACTCGACAGGTAACAGAAGGAAAGTC
AGTCTCATCGCGGCGCTCAGTCATGAGCCCGAGCTCCTCATCGTTGACGAGCCCACCGCA
GGCTTGGATCCCATCATGGAGCAAGTCTTTGTCACCTATGTCCGCAAGGCACGAACCAAC
GGCGCGTCCGTGTTACTCAGCAGCCACATTCTCAGTGAGGTGGAGCAGCTGTGTGATTAC
GTCACGGTCCTTAAAGAGGGGCGAGCAGTTGCATCTAATGAGGTGAGCTATCTGAGGAAG
ATCTCCGCTCACCGCATTACTGCCACGATTCCGGCGGTACCTCAACACCTTGCTGGCAGG
GGAGAAGTGGATTTCGATGCTGGCCATCTCAGCATCACCTGCGATGCCTCCGAGGTTCCC
GATATTTTGCGCATCATCATCGACGCTGGCGGCCAGGACATCATCAGCACCGCGGCGTCG
CTGGAGGAGATCTTCTTGCGTCACTATGGAGAAACGGTGAGTGGTTCAGAAAGCAAGGCA
TCACAA
>RXA00950-downstream
TGATCCGTCTTAATCTACGTCTT
>RXA00960
CTGAAAAACGATGTTGATGTCAACGTCGCAGGCTTTGTTGTCCCACTGTGCGCCACCATC
CACCTAGCTGGATCGATGATGAAGATCGGCCTCTTCACCTTCGCTGTTGTCTTCATGTAC
GACATGGAAGTAGGCGTCGGCCTCTCCATCGGATTCCTCCTCATGCTGGGCATCACCATG
ATCGCCGCACCAGGCGTTCCCGGCGGAGCCATCATGGCAGCAACCGGCATGCTGGCCTCC
ATGCTCGGATTCAACACCGAACAAGTCGCCCTCATGATCGCCGCTTACATCGCGATTGAC
TCCTTCGGCACCGCAGCAAACGTCACCGGCGACGGCGCAATCGCAGTCATCGTGAACAAA
TTCGCCAAGGGCCAGCTGCACACCACTTCCCCAGATGAAATCGAAGAAGACGACCGCGTT
GCCTTCGACATCACTCCATCGGATGTGGAACATCACAAG
>RXA00960-downstream
TAGAAACCCGCATTTTCTGTAGT
>RXA00980-upstream
GTTGATGGACAGGCAGGTTGCTGTTGGATCTGCTGAGTTACTTGATCATGAACCAGACTC
GACCAGGATGCTGGAGCTAAATGCCGAAGGAAAGACGGCG
>RXA00980
ATGTTTGTCGGAGTGAACGGACACGCCATTGGAATCGTGGCCGTCGCCGACGCCGTTCGT
TCAGATTCTGCCTCAGCAATCGAATCGGTGCATAAGGCGGGCATTCAAGTTGTCATGGCG
ACTGGCGACGCTCACCGCGTTGCACAAAACGTGGCCTCCAAGCTGGGAGTGGATGAAGTC
TACTCAGAGCTACTCCCTGAACAGAAATTAGAACTGGTGCGTGATCTGCAAGCTGGCGGC
AAAACGGTCGCGATGGTGGGTGACGGAGTCAACGACACCCCAGCATTGGCAGCTGCTGAT
ATCGGAGTAGCGATGGGGGTGGCAGGTTCCCCTGCAGCGATTGAAACCGCTGATATCGCA
CTCATGGCGGATCGTCTCCGACGGCTGGCACATGCAGTGACCTTGGCAAAACGCACCGTA
AGAACCATGCGCATCAATATTCTGATTGCGTTGGCTACCGTGATGGTGTTACTAGCTGGC
GTCCTATTTGGCGGAGTTACCATGTCGGTTGGCATGCTCGTTCACGAAGCAAGCGTGCTG
CTTGTTATCAGCATCGCCATGCTGTTGCTGCGTCCAACACTTAAAGAAGATGCTGCGGAA
GCAAGTGATATTAAACGCTCGGAAATACAACAGATCGCA
>RXA00980-downstream
TAACCAATGGCTGGGTACTGATG
>RXA01000
ATGTTGGCTGCCCGCGGGGTGGGACCTTATTGGCTGCGTACCGTTTTACGGTTCGTGTTC
GCGGTGATTCGTGCGTTCCCCGAAGTGGTTATCGCAATTATTTTGCTAACTGTCACCGGC
CTAACTCCTTTTACTGGTGCGCTCGCATTGGGTATCTCCGGTATTGGACAACAGGCAAAG
TGGACCTATGAAGCCATTGAGTCCACTCCCACCGGCCCGTCAGAGGCAGTGCGTGCAGCG
GGTGGAACTACGCCGGAGGTTCTGCGGTGGGCGTTGTGGCCACAGGTTGCGCCATCCATT
GCATCTTTTGCCCTGTACCGCTTTGAGATCAACATCCGTACCTCTGCGGTATTGGGGATC
GTTGGTGCAGGTGGTATCGGTAGTATGCTTGCCAATTACACCAACTACAGGCAGTGGGAC
ACCGTGGGCATGCTGCTCATCGTCGTGGTTGTCGCAACGATGATCGTCGATCTCATCTCC
GGCACCATCCGCCGCCGCATCATGAAGGGGGCTAGTGACCGTGTCGTGGCACCAAGCAAC
>RXA01000-downstream
TGACGCTCCACCAAGCATCCGCA
>RXA01002
CCCACGGAGCACGACAAGCAGATTGCTTTTCACGCGTTGGAGTCCGTGGGCATTTTGGAC
AAAGTGTGGACCCGAGCTGGTGCTTTGTCGGGTGGACAGAAACAGCGCGTTGCTATTGCG
CGCGCCTTATCGCAAGATCCGTCTGTCATGCTGGCAGATGAGCCTGTGGCAAGCCTTGAT
CCGCGAACCGCGCATTCCGTGATGCGCGATCTAGAAAACATCAACAACGTGGAAGGCCTC
ACCGTGTTGGTGAACTTGCACTTGATTGATTTGGCTCGTCAATACACCACAAGGCTTGTG
GGTTTGCGTGCCGGCAAGCTGGTCTATGACGGTCCTATCTCTGAGGCCACCGATAAAGAC
TTTGAAGCTATCTATGGTCGCCCCATCCAGGCTAAAGACCTGCTAGGTGATCGCGCA
>RXA01002-downstream
TGACCACGCCTTCTTCTACACTT
>RXA01003-upstream
AGCTGGTCTATGACGGTCCTATCTCTGAGGCCACCGATAAAGACTTTGAAGCTATCTATG
GTCGCCCCATCCAGGCTAAAGACCTGCTAGGTGATCGCGC
>RXA01003
ATGACCACGCCTTCTTCTACACTTATCCCACAAAAGCCTCGGGCTGGGGTAAAGACCTAT
CTCATCATCGGCGCCATCGTTGTCTTCACCGTGGCAACAGCAACCCCAGCGCTAGGTGGC
ATTGAGCTTGATTTCGCTTCCATTGCTGCGAATTGGCGCAATGGTGCCAACAAACTCCTG
CAAATGCTGCAGCCCAACTTTGCGTTCTTGCCTCGTACGTGGCTTCCCATGTTGGAAACC
CTGCAGATGGCGCTTGTTGGAGCTGTCTTGTCTGCTGCCGTATCGGTGCCTTTGACGTTG
TGGGCAGCGCAGGCAACCAACACCAGTGCGATTGGTCGTGGCATTGTCCGCACCATCATT
AACGTGGTGCGCTCTGTCCCCGACTTGGTGTATGCCACCATCTTGGTCGCCATGGTTGGT
GTCGGCGCATTACCTGGCATTTTGACGCTGTTTCTGTTCAACCTGGGCATCGTGGTCAAG
CTTGTCTCTGAGGCCATTGATTCCACTGAGCATCCCTATATGGAAGCAGGACGCGCAGCA
GGTGGATGACAGTTCCAAATCAACCGAGTGTCCGCGCTTCCTGAAGTCATGCCGCTCTTT
GCCAACGAATGGCTCTACACCCTAGAGCTGAATGTACGCATCTCCGCCATCCTTGGCATC
GTGGGCGCAGGTGGCATCGGCAGGCTGCTTGATGAACGCCGAGCTTTCTATGCCTACGCG
GATGTTTCCGTGATCATTCTGGAAATCCTCATCGTGGTGATTGTCATTGAAGTAATCTCC
AACGCACTTCGAAAGAGGCTGGTA
>RXA01003-downstream
TGAGCACCTTAACCTCTCACCGC
>RXA01006-upstream
GCCTTACGTGAAGGGCTTTAGCCCCGAAGTGATCGGCCGCCCCAGCTTCTATGAGACCTA
CATTGACCATTCCAGCGACCATTCCAGTGAGGAGGACTAA
>RXA01006
ATGACTACCTCGCAGATTCTGCGCCGCATCGGCCAAGCCGTCTTGGTCTTGTTGGTCACC
TTTACCTTGGCGTTCATCATGCTTTCCGCCCTCCCTGGCGATGCTGTGTCCGCCCGCTAT
TCCAGCCCTGATTTGGGTCTGTCACCTGAGCAGATCGCACAGATCCGTGAATCCTATGGT
GGCGATGAATCCCTGATCGCTCAGTACTTCTCCACCTTGGGTGGCTTCCTTGTAGGTAAC
TTCGGTTACTCCGTACAAACCGGAACTGCCGTGGCAACCCAGCTGGCAGAAGCCCTACCA
GGCACCTTGACCTTGGCTATTTTGGCATTCTTGCTCGCAGCCATTTTGGCACTGGTTATT
TCCATTCTTGCCACCATGGATCGCTTTGCATGGATCAAGGCCATCTTCCAGGCTCTGCCT
CCATTCTTTGTGTCCCTTCCAAGTTTCTGGTTGGGCATCATCTTGATCCAGATCGTGTCC
TTCCGCCTTGGTTGGGTCCCCGTTATTGGGACCACCCCGGCACAAGGACTGATCCTGCCG
ACCATCACCTTGTCCATCCCAATTACCGCTCCGCTTGCACAGGTGCTCATCCGCTCGATT
GAAGAGGTCAAGGCACAACCGTTCATCGCGGCTGTTCGTGCTCGCGGTGCGGGTGAAATG
TGGATCTTCTTCCGCAACATCATTCGCAACGCCCTTTTGCCAACCCTGACGATTGCCGGC
ATCTTGTTTGGTGAACTAGTCGGTGGGGCCGTGGTCACCGAGGCAGTGTTCGGCCGCGCT
GGACTTGGCCAAATGACCGTCAACGCAGTGGCCAAGCGCGATATGCCAGTGATGCTTGCC
ATCGTGGTGATCGCAGCT
>RXA01012-upstream
GACCTCATGGTGGCTGACTGTGCTGCCTGGTTTTGTCATCATCGCCGTGGTTATGTCTGC
CAACTACCTAAGCCGCATCATTCAGAAGGAGGCATAGAAA
>RXA01012
ATGACTACTCCCTTGTTAGAGATCAACGATCTGGTTGTCTCCTATCAAACTGCTAAAGGT
TTGGTGCATGCTGTCAACAATGTCAGCCTGGAGGTGCACCCTGGCCAAATCACCGCGATT
GTTGGTGAGTCCGGTTCTGGTAAGTCCACCACCGCTCAGGCCGTGATTGGTTTGCTGGCT
GATAATGCTGAAGTGGATTCTGGTCGGATTTCTTTCAACGGCCGTTCCCTTGTTGGCTTG
AACGCACGTGAGTGGAAAAACGTTCGCGGTACCAAAATTGGTTTGATTCCGCAGGACCCC
AACAACTCTCTGAACCCGGTGAAAAGTATCGGCGCTTCAGTGGGGGAGGGCTTGGCTATC
CACAAGCGTGGAACGGCCGCCGAGCGCAAAAAGAAGGTCATTGAGCTTCTAGAGCGCGTG
GGTATTGATAACCCAGAGGTCCGCTATGAGCAGTACCCGCATGAGCTGTCTGGTGGCATG
AAGCAGCGCGCGTTGATTGCCGCTGCCATTGCACTTGAACCAGAGCTGATCATTGCCGAT
GAGCCCACATGTGCGCTGGATGTGACCGTGCAGAAAATTATTCTCGATCTGCTGGAAGAC
ATGCAGCGTGAATTGGGGATGGGTATTTTGTTCATTACTCACGATCTAGCCGTGGCAGGC
GATCGGGCGGATCGCATCGTCGTCATGCAAAAAGGCGAGGTGCGCGAAAGTGGTTACGCG
GCTTCGGTCTTGACCGACCCCCAGCATGAGTATTCCAAGAAGTTGCTTGCCGACGCGCCC
TCCCTCACCATCGGCGAGATCCCCACGCGAGTTCCGGCCGTAGATCCGGAGGTAGCGCAG
GCCAAAGGCCCGCTTCTGGTAGTGGATAAATTCCGCAAGGAACACCAACGAGGCAAAGAA
GGAGCATTTGTTGCCGCAAATGATATTTCCTTCGAAGTACTGCCTGGCACCACGCATGCC
ATCGTCGGTGAATCCGGTTCTGGTAAAACCACGCTTGGCCGCGCGATCGCGATGTTTAAT
ACGCCGACCTCTGGTTCCATTTCAGTAAGTGGCAAGGACATCACCAACCTGTCCAAGGCC
CAGCAGCGGGAACTGCGCCAGCAAATCCAGCTGGTGTACCAAAACCCGTATTCTTCCCTG
GATCCTCGCCAAACCATTGGCTCCACCATCGCGGAACCTCTGCGCAATTTCACCAAGGTG
AGCAAGCAGGAAGCCGACGAAAAGGTGGCACACTACCTGGAACTGGTGGCGCTTGACCCG
GCTCTTGCCACCCGTCGCCCACGTGAGCTCTCTGGTGGTCAGCGCCAGCGCGTCGCCATT
GCTCGTGCCATGATTTTGGAACCTGAATTGGTGGTTTTCGACGAAGCCGTATCCGCGTTG
GATGTGACTGTGCAGGCACAAATCCTGCGCCTGCTCGACGATCTGCAACGAGAGCTAGGC
TTGACTTACGTGTTTATTTCCCACGACCTGGCTGTGGTCCGTGAAATCTCTGACACTGTG
TCTGTGATGAGTCGCGGCAACCAGGTGGAACTTGGAAAAACCGGAGAAGTATTTAACAAC
CCGCAAAGCGATTTCACTCGCCGACTCATCGACGCGATCCCAGGATCGCGCTATCGTGGT
GGCGAACTCAATCTTGGACTA
>RXA01012-downstream
TAGGAGCAGATCTTAAAAATGTC
>RXA01013
TTGGGCAATCCTTGGACGAGGCCTGCTGCTGTTATTTCCATCGTGGTACTCGCCGTTGCG
GTGCTGATGGCACTTGTTCCTGGACTGTTTACCTGCCAGGATCCGTTCACTGGCGATGAT
GTGGCGCTGCTTGGGCCAAGTGGCACCCACTGGTTTGGTACCGATTCCGTGGGACGCGAT
CTCTACAGTCGTGTTGTTTACGGCGCGAGGGAAACCCTGCTCGGTGCACTGATCGCAGTG
CTGGTTGGTCTGATCGTGGGAACCCTGATCGGACTGCTCGCAGGTGCACAGCGCGGTTGG
GTTGACACTGTATTAATGCGTTTCGTGGATGTGCTGTTGTCCATCCCGGGACTGCTGCTC
AGCTTGACTGTCATTATCCTTTTGGGATTCGGCACCATGAACGCAGCGATCGCAGTCGGT
ATTACCTCTGTTGCCACCTTCGCGCGTCTGGCGCGTTCCCAGGTGATGACTGTTGCAGGT
TCGGATTTCGTGGAAGCTGCATACGGTTCCGGTGGCACCCAGGCGCAGGTGTTGTTCCGC
CACATTCTGCCTAACTCTCTGACCCCAGTGTTTGCTCTTGCAGCACTGCAGTTCGGTTCC
GCGATTTTGCAGCTGTCCGTGTTGGGCTTCTTGGGCTACGGCGCTCCGGCACCAACAGCA
GAGTGGGGTCTGCTGATCTCTGATGCCCGCGACTACATGGCGACCTCATGGTGGCTGACT
GTGCTGCGTGGTTTTGTCATCATCGCGGTGGTTATGTCTGCCAACTACCTAAGCCGCATC
ATTCAGAAGGAGGCA
>RXA01013-downstream
TAGAAAATGACTACTCCCTTGTT
>RXA01070-upstream
CAGCTTCGGTTAATTTGGTCACACTAATGCAATAAATTCCTGTCTACAGCGTTACAGTTA
ATGAATTCAATTCAACCGCTAAACGCAAGGAGTGCTACCC
>RXA01070
ATGGCTAACGCCACCGCACAGAAGGGCCGTTTCGGCCTTCCCGGCTGGATGACTGGCTTT
GGTGCCCAGGTTATCGCCGGCCTCATTCTTGGTCTTATTCTCGGCCTTGTCGCCGGAGGC
ATGGACAGCGGCGCTGCAGACGGTGAAGCAAGCTGGCTTACCGGTCTTCTTAGCGGCGTC
GGTTGTGCTTATGTTTCTCTACTTAAAGTTATGGTTCCACCACTGGTGTTCGCTGCAGTG
GTTACCAGTGTGGCAAAGTTGCGCGAGGTAGCTAACGCTGCTCGCCTGGGTGTTTCCACC
TTGGTGTGGTTCGCCATTACTGCATTCTTCTCTGTGCTCGCGGGTATCGCCGTAGCGCTG
ATTATGCAGCCTGGTGTTGGATCCACTGTCGACGCATCTAATGCTGCTGATCCTTCTCGC
GTGGGCAGCTGGCTGGGCTTTATCCAGTCCGTTATTCCATCAAACATTCTGGGACTTTCC
GGTTCTTACAGTGAGAACTCTGGTGTGAACCTGTCCTTCAACGTGCTGCAGATCCTGGTT
ATCTCCATTGCGATTGGTGTTGCAGCTCTGAAGGCTGGCAAGTCCGCCGAGCCTTTCTTG
AAGTTCACCGAGTCCTTCCTCAAGATCATCCAGATCGTGTTGTGGTGGATTATTCGCCTG
GCTCCAATTGGTTCCGCTGCGCTGATCGGTAATGCTGTTGGTACCTACGGTTGGTCTGCA
CTTGGATCCCTGGGCAAGTTTGTTCTTGCGATCTACGTTGGTCTGGCAATCGTCATGTTC
GTTATCTACCCAGTGGTGCTGAAGCTCAATGGAATTCCTGTTCTTGGATTCTTGAAGCGC
GTTTGGCCTGTCAGAAGCCTTGGCTTTGTTACCCGTTCCTCCATGGGCGTTATGCCAGTT
ACCCAGCGCGTTACTGAGCAGTCCTTGGGTGTTCCATCTGCGTACGCTTCCTTTGCTATC
CCACTGGGTGCGACCAGCAAGATGGACGGCTGCGCTGCTGTCTACCCAGCTGTTGCCGCT
ATCTTCGTGGCACAGTTCTACGGCATTGACTTGAGCATCATGGATTACGTACTGATCATG
ATCGTCTCTGTCCTGGGCTCTGCTGCAACTGCAGGCACCACTGGCGCAACCGTCATGCTG
ACCCTGAGCCTATCCACCTTGGGTGTGCCACTTGCTGGTGTTGGTCTGCTGCTGGCTATC
GAGCCAATCATCGACATGGGACGTACCGCAACCAACGTCACCGGTCAGGCACTGGTTCCT
GCGATCGTTGCTAAGCGCGAGGGCATTCTGGATCAGGATGTGTGGGATGCTGCTGAAAAG
GGTGGCGCTGCTATTGAAATGGCAACCGTCTCTGAGAAAGAAACTGAGCCTGCAGAGGTT
CGCTCC
>RXA01070-downstream
TAAGCTCTCTTGAGTACCTGAGA
>RXA01094-upstream
GTCAGCTGACCGGGGTAGCGGGCGGTGGGCGGACAAGTCTTGCTAGATTGAAGTGCATTA
CTTGTGGCCTGACTGTTAGGTTTACGTTGTTGTGGATGTC
>RXA01094
ATGACTTTGGCGACGATTCCCTCACCACCGCAGGGTGTGTGGTACTTGGGTCCCATTCCG
ATTAGGGCCTATGCGATGTGCATGATCGCTGGCATTATTGTTGCCATTTGGCTGACGAGA
AAGCGCTACGCCGCCCGCGGTGGAAACGCTGAAATCGTCGTTGATGCAGCGATCGTGGCA
GTTCCTGCCGGAATCATCGGTGGACGCATTTATCACGTCATTACCGACAACCAAAAGTAC
TTCTGGGATACCTGTAACCCCGTCGACGCCTTCAAAATCACGAACGGTGGTCTGGGCATC
TGGGGTGCAGTGATCCTCGGTGGCCTGGCAGTGGCCGTATTCTTCCGGTACAAAAAGCTT
GCTCTTGCACCTTTCGCAGATGCCGTGGCACCTGCAGTTATCCTGGCGCAGGGAATTGGT
GGTCTGGGCAACTGGTTTAACCAGGAGCTCTACGGTGCAGAAACTACCGTTCCATGGGCT
TTGGAAATCTACTATCGGGTAGATGAAAATGGAAAATTCGCACCGGTGACAGGAAGATCC
ACCGGTGAAGTAATGGCTACTGTTCATCCAACATTCCTCTATGAAGTGTTGTGGAACCTA
CTGATCTTCGCTTTGTTGATGTGGGCTGACAAGCGATTCAAGCTGGAACATGGCCGAGTA
TTTGCTCTCTACGTAGCTGGTTACACCTTGGGCGGTTTCTGGATTGAACAAATGCGCGTT
GATGAAGCCACGCTTATTGGCGGCATCCGAATGAACACGATCGTCTCCGCAGTAGTGTTT
GCCGGCGCGATCATCGTGTTGTTCCTGTTGAAGAAGGGTAGGGAAACTCCCGAAGAGGTA
GATCCGACTTTCGCAGCGTCTGTTGCAGCAGATGCTGTAGCTTCGCCAGATAGAAAACCC
TTGCCGAAAGCAGGGGAGGGCATTGATGGAGAAAGGCCCTCAACGCGA
>RXA01094-downstream
TAGGTTTCAACCATAGGCCTGAC
>RXA01135-upstream
CATTTACTAATCTCACAAGACATCGCCTAATGAATACAGACTAGCCTATTCAAATTCAAA
GAACACTCGGTATGGCACGTGATTTAAGGATGCTGCAATC
>RXA01135
GTGACACATATCCTCTTCGACAGCAGGCGTTTTCTGCAACTGGGCGCTTTTGCGTCCTTG
AGCACCGCATTGGCCGGAGCGGCCCGCTACGTGACGTCGACAAGCAATAATGAACCTGCG
GATAACACTCCCCTGACCATTGGCTACGTGCCTATTGCGGGCTCGGCGCCGATTGCTATC
GCAGATGCGCTAGGGCTGTTTAAGAAACACGGCGTGAATGTCACGTTGAAGAAGTACTCA
GGCTGGTCCGACCTGTGGACCGCCTATGCAACAGAGCAGCTTGATGTTGCGCAGATGCTG
TCGCCGATGACTGTGGCGATTAAT
>RXA01141
GTCAATTCAGCGGCGGATCTTAAAGGCATGGTGCTGGGAATTCCTTTTGAATATTCAGTC
CATGCGCTGCTCCTGCGCGATTATCTCGTCTCAAACGCAGTTGATCCCATCGCCGATCTT
GAGCTTCGCCTGGTCCGACCTGCCGATATGGTCGCACAATTGACAGTTGAGGGCATCGAT
GGATTCATTGGGCCTGGGCCGTTTAATGAACGCGCGATCAGCAATGGCTCCGGCCGGATT
TGGGTGCTGACCAAACAAGTGTGGGACAAACATCCATGCTGCGCCGTGGCGATGGCCAAA
GAGTGGAAAGCTGAACACCCCACGGCGGCTCAGGGTGTGCTTAATGCGCTGGAGGAAGCC
TCCGCAATTTTGAGCAATCCGGCACAATTTGATTGCTCGGCACGCACGCTGTCGCAGGAA
AAATACCTCAACCAGCCTGCCACGTTGCTGGATGGACCGTCG
>RXA01141-downstream
TAATCATCGGCATCACCGGCTTA
>RXA01142
ACCCGCACCCACCTCGAACAAGTAGGCCTCACCGACGCCGCCGAACGGCGCCCCGCCCGC
CTCTCCGGCGGCATGCAACAGCGAGTCGGCATCGCACGCGCCTTCGCCATCGACCCACCA
ATGATGCTTCTCGACGAACCCTTCGGAGCCCTCGACGCCCTCACCCGCCGCGAACTCCAG
CTCCAACTACTCAACATTTGGGAAGCCTCCCGCCGCACCGTCGTCATGGTCACCCACGAC
GTCGACGAGGCCATCCTGCTCTCCGACCGAGTTCTCGTGATGTCCAAGAGCCCCGAAGCC
ACCATCATCACCGATATTCCAGTGAATCTTCCCCGCCCCAGACACGAGCTGAGTGAAGAC
GCTTCTGTTGAAGCCGAGACCACAGCCCTGCGTAAGCGGATGCTGCATCTGCTGGAGCAC
>RXA01142-downstream
TAGTTTCTAACACGTCTTTTAAA
>RXA01164-upstream
GCCGATCGTGATTGATGAAGACGAGATCCAAGCGTGGACTTCTGATGTCAAACCTGAAGA
TTTCACCAAAGGTAAAGATGAATCCGACGGTGAGAAATAA
>RXA01164
GTGACAGTGTTTGTTCGGCTCGCCCTTGCTGCTGTGGGCGGGCTTTTTGTCTTTGGTTGC
AATGAACCGATCGGCTGGTTTGTCGGGGGAATTGTTGGCACTGCATTATTTTTTATCTCC
CTTGCGCCGTGGGATCTGGGAGTTCGCCAAAAGCGGCGGAAGAAGAATGAGCCAGTGCCA
TTTTTGCAACAGATGTCCACGGGCCGAACTGTTGTACAGGGCATGCTTTTAGGTTTTGTC
GATGGCCTGGTGACATATTTGCAGCTGTTGCCGTGGATCGGTGAGTTTGTTGGCTCACTG
CCTTATGTCGCGTTGTCAGTTGTCGAGGCGCTTTATTCCATTGCTCTTGGTGCTTTCGGC
GTGCTCATTGCGGGTTGGAGGGACTGGAAGGTTCTGCTGTTTCCGGCGATGTATGTGGCT
GTGGAGTATCTAACAAGCTCGTGGCCATTTGATGGATTCGCGTGGGTTCGCCTGGCATGG
GGTCAAATTAACGGTCCGTTGGCTAATCTCGGAGCGGTTGGTGGGGTAGGGTTTGTCACT
TTTTCCACGGTGCTGGCTGCCGTGGGTGTGGCGATGGTGATTATTTCCAAGAAGCGACTG
GCCGGCGCAATCATCACCGCGAGTGTGATTGCTATCGGCGGGGTGTCATCCCTGTACGTT
GACCGCAATGGCACGAGCGATGAAAGCATGGAAGTAGCCGCAATTCAGGGCAATGTGCCT
GGGATGGGATTGGACTTCAATGCACAGCGCCGCGCGGTGCTGGCGAATCACGCACGGGAA
ACCCTGAAGCTGGATGAACAAGTGGATTTGGTGATCTGGCCGGAGAATTCCTCAGACGTC
AACCCATTTTCCGATGCACAAGCAAGAGCCATTATCGATGGAGCAGTGGAAGATGTTCAG
GCACCTATTTTGGTGGGGACGATCACCGTCGATGAGGTTGGTCCACGCAACACCATGCAG
GTATTTGATCCTGTTGAAGGTGCGGCGGAGTACCACAATAAGAAGTTCTTGCAGCCGTTT
GGTGAATACATGCGGTTTCGCGAATTCCTGAGAATTTTCTGGCCCTACGTTGATTCCGCT
GGAAACTTCCAGCGCGGTGATGGCACCGGCGTAGTGGAGATGAATGCTGCGAACTTAGGG
CGCGCTGTGACAGTGGGCGTGATGACGTGTTACGAGGTCATCTTCGACCGTGCTGGCCGC
GACGCCATCGCCAATGGGGCTGAATTTTTGACCACGCCCACGAACAACGCCACCTTCGGA
TTCACGGACATGACGTATCAGCAATTAGCAATGAGCAGGATGCGTGCCATCGAATTTGAT
AGGGCGGTGGTTGTTGCAGCTACATCGGGTGTTTCGGCTATCGTCAACCCTGATGGAAGC
ATTTCCCAAAACACCGGAATTTTTGAGGCCGCCACCTTGACGGAATCCATTCCACTCAAG
GACACTGTCACCATCGCAGCGCGGGTTGGTTTCTATGTTGAATTACTGTTGGTTATCATT
GGTGTATTAGCTGGACTATTCGCCATTCGAATGAATAGCCGTTCAAAGTCTGCGAAAGGT
TCCGCTCGGCCCGCA
>RXA01168
CGCACCGCAACCCCTGACGTTCACGTACTCATCGTGGACGACAACAGCCCAGACGGCACC
GGCGAGCGCGCAGACAAGCTTGCTGCTGACGACGACCACATTTTTGTCCTCCACCGCGAA
GGCAAAGGCGGCCTGTGCGCAGAGTACATGGCTGGCTTCCAGTGGGGCCTGGAGCGCGAC
TACCAGGTCCTGTGCGAAATGGACGCCGACGGCTCCCACGCACCAGAACAGCTGCACCTG
CTGCTCGCTGAGATCACCAATGGCGCTGACCTGGTCATCGGCTCGCGCTACGTGCCAGGC
GGCCGCGTAGTCAACTGGCCCAAGAACCGTTGGCTCTTGTCCAAGGGCGGCAACGTCTAC
ATCAGCGTCGCGCTCGGCGCCGGCTTGACCGATATGACCGCAGGGTACCGCGCTTTTCGA
GGTGAAGTGCTAGAAGCAGTGCCGCTTGATGAGCTCTCCAACGCTGGGTACATTTTCCAA
GTTGAGATTGCCTACCGTGGAGTTGAAGCCGGATTCGATGTTCGTGAAGTTCCCATCACT
TTCACCGAGCGTGAGATCGGCGAATCCAAGCTGGACGGCAGCTTTGTCAAGGATTCCGTG
CTCGAGGTAACCAAGTGGGGCGTCAAGCACCGCGGTGGCCAGGCCAAGGAACTGTCCAAG
GAAATGGTCGGGCTGCTGAACTATGAGTGGAAGCACTTCAAAAAGCGGAACACCTGGCTC
>RXA01168-downstream
TAAACTGCTTGCCGGTTAGTGAA
>RXA01185-upstream
TCAGCTGGTAGCGTCGCATGTATTATTGGTGCTTTTCACTAATAGCAATGCACTAACGCA
CATAGCCGCCGACACGTGAATCGAAAGAAGTTCATCTCCG
>RXA01185
ATGACTGATCCTGAAAACTCGCAAGGAACCCCACAGATTTGTCCGACTGATCCGACTACG
CAAGCATTAGGAGTTCGGGGCTTAACCAAGTCCTATGGTGATGCAACAGTAGTGAACAAT
ATCAATCTGGACATCCCCAAAGGAGCCATTTACGGCATCGTTGGACCTAATGGTGCAGGT
AAAACCAGGATGCTGTCCATGGCAACGGGTTTACTGAGGCCGAATAAAGGCAGCGCGTGG
ATTTCGGGTTTCAATGTGTGGGAAGAGCCAAACGATGCAAAACGAAGCATGGGATTGTTG
GCAGATGGCTTGCCCATCTTTGATCGCTTGACTGGCAAAGAACTGCTCACATATGTCGGG
GCATTGCGTGAGTTGGATGAAGGCATTGTTGATCAACGTAGTGAGGAATTGCTGGAGGCG
CTCGGGCTTAAAGAAGCAGCGGGCAAGAGAGTCGTCGACTATTGCGCCGGCATGACGAAG
AAGATTCTTTTGGCCCAGGCCGTCATTCACAATCCGAAAGTGCTCATCCTTGATGAACCT
TTGGAAGCGGTTGATCCGGTGTCTGGTCGTTTGATTCAGCAGATTTTGAAGAAGTTTGGG
CAAACGGGTGGAACCGTCGTTTTGAGTTCGCATGTGATGGAATTGGTTGAGGGGTTGTGG
GATCACGTTGCCATCATCAACAGGGGAGTGGTGGAGATTGCCGGACATGTGAATGAGGTT
CGTCGGGGCAGATCTTACCGGATGTCTTCGTTAATGCGGTTGAAGGCGCTGCTGTTCAAG
AGGGGTCACTATCTTGGTTGGGTGCGTCCGAAGGCCATAGCGAAGGCCAAAATCAGAACG
AGGATCGGGCTGAGTAAA
>RXA01185-downstream
TGACTAAAACACTTCTGAAACTA
>RXA01188-upstream
AAACAGTTAGAAGCAGCTAAACAGTTTTTCCGATAACTGTTGTCATTTTGTGACCGCCTG
GCTTTGTAGTTTTCGCTCCGGTGTCCAAGATGAATATGAC
>RXA01188
ATGATGAATGGCGTGGTAGAGCCTCAGGAACATCTCGATGCAACGTTGATTGCTGCAGAC
TTCCACGGCAACCCCGAAAACTCTGGTGACCGCAAAGAGCGCCTGAATTTTCAAGGTTGG
AAGTATGCCCTTAATCGCACGGTCAGGGATGTTTTTCCAGATGGCCTGCTCGATTTGGCG
GCCTTGTTGACGTTCTTTTCCATTCTGTCGATCGCCCCTGCAGTGCTGCTGGGCTATTGG
GTGATCACGATTTTTCTGGCCAGTGACTCCACCGAAATCCTCAACCTTGTCCGCGATGAG
GTAAATCAGTACGTTCCGGAAGATCAATGGCATGTTGTCAACGGCGTGATTGATTCGATC
GCAGGCTCGGCAGCTGCAGGTCAGGTCGGTGTCGCGGTCGGTGTGATCACGGCATTGTGG
ACATCTTCGGCATATGTGCGCGCTTTTTCCAGATGTGCCAACGCTGTTTATGGCCGAAGC
GAAGGCCGCACATTGATCAAACGCTGGGCAATGCTGCTTTTCCTCAACCTTGCTTTGGTG
CTTGGAATCATCATCATTTTGGTCTCCTGGGTGCTCAACGAGACCTTGGTGATGGGAATT
TTCGCCCGCATCGCGGAACCACTTCATCTCACGAATGTGCTCAGCTTCCTCACGGACCGG
TTCATGCCGATCTGGATCTGGGTGCGGTTCCGAGTGATTGTGGGGGTGCTCATCATGTTC
GTGGCCACGCTGTATTACTGGGCCCCGAACGCCCGCCCGTGGAAGTTTCGCTGGCTCAGC
CTCGGATCATTCTTGGCGATCGTTGGCATCCTGCTCGCAGGCGTGGGCTTGAATTTCTAC
TTCACGCTGTTCGCCGCTTTTAGTTCCTACGGCGCGGTGGGTTCGCTGCTCGCGGTTTTT
ATTGCGCTGTGGGTGTTCAACATTTGCTTAATCATCGGCCTGAAAATCGACGTGGAGATC
AGCCGCGCCAAGCAACTGCAGGCAGGAATGCCGGCGGAGGATTACAGTTTAGTGCCACCA
CGCTCTATCGAGAAGGTGGCGAAAATGAAGCAGCGCCAGCAGCGCTTGATGGATCAGGCT
GCGGCGATCCGGGAGGAAAGCAAT
>RXA01188-downstream
TAAAAAATTGCTTATCGACGTCC
>RXA01245
GCCTCCTGGGTCACCACGCTGGGGCTGGGCGGGTTCCACCTAGATTTCTGGTGGGAACTG
GCCCTGCTGGTGACGATAATGCTGTTGGGCCACTGGCTGGAGATGCGCGCTCTTGGTGCA
GCCTCCTCCGCGCTTGAGGCGCTGGCAGCGCTCCTGCCCGATGAGGCCGAGAAGGTCGTG
GACGGGACCACCCGCACCGTAGCGATCTCAGAGCTGGGGGTCGACGATGTGGTGCTGGTG
GGAGCAGGTGCCGGCGTCCCGGCCGACGGGACCATGATGGACGGAGCGGCCGAATTCGAT
GAGGCCATGATCACCGGCGAATCCCGACCCGTCTACCGGGATACCGGTGAGACCGTGGTG
GCCGGCACCGTGGGCACCGACAACACCGTCCGTATCCGGGTGGAGGCCACCGGTGGGGAC
ACCGCCCTGGCAGGCATCCAGCGCATGGTCGCCGACGCCCAGGCCTCCTCCTCCCGGGCC
CAGGCCCTGGCCGATCGAGCCGCAGCCTTACTGTTCTGGTTCGCCCTGATCACGGCCCTG
ATCACCGCCGTGGTCTGGACCATCATCGGCAGCCCCGACGATGCCGTGGTCCGCGCGGTG
ACCGTGCTGATCATCGCCTGCCCGCACGCCCTGGGCCTGGCCATCCCGCTGGTCATCGCG
ATCTCCTCCGAGCGCGCCGCGAAATCCGGGGTGCTCATCAAGGACCGCATGGCACTCGAG
CACATGCGCACCATCGACGTCGTCTTGTTCGATAAGACCGGCACCCTGACCGAAGGCGCA
CACGCCGTCACCGGCGTGGCTCCGGCCACGGGTATCGCCGAGGGTGAGCTGCTGGCCCTG
GCCGCCGCCGCTGAGGCCGATAGTGAGCACCCCGTGGCCCGCGCGATCGTGACTGCCGCG
GCCGCACACCCGGAGGCCTCGCAGCGTCAGCTGCGCGCAACCGGTTTCACCGCCGCCTCC
GGCCGCGGGATCCGGGCCACCGTGGACGGTGCCGAAATCCTCGTGGGCGGGCCGAACATG
CTACGCGAGTTCAATCTGACCACCCCGGGTGAGCTCGCCGACATCACCGGTTCCTGGGCA
GAGCGAGGTGCCGGAGTGGTACATGTCGTCCGCGACGGTGAGATCATCGGTGCGGTGGCA
GTGGAGGACAAAATCCGCCCCGAATCCCGCGCGGCGGTACGCGCCCTGCAGGCCCGCGGG
GTGAAGGTGGCGATGATCACCGGTGACGCCACCCAGGTCGCCCAGGCAGTGGGCAAGGAT
CTGGGGATCGATGAGGTCTTCGCCGAGGTTCTGCCGCAGGACAAGGACACCAAGGTCACC
CAGCTGCAGGAGCGCGGTCTGAGCGTGGCCATGGTCGGCGACGGTGTCAATGACGCCCCG
GCCCTGGCCCGGGCCGAGGTCGGTATTGCGATTGGCGCGGGTACAGATGTGGCGATGGAG
TCCGCCGGGGTGGTCCTGGCCAGTGATGATCCCCGGGCGGTGCTGTCGATGATCGAGCTC
TCCCATGCCAGCTACCGGAAGATGGTCCAGAAGCTGGTCTGGGCGACCGGGTACAAGATC
GTGGCCGTTCCGCTGGCGGCCGGTGTGCTCGGCCCTATCGGTGTGCTGCTTCCGCCGGCG
GCGGCCGCCATCTTGATGTCCCTGTCCACGATCATGGTCGCCCTCAACGCCCAGGTGCTA
CGCCGGATCGACCTGGACCCGGCTCACCTAGCTCCGACCGACGGGAAGGAGGAGAAGGCT
GCTGTGAGCTCTGCAGCCCCCGTCCGC
>RXA01245-downstream
TGACTTTCAATGCTTCATGGACT
>RXA01247-upstream
TCCGATGACCACCCCGACTTCCCCCTTGCTGCCGCTGGCCTCCGACGGTTGTGGATGCTG
CGCGCGCTCTACACCGTCCGCGAGCGTCTCCGCCTCGGCC
>RXA01247
GTGGCCGCGGCAACCGACGCAACACCTGAAGGTCCCACCACCTACCAGGTCACAGGCATG
ACCTGCGGACACTGCGGCGACAACGTCACCGAGGCGGTGAGCGCTCTGCCCCAGGTCGAC
GACGTCCAGGTCGACCTCATCGCCGGTGGGGTCTCCATCGTCACGGTCACGGGTTCCGTG
CCCCTGGAAACCGTCCACCGGGCAATTGAGGAGACCGGCTACACCGTCTTGTCC
>RXA01247-downstream
TGATCGATTCACCCATGATCTCG
>RXA01285
CCACAGACCTCCATCGGCCCAGAAGGCATCCGGGTTTACGATCTCATGGCGCGCGGGCGC
GCTCCCTACCAAAGCCTCATACAACAATGGCGCACCTCCGACGAAGAGGCCGTCGCGCAA
GCGCTCGCCTCCACGAATCTCACCGAACTTGCAGCTCGCCTCGTCGATGAACTCTCCGGT
GGCCAGCGCCAACGAGTGTGGGTGGCCATGTTGCTCGCCCAGCAAACACCGATCATGCTT
CTCGACGAGCCCACCACGTTCCTCGACATCGCCCACCAATACGAACTCTTGGAATTGCTG
CGCGCATTCAACGAGGCCGGGAAAACTGTGGTCACTGTGCTTCACGATCTCAACCAAGCC
GCCCGCTACGCCGACCACCTCATCGTGATGAAAGATGGGCACGTACATGCCACGGGCACA
CGGGAGGAAGTCTTAACTGCGGAGATGGTTCAAGGAGTTTTTGGCCTGCCCTGCATCATC
TCCCCAGACCCCGTCACAGGAACCCCCACCGTCGTTCCCCTCAGTCGGTCTCGCGCAGGA
GCT
>RXA01285-downstream
TAAGTAGGTACGCGTCCAACGGA
>RXA01289-upstream
CTCACCTAAGATGTTGTAAGCGTTTTAGTTTCAGCTAGTTTTAAGGAGTTTCGATGTCTG
ATACTTTTCTTTCCCCCTCGATCTTTAGGAGTCACGCGAT
>RXA01289
ATGACGGCGGTGGCGGTAGAGAAGCAGAAGGAGACGTCGATAAGCAAAAACCTCGGCAGG
CGCCGAGCGCTGGGCATTCTCGGAATCGTCGTGGCACTGGGTGCGCTTATTGTTTTAAGT
ATTGCTGTGGGTGCGAACCCACTTTCTTTTAGCTCCGTATGGCAGGGTTTTACCGCACAC
GACAGCTCTGAGGCGTCGATTATCGTGTGGTCAATGCGTATTCCGCGCACGCTGGTGGGC
ATCGTGACTGGCGCTGCTTTTGGTGTGGCGGGTGCTTTAATTCAAGCGCTGACGCGCAAC
CCGCTTGCCGATCCCGGAATTTTGGGAGTTAACGCGGGTGCAGGTTTCGCAGTGACCGTA
GGTGTCGGATTTTTCGGACTCAGCAGCGTGACGGGCTACATCTGGTTGGCATTCCTGGGC
GCTGCCGCCGCTACCCTGCTGGTGTATTTCATTGGTGCGAGCACCAGCGGCAGCGTTAAT
CCTGTTGCTCTGGTCCTCGCCGGCGTTGCTCTGGCCGCCGTGCTTGGTGGCGTCACGAGC
TTCCTCACACTGATTGATGCTGAGACTTTTGAAAGCATCCGCAATTGGAATCTTGGTTCT
GTTGCACGCACCGACCTCAGCGACACCATGACCGTATTGCCATTCCTGGCAGTCGGACTG
GCCATCGGGCTCCTGCTGTCGGGAGCACTGAACTCCATTGCGCTTGGCGATGACCTTGCT
GCATGCCTGGGCACCAAAGTGATGGGCACCCGCGTGCTCGGCATGATTTCAGTCACCTTG
TTGGCCGGCGGCGCGAGCGCCCTTAGTGGTGGTATCGGCTTCGTAGGCCTTATGGTTCCC
CACGTTGTGCGCTGGGTAGTTGGCCGCGATCAACGATGGATCATCACCTTCAGCGCCCTG
TGCGCCCCTGTTCTTGTAGTCGGCGCAGACATTTTGGGACGCATCATCGCCCGCCGCGGC
GAAATTGAAGTAGGCATTGTTACCGCAGTCATCGGCGCACCTGTCCTGATCGGACTAGTT
CGACGGAGGAAAGCCAGTGGTCTT
>RXA01289-downstream
TAATATCAAATCTAGAACTGATG
>RXA01290-upstream
GGACGCATCATCGCCCGCCCCGGCGAAATTGAAGTAGGCATTGTTACCGCAGTCATCGGC
GCACCTGTCCTGATGGCACTAGTTCGACGGAGGAAAGCCA
>RXA01290
GTGGTCTTTAATATCAAATCTAGAACTGATGAAACTCGTGTTGCTGCGTCTGAGGCGGTG
GAATCCACTAGACCTGTGTCTGAAGCTTCGACAAGCCCTGCGCTTAACCCCGGCTACCAC
GCAGTTTCAGTGCAGAGGCGCCGGTTCTCTTTCCGCATCCCAGCCCGCCTCATGGTGGTT
AGCCTTATCCTTTTCGCGATCGCGCTATGCAGCGCCAGATGGGCTATCACGATGGGCGAT
TACCCACTGTCTTTGGGGCAGGTGATTAATGGACTTGCTGGCACCGGCGAGAAATTCCAG
TTGTTGGTGGTGCGGGAATGGCGTGTACCTGTAGCCATTGCTGCTGTTGTCTTCGGCGCG
CTGCTTGGCATAGGTGGAGCGATTTTCCAGTCGATTACTCGAAACCCGTTGGGTTCACGT
GAGGTGATTGGTTTCGATGCAGGTTCTTACACGGCGGTGGTTCTTGTCATTTTGGTCCTG
GGCAACACTCACTACTGGAGCATCGCTTTCGCTGCCATCGTCGGTGGCATTGTTACGGCC
TTTGCCGTGTATGTCCTGGCGTGGCGTAAAGGTGTGCAAGGTTTCCGCTTGATCATCGTG
GGCATCGGTGTCTCGGCCATGCTCAGTTCGGTTAACGCGTATCTAATGACCCGCGGCGAT
GTGGAAGACGCCATGGTTGTGGGCTTCTGGAGTGCCGGTTCCATCAACCGCATTACCTGG
CAATCTCTGCTCCCCTCTCTGGTGATCGCTGCTGTCATCATCGTGGCCGCCATTGTGCTG
GCAAGGTCACTGCGTTTCATGGAAATGGGCGATGACGTAGCCACCACCCTCGGTGTGAAA
ACAAACTCCACCCGCTTGGCACTCATCGTTGTCGGCGTTGCTACCTCCGCGTTGGTTACA
GCAGCTGCCGGACCGATCTCCTTCATCGCGTTGGTTGCCCCACAGCTGGCACGTCGCCTC
ACTAAAACCCCTGGTGTCAGCCTGGTTGCTGCCGCTGCAATGGGTTCCGCACTGCTCAGC
TGCGCTCACCTCCTTTCCCTGATTATCAGCTCCTTCTACCGCACCATCCCGGTTGGCCTG
TTGACTGTATCCATCGGTGGTTGCTAGATGATGTGGCTTCTGCTGCGCGAAACCCGCCGC
CAATACCGCACCGGCACCATCCGA
>RXA01290-downstream
TAGTTCTTTTAAGGATCCCTCAT
>RXA01297-upstream
TCCTGTTGCTGCTGATTATCACTGTTATCCAGGTTCGATACATGGATAAGGAGAACAAGC
AGAAATGATCTCGACTGATAGAAACGTTTTGGTCAAAATC
>RXA01297
ATGGGCTATGTCGGCATGGTTCTTGCCATCTTGTTCATTGGCCTTCCGCTGGTATTTATT
GTGCTGACTAGCTTCAAGCAGCAGTCAGAGATTTACACCCAGCCGGTCACGTGGTTGCCT
TCGGAATTTAATTTCGATAACTATGCAAATGTTTTCGAGCGGGTTCCGTTCCTGAAGTAC
TTCCGCAACTCGATCATCATCACGGTTATTTTGTGTCTGGTGAAGATTATCTTGGGTGTG
ATCTCTGCATATGCGTTGTCGATTTTGCGCTTCCCGGGTCGAAACCTTGTGTTCTTGCTG
GTTATCTCCGCGCTGATGGTGCCTTCCGAAGTGACTGTTATTTCCAACTATGCGTTGGTC
AGTCAGCTTGGTTGGCGCGATACCTACCAGGGCATCATCGTTCCGCTAGCGGGTATTGCT
TTCGGAACGTTCCTCATGCGTAACCACTTCATGTCTATTCCTTCTGAGCTCATTGAAGCT
GCGCGAATGGATCACTGTGGACACTTCAGGTTGCTCTGGAAGGTTTTGCTTCCAATCTCT
ATGCCTACGTTGGTGGCGTTCTCCATGATCACCGTGGTGAATGAATGGAACCAATACCTG
TGGCCTTTCCTGATGGCAGAAACCGATAATTCAGCAACTCTGCCCATTGGTTTGACCATG
CTTCAAAACAATGAGGGTGTCTCCAACTGGGGACCTGTCATGGCCGCAACGATCATGACC
ATGTTGCCTGTGCTTGTGATGTTCTTGGCACTGCAGGAGTACATGATCAAGGGACTTATG
TCCGGCGGCGTCAAGGGC
>RXA01297-downstream
TAAAAACTTCTCGCTAAAAACTT
>RXA01298
TTCGTGTGGAAGAACTTGGGCTACTCCTTTGTTATCTACGTGGCTGCATTGCAGGGGCTA
AACAAGGATTTGTCTGAGGCCGCACCGGTGGATGGCGCGAGCGCGTGGACACGTTTTTGG
AAGGTTAGTCTTCGGCAGCTTCGCCCAACCACGTTCTTCCTTTCTATTACTGTCACGCTG
AACTCGGTTCAGGTCTTCGACATGATTCACACCATGACTCGTGGTGGCCCCTTGGGTAAC
GGTACGACCACGTTGGTTTACGAGGTGTACACCGAGACTTTCACGAACTATCGGGGGGGA
TATGGTGCAACAATCGCAACGATTTTGTTCCTGTTGCTGCTGATTATGACTGTTATCCAG
GTTCGATACATGGATAAGGAGAACAAGCAGAAA
>RXA01298-downstream
TGATCTCGACTGATAGAAACGTT
>RXA01303-upstream
AACATGCGGGCGCAGGTCAGAGCTGTTATCTTAGTACTTATCACAGCCATAGGGCGGGCT
TGACGGAAAGCCTTTCCGCGTAACGATGAAGAGGGATCAC
>RXA01303
GTGACACAACTCAACACCAAAGGCGTTGTTCTGCAAGGGTGGGATCCAGAAGATCCTGAA
CATTGGGACTCGAAAATTGCATGGCGAACCCTGTGGATTACGACCTTGTCCATGATTATT
GGGTTCTGCGTGTGGTATTTGGTTTCTGCCATCGGTCCGCTACTCAATCGAATTGGATTT
GATCTCTCAGCAGGTCAGCTTTATTGGCTCGCATCTATGCCCGGTTTGGCCGGCGGATTA
ATCCGATTGATTTACATGTTCCTTCCACCGATTCTTGGAACCCGCAAATTGGTGGGAATT
TCCTCCGGTCTATTTTTGATCCCCATGTTTGGGTGGTTCCTGGCTGTCCAAGATTCAAGC
AGTCCCTAGTGGTGGCTTCTCACACTCGGTGCACTCACTGGGATTGGTGGTGGGGTGTTC
TCTGGATATATGCCGTCCACGGGATACTTCTTCCCCAAGGCAAAATCGGGCACTGCGCTG
GGCATTCAGGCAGGTATCGGCAACCTCGGCGTCTCGATAATTCAGTTCATGGGCCGATGG
GTCATGGGTTTCGGTCTGCTGGGCATTGGTTTCCTCACCCCGCAGCGCACCATTGAAGGC
ACCACGGTGTTTGTGCACAATGCTGCGATTGTGTTGGTCCGGTGGACTATTCTCGCGGCC
GTTTTATCGTTCCTGTTTCTTAAAGATGTCCCAGTCACCGCAAATTTCCGGCAACAGATC
GATATCTTTGGCAACAAGAACACATGGATTTTGTGCATTATCTACTTGATGACATTCGGT
GCCTTCGCCGGTTTCGCCGCGCAGTTCGGTCTGATCATCAACAACAACTTCGGCATCGCT
TCCCCGATGGCAGAGACTTATCCAGCTGAGATGCTTCACGCCGGTGCTACGTTCGCGTTT
CTTGGACCTTTGATTGGTGCTTTGGTGCGTGCTGCATGGGGTCCACTGTGTGACAGATTC
GGTGGAGCTATCTGGACGTTTGTCGGTGGCATCGGAATGACTATCGCCACTGCAGCTGCC
GCAATCTTCCTAAGCAGAGCGGAGACACCTGATGATTTCTGGCCATTCCTGTGGTCCATG
CTTGCCCTGTTCTTCTTCACCGGTCTGGGCAATGCTGGCACCTTGAAACAAATGCCCATG
ATTTTGCCTAAACGCCAAGCAGGTGGCGTGATCGGCTGGACCGGTGCCATTGGTGCCTTC
GGCCCCTTCATTGTCGGTGTCTTGCTCTCCTTCACTCCAACTGTCGCGTTCTTCTGGGGC
TGCGTGGTGTTCTTCATCATCGCCACGGCTTTGACCTGGATCTAGTACGCCCGCCCGAAC
GCTCCATTCCCGGGA
>RXA01303-downstream
TAAACCGAAAGGCCAATCCATGA
>RXA01323-upstream
CACGTGGTTTACGCCAGGCATGTTCCCGCGAAGGGTTGACCCATACCCCTAGGGGGTATA
CAGTGAGTCATGTAAACATACTCGCAGAAGGAGGGATCCC
>RXA01323
ATGGCTCAGACACCCGCCAAAATCCCGGCGGCACTGAATTTCATTGACGTCGACCTCGGC
GTTACGGGCATGACCTGCACTTCTTGCTCCGCGGGCGTCGAGCGCAAACTGAACAAGCTC
GACGGCGTTGAAGCAACCGTCAACTACGCGACGGAATCCGCACAGGTCAGCTACGACCGC
TCAAAGGTCAGCCCTGAACAGCTGATTAAGACTGTTGAGGACACCGGCTACGGTGCTTTC
ACGATGGCTTCCGCAGCTGCCGAATCAGAAGAGGACAACGCTCCAGCTGACAGCGGCCAG
TCCCGGATCGACGCAGCTCGCGACCACGAAGCAGCCGACCTGAAACACCGCGTGATCGTC
TCTGCACTGTTGTCAGTTCCTGTGGTTTTGGTCAGCATGATCCCGGCGCTGCAATTCAAC
AACTGGCAGTGGGCCGTACTCACTTTGGTCACCCCGATTTTCTTGTGGGGCGGTTCACCG
TTCCACAAGGCAACGTGGGCAAACGTGAAGCGCGGTTCCTTCACGATGAACACCCTGGTT
TCACTCGGCACGTCCGCTGCTGACCTGTGGTCCCTGTGGGCTTTGTTCATTGAAAATGCT
GGTCACCCTGGCATGAAGATGGAGATGCACCTGCTGCCGTCGGCCTCCACGATGGATGAG
ATTTACCTCGAAACCGTGGGGGTCGTTATTACGTTCGTGCTGCTTGGACGCTGGTTTGAG
ACAAAAGCTAAGGGCGAATCTTCGGAAGCTCTGCGCAAGCTGCTGGACATGGGCGCCAAA
GATGCAGTCGTCTTACGTGACGGCGCCGAAGTCCGGGTTGCTGTGAATCAGCTTAAACTC
GGCGACGTTTTCATCACCCGCCCCGGCGAGAAAATGGCCACCGACGGTGAAGTCGACGAA
GGTTCCTCCGCAGTCGACGAATCCATGCTCACCGGCGAATCCATCCCGGTTGAAGTCACC
AAGGGCTCCAAAGTTACCGGCGCAACGCTGAAGACTTCCGGCCGCCTCATGGTGAAAGTA
ACCCGCATCGGGGCCGACACCACCCTGTCGCAAATGGCTAAACTGGTCACGGACGCAGAG
TCCAAAAAGGCCCCTGTCCAGCGTCTTGTTGACCAAATCTCGCAGGTTTTGGTTCGCGTT
GTCATCGTAATTGCTATTGCGACGCTGATCGGGCACCTGGTGTTCACGGACGCCGGCCTC
GCCCCAGCATTCACCGCAGGAGTCGCCGTGCTCATTATCGCGTGCCCTTGTGCCCTCGGC
CTGGCAACCCCAACCGGACTTCTGGTCGGAACCGGCCGCGGCGCGCAACTTGGTCTGTTG
ATCAAGGGCGCTGAAATCCTCGAATCCACCAAAAAAGTCGACAGGATCGTCCTCGACAAA
ACCGGCACCGTCACCACCGGCACCATGTCCGTCACCGACGTCAGCGCCATCAACTACAGC
GAAACCGAAATCCTGGAATTCGCTGCAGCCGTCGAGTCCGCCTCGGAACACCCCATCGCC
CAGGCAATGGCCAAGGCCGCCGAACACGAGGAAGTCACCGACTTCCAAAAGACGGCAGGT
CAGGAAGTCACCGGTGTAGTCCGCGGACAGGAGGTCCGCGTGGGCAGGCCTTCAAGCACG
CTTATCGACGGCCTGCTCCACCCCTTGCAACACGCCCAAAAAATCGGCGGAACCCCCGTA
GTCGTCACGATTGACGGCGTAGATTCGGGAATAATCACGGTCCGCGACACCGTCAAAGAC
ACCTCCGCCGAAGCAATCCGCGGACTCAAGGAACTGGGACTCACCCCAATCCTACTCACC
GGAGACAATGAAGGCGCAGCTAAATCCGTAGGCGCTGAAGTCGGCATCGACCAAGTCATC
GCCAACGTCCTCCCCCACGAAAAAGTCCAAAACGTAGAAGCCCTCCAAGCACAAGGCAAA
AACGTTGCGATGGTCGGGGACGGCGTCAAGGATGCCGGAGCTCTTGCCCAAGCTGAGGTC
GGAGTCGCCATGGGAGCCGGCACCGACGTAGGCATCGAAGCCTCCGACATCACCCTCATG
AACAACGAGCTCCGATCCGCAGTCGACGCCATCCGACTGTGCCGTAAAACGCTCGGCACC
ATCAAGGGAAACCTTTTCTGGGCTTTCGCCTACAATGTTGCACTAATCCCAGTAGCGGCG
ATCGGACTCCTCAACCCAATGCTTGCCGGCATTGCGATGGCCTTCAGTTCAGTTTTCGTC
GTCTCCAATTCCTTGGGTCTGCGAGGATTCAAAGCAAGGAGCAAC
>RXA01323-downstream
TAATGTCCAACAGCGAATGCCAC
>RXA01338-upstream
ATCCTTGCCTTGCCAAGGGAAGCCTGTACATGCTGGTCAGGGACATTTTTATGGGTGATA
ATGGGGTTTATGAATAAAAACTTATACCCCAAATCCCTGG
>RXA01338
ATGTTATTCATCCGCTCATTTGATGGCATCATCACTGTCGCAGCCCTTGTTGCCATCGCA
ATACATCTCATTTTATGGCTGGCTCTAGATCTAGATGGCCTTGCTAAAAACTGGCCTTTA
ATAGCCATCGTTATCGTAGGTGGCATTCCGTTGATGTGGGATGTGCTGAAATCAGCCATT
AAAACTCGCGGTGGGGCGGATACTTTAGCAGGAGTCTCCATCATTACTTCTGTGTTGTTA
GGGGAGTGGTTGGTTGCCGCGATCATCGTGCTCATGCTCTCTGGTGGTGAAGGGCTAGAA
GAGGCAGCATCACGGCGAGCCAGTGGCACCTTGGACGCACTTGCCCGGCGCGCACCAAGT
ACAGCTCACCGCGTGTTGGGTGCAAGGATTGTTGATGGAACCGAAGAGATGGCGGTGGAA
GAGATCACGGTTGGTGATTTAGTGGCGGTGGTCCCGCATGAACTTTGTCCCGTGGATGGT
GAAATCGTGGGAGGCCACGGCACGATGGATGAGTCTTATCTCACGGGTGAGCCCTATGTG
GTGAGTAAATCTAAAGGTTCGCAAGCAATGTGGGGTGCAGTCAATGGTGATACTCCGCTG
ACGATTGTTGCGACAAAGCTTGCCCATGATTCCAGATAGGCCCAAATTGTTGGTGTACTC
CATGAAGCAGAAAACAACCGCCCAGAAATGCGCAGGATGGCTGACCGTCTTGGCGCGTGG
TATACGGTGATTGCACTTGCCCTCGGTGGTCTTGGCTGGATTGTCTCCGGCGACCCAGTG
AGGTTCTTGGCTGTTGTCGTTGTCGCCACCCCATGTGCATTGCTCATTGCAGTGCCAGTG
GCGATCATCGGTGCGATTTCTCTTGCGGCTCGTCGGGGCATCATCGTGAAGAACCCTGGA
ATGCTGGAAAACGCTTCAGGAGTAAAGACAGTGATGTTCGATAAGACTGGAACGCTCACC
TATGGCAGGCCAGTGATTACTGATATCCACACTGCTCCCGGAGTTGAGGAAGATACAGTC
CTAGCTTTGGCTGCTTCAGTAGAGCGCTACTCCAGACACCCGTTGGCTGACGCGATTCGT
GAGGGCGCAAAAGCCAGGGAACTTCATCTGCCTGATGTAGTGGAAGTATCGGAACGTCCA
GGACAGGGACTAACCGGCACGGTGGGCGAGGACCTGGTTCGAATAACCAATAGGCGCAGC
ACACTAGAAATTGATGCAGACAGCAAGAACTACATTCCGGTGACAAGTTCCGGCATGGAA
TCTGTGGTGCTTGTTGATGATAAATATGCAGGACTCATTCGCCTCGGGGATGAACCTCGT
GCATCTGCCAGTGAGTTCATCGCGCACTTGCCCAAGAAGCACAAAGTGGACAAGCTCATG
ATTATCTCTGGTGATCGCGCATCTGAGGTTCGTTACCTTGCGGACAAGGTTGGCATTGAT
GAGGTACACGCAGAGGGCTCACCGGAAGACAAGCTGAACATTGTTAATCGGCATAATGAG
CACGGGGCCACCATGTTCTTAGGTGATGGAATCAACGATGCGCCAGCGATGGCCGTTGCC
ACCGTTGGTGTCGCGATGGGAGCAGACTCCGATGTCACGTCCGAAGCAGGAGATGCTGTG
ATTTTGGATTCTTCCCTGGAACGTGTCGACGATCTGGTCCACATCAGTGCACGGATGCGT
CGAATAGCGTTGCAATCTGGGGGCGGTGGCATGGCGTTGAGTGTCATAGGAATGATCCTC
GCGGTATTTGGATTCTTGACGCGAGTGATGGGTGCGATCTTCCAAGAGGTCATTGACGTG
CTGGCTATCCTCAATTCGGCTGGGGTCGCACTGCCACGCGGAGCGATTAGTGATTTTGAT
ACGCAAGAAAAAGTTTCT
>RXA01338-downstream
TAGCAGGGTAACCTAAATGTCGT
>RXA01395-upstream
CTCAAAAGCACTGATAAAAGCAGTCAACCCACCTCGGGTTGGCTGCTTTTTTGCATCCAG
ATGCACAAAGCGGTGGCACAAACGAGACAAAGTGAGCACA
>RXA01395
ATGGCTGTCATGGCATATCAACCAGCAGACAATCGCTATGACGACATGATCTACCGGAGG
GTGGGAAATTCTGGGCTGAAGCTTCCGGCAATTTCGCTTGGGCTGTGGCACAACTTCGGT
GATGACAAGCCGGTTTGAACGGAGCGCAGCATTATTCACCGCGCGTTTGATAGGGGAGTC
ACTCACTTCGATTTGGCTAATAACTATGGACCTCCAGCAGGTTGCGCAGAGACCAACTTT
GGCAGGATTTTGCGTGAGGATCTCAAAAGGGACCGCGATGAGTTGATCATTTCTTCCAAG
GCGGGTTGGGATATGTGGCCTGGACCTTATGGTTTTGGTGGTTCCCGAAAGTATCTAGTG
AGTTCCCTTGATCAGTCCCTGACTCGCCTCGGCTTGGATTACGTGGATATTTTCTATCAT
CACCGCCCGGATCCAGATACTCCTTTGGAAGAAACCATGTACGCATTGCGTGACATTGTT
GCGTCTGGAAAGGCTCTTTAGGTGGGTATTTCTTCCTACGGTCCAGAGCTCACAGCGGAG
GCGGCTGAGTTCATGGCGGAGGAGGGCTGCCCGCTTCTGATTCATCAGCGAAGCTATTCC
ATCATTAATCGTTGGGTGGAGGAACCGGGCGATGACGGTGAGAACTTGTTGCAGTCAGCT
GCCAACAATGGTCTTGGCGTCATTGCTTTCTCACCACTTGCGCAGGGCCTGCTCACGGAC
AAATATCTCGATGGAATTCCAGAGGGTTCCCGCGCCAGCCAGGGTAAGTCCCTKTSTKAC
GGSWTGTTGAACGTGAACAATATTGATWTGGTCCCMARSYTNAWKRSAWTTTCCMARRAM
ACCGGGCAGTCCTTTNNCCNAAAGGNCTTTTGTTGGGTTGTTGCCCAACCAAGGAAAGTA
CGGCGCCGGATTACCGTGACCAGTGCATTGATTGGTGCTTCGTCAGTTGAGCAGCTGGAC
AACAGCCTTGATTCACTCAACAACTTGGAGTTTTCTGACGCCGAGTTGGAGGCGATCGAT
GAGATTTCCCACGACGCCGGCATCAACATTTGGGCGAAGGCCACCGATTCCAAAACCCGC
GAAAAC
>RXA01395-downstream
TAACCCATCAACATCAGTTTGAT
>RXA01411
TTCATTGCGCAGGTAATGCTTGGAATGGGGGCGGTTACCGCTAACTGCGTTACCTCAGTA
ATGATGGCCGAGGTCTTCCAAGAGGTCACGCGCGGTACTTCCGGCGGCATTACCTACAAC
GTCACTTACGCAATCTTCGGCGGCTCGGCTCCATTTATCTCCACCGCATTGGTCTCCTGG
ACCGGGAGCCCGCTGGCCCCTGCGGTATAGATGATCATCATTGGGCTCTTCGCCTTCACC
GCGTCGCGCTTCATTCCTGAAACCTCCCCAGTTTTTGTCAGCGCAACCCCGGCCATTAAG
GCACCAAAGGTGCTGGTCAACGCGGGT
>RXA01411-downstream
TAAACCACGGTTTTCGACGAAAA
>RXA01454-upstream
GGCCAGGACTTTGGCGTTTCGGATCAGCAGTTCGGCTTGGATTATGGATTCTACGCGTTT
GATCTTCCGATGCTTCGCCTCATCGCTTGACTCACTGTCG
>RXA01454
ATGATGTTGATCGTTGCTTTCGTGATCGCAGTGGTTGGCCATTACCTCATGGGTGGCATT
GGCGCTGGAAACCAGATGACGGGCCAGAAGTCCTTTGTATCCCGTGGTGCGCGCACTCAG
CTTGCGGTAACTGCTGGTCTGTGGATGCTTGTTAAGGTCGCTGGCTACTGGCTGGATCGC
TATGACCTGCTGACTAAGGAAAACTCAACCTTCACAGGTGCAAGCTACACCGACATCAAT
GCACAGCTGCCAGCGAAGATCATCCTG
>RXA01455-upstream
AGAGTGGGGGAGAAACGCATAATCACGTACATATAAGGATATTGTGTTGTCGACTGGTCT
CACACCTCGTCCCCAACCGATCAAGCGACCTCCCAAGGGG
>RXA01455
GTGACATGGATCTTCGCGATTATCGCGTTGGTCATTCTCATCGGCGCAATGAGTGTTGGC
TTCTATACGGACTGGCTTTGGTTCGGTGAAGTCGATTTCCGAGGCGTTTTCAGCAAGGTT
ATTGTCACTCGCATTGTTCTCTTTGTGATCTTTGCGCTAATTGCTGGGTTTGTCACATGG
CTTGCTGGTTATTTTGTGACAAAACTTCGACCTGATGAGATGTCGGCGTTTGATACCCAG
TCGCCTGTGTATCAGTACCGTGAGATGATCGAAAACAGCCTTCGTGGCGTTATGGTGATC
ATTCCAATTTTCGTCGCGTTGCTGGCTGGCCTAATTGGTCAGCGTTCGTGGCGCACCGTT
CAAATGTGGCTGAATGGCCAGGACTTTGGCGTTTCGGATCAGCAGTTCGGCTTGGATTAT
GGATTCTACGCGTTTGATCTTCCGATGCTTCGCCTCATCGCT
>RXA01455-downstream
TGACTCACTGTCGATGATGTTGA
>RXA01625-upstream
GGGAGCGAAGTTCCCTGGGTTAAATTAACCACTTGCAGTATACCCTAGTGGGGTATATTG
TCTGCTGTTAGAAGATACCCGACAGAAAGGGGCCAATAAT
>RXA01625
ATGGCTATCAAGAACTACAGCGTCGAAGGCATGACTTGTGGACACTGCGTCTCCTCCGTA
AAGGAAGAGGTCGGAGAGGTTGCTGGCGTCACCGCTGTGGACGTCACCCTAGAAACCGGT
GCCGTGCAGGTTACCGGCGAAGACTTCACCGACGAGGCTGTCAAGGCTGCTGTCGTTGAG
GCTGGCTACAAGGTTGTTGCA
>RXA01625-downstream
TAAACCCCTGAAAAGTTTAAAGC
>RXA01756-upstream
GCTTCAAACAGGATTTAATCTAAAATCTTAAACCTCGTATTTTCCCTGATAGGCTCAGAT
GCGCCTGAAATCGGGCTTGTTGAGGGGAGAGGTGTGTGAC
>RXA01756
ATGAAAGAGTTGGAACTGGGCGAGGCGAGGGACGTCGCTGCAACGTTGGAAGCGATGCCG
ATCCAGGAGGTTATTGATCAGGTTGAGCGAACTTCTATAACTAAAGGTGCGGTACTGCTG
CGTCTGCTGAGTAAAGATCGATGGTTGTTGGTCTTCGATGCTCTTGGTCCGCGACTCCAG
GCTGATCTCATTGGTGCTTTTCAGGATGCGGAAGTGCTGGATTATTTCGCTGACCTTGAC
CCTGATGACCGCGTTTGACTGCTTGATGAGCTGCCGGCGTGGATCGCTGACGAGTTGCTT
CGCAGTCTCGATCCGCAGGAAAAGCAGGTCACGGAGCTGGTCTTGGGTTACGCAAAGGGG
TCGGTTGGACGTTGGATGTCGCCCCAGGTTTTATTGCTTTTCGACGACATGTCCGTCGCC
GAAGTCTTAGATTTTGTGCGCAATCATGCTGCTGAGGCTGAGACGATTTATGCCTTACCT
ATTGTGAACCGTGCTCGCCAAGTGATGGGCGTGGTGTCGTTGCGAAAGCTGTTCATCGCA
GATCCCACTCTAAAAGTCTCGGAAATCATGGTGCGTCCTGTTTCGGTGTTGGCGTCCGCG
GATATTGAAGAAACGGCCCGCTGGTTCCTACAGTTGGACCTCGTTGCGATGCCCGTTGTG
GATGAATCGAACATGCTCTTAGGAGTGCTGACCTTCGATGATGCGCAAGACATCGTGGAG
CAAGCCGACTCTGAGGAGTCCGCTCGCAGTGGTGGTTCGGAACCTGTCCAGCAGCCGTAT
CTATCCACGCCGATTCGGAAACTGGTGAAGTCCCGCATCGTATGGCTTCTGGTTTTGGCA
GTGTCAGCAATTTTGACGGTTCAAGTTCTTGATATTTTCGAAGCCACCTTGGTTGAAGCC
GTGGTACTGGCATTGTTCATTCCTTTGCTCACTGGTACTGGCGGAAACACCGGAAACCAA
GCTGCAACAACCGTGACCCGTGCGCTCGCATTGGGTGACGTCCGAAAATCAGATGTCTTC
CGCGTCTTGGGCAGAGAAATCCGAGTCGGCCTCATGCTCGGGGCATTGTTGGGTGCCGTT
GGATTTGTGATCGCATCGCTTGTTTACGGCATGCCCGTAGGCACTGTCATCGGTCTGACA
TTGTTGGGGGTGTGCACGATGGCCGCATCAGTTGGCGGAGTAATGCCAATTATTGCCAAG
GCGATCGGAGCGGACCCAGCGGTGTTCTCTAATCCTTTTATTTCAACCTTCTGTGATGCA
ACAGGTTTGATCATCTACTTTGCAATTGCCAAGTTGGTGCTCGGAATC
>RXA01756-downstream
TAAAAGATTTTTGCTTTTCGACG
>RXA01808
ATGCGCGGGGGTGCACCAGCGCGAACCTCAAAGCCTGGATTCCGGCTTGAAGCCGCGGAA
GCTTTGATCGCAGAAGTGCCAGCGCCACGCGACAAAGTCGAGCTGATGGCATTTTCCAAG
TCCAGGGAAGGCCGCGTTGTCATTGAACTTGAAGACGCCACAGTAGCCACCCCTGATGAT
CGCATCCTGGTAGAAGACCTCACCTGGCGTTTGGCTCCAGGAGAGCGCATCGGTCTTGTC
GGCGTCAACGGCTCCGGCAAAACCAGGCTGCTGCGGACCCTTGCGGGCGAGCAGCCACTT
CAGGCAGGCAAACGCATCGAAGGCCAAACCGTCAAACTGGGATGGCTCGGCCAGGAACTC
GATGACCTAGAGCTCAGCCGCCGACTCATCGACTGCGTTGAAGATGTCGCTTCCTACGTG
ATGATGGGGGACAAGCAGGTCTCCGGTTCCCAATTGGGAGAACGGGTCGGATTCTCACCC
AAGAGGCAACGCACCCCAGTTGGTGACCTGTCCGGTGGTGAACGCCGCCGACTCCAACTC
ACCCGCGTGCTCATGGCCGAACCAAACGTGCTGCTCCTCGACGAGCCCACCAACGACCTG
GACATTGACACCCTCCAAGAGCTGGAATCCCTTGTCGACGGATGGCCAGGCACCATGGTG
GTTATCTCCCACGACCGTTACCTCATCGAACGCGTCACCGAGTCCACCTGGGCACTCTTC
GGCGATGGCAAGCTCACCAAGCTGCCAGGCGGAATTGAAGAGTACCTGCAGCGACGAGCA
GCGATGGCCGCGGCCGAAGACAGTGGAGTGCTGAACTTGGGTGCGGCCACGCAGGCTGGA
ACCTTTTCTGCTGCAACAGAGCAGGCTGCCACTTCTGTGGAAAGTTCCGGAATTTCTTCC
CAAGAACGCCACCGCATCACCAAGGAAATGAACGCCCTGGAGCGCAAAATGGGCAAGCTT
GACCAGCAAATGGACAAGCTTAATCAGCAGCTCGCTGATGCAGCGGAGGCCATGGACACC
ATAAAGCTCACCGAGCTGGACACCAAGCTCCGCGCAGTGCAGGAAGAACACGGCGAGCTG
GAAATGCAGTGGCTGGAACTCGGCGAGGAAATCGAGGGC
>RXA01808-downstream
TAGTTCATGCCGTCGGCAGGCGA
>RXA01822
ATGGCCAGACAAAATAGCAATACCGGCGGGTTGCGTCTGGTGTTGGTTGGTATCGGAAGA
GGTGCATTTTTGGGTGCTGCTCGTGATTTCTTCATGGTGCGCGCAGATATTACGGGTGCT
TCGACGGTACAGCTGTGGTCTGCCGGTTCGTTGAGCGGGCGCGACTGGAATCATGCCCTG
TTGGTGTTGATTTCGTGTGCAGTGATTGTGCGAGCACTGTGCATTATTGTCCGCCGTTTA
CGCCTGATGGAAATGGGTGATGATGCAGCTGGGGCACTTGGAATTTCAGTGGAGAGAACA
CGGTTGATAGCCATTTTGTTGGCTGTGCTGCTGGTGGGGATCGCCACCGCAGCTGCAGGT
CCCATCGCTTTTATTGCACTGGCAGCACCTCAGATTGCCCGGGCTCTGGCCCGGGAGGAT
GGAGTGCTGGTGGCTGCGTCGATAAGCATTGGCTCTGGGCTGTTAGTTGCGGCGGATTGC
CTAGAGCAACACGTTGATACTGAGCTGCACACGCCCGTTGGCCTGGTGACCAGTTTGCTG
GGCGGGGTGTATTTGATGTGGCTTTTGAGCCGAAAGGAGGCA
>RXA01822-downstream
TAAATGCTGCAAGCGCATGATCT
>RXA01890-upstream
GCTGAGGTTGAGACCAAGCTGAACACCATCTACACCCGCGACATCGAACCACTTATTTAA
TCCGAGCACTTCAGCTACAGCTATTTAAGGAGGCTGTGAC
>RXA01890
ATGGCGTCAATCGTCTTTGAAAACGTCACACGCAAATACTCTCCGGGCGCACGCCCGGCC
GTCGAGAAGCTTAATTTGGAAATCGCGGACGGCGAGTTGCTAGTTCTCGTTGGACCCTCA
GGCTGTGGAAAGTCCACTTCTTTGCGCATGCTGGCTGGTCTTGAGCCTATCGACGAGGGA
CGTCTACTCATTGATGGTAAAGACGGCACGGAACTGCGTCCGCAGGATCGTGACATCGCT
ATGGTCTTCCAGAGCTACGCGCTGTACCCGAATATGACTGTTCGGGACAACATGGGCTTT
GCGCTGAAGAATCAGAAGGTGGCTAAGGCTGAGATCGAAAAGCGTGTTGCTGAAGCCTCA
CGCATTCTGCAGCTGGATCCGTATCTTGATCGTAAGCCTGCAGCTTTGTCTGGTGGTCAG
CGCCAGCGCGTGGCCATGGGCCGTGCAATTGTGCGTGAGCCATCGGTGTTCTGCATGGAT
GAGCCACTGTCCAACCTAGATGCGAAGCTGCGTGTGTCTACGCGTGCGGAGATCTCTGGT
TTGCAGCGTCGCATGGGCGTGACCACGGTGTATGTGACTCACGATCAGGTCGAGGCCATG
ACCATGGGTGATCGCGTCGCTGTGCTTTTGCTCGGTGTGCTGCAGCAAGTAGACACCCCG
CAGAACCTGTACGACTACCCAGCAAATGCGTTCGTCGCCAGCTTCATTGGTTCCCTTCCA
>RXA01890-downstream
TGAACTTGATTGAGGGCACCATC
>RXA01900-upstream
AAAGGTGACACGCCTTAGATTCTTGTGGTCTGACCATGAGGTTGGGCCAATCGGTTTCAG
CCCGTTTACTCGCGCCGTCCGTTTCAGAGAAGAGGTCACC
>RXA01900
ATGACAACCGCAGTAGATCAAAACTCACCGCGCAAGCAGCAACTCAACAAGCGCGTCCTG
CTGGGCAGCTTGAGTGGCAGCGTTATGGAATGGTTGGACTTCCTGGTTTACGGAACCGTC
GCCGCGCTGGTCTTCAACAAGATGTACTTCCCCAGCGGCAACGAGTTCCTCTCCACAATC
CTGGCGTACGCATCCTTCTCCCTGACCTTCTTGTTCCGCGCCATTGGTGGCGTCATCTTC
GCCCACATCGGCGACCGCATTGGGCGTAAGAAGACCCTGTTCATCACCTTGATGCTCATG
GGTGGCGGCACCGTGGCGATTGGTTTGCTGCCCGACTACAACGCCATCGGCATTTGGGCA
GCAATCCTTCTGATGTTCCTCCGCATTTTGCAGGGCATCGGAATTGGCGGCGAATGGGGT
GGCGCACTGCTCCTGGCATACGAATACGCTCCAAAGAAGCAGCGTGGGCTCTACGGCGCA
GTTCCTCAAATGGGCATTTCCCTGGGCATGCTGCTTGCAGCTGGCGTGATCTCTCTGCTC
ACCCTCATGCGGGAAGATCAGTTCCTCACCTGGGGCTGGCGCATCCGATTCGTCGGATCC
ATCCTCCTAGTGTTCATCGGCCTGTTCATCCGAAACGGCCTTGATGAAACCCCCGAGTTC
AAGCGTATCCGCGATTCCGGCCAGCAGGTAAAGATGCGTCTGAAGGAAGTTCTGACCAAG
TACTGGCCAGCCGTTCTGGTCTCCATCGGCGCAAAAGCTGCCGAGACGGGCCCCTTCTAC
ATCTTCGGCACCTACATCGTTGCTTACGCAACCAACTTGCTGAACATCCGCGACAACATT
GTCCTTCTGGCAGTTGCTTGCGCCGCCCTCGTTGCCACCATCTGGATGCCACTGTTCGGA
TCCTTCTCCGAGCGCGTCAACGGTGCAGTGGTGTACAGGATCTGTGCATCCGCAACCATC
GTGCTGATTGTCCCTTACTACTTGGTCCTCAACACCGGCGAAATTTGGGCACTGTTTATC
ACTACCGTGATTGGCTTCGGCATCCTCTGGGGTAGCGTCAACGCAATCCTCGGAACCGTC
ATCGGAGAAAACTTCGCACCTGAGGTCCGCTACACGGGCGCTACCCTGGGTTAGCAAGTC
GGAGCAGCACTCTTCGGCGGTACCGCACCCATTATCGCAGCATGGCTGTTCGAAATCTCC
GGCGGACAATGGTGGCCAATCGCCGTCTACGTCGCTGCATGTTGCCTTGTCTCTGTGATC
GCCTCGTTCTTCATCCAACGCGTCGCGCACCAAGAGAAC
>RXA01900-downstream
TAAAATCTAAGTAAAACCCCTCC
>RXA01939
TCTACAAGCGGCACCGATCTTACGTCCTTGAGCCACAAGGAAATCTTCCAAATGCGACGC
AAACTGCAGGTGGTGTTCCAGAACCCCTACGGCTCGCTTGATCCGATGTACTCCATCTAC
CGGTGTATTGAGGAACCGCTGACCATCCACAAGGTTGGTGGAGACCGCAAGGCACGCGAA
GCTCGCGTCGTTGAACTTCTCGATATGGTGTCCATGCCCAGGTCCACCATGCGCCGCTAC
CCCAACCAGCTTTCCGGTGGCCAACGTCAGCGCATCGCCATCGCCCGTGCATTGGCACTG
AATCCAGAAGTGATCGTGTTGGATGAAGCGGTTTCCGCTTTGGACGTGTTGGTTCAGAAC
CAGATCCTCACCCTGCTTGCAGAACTTCAGCAGGAACTGAAGCTCACCTATTTGTTCATC
ACCCACGACTTGGCCGTTGTTCGACAAACCGCCGACGATGTTGTGGTGATGCAAAAGGGA
CGAATCGTTGAAAAGGGTCGTACCGACGACATCTTCAACGATCCTCAGCAGCACTACACC
CGCGATTTGATCAATGCGGTACCTGGTCTGGGAATCGAGTTGGGTACTGGAGAAAACCTG
GTT
>RXA01939-downstream
TAACCCGCACAGCCTCACTAAAC
>RXA01972-upstream
ACGCGTTGCTGGATATCACCCTGGCCGTCGATGACAACGCCGAATGCATCGACGCCGGAT
GCGCCGTACCTGGGTGTGTCACTGGACAGGAGAGTGCGTA
>RXA01972
GTGGCAACCGGTCTACTGTCGGCGATTGGTCTGTTTATCGCCACCAATATCGACGACATC
ATCGTGCTCTCGCTGTTTTTTGCCCGCGGGGCGGGGCAAAAAGGGACCACGCTTCGGATT
CTGGCTGGTCAGTACCTCGGCTTCATGGGCATCCTCGCGGCCGCAGTCCTGGTCACGGTG
GGGGCAGGAGCATTCCTACCTGCTGAGGCGATCCCGTACTTCGGACTAATTCCCCTGGCC
CTGGGACTATGGGCGGCCTGGCAGGCCTGGCGAAGCGATGATGACGACGATGATGATGCG
GAGATCGCCGGGAAAAAGGTGGGTGTGCTGACCGTCGCCGGTGTGACGTTTGCCAACGGT
GGCGACAATATCGGCGTCTACGTCCCGGTCTTCCTCAACGTGGACACTGCCGCCGTCATC
ATCTACTGCATCGTTTTCCTCGTCCTGGTGGCAGGCCTGGTCCTGCTGGCAAAGTTCGTG
GCCACCCGCCCGCCCATCGCAGAAGTCCTTGAGCGCTGGGAGCACGTGCTGTTCCCGATC
GTCCTGATCGGCCTGGGCATCTTCATCCTCGTCAGCGGCGGCGCGTTCGGCCTC
>RXA01972-downstream
TAATAAGCCCATCCCGAGGGCCC
>RXA01986-upstream
GCCACGATTAAAGACATTGGTGATGTGAATCACTGGGTACTACATCGTGTTTCGTGACGC
TGCACCTCCAAGTAAGGGCACGACAAACTTAGGAGAGAAG
>RXA01986
ATGGCTAGTACCTTCATTCAGGCCGACAGCCCTGAAAAAAGTAAGAAGCTGCCCCCACTC
ACAGAAGGTCCGTATAGAAAGCGGCTATTCTACGTTGCACTAGTTGCGACGTTTGGTGGG
GTGCTCTTCGGATATGACACCGGAGTAATCAACGGTGCACTCAACCGAATGACACGTGAG
CTCGGACTAACCGCGTTCACCGAGGGTGTTGTAACTTCTTCCCTGCTGTTTGGTGCAGCA
GCTGGTGCGATGTTTTTCGGTGGCATTTCCGACAACTGGGGTCGCCGGAAAACAATCATC
TCACTTGCAGTAGCTTTCTTTGTCGGCACCATGATGTGCGTGTTTGCTCCATCTTTTGGA
GTAATGGTTGTCGGACGTGTGCTTCTTGGACTCGCAGTTGGTGGCGCTTCCACTGTTGTC
CCTGTCTACCTGGCTGAACTTGCTCCTTTTGAAATCCGTGGCTCAGTGGCTGGCCGTAAT
GAGTTGATGATTGTTGTTGGTCAGCTCGCAGCTTTTGTCATCAATGCGATTATTGGAAAT
GTTTTTGGACACCACGATGGTGTGTGGCGCTAGATGCTGGCAATTGGCGCAATCCCAGCA
ATTGCCCTCTTCTTTGGA
>RXA01995-upstream
CCGACGGAAAGGCATGCGCCTGCGTGTCTCGAGTAGTCTCCTCCCCTTCCTCGTCCCCAA
CCTCGACCATTACGGTCGCCGTCTCCTAAAGGAGGCTGGC
>RXA01995
ATGGATATCCGCCAAACAATTAACGACACAGCAATGTCGAGATATCAGTGGTTCATTGTA
TTTATCGCAGTGCTGCTCAACGCACTGGACGGCTTTGATGTCCTCGCCATGTCTTTTACT
GCGAATGCAGTGACCGAAGAATTTGGACTGAGTGGCAGCCAGCTTGGTGTGCTGCTGAGT
TCCGCGCTGTTCGGCATGACCGGTGGATCTTTGCTGTTCGGTCCGATCGGTGACCGTTTC
GGCCGTAAGAATGCCCTGATGATCGCGCTGCTGTTCAACGTGGTGGGATTGGTATTGTCC
GCCACCGCGCAGTCCGCAGGGCAGTTGGGCGTGTGGCGTTTGATCACTGGTATCGGCATC
GGCGGAATCCTCGCCTGCATCACAGTGGTGATCAGTGAGTTCTCCAACAACAAAAACCGC
GGCATGGCCATGTCCATCTACGCTGCTGGTTACGGCATCGGCGCGTCCTTGGGCGGTTTC
GGCGCAGCGCAGCTCATCCCAACATTTGGATGGCGCTCCGTGTTCGCAGCCGGTGCGATC
GCAACTGGTATCGCCACCATCGCTACTTTCTTCTTCCTGCCAGAATCCGTTGATTGGCTG
AGCACTGGCCGCCCTGCGGGCGCTCGCGACAAGATCAATTACATTGCGCGCCGC
>RXA02033-upstream
TGATCTGCTGTATCAGGTGGTTGATCCAAGAGTCGGTGCTGTTGGGGTTGCTAGCACTAA
GGTTCCAGGGAGCGTGGCTTAAGTGACAACGATCAAAAAC
>RXA02033
ATGCCCCTTTCAGGGAAAATCGGCGGCTTCATCGTTGGCGTTGTATTTGTTCTTGCTGCG
CTGTCTTTCATTTGGACTCCGTTTGATCCAGTTCAAGCTTTCCCACAGGAGCGCCTTGAG
GGAAGTTCTTTGAGGCACCTGTTGGGAACGGATCGTTATGGTCGCGATGTTTTATCCCAG
ATCATGGTTGGTTCCCGCGTCACGTTGTTGGTGGGCATGATTGCGGTGGCGATCGCAGCA
TTAATCGGCACGCCACTGGGTATTGCTGCGGGAATGCGCCGTGGCATGGTGGAAACCTTT
GTCATGCGTGGTGCCGATTTAATGTTGGCGTTCCCAGCACTGTTGTTGGCGATTATTTCC
GGCGCCGTTTTCGGCGCCTCCACGTGGTCCGCGATGGTCGCGATCGGCATCGCAGGCATC
CCTAGTTTTGCCCGCGTGGCTCGTGCAGGCACATTGCAGGTGACCAGTCAGGATTTCATC
GCAGCTGCTCGGCTATCAAAAGTAAGTTCCGCCCGGATCGCGCTTCGCCATATTTTGCCC
AACATCACCAGCATGTTGATCGTTCAGGCATCAGTAGCTTTTGCCCTGGCGATCCTGGCG
GAAGCCGCATTGAGTTTCCTCGGTTTGGGCACGACTCCCCCGGATCCCAGCTGGGGTCGC
ATGTTGCAAACCGCTCAAGCATCCATCGGCGTCACCCCCATGTTGGCGGTGTGGCCCGGT
GCTGCGATCGCTTTGACGGTCCTTGGTTTTAATCTTTTCGGTGATGGTTTACGCGATGCC
ATCGATCCA
>RXA02033-downstream
TAGTATTATAT
>RXA02034-upstream
TCGTGGTGATGTCACCAGAGATCACCGGCATTGATCCCAACGTGGTGTCCGGGGCGTTGG
AATTGTCGTTGATTGGTCGGAAAGAATCCGGGGTAGCGCA
>RXA02034
GTGAGTAAAACAATCGCTTGGACTGTGCTGCGGTACACCCTGACTTTTGTGATGGCCAGC
ATCATCATTTTTGTGCTGATTCGAGTCATCCGCGGTGACCCCGCCGCTGTTGCCCTGGGA
ATTACCGCGACACCAGAAGGAATCGCTGCGTTGCAATCACAATTAGGTACTGATCAACGG
CTTTTCCAACAGTACTTTTCCTGGATAGGTGGAATGCTCACTGGCGATTTCGGCACCTCG
CTGAGCTCTGGCCAAGACCTTTCCCCGATCATTTTTGACCGCTTACAAGTGAGCCTCATT
TTGGTGGGATGCTCCATTGTGTTGTCGTTGCTCATTGCCATTGCACTTGGTGTGCTTTGG
GCCCGGCGCGGTGGCGTGATCATTTCCGGCATCAGCCAGATTGGCATTGCGATCCCTAGT
TTGCTCGCCGGTATTTTGTTGGTCGCTGTCTTCGCGGTTGGTTTGGGGTGGCTGCCCGGC
AATGGCTGGATTCCGCCGTCGGAAAACTTTGGAGGTTTCTTAGCCAGGCTGATCCTCCCG
GTTCTGGCTCTTACTGCTGTTCAAGCAGCAATTTTGACCCGCTATGTCCGCTCTGCAGTA
ATGGATGTAATGGGGCAAGATTTCATGCGCAGCGCGAGGTCGAAAGGTATGAGCTTCAAC
CGCGCGTTGATCATCCACGGTCTTCGGAATGCTGCTCTTCCTGTCCTTACCGTCACTGGT
TTGCAGCTAACAACCTTGGTTATCGGCGCCGTGGTGATTGAACAAGTCTTTGTCATCCCC
GGAATCGGTTCGATGCTGCTGGAGTCCGTGTCCAACCGTGATCTCATCGCTGTGCAATCT
ATTGTCATGCTGCTGGTGGCGTTCACGTTGCTGGTTAATTTGGTGGTTGATCTGCTGTAT
CAGGTGGTTGATCCAAGAGTCGGTGCTGTTGGGGTTCCTAGCACTAAGGTTCCAGGGAGC
GTGGCT
>RXA02034-downstream
TAAGTGACAACGATCAAAAACAT
>RXA02035-upstream
GGATTTTCCATTGGCGGAGGTTCATGCGGCGGGTATCCATTGCCTTCCATTTTAGTTTTC
CATTTACTTTCCCGCATCACACCGACTAATCTCAGAAGCC
>RXA02035
ATGAAGATCACGCGCGGACTCCTGCCATCATTGCTGTTGGCAAGCACAATCGTGGTGTCG
TCATGCTCTGCTGGATCGACTGCGTATCAGCAGCCCCCTGCTGTTGATCAATCATCCATT
GTCATTGCTACCACGGCTGCTGCGGCGTCACTTGATTTCACCAATGCTGCGGGCGCTGCT
ATTCCGCAGGCGATGATGTCCAATATTTACGAGGGGCTTGTGCGCATCGATGCGGAGGGT
GAGATTCAGCCGCTGCTTGCCACGTCGTGGGATATTTCACACGATCGCACCCAGTACATT
TTCCATTTGCGGGAGGGTGTGCTGTTTTCCAACGGCGATCCCTTCAATGCTGATTCTGCG
AAGTTTTCCATTGATCGGGTAAAAACTGACTGGACCAATGGTTTGAAAAGTGGCATGGAT
GTGGTGGAGTCCACCGAGGTGATTGACGATCACACGCTGAAAGTTTCGCTGGTCAGGCCG
TCCAAGCAATGGTTGTGGAGCATGGGTACCGCGATGGGTGCCATGATGACGGAGGGGGGC
GTCGATAAGCTGGCAACTGATCCCGTTGGCACCGGCCCGTACACGGTGACGCACTGGGCG
CCGGGGCGCGCAATTGGGTTCGGCGCGCGGGCCGATTATTGGGGGCAGAAGCCGCTTAAC
GACGCCGCAACCATCCGCTACTTCAGCGATGCGACGGCCTCGACCAATGCGCTGCAAAGC
GGTGACGTGGACGTGATTTCGGCGATGCAAGCGCCCGAACAGCTGGCTACGCTCCAGCAA
TACACCGTGGAAGTGGGCACAACCAATGGTGAGATGTTGGTGTCGATGAATAATCAGCGT
GCACCTTTTGATGATGTGCGTGTGCGCCAGGCGGTGATGTTTGCGATTGATCGCCAAGCC
GTCATTGATACCGCGTTGGAAGGTTACGGCACCGACACTGGTGGCGTGCCTGTTCCGCCG
ACTGATCCGTGGTACGAGAAATCCACGATGTACCCCTACGATCCGGACCGCGCACGGGCA
TTGTTAGAGGACGCCGGCGCCGAGGGAACGCGGATCACCATGTCCATTCCTTCGTTGCCG
TACGCTCAGGCAGCCTCTGAAATCCTGTACTCGCAACTGCGAGATGTTGGTTTTGATCCT
GTGATTGAATCAACCGAGTTCCCAGCCGTCTGGTTGGCACAGGTCATGGGGCAAAAAGAC
TACGACATGTCACTAATCGCGCATGTGGAACCCCGCGACATCCCCACGCTGTTTAGCCCC
AACTACTATTTGGGCTTTGACGACACCGAAACCCAAGCCCTCCTCGCAGAGGCAGACAGT
TCAGCAAACGAAGTGGAATTGATGCAACAAGCTGTCGATCGAATCATGGAACAAGCCGTC
GCCGACAACCTCATGAACGTGGCCAACATCGTGGTGATCTCACCAGAGATCACCGGCATT
GATCCGAACGTGGTGTCCGGGGCGTTGGAATTGTCGTTGATTGGTCGGAAAGAATCCGGG
GTAGCGCAG
>RXA02035-downstream
TGAGTAAAACAATCGCTTGGACT
>RXA02062-upstream
TTGTCTAAACATCGTTTTGGGGTCCGAATGATAGCCCCTTTTAATGCCCCCATTTCGGTA
TCGCTGCGCAACTGTTTTTAGATGGCTAATCTTTGAAATT
>RXA02062
ATGAGAGTCGGAATGATGACAAGAGAGTATCCACCAGAGGTTTACGGCGGCGCTGGCGTG
CACGTCAGCGAATTGACCCGATTCATGCGTGAGATCGCTGAAGTTGATGTTCACTGCATG
GGTGCACCTCGCGATATGGAGGGAGTTTTCGTCCACGGCGTCGATCCTGCCTTGGAAAGC
GCGAACCGTGCGATTAAGACACTGTCCACCGGTTTACGCATGGCAGAAGCTGCAAACAAC
GTGGATGTCGTGCACTCACACACTTGGTATGCAGGTCTTGGCGGCCACCTTGCAGCTCGT
CTCCACGGCATTCCTCACGTGGCTACCGCGCACTCTTTGGAGCCAGATCGCCCATGGAAG
CGTGAGCAGCTTGGCGGTGGATACGACGTGTCCTCCTGGTCTGAAAAAAATGCCATGGAA
TACGCTGACGCGGTCATCGCTGTGTCGGCTCGCATGAAAGATTCCATCCTCGCTGCGTAC
CCTCGCATCGAGCCGGACAACGTGCGTGTTGTCCTCAACGGCATCGACACTGAGTTGTGG
CACCCTCGCCCGACTTTCGATGACGCGGAAGATTCCGTACTCCGCTCCCTAGGCGTTGAC
CCACAGCGGCCCATCGTCGCATTTGTCGGCCGCATCACCCGCCAAAAAGGCGTCGAGCAC
CTCATCAAGGCAGCAGCGCTTTTCGACGAGTCCGTGCAGCTTGTGCTCTGTGCCGGCGCG
CCAGACACCCCCGAAATCGCAGCTCGCACCACCGCCCTGGTGGAAGAACTCCAGGCAAAG
CGCGAAGGCATTTTGTGGGTTCAGGACATGCTGGGCAAGGACAAAATCCAAGAGATTCTC
ACCGCTGCTGACACCTTCGTGTGCCCATCCATTTACGAGCCACTGGGCATCGTGAACTTG
GAAGCAATGGCCTGCAACACCGCAGTTGTCGCATCCGACGTTGGAGGCATCCCTGAGGTT
GTTGTCGAGGGCACCACCGGCGCCCTCGTTCACTACGACGAAAATGATGTCGAAACCTTC
GAGCGCGATATCGCCGAAGCGGTGAATAAAATGGTCGCTGATCGAGAGACCGCAGCCAAA
TTTGGTCTCGCAGGGCGCGAACGTGCTATGAATGATTTCTGCTGGGCAACGATTGCTCAG
CAGACCATTGATGTGTACAAATCCTTGATG
>RXA02062-downstream
TAAAACCGAAAGCCGGGGAACCT
>RXA02068
ATTTTTGTCCCCATGTTGCGTATCGCTGCCATTGAACCGAAAGACATTACTTTGGTTACC
GGTTCTGTATCACTTCGAACCTTTCGCGTGCGCACCGGTGAATTGCAGGTCATGGGCGAT
ATTGTGGGTGCAAAAGTACATACCGATGATCCAGAGCTGCAACAATTCCACGGTCGCGCG
GTAGAAATCGCCGATGTGGAGCTGGAGTTATCGCGCACTCGCGATTGGATCATCACGCGC
GTGGCGGTGCTGGGTGAGCGCGCTAAATTTGGCCGGCGCCCAGTGCTGCACACAGTGCCG
TGGAGTCATATCGACGGCATCACCGCAGGTGGTGTCGGCGAGTCCAATCACACCGCCGAA
CTCATCGCAGGGTTTGAGGATATGAGGCCTGCGGAGGTGGCAAAGCAGCTTTATCAGCTG
CCTACGGCTCAGCGTACCGAAGTGACGGAAGAGCTTGACGACGAAAAGCTGGCGGATATC
CTGCAGGAATTGTCCGAGGACCGCCAAGCCGAGTTGATTGAAGAATTAGACATCGAACGT
GGCGCGGACATTCTGGAGGAAATGGATCCAGATGATGCTGCAGACTTGTTGGGTGAGCTG
CCTGATGACAAAGCTGATGTGTTGTTGGATCTGATGGACCCTGAGGAATCTGCGCCGGTG
GGTCGTTTGATGGATTTCTCCGCGGACACCGTTGGTGCGGTGATGACTCCTGAGCGATTA
ATTATGGATCCTTCCACCACAGTCGCTGAAGCGTTGGCGATGGCCAGAAACCCCGACCTT
GCTACTTCTTTGGCATCGTTGATCTTTGTGGTGCGGCCACCGACGGCCACGCCTACTGGA
AAATACCTCGGCTGCGTGCATCTGCAGAAACTGCTTCGGGAGCCTCCATCAAGTTTGATT
GGTGGCATCGTCGACCCCGATCTGCCACCGCTCTACGCTGATGATTCTCAAGAAACCGCA
GCTCGATTCTTTGCCACCTACAACTTGGTGTGCGGCCCGGTCTTGGATGAAAACCGCCAT
CTGCTTGGTGCCGTAGCTGTCGATGACTTGCTCGACCACATGCTGCCAGAAGACTGGCGC
GACGCCGGAATCCGACCAGGAAAGGAGCACACCCATGGC
>RXA02068-downstream
TGATTTCAACCGCTCTGAATTAG
>RXA02079-upstream
CGGGGAGCCGTGCGGACGCTGCGGAACATTAATCATCGGGGAGAGTTTCATGAACCGCGG
CTCCCACTACTGCCCAAACTGCCAGAAGCGGCGCTAGCTG
>RXA02079
ATGAGCGAAGCTTTTGATGCAACCAAAGTGCGCAAAGCTGTGCTCACGGTGGCGCTGCTT
AACTTCGCTTATTTCTTTGTAGAATTCTTTATTGCATTAAGCGCAGGCTCCGTTTCTCTA
CTGGCTGACAGTGTCGATTTTCTTGAAGACACCTCGATGAACCTGCTCATTTTCATTGCC
CTAGGATGGCCGTTGGCGAGGCGCGCAGTGATGGGCAAACTTATGGCGATTGTGATTCTT
GCACCTGCTGCTTTTGGTGCGTGGGCAGCGATTCAACGGTTTTCCGCACCGCAAGCGCGC
GAAGTGTTTCCGATCATCGTCGCTTCTCTGGGGGCGGTCGTGATCAACGGCGCGAGTGCC
ATCATTATTTCTCGAGTGCGACAACATGGTGGCTCGCTTGGCCAAGCTGCCTTCCTATCC
GCCCGAAATGACGTCCTGATCAAGATTGCCATCATCATGATGGCCTTAATTACCGCATGG
ACGACGTCTGGATGGCCAGATTTGATGCTAGGTTGTTTCATCATTCTGCTCGCACTGCAC
GGCGCTCAGGAGGTGTGGGAAGTCAGTGAGGAAGAACGCCTCGCCTCCAAAGCCCTTGCT
GGGGAAGCCATCGAT
>RXA02079-downstream
TAGGGGAGCAGTATGAGCTTTTC
>RXA02096-upstream
CGCTTCGACGACCTCACCCACAGCGATATCCGCAGGAATGTCATCGCGGTTTTTGATGAG
CCGTTCTTGTAGTGCTCCTCCATACCGCGAGAACATCTCG
>RXA02096
ATGGGTTTGGATGTCAGTGATGAGCAGATCGAACACGCAGCCAGGCTTGCCCAGGCTCAT
GATTTTATCGATCGCCTTCCAAACAAATACGAGGAAGTCATTGGCGAACGCGGCCTGACG
CTTTCTGGTGGTCAACGCCAACGCATCGCCCTCGCACGGGCTTTCCTGGCGCATCCCAAA
GTGTTGGTGCTTGATGATGCCACCTCTGGCATTGATGCCTCCACTGAGGACCGCATTTTC
CAGGCCTTGCGGGAAGAACTGCACGATGTCACCATTTTGATCATCGCGCACCGCCACTCC
ACTTTGGAGCTCGGCGATCGGGTTGGTCTGGTCGAAGATGGACGGGTAACAGCACTGGGA
CCGTTGAGTGAGATGCGTGATCACGCTCGTTTCTCGCATCTGATGGCTCTTGATTTCCAG
GATTCTCAGGATCCGGAATTCACCCTCGACAACGGTTCACTACCCAGCCAAGAGCAATTG
TGGCCGGAGGTCTCCACAGAAAAGCAGTACAAGATTCTTGCGCCTGCCCCTGGTCGAGGC
CGTGGCATGTCCATGCCAGCAACCCCTGAGCTGCTCGCCCAGATTGAGGCGCTGGCAGCA
GCAACGGAAGAAACACGAGTTGATGCCGGGAGGCTACGCACCAGTACCTCCGGTTTCAAA
TTGCTCAGTTTATTCAACCAGGTCCGTTGGCTCGTCGTCGCGGTCATCGCGTTGTTGCTG
GTGGGCGTAGCCGCCGATCTAGCATTTCCAACACTGATGCGCGCAGCCATCGACAACGGT
GTGCAAGCACAAAGCACCTCCACGTTGTGGTGGATCGCCATCGCAGGCAGCGTAGTAGTC
CTTCTGTCCTGGGCCGCCGCCGCGATCAACACGATTATCACGGCACGCACCGGTGAACGG
CTGCTTTACGGCTTGCGTCTGCGCTCATTTGTGCATCTATTGCGCCTGTCCATGAGCTAT
TTCGAACGCACCATGTCCGGCCGCATCATGACGCGCATGACCACCCACATCGACAACCTC
TCGTCCTTGCTCCAATCAGGTCTGGCGCAAACAGTTGTCTCTGTGGGCACGCTCATCGGT
GTGCTCACCATGCTCGCCATCACCGACGCACAACTAGCACTCGTTGCGCTGTCCGTGGTG
CCGATCATCATCCTGCTCACTCTCATTTTCCGACGCATCAGCTCCAGGCTGTACACCGCT
TCACGCGAGCAAGCCAGCCAGGTCAACGCGGTATTCCACGAGTCCATCGCCGGTTTACGC
ACCGCGCAGATGCACCGCATGGAAGACCAAGTCTTTGACAATTATGCGGGCGAAGCA
>RXA02119-upstream
TTCGGTCCGCTCTGGCAAAAATGGCTGGCTGCCACCTCGGCGCAGCAGCTTAAGGGCTGG
GCTTAAATTGCTTGTCGACGCCTAGTGCCACAATGGAGAC
>RXA02119
ATGACCGAAACACTTGTGGTGAATGGCCTAGCAGGCGGCTATGGGCACCGCACATTATTT
AACGATGTGAATCTCACCGTAGCTGCCGGCGATGTCGTGGGCGTTGTCGGCGTCAATGGC
GCTGGTAAATCCACATTTCTAAAAATTCTGCCGGGCGTGGAAAAGCCACTGGCTGGAACT
ATCGCGCTTTCGCCAGCCGATGCTTTTGTGGGCTACTTGCCACAGGAACACACCCGCACG
TCTGGAGAGACGATCGCAGTTTACATTGCTCGTCGAACCGGCTGCCAAGCTCCAACAACT
GCCATGGATGACACCGCCGAAGCGTTTGGTGCGGATCCAGACAACGCTGCCTTGGCCGAT
GCATACGCCGAGGCGCTGGATCGGTGGATGGCCAGTGGCGCAGCCGATTTGGATGAACGC
ATCCCCATCGTGCTCGCTGATTTGGGCTTTGAGCTTCCCACCTCGACGCTGATGGAAGGA
CTTTCAGGCGGGCAGGCAGCCCGCGTCGCGCTGCCGGCGTTACTGTTGTCACGTTTTGAC
ATTGTGCTTCTCGACGAGCCCACCAACGATTTGGATCTCGACGGTCTTGAGCAACTGGAG
AATTTTGTTCACGGGCTTCGCGGGGGAGTCGTACTGGTCAGCCATGATCGTGAGTTTCTT
TCCAGGTGTGTGACCACTGTGCTGGAACTCGATCTGCACCAAAATTCCCACCATGTTTAT
GGCGGTGGATATGATTCCTACCTTGAGGAACGCGCAGTGCTACGCCAGCACGCCCGTGAC
CAATATGAGGAATTTGCGGAAAAGAAGAAGGACCTTGTGGCACGTGCTCGAACGCAGCGT
GAATGGTCTAGTCACGGTGTCCGCAATGCTATTAAACGTGCACGTGACAACGACAAACTT
CGGAACAAAGCCGCTGCGGAATCCAGTGAAAAGCAGGCTCAAAAAGTCCGCCAGATGGAA
AGCCGCATCGCTCGGTTAGAAGAAGTTGAAGAGCCACGTAAAGAATGGAAACTGCAGTTC
AGCGTCGGTAAGGCGTCGCGGTCAAGTTCTGTTGTTTCCACGTTGAATGATGCAAGCTTC
ACCCAAGGCGATTTCACGTTGGGACCAGTATCCATCCAAGTAAATGCTGGCGATCGCATT
GGCATCACAGGACCCAACGGTGCTGGTAAATCCACATTGCTGCGCGGACTATTGGGAAAC
CAAGAACCCACCAGCGGTACTGCCACGATGGGCACGAGCGTGGCGATCGGAGAAATCGAT
CAGGCACGAGCGTTACTTGATCCACAGTTGCCACTGATTTCTGCGTTTGAAAAGCATGTT
GCAGACTTACCGATCAGTGAGGTGCGCACACTGCTCGCGAAATTTGGGCTGAATGATAAT
CATGTGGAAGGGGACGTCGAAAAGCTATCTCCTGGCGAGCGCACGCGCGCCGGACTTGCG
GTGCTACAGGTGCGGGGCGTCAACGTGCTTGTTCTTGATGAGCCCACCAACCACCTTGAC
CTGGAGGCCATCGAGCAATTGGAGCAAGCGTTGGCCTCGTATGATGGTGTGTTGCTGCTG
GTCACGCACGATCGTCGCATGTTGGACGCTGTGCAGACCAATCGTCGTTGGCATGTCGAG
GCTGGCGAAGTTAGGGAGCTA
>RXA02119-downstream
TAACCGTTTCCGTATTGATGCCA
>RXA02220-upstream
GGGCTTTCGCCGCGGAATGGTCCCTCGTCCAGGTCTTTAATTGATGTCTTGACGTGATCT
GGGCGGGCACGCGGCCAATCATGTGAAAGGTCTGTTTTAG
>RXA02220
GTGTCGTCCCCTCTCCCCGCTGCCGTGACATCAAAACCCGCCCACGCGCTTTCCTCTGAT
GAGGTGTTAGAAAATCTCGGGGTCCAGGACACCGGATTGACCTCCGCGGAGGCAACACAG
CGTTTGGAAGCAAACGGGCCAAACGAGGTTCCTCAAACTCCACCTGAAACAGTCTGGCAA
CGGCTATTCCGCCAGGTGAACGATCCAATGATCTACGTTCTCATTGCCGCCGCGGTACTC
ACGGCGTTTCTTGGGCATTGGACAGACACCATCGTGATCGGCGCCGTTGTCATCATCAAC
ATGATGGTTGGGTTCATCCAAGAGGGCAAAGCTGCGGATGCGTTGGCATCGATCCGCAAC
ATGCTCTCCCCGGAATCCGCGGCGTTGCGCGATGGGGTCTTCCACAAAATTGATGCGGCA
GAGCTGGTGGTCGGTGACGTTGTGAAACTATCCGCCGGCGATAAAGTGCCCGCTGACCTG
CGCATGCTCGCCGCCACCAATCTGCACATTGAGGAATCCGCGCTCACCGGCGAGGCGGAA
GCAGTGGTCAAAGGTACTGATCCAGTTGAGGCGGACGCCGGAATCGGCGACCGCACATCC
ATGGCGTTTTCAGGAACGCTGGTGCTCACAGGCAGCGGCACCGGCGTGGTCACCGCCACC
GGTGCAGGCACAGAAATCGGGCACATCACCACCATGCTTGCCGACGTCGACTCCGTGGAT
ACCCCATTGACTCGGTCGATGAAAAAGTTCTCATCGGCGTTAGCAATCGTGTGTGTATTC
CTAGCGATCCTCATGCTGGTGGTTGCCGGTCTAGTCCACCACACACCTTTGGAAGAGCTC
ATTCTTTCCGCCATCGGCTTTGCGGTGGCTGCCATTCCGGAGGGTCTACCTGCGGTTATC
GCCATCACGCTGGCATTGGGTGTGCAAAAGATGGCAGCTCGAAATGCGATTACGCGCCGG
TTGAATTCCGTGGAAACACTTGGCTCTGTCACCACCATCTGCACGGATAAAACCGGCACA
CTCACCCGCAATGAGATGACAGTCGGCGCAATCGCCACCGGTACGAGTCTTTATGACGTC
AGTGGAGCAGGCTACGAAGCTCTCGGGGAAATCCGCTTAAAAGACGGCGAGCAAGTATCC
AAGCAGGATTTCCCAGATCTCTACGGGATGGCGTTGGTGGCAGCGAACGTCAACGACGCC
GAAATTTACGAAGAAGACGGCATGTGGAGGCTTTCCGGCGAACCCACGGAGGGCGGTATT
CGTGCCTTTGCAATGAAAACCAACGCTGAAATCTTGACCCGAAGAGCCGAAGTCCCCTTC
GATTCCGCATAGAAATACATGGCGACGCTTCACACCATCGATGGAGCAAACACCATGCTG
GTCAAGGGCGCTGCCGATCGTTTATTGGATAGAAGTGCACAGCAGCGCAACGGTGAACCA
CTTGACCGGCCGTATTGGGAACAGCTGATCGAGGACCTCGCGTCCCAAGGGCTCCGCGTG
CTGGCTGCGGCATATAAAGAGCTTCCCCACAGCACGTCAACAATTACTCCAGAAGATGTT
GACCAGGGCGAACTCACCTTCCTCGGGCTCTACGGCATCATGGATCCGCCACGCGAAGAA
GTCATCGAAGCCATGAAAGTGGTGCAATCGGCAGGCGTTCGCGTCCGCATGATCACCGGC
GATCACTCCTCCACGGCCCGCGCAATCGCCCGCGAAGTGGGAATCCGCGGCCAGAACGTG
CTCACCGGTGCGGAAATTACTGCGGCTACTGATGAGGAGCTGCAGGGACTCGTCGATAAT
GCTGATCTTTTTGTGCGCACCAGCCCCGAGCACAAGCTGCGCGTCGTGCGCGCACTGCAA
GCTAACGGCGAAGTCGCGTCCATGACGGGCGACGGGGTCAACGATGGGCCAGCGCTAAAA
CAAGCCGACGTCGGCGTCGCCATGGGCATTAAGGGCACCGAAGCCACCAAAGACGCGGCC
GACATCGTGCTTGCCGACGACAATTTCGCCACAATCGCCGGCGCCGTAGAAATGGGTCGC
ACCATCTACGACAACCTGCGCAAAGCCGTCGTCTTCATGCTCCCCACCAACGGCGCCCAA
GGCCTCGTCATTTTCATCGCGATGCTGCTCGGCTGGGAACTGCCCATCACCGCACTTCAA
GTGCTGTGGATCAACCTCATCACCGCGATCACACTGTCCCTGGCGCTGTCCTTCGAGCCG
GCCGAGCCCGGCATCATGAACAGAAAAGCCAGAAACCCCAAGAGGGGGCTTATCGACGCC
CCCTCCGTGCTTCGCATGGTCTATGTCTCCCTGCTGCTCGGCGGAGCAACGTTCTGGGCT
TTCCTTGGCGCGCGCGACGCAGGAATCGACATCGACACCGCCCGCACCATCGCGGTGACC
ACCCTTGCAGTCAGCCAAGTGTTCTACCTTTTAAGCTCCCGATACTTCGAAGTATCCGCG
CTGCGAAAAGAACTGTTCACCACCAACGCGATTTCCTGGCTGTGCATCGCACTCATGCTG
ATCCTGCAAGTGGCCTTTGTCTACGTGCCGTTCATGCAAAGCACCTTCGACACCGCCGCA
CTGACGCTTAGAGATTGGGTCATGCCACTGGTGTTTGGTGTTGTTGTCTTTGCGGTCGTT
GAAACCGAGAAATTGATCAGGCGCCTTAAAGGGTCT
>RXA02220-downstream
TAAGGTTTCAGCCCCTCAAGATA
>RXA02222-upstream
CATGCGCTGAACATCGTCTGTCTACAGCGTTTGGAGAACGGGAAAAAGATGAGGCAGTAC
AAGATTGCAAAAACCTTGAAAAAGTGTATGGCAGCGATGG
>RXA02222
TTGGGTCGACCTCCCCCAGGAGACGTTCATACTCTCCTAGACGATATCGGAGCAGAGGAA
TCTGAAGCAGATAAAGTTCCAATTGAATGGCAAAACGCCCTGACTAAGGCAGACAGGTAT
GCAAACCGGCAACACATGTCTCAGGCACGACTCTATCGCCAATTAACCAGTGATGTTGGA
GAGGGCTTCACTGAAGAAGCTGCCCAATACGCAATCGAAAATGTGAACGCAGACTGGAAC
GCTAACGCCCTAGTAAAAGCAAGAAATTACCAGGAGCGCCAAGCAATGTCAGTAGACGGC
ATTTACAGGCAACTTACTAGTGAACACGGTGAAGGGTTTACCCCAGAGCAGGCACAATAC
GCGATCGACAACCTA
>RXA02222-downstream
TAAGGCATAAAGATCCTAGTATT
>RXA02312-upstream
TTAGCGCCCATTAACGCTTCACATCCTTATATTCCCAAGGAGCACGACCATTTCTGATTC
AGCAGTCCAGGAGAATCACGAACCGCACCTCAAGCGCGGT
>RXA02312
TTGAGCAATAGACACCTTCAGCTCATCGCCATCGGCGGAGCGATCGGTACGGGTCTGTTC
ATGGGGTCCGGCAAGACGATCTCCGTTGCGGGGCCATCAGTAATTTTGGTGTACGCCATT
ATTGGTTTCATGCTTTTCTTCGTCATGCGTGCCATGGGAGAGCTGCTGCTCGCCAATTTG
AATTACAAATCTTTGCGCGATGCGGTCTCTGATATTTTGGGTCCTGGCGCAGGTTTTGTC
ACCGGCTGGACATATTGGTTCTGCTGGATTGCCACAGGCATGGCGGACATCGTGGCGATC
ACTGGATACACCCAATACTGGTGGCCTGAGATCCCATTGTGGCTTCCAGGTGTGCTCACC
ATTGCGTTGCTGTTTGCCCTGAACTTGGCTGCGGTACGACTGTTGGGTGAGATGGAGTTT
TGGTTCGCCATCATCAAAATCGTGGCTATCGTGTCCTTGATCGTCGTGGGACTTTTCATG
GTGGTCACAGCCTTTGAATCACCTAATGGCACCACCGCGCAGTTCAACAACCTCATTGAG
CATGGCGGATTTTTCCCCAACGGCATCACCGGTTTCTTGGCTGGTTTCCAGATCGCTATC
TTTGCGTTCGTCGGGATTGAACTTGCCGGCACTGCAGCTGCAGAGACTGAGAATCCCAGC
AAGACGCTTCCTCGGGCAATCAACTCCATTCCCATCCGCATCGTGGTGTTCTATGTTTTG
GCGTTGGCTGTCATCATGATGGTCACCCCATGGGATCAGGTCCGTGCTGACAACAGCCCA
TTGGTGGAGATGTTCGCGCTGGGAGGAATCCCAGCGGGGGCAGGCATCATTAACTTTGTG
GTCATCACTTCTGCAGCGTCGTCTGCCAACAGTGGTATTTTCTCCACCTCGCGCATGTTG
TATGGATTGTCTTTGGAAGGCGCAGCTCCGAAACGGTGGAGCCGGTTGTCCAAGAACTTG
GTGCCAGCCAGGGGATTGACTTTTTCTGTGATTTGCCTCATTCCAGCGGTGGGTTTGCTG
TACGCTGGCGGCACTGTCATCGAGGCATTCACACTGATCACCACGGTTTCTTCGGTGTTG
TTCATGGTGGTGTGGTCCTACATTTTGGTGGCTTATATCGTCTACCGCCGCAACAGCCCG
GAATTACACAAAAAGTCGATTTTCAAAATGCCTGGCGGCGTGGTCATGGCAGTTGTGGTG
TTGGTGTTCTTCGCAGCGATGTTGGTGGTGCTGTCCCTGGAGCCGGATACCCGTGCAGCG
CTCATCGCGACGCCAGTGTGGTTCATCATTTTGGGTATCGGTTGGTTGTCCATCGGTGGA
GCTAAGGGCGCTAAGCATCGCAGCCAAATAACCTCCCAC
>RXA02312-downstream
TAAAGCTCCTGGGTTAGACTCGA
>RXA02313-upstream
CAGGATGTAACCGAAAAGATCTCAACACTTAAATAAAGTTCTCGATAAAGCCATGTTGGG
TTAACTGCGATGTAGGCATGATGTGGAGATAATAAGGCCC
>RXA02313
ATGCGGGTAGCAATTGTTGCAGAGTCGTTGCTTCCAAATGTCAACGGAGTCACGAACTCG
GTGCTCCGGGTGTTGGAGCATTTGAAAGGCAAGGGACACGACGCGCTCGTCATCGCGCCG
GGTGCCCGGGATTTTGAAGAAGAAATCGGCCACTACCTGGGCTTTGAAATTGTGCGCGTC
CCCACCGTTCGGGTCCCACTGATTGATTCACTGCCCATCGGTGTTCCTCTGCCCTCAGTT
ACCTCTGTGCTGCGCGAGTACAACCCAGACATCATTCACCTGGCATGCCCATTTGTGCTC
GGTGGAGCGGCAGCATTCGCAGCAAGGCAGCTGCGCATCCCAGCAATTGCTATCTATCAA
ACTGATGTGGCAGGGTTCTCCCAGCGCTACCACCTGGCACCGTTGGCCACTGCAAGCTGG
GAATGGATCAAGACGGTCCACAACATGTGCCAGCGGACCCTTGCTGCCTCATCGATGAGC
ATTGACGAGCTGCGTGACCACGGAATTAATGATATTTTCCACTGGGCTCGGGGCGTGGAC
TCCAAGCGTTTCCACCCTGGAAAGCGTTCCGTAGCGCTAGGTAAGTCTTGGGATCCAAGT
GGAGCAAAGAAGATCGTTGGTTTCGTTGGGCGCCTTGCATCCGAAAAGGGCGTGGAGCGC
CTTGCTGGATTATCCGGACGCTCAGACATCCAATTGGTCATCGTCGGTGATGGCCCAGAG
GCCAAGTACCTGCAGGAAATGATGCGGGATGCGATCTTCACAGGAGCTCTCGGCGGCGAG
GAACTAGCCACCACCTACGCATCACTCGATCTGTTTGTGCACCCAGGTGAGTTTGAAAGC
TTCTGCCAGGCGATCCAGGAAGCCCAAGCATCAGGTGTGCCCACCATTGGCCCACGCGCA
GGTGGTCCCATTGATTTGATCAACGAAGGCGTCAAGGGCCTGCTTCTTGATGTTGTAGAT
TTCAAGGAAACCCTCCCCGCTGCAGCCGAATGGATTTTGGACGATTCCCGCCACTCCGAA
ATGTGCGCAGCTGCTTGGGAAGGTGTGAAAGACAAGACCTGGGAAGCTTTGTGCACCCAG
CTTCTCCAGCACTACGCGGATGTAATCGCATTGTCACAGCGCATCCCACTGACATTCTTT
GGCCCTAGCGCTGAAGTAGCAAAGCTTCCACTGTGGGTTGCTCGCGCGCTGGGTGTTCGC
ACCCGCATCAGCATCGAGGCT
>RXA02313-downstream
TAACTCTGCAGAATTAATCCATG
>RXA02344-upstream
AAAGACCCGAGCCGAAGCCCTGGCCTGCGCATACTTCCTTGTCAACGCTCGCTGGGATTA
GGTCTTTTCTGAGCGCTAGCATTTCTCCACTCAAAGGAGC
>RXA02344
ATGCTTAACCGCATGAAAAGTGCGCGGCCAAAATCAGTCGCTCCAAAATCCGGACAAGCT
TTACTCACTCTCGGTGCCCTAGGTGTTGTGTTCGGCGACATCGGCACCAGCCCCCTGTAC
TCACTTCACACTGCATTCAGCATGCAGCACAACAAAGTCGAAGTCACTCAGGAAAATGTG
TACGGCATCATCTCCATGGTGTTGTGGACCATCACTTTGATCGTCACCGTCAAATACGTC
ATGCTGGTCACCCGAGCTGACAACCAAGGACAAGGTGGCATCCTGGCGCTCGTTGCTTTG
CTGAAAAACCGTGGGCACTGGGGAAAATTCGTGGCAGTAGCCGGCATGTTGGGCGCCGCA
TTGTTTTATGGCGATGTGGTGATCAGCCCGGCGATCTCTGTTCTCAGCGCAACAGAAGGC
TTGACGGTTATCTCCCCAAGCTTTGAGCGCTTCATTCTGCCCGTATCTCTCGCAGTTCTG
ATCGCTATTTTTGCAATCCAACCGCTCGGTACAGAAAAAGTCGGCAAAGCCTTCGGCGCC
ATCATGTTGCTGTGGTTTGTCACCCTTGCAGGATTGGGAATTCCGCAAATCATCGGGCAC
CCAGAAATCTTGCAGAGCTTGTCTGCACATTGGGCCCTGCGCTTGATTGTGGCTGAGCCT
TTCCAAGCATTTGTGCTG
>RXA02348
CCAATCAGAGTGGGGTGGTTTTGCGTCGTCATGCCTGCTTTAATCTTGACGTATTTGGGG
CAGGGCGCCTTGGTGATCAACCAGCCTGAAGCGGTGCGCAACCCCATGTTTTATCTCGCG
CCGGAAGGTCTGCGGATTCCGTTGGTTATTTTGGCGACCATCGCTACGGTGATCGCATCG
CAGGCCGTGATTTCTGGTGCGTATTCATTGACCAAGCAGGCCGTGAATTTGAAACTGCTG
CCACGCATGGTGATCCGGCATACCTCCCGCAAAGAGGAAGGCCAGATCTATATGCCACTG
GTTAATGGATTGCTGTTTGTATCCGTGATGGTTGTGGTGCTGGTATTCCGATCCTCTGAA
AGCCTCGCCAGCGCGTACGGACTTGCAGTGACCGGAACCTTGGTGCTGGTCAGCGTCCTG
TATCTGATCTATGTTCACACCACATGGTGGAAAACAGCGCTGTTCATTGTGCTCATCGGT
ATTCCAGAAGTACTTCTATTCGCCTCGAACACCACGAAAATTCACGACGGTGGCTGGCTT
CCACTACTTATTGCGGCCGTGCTCATCGTGGTGATGCGGACCTGGGAGTGGGGAAGTGAC
CGCGTCAATCAGGAACGCGCAGAGCTGGAACTTCCCATGGATAAGTTCTTGGAGAAACTC
GATCAGCCACACAATATTGGTCTGCGTAAAGTTGCCGAAGTGGCAGTATTTCCACATGGC
ACCAGCGATACTGTCCCGTTGTCATTGGTTCGCTGCGTGAAAGACCTCAAGCTTTTATAC
CGAGAGATCGTGATCGTTCGAATCGTCCAAGAACACGTTCCGCACGTGCCAGCAGAGGAA
CGCGCGGAAATGGAAGTGCTCCATCACGCCCCGATCAGAGTCGTGCGAGTTGATCTGCAC
CTTGGTTATTTTGATGAGCAGAACGTGCCTGAGCATCTCCATGCCATTGACCCAACATGG
GATAACGCCACCTACTTCCTGTCTGCCCTGACTCTTGGGAGCAGGTTGCCTGGAAAGATT
GCTGGCTGGCGTGATCGTTTGTATCTTTCGATGGAACGTAATCAGGCATCTCGAACTGAG
TCTTTCAAATTGCAACCAAGCAAAACCATCACGGTTGGAACAGAGCTGCACCTT
>RXA02348-downstream
TAATCAGGCAGTTGCTGGCCAAC
>RXA02353
ATGGCACTGCTGATCCTCGCCGGTCTGCAAATGATCCCGAAGGAAACCTACGAAGCAGCC
CGCGTCGATGGCGCAACCGCGTGGCAGCAATTCACCAAGATCACCCTCCCGGTGGTGCGC
GCAGCTTTGATGGTGGCAGTACTCTTCCGCACCCTCGATGCGCTACGCATGTATGACCTC
CCCGTCATCATGATCTCCAGCTCCTCCAACTCCCCCACCGCTGTTATCTCCCAGCTGGTT
GTGGAAGACATGCGCCAAAACAACTTCAACTCCGCTTCCGCCCTTTCCACACTGATCTTC
CTGCTGATCTTCTTCGTGGCGTTCATCATGATCCGATTCCTCGGCGCAGATGTTTCGGGC
CAACGCGGAATAAAGAAAAAGAAACTGGGCGGAACGAAGGATGAGAAACCCACCGCTAAG
GATGCTGTTGTAAAGGCCGATTCTGCTGTGAAGGAAGCCGCTAAGCCA
>RXA02353-downstream
TGACTAAACGAACAAAAGGACTC
>RXA02354-upstream
GAATAAAGAAAAAGAAACTGGGCGGAACCAAGGATGAGAAACCCACCGCTAAGGATGCTG
TTGTAAAGGCCGATTCTGCTGTGAAGGAAGCCGCTAAGCC
>RXA02354
ATGACTAAACGAACAAAAGGACTCATCCTCAACTACGCCGGAGTGGTGTTCATCCTCTTC
TGGGGACTAGCTCCCTTCTAGTGGATGGTTATCACCGCACTGCGCGATTCCAAGCACACC
TTTGACACCACCCCATGGCCAACGCACGTCACCTTGGATAACTTCCGGGACGCACTGGCC
ACCGACAAAGGCAACAACTTCCTCGCAGCCATTGGCAACTCACTGGTCATCAGCGTCACC
ACAACAGCGATCGCTGTTCTCGTGGGAGTGTTCACCGCCTACGCTCTAGCCCGACTGGAA
TTCCCGGGCAAAGGCATTGTCACCGGCATCATCTTGGCAGCCTCCATGTTCCCCGGCATC
GCCCTGGTCACTCCGCTGTTCCAGCTCTTCGGTGACCTCAACTGGATCGGCACCTACCAA
GCGCTGATTATCCCGAACATTTCCTTGGCGCTACCTCTGACGATCTACACGCTCGTATCC
TTCTTCAGGCAACTGCCCTGGGAACTCGAAGAATCAGCACGTGTCGACGGCGCCACACGT
GGCCAAGCGTTCCGCATGATGCTGCTTCCTCTAGCAGCGCCCGCACTATTTACCACCGCG
ATCCTCGCATTCATTGCAACGTGGAACGAATTCATGCTGGGCCGCCAACTATCCAACACC
TCCACAGAGCCAGTGACCGTTGCGATCGCAAGGTTCACCGGACCAAGCTCCTTCGAATAC
CCCTACGCCTCTGTCATGGCAGCGGGAGCTTTGGTGACCATCCCACTGATCATCATGGTT
CTCATCTTC
>RXA02394-upstream
TGTTGATGAATCAGAAGAACCTCGAGATTTGGACGAGCTAGAGGCCCAAAGCGCTATAGA
TTCTGCAAGTTCAGCGGAAGGTAGGAACTAAT
>RXA02394
ATGTTGTCGCCAGCAGCTGTAGCAGCTTTAATTCTTGTCATCGGCATTGTGGTGCTCATC
ATCGCATCAGTGCCCGTTGCCATTGCCATCGGTTTGCCATCACTTTTTGCCGCGATGGCC
GTGCTTGGCCCAGAAAACGCCGCGCAGGCCGTCGCGCAGCGCATGTTTACCGGCACAAAC
TCCTTTACACTCCTTGCCATTCCGTTCTTCGTGTTGGCGGGTTTGCTGATGAACTCGGGT
GGTATTGCCACGCGGCTTATCGACGCCGCGAAGGTGCTTGTCGGCCGCATGCCTGCCTCC
ATCGCCAATACGAATATCGCAGCAAATGGTCTCTTCGGAGCAGTTTCAGGGGCAGCGGTA
GCATCAGCTTCTGCCGTGGGAACCGTCATGACACCAAAAATGAAGGAAGAGGGCTACTCG
CGCGCTTACGCAGCGGCCGTCAACGTGGCTTCAGCACCTGCGGGCATGCTGATCCCGCCA
TCAAACACTTTTATTGTGTATTCCTTGGTGTCCTCGACATCAATTGCAGCACTATTTATG
GCCGGTGTTGGACCCGGTCTGCTCTGGATTCTGGCCTGTGTCATCGTGGGAACTTGGTTA
GCGCGAAAGGAAAACTACAAGCGCGAGCAGATTCATCCAACATTCAAGCAGTCGCTCGTT
GTGCTGTGGAGGGCGCTGCCTTCACTGCTCATGATCGTCATTGTTGTTGGAGGTATCTTG
CTGGGCTGGTTCACTCCAACTGAATCCGCTGCTATTGCTGTAGTGTACTGCCTGGTCTTG
GGCTTTATTTACCGCACAATCAAGGTGGGAGATGTGGCAGATATTTTGCTCAAGGCAACT
CGCACCACATCAATTGTCATGTTGCTCATTGCAGTTTCTGCAGCACTGTCGTGGGTGATG
GCCTTTGCCAAGATCCCTCAGATGATCTCTGATGCGCTTCTTTCGGTATCCGATTCCAAG
GTTGTCATGTTGTTGATCATGATGTTCATCCTGTTACTCATGGGTAGCGTAATGGACCCA
ACACCAGCAATTTTGATCTTCGTCCGGATCTTCGTTCCAGTGGTTACCGAACTTGGTGTG
GACCCAGTCCACTTCGGTGCGATGGTGGTAATGAACCTGTCCGTGGGCGTGATTACCCCA
CCAGTAGGCAACGTGTTGTTCGTTGGTTCGCAAGTGGCAGGGCTGCGTGTGGAAACTGTG
ATCAGACGACTGTGGGCGTATCTCATTGCCATTATTGTTGCGCTGTTCGTGGTTGTTTTC
GTACCGCAGATCTCTATCTGGCTGCCGACAACAATGGGATTGATGGGAGGC
>RXA02394-downstream
TAAACCTCCAGCCATCAGCTAAG
>RXA02402-upstream
CACTACTGCGTTAAGGTATGAAAGTTCGCACACCAGCGATTTAATTCTGTGCCCACCACT
AGCACGAGCATTTCAGTTTTAACTTTCTTGGAGTTTTCTA
>RXA02402
GTGTCCAAAACAGAAGAAGGCCGTTCAGCGGCGATAATTATTTACGCGTTTCCAACTTTG
ATTGTGCTGGGCGCGATCATTGCGTTTATCTTCCCGGAACCATTCATTCCGCTGACAAAC
TACATTAATATCTTCCTCACGATCATCATGTTCACCATGGGTTTGACCTTGACGGTGCCC
GATTTTCAGATGGTGCTTAAACGTCCACTGCCTATCTTGATCGGTGTAGTAGCGCAGTTT
GTCATCATGGCATTCCTGGCGATCGTGGTTGCGAAAATGTTCAACCTCAACCCAGCACTC
GCCGTTGGCCTTCTCATGCTGGGATCGGTTCCGGGTGGCACCTCCTCCAATGTGATTGCG
TTTCTGGCCCGAGGAGATGTCGCGCTATCGGTCACCATGACCTCTGTGTCCACGATTGTT
TCCCCAATGATGACGCCTTTCCTCATGCTCATGCTGGCAGGTACTGAAACCGCCGTCGAT
GGTGGAGGCATGGCGTGGACTTTGGTACAAACAGTGCTGCTGCCTGTGATCATCGGCCTA
GTTCTGCGTGTCTTCTTGAACAAGTGGATCGACAAGATTTTGCCGATCCTTGCTTATGTC
TCCATCCTCGGTATCGGTGGCGTGGTGTTCGGCGCAGTCGCAGCCAACGCGGAACGACTC
GTGTCTGTCGGACTCATCGTGTTCGTTGCAGTTATCGTGCACAACGTACTTGGATACGTT
GTGGGATACCTCACCGGCCGTGTA
>RXA02422-upstream
CTTAAACGTCACCTTATTTATGCATTATGTTGGTTTCAGACTCGAACAATTCAATTAGAA
AACACTAATCGGACATTTAGGTCACATAACATTTCCGCTC
>RXA02422
GTGTCCACATTAATTTCTGAACCCGAGGTGGATAAGCTACGTAAACGTGCCAAGAGATCA
AGGCGGACAGAATGGTGGCTTGCCGCCGCACTTCTTGCCCCAAACTTGCTTCTCTTGGCC
ATCTTTACGTATCGGCCACTGTTAGATAACTTCCGGTTGTCCTTTTTCAACTGGAACATT
TCCTCGCCCACATCAAGCTTCATTGGGTTTGATAACTACGTTGAGTTCTTCACTCGTAGT
GACACTCTCCAAGTTGTTTTAAACACCGTCATCTTCACGGCATGTGCTGTGATCGGATCG
ATGGTGCTCGGTTTGCTCGTGGCGATGTTGTTGGATCAGAAGCTTTTCGGCCGTAACTTT
GTGCGTTCCATGGTGTTTGCCCCGTTTGTGATTTCCGGTGCTGCCATTGGTGGTGCTTTC
CAGTTCGTTTTTGAC
>RXA02438-upstream
CAGGTTGGAACCCTGACTGGTTCATGTTCTTCCTCGGCGGCACCCTACTTCTGGCTGTTT
TGCTCAATCACCGATTCGAGCGTTTCAACAAGGAGCGATC
>RXA02438
ATGACAGACCTCATTCAACTCCGCGAAGTATCCAAAAAATACGGTGGTTTCCAGGCCCTC
AACGACATCAATTTGAACGTCCGCGCAGGCGAAGTCACCTGTGTTCTGGGTGACAACGGC
GCCGGAAAATCCACCCTCATCAAGATTCTCTCCGGCCTGCATCCCGGCACCTCCGGCGAA
GTAATCGTGGCCGGCGATGTAGTGAATTTTGGATCCCCCCGCGACGCCCTCGACGCCGGA
ATCGCCACCGTCTACCAAGACCTAGCAGTGGTCGGGCAGATGAGTGTGTGGCGCAACTTC
TTCCTCGGCCAGGAACTCACCGGCCGATTTGGCGTTCTGAAACAAGAAGAAATGCGCCGC
ATCACCGACGAACAACTCCGCGAAATGGGCATCGAACTCCGCGATGTCGACGTCCCTGTG
GCCTCCCTTTCAGGTGGTCAACGCCAAGTTGTCGCCATCGCCCGGGCCATCTACTTCGGC
GCGCGCGTCCTCATTTTGGACGAGGCCACCGCAGCGGTGGGCGTGAAACAATCTGGCATG
GTGCTGCGCTTTATTGCCGGAGCACGCGACCGGGGGATCGGCGTCATTTTCATGACGCAC
AACCCCCACCACGCCTACCTTGTCGGTGATCACTTCATCCTGCTCAACTTAGGCAAGCAG
GTCATGGACAAATGGCGCGCAGAAGTCGAGGTGGAAGAAGTCACCCTCGGCATGTCCGGC
GGCGGCGAGCTCGACTCACTCAGCCACGAATTGAAGCGT
>RXA02438-downstream
TAACCTAGTTCTTCTTTTCGCTC
>RXA02439-upstream
GCACCACTGTTGGTGGCGGACGACCCGTGTACACAGGACCAGCCATTGTGGATGCCACCA
ACGTTGATGTCATTGCTGAAGCCGTTGGGGAGGGTCTGCG
>RXA02439
ATGACAAAAATCAAGAGTGGGGAGGCGTCGACAAGCATTGTTGAGCGCGCCTTAAAGCGC
CCCGAACTGACCAGCGTGCTTGGCGGCGTGCTTGTTTTTACGCTGTTTATGGTGGTCGCG
GCGGCATTTAGGTCATGGGATTCGATGGCGACCGTGCTGTATGCGAGTTCCACGATCGGC
ATCATGGCGGTTGCCGTGGGCGTGCTGATGATCGCTGATGAATTCGACCTGTCGACCGGC
GTTGCCGTGACAACTGCAGCGCTGGCGGCCTCGATGTTTAGCTATAACCTGTGGCTGAAC
ACCTGGGTGGGCGCGCTGATTGCATTGGTGATTTCGCTGGCCATCGGCTTTTTCAACGGC
TTTTTGGTAGTGAAAACCAAGATTGGATCCTTCCTGATCACCCTTGCCACTTTCCTTATG
CTGCAGGGTATTAATCTGGCGGTCACCAAGCTGATTTCCGGCACCGTGGCCACGCCAACC
ATCGCGGATATGGAAGGTTTTCCTTCAGGGCGTGCGGTGTTTGCCAGCTCGATTCCCATC
TTTGGTGTGAATATTCGCATCACTGTTTTTTGGTGGCTGCTGTTTGTTATCGTCGGCACT
TTTGTGTTGTTTAAGACGCGCATCGGCAACTGGATTTTTGCGGTCGGTGGCGATGAAGAG
GCAGCTCGCGGAGTCGGGGTTCCCGTGCGTGGCGTGAAAATCGGCCTGTTCATGTTCGTT
GGTTTTGCCGCCTGGTTTGTGGGCATGCACAACCTGTTCCTCTTTGATTCGATTCAGGCT
GGTCAAGGCGTGGGTAATGAGTTCCTCTACATCATCGCTGCGGTGATCGGAGGCATCTCC
ATGACTGGTGGGCGCGGAACAGTGGTGGGCACAATGATTGGTGCACTCATCTTTGGAATG
ACCAACCAAGGCATTGTTTATGCAGGTTGGAACCCTGACTGGTTCATGTTCTTCCTCGGC
GGCACCCTACTTCTGGCTGTTTTGCTCAATCACCGATTCGAGGGTTTCAACAAGGAGCGA
TCA
>RXA02439-downstream
TGACAGACCTCATTCAACTCCGC
>RXA02441-upstream
CCGGCGACCAGGGCGCGGGAGATGAATGAAACGTCAAAGGCACTATGAGGGCGTCAGTA
AAAAACTTCATTTGAAAATGATAACCGTTATCATTAAGGA
>RXA02441
ATGGCAGAACTGAGCGTCCGGAATCTCACATGCACATACGGCAATCACATCGCGCTCAAC
AACATCACGGCACGCTTCCCAACCGGAAAAATAACTGCCCTCATCGGCAGCAACGGCTCC
GGAAAATCCACACTGTTGGAAACTTTGGCGGGCATGCTGGCACCCCGCAGCGGAAGCATT
AACAACGTTGTGCCAGAAATCGCGTTCGTCCCCCAACGCAGGCACGTCTCGGATAATTTG
CCCATCAGGATCAGACAAACAGTCAGCATGGGGCGATGGTCAGCCAAGAAAAACTGGCAA
CGACTCACTGCCGCAGATTGCAACATCGTGGACAGCTGGCTCGACCGGCTCGAAATCTCC
GGCCTCGCCGACCGCCCCCTCGGCGAAGTATCAGGCGGGCAGCGCCAACGCGCCCTCATA
GCGCAAGGTTTAGCGCAACAGGCGCCGTTATTGCTTCTCGACGAACCCCTCGCCGCCGTG
GACTCCCACGCGGCAAGTCTTATCGAAGATGTCATTAACCAACAACGGAACCAAGGAACC
ACAATTATTCTTGCGACTCACGATCTTGATCAAGCACATCAAGCAGATCAGATTATCGCC
TTGGAAAAAGGAATCATAAAGCCACAGCGCAAAGCCACTGAATCAATAAAGAAGCGT
>RXA02441-downstream
TAATAAAGTTTGACTTGTGCCTC
>RXA02442-upstream
GCGGTGATGTTGTTGAGCGCGATGTGATTGCGGTATGTGCATGTGAGATTCCGGACGCTG
AGTTCTGCCATTCCTTAATGATAACGGTTATCATTTTCAA
>RXA02442
ATGAAGTTTTTTACTGACGCCCTCATAGTGCCTTTTGACGTTTCATTCATCTCCCGCGCC
CTGGTCGCCGGATGCCTGGCCGCAATTTTATGCTCACTCATTGGAACGTGGGTTATTTTG
CGCAGGCTAACCTTTTTCGGCGACGCTATGTCGCACGGCTTGCTCCCCGGAGTAGCCACG
GCATCACTATTGGGCGGAAATCTCATGTTCGGCGCAGCAATCAGCGGATTAATCATGTCA
GCCGGAGTGGTGTGGACCAGGAGAAAATCCAGCCTCTCGCAAGACGTCAGCATTGGCCTG
CAATTTATTACCATGCTTTCCCTCGGCGTGGTTATTGTGTCCCACTCCGATTCCCACGCC
GTAGACCTCACCAGTTTCCTTTTTGGAGACATTCTTGGCGTGCGACCCTCGGATATATTC
ATCATCGCCATTGCAACAGTGTTGGGTGGATTGACTATTTTTGTCTTCCACCGACAGTTC
ACTGCACTCGCTTTCGACGAGCGTAAAGCTCACACCTTAGGACTCAATCCCCGCTTTGCA
CACCTAGTCATGCTGGCACTGATCGCATTAGCTACGGTGGTGTGGTTTGAGGTGGTGGGA
ACGCTTTTAGTGTTTGGACTTGTCATTGGTCCGCCCGCCACGGCTGGACTTTTAGTGCAA
GACAAAGCAAGTATTTGACTGATCATGATCGTCGCGTCGCTTCTTGGATGCGCGGAAATT
TACCTCGGGCTTTTAATCAGCTGGCACGCAAGCACTGCCGCGGGAGCCACTATCACTTTG
TTAAGTGCTGCGATATTTTTTGCCACCTTATTGACAAAGAGTGGCATTAGTAGGTTAAAC
TTCACCGCG
>RXA02442-downstream
TGATACTGAAAGACATTTTCAAT
>RXA02447
TGGGTGTGGCTGGCGGAAATCTTCCCAGTCCGAATGAAGGGTATCGGCACCGGTATTTCG
GTATTCTGCGGTTGGGGCATCAATGGGGTCCTAGCGTTGTTCTTCCCAGCACTGGTCTCC
GGCGTGGGTATCACCTTCTCCTTCCTTATCTTGGCAGTCGTCGGAGTCATTGCCCTGGCG
TTCGTCACCAAGTTTGTTCCTGAAACGGGTGGCCGCTCACTTGAAGAACTCGATCACGCA
GCATTCAGCGGCCAGATCTTCAAGAAGGCT
>RXA02447-downstream
TAAACCCCCTCCGATGTCTTTGG
>RXA02451-upstream
GATCAACTTAAGCCTCTAGCTATTTTCAACTGTGTTTCAGTTGCGGGATCGTTGGGTGCC
TAATTGGAGTTGTGCTTTTAGGTGGAGGATCATAGAGGTT
>RXA02451
ATGAACACCGACACAACTCAAGACGGTGTGAGTCCTGAACCTTCCGACCCCCACCTAGGG
TCTGAAGTGGCGGAAACTCACCGCGAAAAGAAATTCTTCGGCCAGCCTTGGGGGCTGGCA
AATCTCTTCGGCGTGGAGATGTGGGAGCGATTCAGCTTCTACGGCATGCAGTCCATCCTT
GCTTTCTATCTGTACTACTCCGTCACCGATGGCGGACTTGGTATGAATCAGACAGCTGCA
CTGTCCATTGTGGGCGCCTACGGCGGCTTCGTCTACATGACCTCCCTCGTGGCTTCGTTC
ATTGCAGACCGAGTATTGGGCTCTGAACGTACACTCTTGTACTCCGCGATCATCGTCATG
CTGGGCCACATTGCCCTGGCCTTGATTCCGGGATATACGGGACTGTCCATCGGCTTGGTC
CTCATCGGCCTTGGCTCAGGTGGCGTGAAGACGGCAGCGGAGGTTGTGCTGGGCCAGCTG
TACTCACGCACGGACACGCGTCGAGACGCAGGCTTCTCCATCTTCTACATGGGCGTCAAC
CTCGGTGGCCTCTTTGGCCCGCTGATCACCAACGCTCTGTGGGGATGGGGAGGATTCCAC
TGGGGCTTCGGTATCGCCGCAGTCGGCATGGCTTTGGGTCTCATCCAATACGTGGCGATG
CGTAAAACCACGATCGGTGCGGCAGGCCATACTGTTCCTAACCCACTGCCTAAGAATGAA
TATGCGCGCTGGATTATCGGTGCAGTCGTGGTTGTCGCAGCAGTTGTCGCTGTCATCGCA
ACGGGCATCATCAAGCTGGAATGGCTGTCCAACATCACCGCAGCGATCGCACTGATTGCG
GCTATTGCTCTGCTTGCTCAGATGTACGTTTCCCCACTGACCACCGCAGCGGAAAAGTCC
CGCTTGTTGGGATTCATCCGGATGTTCATCGGTGGCGTGCTTTTCTTCGCGATCTTCCAA
ACCCAGTTCACGGTCCTCGCGGTTTACTCCGACACCCGCCTGGACCGTAACTTCTTCGGC
ATTGATCTTCCTCCAGGATTGATCAACTCCTTCAACCCAATCTTCATCATCATCTTCTCC
GGAATCTTTGCCACCTTGTGGACAAAACTCGGAGCAAAGCAGTGGTCTACTGCAGTGAAG
TTCGGTGTCGCCAACATTGTCATTGGTTGCGCGCTGTTCTTCTTCCTGCCGTTCGCCGGC
GGTGCAGAGAACTCTACCCCAATGGCACTGATCATTTGGGTCTACTTCCTCTTCACCATC
GCTGAGCTTCTGCTCTCCCCTGTCGGCAACTCACTTGCAACCAAGGTCGCACCCGAGGCA
TTCCAGTCCCGCATGTTCGCCGTGTGGCTGATGGCTGTCTCCATGGGTACGTCCCTGTCC
GGCACCCTGGGTGGTTACTACGATCCAACCGATGCAGGATCTGAAAAGGTCTTCTTCATT
ACCGTTGGCGTTGCAGCCATCGTTCTTGGTGCAATCGTCATAGCAGCCAAGGGCTGGGTG
CTGAAGAAGTTCATCGACGTCCGA
>RXA02451-downstream
TAGGCCTCACAAAGCCTCAAAAC
>RXA02491-upstream
TTTCGTATGCTGACATGGTGTCGCTTCAACTGCGTTGCTTTAGTGCCCTTTAGTATATAG
AGACGTCCCGCTGCTTTCTTCGGCGATCTAGAATGTGGGC
>RXA02491
ATGGGCGTAGCTATGATTTCCATGCACACCTCTCCATTGCAGCAGCCCGGAACTGGTGAT
TCAGGCGGGATGAACGTCTACATTCTTTCGACCGCGACTGAGCTAGGGAAACAGGGTATC
GAGGTCGATATTTACACTCGTGCCACGAGGCCTTCTCAGGGTGAGATGGTGAGAGTAGCT
GAGAATTTGCGGGTGATTAATATCGCTGCGGGGCCGTATGAGGGGGTTTCCAAAGAGGAG
GTTCCTACTCAGTTGGCGGCGTTTACCGGCGGAATGTTGTCGTTTACGCGCCGGGAGAAG
GTTACTTATGATCTGATCCATTCTCACTATTGGCTGTCTGGTCAGGTGGGGTGGTTGCTG
CGCGATTTGTGGCGGATTCCCCTTATTCATACGGCACACACTTTGGCGGCGGTGAAGAAT
TCTTATCGGGATGATTCGGACACTCCGGAGTCGGAGGCGCGTCGCATTTGTGAGCAGCAG
CTGGTGGATAACGCTGACGTGTTGGCGGTGAACACTCAGGAGGAGATGCAGGATTTGATG
CATCACTACGATGCGGATCCGGATGGGATTTGTGTGGTGTCACCGGGTGCGGACGTGGAA
CTTTATAGCCCTGGAAATGATCGCGCGACGGAACGTTCCCGTCGTGAGCTGGGCATTCCG
CTGCACACAAAGGTAGTGGCTTTTGTGGGTCGGTTGCAGCCGTTTAAGGGCCCGCAGGTG
CTGATCAAGGCGGTTGCGGCGTTGTTTGATCGCGATCCGGACCGAAATGTGCGCGTCATT
ATTTGTGGCGGCGCTTCTGGTCGGAATGCGACACCGGATACCTATAGGCATATGGCAGAG
GAACTGGGCGTCGAAAAGCGAATTCGCTTTTTGGACCCGCGCCCGCGGAGCGAGCTAGTG
GCCGTGTATCGGGCGGCGGACATCGTGGCCGTGCCAAGTTTTAATGAGTCCTTCGGAGTC
GTCGCCATGGAGGCGCAAGCCAGCGGCACACCGGTCATTGCGGCCCGGGTTGGCGGCCTG
CCCATCGCAGTCGCGGAAGGGGAGACGGGATTGCTTGTCGACGGCCACTCCCCGCATGCC
TGGGCCGACGCGTTAGCCACACTCTTGGAGGATGACGAAACGCGCATCAGAATGGGTGAA
GACGCCGTCGAACACGCCAGAACATTCTCCTGGGCGGGCACCGCCGCACAGCTATCGTCG
CTGTACAACGACGGTATTGCCAACGAAAATGTCGACGGTGAAACGCATCACGGC
>RXA02491-downstream
TAAGTAAACGCGCGTCGTGGAAC
>RXA02507-upstream
ATTACCCACAATTTCAATCGGTTTATACAACCAGCCTCTAACTGGCAACAGGACTGCAGA
CAGAAACTGTTGCTGGAACCTTCGATGAACAGGATCGACA
>RXA02507
ATGAGCGAACAACTTCAGGGTGTAACTCACTCCGAATCAACTCCGGGCAAGACGCCCAAG
CGAGCAGCACTATCCAGCTGGATCGGCTCAGCTCTCGAATACTACGACTTCGCTGTTTAC
GGAACCGCTGCAGCGCTGGTTCTTAACCACCTCTTCTTCCCAGCTGATACTTCACCAGGC
ATCGCAATTTTGGCTGCGATGGGTACCGTGGGTGTTGCTTATGTGGTTCGCCGTCTTGGT
GCGCTGATCATGGGTCCATTAGGTGACCGTTACGGACGTAAATTTGTCCTCATGCTGTGC
GTCTTCCTGATTGGAGCATCCACTTTCGCAGTTGGCTGCTTGCCAACATTTGATCAGGTC
GGTTACTTGGCTCCGGCACTGTTGGTGCTGTGCCGTGTGATCCAGGGACTGTCTGCATCC
GGTGAGCAGTCCAGTGCGATTTCCGTTTCTTTGGAGCACGCCGATGAGCGTCACCGCGCA
TTTACTGCTAGGTGGAGTCTTCACGGAACCCAGTTCGGTACCTTGCTGGCAACCGGAGTA
TTTATCGCATTCACCTTGTTCCTGAGTGAAGATGGTCTAATGTCATGGGGTTGGCGCGTT
CCGTTCTGGCTGTCCGCTGCTGTTGTTTTGGTTGGTTTCCTCATCCGTCGTGGAGTGGAA
GAGCGACCAGCATTCCGTGAAAACAAGGAAGCAGTTGCAGGCGCAGCATCTCCACTGGCG
ATGACCTTGCGTTACGACAAGGCGGCGGTTGCTCGCGTTGCTATTGCTGCGATGATCAAC
TCCGTGAACATTGTGTTTACTGTGTGGGCACTGTCGTTCGCCACCAACATTGTTGGCCTG
GATCGTTCAACTGTTTTGCTGGTTCCAGTTGTTGCGAACTTGGTTGCACTGATTGCGATT
CCTTTGTCCGGCATGCTGGCTGACCGCATTGGTCGCCGACCAGTGTTCATCATGGGTGCC
ATTGGTGGTGGCCTGGCCATGAACGGTTACCTGGGAGCTATCTACTCCGGCAATTGGACC
ATGATCTTCTTCATGGGCGTGTTGATGTCTGGTCTGCTGTACTCCATGGGTAATGCCGTG
TGGCCAGCGTTCTACGCAGAAATGTTCCCAACCTCTGTGCGTGTCACCGGCTTGGCTCTT
GGAACTCAGATTGGTTTCGCAGTCTCTGGTGGTTTCGTCCCAGTTATCGCATCCGCGCTT
GCTGGTGATCAGGGTGACCAGTGGATGAAGGTGTCCATCTTCGTTGGTGTTGTTTGTGTG
ATTTCTGCACTGGTTGCCATGACCGCTAAGGAAACCAAGGCTCTGACTCTGGATGAGATC
GATGCTCTGCACACTGCTGGTGGTGAGGCCGCAGACCTGGCAGCCGCAAGCAAAGCCTCC
GAGGCCCAACTCGCGGCTCAG
>RXA02507-downstream
TAAAACCAAAAGGAATCTTTGAC
>RXA02515-upstream
GTGGCTAAGCACAGTTAGTTGGCGAAGCTGGGCGGCAGAAAAACCGGCCGAGCTAATACT
TCAGTTTAAAATTGGCTTCAACCCTGAAAGATTGTGACAG
>RXA02515
ATGAGCAGTCTTGAAATGGGTAACCTGCACGCACAGGTCCTGCCGTCCGATGAGTCCGCT
GAGCCTAAGGAAATCCTCAAGGGCGTCAACCTCACCATCAACTCTGGTGAGATCCACGCC
ATCATGGGCCCTAACGGTTGGGGCAAGTCCACTCTTGGTTACACCCTTGGTGGACACCCA
CGCTACGACGTAACCGCAGGCGAGGTCCTCCTCGACGGCGAGAACATCCTGGAGATGGAA
GTTGATGAGCGTGCAGGCGCTGGTCTCTTCCTGGCCATGGAGTATCCAACTGAAATCCCT
GGCGTTTCCGTTGCTAACTTCCTGCGTTCCGCAGCGACCGCAATCCGCGGCGAGGCTCCT
AAGCTTCGCGAGTGGGTTAAGGAAGTCGGCACCGCTCAGGAAGCTCTGGCAATTGACCCT
GAGTTCTCCAACGGCTCAGTCAACGAAGGTTTCTCCGGTGGCGAGAAGAAGCGCCACGAG
GTTCTGCAGCTTGATCTGCTGAAGCCAAAGTTCGCGATCATGGATGAGACCGACTCCGGC
CTTGACGTGGATGCACTGCGCATTGTTTCCGAGGGCATCAACTCCTACAAGCAGGAGACC
GAAGGTGGCATCTTGATGATCACCCACTAGAAGCGCATCCTCAACTACGTTAAGCCTGAC
TTCATTCACGTTTTCGCGAATGGCCAGATTGTGACCACCGGTGGCGCTGAGCTTGCTGAC
AAGCTCGAGGCTGACGGCTACGACCAGTTCATCAAG
>RXA02515-downstream
TAACATGTCCGATTTCCTCAATG
>RXA02562-upstream
CGGGTGCGCTGAGGGTGAGGTTGGGGCGACGAGGCGTGCATGGACTTTAGGTTAGGTTAT
TGAGCAGATTTATTTGGGCTTTTGTCTAGGGTGGGGAGCT
>RXA02562
ATGTTCTTGACAAAGGTTTCGCTGCTTGATCATCCGGAGTCATTGCCGGGGTATTTATCG
AGCCTGGCGATCGTGGAATATCTGCATGAACAGCCGTTGGAGTTTCGTGCACCGATTACT
GTGATTACTGGTGAAAATGGGGTGGGTAAATCCACGTTGGTTGAGGCTTTGGCGGTGGGG
ATGCGCCTTAATCCGTCTGGTGGCTCTAGGCATGCAAACTTTGGCAGGGAAGGCGATATT
GTGTCGTCGCTTCATCAGTCGTTGAAGTTGGTGCGGAGAGAAAACCCTCGGGATGCGTTC
TTTTTTCGGGGTGAGACGATGTATAACGTGGCTTCCTATTATGAGGAGTTAATGGGGGAA
AAGAACATGCATGATCTTCACAAGATGAGCCATGGCGAATCGGTATTTGCGGTGATTGAT
CGGCGTTTTAACAATCAAGGATTTTTTGTTTTGGACGAGCCTGAGGCAGGCCTTTCCATG
CTGAGGCAGTTGGAGTTGTTGGGAAAGTTGGGCAACCTTGCTCGAGGTGGTGCGCAGATC
ATCATGGCTACGCACTCTCCAATATTGTTGGCTATTCCGGGGGCAGAGATCCTTGAAATT
ACATCTTCGGGTGTTGCAAAGGTGAATTTTGAGGATGCGGAGGCTGTTCGTGCGGCTCGG
GAATTTGTGGCAGATCCGCGAGGTACGGCGGCGTTTCTGACTGCGGAGGAGGATCACCAA
>RXA02562-downstream
TGATGCCGTATATCACCGATATT
>RXA02595-upstream
GTGGGTAAAGGGGACTCCGAGGAAGTCCACGTCGTCTTCTTTCGCGGCGCTGAGGATGGT
TTCGCGGATTTGTGCGGGGGAGTGGGTGGGAGAGAAAACG
>RXA02595
GTGATCGTTGTGGCCATGGCTTCCATTATGGCTTGTTTAAAAGCAGCTAGACTGAATAAC
CCTATGAAGATCCTTTTGTTGTGCTGGCGTGATACCACTCATCCTCAAGGTGGCGGAAGT
GAACGCTATCTGGAGCGGGTGGGTGAGTTTTTGGCGGATCAGGGCCATGAGGTGGTGTTT
CGTACTGCTGGGCACACGGATGCGCCACGGCGTTCTTTCCGCGATGGTGTGAGGTATTCC
AGGAGCGGTGGGAAGTTTAGTGTGTATCCCAAGGCGTGGGTGGCCATGATGTTGGGTCGT
GTGGGGATTGGCACGTTTTCCAAGGTTGATGTGGTGGTGGATACGCAGAATGGCATTCCG
TTTTTTGGAAAGTTTTTCTCCGGTAAGCCGACTGTGTTGCTCACGCATCATTGCCATAAG
GAGCAGTGGCCGGTGGTGGGTCGGGTGCTGGCGAAGGTTGGTTGGCTGATTGAGAGCCAG
ATCGCGCCGCGCGCTTACAAAACTGCGCCGTATGTGACTGTTTCAGAGCCGAGCGCTGAG
GAGCTCATTGCGTTGGGTGTGGATCAGCAGCGGATTCATATCGTGCGCAATGGCGTGGAT
CCCGTGCCGCTGGACACGGCGAAGCTGGATCGCGATGGCCAGCATGCGGTG
>RXA02597-upstream
ATACCCAGTTTGCAAGAATTACAAACGGGGGCAGCCTCAATGACTTGAAAGACTTTATAG
AGTAGAAAGTGAGTCACGACACTTTTTAAAGGAGGATGCT
>RXA02597
TTGCCCGAAGAAGACTTAACGAGCTTGGCCAATGATTGGCTCCAAGCTTTTGAAAAGGCC
ACTGCTAGTTCCAGCCCTGATGAAGCTGCCACTGCAGTCGTGCAACTTTTTGAGGATGAA
GGATAGTGGCGAGACCTTCTTGCATTGACGTGGAACCTCACCACCGCTGAAGGTGGAGAT
GAAATGGCCGAGATGATTCGCAATAGGTGGCCATCAAGCATCTTCCGAAACGTTGAGCTA
AAGGGCGAAGCAGGTGATGAAGGAGATGGTGTCAGTCGCGTACATTTCTCCTGCGAATCC
GCAGACTTGAAGTGCACGGGCATTGTCCGCCTTCGTAATGGCAAGGCGTGGACGCTACTC
ACCTGAGCTCGTGAGCTGCTGGAGCAGCCAGAGCCCAAGGGGCGCAACCGTGAGATGGGC
GTCGTCCATGGACAAAATGAGGACACCCGAAATTGGACTGACCGCAAGAATGATCGACAA
GCAGCGTTGGGTGTCACCGAGGAGCCATACACCCTCATGATCGGTGGTGGACAGGGTGGC
ATTGCCTTGGGCGCACGACTCAAGCGACTTGGTGTACCCGCTCTAATGATTGATAAAGCA
TCTCGCCCGGGCGACCAGTGGCGTAGCCGTTACCATTCTCTGTGCCTGCACGATGCAGTT
TGGTACGACCACGTGCCTTACATTCCATTCGCAGATCATTGGCCAGTATTTACTCCAAAG
GACAAGATGGGTGACTGGCTCGAGCACTATGTCGGCATGATGGATTTGGACTATTGGACC
AACACCGAGTGCCTGCGCGCCTCATACAATGAGGACACCAAGCAGTGGGATGTGACGGTG
AATCGTGATGGCGGGGAGTCCACGCTCCACCCGACCCAACTAGTCATGGGTACTGGAATG
TCGGGCAGCCCGAACAAACCAACTTTGCCTGGGCAGGATAAGTTCCAGGGTGAAATTGGG
CACTCTTCAGAGCACCCCGGCGGCGATGTCGATGGCGATAAGAACGTTGTAGTTCTGGGC
GCTAACAAGTCAGCGCACGACATCTGCGCGGATCTTTATTCCAATGGTGCAAAGCCCGTG
ATGATTCAGCGCTCGTCTACACACATCGTGCGTTCTGATTGGCTGATGCGCGAAGTCTTC
GGGCCTCTCTATTCTGAGGATGCCGTTGAAGCCGGAATTGATACGGATACTGGCGATCTC
CTGTTTGCGTCGTGGCCATATAAGGTGCTGCCAGGTGTGCAGAAGCAGGCTTTGGACAAG
ATCCGTGAGGACGACAAGGAGTTCTACGACAAGCTTGAAAATCCTGGATTCTTGCTTGAT
TTCGGCGATGACGATTCGGGGCTTTTCTTAAAGTACCTTCGCCGTGGCTCTGGCTACTAC
ATCGATGTCGGCGCCTCTGAACTGGTGGCTGATGGAAAGATTCCGGTGCGCTCCAATGTC
AGCATTGAAGACGTCAAGGAAAACTCTGTGGTGCTCACAGATGGTACTGAGCTCCCAGCT
GACGTGATTGTTCTAGCGACCGGCTATGGAAACATGAACAACTGGGTTGCTCAGCTGGTT
GATCAGGAAACCGCTGACAAGGTCGGCCCATGCTGGGGTCTGGGCTCTGAAACCACCAAG
GATCCAGGCCCATGGGAAGGCGAGTTGCGCAATATGTGGAAGCCCACAAACGTGGATTCG
CTGTGGTTCCATGGTGGCAACCTTCACCAGTCACGCCATTACTCACGGTATTTGTCCATG
CAGTTGAAGGCGCGCTACGAAGGTATGAACACTCCGGTGTACAGCAAG
>RXA02597-downstream
TAGATACAAAGAAAAGGGCATCT
>RXA02605-upstream
TGCGATCCTGTCATCTACATGTGGGAGAGTTTCCTTACCCAGGAATTGCCTGCATACCTT
GAGCAGAACTTCGGCGTTGCGCGAAACAACAACTCCATTG
>RXA02605
GTGGCCTGTCCATGGGCGGGAACTGCGGGGCTGAACCTCGCAGCAAAGCACCCAGATCAG
TTCCGCCAGGCTATGTCTTGGTCCGGCTACTTGAACACCACTGCGCCAGGCATGCAAAGC
CTGCTGGGTGTGGCCATGCTGGACACCGGTGGATTCAACGTCAACGCAATGTATGGCTCA
ATCATTAACCCACGTCGTTTTGAAAACGACCCATTCTGGAACATGGGCGGCTTGGCTAAC
ACCGACGTCTACATCTCTGCAGCTTCCGGCCTGTGGAGCCCTCAGGATGATGGAGTTCGC
GTAGACCACCGCCTCACTGGTTCTGTGCTTGAATTCGTGGCAATGACATCCACCAGGATT
TGGGAAGCAAAGGCAAGGCTTCAGGGTCTGAACCCAACTGCGGATTACCCAATGTATGGC
ATTCACGGCTGGGCTCAGTTCAACTCCCAGCTGGAGAGAACTCAGGGTCGTGTTCTAGAC
GTCATGAACGCCTGG
>RXA02605-downstream
TAGAGCCACACCAAAGGCCACAC
>RXA02614-upstream
TCATTGTATACGCCACCCTCGGTCTGCTGTCTGAAGCGCTGATCAGAGCTTGGGAACGTC
ACACCTTCCGCTACCGAAACGCATAAGAAAGTTGCTCGCC
>RXA02614
ATGACTGCCACATTGTCACTCAAACCCGCAGCCAGTGTCCGTGGATTGCGCAAATCATAG
GGAACTAAAGAAGTCCTCCAAGGAATCGACCTCACCATCAACTGCGGCGAAGTAAGCGCG
CTGATCGGACGCTCAGGTTCAGGAAAATCCACCATCCTGCGCGTGTTGGCGGGCCTATCT
AAAGAGCATTCCGGCTCTGTAGAAATTTCCGGAAACCCGGCCGTTGCCTTGCAAGAGCCT
CGGCTGTTGCCGTGGAAAACGGTGCTCGATAATGTGACCTTTGGCCTCAACCGCACTGAT
ATTTCCTGGTCAGAAGCACAAGAACGCGCCTCGGCACTGCTTGCAGAAGTCAAACTTCGC
GACTCCGACGCCGCCTGGCCCCTCAGGCTCTCCGGCGGCCAAGCCCAGCGCGTCTCCCTT
GCGCGAGCGCTCATCTCCGAGCCAGAGCTTTTGGTTCTCGACGAACCCTTCGGCGCCCTC
GATGCTCTGACAAGACTGACAGGGCAAGACCTGCTGCTGAAAACCGTGAACACCCGAAAC
TTGGGAGTTCTGCTGGTCACCCATGATGTTTCCGAGGCCATCGGCCTGGCCGACCACGTC
CTTCTTCTTGAGGACGGCGCCATCACACACAGTTTGACTGTAGATATCCCCGGCGATCGC
CGCACCCACCCCTCCTTTGCGTGCTACACCGCTCAACTCCTTGAGTGGCTCGAAATCACC
ACACCTGCC
>RXA02614-downstream
TAGAAAGAAATCATGAAATTTAA
>RXA02616-upstream
AGTGTTACTTCCGGTAGATAACAAGATGGTCTCAATTAAACTTCGATAGCGTGATATAAG
CTTCGAAAAGTTTTGTGGGAAGAATCGGAAGCAGGCGAAA
>RXA02616
TTGCAGAAGGACACTCGAGGTGGCAAGCACCGCAAGCAGACTACCTCCCCAGTAACTAAG
GGTGGTGTCGCTTTTGTTGCAGTAGCTACCGGTGCCGTGTCAACTGCAGGCGCAGGCGGA
GCAGTTGCTGCACAGGCTTCCAATCAGCCCGTTGAGGTCAACTTCGAGCTTACTGCAAAC
GACACAACTGACCTCGTGGCTGGAAGCTCCGCCCCTCAGATCCTGTCCATCGCTGAGTTC
AAGCCAGTTGTGAACTTGGGCGATCAGATCGTTAAGACCATTCAGTACAACGCTGACCGC
ATTCAGGCTGACCTGGACGCTCGTGGCCCTTCAGTGGTTGGCCCTGCTGAAGGTTCTTAC
ACCTCCGGCTTCGGTGCTCGTTGGGGCACCAACCACAACGGTGTGGATATCGCTAACGCA
ATCGGCACTCCAATCCTCGCTGCCATGGACGGCACTCTTATCGATGCAGGTCCTGCTTCC
GGTTTCGGTAACTGGGTTCGCCTCCAGCACGAAGATGGCACCATCACCGTGTACGGCCAC
ATGGAAACCGTTGAGGTGACCGTTGGTCAGACTGTTAAGGCTGGCGAGCGCATCGCAGGC
ATGGGTAGCCGAGGATTCTCCACCGGCTCCCACCTCCACTTCGAGGTTTACCCTGCAGGC
GGTGGCGCTGTTGATCCAGCTCCTTGGCTTGCAGAGCGCGGCATTACTCTT
>RXA02616-downstream
TAATTAACTTTTGGGCGACCCTT
>RXA02627
GATGTCACTGTGGAAAGCCAACCAGAACGCGTCGTTGCCCTGGGTTGGGGAGATGCTGAG
GCTGCGCTGGAATTCGGTGTGCAGCCTGTGGGTGCATCAGATTGGCTCGCATTCGGTGGT
GAAGGCGTGGGACCGTGGATTGAGGATTGTGCCTACGATGAAGCGCCAGAAATAATCGGA
ACCATGGAACCGGAGTATGAAAAGATTGCAGCGCTTGAACCGGATCTGATTTTGGACGTG
CGCAGCTCTGGCGACCAGGAACGCTATGACAAGTTGTCTTCAATCGCACTGACCATCGGC
GTTCCAGAAGGTGGCGATAGCTACCTCACCCCACGCGCTGAGCAGGTAACCATGATCGCC
ACTGCTCTGGGGCAGGCTGAACGTGGTGAAGAAGTGAACGCTGAATACGAGCAGCTCACT
GCTGATATTCGTGCAGCTCAGCCGGGCTGGCCTGAGAAGACCGCGGCTGGTGTATCTGCA
ACGGCAACCAGCTGGGGTGCATACATCAAGGGCTCCAACCGTGTAGATACTTTGCTGGAC
CTGGGCTTCCAGGAAAACCCTGAGCTGGCTAAACAGCAACCTGGCGATACGGGTTTCTCC
ATCAAATTCAGTGAAGAGACTTTCGGCGTTGTGGATTCCGACCTGGTTGTCGGCTTTGCC
ATCGGTATGACTCCTGAGGAAATGGCAGAGCAGGTTCCATGGCAGATGTTGACCGCCACT
CGTGACGGCCGTTCCTTTGTGATGCCCCGTGAGATTTCCAATGCGTTTTCTTTGGGTTCC
CCGCAGTCCACTCGGTTCGCGTTAGACGCCTTGGTGCCACTTCTGGAGGAGCATGCAGGG
GAG
>RXA02627-downstream
TAGTGGTCCGGTGGTGCGGGCAG
>RXA02628
ATGCTTGAAGGTTTTAGAGATTTCGTCCTTCGGGGAAATGTCATTGAACTCGCAGTTGCC
GTGGTCATCGGTACTGCCTTCACCGCTATCGTGACAGCATTCTCCGAGAGCATCATCAAC
CCATTGATCGCTTCCATGGGCAGCACAGAGGTTGAAGGCCTCGGCTTCGACATCCGCGCC
GGCAATGCCGGAACATTCGTGGATTTTGGTGGTGTCATCACCGCAGCGATCAACTTCCTC
ATCATCGCAGCAATTGTCTACTTCGTTCTCGTTGCTCCAATGAACAAGCTCAGCGAAAGG
CTCGCAAAGCGCAAGGGTGTTGAAGAAGACGAGACCCCAGCTTCCATCGAAGCAGAACTC
CTCACCGAGATCCGCGATCTCGTGGAGGAGCAAAAGCGCCTTCAG
>RXA02628-downstream
TAGTTAAAAGGCCCTAAAAGCAC
>RXA02650-upstream
GAATTTTTGCTGCAACTGTGTAAAAACCAGCGGTGAATTAAAGATCACCTTTCACCCTTA
ATTGAGCCTGGGTGGAAGTTTCTACCGCTCATGGGGAAAG
>RXA02650
ATGGTCAACGTGACCTCAAAGGATGCAGGGGCAAACGTGACCCCCATGAGTAAGAAAGAA
AAGAGGACAACCGTTAAACAGGTGGTTGCCTTGATGGCCGCCATCGTTGTGGTGATTGCG
TCCCTAGACCAAATAGTCAAGGAGATTATGCTTAGTTGGTTGGAACCTGGCGTTCCCGTT
CCCATCATTGGGGATTGGTTCCGCTTCTACCTCCTGTTTAAGCGCGGAGCCGCATTTTGG
ATGGGTGGGGAAAACAGCACCTGGATCTTTACAACCATCCAGTTGAGCTTGGTCATCGGT
ATCGCAATTTATGCCCCACGCATCAAACAGAAGTGGATCGCGGCAGGACTTGCCCTTGTT
GCCGGTGGAGCCTTGGGAAACGTGTTGGACCGGTTGTTCAGAGATCCTTCCTTCTTCTTC
GGACATGTTGTTGATTACATCTCCGTAGGAAACTTTGCAGTATTTAATATCGCCGATGCC
TCGATTTCTTGCGGCGTCGTGGTGTTCCTGATCGGAATGTTCCTTGAGGACCGTGAAAAC
GCCCAGCATGCCAAAGCAACTGACGAGAAGGATGAGGCC
>RXA02650-downstream
TGATGAACAACCGACAAAGCAGA
>RXA02660-upstream
CATTTTTCAGCACACTTTTTAAGATCTCATCGAAAGCGCGATACCCACTATGTCCAATCT
TTTGAGACTTGTCGGCCGACGGCTCATCGCTTTACCGATC
>RXA02660
ATGATTATTGGCGTCACCCTGCTGGTTTTCATCGTCATGTCATTCTCTCCTGCCGACCCG
GCACGAGTTGCCCTAGGCGAATCAGCCTCCCCCGAAGCACTTGAAGCCTACCGTGAAGCC
AACGGCCTCAACGATCCAATGATGGTTCGCTATTTCGACTTCATCCTCGGCATGCTCAAA
GGCGACTTGGGAACCTCTAGCGGTGGCGTAGCTGTTACCGACATTGTTGCCCGCGCTTTC
CCCATCACCCTGCAGCTAACATTCTGGGGACTCATGATCGCTGTTGTAGTGGCGTTGATC
CTCGGTGTCATGGCCGCTCTATACCGAGACCGCTGGCCTGACCAGTTGATTCGCGTGGTC
TGCATTGCGGCTCTTGGTACTCCTTCATTCTGGTTGGCTATCTTGCTGATCGAGTGGTTG
GGTACTATCCCTGGAGCCTGGGGTTTCTTCCGAGCACTTGTCACCCGGTGGGTGCCATTC
AGCGAAGATCCCGCCACCTACTTCAACAACATCGCACTTCAGCGATTGCGTTGGCAGTCC
CCGTTGCAGGTTCTTTGGCCCGCGTTGTTCGTACCTCCATGGTGGAAGAACTGGACAAGG
ACTAGGTCCGCACAGCAATCGGTGCAGGATCCCCAAAAC
>RXA02660-downstream
TGAAGTTGTTGCCCGCAATGTTC
>RXA02661-upstream
TGGACAAGGACTACGTCCGCACAGCAATCGGTGCAGGATCCCCAAAACTGAAGTTGTTGC
CCGCAATGTTCTGCGCAATGCGCTGATCACCCCAATCACC
>RXA02661
GTGATTGGTCTTCGCGTTGGTTCGCTCATGGGTGGTGCGGTGATCATTGAGATCATCTTC
AACATCCAAGCAATGGGACAGCTCATCCTAGACGGTGTGACCCGAAATGACGTCTACCTC
GTCCAAGGTGTCACCCTCACCGTTGCCATCGCCTTCATCATCGTCAATATCGCCGTGGAC
GTGCTCTACGTCCTGGTCAATCCACGTATTAGGAGCATC
>RXA02661-downstream
TAGATGCGCCGTAAACTAACCAC
>RXA02663-upstream
GTGCAAATATCTGTCCAGTTCGTGAGACTACGTCAATGCTTCCAAGGTCATTGGCGCATC
AACCGCTCACATCTTGATCAAGCACGTTGCGCGAAACTGC
>RXA02663
ATGGCTCCGATTCTGGTGTTCGCCACCGTCCTGGTCGCCGATGCGATTGTCTTCGAAGCA
TCCCTGTCCTTCATCAACGCTGGTGTGAAACCACCATCACCTTCATGGGGCAACATCCTT
GCCGATGGTAAAGCCCTGCTGGTTAGCGGCGGATGGTGGGCAACCTTCTTCCGAGGTTTG
ATGATCCTGCTGACCGTTCTCTGCTTGAACATGCTTTCTGAAGGCCTCACCGACACCCTG
GCCAGCCCTAAGCCAAAGCCTGTTTCAGCTTCTGCAAAGAAGGCACTGAAGAAGGAAGAA
TCCGGTGAAAAGGAAGGCTCCGGAATCGTGCTTGGGCACACCACACGTGAAGAAGCCAAC
GCCTCACTGCTCGCATCACTTGCTGCGCTATCCACCAGCGAAAACAATTCCAATAACCGG
CTTATATTTGATGGCAACCCCACTCCTCTGTTGGAAGTTCGCGATCTAAAGATCTCCTTG
GCCAATGCTCACGGAGATATCAATATTGTCGACGGCGTGAACTTCACCGTCGCCCCAGGC
CAAACCATGGGTCTTGTCGGTGAATCCGGCTGTGGTAAATCGATTACCGCAATGTCGATC
ATGGGTCTGCTGCCTCCAACAGCAAAGATCGAAGGCGAGATCCTTTTCGACGGAAAGAAC
CTCCTTGATCTGAAACCAGACGAGCTCAATGCACTGCGTGGACATGAAATCGGCATGATC
TACCAAGATGCACTCTCCTCACTCAACGCATCCATGGTGATCAGCGCCCAAATGAAGCAG
CTGACCCGCCGCGGTGGAAAGCGCAGTGCCGAAGAACTCGTGGAACTTGTAGGCCTTGAT
CCAAAGGGGACCCTGCAGTCGTACCCGCATGAGCTTTCAGGTGGCCAGCGCCAGCGAGTT
CTCATCGCAATGGCACTGACGAGAAACCGACGCCTCCTCATCGCCGACGAGCCAACCACC
GCGCTAGACGTCACTGTTCAGCAGCAGGTTGTGGATCTGCTTAATGAACTGCGTGAAAAG
CTCGGATTCGCCATGATCTTTGTATGCCACGACTTGGCTCTTGTCGCCCGCCTGGTGCAC
AAGCTCACCGTCATGTACGCAGGTCAGGTTGTTGAGCAAGGAACCACCCGCGAAATCCTT
ATCGATCCTCGACACGAATACACCCGCGGTTTGCTCGGATCCGTGCTCTCCATCGAAGCT
GGTGTGGACCGCCTCTACCAGGTCCCAGGCACTGTTCCATCACCAAAGGAATTCGTGGCA
GGCGACCGCTTTGCACCACGATCAGAATTCCCAGAACTTGGCCTTGACCAAAAGCCAGTA
CTTCGCCCCATCACGGGCACAGAGCATGCATACGCAGCAACCGATGAACTTCTTGCCGCA
AAGGGAGAACAACGA
>RXA02663-downstream
TGACCTCGACAATCGACACCAGG
>RXA02664-upstream
ATCCACAAAGAGCCGTACGGGAAAGCTGTTTCGCCCAGACCAGGTCCACGCCAACAAAAA
CATCAACTTCAAGGCCTACCGCGATGAAGTAATCGGCATC
>RXA02664
GTGGGTGAATCTGGTTGCGGAAAATCTACCCTTGCCCGCGTTATGGTTGGCCTGCAACCG
GTCACCTCCGGCGAAGTGCTGTTGAAAGGCAAGCCCATGAAGCCTCGTGGTGCGCAGCGC
AAAGAACTCGGCAGCTCAGTATCCGTCGTGTTCCAGGATCCTGCGAGCTCGTTAAAGCCA
CGAATGACCGTGCGCGAACAGCTCCTGGATCCACTTCGAGTACACAAAGTTGGCGATGAA
GCATCCCGCAACCAGTGGGTTTCAGAGCTGATCTCCATGGTTGGCCTCCCGCAATCCGCG
TTGGAAGTACTCCCCCGACAGGTTTCCGGTGGCCAACGCCAACGCGTGGCCATTGCTCGA
GCACTTGCGCTGAAACCTGACATCATCGTTGCCGACGAACCAACCTCCGCGCTGGATGTA
TGCGTTGGTGCGCAGGTCCTCAACCTTCTGCTGGATCTGAAAACTGAACTCGGCCTGGGA
TTGGTATTCATCTCCCACGAGATCAACACTGTTCGCTACGTTTCTGATCGCATCGCAGTC
ATGCTGGCTGGAGAAATCATTGAGGAAAACACCACCTCAGAGATCTTCAACAATGCGCAG
CAGGACTACACCCGCACTCTGCTCGAAGCGACACCATCGCTGCTGAACAAAACTCGTTTG
>RXA02664-downstream
TAGTCTCCAACCCTTTATTCCCT
>RXA02684-upstream
GCAGGTTCGTCCACAGCCGCCGGTGATTGCAGGGGATGGGGGGAGGCGTCGAAAAGCAAT
ATCTTTTAAGGCCCGTGGCTGCCTGGGCACGATCGCGGGC
>RXA02684
GTGCTTGGTGTGGGCTTGGTGCTTGTGTTTGTGGTGACGCTGTGGGGGGATTCGAAGCTG
AATCGCGTGGATGCCACGCCTGCGACGCAGGTGGCGAACACTGCCGGAACGAACTGGCTG
CTGGTAGGTTCGGATTCGCGGCAGGGTTTAAGTGATGAGGATATTGAGCGGCTAGGTACC
GGCGGCGATATCGGTGTGGGCCGTACGGACACGATCATGGTGTTGCATATGGCGCGTACT
GGCGAGCCGACGCTGTTGTGGATTCCGCGTGATTCTTATGTCAATGTCCCTGGCTGGGGC
ATGGATAAGGCAAACGCCGCATTTACCGTGGGTGGCCCGGAACTGCTGACGCAAACCGTG
GAGGAGGCAACTGGCCTGCGAATTGATCACTATGCAGAAATCGGCATGGGTGGTTTGGCG
AACATGGTTGATGCCGTGGGCGGGGTGGAAATGTGTCCTGCTGAGCCGATGTATGATCCG
CTGGCGAACCTGGATATTCAGGCTGGTTGCCAGGAATTTGATGGGGCAGCCGCGCTGGGT
TATGTGCGCACTGGTGCCACAGCCCTGGGTGATCTGGACCGGGTGGTGCGTCAGCGGGAA
TTCTTGTCCGCTCTGCTGAGTACAGCTACGTCCCCGGGCACGTTGCTGAATCCGTTCCGC
ACCTTCCCGATGATCTCCAACGCGGTGGGAACATTCACCGTCGGCGAGGGCGATCACGTG
TGGCACCTGGCCGGATTGGCGCTGGCGATGCGGGGAGGAATCGTGACGGAGACCGTGCCG
ATTGCCTCATTCGCAGATTACGATGTGGGAAATGTTGCGATTTGGGACGAAGCTGGAGCC
GAAGCACTATTTAGCTCCATGCGC
>RXA02684-downstream
TAAAACCCCAGGTAATCGTTCAC
>RXA02728-upstream
ATCCCATACGTTGCCTTCCGATCGGCATTTTCACAGCGCTGGTCGGCGGCCCAACATTCT
TCATAATGTTGGGCCGAATGATGAAAAGGGCGTGCACTAA
>RXA02728
ATGGCCATTGTTTCCCTCGACAACGTCACCGTATCCATTGAAGGAAAAAAGCTTCTCGAC
GCCGTCTCCCTCAAGGCCTACCCGGGGGAAGTGTTGGGACTCATCGGCCCAAACGGTGCC
GGAAAATCCACTCTGCTGAGTGTCCTTTCAGGCGATCGGCTTCCCGATTCAGGCGAAGTC
AACGTCGGTGGCTTAGATCCCGCAACAGCAGCGGCATCCGATATGGCCAGGGTGCGAGCA
GTCATGCTTCAAGATGTCAGCGTGGCATTTTCCTTCCTCGTGTGGGACGTCGTAGAAATG
GGCAGGCGGCCGTGGCAGAAGGCGTCAACCCCCGAAGAGGATCATGAAATCATCGAAGCA
GCGCTTGCCGCCACCTCGGTATCGCACCTTGCCGAACGTGAAATCACCACACTGTCAGGC
GGCGAGCGGGCACGCGTTGCCTTGTCCCGTGTCCTTGCTCAGCAAACCCCCATTGTGCTG
TTGGACGAACCAACAGCCGCGATGGATATCAGCCACCAAGAACAAACTCTGGGCACAGCG
CGAGCACTGGCAGCCGCCGGGGCAGCAGTGATTGTGGTCCTTCATGATCTCAATGCGGCC
GCTGCATATTGCGACAGCATTGTGTGTCTCAGTGATGGTCGAGTGATTGCCTCCGGTTCT
GTTGATCAGGTGTATTCCACGGAAACGCTGTCCCGTGTTTACGGTTGGCCTATCAGGGTC
GATCATAGTGGAAAATATGTTCGAGTGGAGCCGGACCGTTCTGAGGCGAATTTACCCTCC
GTACTACAGGTGAAAAATACGGTTTCACCAGCT
>RXA02728-downstream
TAGATACATGACTAACTAAGGTT
>RXA02750-upstream
GTTCCATGGACGATGTATTTCTAGCAGTTACAGCTGAACGGAAACGATCATGATTACAGT
TCTGACACGCAGACACTTGCGCTGCTTTTTCCGCGACCGC
>RXA02750
ATGGCAGTGCTGTTTTCCATCATGGGTGCGCTCATCCTTTTGGTCCTGTACGTGCTGTTT
TTAGGAAAACTGCAAATTGACGGTCTCATGGTGGATCTACCTGACTGAGCCCGAGACGAT
GTTGAAGGATTCGTCTTCAATTGGGTGTTTTCCGGAATTCTCATCACGTCCGCAATCACT
GTTCCGCAAGCAGCACTTGGAGTGCTGGTTGAAGATCGCACCCGCGGAGGCATCAAAGAT
TTCCTCGTGGCACCCGTATCCAGAACGACGCTGACGGTGTCCTATATCTTCGCAGCAGTC
ATTGTCGCCATGACGATTTTGATCTTTGAAATCGTGGTGGGAAGTATTGGTTTAGCTATT
TTGGGGCACTTCAGCATGAGCATTGCTCGCGTGCTCGAATTGGTAGTCGCCTTGCTTCTG
CTCACCCTGGTGTTTTCCGCAATTGCAGCATTTCTGATCACCTTGGTGAAATCTCAAGGC
GGAATGTCTGCGCTTTCAAGCCTGGTAGGCACCCTGGCGGGCTTTTTATCTGCTGCTTAT
ATTCCACCCATCGCATTGCCTGAAGCAGTGACAAACGTGTTGAACTTCCTCCCGTTTACC
CCAGCTGGAATGTTGATCAGACAAATTGTGGTTGCCCCAGCATTGGACGCGATTTCACTT
CCACCCGAAGCCTTCGATATCTTCCAATTCGGATACGGACTCAAACTGGAAATGTTTGGG
GAACCCGTTTCTACATGGGTGGCAGTAGGAATTGTTGCCTCATGGGGAGTGGTGTTTGGA
CTCATTGCCGCGTTCAAAATGAAAAGCGTGGTGCGA
>RXA02750-downstream
TAAATCCTGCTAAAGAATGCTTC
>RXA02761-upstream
CAGGTTGTTCTCATTGAGGTGGTTTCTCCGAGAATGCAGCTTTGATCGCCAACGTGGCGC
CAGAAGTGATCGCAGTTGTCGGGCATTCATCGCACTGTGG
>RXA02761
ATGATGGATGGTATCAACCGCCGTACCACCCTCATTACCGGTTATTCTCTCACCACCATT
AGCCACGTATTGATCGGTATCGCATCCGTAGCATTCCCAGTCGGCGATCCTCTTCGCCCC
TACGTTATCTTGACTCTGGTTGTGGTCTTCGTGGGATCCATGCAGACCTTCCTCAACGGT
AGCTACCTGGGTTATGCTCTC
>RXA02761-downstream
TGAGCTGTTCCCGCTGGCAATGC
>RXA02762-upstream
TTCCCAGTCGGCGATCCTCTTCGCCCCTACGTTATCTTGACTCTGGTTGTGGTCTTCGTG
GGATCCATGCAGACCTTCCTCAACGGTAGCTACCTGGGTT
>RXA02762
ATGCTCTCTGAGCTCTTCGCGCTGGCAATGCGGGGTTTCGCAATCGGTATCTCAGTGTTC
TTCCTCTGGATCGCAAACGCGTTCCTGGGATTGTTCTTCCCAACCATCATGGAAGCAGTA
GGACTAACCGGAACCTTCTTCATGTTCGCCGGAATCGGTGTGGTTGCCTTGATCTTCATC
TACACCCAGGTTCCTGAAACTCGTGGACGTACCTTGGAGGAGATTGATGAGGATGTTACT
TCCGGTGTCATTTTCAACAAGGAGATCCGAAAAGGAAAGGTGCAC
>RXA02762-downstream
TAAAAACCCAGACACTGCATAGA
>RXA02769
ACAGTAGTTCCGGTGTACCTCGCTGAACTCGCACCACTAGAAATCCGCGGCTCCCTGACC
GGCGGAAACGAGCTTGCTATCGTCACCGGCCAGCTGCTTGCCTTCGTGATCAACGCGCTT
ATCGCCGTCACCCTACACGGAGTTATTGATGGAATCTGGCGCATCATGTTCGCCGTCTGT
GCCCTCCCTGCCGTCGCCCTCTTCCTCGGCATGCTGCGGATGCCGGAATCACCACGCTGG
CTGGTCAACCAGGGGCGTTACGACGACGCCCGCCGCGTCATGGAGACCGTCCGTACCCCT
GAGCGTGCGAAAGCCGAAATGGATGAAATCATCGCGGTGCACTCTGAAAACAATGCGGCA
GTTCCTGGTGTTAAGCAGTGTTCGGGCCAGGGTTCAGGCCAGGTTTCTAGCAAGCACACC
CACATGTCCATCGGCGAAGTCCTCAGCAACAAATGGCTGGTTCGTCTGCTCATCGCCGGC
ATCGGTGTTGCAGTTGCCCAGCAGCTCACCGGCATCAACGCCATCATGTACTACGGAACC
CGCGTCCTCGAGGAATCCGGCATGAGCGCAGAAATGGCTGTGGTTGCCAACATTGCTTTC
GGTGCCGTTGCCGTCATCGGTGGACTGATCGCACTGCGCAACATGGACCGCCTGGATCGC
GGCACCACCTTCATCATCGGCGTGTCACTGACCACCACCTTCCACCTTTTG
>RXA02795
ATCGACGTCTCCCTCCCCGAACGCACCGCTTCGGCCTACCCACACGAACTTTCAGGCGGG
CAACGCCAACGCGCACTAATCGCAATGGCGCTGGCCAATGATCCTGACCTGTTGATCTGC
GATGAACCCACCACGGCTTTGGATGTGGTTGTGCAAAAACAAATCGTCGATCTGCTGCTG
GGTCTCACCAAAGAACGTGGCACCGCTTTATTGTTCATCACCCACGATCTTGGACTCATC
GCGCGCACCTGCGAACGCTTATTGGTGATGAAATCCGGCGAAACCGTAGAACGCGGCGAC
AGCGAGGCAATTCTTCGCTCCCCCGCCCATTCGTATACCCAACAGCTCCTTGATGCTTCA
ATCCTTGACCAGCCAGAAATCGCCTCAGATTCTGGCGCGCCGGTAGTGATTGATGTGGAG
GAGGCGTCGAAAAGCTTTAAAGAAACCACCGCCCTCCACAAGGTTTCATTGGCGGTGCGC
AAAGGTGACCTGCTTGGAATAGTCGGCGGATCAGGTTCCGGCAAAACGACTCTGCTGAAG
CTCATCGCCGGTTTGGATAAGCCCACAACCGGTACCGTTGCGGTAACCGGTGGTGTGCAG
ATGGTGTTTCAGGATCCCCAATCAAGCCTCAACCCACGGATGAAAATCAAAGACATTGTC
GCCGAACCACTGCTTGGTTGGAACGCGGCGGAGAAAACCACACGGGTTGCGGAAGTCATC
ACCCAAGTGGGACTGAGCCCCGATGTCTTAGATCGCTACCCCCACGAATTCTCCGGAGGA
CAGCGCCAACGAATCTCCATCGCGAGAGCCCTGGCCATCAAACCAGCGATCGTGCTTGCC
GACGAACCTGTCTCCGCCCTCGATGTGTCCGTACGTAAACAAGTACTGGATCTTCTCCAA
CAACTCGTCGAAGAATACGGCATCACCTTGGTGTTCGTCTCCCACGATCTGGCAGTGGTG
AGACACCTGTGCACAACGGTGTGGGTGATGGAACAGGGACGAGTCCTTGAGCAAGGGCCC
ATCGATTCGGTTTATGATCACCCAGAGAGCGAATACACCAAGGAGCTGCTTGATGCCGTT
CCGCGGTTGAGCCTT
>RXA02795-downstream
TAAACCAGCGCAGATGACAACGC
>RXA02808
TTTTACTTCGGCATCCTCCGAGTCCTTGCAGAAAGTGCTTCGCACTTCGGCATCGAGCCT
GTGGAAATGGCCCGCGCATCCATCACTGGGCAGCCCGTTCACATGCAAAGGCGGCTGGTC
CCAGCGATCGTCCTGCTGGTTTCCCTCGCCAACGTCAACCTTGGCGACCAGGACAAGAAG
GTTCTGTGGCGCGCCTGCATCGTGTCCATCGCGATGCTCGCCGTAGCCCTCTTCATCGGC
GTCGTGCCACTCAGCGCA
>RXA02808-downstream
TAAAATAGCTTTTCGACGCCAAA
>RXA02863-upstream
AGGGACTGCCCATTGCGGTGCGCGATGATCCCGAAACCAGCTCACTTCGCGTGATCCCGC
ATCCAAATCCCTTTTGATTGAAAGTTTGACTTAAAAACCC
>RXA02863
ATGAAAAAATCACTCATCGCCATTGTTGCCAGTGCGCTCGTGTTAAGCGGCTGCACCTCT
GATTCTTCTGACTCTTCCGGCACTTCCGGAACTGTGGAAACCACTTCGATTACAACCAGC
GTTGCCGCAGCTGACGGCGCATTCCCACGCACCGTCACACTCGACGATTCCTCCATCACC
TTAGAATCCAAACCAGAGCGCATCGCCGTACTCACCCCAGAGGCAGCATCCTTGGTTCTC
CCCATCACAGGCGCCGACCGCGTCGTGATGACCGCCGAAATGGACACCGCTGACGAAGAA
ACCGCAGCTCTGGCCTCCCAAGTGGAATACCAAGTCAAAAACGGTGGCAGCCTCGACCCC
GAACAAGTTGTCGCCGGCGACCCAGATTTGGTGATCGTCAGTGCGCGTTTCGATACCGAA
CAAGGCACCATCGACATTTTGGAAGGCCTCAACGTCCCCGTAGTTAACTTCGATTCAGAC
GCTTGGGGAGACATCGACGCCATCACCAAACACCTAGAAATTGTGGGTGAACTCGTCGGC
GAAGAAGACAAAGCCGCAGAAGCAATCGCAGAAATCGATGCAAACCGCATCGACATCGAC
AAGCCTGCCACCTCCCCCACTGTGCTCACTTTGATGCAACGCGGACCACGCCAAATGGTC
ATGCCAGAATCTGCCATGCTCAACGGCCTGATCCGCGAAGCCGGCGGCACTCCAGTGGTA
GATTCTCTCGGCGCGGTAGGCACCATCACTGCAGACCCAGAACAAGTTGTTGCGATGGCA
CCTGAGATCATCATCATTCAGGACTTCCAAGGTAAAGGCCGAGAGAACTTCGCTAATTTC
CTCTCCAACCCAGCGCTAGCCAACGTTCCCGCCATTGAAAACGACAAGATTTTCTACGCC
GACACTGTCACCACTGGAGTTACTGCAGGTACCGATATCACCACTGGTCTGCAGCAAGTG
GCAGAAATGCTGAGC
>RXA02863-downstream
TAGTTTTGAGATGTTGAAACTAG
>RXA02864-upstream
CTTGCAAACAGGCGTGGTGGTGGCGTTCATTGGCTCACCAATTTTCCTTTATTTACTGCT
CAGCATGCGCAAGCGACGCGGATTGGGGCTGTAAAAACTC
>RXA02864
ATGCCTCAATTAGTTGAAATTGGTGATCTCAACGTTGAATTCCCCTCTCGCCATGCAGTG
AAAAACGTGTCTTTTTCTGCACCTGCTGGAAAAGTCACCGCACTGATTGGCCCAAATGGT
GCTGGTAAAAGTACTGCCCTTTCGGCGATTGCAGGATTGGTTGAATCCACCGGCGAGGTA
ATGGTTGGTGGGAGTGGGGTTGCGTCGAAAAGCGCTAAAGCCCGAGCCCGCCTGCTCTCA
CTCGTGCCGCAAAACACCGAGTTGCGCATTGGTTTTAGTGCACGCGACGTTGTCGCGATG
GGCCGCTACCCGCATCGTGGCCGCTTCGCCGTGGAGACCGACGCAGATCGACGCGCCACC
GATGACGCCCTGCGCGCCATCAACGCGCTCGACATCGCCGAGCAGCCCGTCAACGAATTA
TCGGGCGGCCAGCAGCAGCTCATCCACATCGGCCGAGCGCTCGCCCAAGACACCGCCGTC
GTGCTTCTCGACGAGCCCGTCTCCGCCCTTGATCTACGGCACCAAGTTGAAGTCCTTCAA
CTCCTGCGCGCCCGAGCTAATTCCGGCACCACCGTGATCGTCGTCCTTCACGATCTCAAC
CACGTTGCCCGTTGGTGCGACCATGCAGTGTTGATGGCCGACGGCGAAGTTGTCTCCCAA
GGTGACATCCGCGAGGTGCTCGAACCTGCGACACTGTCCACCGTGTACGGACTGCCCATT
GCGGTGCGCGATGATCCCGAAACCAGCTCACTTCGCGTGATCCCGCATCCAAATCCCTTT
>RXA02864-downstream
TGATTGAAAGTTTGACTTAAAAA
>RXN00001-upstream
TGTCATAGGCAGCACTCTAGATGGCGCACAGTGACTCACTTCACTGTTTCTCACACTACG
GATCGTTGGGCACGTACCTGCCGATGGAGGAGATTCTGCA
>RXN00001
ATGGCAACCGTAACGTTCAAAGATGCTTCCCTAAGCTAGCCGGGAGCAAAGGAACCCACC
GTCAAGAAATTCAACCTGGAAATCGCCGATGGCGAGTTCCTCGTCCTGGTCGGCCCTTCC
GGCTGTGGTAAATCCACCACGCTGCGCATGCTGGCCGGTTTGGAAAACGTTACTGACGGT
GCCATTTTCATCGGAGACAAGGACGTTACCCACGTTGCACCGCGTGACCGTGACATCGCC
ATGGTTTTCCAGAACTATGCTCTCTACCCCCACATGACCGTGGGCGAGAACATGGGCTTC
GCACTGAAGATCGCCGGCAAGTCCCAAGACGAGATCAATAAGCGCGTCGACGAAGCCGCC
GCCACTTTGGGCCTGACCGAATTCTTGGAGCGCAAGCCGAAGGCCCTGTCCGGTGGTCAG
CGTCAGCGTGTGGCCATGGGCCGCGCCATTGTTCGCAACCCGCAGGTCTTTCTCATGGAT
GAGCCGCTGTGTAACCTCGATGCCAAGCTGCGTGTTCAGACCCGTACGCAGATTGCAGCC
CTGCAGCGCAAGCTTGGGGTTACCACCGTTTACGTCACCCACGACCAGACGGAGGCCTTG
ACCATGGGTGACCGCATCGCGGTGCTGAAGGATGGCTACCTGCAGCAGGTTGGCGCGCCC
CGAGAGCTTTATGACCGCCCCGCGAACGTCTTCGTCGCGGGCTTCATCGGCTCCCCAGCC
ATGAAGTTGGGCAGCTTCTCGGTCAAGGATGGTGACGCTACCTCTGGTGACGGTCGCATC
AAGCTTTCCCCGGAAACGCTCGCCGCCATGACGCCGGAGGATAATGGCGGCATCACCATT
GGTTTCCGCCCGGAGGCACTGGAGATCATTCCGGAAGGCGAGTCCACCGATGTTTCCATC
CCAATCAAGCTCGACTTCGTGGAGGAACTCGGTTCCGATTCCTTCCTCTACGGCAAGCTG
GTAGGCGAGGGCGACCTTGGATCCTCCAGCGAGGATGTCCCCGAGTCCGGCCAAATCGTC
GTCCGCGCTGCTCCGAACGCCGCGCCTGCTCCGGGCAGTGTTTTCCACGCACGCATCGTG
GAGGGCGGCCAGCACAACTTCTCGGCGTCGACTGGCAAGCGCCTCCCT
>RXN00001-downstream
TAAGCGCGCGTACCGGCTACCCC
>RXN00099-upstream
CTCTGGTGAAGAGGATGTTGACTCGGGAGATTCTTCCACTGATTCACTGATTAAGTGGTA
CCGCGCAAATAGGTAGTCGCTTGCTTATAGGGTCAGGGGC
>RXN00099
GTGAAGAATCCTCGCCTCATAGCACTGGCCGCTATCATCCTGACCTCGTTCAATCTGCGA
ACAGCTATTACTGCTTTAGCTCCGCTGGTTTCTGAGATTCGGGATGATTTAGGGGTTAGT
GCTTCTCTTATTGGTGTGTTGGGCATGATCCCGACTGCTATGTTCGCGGATGCTGCGTTT
GCGCTTCCGTCGTTGAAGAGGAAGTTCACTACTTCCCAACTGTTGATGTTTGCCATGCTG
TTGACTGGTGCCGGTCAGATTATTCGTGTCGCTGGACCTGCTTCGCTGTTGATGGTGGGT
ACTGTGTTCGCGATGTTTGCGATCGGAGTTACCAATGTGTTGCTTCCGATTGCTGTTAGG
GAGTATTTTCCGCGTCACGTGGGTGGAATGTCGACAACTTATCTGGTGTCGTTCCAGATT
GTTGAGGCACTTGCTCCGACGCTTGCCGTGCCGATTTCTCAGTGGGCTACACATGTGGGG
TTGACCGGTTGGAGGGTGTGGGTCGGTTCGTGGGCGCTGCTGGGGTTGGTTGGGGCGATT
TGGTGGATTCCGCTGTTGAGTTTGCAGGGTGCCAGGGTTGTTGGGGCGCCGTCGAAGGTT
TCTCTTCCTGTGTGGAAGTCTTCGGTTGGTGTGGGGCTCGGGTTGATGTTTGGGTTTACT
TCGTTTGCGACGTATATCCTCATGGGTTTTATGCCGCAGATGGTAGGTGATCCTCAGCTC
GGTGCGGTGTTGTTAGGCTGGTGGTCAATTTTGGGATTGCCGCTGAACATTCTGGGACCG
TGGTTGGTGACGCGTTTCACTAACTGCTTCCCGATGGTTGTTATCGCCAGTGTCATGTTT
CTCATCGGTAATGGTGGGTTTTGTTTGGCTCCGGATGTTGCGCCGTGGTTGTGGGCGACG
TTGTCTGGTCTTGGTCCCCTTGCGTTCCCGATGGCGTTGACGCTCATTAATATTCGTGCT
GAAACTAGTGCTGGTGCTTCTGCGTTGAGTTCCTTCGGGCAGGGTTTGGGTTATACGATT
GCGTGTTTCGGTCCCTTGTTGACTGGTTTCATTGTCGATGCGACAGGCAGCTTCCGAACA
ATCTTTGTGCTTTTTGCGGTTGCAACACTCTTCGTTATTAGAGGCGGTTACTTTGCGACA
AGGCAGGTTTACGTCGAAAAGCTTTTAAATCGC
>RXN00099-downstream
TAGGATGGCGCTATGCCGCAAAG
>RXN00193
AAAGCTTTCTNCCAACGCGAAGGTTTCATCTCAGCGTTCGGTTTCACCGTCGTCGTGGTC
ATCGTCTCCGTGATCACAGTCAACATGTTCGCCTTCCTCTTGGCGTGGTTGCTGACCCGC
AAACTCCGCGGTACCAACTTTTTCCGCACAGTCTTCTTTATGCCGAACCTTATCGGCGGC
ATTGTGCTGGGTTATACCTGGCAGACCATGATCAACGCCGTGCTTTCGCACTATGCCACG
ACTATTAGCGCGGACTGGAAATTCGGCTACGCCGGCCTCATCATGCTACTTAACTGGCAG
CTCATCGGCTACATGATGATCATTTACATCGCCGGCCTGCAAAACGTCCCACCAGAGCTC
ATTGAGGCTGCCGAACTCGACGGCGTCAACAAGTGGGAGATGCTGCGGCACGTCACTATT
CCGATGGTCATGCCATCCATCACCATCTGCCTCTTTTTGACTTTGTCGAACTCCTTTAAG
CTCTTCGAGCAGAACCTGGCGCTGACCAACGGCGCTCCTGGGGGGCAAACTGAGATGGTG
GCGCTGAACATCATCAACACGCTGTTTAACCGTATGAATGTCGAGGGCGTCGGT
>RXN00378-upstream
ACCGTGAGCCTTATACTGTGAGGACATTAAAAGTGACACGTCTTTTTCTATCTTTTACAA
CGCAAGAAGGTTTATCGTGAGCACACCGGATTCTTGCTCG
>RXN00378
GTGGACAAGGCCGTAAACACTGCTATCTCTGACGCCAAAACAGCGGCGCTCAAGGCAGGT
GTTGGATTGAACCGAGCCACCGCCTCAGAAGAAGAGGAAGATTTAAGCTCAAGCATTAAG
GTTTCTTTGGCCTTTGAGCTCGAGGGGTTAAGCAATGCACCATCGTTGATGGTGGTGGAA
AAAGCCGTAGAGAAGATCCCCGGTGTATCCGCGGATCTGATTTACCCTTCACAAACTGCA
TGGATTACAGCAACTGATCGGGTACATCCCGAAACCCTCATTGAGGTGTTTGAGCAGTTC
GGCATCAAAGCACACCTTTCTAATTCATCGCTGCTGCGCAGGCATCAACAGCTCAGCGCG
GAAGTAAATAGGGAAGCACGCCTTGATCGTTACCGCTCCCGAATGGATGCCAAGCGAATC
TCGCCTCGTGTGCGAAGGCATAACCGACAAGAAATGGTACATGCGGTACGCGCTCGTGAA
AGTGGTTGGATTAAACGCAGGAATCACACCACCTCGCAGCATGAAGACCCAATGTCGGGC
GATGTGCTGTTCACCGCCCGCGCACTGATTACACCTAAGCGTTTGTGGGTGTCGTTGCCG
TTTGCGCTCATCGTATTGGCGTTATCGTTGAATCCTTCGTGGCAGTTTGATTATTGGCAG
TGGTTGTCCGCTGTGTTGGCTATTCCTGTGGTGGTGTGGGGTGCCTGGCCGTTTCACCGC
GCTGCAGCAGGCGGTATTCGTCGAGGAATTTCCGCTCTTGATGCGACCAGCTCAATCGCT
ATTGCTGCTGCATACGCGTGGTCTATCGCCATGCTGTTGTTTGAAACCCCAGGAGGTAAA
TCCTGGCGGTCATATCCGTCCTGGTTCGCTTTTGACCACGGCACGTTGACCCAAAACGAG
ATTTATTTTGATGTGGCCTGCGGAATCACCGTGTTGCTTCTTGCCGGACGGCTGCTGACA
AGGCGTCGAAGCCAATCCAGTTTGTTAGCGGAACTTGGTCGCCTCCAAATCGATCCACAG
CGCATTGTCACTGTGGTGCGTAAACACCGATTGAAGCGCGTAGTCCAGGAACTGAACATT
CCAGTGCAGGAAGTCCGTGTCAATGACGATGTGAAAGTTCCACCTAATACCACGATCCCT
GTGGATGGCACTGTCATCGGTGGCGGTTCGCGGATCGCAGCTAGCATCATCATGGGACAA
GACCAGCGTGATGTAAAAGTAAATGACAAAGTTTTCGCCGGCAGCCTCAACCTCGAATCC
GAAATCAAGGTTCGTGTTATTCGCACTGGTCACCGCACCCGCATCGCCGCGGTACATAGG
TGGGTTAAAGAAGCGACGTTGAAGGAAAACCGCCACAATAGGGCAGCGATCCGTTCGGCC
GGTAACCTTGTGCCCATCACGTTCACCCTTGCTGTGGTGGACTTGTGTCTGTGGGCACTG
ATCTCTGGAAACATCAACGCTGCATTTAGCACTACCTTGGCTGTCCTTGGGTGCGTGGCT
GCGGTGGCCTTAGCGTTGTCTGCTCCACTTGCCACGAGGAATTCCATGGAAGCTGCAGCA
CGACACGGTATTTTGGTCCGCTCTGGTGAAATTTTGCGAGTTCTCGATGATGTGGATACT
GCCGTATTTAATCGTGTGGGCACACTAACCGATGGCGAAATGACAGTGGAAACCGTCACA
GCAGACAAAGGCGAGGACCCAGAACTAGTGCTGCGTGTCGCCGGGGCGTTGGCCATGGAA
TCCCACCACGCGATTTCCAAAGCACTGGTGAAAGCATCCCGTGAAGCTCGTGATACCGGC
GCCGGTGGTGAAGATGTCCCACACTGGATTGAAGTAGGCAACGTGGAAATCACCGAAGCC
GGCTCATTCCAAGCAACCATCGAGCTGCCACTGATCAAACCATCTGGCGAAAAAATCATG
CGCACCACAGAAGCACTCCTGTGGCGACCACGATCCATGACAGAAGTCCGTGAGCACTTA
AGCCCCCGACTAGTGGCAGCAGCAACCTCAGGTGGCGCACCACTGATCGTGCGATGGAAA
GGCAAAGACCGCGGAGTTATCACTCTAAGTGACCACGTGAGATCAGATTCCTCCGATGCG
ATTATTGCGATTGAAGAACAAGGCATCGAGACCATGATGCTTTCACGTGATACTTACCCG
GTGGCACGTCGATACGCAGACAGCTTAGGCATCACCCACGTCTTGGCCGGCATCGCGCCG
GGCAAGAAAGCCCAGGTCGTCCGTGCAGTCCACACCCGCGGATCCACTGTCGCGATGATC
GGCGATGAATCAGTAATGGACTGTTTGAAAGTCGCTGACGTGGGTGTACTGATGGGCGTC
GATCGTCCCTCAGATCTGCGTGATGATTCCGATGACCCGGCAGCTGACGTTGTGGTCATG
CGCGAAGAGGTCATGAGCGTGCCGACGCTGTTTAAACTGGCTCGACGCTACGCCAAGTTG
GTCAATGGCAATATTGCTCTGGCCTGGATCTATAACGGTGTTGCCATGGTGCTTGCAGTG
TCTGGCTTGCTGCATCCAATGGCTGCGACCGTGGCTATGCTGGCGTCTTCGCTGCTTATT
GAATGGCGCTGGGGCAGGGCGGGCAAGTAC
>RXN00378-downstream
TAACCAGCAATTCCCAAGCCCAA
>RXN00412-upstream
GTTTTGACGAACACCACGTCGCGTACGGTTCCTCGGGGCGTTAAACTATTTGTCTTCCAG
GTTTTGTCCCCCGACTTTTGTACGAATCGAGGACACCGTC
>RXN00412
GTGTCACACACCGCGTCCACACCGACGCCAGAGGAATACTCCGCGCAGCAACCCAGCACC
CAGGGCACTCGCGTTGAGTTCCGCGGCATAACCAAAGTCTTTAGCAACAATAAATCTGCT
AAAACCACCGCGCTTGATAATGTCACTCTCACCGTAGAACCCGGTGAGGTAATCGGCATC
ATCGGTTACTCTGGCGCCGGCAAGTCCACTCTTGTCCGCCTCATCAATGGCCTTGACTCC
CCCACGAGCGGTTCGTTGCTGCTCAACGGCACGGACATCGTCGGAATGCCCGAGTCTAAG
CTGCGTAAACTGCGCAGTAATATCGGCATGATTTTCCAGCAGTTCAACCTGTTCCAGTCG
CGTACTGCGGCTGGAAATGTGGAGTACCCGCTGGAAGTTGCCAAGATGGACAAGGCAGCT
CGTAAAGCTCGCGTGCAAGAAATGCTCGAGTTCGTCGGCCTGGGCGACAAAGGCAAAAAC
TACCCCGAGCAGCTGTCGGGCGGCCAGAAGCAGCGCGTCGGCATTGCCCGTGCACTGGCC
ACCAATCCAACGCTTTTGCTTGCCGACGAAGCCACCTCCGCTTTGGACCCAGAAACCACC
CATGAAGTTCTGGAGCTGCTGCGCAAGGTAAACCGCGAACTGGGCATCACCATCGTTGTG
ATCACCCACGAAATGGAAGTTGTGCGTTCCATCGCAGACAAGGTTGCTGTGATGGAATCC
GGCAAAGTTGTGGAATACGGCAGCGTCTACGAGGTGTTCTCCAATCCACAAACACAGGTT
GCTCAAAAGTTCGTGGCCACCGCGCTGCGTAACACCCCAGACCAAGTGGAATCGGAAGAT
CTGCTTAGCCATGAGGGACGTCTGTTCACCATTGATCTGACTGAAACGTCCGGCTTCTTT
GCAGCAACCGCTCGTGCTGCCGAACAAGGTGCTTTTGTCAACATCGTTCACGGTGGCGTG
ACCACCTTGCAACGCCAATCATTTGGCAAAATGACTGTTCGACTCACCGGCAACACCGCT
GCGATTGAAGAGTTCTATCAAACCTTGACCAAGACCACGACCATCAAGGAGATCACCCGA
>RXN00412-downstream
TGAACGAGATGATCCTCGCAGCT
>RXN00431-upstream
TGGATCGTCCTCGCCTTCACATTCGTCCGCCTTGGCCTTGCTCTCCTCGCGATGAAGCAA
TGGCGATTCCGCGTCAGCTACTGGGTATAAGGAGCACCAC
>RXN00431
ATGGTATCCATCGATACATACAACGCCTGCGTCGACTTCCCCATCTTCGACGCCAAATCC
CGCTCCATGAAGAAAGCCTTCCTCGGCGCAGCCGGCGGAGCAATCGGGCGCAATCAAGAC
AACGTCGTAGTCGTCGAAGCGCTGAAGAACCTCAACCTGCACTTGCGCGAAGGTGACCGG
GTCGGACTCGTCGGCCACAACGGCGCCGGCAAATCCACCCTCCTGCGACTCCTCTCCGGC
ATCTACGAACCCACCCGCGGAAGCGCTGACATCCGTGGACGCGTCGCCCCCGTCTTCGAC
CTCGGCGTCGGCATGGATCCAGAAATCTCCGGCTACGAAAATATCATCATCCGCGGCCTC
TTCCTCGGTGAAACCCGCAAACAGATGAAAGGCAAAATGGAAGAAATCGCCGACTTCACC
GAACTCGGCGAATACCTCTCCATGCCTCTCCGAACCTACTCCACCGGCATGCGCATCCGC
GTAGCCCTCGGCGTGGTCACCTCCATCGAGCCCGAAATTCTGCTTCTTGATGAAGGCATC
GGCGCCGTCGACGCCGCCTTCATGGGCAAAGCCCGCGACCGCCTCCAAGCCCTCGTCGAA
CGATCCGGCATCCTCGTCTTCGCCTCCCACTCCAACGACTTCCTCGCCCAACTCTGCAAC
ACCGCACTCTGGGTCGACCACGGACAAATCCGCGAAGCGGGACTAGTTCCAGACGTGGTG
GAAGCCTACGAAGGCAAGGGCGCCGGCGACCACGTCCGCAGACTCCTCACCCGCATGGAA
GAAGAAAAG
>RXN00431-downstream
TAGCTCCTGCGTTTCGGGTTTGC
>RXN00444-upstream
TACCCAATGGCATTGACCACCACCGGTGAAGACAACGAGGTAGCGAAGGCTTTCGCAGAG
TTCCTCAGCAGCGATCGTGCCAAGGAGATCCTTGCCAGCT
>RXN00444
ATGGTTTTGGCACAAACTAAAAAGGCTCGTCGAAGCGAGAATCATATCCTCCCAGGGTGG
TTGCTCATCCCAGCCACCCTGGCCATGCTGCTGATCATTGGACCTATTTTTGCTTTGCTG
TTGCAGATCCCCTGGGATCGGTCTTGGGAGTTGCTTACCGCGCCGGAATCTTTAGGAACC
GCACGGTTATCTATCGGAACTGCTCTGTTTTCTACCGCGCTATGCGCAATTGTGGGTTTC
CCGCTAGCGTTGGCGCTGCATTTATATGAGCGTTCGCACCCCAGGGTGACATCAGTTTTG
ACGGTGCTGGTTTATGCGCCTTTGGTGTTGTCGCCGGTGGTGTCTGGTTTGGCGCTGACT
TTTCTGTGGGGCAGGCGTGGTTTTTTAGGTTCTTGGCTTGATCAGGTTGGATTGCCGATT
GCATTTACCACCACGGCTGTGGTGTTTGCCCAGGTGTTTGTAGCGTTGCCATTTTTCATT
TCCACTGTGACTACTGCACTGCGTGGCATTCCAAAACAGTTTGAGGAAATCGCAGCTACT
GAAGGCGCAACCCGCTGGGAGATGATGCACAAGATGATCATTCCGCTGGCGATGCCTGGA
ATTTTCACCGGTATGATTTTGGGATTCGCCAGGGCCTTGGGCGAGTATGGTGCGACACTG
ACTTTTGCTGGAAATATTGCAGGTGTTACCCGCACCATTCCGTTGCATATTGAGCTTGGT
TTGAGTTCCAATGACATGGATAAAGCCTTGGGAGCGGTGATTATGCTTTTGGCTGTCTAT
GTCCTCATCATTGGAGCCATCGGAGCGTTACGATTGTTTTCCAAGGTGAGAAAGGTT
>RXN00444-downstream
TAATTGATGTCTCGTTCGCCGGA
>RXN00466-upstream
TTTAAAAGCGCACTAAGAGCTCGTCAATTCTTTAAAACAAGCTGAGAATGTGAATAATAG
GATAGGTTAACCTGATTCGATTAGAAAACGGAGATTTGTC
>RXN00466
GTGCAATCCCGCCTGTCCAAAATCCTGCGCAGTAGCGTCGTAGGCGTTGCTGTCCTAGCC
CTGTTAGCTGGGTGTTCTAACAATGCAGATGACACCGACGCTGATTCAACATCCACGGGA
AACTCCGCTTTTCCTGTTTCGATTGAACACGAGTTCGGAACCACCACAATCGATGATGTA
CCCGAAAGAGTTGTCAGGCTTGGCGTTACCGACGCCGATATTGTCCTGGCATTGGGGACC
GTCCCAGTAGGCAACACCGGATACAAATTCTTCGAAAACGGATTGGGACCGTGGACTGAT
GAGTTAGTGGAAGGCAAAGAATTAACACTGCTTGACTCTGATTCCACACCAGATCTTGAA
CAAGTAGCAGCCCTGGAGCCAGACCTGATTATTGGAGTCTCTGGGGGGTTTGACGACGTT
GTATACGAGCAACTATCTGATATCGCACCGGTGGTCGCCCGTCCAGCGGGAACAGCTGCA
TACGCAGTAGCTCGCGAGGAAGCTACCAACCTTGTTGCCCGTGCGATGGGGCAATCAGAA
AAAGGACAAGAGCTCAATGAGGAAACAGATGCTCTGATCCAAGCTGCGCGTGATGAAAAT
CCTTCTTTTGACGGTAAAACAGGAACCGTCATCTTGCCATACCAGGGTAAATACGGTGCC
TACCTGCCAGGCGATGCACGGGGACAATTCCTCGATTCACTTGGCATTTCGCTGCCGGAA
GCAGTTCTTTCGCGAGACACCGGCGACAGCTTCTTTGTCGATGTCCCCGCTGAAAGCGTC
AAAGACGTAGACGGTGATGTTCTCCTCGTGCTTTCCAACGACGAAAATCTGGATATCACA
GCAGAGAATCCACTGTTTGAAACACTGAACGTTGTGCAAAAAGACGCAGTAATTGTGGCA
ACAACGGAAGAACGCGGGGCGATTACCTACAACTCAGTGCTGTCTGTTCCTTTTGCGTTG
GAACATCTCGCACCACGTATTGCTGAGGCTTTGAAG
>RXN00466-downstream
TAAAACTCAACTACTCGAGCACA
>RXN00523-upstream
TGGTGACTCGTCCGAGTGAAATTGCCGTGGGCATCATCATGCCGATCATTGGTGCGCCAC
TGTTTATTTGGATTATTCGTCGTCAGAAAGTCAAAGAGCT
>RXN00523
ATGAGCCTTAGCCATCAACTCAAGCGCCAGCGCGCATCGCGCAACTCCGGCAGGTGGCTG
ATTGTTGCGGCATTGGGCGTCGTCACGCTTGGTATTTTTGCTTTTTCTTTGATGTGGGGC
GAGGTGTTTTATGGCCCTGCTGAGGTGCTGAAAGTGTTGTCTGGACAGCAGGTTCCCGGC
GCGAGTTATTCCGTTGGCGTGTTGCGTTTGCCGCGCGCGGTGATGGGTTTGACTGCGGGT
TTGGCGTTTGGCGCGGCGGGCGTGATTTTTCAGACGGTGTTGCGTAATGAGTTGGCGTCG
CCGGATATTATCGGCATTTCTTCTGGCGCGTCGGCGGCGGGCGTAATTTGCATTGTGTTT
TTCGGGATGTCGCAGTCTGCAGTGTCGGCGATTTCTTTGTGTGCGTGCTTGGCTGTGGCG
TTGTTGATTTATCTGGTGGCGTATCGCGGTGGTTTTTCGGCCACGCGTCTGATTCTTACC
GGCATTGGTATTGCTGCGATGCTGAATTCATTAGTGTCGTATTCGCTGTCCAAGGCTGAT
TCTTGGGATCTGCCGACCGCGACGCGCTGGCTTACCGGCTCGCTCAATGGTGCGACGTGG
GATCGTGCGATGCCGCTGATTGTCACCACTGTGGTACTCATTCCGCTGCTGGTGGCTAAT
GCGCGCAATGTGGATCTTATGCGTTTGGGCAATGATTCCGCGGTGGGTTTGGGCGTTGCT
ACTAATCGCACGCGCGTCATTGCGATTATTGCCGCTGTTGCGCTCATCGCCGTTGCTACC
GCTGCATGCGGCCCGATCGCATTCGTGGCGTTTGTGTCTGGCCCCATTGCCGCGGGCATT
TTAGGCTCCGGCGGATCGCTCATCATCCCCTCCGCACTCATCGGCGGGTTGATCGTGCTC
ATCGCCGACCTAATTGGCCAATACTTCCTCGGCACCCGCTACCCCGTCGGAGTTGTCACC
GGCGCATTCGGCGCCCCATTCCTTATCTATTTACTCATTCGTTCCAACCGCGCGGGAGTA
ACCCTG
>RXN00523-downstream
TGACCACCAACGATCAACTATCC
>RXN00525-upstream
CCATCGTGTTTATTACTCACAACCCTGAGCTTGCTGATGAATCTGATGGGGTGGTCACCA
TGGTTGACGGGCGCATCATTGGGTCTGAGGTGAAACACTC
>RXN00525
ATGAGCCTTGCAGAATCAATTCTTTTGGCGCTCACCAGCCTGAGAAGCAACAAGATGCGT
GCATTGTTGACGCTGTTAGGAGTCATCATTGGTATCGCATCAGTCATCGGAATTTTGACG
ATTGGTAAAGCCCTGCAGGATCAAACTTTGAATAGTTTGGAAAGGTTGGGCGCGAATGAT
CTGTCGGCGCAGGTGGAGGAACGCCCGGACGAAGATTCCCCGGAACCCGATATGTTCGCT
TTTTCTGGGGCTGCAAACTCTAGTGGCAATCTGATTCCGGAAGAAACAGTTGATACGCTG
CGCGATCGTTTCGCAGGCAGCATGACGGGAATCAGCGTTGGCGGAATGGGTACGCAAGGC
ACTCTCATCGGCGACACCGCAGATCTTAAATGCGATGTCCTCGGCGTCAACGAGGATTAT
ATGTGGATGAATGGCGTCGAAATGAACTACGGCCGCGCCATCACGCAAGACGATGTTGCC
GGTGAGCGCCCCGTTGCGGTCATCGCCCCAGACACCTTTAATACGCTTTTCGACGCAAAC
CGCAACCTCGCTCTGGGGTCCGAAGTAGCTTTTGAACTCAACGGTCAAGAGACATTTTTG
CGGGTTATCGGTGTGTATAAAGAAGCCGCAGCAGGTGGACTTGTGGGAAGCAATCCAACC
GTCCACACCTACACCCCATATACGGTGGCCAATGACATGACCCACACGGAAGATGGATTG
AACACGTTAAGTATCCGTGCAGCTCAGGGCGTAGACCAGGATTCACTTAAGGGTTCACTG
CAAACCTACTTGGACGCGCTGTACGCCAACAATGACTGGCACCACGTTGCCATGTTGGAC
TTCCGTAAACAGATCGAAGAGTTCAACACCATTCTCGGCGCAATGAGTTTGGGTATCTCA
GCCATCGGCGGAATTTCCTTGCTTGTCGGTGGCATCGGAGTGATGAACATTATGTTGGTG
TCTGTCACCGAGCGAACCCGCGAAATCGGTGTCCGAAAAGCCCTCGGCGCTCGTCGACGT
GACATTCGCCTGCAATTCGTCGTTGAAGCCATGATCATTTGTTTCATCGGTGGCATCCTC
GGCGTGCTTTTGGGCGGCATTTTGGGATTGATCATGTCCAGCGCTATTGGCTACATTTCC
TTGCCACCACTGAGTGGAATCGTGATCGCCTTGGTATTTTCCATGGCTATCGGCCTGTTT
TTCGGCTACTACCCCGCCAACAAGGCAGCAAAGCTCGATCCAATTGACGCCTTGCGTTAT
GAG
>RXN00525-downstream
TAAAAGCCTCGTTTTTAAGGTAG
>RXN00702-upstream
TGGGGACGATGCCAGGATTCTTGACGCCCGGCTTGCCGAAGAACAAAGTGAGGGTCAAGC
GTCGAAAAGCAATAAATAGCCCAGAAAGGGCCGAAGTTTA
>RXN00702
ATGAGTGGTCCTTTTAGCGCGCGCACTGGGTGGTGGACGGAGCCCGTGCTGGAACTGGAA
AGCGTCGGTGCCTCGTATTATGACGATGAGCGCACGCTGGCGGCGCCGCAGATCAGCGAC
GTGAATCTGACGCTTTTTGAAGGCGAAATCCTGCTGGTTGTGGGGCGCACCGGCTCCGGC
AAATCGACGCTGCTGAACGCGATGTCCGGCGCGATGCCGCATGCGACCGGCGGCCGACTT
GATGGGGGCGTGCGCGTGGTCGGCCGGGATACGCGTGATTTCCCACCACGCATGCTTTCC
GACGTGGTCGGCGTCGTTGGGCAAGATCCGGCGGCAAGTTTTATCACCAACACGGTTGAA
GAAGAACTTGCCTACAGCATGGAGCAATTAGGGGTCCCACCTGCGGTCATGCGCAAGCGC
GTAGAGGAAACCCTTGATCTTTTAGGCATCGCGGAGCTGCGATACGTGCCATTGGCGGAA
CTATCTGGTGGTGAGCAGCAGCGCGTGGCGATTGGCGCGGTGCTGACCACTCGCCCCGCG
CTGATTATCTTGGATGAACCAACCAGCGCTTTGGACCCTAATGGTGCCGAGGATGTGCTG
GCAACCGTAACCAAGCTGGCTCATGACTTGGCGATGACCGTAGTGCTTGCTGAACACCGC
ATCGAGCGCGTACTGCAGTACGTGGACCGCGTGGCGCATGTGGGCGCTGATGGGCACGTC
ACTGTTGGGACGCCGGAAGAAATCATGGCTGATTCTGATGTGGCACCACCCATTGTGGAA
TTAGGACGCTGGGCTGGCTGGGCTCCCCTACCGCTATCGATCCGCGATGCACGCGCACAC
TCCGGTGACATGCGCAAACGCCTGTATCAGGGTGGTTTAGTGGTGAACAAATTACACAAC
CACGCTGTCCAGCCAGTTTTGATCGCCGAAGATATCATGGTTGATTTCCGCGAAATCCGT
GCCGTTGACGGCGTGAACTTGAATCTCAACTCGGGTGAAATTACCGTGCTCATGGGCCGA
AACGGCTGCGGAAAATCATCCCTGCTGTGGGCTTTACAAGGTTCAGGGACTAGAAATCAG
GGCTCGGTGCAGGTGCTTGATGAGGCCGCGGGATTTTCGTGGACAGAGCCCAAAACTTTA
AAGCCCGGCAAGCGGCGCAATCTTGTGTCCATGGTTCGGCAAACACCGACCGATATTTTG
TATGAATCAACCGTGCATGCAGAGCTGGCACGCTCTGATAAAGATGCCGCAGCACCCGCC
GGCACCACGCGGGAAATCCTGGATTCACTGGTCCCGAATATCCCGGACCATCTCCACCCA
CGTGATCTATCAGAAGGCCAAAAGCTCTCCCTCGCGCTGTCCATCGAACTCGCCGCAAAA
CCCCGCGTGGTATTTTTCGACGAACCCACCGGCGGCCTAGACTACGACGGCAAGAAATCC
CTCGCCGGCTCCTTCGAACAACTCGCAGACGACGGCGACGCCATTTTGGTGGTCACCCAC
GACGTGGAATTCTCTGCACTGTGCGCCGACCGAGTGTTGTTTATGGCCTCTGGAAAGATC
ATCTCCGATGGCACAGCCGTAGAAATCCTCGCCGCATCACCGGCTTACGCCCCACAAGTC
GCAAAAATCACCGCCGGCATCCAAGAGGAATCACACTGGCTCACAGTCTCGGCCGTGAAA
GGTGCGGTAGGGCATGGTGAAATCTCA
>RXN00702-downstream
TGATCAACGCCATCACACTCAAG
>RXN00726
AACGCGGGTCGCTTGTATGTGGATGGCGATCTCATTGGCTACCGAGAGCGCGATGGCGTG
CTGTACGAAATCTCTGAGAAGGACGCCGCGAAGCAGCGCTCCGATATCGGCATGGTGTTC
CAGAACTTCAACCTCTTCCCCCACCGCAGGGTGATCGAGAACATCATCGAAGCTCCCATC
CACGTGAAGAAGCAGCCCGAAAGCAAGGCCCGCGCACGTGCCATGGAGCTGCTTGAGCAG
GTCGGCCTCGCCCACAAGGCGGACGCCTACCCCGTCCAACTGTGGGGTGGTCAGCAGCAG
GGCGTTGCAATTGCCCGCGCCGTCGCCATGGAGCCAAAGCTCATGCTTTTCGACGAACCC
ACCAGCGCTTTGGACCCTGAACTCGTCGGTGAAGTCCTGCGAGTGATGAAACAGCTCGCC
GACGACGGCATGACCATGCTTGTTGTCACCCACGAAATGGGCTTCGCCCACGAAGTCGCC
GACCAGGTCGTGTTCATGGCCGATGGAGTTGTCGTTGAAGCCGGAACCGCCGAAGAAGTT
CTGGACAATCCAAAGGAACAGCGCACCAAAGACTTCCTGTCTTCTCTGCTC
>RXN00726-downstream
TAACCTTTTCGGGTCTTAAAAAA
>RXN00732
AATCACCTCCTCCTACTCCCCACGGTAAAGGCAGACATCATTGACAATGGTGTGGTCACA
GGTGACATCGGCTATATTTGGCACACCGGTGGAATCATGCTGGCCCTGACATTAGTCCAG
GTTGCCTGCGCTATCGCCGGTGTTTATTTCGGTTCCAAACTATCCATGAGAGTGGGCCGC
GATCTGCGTTCGGCGATCTTTGGCAAGGTAGTGAACTTCTCTGAGCGTGAGATGGGTCAG
TTTGGCGCACCGTCGCTGATCACCCGAAACACCAACGATGTGCAGCAGGTTCAGATGCTG
GTGCAGATGACCTCCACTTTGATGATTTCCGCCCCGATGCTGGCCATTGGTGGCATCATC
ATGGCGGTGCGTCAGGATCTTGGTTTGTCTTGGCTGATGGTGGTCAGTATTCCGGTGCTC
ATCATCGTGGTGGCGCTGATCATTGTGCGCATGGTTCCGTTGTTCCAAACCATGCAAAAG
CGCATTGACCGCATCAATCAGATTATACGCGAGCAGCTCACCGGTATCCGCGTGATCCGC
GCGTTCGTGCGTGAAGATGTGGAACGCGAACGATTCAGCACTGCTAGTAAAGATGTCGCT
GATATCGGCGTGCGCACGGGTAAGCTGATGGCGTTGATGTTCCCTGCCGTGATGCTGATC
ATGAACCTTTCTGCCGTTGCTGTGATTTGGTTTGGTGCTTTCCAGGTGGAATCCGGCGAG
ACGCAGATCGGTACGCTCTTTGCATTCTTGCAGTACATCATGCAGATCCTCATGGGCGTG
ATGATGGCAGCGTTCATGTTTGTGATGGTTCCGCGCGCTGCCGTTTCGGCTGATCGCATC
GGTGAGGTTCTGGAAACCACACCGTCTGTGCAGGCGCCAGAAACACGGGCGCAGCCGTCG
ACAAGCGCTGGCGAAATCGTGTTCAACAACGCGACTTTTGCCTACCCCGGCGCGGATGAC
CCCGTGTTAAATAATGTGAGCTTCCGCGTTGCGCCTGGTAGCACGACGGCGATCATCGGC
TCGACGGGTTCGGGTAAGACGACGTTGATCGGGCTGGTTCCTAGGCTTTTCGACGTCACC
GAAGGCGACGTTACCGTCGATGGCACCGATGTTCGTGAATTTGAGCCGCTGAAGCTGTGG
GATCGGATCGGTCTTGTTCCGCAGAAGTCGTTCCTGTTTTCTGGAACGATCGCCAGCAAC
CTGCGTTATGGCAATGAAGATGCCACGGAAACGCAGCTGTGGCAGGCGCTTGCAATTGCT
CAGGCGGCGGACTTTGTGCGTGAGATGCCAGAGGGTCTTGATTCTGAGATTGCTCAGGGT
GGAACCAATGTTTCTGGTGGTCAGCGCCAGCGACTAGCCATTGCCAGGGCGTTGTTGAAG
CAACCTGAGATCTATATTTTCGACGATTCTTTCTCCGCCCTCGATGTGAGCACAGACGCC
GCTCTTCGCCGAGCGCTGAGCACCAACCTGCCGGATGCAACCAAGTTGATTGTCGCCCAG
CGTGTCAGCACGATTCGAGATGCCGATCAGATTGTGGTGCTTGATAACGGCGAGGTTGTC
GGTATTGGAACGCACACGAATTTGCTGAAGACGTGCGGTACCTACCGTGAAATTGTTGAA
TCCCAAGAGACTGCGCAGGCGCAATCA
>RXN00732-downstream
TGAGTAATACTGCAGGCCCCCGC
>RXN00759-upstream
TCACCTTGAACACTTAAAAGATAACTTCATCCGGCGCTTTATTAGCTTGAAGCGCCCCGC
ACCATAATCCATTCCCCAGCAAGCAAGGACACCCACGCTC
>RXN00759
ATGCTTCGTTACGTCGGGCGACGTTTGCTCGAAATGATTCCGGTGTTTTTCGGAGCGACC
TTACTGATTTACGCCCTCGTGTTCGTCATGGCTGGTGACCCAGTGCAGGCATTGGGAGGT
GACCGCGGCCTAACCGAGGCTGCGGCCGAGAAAATCCGTCAAGAATACAATCTTGATAAA
CCCTTCATCGTTCAATACCTCCTGTACATCAAGGGCATCTTCGTCTTAGATTTTGGAACA
ACGTTCTCTGGTCAGCCAGTTATTGATGTGATGGCCAGGGCCTTCCCCGTCACCATCAAA
CTCGCCATCATGGCCCTGCTGTTTGAATCAATCCTCGGCATTATCTTTGGTGTCATCGCA
GGTATTCGCCGCGGAGGAATCTTCGACTCCACCGTGCTGGTCCTTTCTCTGATAGTCATC
GCAGTCCCCACCTTCGTCATTGGTTTCGTGCTGCAGTTCTTAGTCGGCGTGAAATGGGGC
TTACTGCCCGTCACCGTAGGTTCCAACACATCAATAACGGCGCTGATCATGCCGGCTGTC
GTACTGGGTGCAGTATCGTTCGCCTACGTTCTTCGCCTCACCAGACAATCCGTGAGCGAA
AACGTCCGCGCTGATTACGTTGGAACCGCTCGAGCAAAAGGGATGTCCGGATTGAACGTG
ATGAACCGCCATGTGCTTCGAAACTCACTGATTCCCGTTGCCACCTTCCTGGGCGCCGAT
CTCGGTGCACTGATGGGTGGAGCGATTGTCACCGAAGGTATCTTCGGCATGAACGGTGTC
GGTGGAACGCTCTACCAGGCCATTTTGAAAGGTGAACCCACCACGGTTGTCTCCATTGTC
ACTGTGCTGGTCATCGTCTACATCATCGCCAACCTTCTCGTGGACTTGATCTACGCCGTT
CTCGATCCGAGGATCCGCTATGCC
>RXN00759-downstream
TAATAATGAATTCCACACAAACC
>RXN00808-upstream
CGCGATGTCGCACCGGCACGTTAGAGTATTGAGCATGAGTCGATTGCTTAGAGCATTGAA
ATGGCTGTGGGGCACATCGTGGCCGCTGTATGCTGCGACG
>RXN00808
GTGCTCGGCACGAATGTGTTTGGTGCGCTCGCAGTAATGCTGTTTGTGCGCTTCCTCATT
CCGCAGCCAGATGCTTCAAATTTCAACGCTGAGATCTCGTATCTGCCAGCTGTTGGTTTC
GCATACCTGGCGTTCGCCATTGTCGCGGGCATGCTGGTGACATTTTTGATGTTCCGCCCG
GTGCTTGATTGGCAGCGAAGCCCTGAAGATCATGACCGAAATATGGTGCGCAACTTGGTT
ATGCGCATCCCCATCTACCAGGCAATTCTGTGCGCAGTGGTGTGGTTAATCGGCATTGCA
ATTGCAACGTTGATTTCGGCCAGTGTGTCTACCAGTTTGGCGCTGGTCGTGGCGTTTTCC
ACGTTGATGGCTGCCGCAATCGTCGTGCTGCTCACCTACCTTGAGGCTGAGCGTTTGGTG
CGTCCGGTTGCTGCGTCTGCCCTGGCGCGTCGATTTGAGGATTCCACGCTGGAACCACCT
GTGAGCCAGCGCTTGCGTATGACGTGGTTGCTGACGTTGGGCATTCCAGTGATGGGAATT
CTGCTGGTTATTTGGGGCTACTCGCAGGGCATTTTCGGGTCTGATGCCTGCGGAATTATG
CCTGCCATCGCAGCGCTCGCGTTTGCATCGTTGGTCACGGGTTACCTGGGCAACCGGCTT
GTGGTGTCCTCTGTGGTGGATCCGATTCGGGAACTTCAGGAGGCCATCAACAGGGTTCGT
CGTGGTGAAAACGATGTGGAGGTTGATATTTATGATGGCTCTGAGATCGGTGTGCTTCAG
GGTGGCTTCAATGAGATGATGCGTGGCCTGCGTGAACGTCAGCGCGTCCGTGACCTTTTC
GGTCGCTACGTGGGCGCTGAAGTGGCCAAGCGTGCGCTGGAGGAACGCCCCACTCTGGGT
GGCGAGGACCGTAAGGTTGCCGTGTTGTTTGTCGATGTCATCGGCTCCACTACCTTTGCC
GTCAACCACACTCCTGAAGAGGTTGTGGAGGCGCTCAATGAGTTCTTCGAGCACGTCGTG
GAGGTTGTGCACCGCAACAAGGGTGTTATCAACAAGTTCCAGGGTGACGCGGCGTTGGCG
ATTTTCGGCGCTCCCCTGCCCCTGTCTGATGCCACCGGTCATGCGCTTGCGGCTGCCCGT
GAGCTCCGCGCAGAGCTGAAAGATCTCCAGCTCAAGGCCGGAATTGGTGTGGCTGCTGGC
GATGTCGTTGCTGGTCATATCGGCGGTCACGCGAGGTTTGAGTACACTGTGATCGGCGAC
GCGGTGAACCAGGCTGCGCGCCTGACGGAGATCGCGAAAACGACCCCAGGCCGCACCGTC
ACCAACGCTTCCACGCTGCGTGAGGCCAACGAGGCGGAGCAGGCTCGCTGGACGCTCATG
AAGTCCGTGGAGCTGCGCGGACGTAGCCAGATGACGCAGATTGCGCGGCCTATTCGGCCG
ACGTTGGCGGATAGGTCC
>RXN00808-downstream
TAATACGCTTTTCGACGCAAAAA
>RXN00828-upstream
CGGTGATCACCGGGCCGAATGGCGCTGGAAAATCCACACTTGCGCTGACCATGGGTGGAT
TGCTTCCGCGAAAAGTGGGCAGCTGGAACTGTCTGACACG
>RXN00828
GTGCGCGGCGGCGTTAACACGCCCCCGCACAAGTGGCGTTCAGCTGATGTAGCTGCACGT
ATTGGCACTGTCTTTCAGGATCCAGAGCACCAATTTGTGGCGCGCACTGTGCGTGATGAG
GTAGAAATTGGGCCGAAAATCATGAAAGTCGATGCAAGCGAGCGGATCGAGGAGCTGCTT
GATCGTTTGCGCCTCCGCCACTTGGAAAACGCCAATCCGTTTACCTTGAGTGGTGGAGAA
AACCGCCGCCTATCTGTGGCGACAGCCTTGGTGGCAGCACCGAAACTTCTCATTTTGGAT
GAGCCTACGTTTGGCCAAGATCCCGAGACCTTCACAGAGCTGGTGACGATGTTGCGTGAA
TTAACAGACAACGGAATCAGCATTGTGTCGGTAACCCATGATCCTGATTTCATCGCAGCG
GTGGGCGATCACCACATTGAGGTGAGCGCGAAG
>RXN00828-downstream
TGAACCTGCTGATCAAAATTAAT
>RXN00832-upstream
GAGATTGTGCTAGGTTCTGATGAGGCTTCGGGACGACCCGAAGAAATCTATGACAGCCTG
GGAACGGCCCAGAGTTCTTAAGAAAGTTTGACTAGAGAAC
>RXN00832
ATGCCGTTTTCTTGGCTAAAACCAATTGATTATGCCCGCATCTTTGTCGGCTGGGCATCG
ATTTTTATCATCCCCCTCATCACACTGCCATCAATTATTGAGTTGGCGCTGATCGTGGCA
GTCATCCTATTCTGCGCATTTGGCGTGGTGAAGATGGCGGAGCGTTTGGCTCATATTTTG
GGTGATCCTTTTGGATCGTTGATCCTTACCTTGTCGATCGTGATCATTGAAGTGATTTTG
ATCTGTGCGGTGATGCTGGGGCCTGCTGATTCAACCACTGCTGGTCGGGATTCCGTGATG
GCAGTGTCCATGATCATCATGGGTTTGGTCGTGGGATTGTGCCTACTCATTGGTGGTTTA
AGGCATGGAAGCATGCCACACAATGGGGTGGGAACTCCGACCTACTTGGTGCTGATCGCA
ACTTTTTCCGTAATCGCCTTTGCGGTTCCAGCTTTCAGGGGAGAATACTCCACTGGGCAG
GCACTTGTTATTTCAACACTGACAGCAGTGGTGTACGGGTTCTTCCTGTTTCGCCAAATG
GGTGCCCAAGCTGGTGAATTTCAAGAGGTCGAGGTCGCAGAAAAGGCAGACGACGCAGCA
AAATGGGAGGTCCCATTTAGAGGCTTAATCTTGATTATCACTGTGCTCCGCATCGTGTTG
CTGTCCCATGACATGGCCACGGTGATGGATGAAGTCCTGGCAAGCCTTGGTGCACCCGTA
GCAATGGCTGGATTAATTATTGCCACCATTGTCTTCTTGCCAGAGACCATCACCTCCTTG
AAAGCTGCGTGGACAGGAGAGATTCAGCGAGTAAGCAACCTCGCGCATGGAGCCCAAGTA
TCAACGGTGGGGCTGACAATCCCAGCTGTTCTAGTGATCGGCGTGATCACAGGTCAAGAT
GTAGTTTTGGGGGAGACCCCGATCAACTTGTTGCTGCTGGGAACCACCATTGCGGTGACA
GCCATTGCGTTTAGCTCCAAGAAAGTCAGTGCTGTGCATGGCTCGGTGCTGCTCATGCTT
TTCGGTGTTTACATGATGAGCATGTTCGCC
>RXN00832-downstream
TGATTTAGGTAGCCTGGTGGGAA
>RXN00934-upstream
CCAACCCCTGTGGTTTGGTGATTTGGATCCGGAGCGTCTCAAGCGCTCTAGGGAGCAGAC
AAATGTTCACAAACCGGTGGCATTACAGGAGGACAATTAG
>RXN00934
GTGCGAATTGGAATGGTCTGCCCGTACTCCTTCGATGAGCCGGGCGGTGTTCAAGCGCAT
ATCCTTGACTTAGCGCGAACCTTCATTGCCCAAGGCCATGAGGTTCAGGTGCTTGGTCCG
TGTAGTGCGGATACGCAGGTGCCCGATTTCGTGGTGCGCGGTGGTGGCAGCATCCCGATT
CCGTACAATGGCTCGGTTGCCCGCTTGAGCTTTGGGCCGAAAATGTTCAAGGCCGTGCGC
ACGTTCCTCCGCGAAGGCAACTTCGATGTGCTGCATATCCATGAACCGAATTCACCAAGT
TTTTCCATGGCGGCGCTACGCTTTGCGGAAGGCCCCATCGTTGCTACTTACCACGCCTCC
AGTAGCGGATCGAAGCTGCTCAAGGCTTTCTTACCAGTGCTTTCGCCCATGCTGGAGAAA
GTGCGCGCAGGCATCGCCGTGTCTGAAATGGCTCGGCGCTGGCAGGTGGAGCAAGTCGGC
GGCGATCCGGTGGTGATCCCCAACGGGGTAGAGACCTCGATGTTCAAAGCCGCGCGCCAA
ATCGAACCGAATGATCCTGTAGAGATCGTCTTTTTGGGTCGCGTCGATGAGTGCCGCAAA
GGCCTCGACATCGTGCTGCGCGCTCTGACCAGGCTGGATCGCCCGTTTACCTGCACGGTC
ATTGGCGGCGGCACCCCGCGAGAAGTCGCCGGCATCAACTTTGTGGGCCGCGTCAGCGAT
GAGGAAAAGGCAGCAATCTTAGGTGGCGCAGACATCTATGTCGCACCCAACACCGGCGGC
GAAAGCTTCGGCATCGTGCTAGTTGAAGCGATGGCGGGGGGATGCGCTGTCGTCGCCAGC
GACCTAGAAGCGTTCTCCCTGGTCACCGATTCTGAAGCCGCACAGCCAGCGGGCGTGCTA
TTTAAAACCGGCTCAGACGCCGACCTAGCCAAAAAACTTCAAGCGCTTATCGACGACCCC
TCCTCCCGTTCCACGCTTATCGCCGCGGGGCTAAAGCGCGCAAACGCCTACGACTGGTGG
ACAGTATCGACCCAGGTCATGGCAGTCTATGAAACCATTGCGATCGACAAAGTGAGGCTT
GGA
>RXN00934-downstream
TGACCCTTGTTTACCTCCTCATC
>RXN00939-upstream
GAATTCCACCCTGGACAAAGATCATCCCACCGTGTCGAAGACGCCCAGG
>RXN00939
ATGACAAGGCAAAAAACCCAGCCGTTCCTGGAGAAATTCTCGAAGTACTACACCCCCGGC
GTCATGATCGCCGCCGTGGCCGTCGGCCTGATCACGCTGAACGTTGAACTGGCCCTGACC
CTGCTGGTCATCGGGTGCCCGGGTGCCCTGGTCATCTCGATCCCGGTCTCGATCGTCGCC
GGTATCGGCCGCTCCGCCAAGGACGGCGTCCTGATCAAGGGCGGGGAATACGTGGAGACC
TCCGCGAAGGTCGACACCGTAGTCGTCGACAAGACCGGCACCCTGACCAACGGCCGCCCC
GAGCTGACCAACGTCGACGTCCTTGACCCCGCCTACTCGGACGATGAGGTGCTCACCCTG
GCCGCCCGCGCGGAAACCGCCTCCGAGCACCCCCTGGCCGAGGCCATCATCCGCGGCGCG
GAGAACAGGGGCTTGACCGTGGCGATGGTAGAAAAGGCCGAACCGGTCGCCGGCCGCGGC
ATCCGGGCTGACGTGGACGGTGCCACCGTGGCCGTGGGCTCAGCCGACCTGCTCGATCAC
ACCCCGGATAACACCCGCATTCTCGAGCTCAACGAACAGGGCAGGACCGCCATGTACGTC
GGCATCAACGGCAAGGCCGTGGGCATCGTCGCTGTGGCCGACACCATCCGAGATGATGCC
CCGGCCGCGATCAGGTCCCTGCACAATAAGGGAATCCGCGTGGTCATGGCCACCGGTGAT
GCCGAACGCGTCGCCCGCAACGTCGCCGCCGAGCTCGGTGTCGATGAGGTGAGGGCAGAA
CTGATGCCTGAGGACAAGCTCGAGATCGTCAAGGAGCTGCAGGCGCAGGGCCGGGTCGTG
GCCATGGTTGGCGACGGTGTCAATGACACCCCGGCACTGGCCACCGCGGACATCGGTGTG
GCGATGGGTGCGGCCGGTTCGCCTGCCGCCATCGAGACCGCCGATATCGCCCTGATGGCC
GACAAGCTGCCGCGGCTGCCCTACGCCCTGGGTCTGGCCCAGCGCACGGTGCGCACCATG
CGGGTCAACATCGGCATCGCCCTGCTCACTGTCACGATCCTGCTGGCCGGTGTCCTGCTC
GGTGGAGTGACCATGTCGATTGGCATGCTCGTCCACGAGGCCTCCGTCCTGCTGGTCATC
GCGATTGCGATGCTCCTGCTGCGCCCCACCCTGAAGGAAGACAAGGACAAGGCAGACGTC
AGTACTGCTGACGCCGCGAAGGAGACGCTGAGCGCC
>RXN00939-downstream
TAACGACACAATCGCCACAGCCA
>RXN00960
ATGGCTCGGCATTGTTGCAGCAATCGCTACGCGTCCACCGTCTTCTCCGGTCTGATCGCC
TACGGAGCATCCCAAGCGCTCTACCCATGGCTGCTGAAAGACCACCAAAGCGTCACCGAA
ATCGACCTTGATGCAGGTGCCCTGCAGCCCTACTTCAACATCGAGATGCCACCACCATTT
GAAGTGATGACCGCACTGCTGCTGGCATTCTGCCTCGGCCTGGGCATGGCTGTAATTAAA
TCAGACACCCTGTTCAAGGTAACCCGCGAACTCGAGCGCGTAGTCATGAAGACCATCACC
GCCTTTGTCATCCCACTGCTGCCACTCTTCATCTTCGGCATCTTCCTCGGCATGGGCATG
AACGGTGGCCTCCTGGAGATCATGTCCGCCTTTGGCAAGGTACTGATTCTCGCCGTCGTG
GGAACCCTGCTCTTCCTAGCCATCCAGTTCATTATCGCTGGTGCAGTATCCAAGAAGAAC
CCATGGAAACTGTTCAAAAACATGCTCCCTGCATACTTCACTGCACTGGGCACTTCCTCT
TCAGCGGCAACCATCCCAGTGACCTACCAGCAGACCCTGAAAAACGATGTTGATGTCAAC
GTCGCAGGCTTTGTTGTCCCACTGTGCGCCACCATCCACCTAGCTGGATCGATGATGAAG
ATCGGCCTCTTCACCTTCGCTGTTGTCTTCATGTACGACATGGAAGTAGGCGTCGGCCTC
TCCATCGGATTCCTCCTCATGCTGGGCATCACCATGATCGCCGCACCAGGCGTTCCCGGC
GGAGCCATCATGGCAGCAACCGGCATGCTGGCCTCCATGCTCGGATTCAACACCGAACAA
GTCGCCCTCATGATCGCCGCTTACATCGCGATTGACTCCTTCGGCACCGCAGCAAACGTC
ACCGGCGACGGCGCAATCGCAGTCATCGTGAACAAATTCGCCAAGGGCCAGCTGCACACC
ACTTCGCCAGATGAAATCGAAGAAGACGACCGCGTTGCCTTCGACATCACTCCATCGGAT
GTGGAACATCACAAG
>RXN00960-downstream
TAGAAACCCGCATTTTCTGTAGT
>RXN00980-upstream
AGAGAGAAAGGGAGAAATCATGAAAACGTGGAAGACCTGGGGGGTCGTCGGAGCTTGAGG
CCTCTTGATTATTTTGTCGTGGTTGAGTTCATCGAGCCCG
>RXN00980
ATGCTGGCAGATGCATTCATGATCGCGGCTGCAATTGTTGGAGGTTGGCCGATCGCGCAG
TCTGCATATCAAGCACTTCGCATTCGAATGGTGTCGATTGACTTACTGGTCGTTGTGGCT
GCCGTTGGTGCCATGTTCATCAACAACTATTGGGAGTCTGGGGCGGTGACGTTCCTCTTT
GCCCTTGGCAAGGCACTGGAACGCGCGACAATGAACCGCACACGAAAAGCACTATCGGAT
CTGGTGGATGCAGCTCCAGAAACTGCAAGAAGGCTCAACGCGGATGACTCAACAGAGGTA
GTTGAGCTGTGGGAGCTTGAGCCCGGTGACATCGTCTTGGTACGCAATGGCGAACAAATT
CCCGTCGATGGAAACGTGATTGCGGGTGTCGGTGGAATTGATGAATCCAACATCACGGGT
GAATCAATGCCGGCTGAAAAGGGTCAAGGCTCTGATGTGTATGCAGGAACCTGGCTGCGA
TCTGGTGTTTTGAGAGTCGAGGCAACAGGAATTGGTTCAGACTCAACTTTGGCAAAAATC
ATTCACCGCGTTGAAGACGCCCAGGATGACAAAGCCGGCACACAAACATTCTTAGAGAAA
TTCTCTAAGTGGTACACCCCGGGCGTCATGATCGGCGGCGCAGTGGTGGGACTTATCACC
TGGGACGTAGAACTAGCACTGACGCTCTTAGTGATCGGCTGCCCCGGCGCGTTGGTTATC
TCCATCCCGGTGTCCATCGTCGCAGGCATCGGCCGTGGTGCACGCGATGGCGTGCTGATC
AAGGGTGGAGAATACCTAGAAACCGCGGCGAAAGTCGACGTCGTTGTCGTGGACAAAACT
GGAACGCTGACGACCGGCCGCCCAGAACTCACAGACGTAGAAGTCATCGAGCCCGCCTAC
AGCCAGGGCGAGGTGCTGGAGCTCGCCGCGCGCGCCGAGACGGCTTGAGAACATCCGCTT
GCCGAGGCCATCATGCGTGGTGCCCAGGATCGGGGGCTGTCCACAAGATTGGTGGAAGCA
GCTGAAAACATCACCGGCCGAGGCATTATCGCAAATGTTGATGGACAGGCAGTTGCTGTT
GGATCTGCTGAGTTACTTGATCATGAACCAGACTCGACCAGGATCCTGGAGCTAAATGCC
GAAGGAAAGACCGCGATGTTTGTCGGAGTGAACGGACACGCCATTGGAATCGTGGCCGTC
GCCGACGCCGTTCGTTCAGATTCTGCCTCAGCAATCGAATCGCTGCATAAGGCGGGCATT
CAAGTTGTCATGGCGACTGGCGACGCTCACCGCGTTGCACAAAACGTGGCCTCCAAGCTG
GGAGTGGATGAAGTCTACTCAGAGCTACTCCCTGAACAGAAATTAGAACTGGTGCGTGAT
CTGCAAGCTGCCGGCAAAAGGGTCGCGATGGTGGGTGACGGAGTCAACGACACCCCAGCA
TTGGCAGCTGCTGATATCGGAGTAGCGATGGGCGTGGCAGGTTCCCCTGCAGCCATTGAA
ACCGCTGATATCGCACTCATGGCGGATCGTCTCCCACGGCTGGCACATGCAGTGACCTTG
GCAAAACGCACCGTAAGAACCATGCGCATCAATATTCTGATTGCGTTGGCTACCGTGATG
GTGTTACTAGCTGGCGTCCTATTTGGCGGAGTTACCATGTCGGTTGGCATGCTCGTTCAC
GAAGGAAGCGTGCTGCTTGTTATCAGCATCGCCATGCTGTTGCTGCGTCCAACACTTAAA
GAAGATGCTGCGCAAGCAAGTGATATTAAACGCTCGGAAATACAACAGATCGCA
>RXN00980-downstream
TAACCAATGGCTGGGTACTGATG
>RXN01000-upstream
CTTTCTATGCCTACGCGGATGTTTCCGTGATCATTCTGGAAATGCTCATCGTGGTGATTG
TCATTGAAGTAATCTCCAACGCACTTCGAAAGAGGCTGGT
>RXN01000
ATGAGCACCTTAACCTCTCACCGCACAGTACCGGCCCCCAGCTCTCCCCCGGCGCGCCCC
AACAAACTGGCGCGCAATATCGTTGCAATTGTCGCTGCGCTGATTGTCCTTATAGCTACC
GGCACGCTCAAGATCGAGTGGAATGAGCTTCCGCAGATGCCCGCGCAGGTGTGGCATTAC
TTAGAGCTGATGTTTAGCGATCCCGATTGGTCGAAGTTTGGCCGCGCCGTCCAGGAAATG
TGGCGTTCCATCGCCATGGCGTGGTTGGGTGCCATTTTATGCGTGGTGGTCTCTGTCCCT
CTGGGAATGTTGGCTGCCCGCGGGGTGGGACCTTATTGGCTGCGTACCGTTTTACGGTTC
GTGTTCGGGGTGATTCGTGCGTTCCCCGAAGTGGTTATCGCAATTATTTTGCTAACTGTC
ACCGGCCTAACTCCTTTTACTGGTGCGCTCGCATTGGGTATCTCCGGTATTGGACAACAG
GCAAAGTGGACCTATGAAGCCATTGAGTCCACTCCCACCGGCCCGTCAGAGGCAGTGCGT
GCAGCGGGTGGAACTACGCCGGAGGTTCTGCGGTGGGCGTTGTGGCCACAGGTTGCGCCA
TCCATTGCATCTTTTGCCCTGTACCGCTTTGAGATCAACATGCGTACCTCTGCGGTATTG
GGCATCGTTGGTGCAGGTGGTATCGGTAGTATGGTTGCCAATTACACCAACTACAGGCAG
TGGGACACCGTGGGCATGCTGCTCATCGTCGTGGTTGTCGCAACGATGATCGTCGATCTC
ATCTCCGGCACCATCCGCCGGCGCATCATGAAGGGGGCTAGTGACGGTGTCGTGGCACCA
AGCAAC
>RXN01000-downstream
TGACGCTCGACCAAGCATCCGCA
>RXN01002-upstream
GACTGCTGATACCGCACAGGATGAAATGACTCGTTACGGCGAGATGCTGAAGAAGTTCTC
GAACTAATTTCCCTGTTTCCAATACTCAAGGTGTGCGCAT
>RXN01002
ATGAATTCTGATGCTTCGGCTACCACCAACTCCTGGGCTATCAACTTCGACGATGTGTCG
GTGACGTATCCCAATGGGACGAAAGCCCTCGATGATGTTTCCCTCACCATCAATCCCGGT
GAGATGGTTGCCATCGTGGGTCTGTCAGGATCGGGTAAATCCACGCTGATTCGCACGATC
AACGGTCTTGTCGGGGCTACGGAAGGCAGCGTGACGGTGGGGCCGCATCAGATCAACACC
TTGAAGGGGAAAGCACTGCGTGATGCCCGTGGGCAGATCGGCATGATTTTCCAGGGGTTC
AAGCTGTCGGAACGCAGGAGTGTGTTCCAGAATGTTTTGGTGGGCCGCTTGGGGCACACA
GGGTGGTGGCGTAACCTCGTCGGGTTTCCCACGGAGCACGAGAAGCAGATTGCTTTTCAC
GGGTTGGAGTCCGTGGGCATTTTGCACAAAGTGTGGACGCGAGCTGGTGCTTTGTCGGGT
GGACAGAAACAGCGCGTTGCTATTGCGCGCGCCTTATGGCAAGATCCGTCTGTCATGCTG
GCAGATGAGCCTGTGGCAAGCCTTGATCCGCCAACCGCGCATTCCGTGATGCGCGATCTA
GAAAACATCAACAACGTGGAAGGCCTCACCGTGTTGGTGAACTTGCACTTGATTGATTTG
GCTCGTCAATACACCACAAGGCTTGTGGGTTTGCGTGCCGGCAAGCTGGTCTATGACGGT
CCTATCTCTGAGGGCACCGATAAAGACTTTGAAGCTATCTATGGTCGCCCCATCCAGGCT
AAAGACCTGCTAGGTGATCGCGCA
>RXN01002-downstream
TGACCACGCCTTCTTCTACACTT
>RXN01141-upstream
AAAGAACACTCGGTATGGCACCTGATTTAAGGATGCTGCAATCGTGACACATATCCTCTT
CGACAGCAGGCGTTTTCTGCAACTGGGCGCTTTTGCGTCC
>RXN01141
TTGAGCACCGCATTGGCCGGAGCGGCCCGCTACGTGACGTCGACAAGCAATAATGAACCT
GCGGATAACACTCCCCTGACCATTGGCTACGTGCCTATTGCGGGCTCGGCGCCGATTGCT
ATCGCAGATGCGCTAGGGCTGTTTAAGAAACACGGCGTGAATGTCACGTTGAAGAAGTAC
TCAGGGTGGTCCGACCTGTGGACCGCCTATGCAACAGAGCAGCTTGATGTTGCGCACATG
CTGTCGGCGATGACTGTGGCGATTAATGCTGGAGTGACCAACGCGTCGCGCCCGACGGAG
CTGTCGTTTACCCAGAACACCAATGGGCAAGCAATTACCTTGGCGTCAAAGCACTATGGT
TCCGTCAATTCAGCGGCGGATCTTAAAGGCATGGTGCTGGGAATTCCTTTTGAATATTCA
GTGCATGCGCTGCTCCTGCGCGATTATCTCGTCTCAAACGCAGTTGATCCCATCGGCGAT
CTTGAGCTTCGCCTGCTCCGACCTGCCGATATGGTCGCACAATTGAGAGTTGAGGGCATC
GATGGATTCATTGGGCCTGGGCCGTTTAATGAACGCGCCATCAGCAATGGCTCCGGCCGG
ATTTGGCTGCTGACCAAACAACTGTGGGACAAACATCCATGCTGCGCCGTGGCGATGGCC
AAAGAGTGGAAAGCTGAACACCCCACGGCGGCTCAGGGTGTGCTTAATGCGGTGGAGGAA
GCCTCCGCAATTTTGAGCAATCCGGCACAATTTGATTCCTCGGCACGCACGCTGTCGCAG
GAAAAATACCTCAACCAGCCTGCCACGTTGCTGGATGGACCGTCG
>RXN01141-downstream
TAATCATCGGCATCACCGGCTTA
>RXN01142-upstream
CTCCCCATCCACCGGCACAGTCAGCGCAGGCAACGAAGAAATTAAAGGACCAGGACCTGA
CCGAGGCATGGTTTTCCAAGACCACGCCCTCCTGCCCTGA
>RXN01142
TTGACCGCACGCGGCAACATCGACTTCGGGCTCCGCTCCGCGCGCCCCTCCTTGAGCAAA
ACCGAACGCGCCGACATCACCCGCACCCACCTCGAACAAGTAGGCCTCACCGACGCCGCC
GAACGGCGCCCCGCCCGCCTCTGCGGCGGCATGCAACAGCGAGTCGGCATCGCACGCGCC
TTCGCCATCGACCCAGCAATCATGCTTCTCGACGAAGCCTTCGGCGCCCTCGAGGGCCTC
AGCCGCCGCGAACTCCAGCTCCAAGTACTCAACATTTGGGAAGCCTCCCGCCGGACCGTC
GTCATGGTCACCCACGACGTCGACGAGGCCATCCTGCTCTCCGACCGAGTTGTCGTGATG
TGCAAGAGCGCCGAAGCCACCATCATCACCGATATTCCAGTGAATCTTCCCCGCCCCAGA
CACGAGCTGAGTGAAGACGCTTCTGTTGAAGCCGAGACCACAGCGCTGCGTAAGCGGATG
CTGCATCTGCTGGAGCAC
>RXN01142-downstream
TAGTTTCTAACACGTCTTTTAAA
>RXN01164-upstream
GCCGATCGTGATTGATGAAGACGAGATCCAAGCCTGGACTTCTGATCTCAAACCTGAAGA
TTTCACCAAAGGTAAAGATGAATCCGACGGTGAGAAATAA
>RXN01164
GTGACACTGTTTGTTCGGCTCGCCCTTGCTGCTGTGGGCGGGCTTTTTGTGTTTGCTTCC
AATGAACCGATCGGCTGGTTTGTCGCGGGAATTGTTGGCACTGCATTATTTTTTATCTCC
CTTGCGCCGTGGGATGTGGGAGTTCGCCAAAAGCGGCGGAAGAAGAATGAGCCAGTCCCA
TTTTTGCAACAGATGTCCACGGGCCCAACTGTTGTACAGGGCATGCTTTTAGGTTTTGTC
CATGGCCTGGTGACATATTTGCAGCTGTTGCCGTGGATCGGTGAGTTTGTTGGCTCACTG
CCTTATGTCGCGTTGTCAGTTGTCGAGGCGCTTTATTCCATTGCTCTTGGTGCTTTCGGC
GTGCTCATTGCGCGTTGGAGGGACTGGAAGGTTCTCCTGTTTCGGGCGATGTATGTGGCT
GTGGAGTATCTAAGAAGCTCGTGGCCATTTGATGGATTCGCGTGGGTTGGCCTGGCATGG
GGTCAAATTAACGGTCCGTTGGCTAATGTCGCAGCGCTTGGTGGGGTAGCGTTTGTGACT
TTTTCCACGGTGCTGGCTGCCGTGGGTGTGGCCATGGTGATTATTTCCAAGAAGCGACTG
GCCGGCGCAATCATCACCGCGAGTGTGATTGCTATCGGGGCGGTGTCATCCCTGTACGTT
GACCGCAATGGCACGAGCGATGAAAGCATCGAAGTAGCCGCAATTCAGGGCAATGTGGCT
CGGATGGGATTGGACTTCAATGCACAGCGCCGCGCGGTGCTGGCGAATCACGCACGGGAA
ACCCTCAAGCTGGATGAACAAGTGGATTTGGTGATCTGGCCGGAGAATTCCTCAGACGTC
AACCCATTTTCCGATGCACAAGCAAGAGCCATTATCGATGGAGCAGTGGAACATGTTCAG
GCAGCTATTTTGGTGGGCACGATCACCGTCGATGAGGTTGGTCCACGCAACACCATGCAG
GTATTTGATCCTGTTGAAGGTGCCGCGGAGTACCACAATAAGAAGTTCTTGCAGCCGTTT
GGTGAATACATGCCGTTTCGCGAATTCCTGAGAATTTTCTCGCCCTACGTTGATTCCGCT
GGAAACTTCCAGCCCGGTGATGGCACCGGCGTAGTGGAGATGAATGCTGCGAACTTAGGC
CGCGCTGTGACAGTGGGCGTGATGACGTGTTACGAGGTCATCTTCGACCGTGCTGGCCGC
GACGCCATCGCCAATGGGGCTGAATTTTTGACCACGCCCACCAACAACGCCACCTTCGGA
TTCACGGACATGACGTATCAGCAATTAGCAATGAGCAGGATGCGTGCCATCGAATTTGAT
AGGGCGGTGGTTGTTGCAGCTACATCGGGTGTTTCGGCTATCGTCAACCGTGATGGAAGC
ATTTCCCAAAAGACCCGAATTTTTGAGGCCGCCACCTTGACGGAATCCATTCCACTCAAG
GACACTGTCACCATCGCAGCGCGGGTTGGTTTCTATGTTGAATTACTGTTGGTTATCATT
GGTGTATTAGCTGGACTATTCGCCATTCGAATGAATAGCCGTTCAAAGTCTGCGAAAGGT
TCCGCTCGGGCCGCACAAGTTCGGGTTAAGAAGGTGCCTGCGAAAAAGGGAGCAACTAAT
CGTCGAAAAGTAAAA
>RXN01164-downstream
TAAAAACGTCCCGAAGGGACGAG
>RXN01168-upstream
CCGCACAAGTTCGGGTTAAGAAGGTGCCTGCGAAAAAGGCAGCAACTAATCGTCGAAAAG
TAAAATAAAAACGTCCCGAAGGGACGAGGAGGACAACACC
>RXN01168
ATGAGCAGTGAGGCAGTAGATGCTACGACGCTGGTGATTATTCCAACGTACAACGAGCTG
GAAAACCTTCCACTCATCGTGGATCGCGTGCGCACCGCAACCCCTGACGTTCACGTACTC
ATCGTGGACGACAACAGCCCAGACGGCACCGGGGAGCGCGCAGACAAGCTTGCTGCTGAC
GACGACCACATTTTTGTCCTCCACCGCGAAGGCAAAGGCGGCCTGTGCGCAGAGTACATG
GCTGGCTTCCAGTGGGGCCTGGAGCGCGACTACCAGGTCCTGTGCGAAATGGACGCCGAC
GGCTCCCACGCACCAGAACAGCTGCACCTGCTGCTCGCTGAGATCACCAATGGCGCTGAC
GTGGTCATCGGCTCGCGCTACGTGCCAGGCGGCCGCGTAGTCAACTGGCCCAAGAACCGT
TGGCTCTTGTCCAAGGGCGGCAACGTCTACATGAGCGTCGCGCTCGGCGCCGGCTTGACC
GATATGACCGCAGGGTACCGCGCTTTTGGACGTGAAGTGCTAGAAGCACTGCCGGTTGAT
GAGCTCTCCAACGCTGGGTACATTTTCCAAGTTGAGATTGCCTACCGTGCAGTTGAAGCC
GGATTCGATGTTCGTGAAGTTCCCATCACTTTCACCGAGCGTGAGATCGGCGAATCCAAG
CTGGACGGCAGCTTTGTCAAGGATTCCCTGGTCGAGGTAACCAAGTGGGGCCTCAAGCAC
CGCGGTGGCCAGGCCAAGGAACTGTCCAAGGAAATGGTGGGGCTGCTGAACTATGAGTGG
AAGCACTTCAAAAAGCGCAACACCTGGCTC
>RXN01168-downstream
TAAACTGCTTGCCGGTTAGTGAA
>RXN01285
CTCAACGTCACCATCCCCGACAACAGCTTCACCGCCATCATCGGCCCCAACGGCTGCGGC
AAATGCACGCTGCTCCGCGGTTTCTCCCGCGTGCTCAATCCGCAGCACGGCAAAGTGCTT
CTCGACGGTCGGCAACTCGATTGATTCAAGCCTAAAGAGATCGCCCGAGAACTAGGCCTG
CTGCCACAGACCTCCATCGCCCCAGAAGGCATGCGGGTTTACGATGTCATCGCGCGCGGG
CGCGCTGGCTACCAAAGCCTCATACAACAATGGCGCACCTCCGACGAAGACGCCGTCGCG
CAAGCGCTCGCCTCCACGAATCTGACCGAACTTGCAGCTCGCCTCGTCGATGAACTCTCC
GGTGGCCAGCGCCAACGAGTGTGGGTGGCCATGTTGCTCGCCCAGCAAACACCGATCATG
CTTCTCGACGAGCCCAGCACCTTCCTCGACATGGGCCACCAATACGAACTCTTGGAATTG
CTGCGCGCATTCAACGAGGCCGGGAAAACTGTGGTCAGTGTGCTTCACGATCTCAACCAA
GCCGCCCGCTACGCCGACCACCTCATCGTGATGAAAGATGGGCACGTACATGCCACGGGC
ACACCGGAGGAAGTCTTAACTGCCGAGATGGTTCAAGGAGTTTTTGGCCTGCCCTGCATC
ATCTCCCCAGACCCCGTCACAGGAACCCCGACCGTCGTTCCCCTCAGTCGGTCTCGCGCA
GGAGCT
>RXN01285-downstream
TAAGTAGGTACCCCTCCAACGGA
>RXN01298-upstream
CTTAAACGTCACCTTATTTATGCATTATGTTGGTTTCAGACTCGAACAATTCAATTAGAA
AACACTAATCGGACATTTAGGTCACATAACATTTCCGCTC
>RXN01298
GTGTCCACATTAATTTCTGAACCCGAGGTGGATAAGCTACGTAAACGTGCCAAGAGATCA
AGGCGGACAGAATGGTGGCTTGCCGCCGCACTTCTTGCCCCAAACTTGCTTCTCTTGGCC
ATCTTTACGTATCGGCCACTGTTAGATAACTTCCGGTTGTCCTTTTTCAACTGGAACATT
TCCTCGCCCACATCAACCTTCATTGGGTTTGATAACTACGTTGAGTTCTTCACTCGTAGT
GACACTCTCCAAGTTGTTTTAAACACCGTCATCTTCACGGCATGTGCTGTGATCGGATCG
ATGGTGCTCGGTTTGCTCCTGGCCATGTTGTTGGATCAGAAGCTTTTCGGCCGTAACTTT
GTGCGTTCGATGGTGTTTGCCCCGTTTGTGATTTCCGGTGCTGCCATTGGTGTTGCTTTC
GAGTTGGTTTTTGACCCTAATTTTGGTTTGGTTCAGGACTTGCTGGGACGCATCGGCGTT
GATTCGCCACAGTTCTACCAAAACCCTAACTGGGCATTGTTCATGGTGACGTTCACTTTC
GTGTGGAAGAACTTGGGCTACTCCTTTGTTATCTACCTGGCTGCATTGCAGGGGCTAAAC
AAGGATTTGTCTGAGGCCGCACCGGTGGATGGCGCGAGCGCGTGGACACGTTTTTGGAAG
GTTACTCTTCCGCAGCTTCGCCCAACCACGTTCTTCCTTTGTATTACTGTCACGCTGAAC
TGGGTTCAGGTCTTCGACATCATTCACACCATGACTCGTGGTGGCCCCTTGGGTAACGGT
ACGACCACCTTGGTTTACCAGGTGTACACCGAGACTTTCACCAACTATGGCGCGGGATAT
GGTGCAACAATCGCAACGATTTTGTTCCTGTTGCTGCTGATTATCACTGTTATCCAGGTT
CGATACATGGATAAGGAGAACAAGCAGAAA
>RXN01298-downstream
TGATCTCGACTGATAGAAACGTT
>RXN01323-upstream
CACGTGGTTTACGCCAGGCATGTTCCCGCGAAGGGTTGACCCATACCCCTAGGGGGTATA
CAGTGAGTCATGTAAACATACTCGCAGAAGGAGCGATCCC
>RXN01323
ATGGCTCAGACACCCGCCAAAATCCCGGCGGCACTGAATTTCATTGACGTCGACCTCGGC
GTTACCGGCATGACCTGCACTTCTTGCTCCGCCCGCGTCGAGCGCAAACTGAACAAGCTC
GACGGCGTTGAAGCAACCGTCAACTACGCGACGGAATCCGCACAGGTCAGCTACGACGCG
TCAAAGGTCAGCCCTGAACAGCTGATTAAGACTGTTGAGGACACCGGCTACGGTGCTTTC
ACGATGGCTTCCGCAGCTGCCGAATCAGAAGAGGACAACGCTCCAGGTGACAGGGGCGAG
TGCCGCATCGACGCAGCTCGCGAGCACGAAGCAGGCGACGTGAAACACCGCGTGATCGTC
TCTGCACTGTTGTCAGTTCCTGTGGTTTTGGTCAGCATGATCCCGGGGCTGCAATTCAAC
AACTGGCAGTGGGCCGTACTCAGTTTGGTCACCCCGATTTTCTTGTGGGGCGGTTCACCG
TTCCACAAGGCAACGTGGGCAAACCTGAAGCGCGGTTCCTTCACCATGAAGAGCCTGGTT
TCACTCGGCACGTCCGCTGCTGACCTGTGGTCCGTGTGGGCTTTGTTCATTGAAAATGCT
GGTCACCGTGGCATGAAGATGGAGATGCACCTGCTGCCGTCGGCCTGCACGATGGATGAG
ATTTACCTGGAAACCGTCGCGGTCGTTATTACGTTCCTGCTGCTTGGACGCTGGTTTGAG
ACAAAAGCTAAGGGCCAATGTTCGGAAGCTCTGCGCAAGCTGCTGGACATGGGCGCCAAA
GATGCAGTCGTGTTACGTGACGGCGCCGAAGTCCGCGTTCCTGTGAATCAGCTTAAACTC
GGCGACGTTTTCATCACCCGCCCCGGCGAGAAAATCGCCACCGACGGTGAAGTCGACGAA
GGTTCCTCCGCAGTCGACGAATCCATGCTCACCGGCGAATCCATCCCCGTTGAAGTCACC
AAGGGGTCCAAAGTTACCGGCGCAACGCTGAACAGTTCCGGCCGCCTCATGGTGAAAGTA
ACCCGCATCGGCGCCGACACCACCCTGTCGCAAATGGCTAAACTGGTCACGGACGCACAG
TCCAAAAAGGCCCCTGTCCAGCGTCTTGTTGACCAAATCTCGCAGGTTTTCGTTCCCGTT
GTCATCGTAATTGCTATTGCGACGCTGATCGCGCACGTCGTCTTCACCGACGCCGGCCTC
GCCCCAGCATTCACGGCAGCAGTCGCCGTCCTCATTATCGCCTGCCCTTGTGCCCTCGGC
CTGGCAACCCCAACCGCACTTCTGGTCGGAACGGGCCGCGGCGCGCAACTTGGTCTGTTG
ATCAAGGGCCCTGAAATCCTCGAATCCACCAAAAAAGTCGACACCATCGTCCTCGACAAA
ACCGGCACCGTCACCACCGGCACCATGTCCGTCACCGACGTCACCGCCATCAACTACAGC
GAAACCGAAATCCTCGAATTCGCTGCAGCCGTCGAGTCCGCCTCCGAACACCCCATCGCC
CAGGCAATCGCCAAGGCCGCCGAACACGAGCAAGTCACCGACTTCCAAAACACCGCAGGT
CAGGAAGTCACCGGTGTAGTCCGCGGACACGAGGTCCGCGTGGGCAGGCCTTCAAGCACG
GTTATCGACGCCCTCCTCCACCCCTTCCAACACGCCCAAAAAATCGGCGGAACCCCCGTA
GTCGTCACGATTGACGGCGTAGATTCCGGAATAATCACGGTCCGCGACACCGTCAAAGAC
ACCTCCGCCGAAGCAATCCGCGGACTCAAGGAACTGGGACTCACCCCAATCCTACTCACC
GGAGACAATGAAGGCGCAGCTAAATCCGTAGCCGCTGAAGTCGGCATCGACCAAGTCATC
GCCAACGTCCTCCCCCACGAAAAAGTCCAAAACGTAGAAGCCCTCCAAGCACAAGGCAAA
AACGTTGCGATGGTCGGCGACGGCGTCAACGATGCCGCAGCTCTTGCCCAAGCTGACCTC
GGACTCGCCATGGGAGCCGGCACCGACGTAGCCATCGAAGCCTCCGACATCACCCTCATG
AACAACGACCTCCGATCCGCAGTCGACGCCATCCGACTGTCCCGTAAAACCCTCGGCACC
ATCAAGGGAAACCTTTTCTGGGCTTTCGCCTACAATGTTGCACTAATCCCAGTAGCGGCG
ATCGGACTCCTCAACCCAATGCTTGCCGGCATTGCGATGGCCTTCAGTTCAGTTTTCGTC
GTCTCCAATTCCTTGCGTCTGCGAGGATTCAAAGCAAGGAGCAAC
>RXN01323-downstream
TAATGTCCAACAGCGAATGCCAC
>RXN01338
AAAACTTATACCCCAAATGCCTGGATGTTATTCATCCGCTCATTTGATGGCATCATCACT
GTCGCAGCCCTTGTTGCCATCGCAATACATCTCATTTTATGGCTGGCTCTAGATCTAGAT
GGCGTTGCTAAAAACTGGCCTTTAATAGCCATCGTTATCGTAGGTGGCATTGCGTTGATG
TGGGATGTGCTGAAATCAGCCATTAAAACTCGCGGTGGCGCGGATACTTTAGCAGCAGTC
TCCATCATTACTTCTGTGTTGTTAGGGGAGTGGTTGGTTGCCGCGATCATCGTGCTCATG
CTCTCTGGTGGTGAAGCGCTAGAAGAGGCAGCATCACGGCGAGCGAGTGGCACCTTGGAC
GCACTTGCCCGGCGCGCACCAAGTACAGCTCACCGCCTGTTGGGTGGAACCATTCTTGAT
GGAACCGAAGAGATCGCCGTGGAAGAGATCACGGTTGGTGATTTAGTGGCGGTGCTCCCG
CATGAACTTTGTCCCGTGGATGGTGAAATCGTGGCAGGCCACGGCACCATGGATGAGTCT
TATCTCACGGGTGAGCCCTATGTGGTGAGTAAATCTAAAGGTTCGCAAGCAATGTCGGGT
GCAGTCAATGGTGATACTCCGCTGACGATTGTTGCCACAAAGCTTGCCCATGATTCCAGA
TACGCCCAAATTGTTGGTGTACTCCATGAAGCAGAAAACAACCGCCCAGAAATGCGCAGG
ATGGCTGACCGTCTTGGCGCGTGGTATACGGTGATTGCACTTGCCCTCGGTGGTCTTGGC
TGGATTGTCTCCGGCGACCCAGTGAGGTTCTTGGCTGTTGTCGTTGTCGCCACCCCATGT
CCATTGCTCATTGCAGTGCCAGTGGCGATCATCGGTGCGATTTCTCTTGCGGCTCGTCGG
GGCATCATCGTGAAGAACCCTGGAATGCTGGAAAACGCTTCAGGAGTAAAGACAGTGATG
TTCGATAAGACTGGAACGCTCACCTATGGCAGGCCAGTGATTACTGATATCCACACTGCT
CCCGGAGTTGAGGAAGATACAGTCCTAGCTTTGGCTGCTTCAGTAGAGCGCTACTCCAGA
CACCCGTTGGCTGACGCGATTCGTGAGGGCGCAAAAGCCAGGGAACTTCATCTGCCTGAT
GTAGTGGAAGTATCGGAACGTCCAGGACAGGGACTAACCGGCACGGTGGGCGAGCACCTG
GTTCGAATAACCAATAGGCGCAGCACACTAGAAATTGATCCAGACAGCAAGAACTACATT
CCGGTGACAAGTTCCGGCATGGAATCTGTGGTGCTTGTTGATGATAAATATGCAGCACTC
ATTCGCGTCCGGGATGAACCTCGTGCATCTGCGAGTGAGTTCATCGCGCACTTGCCCAAG
AAGCACAAAGTGGACAAGCTCATGATTATCTCTGGTGATCGCGCATCTGAGGTTCGTTAC
CTTGCGGACAAGGTTGGCATTGATGAGGTACACGCAGAGGCCTCACCGGAAGACAAGCTG
AACATTGTTAATCGGCATAATGAGCACGGCGCCACCATGTTCTTAGGTGATGGAATCAAC
GATGCGCCAGCGATGGCCGTTGCCACCGTTGGTGTCGCGATGGGAGCAGACTCCGATGTC
ACGTCCGAAGCAGCAGATGCTGTGATTTTGGATTCTTCCCTGGAACGTCTCGACGATCTG
CTCCACATCAGTGCACGGATGCGTCGAATAGCGTTGCAATCTGCGGGCGGTGGCATGGCG
TTGAGTGTCATAGGAATGATCCTCGCGGTATTTGGATTCTTGACGCCACTGATGGGTGCG
ATCTTCCAAGAGGTCATTGACGTGCTGGCTATCCTCAATTCCGCTCGGGTCGCACTGCCA
CGCGGAGCGATTAGTGATTTTGATACGCAAGAAAAAGTTTCT
>RXN01338-downstream
TAGCAGGGTAACCTAAATGTCGT
>RXN01411-upstream
CTTATCGACGTCCCCATCCCCCTCGCCAATGCTTCGGCGAGGGGTTCTATTTATTGTGTG
TGCTAGCCTTTTCGCAATCGTTCAGCCCGCCCCGACGTCA
>RXN01411
ATGTTGGGAGTGGGCTGGCGCATTCCATTCCTGATGGCCGTGCCACTAGGGCTTATCGGC
TGGTGGATGGGCACCGGTGCCCAGGAAAATGTACGCCCCGCATCCGAACGCCCCGAAGCT
CCTATTAAGCAGGCATTGCGTACTGAGTGGAAGATGATGTTGCGGGTAGGTGGCTTTATC
TCTTGCACCGGTCTGAGCTTCTACATTTTCACCACGTACATGACCACTTTCCTGCGCAGC
ACCGTCGGACTGGAGGGCACGTTAGTGCTGGCTGGAAACATCATCGCTCTGAGCATGGCA
GCAATTGTGGCCCCATTTGTTGGCCGCGCAATTGATAAATTCCCCCGCCGGAACATCATG
GGTTTCGCTACCTTAAGCACAGTAATTATGGCGATCCCGGCCTACATCATTGCAGGTCAA
GGTACTTTGACTGCTTCTTTGATTGCGCAGGTAATGCTTGGAATCGGCGCGGTTACCGCT
AACTGCGTTACCTCAGTAATGATGGCCGAGGTCTTCGAAGAGGTCACCCGCGGTACTTCC
GCCGGCATTACCTACAACGTCACTTACGCAATCTTCGGCGGCTCGGCTCCATTTATCTCC
ACCGCATTGGTCTCCTGGACCGGCAGCCCGCTGGCCCCTGCGGTATACATGATCATCATT
GGGCTCTTCGCCTTCACCGCGTCCCGCTTCATTCCTGAAACCTCCCCAGTTTTTGTCACC
GCAACCCCGGCCATTAAGGCACCAAAGGTGCTGGTCAACCCGGGT
>RXN01411-downstream
TAAACCACGCTTTTCGACGAAAA
>RXN01808
CAGAGCCTCGCGTGTAAAGAACTCGCATGGATGCGGGGCGGTGCACCAGCGCGAACCTCA
AAGCCTGGATTCCGCCTTGAAGCCGCGGAAGCTTTGATCGCAGAAGTGCCAGCGCCACGC
GACAAAGTCGAGCTCATGGCATTTTCCAAGTCCAGGCAAGGCCGCGTTGTCATTGAACTT
GAAGACGCGACAGTAGCCAGCCCTGATGATCGCATCCTGGTAGAAGAGCTGACCTGGCGT
TTGGCTCCAGGAGAGGGCATCGGTCTTGTCGGCGTCAACGGGTCCGGCAAAACCACCCTG
CTGCGCACCCTTGCCGGCGAGCAGCCACTTCAGGCAGGCAAACGCATCGAAGGCCAAACC
GTCAAACTGGGATGGCTCCGCCAGGAACTCGATGACCTAGACGTCAGCCGCCGACTCATC
GACTGCGTTGAAGATGTCGCTTCCTACGTGATGATGGGCGACAAGCAGGTCTCCGCTTCC
CAATTGGCAGAACGCCTCGGATTCTCACCCAAGAGGCAACGCACCCCAGTTGGTGACCTG
TCCGGTGGTGAACGCCGCCGACTCCAACTCACCCGCGTGCTCATGGCCGAACCAAACGTG
CTGCTCCTCGACGAGCCCACCAACGACCTGGACATTGACACCCTCCAAGAGCTGGAATCC
CTTGTCGACGGATGGCCAGGCACCATGGTGGTTATCTCCCACGACCGTTACCTCATCGAA
CGCGTCACCGACTCCACCTGGGCACTCTTCGGCGATGGCAAGCTCACCAACCTGCCAGGC
GGAATTGAAGAGTACCTGCAGCGACGAGCAGCGATGGCCGCGGCCGAAGACAGTGGAGTG
CTGAACTTGGGTGCGGCCACGCAGGCTGGAACCTTTTCTGCTGCAACAGAGCAGGCTGCC
ACTTCTGTGGAAAGTTCCGGAATTTCTTCCCAAGAACGCCACCGCATCACCAAGGAAATG
AACGCCCTGGACCGCAAAATGGGCAAGCTTGACCAGCAAATGGACAAGCTTAATCAGCAG
CTCGCTGATGCAGCGGAGGCCATGGACACCATAAAGCTCACCGAGCTGGACACCAAGCTC
GGCGCAGTGCAGGAAGAACACGGCGAGCTGGAAATGCAGTGGCTGGAACTCGGCGAGGAA
ATCGAGGGC
>RXN01808-downstream
TAGTTCATGCCGTCGGCAGGCGA
>RXN01939-upstream
TGCTGTTCTACCCCGCAATGGCACTTGCACTAACCGTTTTGAGCTTCATCATGATGGGCG
ATGTCGTCCGCGACGCTCTGGATCCTAAGTCGAGGAAGCG
>RXN01939
ATGACCACCAACATCCCACAAACCCCCAACCACGAGGGTGAACAGCCACTGCTCGAGCTG
AAGGATCTAAAGATTTCCTTCACCTGCTCCACCGGTGTTGTCGACGCTGTCCGTGGCGCA
AACCTCACGATTTATCCTGGCCAATCTGTTGCCATCGTGGGTGAATCCGGTTCAGGTAAA
TCGACCAGGGCAATGTCGATCATCGGTCTGCTTCCAGGCACCGGCAAAGTGACCGAAGGT
TCCATCATGTTTGATGGCCAAGACATCACAGGCTTGAGTAACAAGCAGATGGAAAAGTAC
CGCGGTTGAGAAATCGGACTGGTGCGCCAGGATCCGATGACCAACTTGAACCCGGTGTGG
CGCATGGGCACCCAGGTCAAGGAATCCCTCCGAGCCAACCACGTGGTTCCAGGCTCAGAG
ATGGACAAGCGCGTGGCAGAAGTTCTGGCCGAGGCAGGTCTTCCTGATGCTGAGCGTCGC
GCAAAGCAGTACCCACATGAGTTCTCTGGCGGTATGCGCCAGCGCGCACTGATCGCCATT
GGTTTGGGGGCACGCCCGAAGCTCTTGATCGCCGACGAGCCCACCTCTGCGCTGGATGTC
ACCGTGCAGCGCCAAATCCTTGATCACCTTGAAACACTGACCAAGGATCTCGGCACCGCA
GTGCTATTTATTACCCACGACTTGGGGCTTGCCGCTGAGCGCGCGGAGCACCTCGTGGTG
ATGCACCGCGGACGCATCGTGGAGTCCGGGCCATCATTGAAGATTCTGCGCAATCCACAG
CACCCATATACCCAACGCTTGGTTAAGGCTGCGCCGTCTCTGGCTTCTGCACGTATTCAA
AGTGCGCAGGAACAAGGCATTGAATCTGCAGAACTGCTCTCTGCAACGGCCGTTGCTGAG
GGCACTATTCCAGAGATGGAAGAAAAAGTTATCGAGGTGAAAAACCTCACCCGCGAATTT
GATATCCGCGGTGCCCGTGGCGATAAGAAGAAGCTGAAGGCCGTTGATGATGTGTCCTTC
TTCGTACGTAAAGGCACCACCACCGCACTTGTGGGTGAATCCCGTTCGGGTAAATCCACC
GTGGCCAACATGGTGCTCkACCTTCTCGAGCCAACCAGCGGAGAGGTGCTCTACAACGGC
ACCGATCTTACGTCCTTGAGCCACAAGGAAATCTTCCAAATGCGACGCAAACTGCAGGTG
GTGTTCCAGAACCCCTACGGCTCGCTTGATCCGATGTACTCCATCTACCGGTGTATTGAG
GAACCGCTGACCATCCACAAGGTTGGTGGAGACCGCAAGGCACGCGAAGCTCGCGTCGCT
GAACTTGTCGATATGGTGTCCATGCCCAGGTCCACCATGCGCCGCTACCCCAACGAGCTT
TCCGGTGGCCAACGTCAGCGCATCGCCATCGCCCGTGCATTGGCACTGAATCCAGAAGTG
ATCGTGTTGGATGAAGCGGTTTCCGCTTTGGACGTGTTGGTTCAGAACCAGATCCTCACC
CTGCTTGCAGAACTTCAGCAGGAACTGAAGCTCACCTATTTGTTCATCACCCACGACTTG
GCCGTTGTTCGACAAACCGCCGACGATGTTGTGGTGATGCAAAAGGGACGAATCGTTGAA
AAGGGTCGTACCGACGACATCTTCAACGATCCTCAGCAGCACTACACCCGCGATTTGATC
AATGCGGTACCTGGTCTGGGAATCGAGTTGGGTACTGGAGAAAACCTGGTT
>RXN01939-downstream
TAACCCGCACAGCCTCACTAAAC
>RXN01995-upstream
CCGACGCAAAGGCATGCGCCTGCGTGTCTCGAGTAGTCTCCTCCCCTTCCTCGTCCCCAA
CCTCGACCATTACGGTCGCGCTCTCCTAAAGGAGCCTGGC
>RXN01995
ATGGATATCCGCCAAACAATTAAGGACACAGGAATGTCGAGATATCAGTGGTTCATTGTA
TTTATCGCAGTGCTGCTCAACGCACTGGAGGGCTTTGATGTCCTCGCCATGTCTTTTAGT
GCGAATGCAGTGACCGAAGAATTTGGACTGAGTGGCAGCCAGCTTGGTGTGCTGCTGAGT
TCCGCGCTGTTCGGCATGACCGCTGGATCTTTGCTGTTCGGTCCGATCGGTGACCGTTTC
GGCCGTAAGAATGCCCTGATGATCGCGCTGCTGTTCAACGTGGTGGGATTGGTATTGTCC
GCCACCGCGCAGTCCGCAGGCCAGTTGGGCGTGTGGCGTTTGATCACTGGTATCGGCATC
GGCGGAATCCTCGCCTGCATCACAGTGGTGATCAGTGAGTTCTCCAACAACAAAAACCGC
GGCATGGCCATGTCCATCTACGCTGCTGGTTACGGCATCGGCGCGTCCTTGGGCGGTTTC
GGCGCAGCGCAGCTCATCCCAACATTTGGATGGCGCTCCGTGTTCGCAGCCGGTGCGATC
GCAACTGGTATCGCCACCATCGCTACTTTCTTCTTCCTGCCAGAATCCGTTGATTGGCTG
AGCACTCGCCGCCCTGCGGGCGCTCGCGACAAGATGAATTACATTGCGCGCCGCCTGGGC
AAAGTCGGTACCTTTGAGCTTCCAGGCGAACAAAGCTTGTCGACGAAAAAAGCCGGTCTC
CAATCGTATGCAGTGCTCGTTAACAAAGAGAACCGTGGAACCAGCATCAAGCTGTGGGTT
GCGTTCGGCATCGTGATGTTCGGCTTCTACTTCGCCAACACTTGGACCCCGAAGCTGCTC
GTGGAAACCGGAATGTCAGAACAGCAGGGCATCATCGGTGGTTTGATGTTGTCCATGGGT
GGAGCATTCGGCTCCCTGCTCTACGGTTTGGTCACCACCAAGTTCAGCTCCCGAAACACA
CTGATGAGCTTCATGGTGCTGTCCGGGCTGACGCTGATCCTGTTCATTTGCTCCACCTCT
GTTCCATCCATCGCGTTTGCCAGCGGCGTTGTCGTGGGCATGCTGATCAATGGTTGTGTG
GCTGGTGTGTACACCCTGTCCCCACAGCTGTACTCGGCTGAAGTACGCACCACTGGTGTG
GGCGCTGCGATTGGTATGGGTGGTGTGGGTGCGATTTCCGCGCCACTGCTGGTGGGTGGC
CTGCTGGATTCTGGCTGGTCCCCAACGCAGCTGTATGTTGGTGTGGCAGTGATTGTTATT
GCCGGTGCAACCGCATTGATTGGGATGCGCACTCAGGCGGTAGCCGTCGAAAAGCAGCCT
GAAGCCCTAGGGACCAAA
>RXN01995-downstream
TAGGGCCGCGATTCCTAGCATGC
>RXN02062-upstream
TTGTCTAAACATCGTTTTGGGGTGCGAATGATAGCCCCTTTTAATGCCCCCATTTCGGTA
TCGCTGCGCAACTGTTTTTAGATGGCTAATCTTTGAAATT
>RXN02062
ATGAGAGTCGGAATGATGACAAGAGAGTATCCACCAGAGGTTTACGGCGGCGCTGGCGTG
CACGTCACCGAATTGACCCGATTCATGCGTGAGATCGCTGAAGTTGATGTTCACTGCATG
GGTGCAGCTCGCGATATGGAGGGAGTTTTCGTCCACGGCGTCGATGCTGCCTTGGAAAGC
GCGAACCCTGCGATTAAGACACTGTCCACCGGTTTACGCATGGCAGAAGCTGGAAACAAC
GTGGATGTCGTGCACTCACACACTTGGTATGCAGGTCTTGGCGGCCACCTTGCAGCTCGT
CTCCACGGCATTCCTCACGTGGCTACCGCGCACTCTTTGGAGCCAGATCGCCCATGGAAG
CGTGAGCAGCTTGGCGGTGGATACGACGTGTCCTCCTGGTCTGAAAAAAATGCCATGGAA
TACGCTGACGCGGTCATCGCTGTGTCGGCTCGCATGAAAGATTCCATCCTCGCTGCGTAC
GCTCGCATCGAGCCGGACAACGTGCGTGTTGTGCTCAACGGCATCGACACTGAGTTGTGG
CAGCCTCGCCCGACTTTCGATGACGCGGAAGATTCCGTACTCCGCTCCCTAGGCGTTGAC
CCACAGCGGCCCATCGTCGCATTTGTCGGCCGCATCACCCGCCAAAAAGGCGTCGAGCAC
GTCATCAAGGCAGCAGCGCTTTTCGACGAGTCCGTGCAGCTTGTGCTCTGTGCCGGCGCG
CCAGACACCCCCGAAATCGCAGCTCGCACCACCGCCCTGGTGGAAGAACTCCAGGCAAAG
CGCGAAGGCATTTTCTGGGTTCAGGACATGCTGGGCAAGGACAAAATCCAAGAGATTCTC
ACCGCTGCTGACACCTTCGTGTGCCCATCCATTTACGAGCCACTGGGCATCGTGAACTTG
GAAGCAATGGCCTGCAACACCGCAGTTGTCGCATCCGACGTTGGAGGCATCCCTGAGGTT
GTTGTCGACGGCACCACCGGCGCCCTCGTTCACTACGACGAAAATGATGTCGAAACCTTC
GAGCGCGATATCGCCGAAGCGGTGAATAAAATGGTCGCTGATCGAGAGACCGCAGCCAAA
TTTGGTCTCGCAGGGCGCGAACGTGCTATCAATGATTTCTCCTGGGCAACGATTGCTCAG
CAGACCATTGATGTGTACAAATCCTTGATG
>RXN02062-downstream
TAAAACCGAAAGCCGGGGAACCT
>RXN02096-upstream
CGCTTCGACGACCTCACCCACAGCGATATCCGCAGGAATGTCATCGCGGTTTTTGATGAG
CCGTTCTTGTACTCCTCCTCCATACCGGGAGAACATCTCG
>RXN02096
ATGGGTTTGGATGTCAGTGATGAGGAGATCGAACACGCAGCCAGGCTTGCCCAGGCTCAT
GATTTTATCGATCGCCTTCCAAACAAATACGAGGAAGTCATTGGCGAACGCGGCCTGACG
CTTTCTGGTGGTCAACGCCAACGCATCGCCCTCGCACGGGCTTTCCTGGCGCATCCCAAA
GTGTTGGTGCTTGATGATGCCACCTCTGCCATTGATGCCTCCACTGAGGACCGCATTTTC
CAGGCCTTGCGCGAAGAACTGCACGATGTCACCATTTTGATCATCGCGCACCGCCACTCC
ACTTTGGAGCTCGGCGATCGGGTTGGTCTGGTCGAAGATGGACGGGTAACAGCACTGGGA
CCGTTGAGTGAGATGCGTGATCACGCTCGTTTCTCGCATCTGATGGCTCTTGATTTCCAG
GATTCTCACGATCCGGAATTCACCCTCGACAACGGTTCACTACCCAGCCAAGAGCAATTG
TGGCCGGAGGTCTCCACAGAAAAGCAGTACAAGATTCTTGCGCCTGCCCCTGGTCGAGGC
CGTGGCATGTCCATGCCAGCAACCCCTGAGCTGCTCGCCCAGATTGAGGCGCTGCCAGCA
GCAACGGAAGAAACACGAGTTGATGCCGGGAGGCTACGCACCAGTACCTCCGGTTTCAAA
TTGCTCAGTTTATTCAAGCAGGTCCGTTGGCTCGTCGTCGCGGTCATCGCGTTGTTGCTG
GTGGGCGTAGCCGCCGATCTAGCATTTCCAACACTGATGCGCGCAGCCATCGACAACGGT
GTGCAAGCACAAAGCACCTCCACGTTGTGGTGGATCGCCATCGCAGGCAGCGTAGTAGTC
CTTCTGTCCTGGGCCGCCGCCGCGATCAACACGATTATCACGGCACGCACCGGTGAACGG
GTGCTTTACGGCTTGCGTCTGCGCTCATTTGTGCATCTATTGCGCCTGTCCATGAGCTAT
TTCGAACGCACCATGTCCGGCCGCATCATGACGCGCATGACCACCGACATCGACAACCTC
TCGTCCTTCCTCCAATCAGGTCTGGCGCAAACAGTTGTCTCTGTGGGCACGCTCATCGGT
GTGGTCACCATGCTCGGCATCACCGACGCACAACTAGCACTCGTTGCGCTGTCCGTGGTG
CCGATCATCATCGTGCTCACTCTCATTTTGGGACGCATCAGCTCCAGGCTGTACACCGCT
TCACGGGAGCAAGCCAGCCAGGTCAACGCGGTATTCCACGAGTCCATCGCCGGTTTACGC
ACCGCGCAGATGCACCGCATGGAAGACCAAGTCTTTGACAATTATGCGGGCGAAGCAGAG
GAATTCCGACGCCTGCGTGTGAAATCCGAGACGGCCATCGCCATCTACTTCCCCGGCCTT
GGGGCGCTCTCTGAAATCGCCCAGGCACTCGTCCTCGGTTTCGGCGCACTGGAAGTAACG
CGGGGGGACATCTCCACCGGGGTACTCGTGGCATTCGTGCTGTACATGGGCCTGATGTTC
GGCCCGATCCAACAACTAAGGCAAATCTTCGACTGCTACCAACAAGCCGGCGTCGGCTTC
CGTCGCATCACCGAACTGCTGGCAACGGAGCCCAGCGTCCAGATCTGGGCACCAACAGGC
ACGCTAGGCAGGCTGCCACGCAGCCTTTATTGCTTGACGACGTCACCTTCGGCTATTCAG
ACGATCCGATCC
>RXN02096-downstream
TAGACAACGTCACCGTCCAGATC
>RXN02348-upstream
AAAGACCCGAGCCGAAGCCCTGGCCTGCGCATACTTCCTTGTCAACGCTCGCTGGGATTA
GGTCTTTTCTGAGCGCTAGCATTTCTCCACTCAAAGGAGC
>RXN02348
ATGCTTAACCGCATGAAAAGTGCGCGGCCAAAATCAGTCGCTCCAAAATCCGGACAAGCT
TTACTCACTCTCGGTGCCCTAGGTGTTGTGTTCGGCGACATCGGCACCAGCCCCCTGTAC
TCACTTCACACTGCATTCAGCATGCAGCACAACAAAGTCGAAGTCACTCAGGAAAATGTG
TACGGCATCATCTCGATGGTGTTGTGGACCATCACTTTGATCGTCACCGTCAAATACGTC
ATGCTGGTCACCCGAGCTGACAACCAAGGACAAGGTGGCATCCTGGCGCTCGTTGCTTTG
CTGAAAAACCGTGGGCACTGGGGAAAATTCGTGGCAGTAGCCGGCATGTTGGGCGCCGCA
TTGTTTTATGGCGATGTGGTGATCACCCCGGCGATCTCTGTTCTCAGCGCAACAGAAGGC
TTGACGGTTATCTCCCCAAGCTTTGAGCGCTTCATTCTGCCCGTATCTCTGGCAGTTCTG
ATCGCTATTTTTGCAATCCAACCGCTCGGTACAGAAAAAGTCGGCAAAGCCTTCGGCCCC
ATCATGTTGCTGTGGTTTGTCACCCTTGCAGGATTGGGAATTCCGCAAATCATCGGGCAC
CCAGAAATCTTGCAGAGCTTGTCTCCACATTGGGCCCTGCGCTTGATTGTGGCTGAGCCT
TTCCAAGCATTTGTGCTGCTTGGTGCCGTTGTCCTGACAGTAACGGGTGCGGAAGCGCTC
TACGCTGATATGGGCCATTTTGGGGCGAGGCCAATCAGAGTGGCGTGGTTTTGCGTCGTC
ATGCCTGCTTTAATCTTGACGTATTTGGGGCAGGGCGCCTTGGTGATCAACCAGCCTGAA
GCGGTGCGCAACCCCATGTTTTATCTCGCGCCGGAAGGTCTGCGGATTCCGTTGGTTATT
TTGGCGACCATCGCTACGGTGATCGCATCGCAGGCCGTGATTTCTGGTGCGTATTCATTG
AGCAAGCAGGCCGTGAATTTGAAACTGCTGCCACGCATGGTGATCCGGCATACCTCCCGC
AAAGAGGAAGGCCAGATCTATATGCCACTGGTTAATGGATTGCTGTTTGTATGCGTGATG
GTTGTGGTGCTGGTATTCCGATGCTCTGAAAGGCTCGGCAGCGCGTACGGACTTGCAGTG
ACCGGAACCTTGGTGCTGGTCAGCGTCCTGTATCTGATCTATGTTCACACCACATGGTGG
AAAACAGCGCTGTTCATTGTGCTCATCGGTATTCCAGAAGTACTTCTATTCGCCTCGAAC
ACCACGAAAATTCACGACGGTGGCTGGCTTGCACTACTTATTGCGGCCGTGCTGATCGTG
GTGATGCGGACCTGGGAGTGGGGAAGTGACCGCGTCAATCAGGAACGCGCAGAGCTGGAA
CTTCCCATGGATAAGTTCTTGGAGAAACTCGATCAGCCACACAATATTGGTCTGCGTAAA
GTTGCCGAAGTGGCAGTATTTCCACATGGGACGAGCGATACTGTCCCGTTGTCATTGGTT
CGCTGCGTGAAAGACCTCAAGCTTTTATACCGAGAGATCGTGATCGTTCGAATCGTCCAA
GAACACGTTCCGCACGTGCCACCAGAGGAACGCGCGGAAATGGAAGTGCTCCATCACGCC
CCGATCAGAGTCGTGCGAGTTGATCTGCACCTTGGTTATTTTGATGAGCAGAACCTGCCT
GAGCATCTCCATGCCATTGACCCAACATGGGATAACGCCACCTACTTCCTGTCTGCCCTG
ACTCTTCGGAGCAGGTTGCCTGGAAAGATTGCTGGCTGGCGTGATCGTTTGTATCTTTCG
ATGGAACGTAATCAGGCATCTCGAACTGAGTCTTTCAAATTGCAACCAAGCAAAACCATC
ACGGTTGGAACAGAGCTGCACCTT
>RXN02348-downstream
TAATCAGGCAGTTGCTGGCCAAC
>RXN02354-upstream
GAATAAAGAAAAAGAAACTGGGCGGAACCAAGGATGAGAAACCCACCGCTAAGGATGGTG
TTGTAAAGGCCGATTCTGCTGTGAAGGAAGCCGCTAAGCC
>RXN02354
ATGACTAAACGAACAAAAGGACTCATCCTCAACTACGCCGGAGTGGTGTTGATCCTCTTC
TGGGGACTAGCTCCCTTCTACTGGATGGTTATCAGCGCACTGCGCGATTCCAAGCACACC
TTTGACAGCACGCCATGGCCAACGCACGTCACCTTGGATAACTTCCGGGACGCACTGGCC
ACCGACAAAGGCAACAACTTCCTCGCAGGCATTGGCAACTCACTGGTCATCAGCGTCACC
ACAAGAGCGATCGGTGTTCTCGTGGGAGTGTTCACCGCCTACGCTCTAGCCGGACTGGAA
TTCCCGGGCAAAGGCATTGTCACCGGCATGATCTTGGCAGCCTCCATGTTCCCCGGCATC
GCCCTGGTCACTCCGCTGTTCCAGCTCTTCGGTGACCTCAACTGGATCGGCACCTACCAA
GCGCTGATTATCGCGAACATTTCGTTCGGGCTACCTCTGACGATCTACACGCTCGTATGC
TTCTTCAGGCAAGTGCCCTGGGAACTCGAAGAATGAGCACGTGTCGACGGCGCGACACGT
GGCCAAGCCTTCCGCATGATCCTGCTTCCTCTAGCAGCGCCCGCACTATTTACCACCGCG
ATCCTCGGATTCATTGGAACGTGGAACGAATTCATGCTGGCCGGCCAACTATCCAACACC
TCCACAGAGCCAGTGACCGTTGCGATGGCAAGGTTCACCGGACCAAGCTCCTTCGAATAC
CCCTACGCCTCTGTCATGGCAGCGGGAGCTTTGGTGACCATCCCACTGATCATCATGGTT
CTCATCTTGCAACGCCGCATCGTCTCCGGACTCACCGCAGGTGGCGTGAAAGCC
>RXN02354-downstream
TAGACTAGATACTCATGAGTGCT
>RXN02356-upstream
TTGGGAGTAGCCATGCGTTCTGCTCCTGACCTTGAACAGCGGTCCCAATTTAGACCCGCT
AAACCCACAATGTGTACTGGTGCTGGTAATTTAGTAGAAC
>RXN02356
ATGGCAACGGTCACATTCGACAAGGTCACAATCCGGTACCCCGGCGCGGAGCGGGCAACA
GTTCATGAGCTTGATTTAGATATCGCTGATGGCGAGTTTTTGGTGCTCGTCGGCCCTTCG
GGTTGTGGTAAATCCACTACGCTGCGTGCTTTGGCGGGGCTTGAGGGCGTGGAGTCGGGT
GTGATCAAAATTGATGGCAAGGATGTCACTGGTCAGGAGCCGGCGGATCGCGATATCGCG
ATGGTGTTCCAGAATTATGCTCTGTACCCTCACATGACGGTGGCGAAGAATATGGGTTTT
GCGCTGAAGTTGGCTAAGCTGCCGCAGGCGCAGATCGATGCGAAGGTCAATGAGGCTGCG
GAAATTCTTGGGTTGACGGAGTTTTTGGATCGCAAGCCTAAGGATTTATCGGGTGGTCAG
CGTCAGCGTGTGGCGATGGGTCGCGCGTTGGTGCGTGATCCGAAGGTGTTCCTCATGGAT
GACCCGCTGTCCAACCTGGATGCGAAATTGCGCGTGCAAACCCGCGCGGAGGTCGCTGCT
TTGCAGCGTCGCCTGGGCACCACCACGGTGTATGTCACCCACGATCAGGTTGAGGCAATG
ACGATGGGCGATCGGGTTGCGGTGCTCAAGGACGGGTTGCTGCAGCAGGTCGCACCGCCC
AGGGAGCTTTACGACGCCCCGGTCAACGAATTCGTTGCGGGCTTCATCGGCTCGCCGTCC
ATGAACCTCTTCCCTGCCAACGGGCACAAGATGGGTGTGCGCCCGGAGAAGATGCTGGTC
AATGAGACCCCTGAGGGTTTCACAAGGATTGATGCTGTGGTGGATATCGTCGAGGAGCTT
GGCTCCGAATCGTATGTTTATGCCACTTGGGAGGGCCACCGCCTGGTGGCCCGTTGGGTG
GAAGGCCCCGTGCCAGCCCCTGGCACGCCTGTGACTTTTTCCTATGATGCGGCGCAGGCG
GATCATTTCGATCTGGAGTCGGGCGAGCGTATCGCT
>RXN02356-downstream
TAGTTTCGGACGTGGGGAGGCGT
>RXN02391-upstream
CAAAGTGGCGATCCTGAATTTGCCATCGAATCTGCCGTGAGAAGAGTTGCAGAGCTGGCG
AGGCGGTAACGCTGAACGGCGGCGGGTAAGATATTTGAGC
>RXN02391
ATGACACAATCAGATTTACCCGATGATGTTCAGGAATTGGTCACTAAGATCTTTGGACTG
GCACGTGATGGGGGAGCAGAATCCGCAGCAACCCTCGGTGCATATGTCGACAACGGCGTT
GACGTTAACCTGTCCAACCAAGATGGCAACACTTTGCTCATGCTCGCAGCATATGCAGGA
CATGCTGATGTCGTGCAGGCGTTGATTGAGCGTGGCGCCGATGTGGATCGCGTGAACAAC
CGCAATCAGACGCCGCTGGCGGGCGCGATCTTTAAGAAGGAAGAAGCCGTCATTGAGGCA
CTGCTTGCTGGTGGTGCTGACCCATACGCTGGAACTCCAACTGCTGTTGATACCGCCAAG
ATGTTTGGCCGCGAGGATCTCGTAGCTCGCTTCGAGTCA
>RXN02391-downstream
TAGGCCGGTGGAGTGGACCGCTT
>RXN02442-upstream
GCCGTGATGTTGTTGAGCGCGATGTGATTGCCGTATGTGCATGTGAGATTCCGGACGCTG
AGTTCTGCCATTCCTTAATGATAACGGTTATCATTTTCAA
>RXN02442
ATGAAGTTTTTTACTGACGCCCTCATAGTGCCTTTTGACGTTTCATTCATCTCCCGCGCC
CTGGTCGCCGGATGCCTGGCCGCAATTTTATGCTCACTCATTGGAACGTGGGTTATTTTG
GGCAGGCTAACCTTTTTCGGCGACGCTATGTCGCACGGCTTGGTCGCCGGAGTAGCCACG
GCATCACTATTGGGCGGAAATCTCATGTTGGGCGCAGGAATGAGCGCATTAATCATGTCA
GCCGGAGTGGTGTGGACCAGCAGAAAATCCAGCCTGTCCGAAGACGTCAGCATTGGGCTG
CAATTTATTACCATGCTTTCCCTCGGCGTGGTTATTGTGTCCCACTCCGATTCCCACGCC
GTAGACCTCACCAGTTTCCTTTTTGGAGACATTCTTGGCGTGCGACCCTCGGATATATTC
ATCATCGCCATTGCAACAGTGTTGGGTGGATTGAGTATTTTTCTCTTCCACCGACAGTTC
ACTGCACTCGCTTTCGACGAGCGTAAAGCTCACACCTTAGGACTCAATCCCCGCTTTGCA
CACCTACTCATGCTGGCACTGATCGCATTAGCTACGGTGGTGTCGTTTCAGGTGGTGGGA
ACGCTTTTAGTGTTTGGACTTCTGATTGGTCCGCCCGCCACGGCTGCACTTTTAGTGCAA
GACAAAGCAAGTATTTCACTGATCATGATGGTCGGGTCGCTTCTTGGATGCGCGGAAATT
TACCTCGGGCTTTTAATCAGCTGGCACGCAAGCACTGCCGCGGGAGCCACTATCACTTTG
TTAAGTGCTGCGATATTTTTTGCCACCTTATTGACAAAGAGTGCGATTAGTAGGTTAAAC
TTCACCGCG
>RXN02442-downstream
TGATACTGAAAGACATTTTCAAT
>RXN02447
ACAGTAGTTCCGGTGTACCTCGCTGAACTGGCACCACTAGAAATCCGCGGCTCCCTGACC
GGCCGAAACGAGCTTGCTATCGTCACCGGCCAGCTGCTTGCCTTCGTGATCAACGCGCTT
ATCGCCGTCACCCTACACGGAGTTATTGATGGAATCTGGCGCATCATGTTCGCCGTCTGT
GCCCTCCCTGCCGTCGCCCTCTTCCTCGGCATGCTGCGGATGCCGGAATCACCACGCTGG
CTGGTCAACCAGGGGCGTTACGACGACGCCCGCCGCGTCATGGAGACCGTCCGTACCCCT
GAGCGTGCGAAAGCCGAAATGGATGAAATCATCGCGGTGCACTCTGAAAACAATGCGGCA
CTTCCTGGTGTTAAGCAGTCTTCGGGCCAGGCTTCAGGCCAGGTTTCTAGCAAGCACACC
CACATGTCCATCGGCGAAGTCCTCAGCAACAAATGGCTGGTTCGTCTGCTCATCGCCGGC
ATCGGTGTTGCAGTTGCCCAGCAGCTCACCGGCATCAACGCCATCATGTACTACGGAACC
CGCGTCCTCGAGGAATCCGGCATGAGCGCAGAAATGGCTGTGGTTGCCAACATTGCTTTC
GGTGCCGTTGCCGTCATCGGTGGACTGATCGCACTGCGCAACATGGAGCGCCTGGATGGC
GGCACCACCTTCATCATCGGCCTGTCACTGACCACCACCTTCCACCTTTTGATCGCAGCT
GCCGGCACTCTCCTTCCAGAAGGTAACTCCATTCGACCATTCGCCATCATGATCCTTGTT
GTTGGGTTCGTGCTCTCCATGCAGACTTTCCTCAACGTTGCAGTGTGGGTGTGGCTGGCG
GAAATCTTCCGAGTCCGAATGAAGGGTATCGGCACCGGTATTTCGGTATTCTGCGGTTGG
GGCATCAATGGCGTCCTAGCGTTGTTCTTCCCAGCACTGGTCTCCGGCGTGGGTATCACC
TTCTCCTTCGTTATGTTCGCAGTGGTCGGAGTCATTGCCCTGGCGTTCGTCAGCAAGTTT
GTTCCTGAAACCCGTGGCCGCTCACTTGAAGAACTCGATCACGCAGCATTCAGCGGCCAG
ATCTTCAAGAAGGCT
>RXN02447-downstream
TAAACCCCCTCCGATCTCTTTGG
>RXN02455-upstream
AAGCCTTCGTTATGGGAGGTCTCCCAGACACAATCGAATACGGGCCGGATATCCATCTCG
GCTCATCACCCCGCTTTTTATCAAGAAAGATGAGGACCTC
>RXN02455
TTGAAGCGTCTTACTCGCATCGCATCCATCAGCATGGCCTCCATGCTCGCCGCCGCAAGT
CTCGTCGCGTGCTCCGGCTCCACCGACGAGGAAGGCGATGTTTACTTCCTGAACTTCAAG
CCTGAACAGGACGTGGCATACCAGGAAATCGCAAAGGCCTACACTGAAGAGACCGGCGTT
AAGGTCAAGGTCGTTACTGCCGCCTCCGGCTCCTATGAGCAGACCCTCAAGGCCGAGATT
GGCAAGGACGAAGCCCCGACTCTCTTCCAGGTCAATGGCCGAGCCGGCTTCATCACTTGG
GAGGACTACATGGCAGATATGTCGGACACCGAGGTAGCTAAGCAGCTGACCGACGACATT
CCGCCGGTGACCACCGAGGATGGCGAGGTACGTGGCGTTCCGTTCGCCGTCGAGGGCTTC
GGCATCATGTACAAGGACGAGATCTTCGACAAGTAGATCGCCACGTCCGGCGGAAAGATC
AAGTCCACGGATGAGATGACGAGCTACCAGAAGCTCAAGGAAGTCGCCGAGGATATGCAG
GCAAAGAAGGACGAGCTGGGTATCGAAGGCGCCTTCGCCTCCACCTCGCTGACATCGAGT
GAGGACTGGCGTTGGCAGACCCACCTGGCCAACGCTCCGATCTGGCAGGAGTACCAGGAC
AAGGGAGTCGAGGACACCAACGAGATCGAGTTCTCCTACAAGAAGGAGTACAAGAACCTT
TTCGATCTCTACCTTGAGAACTCCACCGTAGAAAAGTCTCTTGCGCCGTCTAAGACGGTG
TGTGATTCCATGGCTGAGTTCGCACAGGGCAAGGCCGCTATGGTTGAGAACGGTAACTGG
GGATGGTCCCAGATTTCCGAGACTTCTGGCAACGTGGTGAAGGAAGACAAGATCAAGTTC
CTGCCCATGTACATGGGTCTGCCAGATGAAGAAAAGCACGGCATCAACGTCGGTACCGAG
AACTATTTGGGCGTGAAGTCTGAGGCCTCCGAGGTCGACCAGCAGGGCACCAAGGACTTC
GTGGATTGGCTGTTTACCTCTGAAGCTGGCAAGGAGGACGTGGTGAAGGACCTTGGCTTC
ATCGCACCGTTCGAAAGCTACACCGCTGAGAACACCCCGAATGACGCCCTTTCTGAGCAA
GTCGCGGAAGCTATCGCTAACAAGGATCTGACCACCTACCGGTGGAACTTCCAGTACTTC
CCGTCCCAGCAGTTCAAGGATGACTTCGGCCAGGATCTGTCGCAGTACGCCTCCGGAAAG
CTGAAGTGG
>RXN02515-upstream
GTGGCTAAGCACAGTTACTTGGCCAAGCTGGGCGGCAGAAAAACCGGCCCAGCTAATACT
TCAGTTTAAAATTCGCTTCAACCCTGAAAGATTGTGACAG
>RXN02515
ATGAGCACTCTTGAAATCCGTAACCTGCACGCACAGGTCCTGCCGTCCGATGAGTCCGCT
GAGCCTAAGGAAATCCTCAAGGGCGTCAACCTCACCATGAACTCTGGTGAGATCCAGGCC
ATCATGGGCCCTAACGGTTCCGGCAAGTCCACTCTTGCTTACACCCTTGGTGGACAGCCA
CGCTACGAGGTAACCGCAGGCGAGGTCCTCCTCGACGGCGAGAACATCCTGGAGATGGAA
GTTGATGAGCGTGCACGCGCTGGTCTCTTCCTGGCCATGCAGTATGCAACTGAAATGCCT
GGCGTTTCCGTTGCTAACTTCCTGCGTTCCGCAGCGACCGCAATCCGCGGCGAGGCTCCT
AAGCTTCGCGAGTGGGTTAAGGAAGTGCGCACCGCTCAGGAAGCTCTGGCAATTGACCCT
GAGTTCTCCAACCGCTCAGTCAACGAAGGTTTCTCCGGTGGCGAGAAGAAGCGCCACGAG
GTTCTGCAGCTTGATCTGCTGAAGCCAAAGTTCGCGATCATGGATGAGACCGACTCCGGC
CTTGACGTGGATGCACTGCGCATTGTTTCCGAGGGCATCAACTCCTACAAGCAGGAGACC
GAAGGTGGCATCTTGATGATCACCCACTACAAGCGCATCCTCAACTACGTTAAGCCTGAC
TTCATTCACGTTTTCGCGAATGGCCAGATTGTGACCACCGGTGGCGCTGAGCTTGCTGAC
AAGCTCGAGGCTGACGGCTACGACCAGTTCATCAAG
>RXN02515-downstream
TAACATGTCCGATTTCCTCAATG
>RXN02549-upstream
GCAGTCGCAGTAGTTGGGGTTTCAATGATCTCAGGGCAGGACACTGTTCCCACTGGTAAC
GCCGTAACTGCAGACGATGCCCTGCTCGGTGGCCCTGAGT
>RXN02549
ATGGTTCACGCGAAGCAGACTAAGAAGCCACTTCCCCGTTTTCTTCACTCGGCGCATTTC
TATGTGTGGATTGTGCTGGGTTTTGTGGTGTTTGCGCAACCTTATGGTCAGGTTGCTGCC
GATACTAAACTAGATTTGCTGCTCAACCGCGGAGGATTTTTAACCGGTGCGCTTCATGCG
TGGACTGACACGTTCAGCTTGGGTCAGTTGGAAAACCAAGCTTATGGCTATCTGTTTCCC
GAAGGGTTTTTCTTCCTCATAACTGATTTCCTCCCTGACTGGATTGCGCAGCGACTGTGG
TGGTGGCTTGTTCTTGGCCTGGGATTTTCTGGATTCTACGCACTGGTAGCCCGGCTGGGG
ATTGGCAATCCTGCATTCAGGGTGATCGCCGCGCTGCTGTTTGCTCTGTCCCCGCGCACG
CTCACCACCCTCACTGCAATCTCCTCCGAAACTTGGCCTATCATGCTCGCGCCATGGGTA
TGTCTGCCTCTGCTTTCGCGAAATGTGGATGCACGGGCCATCGCGTTGTCCTTACTTCCC
GCGGCATGCATGGGTGCAGTTAATGCCACCGCCAGGATGGCAGCACTCATCCCGGCAGCG
CTGATCTTGCTGTATAGAGGGCTCTTCTTAAGGCTGCTTCTGTGGGGAATGGGCGTTCTC
GCTGTTAATTCATGGTGGATCGGACCTTTGTTGGTGCTTGGCAAATACGCCCCGCCCTTC
ACCGAATTCATCGAAAGTTCCTCCGTCACCACTTCCTGGCTCAACCCAGTAGAAATACTC
CGCGGAACCACCAGTTGGACACGCTTCGTAGACACTGAACGACAAGCCGGATATCTCCTG
GTCAACGATGCTCTCTTTGTCACCCTCAGCGTTCTCGTCGCAGCCCTCGGCTTGATCGGC
CTCACCTTGATGAAACACCGTGGACTGTGGGCATTCATGCTGGCCATCGGACTCCTCATC
CTCGGCAGCGCCCACCTAACGGCTGTTCAAGAATTCCTCGACGGCCCAGGCGCAGCACTT
CGAAACATGCACAAATTTGATCTATTAGTCCGCATGCGGTTGATGGTGGGCGTTGCCGCA
TTGGGGTGGCATATCAGTCTGCCCTTGCTTGGGACGACTGCATTGACCAGCGGACAAGGC
AAACACCACACCATCCCGCTGCCTCTCCAAAAAGGCCAAGCCGCAGGACTCCTCGTGGTG
ATCATCGCTGTCGGTGCTCTTGCTCCGGCATGGTCGGCACGGCTGCTACCTCAGGGAACG
TGGGATGAAGTGCCTGACTACTGGTACGAAGGCACAGAATTCGTCAACCAAAACGCGACA
GGCACCCGGACGTTGATTTGGCCTAGGTGGGCGTTTGCCCGCCAGGACTGGGGATGGAGT
CGGGATGAAGCAGCTCAACCACTTCTTGATGTTCCGTGGGCTGTGCGCGATGCCATTCCT
TTGGTTCGGCCGGAGGCGATTCGCGGATTAGATGGTCTCGACGACCTAGGCACTGTAGGC
AGCGGTCTAAACGACGAGGCTTTAAAACGTCTAGGCATCGGCGCAGTACTGGTGAGGCAT
GATCTGGAAGCCGAGCCAGATATTGAGGTGGATCTGCCTGGGGAAAAGCACACTTTTGGC
TGCCAAGGCCAAGTAGACGTCTACCTCACCGACCCCGACCGCAATATGTGGATCACTTGC
GGCAGATCCAAGCAGCTGCCCACCGTCGCTGGCGGCGGCGAAATGCTCTCGCTGCTAGAC
ACCATCAACGGCTATTCCCCGAGGACTTTGGTGAGTGAGAATGCGCAGATCGTCACCGAT
ACCCCTCAGCTAGTCGGCACAAATTAGGGCGATGGCACCAGTTCCGCAGCATTGGCCAGC
CTTGATGAGACTGAGGTGAAAAACCGGATCGTGGATTATCCTTCCGCGGGGCCAATGACG
CAGGTGGTGCAGGAAGGTTCCATCACGGCGTCTTGGTCTGGTTCCGATGCGACTTCTTTC
GGGGGCGCGGATCCTGATCGTTCCCTTAATTCACTTCTTGATCATCGTTACAACACGGCC
TGGTACCCGACACGTGGCGATACGTCTCCGTGGCTCGAAGTCTCCGGTACCGGCACCACA
TTATGGATCTCCCCCCGCAGCACCGTCACCGCCACCATCACCTCCGGCGATTCCGTGATG
GTCCGCGAGTTGGAAAAAGGCCGCACCAGCACAGTTACGTTGGCGGAGCCTGAAGCTCGC
ATTGAATTCGATGGTTTCGTAGGAATTTCCGAGCTGTCCCTAGAGGGTCTCAGCCGCACC
ATCACTGTGCCGGAGACCTCTCCTGAGGTGCAGCAATTCGTTTTCCAACGCCTCACAGTG
CCCACCTCGTTCCTCGACCGCACTTTCACAGTCCCCCGCCACATGTCCGTCACCGTGGAG
GCCCAATCCTGCGTCACATTGGAACTCGACGGCGATCGCATCGACTGTGGCCCCTCGAAC
TCACCCCCGGAACCCACACCCTGCGCACCCAATCGGAATGGGTCACCCTCACCGAATCCG
CTCCGCTCGCCGCTGTTCAGCCAGCAACAAACATCGAGGCAGCACCCACCGACCGCGTGC
TCGTCACCACGCGCGCTTTCAATTCAGGTACCAGCGCGCTTATCGACGCCACCCCCCTTT
CGCCAATCCAACTCGACGCCTCCTCCCAAGGTTTCATCATCCCCGCGAACGCCTCCGGCG
AGT
>RXN02549-downstream
TGAGCTTCGCTTTCGACGGCGAA
>RXN02570-upstream
CCATTGTTATGCTCATTGTGTTTGTGGTGGTCAAGTCGCTACCCAAGCGCACCACTAGGG
CATTGGTTCCGCAGCGGGTTCCGGAGGACGTCGCTTAAAC
>RXN02570
ATGAATCCTTTGACATGGATCATTGGCGCATTCAGCATGTGGATCGTGGTGCTGGGCGTT
AATAAGCTTGGTTTAAGCATCGCAGTGATCATCATCGCGCAGGTCGTGGCCATGATTCGG
GTGCGCAATGTATCTGTGTTGGCTTCAACAGCATTGTTATCGGTTCCTGCATTGGCCTCG
ATGGCGCTGATTCACATGCCGTATTCTTCCGACGGCTGGTTGATTGCTCTTACCTTGACG
GCTCGTTTTAGTGCGTTGATGTCTATTTTCCTCCTTGCAGCAACAGCGATTACTATTCCT
GAGCTGGTGAAATCCCTATATCGTTGGCCCAAGCTGGCGTATATCGTGGGTTCTGCATTG
CAGATGATTCCGCAGGGTAAACAGACCTTGGCGTTGGTTCGTGATGCCAATGCTTTGCGC
GGGCGCAGCGTTAAACGTCCCGTGCGCGCGGTGAAATATGTGGGTTTGCCCCTGATTACA
CATTTACTTAGTGCAGGTGCCGCGCGAGCGATTCCCTTGGAGGTCGCAGGCCTGGACAGG
CCGGGGCCGCGTACGGTGTTGGTTGAGGTGGTGGAGGGGCGCGTCGAAAAGCATTGTCGC
TGGTTGTTGCCGCTTTTGGCAGTCGGGATGGCGTGGTGGCTC
>RXN02570-downstream
TAACTCAAATCGTCGGACCGTCC
>RXN02595-upstream
GTGGGTAAAGGGGACTCCGAGGAAGTCCACGTCGTCTTCTTTCGCGGCGCTGAGGATGGT
TTCGCGGATTTGTGCGGGGGAGTGGGTGGCAGAGAAAACG
>RXN02595
GTGATCGTTGTGGCCATGGCTTCCATTATGGCTTGTTTAAAAGCAGCTAGACTGAATAAC
CCTATGAAGATCCTTTTGTTGTGCTCGCGTGATACCACTCATCCTCAAGGTGGCGGAAGT
GAACGCTATCTCGAGCGGGTGGGTGAGTTTTTGGCGGATCAGGGCCATGAGGTGGTGTTT
CGTACTGCTGGGCACACGGATGCGCCACGGCGTTCTTTCCGCGATGGTGTGAGGTATTCC
AGGAGCGGTGGGAAGTTTAGTGTGTATCCCAAGGCGTGGGTGGCCATGATGTTGGGTCGT
GTGGGGATTGGCACGTTTTCCAAGGTTGATGTGGTGGTGGATACGCAGAATGGCATTCCG
TTTTTTGGAAAGTTTTTCTCCGGTAAGCCGACTGTGTTGCTCACGCATCATTGCCATAAG
GAGCAGTGGCCGGTGGTGGGTCGGGTGCTGGCGAAGGTTGGTTGGCTGATTGAGAGCCAG
ATCGCGCCGCGCGCTTACAAAACTGCGCCGTATGTGACTGTTTCAGAGCCGAGCGCTGAG
GAGCTCATTGCGTTGGGTGTGGATCAGCAGCGGATTCATATCGTGCGCAATGGCGTGGAT
CCCGTGCCGCTGCACACGCCGAAGCTGGATCGCGATGGCCAGCATGCGGTGACGTTGTCG
CGCCTGGTTCCGCACAAGCAGATTGAGCATGCGATGGATGTCGTCGCGGCGCTCGACGGC
GTGGTGCTGGATGTAGTCGAAAGCGGTTGGTGGCAGAAGGAACTGGTCGATTATGCCCGC
ACGCTGGGTGTGAGCGATCGCGTGGTTTTCCACCGCCAGGTCGCCGAGGATCACAAGCAC
GCCCTGTTGGAGCGCGCCACGATTCATCTCATGCCTTCGCGCAAGGAAGGCTGGGGCCTG
GCGGTCACGGAGGCGGCGCAGCACGGCGTTCCGACGATCGGTTACCGAAGCTCAGGCGGC
CTGCGCCATTCCGTCGTCGACGGCGAAACCGGCCTGCTTGTCGACTCCAAGGCCGAGCTT
ATTTCAGCCACCAAAACCCTGCTTATCGACGCCTCCCTCCGCTCCAAGCTCGGCGCCAGC
GCGAAGCAGCGCGCCGAAAACTACAAGTGGGACACCGCGGGAGCGCAGTTCGAGGAACTA
CTTCTTGGTCTTGCGTCGAAAAAG
>RXN02595-downstream
TAGTCCCAGCGGCAACGCCATCC
>RXN02614-upstream
TCATTGTATACGCCACCCTCGGTCTGCTGTCTGAAGCGCTGATCAGAGCTTGGGAACGTC
ACACCTTCCGCTACCGAAACGCATAAGAAAGTTGCTCGCC
>RXN02614
ATGACTGCCACATTGTCACTCAAACCCGCAGCCACTGTCCGTGGATTGCGCAAATCATAC
GGAACTAAAGAAGTCCTCCAAGGAATCGACCTCACCATCAACTGCGGCGAAGTAACCGCG
CTGATCGGACGGTCAGGTTCAGGAAAATCCACCATCCTGCGCGTGTTGGCGGGCCTATCT
AAAGAGCATTCCGGCTCTGTAGAAATTTCCGGAAACCCGGCCGTTGCCTTCCAAGAGCCT
CGCCTGTTGCCGTGGAAAACGGTGCTCGATAATGTGACCTTTGGCCTCAACCGCACTGAT
ATTTCGTGGTCAGAAGCACAAGAACGCGCCTCGGCACTGCTTGCAGAAGTCAAACTTCCC
GACTCCGACGCCGCCTGGCCCCTCACGCTCTCCGGCGGCCAAGCCCAGCGCGTCTCCCTT
GCGCGAGGGCTCATCTCCGAGCCAGAGCTTTTGCTTCTCGACGAACCCTTCGGCGCCCTC
GATGCTCTGACAAGACTGACAGCCCAAGACCTGCTGCTCAAAACCGTGAACACCCGAAAC
TTGGGAGTTCTGCTGGTCACCCATGATGTTTCCGAGGCCATCGCCCTGGCCGACCACGTC
CTTCTTCTTGACGACGGCGCCATCACACACAGTTTGACTGTAGATATCCCCGGCGATCGC
CGCACCCACCCCTCCTTTGCCTCCTACACCGCTCAACTCCTTGAGTGGCTCGAAATCACC
ACACCTGCG
>RXN02614-downstream
TAGAAAGAAATCATGAAATTTAA
>RXN02795-upstream
GCGGTGTGGCCCGGTGCTGCGATCGCTTTGACGGTCCTTGGTTTTAATCTTTTCGGTGAT
GGTTTACGCGATGCCATCGATCCAAAGCGGGAGGTCGGCC
>RXN02795
GTGCTTAAAGTTTCTGATTTAACGGTTGGCAACAATTTTGTCCACAACGTCTCCTTGGAG
GTCAACCCGGGCGAACGCGTCGGCATCATCGGCGAGTCCGGCTCAGGCAAATCACTCACC
GCGCTATCCATCATGGGTTTAACTGACCTGCCGACCACCGGCCAGATCACCTTCAACGGC
AAACCCTCCGCTACATTCCGTGGCACCCGCATCGCCATGGTTTTCCAAGAACCAATGAGC
GCACTCAACCCGCTCATGCGCATCGGCCGCCAAATCGAAGAAATGATGACCCTGCACGGG
GCAAGCAAAAAAGACGCGCGGGCGCGCTTAAAAAGCTTGCTTATCGACGTCTCCCTCCCC
GAACGCACCGCTTCGGCCTACCCACACGAACTTTCAGGCGGGCAACGCCAACGCGCACTA
ATCGCAATGGCGCTGGCCAATGATCCTGACCTGTTGATCTGCGATGAACCCACCACGGCT
TTGGATGTGGTTGTGCAAAAACAAATCGTCGATCTGCTGCTGCGTCTCACCAAAGAACGT
GGCACCGCTTTATTGTTCATCACCCACGATCTTGGACTCATCGCGCGCACCTGCGAACGC
TTATTGGTGATGAAATCCGGCGAAACCGTAGAACGCGGCGACACCGAGGCAATTCTTCGC
TCCCCCGCCCATTCGTATACCCAACAGCTCCTTGATGCTTCAATCCTTGACCAGCCAGAA
ATCGCCTCAGATTCTGGCGCGCCGGTAGTGATTGATGTGGAGGAGGCGTCGAAAAGCTTT
AAAGAAACCACCGCCCTCCACAAGGTTTCATTGGCGGTGCGCAAAGGTGACCTGCTTGGA
ATAGTCGGCGGATCAGGTTCCGGCAAAACGACTCTGCTGAAGCTGATCGCCGGTTTGGAT
AAGCCCACAAGCGGTACCGTTGCGGTAACCGGTGGTGTGCAGATGGTGTTTCAGGATCCC
CAATCAAGCCTCAACCCACGGATGAAAATCAAAGACATTGTCGCCGAACCACTGCTTGGT
TGGAAGGCGGCGGAGAAAACCACACGGGTTGCGGAAGTCATCACCCAAGTGGGACTGAGC
CCCGATGTCTTAGATCGCTACCCCCACGAATTCTCCGGAGGACAGCGCCAACGAATCTCC
ATCGCCAGAGCCCTCGCCATCAAACCAGCGATCCTGCTTGCCGACGAACCTGTCTCCGCC
CTCGATGTGTCCGTACGTAAACAAGTACTGGATGTTCTCCAACAACTCGTCGAAGAATAC
GGCATCACCTTGGTGTTCGTCTCCCACGATCTGGCAGTGGTGAGACACCTGTGCACAACC
GTGTGGGTGATGGAACAGGGACGAGTCCTTGAGCAAGGGCCCATCGATTCGGTTTATGAT
CACCCACAGACCGAATACACCAAGGAGGTGGTTGATGCCGTTCCGCGGTTGAGGCTT
>RXN02795-downstream
TAAACCAGCGCAGATGACAACGC
>RXN02925-upstream
AAACCGTCCACCGGGCAATTGAGGAGACCGGCTACACCGTCTTGTCCTGATCGATTCACC
CATCATCTCGACCCCGACCGGGTTGAGCGGAAGGAACCTC
>RXN02925
ATGAGCACTCCCCAGCACCACGGTGATCACCCCGCTCCGGAAACAGACCACACCCACCAC
CCGAATCATGCCGGTCAGGAGCACCATGCGGATGCCGCCACCCACGGCCAGGCCATGCCG
CACGATCATCCGCATTCCACTGTCGATGAAGAACATCAGGTCCACAGTCACGGTGAACAC
GCCGGCCACAGCGCCGCGATGTTCCGGGACCGCTTCTGGTGGTCGCTGATCCTGTCGGTT
CCGGTGGTGTTCTTCAGCCCGATGTTCGCCGACCTGCTGGGATATAATATTCCGGAGATT
CCGGGAGCCTACTGGATTCCTCCGGTCCTGGGCACGATCATCTTCCTCTACGGCGGCACC
CCCTTCGTCAAGGGCGCAATGACCGAGCTGAAATCCCGCCAACCGGGCATGATGCTGCTG
ATCGCCATGGCGATCACCGTGGCGTTTATCGCCTCCTGGGTCACCACCCTGGGGCTGGGC
GGGTTCCACCTAGATTTCTGGTGGGAACTGGCCCTGCTGGTGACCATCATGCTGTTGGGC
CACTGGCTGGAGATGCGCGCTCTTGGTGCAGCCTCCTCCGCGCTTGACGCGCTGGCAGCG
CTCCTGCCCGATGAGGCCGAGAAGGTCGTCGACGGGACCACCCGCACCGTAGCGATCTCA
GAGCTGGCCGTCGACGATGTCGTGCTGGTCCGAGCAGGTGCCCGGGTCCCGGCCGACGGG
ACCATCATCGACGGAGCGGCCGAATTCGATGAGGCCATGATCACCGGCGAATCCCGACCC
GTCTACCGGGATACCGGTGAGACCGTGGTGGCCGGCACCGTGGCCACCGACAACACCGTC
CGTATCCGGGTGGAGGCCACCGGTGGGGACACCGCCCTGGCAGGCATCCAGCGCATGGTC
GCCGACGCCCAGGCCTCCTCCTCCCGGGCCCAGGCCCTGGCCGATCGAGCCGCAGCCTTA
CTGTTCTGGTTCGCCCTGATCACGGCCCTGATCACCGCCGTGGTCTGGACCATCATCGGC
AGCCCCGACGATGCCGTGGTCCGCGCGGTGACCGTGCTGATCATGGCCTGCCCGCACGCC
CTGGGCCTGGCCATCCCGCTGGTCATCGCGATCTCCTCCGAGCGCGCGGCGAAATCCGGG
GTGCTCATCAAGGACCGCATGGCACTCGAGCACATGCGCACCATGGACGTCGTCTTGTTG
GATAAGACCGGCACCCTGACCGAAGGCGCACACGCCGTCACCGGCGTGGCTCCGGCCACG
GGTATGGCCGAGGGTGAGCTGCTGGCCCTGGCCGCCGCCGCTGAGGCCGATAGTGAGCAC
CCCGTGGGCCGCGCGATCGTGACTGCCGCGGCCGCACACCCGGAGGCCTCGCAGCGTCAG
CTGCGCGCAACCGGTTTCACCGCCGCGTCCGGCCGCGGGATGCGGGCCACCGTCGAGGGT
GCCGAAATCCTCGTGGGCGGGCCGAACATGCTACGCGAGTTCAATCTGACCACCCCGGGT
GAGCTCGCCGACATCACCGGTTCCTGGGCACAGCGAGGTGCCGGAGTGCTACATGTCGTC
CGCGACGGTGAGATCATGGGTGCGGTGGCAGTGGAGGACAAAATCCGCGGCGAATCCCGC
GCGGCGGTACGGGCCCTGCAGGCCCGCGGGGTGAAGGTGGCGATGATCACCGGTGACGCC
ACCCAGGTCGCCCAGGCAGTGGGCAAGGATCTGGGGATCGATGAGGTCTTCGCGGAGGTT
CTGCCGCAGGACAAGGACACCAAGGTCACCCAGCTGCAGGAGCGCGGTCTGAGCGTGGCC
ATGGTCGGCGACGGTGTCAATGACGCCCCGGCCCTGGCCCGGGCCGAGGTCGGTATTGCG
ATTGGCGCGGGTACAGATGTGGCGATGGAGTCCGCCGGGGTGGTCCTGGCCAGTGATGAT
CCCCGGGCCGTGCTGTCGATGATCGAGCTCTCCCATGCCAGCTACCGCAAGATGGTCCAG
AACCTGGTCTGGGCGACCGGGTACAACATCGTGGCCGTTCCGCTGGCCGCCGGTGTGCTC
GCCCCTATCGGTGTGCTGCTTCCCCCGGCGGCGGCCGCCATCTTGATGTCCCTGTCCACG
ATCATCGTCGCCCTCAACGCCCAGCTGCTACGCCGGATCGACCTGGACCCGGCTCACCTA
GCTCCGACCGACGGGAAGGAGGAGAAGGCTGCTGTGAGCTCTGCAGCCCCCGTCCGC
>RXN02925-downstream
TGACTTTCAATGCTTCATGGACT
>RXN02933-upstream
TGATCTGCTGTATGAGGTGGTTGATGCAAGAGTCGGTGCTGTTGGGGTTGCTAGCACTAA
GGTTCCAGGGAGCGTGGCTTAAGTGACAACGATCAAAAAC
>RXN02933
ATGCCCCTTTCAGGGAAAATCGGCGGCTTCATCGTTGCCGTTGTATTTGTTCTTGCTGCG
GTGTCTTTCATTTGGACTCCGTTTGATCCAGTTCAAGCTTTCCCACAGGAGCGCCTTGAG
GGAAGTTCTTTGAGGCACCTGTTGGGAACGGATGGTTATGGTCGCGATGTTTTATCCCAG
ATCATGGTTGGTTCCCGCGTCACGTTGTTGGTGGGCATCATTGCGGTGGCGATGGCAGCA
TTAATCGGCACGCCACTGGGTATTGCTGCGGGAATGCGCCGTGGCATGGTGGAAACCTTT
GTCATGCGTGGTGGCGATTTAATGTTGGCGTTCCCAGCACTGTTGTTGGCGATTATTTCC
GGCGCCGTTTTCGGGGCCTCCAGGTGGTCCGCGATGGTCGCGATCGGCATCGCAGGCATC
CCTAGTTTTGCGCGCGTGGCTCGTGCAGGCACATTGCAGGTGACCAGTCAGGATTTCATC
GCAGCTGCTCGGCTATGAAAAGTAAGTTCGGCCCGGATCGCGCTTCGCCATATTTTGCCC
AACATCACCAGCATGTTGATCGTTCAGGCATCAGTAGCTTTTGCCCTGGCGATCCTGGCG
GAAGCCGCATTGAGTTTCCTGGGTTTGGGCACCACTCCCCCGGATCCCAGCTGGGGTCGC
ATGTTGCAAACCGCTCAAGCATCCATCGGCGTCACCCCCATGTTGGCGGTGTGGCCCGGT
GCTGCGATCGCTTTGACGGTCCTTGGTTTTAATCTTTTCGGTGATGGTTTACGCGATGCC
ATCGATCCAAAGCGGGAGGTCGGCCGTGCT
>RXN02933-downstream
TAAAGTTTCTGATTTAACGGTTG
>RXN02945-upstream
TTCCGGTGCGATCCTTGCCGGCCTGCTCAGCTGGTACCTGGTCCGCGCGTTGGCGAGGAC
TGGTGGACTTGATCGTTTCGCCGCTGGCGGCGAGGTATAA
>RXN02945
ATGACCACCGCACTTGGAACGCGCGTTGTTGCGCGCAACTTTGGCTACCGCCATGCTTCC
CGGGAAAACCCCGCGCTCAAAGACATCAACTTCGAGATCGCACCTGGTGAACGCATCCTG
CTCACCGGCGCTTCCGGCGCCGGAAAATCCACGCTACTCGCCGCGCTCGCTGGCGTTTTA
GGCGCTTCTGATGAGGGCGTTTCTACGGGCGAATTGCTTGTCGACGCCCCCTCCATCGGT
TTGCTTCTCCAAGATCCACATTCCCAAGTCATCGCCTCCCGCATCGGCGATGATGTGGCG
TTTGGCTGCGAAAACCTCCAAATTCCGCGCGAGGAAATCTGGCCACGGGTGGAACGAGCA
CTTGAATTGGTGGGCTTGCATCTACCACTGAGCCACCCCACGAAATATCTTTCCGGTGGC
CAAAAACAACGCCTCGCTCTTGCCGGTGTGATCGCCATGGGTGCTCGTCTGATTCTGCTT
GATGAACCCACCGCAAACCTTGATCCTCAAGGCCAAAAAAATGTGGTCGCAGCACTGGAT
CGCGTTGTTCAGGAAACTGGAGCAACACTCATCGTGGTGGAACACCGCCATGAGCTGTGG
GTCAACATCATTGACCGGATCATCAGTATTACTGACGGCGAAGATGTCCAACCTGCAGAG
TTGATCAAGGTGGGCCAGTTGCCTGGGGCGCAGCCGTCGACAAGCAAACCGATCTTGTGG
GCGAATGATTTGCTGTGCACCTGGGGCGGCCTGCGTAGTTTTGAGGTGCCGGAAGGCGCC
TCGACGGTGATCACCGGGCCGAATGGCGCTGGAAAATCCACACTTGCGCTGACCATGGGT
GGATTGCTTCCGCGAAAAGTGGGCAGCTGGAACTCTCTGACACGGTGCGCGGCGGCCTTA
ACACGCCCCCGCACAAGTGGCGTTCAGCTGATC
>RXN02945-downstream
TAGCTGCACGTATTGGCACTGTC
>RXN02975-upstream
TCGTCGGTGCAGTCCTCGGATTGCTTAAGTTGTAGGTGGCTGGGGGCGTCGAAAAGCAGC
TTTATTGACCTGGCAACTTCAATTGATAGACTGTTAGGTT
>RXN02975
GTGATTGTCACCAATGATTTAGAGGTGCGCGTTGGCGCACGTACCCTTCTCGATGCCCCA
GGTCAGCTCCTTCGGGTGCAGCCAGGCGACCGTATTGGTCTGGTTGGTAGAAATGGTGCG
GGCAAAACCACCACCATGCGAATCCTCTCGGGCGAAACCAAGCCCTACGGAGCATCCGTA
ACCACATCTCGTGAAATCGGTTACCTGCCCCAGGACTCCCGCGAAGGCAACATCGAACAA
ACCGCCCGC
>RXN02994
ATCAAGATGACGGGAGTGCAAAAATACTTCGGCGACTTTCATGCCCTTACGGATATTGAT
CTTGAAATTCCCAGAGGACAAGTTGTCGTCGTACTTGGACCATCCGGATCCGGCAAGTCA
ACCCTTTGCCGCACGATCAACCGTCTCGAAACCATCGAGGAAGGCACCATCGAAATCGAT
GGAAAGGTTCTCCCAGAAGAAGGTAAAGGCTTAGCCAATCTCCGCGCCGATGTCGGAATG
GTATTCCAGTCCTTCAACCTCTTCCCCCACCTCACCATCAAAGACAACGTCACTCTTGCA
CCCATCAAAGTGCGAAAGATGAAAAAGTCTGAAGCCGAAAAGCTTGCGATGAGCCTGTTG
GAACGCGTCGGCATCGCAAACCAAGCTGATAAATATCCGGCGCAACTGTCCGGCGGTCAG
CAACAGCGTGTGGCCATCGCGCGCGCACTTGCGATGAACCCAAAGATCATGCTTTTCGAC
GAGCCCACCTCCGCCCTTGACCCTGAAATGGTCAACGAAGTGTTGGACGTCATGGCAAGC
CTTGCCAAGGAAGGCATGACGATGGTGTGTGTTACCCACGAGATGGGATTCGCACGCAAA
GCAGCCGATCGTGTGTTGTTCATGGCGGATGGGCTCATTGTGGAAGATACGGAACCAGAT
TCCTTCTTCACCAACCCTAAGTCTGATCGTGCAAAAGACTTCCTCGGCAAGATCCTTGCC
GAG
>RXN02994-downstream
TAGTTTTTGGCTGCGCCTCTATC
>RXN03020-upstream
CGCCGCAGCAGGACTCATCGGTGCCGCCATTTCACTCGGCCCCATCCTTCGCGTCGAACC
ACGCTCCGCACTCATGAACGCATAAGAAAAGGAACCTCAC
>RXN03020
ATGACTCTCCACGTTTCAAATCTCAATCTGACCGTCGCCGACGGATCCACCTCACGCACC
GTGCTCAACAACATACACTTTTGGATGTCCAACCAGGCGAAGTCGTCGGTATCACCGGCC
CATCCGGGTCCGGAAAATCCACCCTACTCGCCGTCCTCGGCTGCCTCCAAAGCGCCCGAT
TCCGGCACCGCGACGCTCGGCGACATCGACCTGGTCAACCCGCAAAACCGAGCTGCTTTA
CGACGCAACCACCTAGGAATTGTCTTCCAACAGCCAAACCTGCTCCCCTCGTTGACTGTC
CTCGACGAAGTGCTGCTCATTGCCCGGCTCGGCAGGATCCTCCCGGCCAGCCGCAGCGCA
CGCACCCAACACAAAGACAAAGCCCTTTCACTTCTGAACTCCATCGGACTCGGCGACTTA
GCAAAACGCAAGGTCAGCGAACTATCCGGTGGACAACAAGCCCGCGTTAACTTGGCCCGC
GCGCTGATGAACTCCCCCAAGCTCCTGCTTGTCGATGAACCCACCGCCGCCCTCGATCAA
CATTCCGCGAGCGAAGTCACCGAACTAATCGTCTCGATGGCCCAGCAATACAACGCCCCC
ACA
>RXN03080-upstream
CTTGCAAACAGGCGTGGTGGTGGCGTTCATTGGCTCACCAATTTTCCTTTATTTACTGCT
CAGCATGCGCAAGCGACGCGGATTGGGGCTGTAAAAACTC
>RXN03080
ATGCCTCAATTAGTTGAAATTCGTGATCTCAACGTTGAATTCCCCTCTCGCCATGCAGTG
AAAAACGTGTCTTTTTCTGCACCTGCTGGAAAAGTCACCGCACTGATTGGCCCAAATGGT
GCTGGTAAAAGTACTGCCCTTTCGGCGATTGCAGGATTGGTTGAATCCACCGGCGAGGTA
ATGGTTGGTGGGAGTGGGGTTGCGTCGAAAAGCGCTAAAGCCCGAGGCCGCCTGCTGTCA
CTCGTGCCGCAAAACACCGAGTTGCGCATTGGTTTTAGTGGACGCGACGTTGTCGCGATG
GGCCGCTACCCGCATCGTGGCCGCTTCGGCGTGGAGACCGACGCAGATCGACGCGGCACC
GATGACGCCCTGCGCGCCATCAACGCGCTCGACATCGCCGAGCAGCCCGTCAACGAATTA
TCGGGCGGCCAGCAGCAGCTCATCCACATCGGCCGAGCGCTCGCCCAAGACACCGCGGTC
GTGCTTCTCGACGAGCCCGTCTCCGCGCTTGATCTACGGCACCAAGTTGAAGTCCTTCAA
CTCCTGCGCGCCCGAGCTAATTCGGGCACCACCGTGATCGTCGTCCTTCACGATCTCAAC
CACGTTGCCCGTTGGTGCGACCATGCAGTGTTGATGGCCGACGGCGAAGTTGTCTCCCAA
GGTGACATCCGCGAGGTGCTCGAACCTGCCACACTGTCCACCGTGTAGGGACTGCCCATT
GCGGTGCGCGATGATCCCGAAACCAGCTCACTTCGCGTGATCCCGCATCCAAATCGCTTT
>RXN03080-downstream
TGATTGAAACTTTGACTTAAAAA
>RXN03081-upstream
ACGGACTGCCCATTGCGGTGCGCGATCATCCCGAAACCAGCTCACTTCGCGTGATCCCGC
ATCCAAATCCCTTTTGATTGAAAGTTTGACTTAAAAACCC
>RXN03081
ATGAAAAAATCACTCATCGCCATTGTTGGCAGTGCGCTCGTGTTAAGCGGCTGGACCTCT
GATTCTTCTGACTCTTCCGGCACTTCCGGAACTGTGGAAACCACTTCGATTACAACCAGC
GTTGCCGCAGCTGACGGCGCATTCCCACGCACCGTCACACTCGACGATTCCTCCATCACC
TTAGAATCCAAACCAGAGCGCATCGCCGTACTCACCCCAGAGGCAGCATCCTTGGTTCTC
CCCATCACAGGCGCCGACGGCGTCGTGATGACCGCCGAAATGGACACCGCTGACGAAGAA
ACCGCAGCTCTGGCCTCCCAAGTGGAATACCAAGTCAAAAACGGTGGCAGGCTCGACGCC
GAACAAGTTGTCGCCGGCGACCCAGATTTGGTGATCGTCAGTGCGCGTTTCGATACCGAA
CAAGGCACCATCGACATTTTGGAAGGCCTCAACGTCCCG
>RXN03081-downstream
TAGTTAACTTCGATTCAGACGCT
>RXN03108-upstream
CAACCAGCCTGCCACGTTGCTGGATGGACCGTCGTAATCATCGGCATCACCGGCTTAATC
CTTGACCACCTCATCGGTGAGTTGCAGAAAGTAGTTCGCT
>RXN03108
ATGACTAAACCAAACGCTTCCGTCGAGCTGAATACGATCACCAAGTCCTACGGCTCCACC
ACTATCATTGGCGATACGAGCATCACCATCAACGACGGTGAATTCGTCTCCCTCCTCGAC
CCTTCCGGCTGCGGAAAATCAACAATTCTCAAAATGATCGCCGGACTGGCCTCCCCATCC
ACCGGCACAGTCAGCGCAGGCAACGAAGAAATTAAAGGACCAGGACCTGACCGAGGCATG
GTTTTCCAAGACCACGCCCTCCTGCCC
>RXN03108-downstream
TGATTGACCGCACGCGGCAACAT
>RXN03116-upstream
AGGGGAATGGCTTAATATCGTAGGTCCCAACGGCTGCGGAAAGAGTACGTTGCTGCACGC
TTTTGCTCAGGTACTGTCACTGGAATCGGGAAGGCTGAAA
>RXN03116
ATGGGGGAGGGGGACGTCGAAAAGGATTTTGCTTTTGGTCTTAAAGCTGCGAAGCAGCGT
CGCTTTTTCGCGCGTACCGTGGCGCTCATGCCACAGAATCCTACTATTCCTGCAGGTCTG
AGCGTTTTTGATTATGTGCTGCTGGGGCGTCATCCGCACAGTTACGCGCCGGGGCGTGCT
GATGATGAGATCGTGAAGCGGTGCCTCGCTGATCTGAAATTGGAGCATTTCAGCGACCGC
GGCTTAGACGAATTGTCCGGCGGCGAGCGTCAACGCGTCAGCCTTGCCCGCGCGCTCGCC
CAAGAACCGCGCATCGTGCTTCTCGACGAGCCGACCTCCGCGCTTGACATCGGCCATGCG
CAGGAAACGCTTGAGCTTATCGACGCCATCCGGCACCGACTCGGCCTCACCGTGATCGCG
GCGATGCATGACCTCACCCTGACTGCGCAATACGGCGATCGGGTGCTCATGATGAATGGT
GGCCGCAAAGTTTTCGAGGGCACTGCAGCCGAAGTGCTCACCGCGGAGCGGATTTCGGAG
ATTTATGATGCCACTGTGATTGTTGAGGTTATTGATGGGCGTCCCGTGGTGATTCCGCAA
CGGTCGCAC
>RXN03116-downstream
TGACCTGTTGTGGCAGACGAGAC
>RXN03129-upstream
GCTGAGGTTGAGACCAAGCTGAACACCATCTACACCCGCGACATCGAACCACTTATTTAA
TCCGAGCACTTCAGCTACACCTATTTAAGGAGGCTGTGAC
>RXN03129
ATGGCGTCAATCGTCTTTGAAAACGTCACACGCAAATACTCTCCGGGCGCACGCCCGGCC
GTCGACAAGCTTAATTTGGAAATCGCCGACGGCGAGTTCCTAGTTCTCGTTGGACCCTCA
GGCTGTGGAAAGTCCACTTCTTTGCGCATGCTGGCTGGTCTTGAGCCTATCGACGAGGGA
CGTCTACTCATTGATGGTAAAGACGCCACGGAACTGCGTCCGCAGGATCGTGACATCGCT
ATGGTCTTCCAGAGCTACGCGCTGTACCCGAATATGACTGTTCGGGACAACATGGGCTTT
GCGCTGAAGAATCAGAAGGTGGCTAAGGCTGAGATCGAAAAGCGTGTTGCTGAAGCCTCA
CGCATTCTGCAGCTGGATCCGTATCTTGATCGTAAGCCTGCAGCTTTGTCTGGTGGTCAG
CGCCAGCGCGTGGCCATGGGCCGTGCAATTGTGCGTGAGCCATCGGTGTTCTGCATGGAT
GAGCCACTGTCCAACCTAGATGCGAAGCTGCGTGTGTCTACGCGTGCGGAGATCTCTGGT
TTGCAGCGTCGCATGGGCGTGACCACGGTGTATGTGACTCACGATCAGGTCGAGGCCATG
ACCATGGGTGATCGCGTCGGTGTGCTTTTGCTCGGTGTGCTGCAGCAAGTAGACACCCCG
CAGAACCTGTACGACTACCCAGCAAATGCGTTCGTCGCCAGCTTGATTGGTTCCCCTTCC
ATGAACTTGATTGAGGGCACCATCCGTGGCGATAAGGTCACTTTGGGTACTGGAATTCAG
ATTTCAGTTCCTGATGAGGTGGCAGCAGAGGTTCGCAACAACCCGGATCGGTTTGAGGGT
CGTCCAGTCATTGTTGGTGCTGGTGCGGAGCACATGTATTTGACCACGGCGAATGAGAGT
GGTGCTGTATTGGGCGAAGTCAGGGACATTGATGAGCTCGGCGCGGATTCAATGGTCTAC
GTATTGGCGTGTGGTGTGAAGAACGCGAATACTGATCTTTTGGGTGAGGGGATTCCAGAG
GATATGCGCGTGACCGTTGTCGGTGCTGAAGAGACCGATAAGGCCCGGCTGGGTATTCGT
GTTGAGCGCCATCACGGTCTGAAGGCCGGCGATAAGGTGCACGTTGTTGCTGCACCGAAG
GATGTTCACCTCTTCGACGGTCTTGATGGCCGTCGAATGGGTGCATCGGTTGTAGCTCCA
GCCCATACAGTCCAGTCTGGTCAC
>RXN03129-downstream
TAGATTATTTACCAGTGCAACTC
>RXN03164-upstream
CTTTTTTGCATCCAGATGCACAAAGCCGTGGCAGAAACGAGACAAACTGAGCACAATGGC
TGTCATGGCATATCAACCAGCAGACAATCGCTATGACGAC
>RXN03164
ATGATCTACCGCAGGGTGGGAAATTCTGGGCTGAAGCTTCCGGCAATTTCGCTTGGGGTG
TGGCACAACTTCGGTGATGACAAGCCGCTTTCAACGCAGCGCAGCATTATTCACCGCGCG
TTTGATAGGGGAGTCACTCACTTCGATTTGGCTAATAACTATGGACCTCCAGCAGGTTCC
GCAGAGACCAACTTTGGCAGGATTTTGCGTGAGGATCTCAAAAGCCACCGCGATGAGTTG
ATCATTTCTTCCAAGGGGGGTTGGGATATGTGGGCTGGACCTTATGGTTTTGGTGGTTCG
CGAAAGTATCTAGTGAGTTCCGTTGATCAGTCCCTGACTCGCCTCGGCTTGGATTACGTG
GATATTTTCTATCATCACCGCCCGGATCCAGATACTCCTTTGGAAGAAACCATGTACGCA
TTGCGTGACATTGTTGCGTCTGGAAAGGCTCTTTACGTGGGTATTTCTTCCTACGGTCCA
GAGCTCACAGCGGAGGCGGCTGAGTTGATGGCGGAGGAGGGCTGCCCGCTTCTGATTCAT
CAGCCAAGCTATTCCATCATTAATCGTTGGGTGGAGGAACCGGGCGATGACGGTGAGAAC
TTGTTGCAGTCAGCTGCCAACAATGGTCTTGGCGTCATTGCTTTCTCACCACTTGCGCAG
GGCCTGCTCACGGACAAATATCTCGATGGAATTCCAGAGGGTTCCCGCGCCAGCCAGGGT
AAGTCCCTGTCTGAGGGCATGTTGAACGTGAACAATATTGATATGGTCCGCAAGCTCAAT
GACATCGCCCAGGAACGCGGGCAGTCACTTGCGGAGATGGCGCTTGCATGGGTGCTGCGC
GAGCAAAGAGAGTACGGCGCCGGATTACCG
>RXN03164-downstream
TGACCAGTGCATTGATTGGTGCT
RXS00088-coding Region
ATCGAAGACAACCACGGGACCGAAGGGATCTCCCTGCCAATCGAGGGCGTCGCTGCGACCGACAACCGC
GCATTCGAACTGCTTGATCGCTGGGGTGTAGAGCTCGTTGCAGCTCCACTTCAGCTGGTTCCATTTACC
GTTACGGGCTACACCGAAGAGGGCGGGGTCGCTAACCTTGGCTCCCACGGCGAGCCAGACCTGGAAGCA
CTTGCTGCTGCACAGCCTTGCCTGATCATCAACGGCCAGCGCTTGGCTCAGTACTACGATGACATCATT
GCCCTGAACCCTGACGCAACCGTTGTTGAGCTAGACCCACGCGATGGCGAGCCACTTGACGAGGAGCTT
ATCCGGCAGGCTGAAACCCTCGGTGAGATCTTCGGGGAAGAAGAAGATGCTGCAAAGATCGTTGCTGAT
TTCGAGTCCGCACTTGAGCGCGCTAAGACCGCATACGCAGCAATCTCCGACCAGACCGTCATGGCAGTT
AACGTTTCCGGCGGAAACATTGGCTACATCGCTGCTTCCGTTGGACGCACCTACGGTCCAATCTTCGAC
CTGGTTGGACTCACCCCAGCACTCGAGGTTGGCAACGCGTCCTCCGACCAGGAGGGCGACGACATTAAC
GTCGAAGCAATCGCAGCTGCAAACCCAGACCTGATCCTGGTCATGGACGGCGATGGTGGCACCAGCACC
CGCAACGAAGCTGATTACGTTCCAGCAGAGCAGATGGTCTCCGACAATGAAGCACTGGGAAACGTCAAG
GCTGTCACCGACGGATACGTTTACTACGCACCTGCAGATACCTACACCAAGGAAAACATCATCACCTAC
ACCGAGATCCTCAACGGCATGGCAGATATGTTCGAGAAGGCAGCTCAG
RXS00088-3′-Region
TAGGGGATCGATCCCACACTGAC
RXS00372-5′-Region
GCAGACATTTCCATAAGTCGTGCGAAATGCGCGCATTCATGTAAAGATGTTATTTCCTCCCCCAAACAC
TCCTTAAAATTTCAAGAAGGGCCTTATTTTC
RXS00372-coding Region
ATGTCTTCGAAGCACCCTTTGAAGCGGAGTGCCGTTACTGTTTTTGGACTCGGCGCTTGCGCTGCTCTC
CTCGTGGCTTGCTCTGAAGCTTCTGAGGACGTTTCCAGCGCAGAGACCACCACTGCAAGCTCTTCCGCT
AACGCATCCGATGCAGCCGGTGAAAAAGTAACGATCACCGTCTACACCTCTGAGCGTGAGGAAAAGGTC
GATGAGATCAACAAGGCGTTCATGGAAGCCAACCCAGATATTGAGGTTGAGGTGTAGCGCGCTGGTACT
GGCGATCTGAGTGCTCGCATTGAAGCTGAAAAGGCATCCGGTTCTATCGAGGCTGATGTGTTGTGGGCT
GCGGATGGTGCAACCTTTGAAACTTATGCAGCACAGGGCGACCTTGCAGAGCTGGAAGATGTTGAGACT
TCCGACATGATTGAAGAGGCTCTGGATGCTGAGAACTTTTATGTAGGCACCCGCATCATCCCAACCGTG
ATTGCATACAACACTGAAGTTGTTGATCAGGCTGAGCTTCCTACGTCTTGGGCTGATCTGACTGATCCT
AAGTATGCAGGCGAACTGGTCATGCCGGATCCAGCTGTGTCTGGTGCTGCAGCCTTCAATGCTTCTGTG
TGGAAGAACGACCCTGCGCTTGGCGAAGCCTGGATCACCGCCTTGGGTGAAAACCAACCAATGATCGCT
CAGTCCAACGGCCCAACCTCCCAGGAGATCGCTGGCGGTGGCCACCCAGTGGGCATCGTGGTGGACTAC
TTGGTGCGCGACTTGGCTGCTGGTGGATCTCCAATCGACACCATCTACGCATCGGAGGGTTCTCCTTAC
ATCACTGAGCCTGCAGGTGTGTTCGCTGATTCTGAAAAGAAGGAAGCAGCCGAGCGCTACATCAACTTC
CTGCTGTGTGTTGAAGGGCAGGAAATCGCAGTTGAGCAGGCATACCTGCCAGTGCGTGAAGATGTCGGA
ACTCCAGAGGGCACCCCCGAGTTGGCTGACATCGAGCTCATGACCCCTGACCTGGAGGTTGTAACCGCT
GATAAGGGGGCTGCTGTTGAGTTCTTCGAAAACGCAATGAAC
RXS00372-3′-Region
TAGTTTTCCTATGCAGTTATCTC
RXS00453-5′-Region
TAGTGGGGCGTGAAAAAATAGCTGATTTAAGAGGAGAAGCAACCCCGTGGCGAAATTGCTATTCAGGTT
GGGGCGATGGTCCTATAATCGCAAGTGGATT
RXS00453-coding Region
GTGATTTCGGCATGGCTAGTTATTTTGGCCATTGTTGGTGGTCTGGCCCTGACGATGCAGAAGGGGTTG
AGTAACTCTTTCACTATTGAAGACACCCCTTCGATTGATGCCAGTGTTTCTCTGGTTGAAAATTTCCCT
GATCAGACGAACCCGGTGACGGCCGCGGGAGTTAACGTGGTTTTCCAATCCCCGGAAGGAACCACGCTT
GATGATCCTCAGATGATGACTGCGATGGATGCAGTCGTTGATTACATTGAGGACAATTTGCCTGATTTT
GGTGGGGGAGAGCGCTTCGGCAATCCTGTTGAGGTGTCTCCTGCGTTGGAAGAGATGGTCATCGAGCAG
ATGACCAGCATGGGGCTTCCTGAGGAAACCGCTGCAAAGGATGCTGCCAATCTGGCGGTGTTGAGCGAA
GACAAAACCATTGGCTACACCTCTTTCAACATTGATGTTGAGGCCGCAGAATATGTGGAGCAAAAACAC
CGCGATGTGATCAACGAAGCGATGCAAATCGGTGAAGATTTAGGTGTCCGGGTGGAAGCCGGTGGACCT
GCTTTCGGTGATCCAATTCAGATTGAAACCACCAGTGAGATCATCGGTATTGGCATCGCGTTCATCGTG
TTGATTTTCACCTTTGGTTCTTTGATTGCTGCAGGCTTGCCTTTGATTACCGCGGTGATCGGCGTGGGC
ATTGGTGCGCTGGCAATTGTGCTGGCCACGGCGTTTACTGATCTCAACAATGTGACTCCAGTGCTCGCA
GTGATGATTGGCCTGGCCGTGGGCATTGACTACGCGCTGTTTATTTTGTCTAGGTACCGTGCGGAGTAT
AAGCGCATGCCACGTGCCGATGCTGCCGGAATGGCGGTGGGCACAGCTGGTAGTGCGGTGGTGTTTGCT
GGCGCGACGGTGATTATCGCGCTGGTAGCCCTCATCATTGCGGATATCGGATTCCTCACGGCCATGGGT
ATTTCTGCGGCGTTTACGGTGTTCGTGGCTGTGCTCATTGCGTTGACGTTTATCCCGGCGCTGTTGGGT
GTGTTTGGTGGTCATGCGTTCAAGGGCAAGATCCCTGGAATTGGTGGAAACCCAACGCCAAAGCAGACG
TGGGAGCAAGCGCTTAATCGTCGTTCGAAGGGTCGCTCATGGGTCAAGCTTGTACAGAAAGCACCGGGT
CTTGTGGTGGCAGTGGTGGTCTTGGGTCTTGGTGCCTTGAGCATTGCTGCAATGAACCTGCAGTTGTCA
CTGCGTTCTGACTCCACCTCCAATATTGATACCACTGAGCGTCAGTCGGCTGATTTGATGGCAGAGGGC
TTTGGCGCGGGCGTTAATGCGCCGTTCTTGGTCATCGTCGATACGCATGAGGTCAATGCTGATTCCACC
GCATTGCAGCCACTGATTGAGGCACAGGAGCCTGAAGAGGGCGAGTTCGATCGGGAGGAGGCGGCTGGT
TTTGCTACCTATATGTATGTCACGCAGAGCTACAATTCCAACATCGATGTGAAGAATGCGCAGATCATC
AGCGTCAATGATGATTTCACTGCGGCGCAGATTCTCGTGACTCCATACACCGGACCTGCGGATAAAGAG
ACCCCTGAGTTGATGCACGTGCTGCGTGCGCAGGAAGCTCAGATTGAGGATGTTACGGGAACTGAACTG
GGTACCACTGGGTTTACGGCGGTTCAGTTGGACATTACTGAGCAGCTGGAAGACGCAATGCCGGTTTAC
CTCGCTGTGGTTGTTGGTTTGGCTATTTTCCTCCTCATTCTGGTGTTCCGTTCCCTGCTTGTTCCGCTG
GTTGCTGGCCTTGGCTTCTTGTTGTCTGTGGGTGCGGCCTTCGGTGCGACGGTGTTGGTCTGGCAGGAG
GGCTTCGGTGGCTTTGTGAACACCCCTGGTCCGCTGATTTCCTTCATGCCGATCTTCCTCATCGGCGTG
ACCTTCGGTTTGGCCATGGACTATCAGGTGTTCCTTGTGACTCGCATGCGCGAGCACTACACCCACCAC
AATGGCAAGGGACAGCCTGGTTCCAAGTACACCCCGGTTGAGCAGTCAGTGATTGAAGGCTTCACGCAG
GGCTCCCGCGTGGTTACAGCAGCGGCACTGATCATGATTGCCGTGTTCGTGGCGTTTATTGATCAGCCG
TTGCCATTTATTAAGATCTTCGGTTTCGCGTTGGGTGCGGGCGTGTTTTTCGATGCTTTCTTCATTCGC
ATGGGTCTGGTCCCCGCGTCGATGTTCCTGATGGGCAAGGCCACGTGGTGGATGCCTAAGTGGCTGGAT
CGAATTCTGCCAAGTTTGGACATTGAAGGCACCGCACTGGAGAAGGAATGGGAGGAGAAGCAGGCTGCA
CGT
RXS00453-3′-Region
TAGACTTGGCACCTATGTCAGAT
RXS00479-5′-Region
TAGATCCCAAGGCTCAAAATTTATTACTTAAACAAGTTGAGCAACTAGCCAGCCGCAAATCTTAGAACT
AACCTTTACGCCTTTAACGGAAGTGAATTTG
RXS00479-coding Region
ATGTCTACTAGCATCACAACAGAGAACAAGAAGAAATCTGGTCCTCCTCGCTTGATGAGAATCTTTCTG
CCCGCCTTGCTAATTTTAGTTTGGCTTGTAGGAGCTGGAGTCGGCGGTCCTTATTTTGGCAAGGTTAGT
GAGGTCTCCTCCAACAGCCAGACCACATATCTGCCAGAATCTGCCGATGCCACTCAAGTACAGGAACAG
TTGGGAGATTTTACTGATTCTGAATCCATCCCAGCCATTGTCGTAATGGTCAGCGATGAACCCTTAACA
CAGCAAGACATCACACAACTCAATGAAGTTGTTGCTGGGCTTTCAGAATTAGACATAGTTTCCGATGAA
GTCTCCCCTGCTATTCCATCCGAGGACGGCAGAGCTGTCCAAGTGTTTGTCCCCCTCAATCCATCAGCG
GAGCTGACGGAAAGCGTCGAGAAGCTCTCTGAGACCTTGACCCAGCAAACGCCGGACTATGTGAGCAGC
TATGTGACCGGACCGGCTGGGTTTACCGCTGATCTCAGCGCAGCTTTCGCGGGTATTGATGGGCTAGTC
CTAGCAGTCGCCTTGGCTGCCGTCCTTGTCATTCTTGTCATCGTCTATCGCTCCTTCATTCTGCCCATC
GCCGTGCTTGCCACCAGTTTGTTTGCGCTGACTGTAGCTCTATTGGTGGTGTGGTGGCTAGCTAAGTGG
GACATCCTGCTGCTTTCGGGTCAGACTCAAGGCATCCTCTTCATTCTGGTCATTGGCGCCGCCAGCGAC
TACTCATTGCTATACGTTGCTCGTTTCCGTGAAGAGTTACGCGTTCAACAAGATAAAGGGATAGCCACA
GGGAAAGCCATCCGGGCATCGGTGGAACCCATTCTTGCCTCGGGCAGCACTGTTATTGCGGGCCTCCTT
TGTTTGCTATTTAGTGATTTGAAATCTAACTCCACGCTAGGTCCAGTAGCTTCGGTGGGCATTATTTTT
GCAATGGTTTGTGCTCTTACTCTGCTACCAGCCCTGCTGTTTGTATTCGGTCGGGTGGCCTTTTGGCCC
AAGCGACCAAAATACGAACCTGAAAAAGCGCGTGCGAAAAACGACATCCCCGCCAGCGGGATCTGGTCA
AAAGTGGCTGATTTAGTGGAGCAGCATCCTCGTGCAATCTGGGTATGTACACTTATTGTGGTTCTCTTG
GGTGCGGCTTTCGTTCCCACACTAAAAGCGGACGGTGTGTCCCAATCCGACCTAGTTCTGGGTTCCTCT
GAAGCACGTGATGGCCAGCAGGCTTTAGGCGAACACTTCCCCGGTGGATCCGGCAGTCCTGGTTATATT
ATCGTTGATGAAACACAGGCAGCACAGGCTGCTGACGTAGTCCTTAACAACGACAATTTCGAGACTGTA
ACTGTAACTAGTGCTGAGTCCCGCTCTGGCTCAGCCCCAATCACCGCTGACGGTATTGTGCCGTTAGGT
TCTGGTACAGCTCCAGGCCCGGTAGTTGTAGAAGGGCAAGTCCTTTTACAAGCAACACTTGTCGAAGCA
CCAGATTCCGAAGAAGCTCAAAAAGCTATTCGCAGTATCCGCCAAACTTTTGCAGATGAAAATATATCA
GCGGTAGTAGGCGGTGTCACTGCAACTTCCGTAGACACTAACGATGCCTCCATCCATGACCGCAACCTG
ATCATCCCAATTGTATTGCTGGTCATTTTGGTTATTCTCATGCTGTTGCTGCGGTCTATTGTCGCACCA
CTCCTGCTAGTAGTCACCACCGTGGTGTCTTTTGCTACTGCTTTAGGCGTGGCTGCTTTACTTTTCAAT
CACGTTTTCAGTTTCCCAGGAGCAGACCCCGCAGTACCTCTCTACGGATTTGTATTTTTAGTAGCCTTG
GGCATCGACTACAACATTTTCTTAGTCACCCGAATCCGTGAAGAAACCAAAACCCACGGCACAAGACTT
GGAATTCTTCGAGGCCTGACAGTAACCGGCGGAGTAATTACCTCAGCTGGAGTAGTTCTCGCCGCAACG
TTCGCAGCACTCTATGTCATCCCAATTCTATTCCTGGCACAAATTGGCTTCATTGTCGCTTTTGGAGTT
CTTATTGATACCCTGCTCGTTCGCGCCTTCTTGGTGCCTGCTTTGTTCTACGACATCGGACCGAAAATC
TGGTGGCCGTCAAAATTGTCCAATCAGAAATACCAGAAGCAGCCTCAGCTA
RXS00479-3′-Region
TGACACACCAAAATTCGGCTGTC
RXS00654-5′-Region
CAGCAATAGCGATTATTGCTTGATTGTGTGTTTTTAGATCTTCGGTTCTCTTCACTCAACTGCTGTGAA
GTGCCACCTGTTTGGAAAGGCGAACACGATA
RXS00654-coding Region
GTGCTCGATATTTTGATTTACCCGGTGTCTGGAGTGATGAAGCTGTGGCACCTGCTTCTTCACAACGTT
GCGGGTTTGGACGATTCACTGGCGTGGTTCTTTTCCCTTTTCGGCCTTGTCATCACGATCCGTGCAATT
ATCGCGCCTTTCACCTGGCAGATGTATAAGTCGGGCCGCACTGCCGCACATATTCGTCCTCACCGCGCT
GCGCTCCGGGAAGAATACAAGGGAAAGTACGATGAAGCGTCCATTCGGGAGTTGCAGAAGCGCCAGAAT
GATTTGAATAAGGAATACGGCATTAACCCGCTGGCAGGTTGTGTGCCTGGGCTGATCCAGATACCGATT
GTCCTTGGTCTTTACTGGGCACTTCTCCGCATGGCTCGCCCTGAAGGTGGTTTGGAAAATCCCGTCTTC
CAGTCGATCGGCTTCCTAACTCCTGAGGAAGTGGAATCTTTCCTCGCTGGTCGCGTGAGCAATGTGCCT
CTGCCCGCTTATGTTTCGATGCCCACTGAGCAGCTAAAATATTTGAGCACCACGCAGGCGGAAGTTCTT
AGTTTCGTTTTGCCACTGTTCATCACAGCCGCAATCCTCACCGCAATCAACATGGCGATGTCCATGTAC
CGCAGCTTCCAAACCAACGATTACGCATCCGGATTCTCTAACGGCATGCTGAAGTTCATGATCGTGATG
TCGATCCTCGCGCCGATCTTCCCACTGTCCCTTGGCCTCACAGGAGCATTCCCCACAGCAATCGCACTC
TATTGGGTGAGCAACAAGCTGTGGACGCTCCTCCAAACAATCATCATGATGGTCATTTTGGAACGCAAA
TACCCACTTACCGACGATTTCAAAGTGCACCACCTAGAGCAGCGCGACATCTACCGCGCAAAACAAAAA
GAAAAGCGCATCTTGCTGTGGACACGACGCAAAAACCGCGCCCTGATGATTCTCACCGCATGGAACGCC
TCAACGCTTCACGCAACAAACGTGGAACTCACGAAAAGCCGTACTGCCGAAATCAACGAAGCAAAACAG
GCCCGCAAAGAAATCGCGAACAAGAGGCGCGAAACGCAACGTGAAATGAACCGCGGCGCCATGCAGCGC
TTAAAGCAGCGTCGGGCTGAGGTTAAAGCTAAAAAGAAGGGGCTTATCGACGCCTCCCCCAACGAAGAT
ACCCCTTCGGAAAATGAAGAAACTAAATTGAGTAGTCCGCAGGTGGAGCCGACAACAACTGCCGAGCCA
AATCGCGAGCCGTCTCAAGAGGAC
RXS00654-3′-Region
TGATGTTGTGGACCAATCGAGAT
RXS0075B-5′-Region
TTCAAGTTTGGCTGTGACTCATGTCGCACATAGTATTTCAATCACCGGATCCGCAGGATTGCAAAATGC
TGGGGAATATTCATAACAACGGAGGTCAGTC
RXS00758-coding Region
ATGACTTTGAAGAAGTCTCTCGCTGTAACCACGGCGGCTGCACTTGCTTTGAGCCTTGCCGCTTGGTCG
TCCGACTCCTCGTCCGACAGCTGCTCATCCTCATCAGGCAGCGAAGGCGGCGACAACTACGTCCTCGTC
AACGGCACTGAGCCAGAGAACGCGCTCGTCCCAGGCAACACCAACGAAGTAGGTGGCGGTCGCATGGTC
GACAGCATCTTCTCCGGCCTGGTCTACTACGACGTCGAGGGCTCCCCTGTCAACGATGTTGCAGAGTCC
ATCGAACTCGAAGGTGACAAGACCTACCGCATCACCATCAAAGACGGCCAGACCTTCACCGATGGCACC
CCAGTTACGGCTGAGAGCTTTGTCAACGCATGGAACTACAACGTAGCTAACAGCACGCTGTCCTCCTAC
TTCTTTGAGTCCATCGTCGGCTACGAAGAAGGCGTCGAGTCCATGGAAGGCCTCCAGGTCGTCGACGAC
ACCACCTTCACCGTCGAGCTCACCCAGCCTGAGTCCGACTTCCCACTGCGCCTGGGATACTCCGCATTC
TTCCCGCTTCCTGAATCCGCATTTGACGACATGGACGCATTCGGTGAGAACCCAATCGGCAACGGTCCA
TACAAGCTCCAAGAGTGGAACCACAACCAGGACGCCACCATCGTTCCTAACGCGGACTACACCGGTGGA
CGCCAGGCTCAGAACGACGGCGTGAAGTTCATCTTCTACCCAACCTTCGACTCCGCTTACGCGGACCTG
CTCTCCGACAACTTGGATGTGCTGGACGCTATCCCAGACTCCGCGTTCTCCTCCTTCGAGGACGAGCTC
TCTGGCCGTTCCATCAACCAGCCTTCCGCTGTGTTCCAGTCCTTCACGATCCCGGAGAGCCTTGAGCAC
TTCTCCGGCGAAGAAGGCGTGCTGCGTCGCCAGGCCATCTCCTTGGCCGTCAACCGCGACGAGATCACC
CAAACCATCTTCGAAGGGACCCGCACCCCAGCGACGGACTTCACCTCCCCTGTCATCGACGGACACTCT
GATTCCCTCCAGGGCGCAGATGTCTTGACCTACGATCCAGAGCGCGCTCAGGAACTGTGGGCACAGGCA
GACGAGATCAGCCCTTGGTCCGGCGAGTTCTCCATCTCCTACAACGCAGACGGTGGACACCAGGCATGG
GTGGACGCAACCGCCAATTCCATCCGCAACACCCTGGGTATCGACGCCATCGGCAACCCATACCCAGAC
TTCAAGTCCCTGCGTGACGATGTCACCAACCGCACCATCAACGGCGCATTCCGCACCGGCTGGCAGGCA
GACTACCCGTCCTTGGGGAACTTCCTCGGACCTTTGTACGGCACCGGTGCAGGCTCCAACGATGGTGAC
TACTCCAACCCAGATTTCGATGCCAAGGTCGCCGAAGCAGCAAACGCGGCCGATGTTGACGCATCAACC
CCGCTATACAACGAAGCACAGGAAATCCTGCTCCAGGATCTGCCAGCGATCCCAACTTGGTACTCCAAC
GCAGTTGGTGGATACTCCACCAACGTGGACAACGTGGAATTCCAGTGGAACTCGCAACCTGCGTACTAC
CAGATCACCAAGAAC
RXS00758-3′-Region
TAGTAGCTTCGGACCACCCGCTC
RXS00912-5′-Region
CCACACCTTTGAAAGGAGCTAAGGG
RXS00912-coding Region
ATGGACAACAGCGTCTACACAGCAGGCCTCACAATCGCAGCTGCCTTTTTCATGCTGTCGTTCATCTTC
ACCATCTACCGCATCATCGTCGGGCCCAACTCCATCGATCGCCTACTCGGCCTGGACGGAACCGTCTCC
ATGATTCAATGCTCCATGGCCACCTACATCTGCTGGACACTCGACACCACCGTCACCAACTTCATGATG
GTCATCGCACTCTTAGGATTCATCAGCTCTGTATCCGTAGCCCGCTTCCGCAAGAGGGATGGTGCC
RXS00912-3′-Region
TAAATGACCCTGCAACTATTCAC
RXS00932-5′-Region
CCCAATTAATTTATGCACTTCGGTGAGGTTACTCACAAAGAGTAGCGTGCAAAGCCCAGCAATAAGGTG
ATGTTTCAACGATTAGGTTACGGTAGGGGCC
RXS00932-coding Region
ATGACGGCACAGAAACTTCACCGTTTTGCAGCCCTTTTAGAAATGGGTACCTGGACCCTGCTGATCATC
GGCATGATCTTAAAATACAGTGGAGTGACAGACGCCGTAACCCGTATTGCCGGCGGTATCCACGGGTTT
GGCTTGCTCTGTTTTGCAGCCATCACCATCACCGTGTGGATCAATAATAAGTGGACATTCCCGCAGGGT
ATCGCAGGTTTGATCGTCTCTGTTATCGCGTGGGGTGCATTGCCATTTGCATTGTGGGGAGACAAGAAG
GGCCTCGTTGGCGGCGGATGGCGCTTTTCAGATCGGTGGGAAAAGCCACACACTTTCTTTGACAAGATC
TTGGCTCAATTGGTCAGGCACCCAATCGGATCCATTTTAATTCTGCTGGTGATTATCGCCGTCGTCTTG
TGTATGTTGCTGGCGATGGGACCACCTTATGATCCAGATGCCATCGCAAACACTGTGGAT
RXS00932-3′-Region
TAAACAACAGCCTCCTTCACATG
RXS01346-5′-Region
AAGGTGTGGTGAGTCACTGGCTAGATTTGATTTGTTGGCCATACCAAATCGGCCCACACAGGCACGTTG
CAAACAGGAACGCTCACCCATAGGAGATTTA
RXS01346-coding Region
ATGCGCACAGCCACAAAAGTCATCGCAACAGTGATGGCCTCAACCCTGGCTATCGGGCTGGCATCTTGT
TCCAGCTCTAGTGGCACCGCAGACGTGAATTAGGTATCCGTCAACGGCACCGAACCTCAGCGCGGACTC
ATCCCGGGCGACACCAATGAAAACGGCGGTGGGCGAGTGGTGGACATGCTGTACTCTGGGCTCGTCTAC
TTTGATGAAGCTGGCGTTGCTCAAAATGACCTGGCGGCATCAATTGACCAGGAAACAGACACCACCTAC
AAAATCACTTTGCGTGATGGCATCAAATTCAGTGACGGATCGGATATTACTGCGAGTGATTTTGTGGAT
ACCTGGAATTTTGTAGTGGAAAATGGACTGCTCAACACTTCTTTCTTCTCACCGATTAAAGGGTATGAG
GAGGGCGTGGAAACGCTCGAGGGTTTGAATGTGGTGGATGATCGCACATTTACCATCGAGCTTGCCCAA
CCGGATTCTGAGTTCACCCAACGCATTGGCTACTACGGTTTTGCACCGATGCCAGCTTCGGCTCGCGAT
GATATTGACGCCTTTGGTGAAAACCCCGTGTCCTCTGGCCCTTACAAACTAGAGCAGTGGGATCACAAC
GCAGAACTGAAAGTGGTGGCCAATGAACACTACGATGGCCCGCGCGCAGCCAACAACGATGGGTTGAAG
TACGTGTTCTACGCCGAAAATGATGCAGCTTATTCAGATCTGTTGGCTGGAAACCTAGATGTGCTGGAT
CTCATTCCACCATCGGCGTACACCACCTATGAAGAGGAACTGTCGGGTCGATCCATTAATCAACCTGCG
GCCTCCTATCTGGAACTCTCCATTCGCATGGAATCCGCCAACTTTGAAGGGCAACAGGGACAGTTGCGT
CGACAAGCAATTTCTATGGCGATTAACCGTGAAGAAATCGCTGAGCAGATCTTCGCCGGCACCTACACG
CCTGCGCTCGACTTCACCGCGCCCGTGCTCGACGGCTGGGGCGATGATTTGAACGGCAATGACGTGCTG
ACTTTCCAGCCTGACAAGGCCCGTGAGCTGTGGGAAGACGCTGAGGAGATCGCACCTTTTGAGGGCGAA
TTGCAGATCAGTTACAACGCGGATGTTCCCAACCGGGAATGGGTGGATGCGGTAGCAAACAGGATCAGC
AACGAATTAGACGTCAACGCCACTGGCAATCCTTTCCCCGATTTTAAATCCTTCCGCGACACATACCGC
ACCACCGGATTGGATGGGGGCTACCGCACCGCGTGGTTTGCGGACTACCCAAGCATCGGCAACTTCCTT
GGACCTAACTACACCTCGGGCGTGGCCTCCAACGATGCCAAGTACGAAAACCCAGAATTTGATCAATTG
ATTGCCGACGCCGCAGCAGCCTCCACCAAGGAGGAAACCTTCCAGGCATATGCGCAGGCCCAGGAAATG
TTGTTGCGCGATCTTCCCGCAATCCCACTGTGGTACCCGAATGTGGTTGGCGGCTACTCAGAATCCGTG
GACAACGTCTCCGTAAACTGGAAGGCCATACCTGTTTATTGGGCAATTACAAAGCAA
RXS01346-3′-Region
TAAAGTCATTAACCTAAATCCGG
RXS01425-5′-Region
AGTCCCTATTAATCCCAAGGAGTTTCGACTCACAGTGCTCAATTTCATTTATTGGCCAATTTGGGCCAT
TCTGTGGTTCTGGCATAAAGCGTTCAGCTTT
RXS01425-coding Region
GTGCTGAGCCCAGATTCCGGAATTACCTGGGCCTTGTCGATGATGTTCTTGACCTTCACCGTGGGTATG
GTTCTGGTCAAGCCGATGGTCAACACCATGCGTTCACAGCGCAAGATGCAAGACATGGCTCCAAAGATG
CAGGCCATCCGCGAGAAGTACAAAAATGACCAGCAGAAGATGATGGAGGAGACCCGCAAACTTCAAAAA
GAAGTGGGCGTTAACCCCATCGCAGGCTGTTTGCCAATGTTGGTGCAGATCCCAGTGTTCCTGGGTCTG
TTCCACGTGCTGCGCTCCTTCAACCGCACCGGTTCTGGCGTTGGCCAGCTGGAAATGACCGTTGAGCAA
AACGCGAACACCCCGAACTACATCTTCGGTGTCGACGAGGTTCAGTCCTTCCTGCGTGCAGACCTGTTC
GGTGCGCCACTGTCGTCCTACATCACCATGCCTGCTGACGCGTTCGACGCGTTCCTTGGCCTGGATGTC
TCCCGCCTCAACATCGCGCTGGTTGCAGCTCCAATGATTTTGATCATTGTCGTGGCAACTCACATGAAC
GCGCGTCTGTCCGTCAACCGCCAGGAAGCTCGCAAGGCAGCCGGCAAGCAGCAGGCCGCTTCCAGCGAT
CAGATGGCCATGCAGATGCAAATGATGAACAAGATGATGCTCTGGTTCATGCCAGCCACCATTTTGTTC
ACCGGCTTCATCTGGACCATCGGTCTTCTTGTCTACATGATGTCGAACAACGTGTGGAGCTTCTTCCAG
CAGCGCTACATCTTCGCCAAGATGGACGCTGAGGAAGCAGCTGAGGAGGAGGAAAAGCGCGCAGCAAAG
CGCACTACCGCTCCAAAGCCTGGCGTGAAGCCAGAAAACCCCAAGAAGCGTAAGAAG
RXS01425-3′-Region
TAAAACTTCACTAAAAAGCGCCA
RXS01658-coding Region
GATCCACAGATCCTGTCACCAACCTTCACCCAGCAACAGCAGCTGCGAAACTTCTACGGTTTCCCAGAC
CAGCTGGCGATGGACCGCTTTGAAGTAGATGGCAAACTCCGCGACTTTGTTGTGGCAGCACGTGAGCTC
GATCCAAACGCCCTGCAGCAAAACCAGCAGGACTGGATTAACCGTCAGACTGTTTATACCCACGGCAAC
GGCTTCATTGCAGCTCAAGCAAACCAGGTGGATGAGGTCGCCCGCGACGTCGGATCCACTCGTGGTGGT
TACCCTGTCTACACCGTCTCTGATTTGCAGTCGAATGCTGGTGCTGGAGAAAGCGAAGATGCTGAGGAG
CTTGGCATCAAGGTTGATGAGGCTCGTGTGTACTACGGACCACTGATTGCTTCTGCGACTGATGGTGCT
GACTACGCAATTGTCGGTGACACCGGCGATGGCCCAGTCGAGTAGGACACTGACACCTCCAGCTACACC
TACGAAGGTGCTGGCGGCGTGGACATTGGAAACATGGTGAACCGTGCGATGTTTGCATTGCGCTACCAG
GAAATGAACATGCTCCTGTCTGATCGTGTTGGTTCCGAATCCAAGATCCTATTTGAGCGCGATCCTCGT
TCCCGTGTGGAAAAGGTTGCACCTTGGTTGACCACTGACTCCAAGAGCTACCCAACTGTGATTGATGGT
CGCATCAAGTGGATCGTCGATGGCTACACCACCTTGGATAGTCTTCCGTACTCCACGCGCACCTGACTG
ACGGAAGCGACTCAGGATGCTGTCATGCCTGACGGCACCCGACAGCCACTGATCAGAGATAGGGTCGGT
TACATCCGCAACTCCGTGAAGGCTGTTGTTGATGCGTACGACGGAACTGTTGAACTCTACGAATTCGAC
ACCGAAGATCCTGTTCTGAAGGCATGGCGTGGCGTGTTCCCAGACACCGTGAAGGACGGGTCGGAGATT
TCCGATGAGCTTCGCGCACACCTGCGTTACCCAGAAGATTTGTTCAAGGTCCAGCGTGACATGCTGGCC
AAGTACAACGTTGATGATTCTGGAACATTCTTCACCAACGATGCGTTCTGGTCTGTCCCAGGTGACCCA
ACTGCAGGGGAGGGCCGCCAGGAACTTAAGCAGCCTCCTTACTACGTGGTGGCAGCAGACCCAGAGACC
GGTGAGTCCAGCTTCCAGCTGATCACCCCGTTCCGTGGACTTGAGGGCGAGTACCTCTCTGCACACATG
TCTGCGTCGTCTGATCCAGTTACCTACGGTGAAATCAGTGTTCGTGTGCTGCCTACGGATTCTGTGACC
CAGGGTCCAAAGGAGGCCCAGGATGCGATGATGTCATGTGACCAGGTTGCTCAGGACCAAACACTGTGG
CGTGGATCGAACGATCTGCACAACGGAAACCTGTTGACGTTGCCAGTTGGTGGCGGAGAGATCCTCTAC
GTTGAGCGGATTTACTCGCAGCGCAAGGATCAGGCATCGGCGTTGCCGAAGCTTCTGCGCGTGCTGGTC
TTCTACAAGGGTCAGGTTGGTTACGCACCAACGATCGCTGAAGCCCTATCGCAGGTCGGCATTGATCCG
AAGGAAGCGCAGGACATCGAAGAGGTAGATGGCACCGCTACGACGCCATCGACTGATGAGACTGACACT
GACACTGATCAGCCTGCAACCGAAACCCCAACTGCACCAGTGAGTGAGGCGGAAGGAATCGCGGCCATC
AACGATGCGTTGAGCAACCTTGAAGCTGCTCGCGATAGCTCTTTCGAAGAGTATGGTCGTGCACTCGAT
GCGCTTGATCGTGCCGTCGATAGCTACCAGTCCGCACAG
RXS01658-3′-Region
TAGCGTTTGAGTAAACAGCCCGA
RXS01677-5′-Region
GTCGGCATAGTTGAGTTTTATTCATGGCTTTTAGCTAGGCGACTTTAGTTGAGGGCTTTTAGTTGAGGG
CTTCCCAGCAGGGATGGTTAAGGAGAATTCA
RXS01G77-coding Region
GTGAACCAACAGAGTAAAAAGTGGCTCGTACCGACACTGGTCGTCATCATTGCAGTGCTCCTCATCGCA
GTTGTTCTGTTGATGTACCGAGGAAATGCGAGTGATACGGCCGAGGGCGTTTCAGCCGCTGCGACTTCG
GACTCGGCTGCTGCTTCGACTGCTGCTTGGGGTTCCGCTTCTGGTGGTGCGGACTCCGATCTGACCAGC
GTGGAAGCACGCGACCCTTCCGACCCTGTTGCGGTGGGAGACGTTGATGCACCTGTTGGGTTAGTGGTG
TTTTGCGACTAGCAATGCCCGTTCTGTGGAAAGTGGAGGGATGAAACGGTGCCACAGATGATGAAGCAT
GTGGAAGATGGAAACCTCCGCATTGAATGGCGTGAAGTGAACATCTTTGGAGAACCATCTGAGCGTGGA
GCTCGCGCGGCATACGCTGCGGGTTTGCAGGACGCATACTTGGAATACCACAACGCACTCTTTGCCAAC
GGTGAAAAACCCAGCGAAGACCTGCTCAGCGAAGAGGGACTTATTAAGCTTGCTGGTGACCTTGGACTA
GACGAATCGAAATTCACTGCCGATTTCCAATCCCCTGAAACTGCAGTCGCAATTGCGCAACATCAACAG
CTGGGAATCGATGTTGGCGCCTACTCCACCCCAGCTTTCCTCCTAGGTGGCCAGCCAATCATGGGCGCT
CAGCCTGCTTCTGTATTTGAAGCCGCCTTCGAGCAAGCACTGGCAGCGAAAGAA
RXS01677-3′-Region
TAAACCGTGGATGTCGGCCTAGT
RXS02586-5′-Region
TTCTCTGAGATCGTCATGATGAAGTACATCGGGTTCGGCATGATCGCAGCGCTGATTCTGGATGCCACC
ATCATCCGCATGCTGCTTGTCCCCCGCCGTG
RXS02586-coding Region
ATGCACCTGCTTCGCGACGACAACTGGTGGGCACCGGGCTTCGTTAAAAAGGCCTACACCGTCATGGGT
CACGGCTCTGAGGTGGAGGAAGCACCTCGCCCAACCAGCCGTCGCCTCAACGACGATGAGGAAGTCACC
GTGCATGAAGCAGTTGTCGCTGGCGATACCGTGGCATCTCGCGGTGGTTTGAGCACGCAGGAAAACCGT
GATCTGGTGTCCTTCGTGGAACTTAAGGCTCGTTTGGAAAAGCGCAGGCTTGAGGATCTAGAT
RXS02586-3′-Region
TAAATCTATGCGAGGATTTTTCA
RXS02587-5′-Region
AGCCTGGATAACCTGCGAGACGGTGGCGCATGGCTGCAGCCGTTCCGCCCTCTGACTGCCTTGTTATCC
AACCGCCACAATTCCCAGGAGTAATCCACCC
RXS02587-coding Region
GTGTTTTCTAAATGGGGCCACTTTGCTTACAGATTTAGGCGCATTGTTCCGTTAGTCGTCATCGCCGGG
ATTTTGGCTTTGTTTGTGATTTTCGGCACCAAGCTGGGCGACCGCATGAGCCAGGAAGGATGGGATGAT
CCTGGTTCTTCCTCGACCGCTGCGGCGCGCATCGAGTTGGAGACCTTTGGGCGTGACAATGAGGGCGAT
GTCGTGTTGCTGTTTAGTGCGCGTGAAGGCACTTCTTTCGATGATGCAGAGGTGTTCTCCAGCATCTCT
GGCTACTTAGATGGGCTAATCGAGAACAACCCTGATGAAGTCAGCCACATCAACAGCTACTTTGACACT
CGTAATGAAAATCTCCTCAGGAAAGACGGCACCCAAACCTTTGCAGCTCTCGGGCTGAAAGGTGACGGC
GAGCAAACGCTGAAGGACTTGCGGGAGATTGAAGATCAGCTCCATCCGGACAACCTTGCCGGTGGCGTC
ACCACTGAGGTCGCGGGTGCCACCGCTGTAGCCGACGCACTCGATGAGGGCATGGCTGGCGATATTTCA
CGCGCCGAAGTTTTTGCGCTGCCTTTCGTGGGTATGTTGCTGCTCATCGTGTTTGGCTCAGTTGTTGCC
GCGGCGATGCCATTGATCGTGGGCATTTTGTGCATCTTGGGTTCGCTGGGCATCTTGGCAATTTTGGCT
GGATTCTTCCAGGTCAAGGTATTTGCACAATGTGTTGTGACCCTTCTGGGCTTGGGTCTTGCCATTGAC
TATGGCTTATTCATGGTCTCTCGTTTCCGTGAGGAAATGGATAAGGGCACCCCGGTTGAACAGGCTGTT
GCGACGAGTACGGCGACCGCGGGTAAGACTGTGGTGTTGTCTGCAGCGATGGTGGCTGTGGCGCTGTCC
GGGTTGTTTGTTTTCCGACAGGCTTTCTTGAAGTCGGTGGCATTGGGTGCGATTTCGGCGGTTGGCCTT
GCTGCTTTGATGTCGGTGACGGTGTTGCCGTCGCTGTTCAGCATGTTGGGTAAGAATATCGATAAGTGG
AGTTTGCGTCGCACTGCTCGAACAGCGCGCCGTTTGGAAGACACCATTTGGTACCGCGTGCCGGCATGG
GCAATGCGCCATGCCAAGGCAGTGACCGTGGGCGTCGTATTGCTCTTGCTTGCTCTTACAGTGCGGTTG
ACGGGCGTGAAATTCGGCGGCATCAATGAAACGTATCTGCCACCAGCTAACGACACCCGCGTCGCCCAA
GAGCGTTTCGACGAGGCGTTTCCCGCCTTCCGCACCGAGCCGGTCAAGCTTGTGGTCACCGGGGCGGAC
AACAACCAGCTGATCGATATCTATGTTCAGGCCAACGAAGTTGAGGGACTGACAGATCGTTTCACCGCA
GGTGCGACTACCGATGATGGCACCACGGTGTTGTCTACTGGTATTCAGGATCGTTCCCTCAATGAGCAG
GTAGTGGAGCAGCTTCGCGCTATTTCCGTCCCTGAGGGCGTTGAGGTGCAGATCGGTGGCACTCCAGCC
ATGGAGATCGAATCCATTGAGGCGCTCTTTGAAAAGCTCCTCTGGATGGCTCTCTACATTGTGCTGGCC
ACTTTCATCCTCATGGCATTGGTATTTGGTTCGGTGATTTTGCCGGCGAAGGGCATCATCATGACCATT
CTGGGTATGGGTGCCACCTTGGGTATTCTCACCTTGATGTTCGTGGATGGCGTGGGTGCCAGCGCATTG
AACTTCTCCCCTGGCCCACTGATGAGTCCAGTGCTGGTGCTGATCATGGCTATTATTTACGGACTTTCC
ACCGACTATGAGGTGTTCCTGGTATCTCGCATGGTGGAGGCCCGCGATAAAGGCGAATCCACCGACGAC
GCCATCAGATACGGCACTGCACACACCGGATCTATGATCACCGCGGCCGCACTGATCATGATTGTGGTC
TGTGGAGCGTTTGGTTTCTCTGAGATCGTCATGATGAAGTACATCGCGTTCGGCATGATCGCAGCGCTG
ATTCTGGATGCGACCATCATCCGCATGCTGCTTGTCCCCCGCCGTGATGCACCTGCTTCGCGACGACAA
CTGGTGGGCACCCGGCTTCGT
RXS02587-3′-Region
TAAAAAGGCGTACACCGTCATGG
RXS02590-5′-Region
GCCCCAAAGGCTTAAAGTAATGGGCATGCCCACTGCTTCTTCGACCAAAAGCTACGCTGCGGTCTTACC
TCCACCTGGCCCCTCGTGGGCTGGTTCCCTC
RXS02590-coding Region
ATGGGCATCTCATTGTTGTCATCACTGTTGAAAATCCATGGTTTTCCAGTCGTCGCAGATTTCTTCTTC
GCGTTAGCTGTTGTGGTGGCAATTGTCATTATTGGCGGTTGGCTAATCTACCGCTCTCCTTCATTCAAA
ACTGAAGTCATGCCGGCATGGGCAATGCTGTCCATGGGTTTGATCGCATTGGGAACTGCAAGCCCCGTA
GTTTTGGGTGATGATCTGTGGGGATTTATGTTTGTGTGCTGGTCTATTGGCACAGCCGTGGGACTTGTT
GCCTATTCCTTATATATAACGGCCATTTTGCGATCTAAGGCGGGCAGACCAACTTTTGCGTGGGGTCTT
CCTCTTGTCACGCCGATGGTTGCTTCCACCTCGGCAGCACAACTCCATGAGCACTTTGAACTTCCGGCG
ATGCTGTGGGTTTGTTTCGGGCTCTTCCTTTTAACTTTGGCGTCTGCACCAGGAGTTTTTACCCGAGTG
TATTTCTACTATTTCGGCCCCAAGGCGCAGGGCATCCCACTGATGGCAACACCAACATCATGGATTCCT
TTGGGTATGGTGGGCCAATCCACTGCAGCAGCTCAGCTCATCGGTGCGTCCTTTGGATCCAAGACAGCA
ATCACAATGGGCATTATTTACGGCATCATCATGGGAATTTTTACGATTCCTCTGGGAGCCATCGCTCAC
TTTGTGTTCTACAGAGCTGTTTTCAAAGGGGCGACATACAGCCCCACATGGTGGGCCAGTACCTTCCCA
GTTGGCACTTTGAGTTTGGGTGGGCATTTTTTATCACAGAGCACCGGAGTGGAGTGGTTTAACTACTTC
AGCCTGTACTTGATTGCTTTAATGCTCTTTCATGTCATCGTGTCCACCATCGCCGGTACGATTGCAGTA
ATGAGAAGAATCGTCGGAAAGCTTAAATCTCAACTGGCC
RXS02590-3′-Region
TAAATTGCAGGGAGAGGTCTAAA
RXS02932-5′-Region
CACTACTGCGTTAAGGTATGAAAGTTCGCACACCAGCGATTTAATTGTGTGGCCACGACTAGCACGACC
ATTTCAGTTTTAACTTTCTTGGAGTTTTCTA
RXS02932-coding Region
GTGTCCAAAACAGAAGAAGGCCGTTCAGCGGGCATAATTATTTACGCGTTTCCAACTTTCATTCTGGTG
GGCGCGATCATTGCGTTTATCTTCCCGGAACCATTGATTCCGCTGACAAACTACATTAATATCTTCCTC
ACGATCATCATGTTCACCATGGGTTTGAGCTTGACGGTGCCCGATTTTCAGATGGTGGTTAAACGTCCA
CTGCCTATCTTGATCGGTGTAGTAGCGCAGTTTGTCATCATGCCATTCCTGGCGATCGTGGTTGCGAAA
ATGTTCAACCTCAACCCAGCACTGGCCGTTGGCCTTCTCATGCTGGGATCCGTTCCGGGTGGCACCTCC
TCCAATGTGATTGCGTTTCTCGCCCGAGGAGATGTCGCGCTATCGGTCACCATGACCTCTGTGTCCACC
ATTGTTTCCCCAATCATGACGCCTTTCCTGATGCTCATGCTGGCAGGTAGTGAAACCGCGGTCGATGGT
GGAGGCATGGCGTGGAGTTTGGTACAAACAGTGCTGGTGCCTGTGATCATCGGCCTAGTTCTGCGTGTC
TTCTTGAACAAGTGGATCGACAAGATTTTGCCGATCGTTCGTTATCTCTCCATCCTCGGTATCGGTGGC
GTGGTGTTCGGCGCAGTCGCAGCCAACGCGGAACGACTCGTGTCTGTCGGAGTCATCGTGTTCGTTGCA
GTTATCGTGCACAACGTACTTGGATACGTTGTGGGATACCTCACCGGCCGTGTATTCAAATTCCCAGAA
GGAGCAAACCGCACCATGGCGATTGAAATCGGAACCCAATCCGCAGGCCTGGCATCGGGAATGGCAGGA
CGATTCTTCACCCCAGAAGCAGCCCTTCCAGGTGCTGTCGCTGCCTTGGTCCACAACATCACCGGCGCA
GTTTATGTTGGGCTGGTACGAAACAGGCCTTTGACTAAGGCATCAAGGAAGAAGGAATCCGTCGCGGTT
TCCAGC
RXS02932-3′-Region
TAACTTATTTGCTGGCCGTTAGA
RXS03042-5′-Region
ATGACACCGGCGCGACGTATGGCATTACTGGCGTACCCCAATTTACGATGACATCTCTGCTCGCCTCGG
CGACGTCCTGGTTCCTTACGTTCTGATCGTT
RXS03042-coding Region
TTGGTTCTAGCGTTCCTCGTGCTGTTGCTCGTGTTCCGGTCCATTTGGGTCCCATTGATCGCGGCTCTG
GGCTTTGGCTTGTCAGTTCTGGCTACCTTTGGTGCTACGGTGGCGATCTTCCAAGAAGGTGCTTTCGGC
ATCATCGACGATCCTCAGGCACTGCTGTCCTTCTTGCCGATCATGCTCATCGGCGTGGTATTTGGTCTG
GCCATGGATTACCAGATCTTCCTCGTTACTCGTATGCGTGAGGGCTTGAGCAAGGGCAAGAGTGCGGGC
AACGCAACGTCGAATGGTTTCAAGCACGGTGCGCGCGTGGTCACTGCTGCGGGGCTGATCATGGTGTCT
GTGTTCGCGGCATTCATAGCGCAGGACATGGCGTTTATTAAGACCATGGGCTTTGCTCTGGGCGTTGCT
GTGTTCTTCGATGCCTTCGTTGTTCGCATGATGATTATCCCTGCAACAATGTTCCTGCTTGATGACAAG
GCTTGGTGGCTACCTAAGTGGTTGGATAAGATTCTTCCCAACGTTGATGTTGAAGGTGAGGGTCTTAGT
GAACTACATGAGGCTCGCACCGAGGAACTGAAGGAAAATGTAGGTGTCGGGGCT
RXS03042-3′-Region
TAGAGAAACAAAAAAGGCTGCTA
RXS03075-5′-Region
TGTGCAAAATTGCATTCAGGCTGAAAAATTCCTAAAGGGACTCCGTCCGAATAATTGGAAAGCCCAGAA
GAACAGTCAACTCCTAGATTAAAGGATAATC
RXS03075-coding Region
GTGGCGAAATTCCTGTATAAGTTAGGCTCCACGGCCTATCAAAAGAAATGGCCGTTTCTTGCGGTCTGG
CTCGTGATTCTCATAGGTATCACGACGCTGGCGGGGCTGTATGCCAAGCCAACGTCGAGTAGCTTCTCT
ATCCCTGGTCTTGATTCTGTCACGACCATGGAGAAGATGCAGGAGCGTTTCCCTGGTTCGGATGATGCA
ACATCGGCTCCCACTGGTTCTGTCGTCATTCAGGCACCGGAAGGCAAGACCCTCACTGATCCTGAGGTT
GGGGCTGAAGTAAACCAGATGCTTGATGAGGTTCGGGCGACTGGTGTGCTGAAGGATGCTGATTCCGTT
GTGGATCCTGTGTTGGCTGCGCAGGGTGTGGCTGCTCAGATGACCCCAGCCCTGGAGGCTGAGGGTGTA
CCTGCGGAGAAGATCGCCGCAGATATTGAGTCGATTAGTCCACTGAGTGCAGATGAGACTACCGGCATC
ATCTCGATGACTTTTGATGCAGATTCTGCCATGGATATATCCGCAGAGGATCGTGAGAAGGTCACCAAT
ATTCTTGATGAATACGATGACGGCGATCTGACTGTTGTCTACAACGGCAACGTGTTTGGCGCAGCTGCA
ACCAGCTTGGACATGACCTCTGAGCTCATCGGCCTGCTGGTGGCTGCGGTCGTTCTTATCGTGACCTTC
GGTTCGTTCATCGCTGCCGGTATGCCGCTGATCTCT
RXS03124-coding Region
ATGACTGCTACCCTGGCGTCGATGATTGGTCTGGCTGTGGGTATCGACTACGCGCTATTTATCGTGTCC
CGTTTCGGCAATGAGTTGATTTGTCAGACTGGCGCTAATGATCTGGAGCCAAAGGAATTGGCTGAGCGT
CTGCGCACCATGCCGTTGGCTGCTGGTGCGCATGCGATGGGAATGGCTGTGGGCACTGCGGGTTCTGCG
GTTGTATTCGCGGGTACCACGGTGCTGATCGCTCTGGTTGCTCTGTCGATCATTAATATTCCATTTCTA
ACCGTGATGGCCATTGCTGCCGCAATCACCGTTGCCATCGCAGTTCTGGTTGCTCTGTCCTTCCTCCCA
GCTGTGCTTGGCCTGGTTGGCACTGGCATCTTCGCAGCACGCGTGCCTGGACCTAAGGTTCCGGATCCT
GAGGACGAGAAGCCAACGATGGGTCTGAAGTGGGTCCGCCTTGTGCGCAAGATGCCGGTGGCTTACCTG
CTGGTTGGCGTCGTTTTGCTTGGTGCAATCGCAATTCCTGCGACCAATATGCGCCTGGCCATGCCGACT
GATGGCACCTCCACGCTGGGCACCGGGCCGCGCACGGGGTATGACATGACGGCAGATGCGTTCGGCCCG
GGCCGCAACGCGCCCATGATTGCGCTTATCGACGCAACCGACGTGCCTGAGGAAGAACGCCCATTGGTG
TTTGGACAGGCGGTGGAGCAATTCTTGAACACTGATGGTGTGAAGAATGCTCAGATCACTCAGACCACG
GAGAATTTCGATACCGCGCAGATCGTGTTAGCGCAGAATTTGATGCGATCGATGAGCGCACCTCTGAGA
CTCTCGCAACTCTTCGTGCAGATGCTGAGACCTTCGCTGATGACACCGGCGCGACGTATGGCATTACTG
GCGTCACCCCAATTTACGATGACATCTCTGCTCGCCTCGGCGACGTCCTGGTTCCTTACGTTC
RXS03124-3′-Region
TGATCGTTTTGGTTCTAGCGTTG
RXS03125-5′-Region
TGACACCGGCGCGACGTATGGCATTACTGGCGTCACCCCAATTTACGATGACATCTCTGCTCGCCTCGG
CGACGTCCTGGTTCCTTACGTTCTGATCGTT
RXS03125-coding Region
TTGGTTCTAGCGTTCCTCGTGCTGTTGCTCGTGTTCCGGTCCATTTGGGTCCCATTGATCGCGGCTCTG
GGCTTTGGCTTGTCAGTTCTGGCTACCTTTGGTGCTACCGTGGCGATCTTCCAAGAAGGTGCTTTCGGC
ATCATCGACGATCCTCAGCCACTGCTGTGCTTC
RXS03220-coding Region
ATGGGCTTAAGGGAAATTTTGTCCAGCAAGTGGCTTGTGCGCATCCTCCTGGTAGGTATCGGATTGGGT
GTCGCACAGCAGCTGACCGGCATCAACTCCATCATGTACTACGGCCAGGTTGTTCTCATTGAGGCTGGT
TTCTCCGAGAATGCAGCTCTGATCGCCAACGTGGCGCCAGGAGTGATCGCAGTTGTCGGTGCATTCATC
GCACTGTGGATGATGGATGGTATCAACCGCCGTACCACCGTCATTAGCGGTTATTCTCTCACCACCATT
AGCCACGTATTGATCGGTATCGCATGCGTAGCATTCCCAGTGGGCGATCCTCTTCGCCCCTACGTTATC
TTGACTCTGGTTGTGGTCTTGGTGGGATCGATGCAGAGCTTCCTCAACGTAGCTACCTGGGTTATGCTG
TCTGAGCTCTTCCCGCTGGCAATGCGCGGTTTGGCAATCGGTATCTCAGTGTTCTTCCTCTGGATCGCA
AACGCGTTCCTCGGATTGTTCTTGCCAACCATCATGGAAGCAGTAGGACTAACCGGAACCTTCTTCATG
TTCGCCGGAATCGGTGTGGTTGCCTTGATCTTCATCTAGACCCAGGTTCCTGAAACTCGTGGACGTACC
TTGGAGGAGATTGATGAGGATGTTACTTCCGGTGTCATTTTCAACAAGGACATCCGAAAAGGAAAGGTG
CAC
RXS03220-3′-Region
TAAAAACCCAGACACTGCATAGATAACACG
RXS03221-5′-Region
CAAAAGTATTCAAAAAAAGTTTGTTATGTACCATTGACGGGACATATCGTGTCTGCCACGATTAAAGAC
ATTGGTGATGTGAATCACTGCCTACTACATC
RXS03221-coding Region
GTGTTTCGTGACCCTGCACCTCCAAGTAAGGGCACGACAAACTTAGGAGACAAGATGGCTAGTACCTTC
ATTCAGGCCGACAGCCCTGAAAAAAGTAAGAAGCTGCCCCCACTCACAGAAGGTCCGTATAGAAAGCGG
CTATTCTACGTTGCACTAGTTGCGACGTTTGGTGGGCTGCTCTTCGGATATGACACCGGAGTAATCAAC
GGTGCACTCAACCCAATGACACGTGAGCTCGGACTAACCGCGTTCACCGAGGGTGTTGTAACTTCTTCC
CTGCTGTTTGGTGCAGCAGCTGGTGCGATGTTTTTCGGTCGCATTTCCGACAACTGGGGTCGCCGGAAA
ACAATCATCTCACTTGCAGTAGCTTTCTTTGTCGGCACCATGATCTGCGTGTTTGCTCCATCTTTTGCA
GTAATGGTTGTCGGACGTGTGCTTCTTGGACTCGCAGTTGGTGGCGCTTCCACTGTTGTCCCTGTCTAC
CTGGCTGAACTTGCTCCTTTTGAAATCCGTGGCTCACTGGCTGGCCGTAATGAGTTGATGATTGTTGTT
GGTCAGCTCGCAGCTTTTGTCATCAATGCGATTATTGGAAATGTTTTTGGACACCACGATGGTGTGTGG
CGCTACATGCTGGCAATTGCCGCAATCCCAGCAATTGCCCTCTTCTTTGGAATG

TABLE 4
ALIGNMENT RESULTS

| | length | | | | | | % homology | Date of |
|---|---|---|---|---|---|---|---|---|
| ID # | (NT) | Genbank Hit | Length | Accession | Name of Genbank Hit | Source of Genbank Hit | (GAP) | Deposit |
| rxa00001 | 1251 | GB_BA1:SRMSIK | 2384 | Y08921 | S. reticuli gene encoding Msik protein and orf1. | Streptomyces reticuli | 63,746 | 21-MAR-1997 |
| | | GB_BA1:SLU12007 | 1602 | U12007 | Streptomyces lividans 1326 ATP binding protein MsiK (msiK) gene, | Streptomyces lividans | 62,951 | 30-MAR-1996 |
| | | | | | complete cds. | | | |
| | | GB_BA1:MTCY16B7 | 43430 | Z81331 | Mycobacterium tuberculosis H37Rv complete genome; segment 123/162. | Mycobacterium | 41,425 | 17-Jun-98 |
| | | | | | | tuberculosis | | |
| rxa00002 | 807 | GB_BA1:MTV018 | 53450 | AL021899 | Mycobacterium tuberculosis H37Rv complete genome; segment 90/162. | Mycobacterium | 37,913 | 18-Jun-98 |
| | | | | | | tuberculosis | | |
| | | GB_EST22:AU020788 | 558 | AU020788 | AU020788 Mouse eight-cell stage embryo cDNA Mus musculus cDNA | Mus musculus | 38,757 | 19-OCT-1998 |
| | | | | | clone J0538B03 3′, mRNA sequence. | | | |
| | | GB_PR3:AC004160 | 143751 | AC004160 | Homo sapiens BAC clone GS164B05 from 7p21-p22, complete sequence. | Homo sapiens | 35,687 | 20-Feb-98 |
| rxa00089 | 1122 | GB_EST27:AI461009 | 570 | AI461009 | sa77g07.y1 Gm-c1004 Glycine max cDNA clone GENOME SYSTEMS | Glycine max | 37,833 | 01-DEC-1999 |
| | | | | | CLONE ID: Gm-c1004-5365 5′ similar to TR: O04014 O04014 RIBOSOMAL | | | |
| | | | | | PROTEIN S6 RPS6-1.;, mRNA sequence. | | | |
| | | GB_EST32:AI736780 | 474 | AI736780 | sb33d08.y1 Gm-c1012 Glycine max cDNA clone GENOME SYSTEMS | Glycine max | 37,367 | 06-DEC-1999 |
| | | | | | CLONE ID: Gm-c1012-232 5′ similar to TR: O04014 O04014 RIBOSOMAL | | | |
| | | | | | PROTEIN S6 RPS6-1.;, mRNA sequence. | | | |
| | | GB_EST30:AI637616 | 280 | AI637616 | tt10c03.x1 NCI_CGAP_GC6 Homo sapiens cDNA clone IMAGE: 2240356 | Homo sapiens | 37,455 | 27-Apr-99 |
| | | | | | 3′, mRNA sequence. | | | |
| rxa00090 | 1242 | GB_PL1:SCYOR023C | 1989 | Z74931 | S. cerevisiae chromosome XV reading frame ORF YOR023c. | Saccharomyces | 36,078 | 11-Aug-97 |
| | | | | | | cerevisiae | | |
| | | GB_GSS15:AQ659370 | 487 | AQ659370 | Sheared DNA-5C3.TR Sheared DNA Trypanosoma brucei genomic clone | Trypanosoma brucei | 44,920 | 23-Jun-99 |
| | | | | | Sheared DNA-5C3, genomic survey sequence. | | | |
| | | GB_PL1:MZEHSZEIN | 2123 | L29505 | Zea mays high sulfur zein gene, complete cds. | Zea mays | 37,245 | 24-MAR-1994 |
| rxa00099 | 1296 | GB_HTG1:HSDA14C6 | 155908 | AL049732 | Homo sapiens chromosome X clone RP6-14C6, *** SEQUENCING IN | Homo sapiens | 35,984 | 23-Nov-99 |
| | | | | | PROGRESS ***, in unordered pieces. | | | |
| | | GB_HTG1:HSDA14C6 | 155908 | AL049732 | Homo sapiens chromosome X clone RP6-14C6, *** SEQUENCING IN | Homo sapiens | 35,984 | 23-Nov-99 |
| | | | | | PROGRESS ***, in unordered pieces. | | | |
| | | GB_PL2:ATF22I13 | 93760 | AL035539 | Arabidopsis thaliana DNA chromosome 4, BAC clone F22I13 (ESSA | Arabidopsis thaliana | 35,161 | 27-Aug-99 |
| | | | | | project). | | | |
| rxa00123 | 1242 | GB_PL1:OS4CL | 5225 | X52623 | Rice 4-CL gene for 4-coumarate-CoA ligase (EC 6.2.1.12). | Oryza sativa | 39,330 | 7-Apr-93 |
| | | GB_BA1:AB020531 | 6445 | AB020531 | Escherichia coli plasmid pTZ3721 gene cluster containing the mphB gene | Escherichia coli | 36,923 | 20-Feb-99 |
| | | | | | for macrolide 2′-phosphotransferase II, complete cds. | | | |
| | | GB_PL1:OS4CL | 5225 | X52623 | Rice 4-CL gene for 4-coumarate-CoA ligase (EC 6.2.1.12). | Oryza sativa | 39,118 | 7-Apr-93 |
| rxa00160 | 696 | GB_EST11:AA270696 | 178 | AA270696 | va46g09.r1 Soares mouse 3NME12 5 Mus musculus cDNA clone | Mus musculus | 46,067 | 26-MAR-1997 |
| | | | | | IMAGE: 734464 5′ similar to gb: M17886 60S ACIDIC RIBOSOMAL | | | |
| | | | | | PROTEIN P1 (HUMAN); gb: U29402 Mus musculus acidic ribosomal | | | |
| | | | | | phosphoprotein P1 mRNA, complete (MOUSE);, mRNA sequence. | | | |
| | | GB_HTG3:AC010829 | 149101 | AC010829 | Homo sapiens clone 6_J_21, LOW-PASS SEQUENCE SAMPLING. | Homo sapiens | 36,880 | 23-Sep-99 |
| | | GB_HTG3:AC010829 | 149101 | AC010829 | Homo sapiens clone 6_J_21, LOW-PASS SEQUENCE SAMPLING. | Homo sapiens | 36,880 | 23-Sep-99 |
| rxa00193 | 594 | GB_PR3:AC005826 | 177585 | AC005826 | Homo sapiens clone UWGC: rg041a03 from 7p14-15, complete sequence. | Homo sapiens | 37,012 | 16-OCT-1998 |
| | | GB_HTG2:AC007076 | 95477 | AC007076 | Homo sapiens clone DJ0698F07, *** SEQUENCING IN PROGRESS ***, 1 | Homo sapiens | 37,012 | 5-Jun-99 |
| | | | | | unordered pieces. | | | |
| | | GB_HTG2:AC007076 | 95477 | AC007076 | Homo sapiens clone DJ0698F07, *** SEQUENCING IN PROGRESS ***, 1 | Homo sapiens | 37,012 | 5-Jun-99 |
| | | | | | unordered pieces. | | | |
| rxa00203 | 1035 | GB_GSS3:B88972 | 699 | B88972 | CIT-HSP-2173C11.TR CIT-HSP Homo sapiens genomic clone 2173C11, | Homo sapiens | 41,411 | 25-Jun-98 |
| | | | | | genomic survey sequence. | | | |
| | | GB_GSS11:AQ290299 | 642 | AQ290299 | nbxb0036N17r CUGI Rice BAC Library Oryza sativa genomic clone | Oryza sativa | 38,245 | 03-DEC-1998 |
| | | | | | nbxb0036N17r, genomic survey sequence. | | | |
| | | GB_EST20:AA843374 | 479 | AA843374 | aj16e10.s1 Soares_parathyroid_tumor_NbHPA Homo sapiens cDNA clone | Homo sapiens | 34,874 | 31-DEC-1998 |
| | | | | | IMAGE: 1390506 3′, mRNA sequence. | | | |
| rxa00204 | 1695 | GB_BA1:SC3F9 | 19830 | AL023862 | Streptomyces coelicolor cosmid 3F9. | Streptomyces coelicolor | 49,556 | 10-Feb-99 |
| | | GB_BA2:AF160811 | 10671 | AF160811 | Bacillus stearothermophilus L-arabinose transport, ATP binding protein | Bacillus | 51,004 | 28-Jul-99 |
| | | | | | (araG), L-arabinose membrane permease (araH), AraR (araR), L-ribulose 5- | stearothermophilus | | |
| | | | | | phosphate 4-epimerase (araD), L-ribulokinase (araB), L-arabinose | | | |
| | | | | | isomerase (araA), and IS5377 transposase genes, complete cds. | | | |
| | | GB_BA2:MPAE000056 | 16213 | AE000056 | Mycoplasma pneumoniae section 56 of 63 of the complete genome. | Mycoplasma | 34,895 | 18-Nov-96 |
| | | | | | | pneumoniae | | |
| rxa00270 | 1011 | GB_BA1:MLCB1770 | 37821 | Z70722 | Mycobacterium leprae cosmid B1770. | Mycobacterium leprae | 37,089 | 29-Aug-97 |
| | | GB_GSS4:AQ681972 | 452 | AQ681972 | HS_5503_B2_C02_T7A RPCI-11 Human Male BAC Library Homo sapiens | Homo sapiens | 40,099 | 28-Jun-99 |
| | | | | | genomic clone Plate = 1079 Col = 4 Row = F, genomic survey sequence. | | | |
| | | GB_VI:IVU47137 | 986 | U47137 | Inkoo virus Prototype KN3641 nucleocapsid protein and non-structural | Inkoo virus | 37,061 | 22-Aug-96 |
| | | | | | protein genes, complete cds. | | | |
| rxa00311 | 978 | GB_VI:VMVY16780 | 186986 | Y16780 | variola minor virus complete genome. | variola minor virus | 37,722 | 2-Sep-99 |
| | | GB_VI:VARCG | 186103 | L22579 | Variola major virus (strain Bangladesh-1975) complete genome. | Variola major virus | 38,558 | 12-Jan-95 |
| | | GB_VI:VVCGAA | 185578 | X69198 | Variola virus DNA complete genome. | Variola virus | 39,518 | 13-DEC-1996 |
| rxa00312 | 549 | GB_HTG4:AC011190 | 164409 | AC011190 | Homo sapiens clone hRPK.24_A_1, *** SEQUENCING IN PROGRESS | Homo sapiens | 34,741 | 19-OCT-1999 |
| | | | | | ***, 31 unordered pieces. | | | |
| | | GB_HTG4:AC011190 | 164409 | AC011190 | Homo sapiens clone hRPK.24_A_1, *** SEQUENCING IN PROGRESS | Homo sapiens | 34,741 | 19-OCT-1999 |
| | | | | | ***, 31 unordered pieces. | | | |
| | | GB_HTG4:AC011190 | 164409 | AC011190 | Homo sapiens clone hRPK.24_A_1, *** SEQUENCING IN PROGRESS | Homo sapiens | 34,807 | 19-OCT-1999 |
| | | | | | ***, 31 unordered pieces. | | | |
| rxa00345 | 1074 | GB_HTG2:AC007356 | 185382 | AC007356 | Drosophila melanogaster chromosome 2 clone BACR24H09 (D595) RPCI- | Drosophila | 36,364 | 2-Aug-99 |
| | | | | | 98 24.H.9 map 49A-49B strain y; cn bw sp, *** SEQUENCING IN | melanogaster | | |
| | | | | | PROGRESS ***, 13 unordered pieces. | | | |
| | | GB_HTG2:AC007356 | 185382 | AC0007356 | Drosophila melanogaster chromosome 2 clone BACR24H09 (D595) RPCI- | Drosophila | 36,364 | 2-Aug-99 |
| | | | | | 98 24.H.9 map 49A-49B strain y; cn bw sp, *** SEQUENCING IN | melanogaster | | |
| | | | | | PROGRESS ***, 13 unordered pieces. | | | |
| | | GB_HTG2:AC005814 | 183922 | AC005814 | Drosophila melanogaster chromosome 3 clone BACR48M07 (D471) RPCI- | Drosophila | 38,031 | 30-Jul-99 |
| | | | | | 98 48.M.7 map 64A6-64B6 strain y; cn bw sp, *** SEQUENCING IN | melanogaster | | |
| | | | | | PROGRESS ***, 11 unordered pieces. | | | |
| rxa00378 | 2733 | GB_BA2:ALW243431 | 26953 | AJ243431 | Acinetobacter Iwoffii wzc, wzb, wza, weeA, weeB, wceC, wzx, wzy, weeD, | Acinetobacter Iwoffii | 36,717 | 01-OCT-1999 |
| | | | | | weeE, weeF, weeG, weeH, weeI, weeJ, weeK, galU, ugd, pgi, galE, pgm | | | |
| | | | | | (partial) and mip (partial) genes (emulsan biosynthetic gene cluster), strain | | | |
| | | | | | RAG-1. | | | |
| | | GB_BA2:ALW243431 | 26953 | AJ243431 | Acinetobacter Iwoffii wzc, wzb, wza, weeA, weeB, wceC, wzx, wzy, weeD, | Acinetobacter Iwoffii | 36,394 | 01-OCT-1999 |
| | | | | | weeE, weeF, weeG, weeH, weeI, weeJ, weeK, galU, ugd, pgi, galE, pgm | | | |
| | | | | | (partial) and mip (partial) genes (emulsan biosynthetic gene cluster), strain | | | |
| | | | | | RAG-1. | | | |
| | | GB_RO:MMCOL3A1 | 43601 | X52046 | M. musculus COL3A1 gene for collagen alpha-I. | Mus musculus | 35,159 | 8-Nov-94 |
| rxa00412 | 1203 | GB_BA1:ECU70214 | 123171 | U70214 | Escherichia coli chromosome minutes 4-6. | Escherichia coli | 39,914 | 21-Sep-96 |
| | | GB_BA1:ECOTSF | 91430 | D83536 | Escherichia coli genomic DNA. (4.1-6.1 min). | Escherichia coli | 39,828 | 28-MAY-1999 |
| | | GB_HTG3:AC011366 | 177590 | AC011366 | Homo sapiens chromosome 5 clone CIT-HSPC_568L21, *** | Homo sapiens | 46,212 | 06-OCT-1999 |
| | | | | | SEQUENCING IN PROGRESS ***, 82 unordered pieces. | | | |
| rxa00413 | 1020 | GB_PR3:AC005209 | 184130 | AC005209 | Homo sapiens chromosome 17, clone hRPK.628_O_18, complete | Homo sapiens | 34,028 | 24-Jul-98 |
| | | | | | sequence. | | | |
| | | GB_PR3:HUMIL8R | 13089 | M99412 | Human interleukin-8 receptor (IL8RB) gene, complete cds. | Homo sapiens | 37,934 | 22-Apr-98 |
| | | GB_PR4:AC006974 | 90241 | AC006974 | Homo sapiens PAC clone DJ0958B11 from 7q33-q36, complete sequence. | Homo sapiens | 37,948 | 29-Jul-99 |
| rxa00431 | 912 | GB_BA1:MSGY126 | 37164 | AD000012 | Mycobacterium tuberculosis sequence from clone y126. | Mycobacterium | 66,776 | 10-DEC-1996 |
| | | | | | | tuberculosis | | |
| | | GB_BA1:MTY13D12 | 37085 | Z80343 | Mycobacterium tuberculosis H37Rv complete genome; segment 156/162. | Mycobacterium | 66,776 | 17-Jun-98 |
| | | | | | | tuberculosis | | |
| | | GB_BA1:MSGB971CS | 37566 | L78821 | Mycobacterium leprae cosmid B971 DNA sequence. | Mycobacterium leprae | 39,429 | 15-Jun-96 |
| rxa00444 | 960 | GB_PR4:AC007564 | 194058 | AC007564 | Homo sapiens 12q22 BAC RPCI11-513P18 (Roswell Park Cancer Institute | Homo sapiens | 35,220 | 3-Jul-99 |
| | | | | | Human BAC Library) complete sequence. | | | |
| | | GB_HTG4:AC007553 | 271496 | AC007553 | Homo sapiens chromosome 12q22-102.7-103.4 clone RPCI11-557K11, *** | Homo sapiens | 35,408 | 21-OCT-1999 |
| | | | | | SEQUENCING IN PROGRESS ***, 70 unordered pieces. | | | |
| | | GB_HTG4:AC007553 | 271496 | AC007553 | Homo sapiens chromosome 12q22-102.7-103.4 clone RPCI11-557K11, *** | Homo sapiens | 35,408 | 21-OCT-1999 |
| | | | | | SEQUENCING IN PROGRESS ***, 70 unordered pieces. | | | |
| rxa00445 | 1035 | GB_HTG3:AC011455_0 | 244238 | AC011455 | Homo sapiens chromosome 19 clone CIT-HSPC_360G5, *** | Homo sapiens | 35,455 | 19-Dec-99 |
| | | | | | SEQUENCING IN PROGRESS ***, 287 unordered pieces. | | | |
| | | GB_HTG3:AC011455_0 | 244238 | AC011455 | Homo sapiens chromosome 19 clone CIT-HSPC_360G5, *** | Homo sapiens | 35,455 | 19-Dec-99 |
| | | | | | SEQUENCING IN PROGRESS ***, 287 unordered pieces. | | | |
| | | GB_HTG3:AC011455_0 | 244238 | AC011455 | Homo sapiens chromosome 19 clone CIT-HSPC_360G5, *** | Homo sapiens | 41,511 | 19-Dec-99 |
| | | | | | SEQUENCING IN PROGRESS ***, 287 unordered pieces. | | | |
| rxa00466 | | | | | | | | |
| rxa00482 | 771 | GB_PR4:AF119709 | 43566 | AF119709 | Homo sapiens chromosome 8q24 BAC clone H103, complete sequence. | Homo sapiens | 36,724 | 28-Feb-99 |
| | | GB_RO:AC005960 | 158414 | AC005960 | Mus musculus chromosome 17 BAC citb20h22 from the MHC region, | Mus musculus | 39,836 | 01-DEC-1998 |
| | | | | | complete sequence. | | | |
| | | GB_RO:MUSMHH2M4X | 3994 | L14278 | Mouse MHC class I H2-M4 gene, exons 1-5. | Mus musculus | 32,713 | 11-Aug-93 |
| rxa00523 | 1149 | GB_BA2:AF176902 | 3032 | AF176902 | Corynebacterium diphtheriae IRP1B (irp1B), IRP1C (irp1C), and IRP1D | Corynebacterium | 58,781 | 5-Sep-99 |
| | | | | | (irp1D) genes, complete cds. | diphtheriae | | |
| | | GB_HTG3:AC002489 | 91638 | AC002489 | Mus musculus chromosome X clone 592 map X, *** SEQUENCING IN | Mus musculus | 37,819 | 3-Aug-99 |
| | | | | | PROGRESS ***, 8 unordered pieces. | | | |
| | | GB_HTG3:AC002489 | 91638 | AC002489 | Mus musculus chromosome X clone 592 map X, *** SEQUENCING IN | Mus musculus | 37,819 | 3-Aug-99 |
| | | | | | PROGRESS ***, 8 unordered pieces. | | | |
| rxa00525 | 1386 | GB_BA1:D90917 | 154619 | D90917 | Synechocystis sp. PCC6803 complete genome, 27/27, 3418852-3573470. | Synechocystis sp. | 46,966 | 7-Feb-99 |
| | | GB_PL1:AOF132610 | 477 | AJ132610 | Asparagus officinalis mRNA for intracellular pathogenesis-related protein, | Asparagus officinalis | 38,819 | 1-Feb-99 |
| | | | | | isoform 4. | | | |
| | | GB_PAT:A26571 | 737 | A26571 | A. officinalis AoPR1 gene. | Asparagus officinalis | 37,620 | 28-Sep-95 |
| rxa00596 | 576 | GB_PR3:AC004659 | 129577 | AC004659 | Homo sapiens chromosome 19, CIT-HSP-87m17 BAC clone, complete | Homo sapiens | 34,321 | 02-MAY-1998 |
| | | | | | sequence. | | | |
| | | GB_PR3:AC004659 | 129577 | AC004659 | Homo sapiens chromosome 19, CIT-HSP-87m17 BAC clone, complete | Homo sapiens | 35,739 | 02-MAY-1998 |
| | | | | | sequence. | | | |
| | | GB_PR1:HUMCBP2 | 2047 | D83174 | Human mRNA for collagen binding protein 2, complete cds. | Homo sapiens | 40,404 | 6-Feb-99 |
| rxa00634 | 1506 | GB_BA1:BRLBIOAD | 2272 | D14083 | Brevibacterium flavum genes for 7,8-diaminopelargonic acid | Corynebacterium | 39,111 | 3-Feb-99 |
| | | | | | aminotransferase and dethiobiotin synthetase, complete cds. | glutamicum | | |
| | | GB_PAT:E08643 | 285 | E08643 | Base sequence having the promoter function in Corynebacterium | Corynebacterium | 39,111 | 29-Sep-97 |
| | | | | | microorganisms. | glutamicum | | |
| | | GB_HTG2:AC006174 | 203407 | AC006174 | Homo sapiens chromosome 10 clone CIT987SK-1057L21 map 10q25, *** | Homo sapiens | 37,517 | 09-DEC-1998 |
| | | | | | SEQUENCING IN PROGRESS ***, 6 unordered pieces. | | | |
| rxa00665 | 601 | GB_BA1:SCI30A | 35033 | AL096811 | Streptomyces coelicolor cosmid I30A. | Streptomyces coelicolor | 38,095 | 22-Jul-99 |
| | | | | | | A3(2) | | |
| | | GB_PR3:AC002366 | 259202 | AC002366 | Human Xp22 BAC CT-285I15 (from CalTech/Research Genetics), PAC | Homo sapiens | 33,045 | 11-Jun-98 |
| | | | | | RPCI1-27C22 (from Roswell Park Cancer Center), and Cosmid U35B5 | | | |
| | | | | | (from Lawrence Livermore), complete sequence. | | | |
| | | GB_PR3:AC002366 | 259202 | AC002366 | Human Xp22 BAC CT-285I15 (from CalTech/Research Genetics), PAC | Homo sapiens | 35,214 | 11-Jun-98 |
| | | | | | RPCI1-27C22 (from Roswell Park Cancer Center), and Cosmid U35B5 | | | |
| | | | | | (from Lawrence Livermore), complete sequence. | | | |
| rxa00702 | 1830 | GB_BA1:PLNRTABC | 6449 | Z19598 | P. laminosum nrtA-PhI, nir-PhI, nrtB-PhI and nrtC-PhI genes. | Phormidium laminosum | 40,550 | 7-Feb-96 |
| | | GB_GSS10:AQ256518 | 704 | AQ256518 | nbxb0016M14r CUGI Rice BAC Library Oryza sativa genomic clone | Oryza sativa | 41,477 | 23-OCT-1998 |
| | | | | | nbxb0016M14r, genomic survey sequence. | | | |
| | | GB_BA1:AAC243194 | 1720 | AJ243194 | Alicyclobacillus acidocaldarius kdpA gene. | Alicyclobacillus | 39,740 | 21-Jun-99 |
| | | | | | | acidocaldarius | | |
| rxa00728 | 892 | GB_EST21:AA974252 | 426 | AA974252 | oq14a01.s1 NCI_CGAP_GC4 Homo sapiens cDNA clone IMAGE: 1586280 | Homo sapiens | 42,236 | 7-Jul-98 |
| | | | | | 3′ similar to SW:LIPA_ECOLI P25845 LIPOIC ACID SYNTHETASE; | | | |
| | | | | | contains MER22.t2 MER22 repetitive element;, mRNA sequence. | | | |
| | | GB_HTG2:AC004060 | 124000 | AC004060 | Homo sapiens chromosome 4, *** SEQUENCING IN PROGRESS ***, 10 | Homo sapiens | 38,106 | 21-Jul-98 |
| | | | | | unordered pieces. | | | |
| | | GB_HTG2:AC004060 | 124000 | AC004060 | Homo sapiens chromosome 4, *** SEQUENCING IN PROGRESS ***, 10 | Homo sapiens | 38,106 | 21-Jul-98 |
| | | | | | unordered pieces. | | | |
| rxa00732 | 1670 | GB_BA2:AE000241 | 10160 | AE000241 | Escherichia coli K-12 MG1655 section 131 of 400 of the complete genome. | Escherichia coli | 40,024 | 12-Nov-98 |
| | | GB_HTG3:AC010073 | 121859 | AC010073 | Homo sapiens chromosome 15 clone BAC 16E3 map 15q25, LOW-PASS | Homo sapiens | 39,001 | 11-Sep-99 |
| | | | | | SEQUENCE SAMPLING. | | | |
| | | GB_BA1:D90783 | 15399 | D90783 | E. coli genomic DNA, Kohara clone #272(32.4-32.7 min.). | Escherichia coli | 40,024 | 29-MAY-1997 |
| rxa00759 | 1047 | GB_BA1:MTV025 | 121125 | AL022121 | Mycobacterium tuberculosis H37Rv complete genome; segment 155/162. | Mycobacterium | 39,960 | 24-Jun-99 |
| | | | | | | tuberculosis | | |
| | | GB_PL1:BPNIR1 | 2472 | X60093 | B. pendula mRNA for nitrite reductase. | Betula pendula | 38,106 | 19-MAR-1992 |
| | | GB_BA1:MTV025 | 121125 | AL022121 | Mycobacterium tuberculosis H37Rv complete genome; segment 155/162. | Mycobacterium | 41,618 | 24-Jun-99 |
| | | | | | | tuberculosis | | |
| rxa00760 | 1155 | GB_BA2:AF092918 | 20758 | AF092918 | Pseudomonas alcaligenes outer membrane Xcp-secretion system gene | Pseudomonas | 40,450 | 06-DEC-1998 |
| | | | | | cluster. | alcaligenes | | |
| | | GB_BA1:SCI7 | 34893 | AL096743 | Streptomyces coelicolor cosmid I7. | Streptomyces coelicolor | 40,352 | 1-Jul-99 |
| | | GB_BA1:D90763 | 18199 | D90763 | E. coli genomic DNA, Kohara clone #252(28.1-28.4 min.). | Escherichia coli | 38,747 | 29-MAY-1997 |
| rxa00774 | 777 | GB_EST8:AA020814 | 419 | AA020814 | ze63h10.s1 Soares retina N2b4HR Homo sapiens cDNA clone | Homo sapiens | 37,500 | 30-Jan-97 |
| | | | | | IMAGE: 363715 3′ similar to PIR:A35715 A35715 fodrin alpha chain - | | | |
| | | | | | human;, mRNA sequence. | | | |
| | | GB_PL2:ATAC004521 | 104797 | AC004521 | Arabidopsis thaliana chromosome II BAC F4I1 genomic sequence, | Arabidopsis thaliana | 36,411 | 12-MAY-1998 |
| | | | | | complete sequence. | | | |
| | | GB_PL2:ATAC004521 | 104797 | AC004521 | Arabidopsis thaliana chromosome II BAC F4I1 genomic sequence, | Arabidopsis thaliana | 38,589 | 12-MAY-1998 |
| | | | | | complete sequence. | | | |
| rxa00775 | 894 | GB_BA1:MTV043 | 68848 | AL022004 | Mycobacterium tuberculosis H37Rv complete genome; segment 40/162. | Mycobacterium | 66,107 | 24-Jun-99 |
| | | | | | | tuberculosis | | |
| | | GB_BA2:AF045938 | 777 | AF045938 | Mycobacterium smegmatis putative ABC transporter nucleotide binding | Mycobacterium | 73,454 | 02-MAY-1998 |
| | | | | | subunit (mtp1) gene, complete cds. | smegmatis | | |
| | | GB_BA1:MLU15182 | 40123 | U15182 | Mycobacterium leprae cosmid B2266. | Mycobacterium leprae | 63,494 | 09-MAR-1995 |
| rxa00776 | 1044 | GB_PR3:HS453C12 | 147620 | AL021578 | Human DNA sequence from clone 453C12 on chromosome 20q12-13.12, | Homo sapiens | 35,833 | 23-Nov-99 |
| | | | | | complete sequence. | | | |
| | | GB_PR3:AC004877 | 128361 | AC004877 | Homo sapiens PAC clone DJ0751H13 from 7q35-qter, complete sequence. | Homo sapiens | 38,754 | 19-Sep-98 |
| | | GB_PR3:HS300I2 | 63796 | AL035660 | Human DNA sequence from clone 300I2 on chromosome 20q12-13.12, | Homo sapiens | 32,233 | 23-Nov-99 |
| | | | | | complete sequence. | | | |
| rxa00777 | 1188 | GB_BA1:ASAJ187 | 6213 | AJ000187 | Arthrobacter sp. catA gene. | Arthrobacter sp. | 49,694 | 5-Jul-99 |
| | | GB_IN1:CELT20D4 | 42052 | U80029 | Caenorhabditis elegans cosmid T20D4. | Caenorhabditis elegans | 36,457 | 04-DEC-1996 |
| | | GB_GSS4:AQ693388 | 531 | AQ693388 | HS_5458_A2_D10_T7A RPCI-11 Human Male BAC Library Homo sapiens | Homo sapiens | 38,123 | 6-Jul-99 |
| | | | | | genomic clone Plate = 1034 Col = 20 Row = G, genomic survey sequence. | | | |
| rxa00828 | 576 | GB_GSS1:CNS00ZMZ | 796 | AL097877 | Drosophila melanogaster genome survey sequence SP6 end of BAC | Drosophila | 39,286 | 26-Jul-99 |
| | | | | | BACN02F13 of DrosBAC library from Drosophila melanogaster (fruit fly), | melanogaster | | |
| | | | | | genomic survey sequence. | | | |
| | | GB_BA1:PSEBPH | 4169 | D16407 | Pseudomonas sp. bphE, bphG, bphF and ORF4 genes. | Pseudomonas sp. | 36,364 | 4-Feb-99 |
| | | GB_GSS9:AQ156606 | 668 | AQ156606 | nbxb0008K19r CUGI Rice BAC Library Oryza sativa genomic clone | Oryza sativa | 36,364 | 12-Sep-98 |
| | | | | | nbxb0008K19r, genomic survey sequence. | | | |
| rxa00832 | 1173 | GB_PR4:AC006504 | 210137 | AC006504 | Homo sapiens chromosome 19, BAC 326584 (CIT-B-459F4), complete | Homo sapiens | 40,545 | 4-Feb-99 |
| | | | | | sequence. | | | |
| | | GB_GSS12:AQ417775 | 642 | AQ417775 | RPCI-11-197B9.TVRPCI-11 Homo sapiens genomic clone RPCI-11- | Homo sapiens | 44,286 | 23-MAR-1999 |
| | | | | | 197B9, genomic survey sequence. | | | |
| | | GB_PR4:AC006504 | 210137 | AC006504 | Homo sapiens chromosome 19, BAC 326584 (CIT-B-459F4), complete | Homo sapiens | 35,886 | 4-Feb-99 |
| | | | | | sequence. | | | |
| rxa00934 | 1206 | GB_BA1:MLCL581 | 36225 | Z96801 | Mycobacterium leprae cosmid L581. | Mycobacterium leprae | 38,243 | 24-Jun-97 |
| | | GB_BA1:MTCY1A10 | 25949 | Z95387 | Mycobacterium tuberculosis H37Rv complete genome; segment 117/162. | Mycobacterium | 38,350 | 17-Jun-98 |
| | | | | | | tuberculosis | | |
| | | GB_PR3:HS434O14 | 135928 | AL022398 | Homo sapiens DNA sequence from PAC 434O14 on chromosome 1q32.3.-41. | Homo sapiens | 36,788 | 23-Nov-99 |
| | | | | | Contains the HSD11B1 gene for Hydroxysteroid (11-beta) | | | |
| | | | | | Dehydrogenase 1, the ADORA2BP adenosine A2b receptor LIKE | | | |
| | | | | | pseudogene, the IRF6 gene for Interferon Regulatory Factor 6 and two | | | |
| | | | | | novel genes. Contains ESTs and GSSs, complete sequence. | | | |
| rxa00939 | 1308 | GB_BA1:MTCY251 | 38380 | Z74410 | Mycobacterium tuberculosis H37Rv complete genome; segment 5/162. | Mycobacterium | 49,462 | 17-Jun-98 |
| | | | | | | tuberculosis | | |
| | | GB_PAT:I26656 | 3250 | I26656 | Sequence 1 from U.S. Pat. No. 5559011. | Unknown. | 49,462 | 07-OCT-1996 |
| | | GB_BA2:SCJ1 | 36925 | AL109962 | Streptomyces coelicolor cosmid J1. | Streptomyces coelicolor | 49,228 | 24-Sep-99 |
| | | | | | | A3(2) | | |
| rxa00942 | 327 | GB_IN1:CELT19D2 | 28406 | U42846 | Caenorhabditis elegans cosmid T19D2. | Caenorhabditis elegans | 43,910 | 19-DEC-1995 |
| | | GB_PR4:AC004905 | 134350 | AC004905 | Homo sapiens PAC clone DJ0845I21 from 7q11.21-q11.23, complete | Homo sapiens | 35,505 | 12-Jan-99 |
| | | | | | sequence. | | | |
| | | GB_IN1:CELF18C5 | 29095 | U29097 | Caenorhabditis elegans cosmid F18C5. | Caenorhabditis elegans | 37,107 | 15-Jun-95 |
| rxa00950 | 1029 | GB_BA1:SLTNRB | 2849 | X73633 | S. longisporoflavus TnrB gene. | Streptomyces | 52,255 | 9-Aug-94 |
| | | | | | | longisporoflavus | | |
| | | GB_BA1:MTCI364 | 29540 | Z93777 | Mycobacterium tuberculosis H37Rv complete genome; segment 52/162. | Mycobacterium | 38,872 | 17-Jun-98 |
| | | | | | | tuberculosis | | |
| | | GB_BA1:MSGY367 | 35336 | AD000008 | Mycobacterium tuberculosis sequence from clone y367. | Mycobacterium | 39,921 | 03-DEC-1996 |
| | | | | | | tuberculosis | | |
| rxa00960 | 1058 | GB_PL2:ATAC009325 | 105543 | AC009325 | Arabidopsis thaliana chromosome III BAC F4P13 genomic sequence, | Arabidopsis thaliana | 36,074 | 08-OCT-1999 |
| | | | | | complete sequence. | | | |
| | | GB_BA2:U59485 | 29078 | U59485 | Agrobacterium tumefaciens AtrC (atrC) gene, partial cds; AtrB (atrB), AtrA | Agrobacterium | 39,884 | 16-Jul-99 |
| | | | | | (atrA), AttA1 (attA1), AttA2 (attA2), AttB (attB), AttC (attC), AttD (attD), AttE | tumefaciens | | |
| | | | | | (attE), and AttF (attF) genes, complete cds; AttG (attG) gene, alternative | | | |
| | | | | | splice products, complete cds; AttH (attH), AttI (attI), AttJ (attJ), AttK (attK), | | | |
| | | | | | AttL (attL), AttM (attM), AttO (attO), AttP (attP), AttR (attR), AttS (attS), AttT | | | |
| | | | | | (attT), AttU (attU), attV (attV), AttW (attW), AttX (attX), AttY (attY), AttZ | | | |
| | | | | | (attZ), AtsA (atsA), AtsB (atsB), AtsC (atsC), and AtsD (atsD) genes, | | | |
| | | | | | complete cds; and AtsE (atsE) gene, partial cds. | | | |
| | | GB_PL2:ATAC009325 | 105543 | AC009325 | Arabidopsis thaliana chromosome III BAC F4P13 genomic sequence, | Arabidopsis thaliana | 36,162 | 08-OCT-1999 |
| | | | | | complete sequence. | | | |
| rxa00980 | 1917 | GB_BA1:MTCY10D7 | 39800 | Z79700 | Mycobacterium tuberculosis H37Rv complete genome; segment 44/162. | Mycobacterium | 48,176 | 17-Jun-98 |
| | | | | | | tuberculosis | | |
| | | GB_GSS10:AQ255373 | 639 | AQ255373 | mgxb0012D24r CUGI Rice Blast BAC Library Magnaporthe grisea genomic | Magnaporthe grisea | 38,624 | 23-OCT-1998 |
| | | | | | clone mgxb0012D24r, genomic survey sequence. | | | |
| | | GB_EST26:AU004809 | 728 | AU004809 | AU004809 Bombyx mori p50(Daizo) Bombyx mori cDNA clone ws20873, | Bombyx mori | 38,223 | 19-Jan-99 |
| | | | | | mRNA sequence. | | | |
| rxa01000 | | | | | | | | |
| rxa01002 | 927 | GB_BA2:AE001197 | 10039 | AE001197 | Treponema pallidum section 13 of 87 of the complete genome. | Treponema pallidum | 37,161 | 16-Jul-98 |
| | | GB_PL1:HVPGLYH | 3790 | Y10099 | H. vulgare mRNA for novel P-glycoprotein homologue. | Hordeum vulgare | 42,239 | 24-OCT-1997 |
| | | GB_IN1:AB003329 | 4328 | AB003329 | Leishmania amazonensis LaMDR1 multidrug resistance gene, complete | Leishmania | 40,176 | 24-MAR-1999 |
| | | | | | cds. | amazonensis | | |
| rxa01003 | 927 | GB_HTG2:HSJ168B21 | 67973 | AL118518 | Homo sapiens chromosome 6 clone RP1-168B21 map q26-27, *** | Homo sapiens | 35,159 | 03-DEC-1999 |
| | | | | | SEQUENCING IN PROGRESS ***, in unordered pieces. | | | |
| | | GB_HTG2:HSJ168B21 | 67973 | AL118518 | Homo sapiens chromosome 6 clone RP1-168B21 map q26-27, *** | Homo sapiens | 35,159 | 03-DEC-1999 |
| | | | | | SEQUENCING IN PROGRESS ***, in unordered pieces. | | | |
| | | GB_HTG2:HSJ168B21 | 67973 | AL118518 | Homo sapiens chromosome 6 clone RP1-168B21 map q26-27, *** | Homo sapiens | 39,956 | 03-DEC-1999 |
| | | | | | SEQUENCING IN PROGRESS ***, in unordered pieces. | | | |
| rxa01006 | 958 | GB_IN2:S74163 | 2630 | S74163 | Drosophila sp. T-related protein (Trg) mRNA, complete cds. | Drosophila sp. | 37,131 | 06-OCT-1999 |
| | | GB_PR4:AF130343 | 292721 | AF130343 | Homo sapiens chromosome 8 clone PAC 87.2 map 8q24.1, complete | Homo sapiens | 34,398 | 9-Jul-99 |
| | | | | | sequence. | | | |
| | | GB_HTG3:AC009415 | 186991 | AC009415 | Homo sapiens clone NH0576H09,*** SEQUENCING IN PROGRESS ***, | Homo sapiens | 36,325 | 21-Aug-99 |
| | | | | | 5 unordered pieces. | | | |
| rxa01012 | 1764 | GB_BA1:SYOATPBP | 2883 | D14438 | Synechococcus elongatus genes for ATP-binding protein and Mn- | Synechococcus | 50,346 | 3-Feb-99 |
| | | | | | stabilizing protein. | elongatus | | |
| | | GB_BA1:BSU20909 | 6404 | U20909 | Bacillus subtilis permease system App operon AppD (appD), AppF(appF), | Bacillus subtilis | 50,376 | 23-Feb-95 |
| | | | | | AppA (appA), AppB (appB), and AppC (appC) genes, complete cds. | | | |
| | | GB_BA2:ECOPOTABCD | 4385 | M64519 | E. coli transport protein (potA, potB, potC and potD) genes, complete cds. | Escherichia coli | 42,881 | 17-Jun-96 |
| rxa01013 | 818 | GB_IN2:AC005930 | 41284 | AC005930 | Leishmania major chromosome 3 clone L712 strain Friedlin, complete | Leishmania major | 40,444 | 13-Nov-99 |
| | | | | | sequence. | | | |
| | | GB_PR2:HS1110P6 | 40033 | AL049175 | Human DNA sequence from clone 1110P6 on chromosome Xq21.1-22.3. | Homo sapiens | 36,981 | 23-Nov-99 |
| | | | | | Contains a putative CpG island, complete sequence. | | | |
| | | GB_IN2:AC005930 | 41284 | AC005930 | Leishmania major chromosome 3 clone L712 strain Friedlin, complete | Leishmania major | 44,121 | 13-Nov-99 |
| | | | | | sequence. | | | |
| rxa01070 | 1509 | GB_BA2:U32795 | 10038 | U32795 | Haemophilus influenzae Rd section 110 of 163 of the complete genome. | Haemophilus influenzae | 44,668 | 29-MAY-1998 |
| | | | | | | Rd | | |
| | | GB_PR4:AC004985 | 159507 | AC004985 | Homo sapiens clone DJ1165K10, complete sequence. | Homo sapiens | 37,508 | 7-Aug-99 |
| | | GB_PR3:AC005244 | 127506 | AC005244 | Homo sapiens chromosome 17, clone hRPK.471_L_13, complete | Homo sapiens | 33,176 | 7-Aug-98 |
| | | | | | sequence. | | | |
| rxa01094 | 736 | GB_BA1:CORPYKI | 2795 | L27126 | Corynebacterium pyruvate kinase gene, complete cds. | Corynebacterium | 99,558 | 07-DEC-1994 |
| | | | | | | glutamicum | | |
| | | GB_BA1:SC4G6 | 36917 | AL096884 | Streptomyces coelicolor cosmid 4G6. | Streptomyces coelicolor | 37,569 | 23-Jul-99 |
| | | | | | | A3(2) | | |
| | | GB_HTG1:CNS01DS1 | 216986 | AL121612 | Homo sapiens chromosome 14 clone R-179A9, *** SEQUENCING IN | Homo sapiens | 38,577 | 15-OCT-1999 |
| | | | | | PROGRESS ***, in unordered pieces. | | | |
| rxa01141 | 948 | GB_HTG2:HSJ395C13 | 150336 | AL117344 | Homo sapiens chromosome 6 clone RP3-395C13 map q25.2-26, *** | Homo sapiens | 36,538 | 03-DEC-1999 |
| | | | | | SEQUENCING IN PROGRESS ***, in unordered pieces. | | | |
| | | GB_HTG2:HSJ395C13 | 150336 | AL117344 | Homo sapiens chromosome 6 clone RP3-395C13 map q25.2-26, *** | Homo sapiens | 36,538 | 03-DEC-1999 |
| | | | | | SEQUENCING IN PROGRESS ***, in unordered pieces. | | | |
| | | GB_HTG2:HSJ395C13 | 150336 | AL117344 | Homo sapiens chromosome 6 clone RP3-395C13 map q25.2-26, | Homo sapiens | 37,908 | 03-DEC-1999 |
| | | | | | ***SEQUENCING IN PROGRESS ***, in unordered pieces. | | | |
| rxa01142 | 621 | GB_BA1:CORAIA | 4705 | L09232 | Corynebacterium glutamicum acetohydroxy acid synthase (ilvB) and (ilvN) | Corynebacterium | 35,897 | 23-Feb-95 |
| | | | | | genes, and acetohydroxy acid isomeroreductase (ilvC) gene, complete cds. | glutamicum | | |
| | | GB_BA1:SCH35 | 45396 | AL078610 | Streptomyces coelicolor cosmid H35. | Streptomyces coelicolor | 52,295 | 4-Jun-99 |
| | | GB_BA2:AFACHRRA | 7390 | J05278 | Ralstonia eutropha ChrB (chrB), ChrA (chrA), ChrC (chrC), ChrD (chrD), | Ralstonia eutropha | 54,589 | 26-MAR-1999 |
| | | | | | YbiB (ybiB), pirin, and heat shock protein sigma-32 (RP32) genes, | | | |
| | | | | | complete cds. | | | |
| rxa01164 | 1758 | GB_GSS14:AQ555104 | 609 | AQ555104 | RPCI-11-415H1.TJ RPCI-11 Homo sapiens genomic clone RPCI-11- | Homo sapiens | 40,000 | 28-MAY-1999 |
| | | | | | 415H1, genomic survey sequence. | | | |
| | | GB_BA2:AE000309 | 13453 | AE000309 | Escherichia coli K-12 MG1655 section 199 of 400 of the complete genome. | Escherichia coli | 39,261 | 12-Nov-98 |
| | | GB_GSS14:AQ548213 | 668 | AQ548213 | RPCI-11-415H4.TV RPCI-11 Homo sapiens genomic clone RPCI-11- | Homo sapiens | 41,176 | 28-MAY-1999 |
| | | | | | 415H4, genomic survey sequence. | | | |
| rxa01168 | 933 | GB_BA1:MTV018 | 53450 | AL021899 | Mycobacterium tuberculosis H37Rv complete genome; segment 90/162. | Mycobacterium | 38,033 | 18-Jun-98 |
| | | | | | | tuberculosis | | |
| | | GB_PL2:ATAC003033 | 84254 | AC003033 | Arabidopsis thaliana chromosome II BAC T21L14 genomic sequence, | Arabidopsis thaliana | 37,486 | 19-DEC-1997 |
| | | | | | complete sequence. | | | |
| | | GB_PL2:ATAC003033 | 84254 | AC003033 | Arabidopsis thaliana chromosome II BAC T21L14 genomic sequence, | Arabidopsis thaliana | 38,142 | 19-DEC-1997 |
| | | | | | complete sequence. | | | |
| rxa01185 | 667 | GB_BA2:AF013987 | 3150 | AF013987 | Vibrio cholerae strain 0395 putative ABC transporter ATP-binding protein, | Vibrio cholerae | 44,128 | 21-MAY-1998 |
| | | | | | sigma54 (rpoN), putative sigma54 modulation protein and nitrogen | | | |
| | | | | | regulatory IIA protein (ptsN) genes, complete cds. | | | |
| | | GB_BA1:SASTPSMP | 1848 | Z30588 | S. aureus (RN4220) genes for potential ABC transporter and potential | Staphylococcus aureus | 43,402 | 25-MAY-1995 |
| | | | | | membrane spanning protein. | | | |
| | | GB_PR3:HS357I16 | 134506 | AL021921 | Homo sapiens DNA sequence from PAC 357I16 on chromosome 1p36.13. | Homo sapiens | 38,957 | 23-Nov-99 |
| | | | | | Contains GSSs, genomic marker D1S449 and a CA repeat polymorphism, | | | |
| | | | | | complete sequence. | | | |
| rxa01188 | 1227 | GB_PR3:HSN21F1 | 39212 | Z94162 | Human DNA sequence from cosmid N21F1 on chromosome 22 Contains | Homo sapiens | 37,277 | 23-Nov-99 |
| | | | | | exon trap and STS, complete sequence. | | | |
| | | GB_EST38:AW066174 | 455 | AW066174 | 687007C06.y1 687 —Early embryo from Delaware Zea mays cDNA, mRNA | Zea mays | 42,439 | 12-OCT-1999 |
| | | | | | sequence. | | | |
| | | GB_GSS4:AQ719542 | 493 | AQ719542 | HS_5529_B2_A02_SP6E RPCI-11 Human Male BAC Library Homo | Homo sapiens | 39,837 | 14-Jul-99 |
| | | | | | sapiens genomic clone Plate = 1105 Col = 4 Row = B, genomic survey | | | |
| | | | | | sequence. | | | |
| rxa01247 | 357 | GB_BA2:AF127374 | 63734 | AF127374 | Streptomyces lavendulae LinA homolog, cytochrome P450 hydroxylase | Streptomyces | 38,592 | 27-MAY-1999 |
| | | | | | ORF4, cytochrome P450 hydroxylase ORF3, MitT (mitT), MitS (mitS), MitR | lavendulae | | |
| | | | | | (mitR), MitQ (mitQ), MitP (mitP), MitO (mitO), MitN (mitN), MitM (mitM), | | | |
| | | | | | MitL (mitL), MitK (mitK), MitJ (mitJ), MitI (mitI), MitH (mitH), MitG (mitG), | | | |
| | | | | | MitF (mitF), MitE (mitE), MitD (mitD), MitC (mitC), MitB (mitB), MitA (mitA), | | | |
| | | | | | MmcA (mmcA), MmcB (mmcB), MmcC (mmcC), MmcD (mmcD), | | | |
| | | | | | MmcE (mmcE), MmcF (mmcF), MmcG (mmcG), MmcH (mmcH), MmcI | | | |
| | | | | | (mmcI), MmcJ (mmcJ), MmcK (mmcK), MmcL (mmcL), MmcM (mmcM), | | | |
| | | | | | MmcN (mmcN), MmcO (mmcO), Mrd (mrd), MmcP (mmcP), MmcQ | | | |
| | | | | | (mmcQ), MmcR (mmcR), MmcS (mmcS), MmcT (mmcT), MmcU (mmcU), | | | |
| | | | | | MmcV (mmcV), Mct (mct), MmcW (mmcW), MmcX (mmcX), and MmcY | | | |
| | | | | | (mmcY) genes, complete cds; and unknown genes. | | | |
| | | GB_BA2:AF127374 | 63734 | AF127374 | Streptomyces lavendulae LinA homolog, cytochrome P450 hydroxylase | Streptomyces | 45,915 | 27-MAY-1999 |
| | | | | | ORF4, cytochrome P450 hydroxylase ORF3, MitT (mitT), MitS (mitS), MitR | lavendulae | | |
| | | | | | (mitR), MitQ (mitQ), MitP (mitP), MitO (mitO), MitN (mitN), MitM (mitM), | | | |
| | | | | | MitL (mitL), MitK (mitK), MitJ (mitJ), MitI (mitI), MitH (mitH), MitG (mitG), | | | |
| | | | | | MitF (mitF), MitE (mitE), MitD (mitD), MitC (mitC), MitB (mitB), MitA (mitA), | | | |
| | | | | | MmcA (mmcA), MmcB (mmcB), MmcC (mmcC), MmcD (mmcD), | | | |
| | | | | | MmcE (mmcE), MmcF (mmcF), MmcG (mmcG), MmcH (mmcH), MmcI | | | |
| | | | | | (mmcI), MmcJ (mmcJ), MmcK (mmcK), MmcL (mmcL), MmcM (mmcM), | | | |
| | | | | | MmcN (mmcN), MmcO (mmcO), Mrd (mrd), MmcP (mmcP), MmcQ | | | |
| | | | | | (mmcQ), MmcR (mmcR), MmcS (mmcS), MmcT (mmcT), MmcU (mmcU), | | | |
| | | | | | MmcV (mmcV), Mct (mct), MmcW (mmcW), MmcX (mmcX), and MmcY | | | |
| | | | | | (mmcY) genes, complete cds; and unknown genes. | | | |
| | | GB_PR4:AC006039 | 176257 | AC006039 | Homo sapiens clone NH0319F03, complete sequence. | Homo sapiens | 30,899 | 05-MAY-1999 |
| rxa01285 | 749 | GB_BA1:SCI51 | 40745 | AL109848 | Streptomyces coelicolor cosmid I51. | Streptomyces coelicolor | 38,627 | 16-Aug-99 |
| | | | | | | A3(2) | | |
| | | GB_BA2:SCF34 | 38995 | AL109974 | Streptomyces coelicolor cosmid F34. | Streptomyces coelicolor | 38,586 | 24-Sep-99 |
| | | | | | | A3(2) | | |
| | | GB_BA2:MSU10425 | 4261 | U10425 | Mycobacterium smegmatis ferric exochelin uptake proteins FxuB (fxuB), | Mycobacterium | 61,230 | 07-DEC-1994 |
| | | | | | FxuA (fxuA) genes, complete cds, FxuC (fxuC) gene, partial cds, and ferric | smegmatis | | |
| | | | | | exochelin biosynthesis protein FxbA (fxbA) gene, complete cds. | | | |
| rxa01289 | 1167 | GB_BA1:SCI51 | 40745 | AL109848 | Streptomyces coelicolor cosmid I51. | Streptomyces coelicolor | 35,456 | 16-Aug-99 |
| | | | | | | A3(2) | | |
| | | GB_BA1:SCI51 | 40745 | AL109848 | Streptomyces coelicolor cosmid I51. | Streptomyces coelicolor | 37,576 | 16-Aug-99 |
| | | | | | | A3(2) | | |
| | | GB_EST31:AI704930 | 227 | AI704930 | UI-R-AB1-ys-c-07-0-UI.s1 UI-R-AB1 Rattus norvegicus cDNA clone UI-R- | Rattus norvegicus | 38,326 | 3-Jun-99 |
| | | | | | AB1-ys-c-07-0-UI 3′, mRNA sequence. | | | |
| rxa01290 | 1287 | GB_HTG2:AC006892 | 299081 | AC006892 | Caenorhabditis elegans clone Y69A2, *** SEQUENCING IN PROGRESS | Caenorhabditis elegans | 33,727 | 26-Feb-99 |
| | | | | | ***, 10 unordered pieces. | | | |
| | | GB_HTG2:AC006892 | 299081 | AC006892 | Caenorhabditis elegans clone Y69A2, *** SEQUENCING IN PROGRESS | Caenorhabditis elegans | 33,727 | 26-Feb-99 |
| | | | | | ***, 10 unordered pieces. | | | |
| | | GB_PR3:HS508I15 | 131353 | AL021707 | Human DNA sequence from clone 508I15 on chromosome 22q12-13 | Homo sapiens | 34,803 | 23-Nov-99 |
| | | | | | Contains gene for GTPBP1 (GTP binding protein 1), two novel genes | | | |
| | | | | | KIAA0063 and KIAA0668, an mRNA, ESTs, STSs, GSSs, a CA repeat | | | |
| | | | | | (D22S272) and CpG islands, complete sequence. | | | |
| rxa01297 | 921 | GB_BA1:MTCY16B7 | 43430 | Z81331 | Mycobacterium tuberculosis H37Rv complete genome; segment 123/162. | Mycobacterium | 38,133 | 17-Jun-98 |
| | | | | | | tuberculosis | | |
| | | GB_BA1:MSGY414A | 40121 | AD000007 | Mycobacterium tuberculosis sequence from clone y414a. | Mycobacterium | 61,716 | 03-DEC-1996 |
| | | | | | | tuberculosis | | |
| | | GB_HTG4:AC010181 | 185244 | AC010181 | Homo sapiens chromosome 3 seeders clone RPCI11-68L1, *** | Homo sapiens | 34,807 | 21-OCT-1999 |
| | | | | | SEQUENCING IN PROGRESS ***, 26 unordered pieces. | | | |
| rxa01298 | 1053 | GB_BA1:MTCY16B7 | 43430 | Z81331 | Mycobacterium tuberculosis H37Rv complete genome; segment 123/162. | Mycobacterium | 38,160 | 17-Jun-98 |
| | | | | | | tuberculosis | | |
| | | GB_BA1:MSGY414A | 40121 | AD000007 | Mycobacterium tuberculosis sequence from clone y414a. | Mycobacterium | 58,611 | 03-DEC-1996 |
| | | | | | | tuberculosis | | |
| | | GB_PL1:SCYJL013C | 2289 | Z49288 | S. cerevisiae chromosome X reading frame ORF YJL013c. | Saccharomyces | 36,180 | 11-Aug-97 |
| | | | | | | cerevisiae | | |
| rxa01303 | 1458 | GB_BA1:TTAJ5043 | 837 | AJ225043 | Thermus thermophilus partial narK gene. | Thermus thermophilus | 55,245 | 18-Jun-98 |
| | | GB_PL2:AC010675 | 84723 | AC010675 | Arabidopsis thaliana chromosome I BAC T17F3 genomic sequence, | Arabidopsis thaliana | 37,058 | 11-Nov-99 |
| | | | | | complete sequence. | | | |
| | | GB_GSS9:AQ170862 | 518 | AQ170862 | HS_3165_B2_F03_T7 CIT Approved Human Genomic Sperm Library D | Homo sapiens | 38,610 | 17-OCT-1998 |
| | | | | | Homo sapiens genomic clone Plate = 3165 Col = 6 Row = L, genomic survey | | | |
| | | | | | sequence. | | | |
| rxa01323 | 2388 | GB_BA1:MTCY10D7 | 39800 | Z79700 | Mycobacterium tuberculosis H37Rv complete genome; segment 44/162. | Mycobacterium | 53,376 | 17-Jun-98 |
| | | | | | | tuberculosis | | |
| | | GB_BA1:MTCY39 | 38500 | Z74025 | Mycobacterium tuberculosis H37Rv complete genome; segment 89/162. | Mycobacterium | 39,197 | 17-Jun-98 |
| | | | | | | tuberculosis | | |
| | | GB_BA1:MTCY251 | 38380 | Z74410 | Mycobacterium tuberculosis H37Rv complete genome; segment 5/162. | Mycobacterium | 52,698 | 17-Jun-98 |
| | | | | | | tuberculosis | | |
| rxa01338 | 1925 | GB_IN1:DROPROS | 6422 | M81389 | D. melanogaster Pros protein (prospero) mRNA, complete cds. | Drosophila | 37,229 | 26-Apr-93 |
| | | | | | | melanogaster | | |
| | | GB_EST9:AA060074 | 688 | AA060074 | mj73f07.r1 Soares mouse p3NMF19.5 Mus musculus cDNA clone | Mus musculus | 39,919 | 23-Sep-96 |
| | | | | | IMAGE: 481765 5′ similar to gb: X00246 Mouse mRNA with a Set 1 | | | |
| | | | | | repetitive element for a class I (MOUSE);, mRNA sequence. | | | |
| | | GB_EST16:AA560009 | 437 | AA560009 | vI16a01.r1 Stratagene mouse Tcell 937311 Mus musculus cDNA clone | Mus musculus | 37,071 | 18-Aug-97 |
| | | | | | IMAGE: 972360 5′, mRNA sequence. | | | |
| rxa01395 | 294 | GB_BA1:CGLYSEG | 2374 | X96471 | C. glutamicum lysE and lysG genes. | Corynebacterium | 38,462 | 24-Feb-97 |
| | | | | | | glutamicum | | |
| | | GB_IN1:CELF28B3 | 36262 | AF003136 | Caenorhabditis elegans cosmid F28B3. | Caenorhabditis elegans | 37,241 | 31-DEC-1997 |
| | | GB_GSS13:AQ486324 | 573 | AQ486324 | RPCI-11-264E18.TJ RPCI-11 Homo sapiens genomic clone RPCI-11- | Homo sapiens | 39,785 | 24-Apr-99 |
| | | | | | 264E18, genomic survey sequence. | | | |
| rxa01411 | 888 | GB_EST24:AU035428 | 756 | AU035428 | AU035428 Sugano mouse brain mncb Mus musculus cDNA clone MNCb- | Mus musculus | 37,112 | 08-OCT-1998 |
| | | | | | 0438, mRNA sequence. | | | |
| | | GB_GSS12:AQ399225 | 621 | AQ399225 | mgxb0019C11f CUGI Rice Blast BAC Library Magnaporthe grisea genomic | Magnaporthe grisea | 36,430 | 06-MAR-1999 |
| | | | | | clone mgxb0019C11f, genomic survey sequence. | | | |
| | | GB_EST18:T42211 | 337 | T42211 | 5474 Lambda-PRL2 Arabidopsis thaliana cDNA clone 111C20T7, mRNA | Arabidopsis thaliana | 42,433 | 7-Jan-98 |
| | | | | | sequence. | | | |
| rxa01454 | 367 | GB_GSS5:AQ818876 | 486 | AQ818876 | HS_5297_B1_E12_SP6E RPCI-11 Human Male BAC Library Homo | Homo sapiens | 36,585 | 26-Aug-99 |
| | | | | | sapiens genomic clone Plate = 873 Col = 23 Row = J, genomic survey | | | |
| | | | | | sequence. | | | |
| | | GB_EST19:AA778691 | 650 | AA778691 | af87h03.s1 Soares_testis_NHT Homo sapiens cDNA clone 1049045 3′ | Homo sapiens | 32,344 | 5-Feb-98 |
| | | | | | similar to contains L1.t2 L1 repetitive element;, mRNA sequence. | | | |
| | | GB_GSS15:AQ599724 | 543 | AQ599724 | HS_5354_B1_B01_T7A RPCI-11 Human Male BAC Library Homo sapiens | Homo sapiens | 39,773 | 10-Jun-99 |
| | | | | | genomic clone Plate = 930 Col = 1 Row = D, genomic survey sequence. | | | |
| rxa01455 | 585 | GB_PL2:AF002169 | 5217 | AF002169 | Neurospora crassa coxl translation protein CYA5 (cya5) gene, complete | Neurospora crassa | 39,161 | 24-MAR-1999 |
| | | | | | cds. | | | |
| | | GB_PL2:AF002169 | 5217 | AF002169 | Neurospora crassa coxl translation protein CYA5 (cya5) gene, complete | Neurospora crassa | 37,565 | 24-MAR-1999 |
| | | | | | cds. | | | |
| rxa01625 | 324 | GB_EST36:AV200593 | 300 | AV200593 | AV200593 Yuji Kohara unpublished cDNA Caenorhabditis elegans cDNA | Caenorhabditis elegans | 43,284 | 26-Jul-99 |
| | | | | | clone yk577f10 3′, mRNA sequence. | | | |
| | | GB_PL1:S48358 | 414 | S48358 | tRNA Trp [Saccharomyces cerevisiae, Genomic, 414 nt]. | Saccharomyces | 37,143 | 08-MAY-1993 |
| | | | | | | cerevisiae | | |
| | | GB_GSS8:AQ005856 | 390 | AQ005856 | CIT-HSP-2292G20.TR CIT-HSP Homo sapiens genomic clone 2292G20, | Homo sapiens | 39,205 | 27-Jun-98 |
| | | | | | genomic survey sequence. | | | |
| rxa01756 | 1431 | GB_HTG4:AC009886 | 163668 | AC009886 | Homo sapiens chromosome 15 clone 437_N_14 map 15, *** | Homo sapiens | 35,865 | 19-OCT-1999 |
| | | | | | SEQUENCING IN PROGRESS ***, 11 unordered pieces. | | | |
| | | GB_HTG4:AC009886 | 163668 | AC009886 | Homo sapiens chromosome 15 clone 437_N_14 map 15, *** | Homo sapiens | 35,865 | 19-OCT-1999 |
| | | | | | SEQUENCING IN PROGRESS ***, 11 unordered pieces. | | | |
| | | GB_EST6:N47950 | 424 | N47950 | yy84d12.s1 Soares_multiple_sclerosis_2NbHMSP Homo sapiens cDNA | Homo sapiens | 38,261 | 14-Feb-96 |
| | | | | | clone IMAGE: 280247 3′, mRNA sequence. | | | |
| rxa01808 | 1172 | GB_BA1:SEABCT | 1976 | X80735 | S. erythraea (NCIMB 8594) ertX gene for putative ABC tranporter. | Saccharopolyspora | 63,607 | 07-DEC-1995 |
| | | | | | | erythraea | | |
| | | GB_BA1:MTV047 | 10866 | AL022002 | Mycobacterium tuberculosis H37Rv complete genome; segment 75/162. | Mycobacterium | 40,563 | 17-Jun-98 |
| | | | | | | tuberculosis | | |
| | | GB_BA1:ECOUW93 | 338534 | U14003 | Escherichia coli K-12 chromosomal region from 92.8 to 00.1 minutes. | Escherichia coli | 35,112 | 17-Apr-96 |
| rxa01822 | 605 | GB_HTG3:AC008480 | 106822 | AC008480 | Homo sapiens chromosome 5 clone CIT-HSPC_397O13, *** | Homo sapiens | 35,940 | 3-Aug-99 |
| | | | | | SEQUENCING IN PROGRESS ***, 36 unordered pieces. | | | |
| | | GB_HTG3:AC008480 | 106822 | AC008480 | Homo sapiens chromosome 5 clone CIT-HSPC_397O13, *** | Homo sapiens | 35,940 | 3-Aug-99 |
| | | | | | SEQUENCING IN PROGRESS ***, 36 unordered pieces. | | | |
| | | GB_PL2:CNS01B8L | 660 | AL113917 | Botrytis cinerea strain T4 cDNA library under conditions of nitrogen | Botryotinia fuckeliana | 43,322 | 2-Sep-99 |
| | | | | | deprivation. | | | |
| rxa01900 | 1422 | GB_BA2:AF056309 | 4346 | AF056309 | Streptomyces argillaceus membrane protein and mithramycin regulator | Streptomyces | 39,199 | 27-Jan-99 |
| | | | | | MtmR (mtmR) genes, complete cds. | argillaceus | | |
| | | GB_EST25:AU045582 | 273 | AU045582 | AU045582 Mouse sixteen-cell-embryo cDNA Mus musculus cDNA clone | Mus musculus | 61,172 | 09-DEC-1998 |
| | | | | | J0937H06 3′, mRNA sequence. | | | |
| | | GB_EST15:AA458642 | 217 | AA458642 | aa16b10.s1 Soares_NhHMPu_S1 Homo sapiens cDNA clone | Homo sapiens | 43,056 | 9-Jun-97 |
| | | | | | IMAGE: 813403 3′ similar to TR: G496330 G496330 IKBL MRNA.;, mRNA | | | |
| | | | | | sequence. | | | |
| rxa01939 | 1854 | GB_BA1:MTV025 | 121125 | AL022121 | Mycobacterium tuberculosis H37Rv complete genome; segment 155/162. | Mycobacterium | 38,145 | 24-Jun-99 |
| | | | | | | tuberculosis | | |
| | | GB_BA1:SC2A11 | 22789 | AL031184 | Streptomyces coelicolor cosmid 2A11. | Streptomyces coelicolor | 45,783 | 5-Aug-98 |
| | | GB_BA2:AE000431 | 11575 | AE000431 | Escherichia coli K-12 MG1655 section 321 of 400 of the complete genome. | Escherichia coli | 38,384 | 12-Nov-98 |
| rxa01972 | 717 | GB_HTG2:AC007650 | 166670 | AC007650 | Drosophila melanogaster chromosome 3 clone BACR30G22 (D688) RPCI- | Drosophila | 37,712 | 2-Aug-99 |
| | | | | | 98 30.G.22 map 87F-87F strain y; cn bw sp, *** SEQUENCING IN | melanogaster | | |
| | | | | | PROGRESS ***, 101 unordered pieces. | | | |
| | | GB_HTG2:AC007650 | 166670 | AC007650 | Drosophila melanogaster chromosome 3 clone BACR30G22 (D688) RPCI- | Drosophila | 37,712 | 2-Aug-99 |
| | | | | | 98 30.G.22 map 87F-87F strain y; cn bw sp, *** SEQUENCING IN | melanogaster | | |
| | | | | | PROGRESS ***, 101 unordered pieces. | | | |
| | | GB_HTG2:AC008204 | 138364 | AC008204 | Drosophila melanogaster chromosome 3 clone BACR04E17 (D762) RPCI- | Drosophila | 36,827 | 2-Aug-99 |
| | | | | | 98 04.E.17 map 95E-95F strain y; cn bw sp, *** SEQUENCING IN | melanogaster | | |
| | | | | | PROGRESS***, 96 unordered pieces. | | | |
| rxa01995 | 1461 | GB_HTG7:AC008065 | 172383 | AC008065 | Homo sapiens clone RP11-284E18, *** SEQUENCING IN PROGRESS ***, | Homo sapiens | 37,213 | 09-DEC-1999 |
| | | | | | 4 unordered pieces. | | | |
| | | GB_GSS5:AQ805794 | 426 | AQ805794 | HS_3192_A2_C04_MR CIT Approved Human Genomic Sperm Library D | Homo sapiens | 41,148 | 9-Aug-99 |
| | | | | | Homo sapiens genomic clone Plate = 3192 Col = 8 Row = E, genomic survey | | | |
| | | | | | sequence. | | | |
| | | GB_GSS10:AQ173736 | 436 | AQ173736 | HS_3194_A1_C04_MR CIT Approved Human Genomic Sperm Library D | Homo sapiens | 40,421 | 17-OCT-1998 |
| | | | | | Homo sapiens genomic clone Plate = 3194 Col = 7 Row = E, genomic survey | | | |
| | | | | | sequence. | | | |
| rxa02034 | 1089 | GB_PR4:AC002531 | 197900 | AC002531 | Homo sapiens chromosome Y, clone 486_O_8, complete sequence. | Homo sapiens | 36,934 | 13-OCT-1999 |
| | | GB_PR2:HSB7L1C4 | 106710 | AL078476 | Homo sapiens chromosome 21 BAC B7L1C4, complete sequence. | Homo sapiens | 34,454 | 9-Nov-99 |
| | | GB_IN2:CELF26D11 | 36161 | AF068716 | Caenorhabditis elegans cosmid F26D11. | Caenorhabditis elegans | 36,524 | 29-MAY-1998 |
| rxa02035 | | | | | | | | |
| rxa02062 | 1293 | GB_BA1:MTCI364 | 29540 | Z93777 | Mycobacterium tuberculosis H37Rv complete genome; segment 52/162. | Mycobacterium | 38,606 | 17-Jun-98 |
| | | | | | | tuberculosis | | |
| | | GB_EST34:AV153141 | 305 | AV153141 | AV153141 Mus musculus hippocampus C57BL/6J adult Mus musculus | Mus musculus | 37,705 | 7-Jul-99 |
| | | | | | cDNA clone 2900053B17, mRNA sequence. | | | |
| | | GB_BA1:PHU88400 | 3855 | U88400 | Prochlorothrix hollandica hoxUYH operon, hydrogenase diaphorase subunit | Prochlorothrix | 38,712 | 05-MAY-1997 |
| | | | | | (hoxU) gene, partial cds, and bidirectional hydrogenase small subunit | hollandica | | |
| | | | | | (hoxY), unknown protein, and bidirectional hydrogenase large subunit | | | |
| | | | | | (hoxH) genes, complete cds. | | | |
| rxa02068 | 1230 | GB_GSS13:AQ488513 | 673 | AQ488513 | RPCI-11-243J24.TV RPCI-11 Homo sapiens genomic clone RPCI-11- | Homo sapiens | 36,567 | 24-Apr-99 |
| | | | | | 243J24, genomic survey sequence. | | | |
| | | GB_GSS13:AQ488513 | 673 | AQ488513 | RPCI-11-243J24.TV RPCI-11 Homo sapiens genomic clone RPCI-11- | Homo sapiens | 36,567 | 24-Apr-99 |
| | | | | | 243J24, genomic survey sequence. | | | |
| rxa02079 | 738 | GB_PR4:AC006531 | 167525 | AC006531 | Homo sapiens chromosome 16 clone 113K5, complete sequence. | Homo sapiens | 37,870 | 7-Feb-99 |
| | | GB_BA1:DLARGD | 1471 | L42615 | Deleya cupida 16S ribosomal RNA (16S rRNA) gene. | Halomonas cupida | 40,476 | 3-Jan-96 |
| | | GB_BA1:AF009342 | 1482 | AF009342 | Haemophilus ducreyi ribosomal protein L11 gene, partial cds, and | Haemophilus ducreyi | 34,813 | 22-Jul-97 |
| | | | | | ribosomal protein L1 gene, complete cds. | | | |
| rxa02096 | 1815 | GB_BA1:MTV033 | 21620 | AL021928 | Mycobacterium tuberculosis H37Rv complete genome; segment 11/162. | Mycobacterium | 48,302 | 17-Jun-98 |
| | | | | | | tuberculosis | | |
| | | GB_BA2:MSU10425 | 4261 | U10425 | Mycobacterium smegmatis ferric exochelin uptake proteins FxuB (fxuB), | Mycobacterium | 41,282 | 07-DEC-1994 |
| | | | | | FxuA (fxuA) genes, complete cds, FxuC (fxuC) gene, partial cds, and ferric | smegmatis | | |
| | | | | | exochelin biosynthesis protein FxbA (fxbA) gene, complete cds. | | | |
| | | GB_EST30:AV018477 | 249 | AV018477 | AV018477 Mus musculus 18-day embryo C57BL/6J Mus musculus cDNA | Mus musculus | 42,169 | 28-Aug-99 |
| | | | | | clone 1190005G23, mRNA sequence. | | | |
| rxa02119 | 1764 | GB_BA1:SCARD1GN | 2321 | X84374 | S. capreolus ard1 gene. | Streptomyces capreolus | 49,857 | 23-Aug-95 |
| | | GB_PL2:SPBC29A3 | 42770 | AL022299 | S. pombe chromosome II cosmid c29A3. | Schizosaccharomyces | 37,269 | 02-DEC-1999 |
| | | | | | | pombe | | |
| | | GB_HTG1:CEY47H10 | 296589 | Z95311 | Caenorhabditis elegans chromosome I clone Y47H10, *** SEQUENCING | Caenorhabditis elegans | 34,160 | 7-Sep-99 |
| | | | | | IN PROGRESS ***, in unordered pieces. | | | |
| rxa02200 | 1233 | GB_PR3:HSA494O16 | 50502 | AL117328 | Human DNA sequence from clone 494O16 on chromosome 22, complete | Homo sapiens | 38,648 | 23-Nov-99 |
| | | | | | sequence. | | | |
| | | GB_HTG2:AC008161 | 158440 | AC008161 | Mus musculus clone 182_H_5 *** SEQUENCING IN PROGRESS ***, 29 | Mus musculus | 35,938 | 28-Jul-99 |
| | | | | | unordered pieces. | | | |
| | | GB_HTG2:AC008161 | 158440 | AC008161 | Mus musculus clone 182_H_5, *** SEQUENCING IN PROGRESS ***, 29 | Mus musculus | 35,938 | 28-Jul-99 |
| | | | | | unordered pieces. | | | |
| rxa02222 | | | | | | | | |
| rxa02312 | 1482 | GB_BA1:ECOUW93 | 338534 | U14003 | Escherichia coli K-12 chromosomal region from 92.8 to 00.1 minutes. | Escherichia coli | 60,729 | 17-Apr-96 |
| | | GB_BA2:AE000492 | 10181 | AE000492 | Escherichia coli K-12 MG1655 section 382 of 400 of the complete genome. | Escherichia coli | 60,729 | 12-Nov-98 |
| | | GB_BA1:BSUB0004 | 213190 | Z99107 | Bacillus subtilis complete genome (section 4 of 21): from 600701 to | Bacillus subtilis | 35,670 | 26-Nov-97 |
| | | | | | 813890. | | | |
| rxa02313 | 1344 | GB_EST30:AV013722 | 344 | AV013722 | AV013722 Mus musculus 18-day embryo C57BL/6J Mus musculus cDNA | Mus musculus | 39,941 | 25-Aug-99 |
| | | | | | clone 1110049L02, mRNA sequence. | | | |
| | | GB_EST29:AI596306 | 356 | AI596306 | ve20b05.y1 Soares mouse NbMH Mus musculus cDNA clone | Mus musculus | 40,395 | 21-Apr-99 |
| | | | | | IMAGE: 818673 5′, mRNA sequence. | | | |
| | | GB_EST29:AI595357 | 335 | AI595357 | ve20b05.x1 Soares mouse NbMH Mus musculus cDNA clone | Mus musculus | 35,821 | 21-Apr-99 |
| | | | | | IMAGE: 818673 3′, mRNA sequence. | | | |
| rxa02348 | | | | | | | | |
| rxa02353 | 491 | GB_BA2:AF175299 | 8140 | AF175299 | Sinorhizobium meliloti ThuR (thuR), ThuE (thuE), ThuF (thuF), ThuG | Sinorhizobium meliloti | 51,674 | 30-Aug-99 |
| | | | | | (thuG), ThuK (thuK), ThuA (thuA), and ThuB (thuB) genes, complete cds. | | | |
| | | GB_HTG3:AC008675 | 206439 | AC008675 | Homo sapiens chromosome 5 clone CIT978SKB_45I8 *** SEQUENCING | Homo sapiens | 38,351 | 3-Aug-99 |
| | | | | | IN PROGRESS ***, 43 unordered pieces. | | | |
| | | GB_HTG3:AC008672 | 131573 | AC008672 | Homo sapiens chromosome 5 clone CIT978SKB_3B12, *** SEQUENCING | Homo sapiens | 40,289 | 3-Aug-99 |
| | | | | | IN PROGRESS ***, 71 unordered pieces. | | | |
| rxa02354 | 957 | GB_PR3:HS357K22 | 145638 | AL022720 | Human DNA sequence from clone 357K22 on chromosome Xq27.1-27.3 | Homo sapiens | 34,926 | 23-Nov-99 |
| | | | | | Contains EST, STS, GSS, complete sequence. | | | |
| | | GB_IN1:D87738 | 1849 | D87738 | Branchiostoma belcheri mRNA for cytoplasmic actin BbCA1, complete cds. | Branchiostoma belcheri | 39,788 | 2-Apr-99 |
| | | GB_IN1:PIOACTA | 4172 | M26501 | Starfish (P. ochraceus) cytoplasmic actin gene, complete cds. | Pisaster ochraceus | 40,178 | 26-Apr-93 |
| rxa02394 | 1434 | GB_HTG5:AC007521 | 173897 | AC007521 | Drosophila melanogaster chromosome X clone BACR49A04 (D698) RPCI- | Drosophila | 34,954 | 17-Nov-99 |
| | | | | | 98 49.A.4 map 10A2-10B2 strain y; cn bw sp, *** SEQUENCING IN | melanogaster | | |
| | | | | | PROGRESS ***, 56 unordered pieces. | | | |
| | | GB_HTG3:AC010357 | 302201 | AC010357 | Homo sapiens chromosome 5 clone CITB-H1_2030B19, *** | Homo sapiens | 38,411 | 15-Sep-99 |
| | | | | | SEQUENCING IN PROGRESS ***, 12 unordered pieces. | | | |
| | | GB_HTG3:AC010357 | 302201 | AC010357 | Homo sapiens chromosome 5 clone CITB-H1_2030B19, *** | Homo sapiens | 38,411 | 15-Sep-99 |
| | | | | | SEQUENCING IN PROGRESS ***, 12 unordered pieces. | | | |
| rxa02438 | 882 | GB_BA1:SC7B7 | 13800 | AL009199 | Streptomyces coelicolor cosmid 7B7. | Streptomyces coelicolor | 53,592 | 02-DEC-1997 |
| | | GB_BA1:SC3F9 | 19830 | AL023862 | Streptomyces coelicolor cosmid 3F9. | Streptomyces coelicolor | 47,166 | 10-Feb-99 |
| | | GB_BA1:SCF43A | 35437 | AL096837 | Streptomyces coelicolor cosmid F43A. | Streptomyces coelicolor | 44,987 | 13-Jul-99 |
| | | | | | | A3(2) | | |
| rxa02439 | 1146 | GB_PAT:E16763 | 2517 | E16763 | gDNA encoding aspartate transferase (AAT). | Corynebacterium | 39,600 | 28-Jul-99 |
| | | | | | | glutamicum | | |
| | | GB_HTG1:HSDJ753D5 | 184025 | AL049693 | Homo sapiens chromosome 6 clone RP4-753D5, *** SEQUENCING IN | Homo sapiens | 36,894 | 23-Nov-99 |
| | | | | | PROGRESS ***, in unordered pieces. | | | |
| | | GB_HTG1:HSDJ753D5 | 184025 | AL049693 | Homo sapiens chromosome 6 clone RP4-753D5, *** SEQUENCING IN | Homo sapiens | 36,894 | 23-Nov-99 |
| | | | | | PROGRESS ***, in unordered pieces. | | | |
| rxa02441 | 780 | GB_EST7:W61724 | 312 | W61724 | md66b02.r1 Soares mouse embryo NbME13.5 14.5 Mus musculus cDNA | Mus musculus | 42,308 | 7-Jun-96 |
| | | | | | clone IMAGE: 373323 5′ similar to gb: L23769 Mouse microfibril-associated | | | |
| | | | | | glycoprotein (MOUSE);, mRNA sequence. | | | |
| | | GB_PR4:AC008115 | 158431 | AC008115 | Homo sapiens 12p12-27.2-31.7 BAC RPCI11-180M15 (Roswell Park | Homo sapiens | 37,286 | 09-OCT-1999 |
| | | | | | Cancer Institute Human BAC Library) complete sequence. | | | |
| | | GB_EST29:AI622043 | 586 | AI622043 | 486032B05.x2 486 - leaf primordia cDNA library from Hake lab Zea mays | Zea mays | 36,364 | 22-Apr-99 |
| | | | | | cDNA, mRNA sequence. | | | |
| rxa02442 | 972 | GB_PR4:AC007870 | 134757 | AC007870 | Genomic sequence for Homo sapiens clone 4P6, complete sequence. | Homo sapiens | 37,110 | 12-Aug-99 |
| | | GB_PR4:AC007870 | 134757 | AC007870 | Genomic sequence for Homo sapiens clone 4P6, complete sequence. | Homo sapiens | 34,941 | 12-Aug-99 |
| | | GB_PAT:AR053765 | 4624 | AR053765 | Sequence 5 from U.S. Pat. No. 5834263. | Unknown. | 38,988 | 29-Sep-99 |
| rxa02447 | 1118 | GB_EST36:AI881490 | 551 | AI881490 | 606069G06.y1 606 - Ear tissue cDNA library from Schmidt lab Zea mays | Zea mays | 49,534 | 21-Jul-99 |
| | | | | | cDNA, mRNA sequence. | | | |
| | | GB_PL1:CKRNAHUP3 | 1605 | X75440 | C. kessleri HUP3 mRNA. | Chlorella kessleri | 42,935 | 28-Jun-95 |
| | | GB_PL1:CKHUP1 | 2481 | Y07520 | Chlorella kessleri HUP1 mRNA for H(+)/hexose cotransporter. | Chlorella kessleri | 43,379 | 12-Sep-93 |
| rxa02451 | 1647 | GB_BA1:BRLBIOAD | 2272 | D14083 | Brevibacterium flavum genes for 7,8-diaminopelargonic acid | Corynebacterium | 40,496 | 3-Feb-99 |
| | | | | | aminotransferase and dethiobiotin synthetase, complete cds. | glutamicum | | |
| | | GB_PAT:E08643 | 285 | E08643 | Base sequence having the promoter function in Corynebacterium | Corynebacterium | 37,193 | 29-Sep-97 |
| | | | | | microorganisms. | glutamicum | | |
| | | GB_PL2:EFI245745 | 3194 | AJ245745 | Endomyces fibuliger ura3 gene for orotidine-5′-phosphate decarboxylase. | Saccharomycopsis | 38,889 | 24-Aug-99 |
| | | | | | | fibuligera | | |
| rxa02491 | 1377 | GB_BA1:MTCY20G9 | 37218 | Z77162 | Mycobacterium tuberculosis H37Rv complete genome; segment 25/162. | Mycobacterium | 55,745 | 17-Jun-98 |
| | | | | | | tuberculosis | | |
| | | GB_BA1:CGLEUA | 3492 | X70959 | C. glutamicum gene leuA for isopropylmalate synthase. | Corynebacterium | 38,253 | 10-Feb-99 |
| | | | | | | glutamicum | | |
| | | GB_PR3:AC004764 | 68048 | AC004764 | Homo sapiens chromosome 5, P1 clone 255g5 (LBNL H61), complete | Homo sapiens | 34,821 | 29-MAY-1998 |
| | | | | | sequence. | | | |
| rxa02507 | 1524 | GB_PR4:AC000134 | 203300 | AC000134 | Homo sapiens Chromosome 11q13 BAC Clone 137c7, complete sequence. | Homo sapiens | 37,087 | 06-MAY-1999 |
| | | GB_PR2:HS227L5 | 85304 | AL031585 | Human DNA sequence from clone 227L5 on chromosome Xp11.22-11.3. | Homo sapiens | 38,718 | 23-Nov-99 |
| | | | | | Contains a Keratin, Type 1 Cytoskeletal 18 (KRT18, CYK18, K18, CK18) | | | |
| | | | | | pseudogene and an STS, complete sequence. | | | |
| | | GB_PR4:AC000134 | 203300 | AC000134 | Homo sapiens Chromosome 11q13 BAC Clone 137c7, complete sequence. | Homo sapiens | 35,955 | 06-MAY-1999 |
| rxa02515 | 879 | GB_BA1:SCC22 | 22115 | AL096839 | Streptomyces coelicolor cosmid C22. | Streptomyces coelicolor | 36,219 | 12-Jul-99 |
| | | GB_BA1:MTV007 | 32806 | AL021184 | Mycobacterium tuberculosis H37Rv complete genome; segment 64/162. | Mycobacterium | 63,026 | 17-Jun-98 |
| | | | | | | tuberculosis | | |
| | | GB_BA1:MLCL536 | 36224 | Z99125 | Mycobacterium leprae cosmid L536. | Mycobacterium leprae | 36,468 | 04-DEC-1998 |
| rxa02562 | 843 | GB_HTG7:AC011197 | 167967 | AC011197 | Homo sapiens clone RP11-322C8, *** SEQUENCING IN PROGRESS ***, | Homo sapiens | 36,675 | 08-DEC-1999 |
| | | | | | 19 unordered pieces. | | | |
| | | GB_PAT:AR008238 | 6553 | AR008238 | Sequence 1 from U.S. Pat. No. 5753442. | Unknown. | 39,251 | 04-DEC-1998 |
| | | GB_GSS4:AQ712494 | 469 | AQ712494 | HS_2137_A1_A12_T7C CIT Approved Human Genomic Sperm Library D | Homo sapiens | 35,664 | 13-Jul-99 |
| | | | | | Homo sapiens genomic clone Plate = 2137 Col = 23 Row = A, genomic survey | | | |
| | | | | | sequence. | | | |
| rxa02595 | 1287 | GB_BA1:MSGB983CS | 36788 | L78828 | Mycobacterium leprae cosmid B983 DNA sequence. | Mycobacterium leprae | 39,905 | 15-Jun-96 |
| | | GB_BA1:MLCB1883 | 43505 | AL022486 | Mycobacterium leprae cosmid B1883. | Mycobacterium leprae | 52,909 | 27-Aug-99 |
| | | GB_GSS1:CNS0056G | 994 | AL057090 | Drosophila melanogaster genome survey sequence T7 end of BAC # | Drosophila | 31,315 | 3-Jun-99 |
| | | | | | BACR11M23 of RPCI-98 library from Drosophila melanogaster (fruit fly), | melanogaster | | |
| | | | | | genomic survey sequence. | | | |
| rxa02597 | | | | | | | | |
| rxa02605 | 618 | GB_BA1:MXENO201 | 390 | X92571 | M. xenopi gene for 32 kDa protein (partial). | Mycobacterium xenopi | 55,738 | 15-Jan-98 |
| | | GB_BA1:MXENO201 | 390 | X92571 | M. xenopi gene for 32 kDa protein (partial). | Mycobacterium xenopi | 59,233 | 15-Jan-98 |
| rxa02614 | 852 | GB_BA1:SCH35 | 45396 | AL078610 | Streptomyces coelicolor cosmid H35. | Streptomyces coelicolor | 50,976 | 4-Jun-99 |
| | | GB_BA2:AF126201 | 12402 | AF126201 | Pseudomonas putida strain S-313 sulfate ester desulfurization gene locus, | Pseudomonas putida | 46,763 | 12-OCT-1999 |
| | | | | | complete sequence. | | | |
| | | GB_BA1:SC8B7 | 14634 | AL031225 | Streptomyces coelicolor cosmid 8B7. | Streptomyces coelicolor | 38,026 | 7-Aug-98 |
| rxa02616 | 834 | GB_BA1:SCD78 | 36224 | AL034355 | Streptomyces coelicolor cosmid D78. | Streptomyces coelicolor | 43,705 | 26-Nov-98 |
| | | GB_EST28:AI509984 | 534 | AI509984 | mj18e06.y1 Soares mouse embryo NbME13.5 14.5 Mus musculus cDNA | Mus musculus | 38,653 | 12-MAR-1999 |
| | | | | | clone IMAGE: 476482 5′, mRNA sequence. | | | |
| | | GB_EST8:AA050633 | 522 | AA050633 | mj18e06.r1 Soares mouse embryo NbME13.5 14.5 Mus musculus cDNA | Mus musculus | 41,602 | 9-Sep-96 |
| | | | | | clone IMAGE: 476482 5′, mRNA sequence. | | | |
| rxa02627 | 866 | GB_GSS6:AQ826046 | 427 | AQ826046 | HS_5311_B2_B01_SP6E RPCI-11 Human Male BAC Library Homo | Homo sapiens | 38,095 | 27-Aug-99 |
| | | | | | sapiens genomic clone Plate = 887 Col = 2 Row = D, genomic survey | | | |
| | | | | | sequence. | | | |
| | | GB_PR2:HS329F2 | 24753 | AL031710 | Human DNA sequence from clone LA16-329F2 on chromosome 16, | Homo sapiens | 38,580 | 22-Nov-99 |
| | | | | | complete sequence. | | | |
| | | GB_GSS10:AQ255771 | 621 | AQ255771 | nbxb0014E22r CUGI Rice BAC Library Oryza sativa genomic clone | Oryza sativa | 34,622 | 23-OCT-1998 |
| | | | | | nbxb0014E22r, genomic survey sequence. | | | |
| rxa02628 | 528 | GB_BA1:RCAHIMA | 5403 | M84030 | Rhodobacter capsulatus integration host factor (himA) gene, complete cds. | Rhodobacter capsulatus | 37,452 | 26-Apr-93 |
| | | GB_GSS13:AQ476201 | 312 | AQ476201 | CITBI-E1-2592P3.TF CITBI-E1 Homo sapiens genomic clone 2592P3, | Homo sapiens | 43,182 | 23-Apr-99 |
| | | | | | genomic survey sequence. | | | |
| | | GB_EST38:AW054154 | 648 | AW054154 | 614079C04.x1 614 - root cDNA library from Walbot Lab Zea mays cDNA, | Zea mays | 37,657 | 21-Sep-99 |
| | | | | | mRNA sequence. | | | |
| rxa02650 | 702 | GB_EST19:AA803900 | 441 | AA803900 | GM14564.5prime GM Drosophila melanogaster ovary pOT2 Drosophila | Drosophila | 40,394 | 25-Nov-98 |
| | | | | | melanogaster cDNA clone GM14564 5prime, mRNA sequence. | melanogaster | | |
| | | GB_EST19:AA803900 | 441 | AA803900 | GM14564.5prime GM Drosophila melanogaster ovary pOT2 Drosophila | Drosophila | 37,757 | 25-Nov-98 |
| | | | | | melanogaster cDNA clone GM14564 5prime, mRNA sequence. | melanogaster | | |
| rxa02660 | 762 | GB_PR3:HS308O1 | 166715 | Z93403 | Human genomic DNA sequence from clone 308O1 on chromosome Xp11.3-11.4. | Homo sapiens | 33,912 | 23-Nov-99 |
| | | | | | Contains EST, CA repeat, STS, GSS, CpG island. | | | |
| | | GB_PR3:AC003669 | 159446 | AC003669 | Homo sapiens Xp22 BAC GS-594A7 (Genome Systems Human BAC | Homo sapiens | 35,734 | 24-MAR-1998 |
| | | | | | library) contains Bmx gene, complete sequence. | | | |
| | | GB_HTG3:AC010923 | 152021 | AC010923 | Drosophila melanogaster chromosome X clone BACR19K15 (D897) RPCI- | Drosophila | 28,070 | 08-OCT-1999 |
| | | | | | 98 19.K.15 map 15B-15E strain y; cn bw sp, *** SEQUENCING IN | melanogaster | | |
| | | | | | PROGRESS ***, 175 unordered pieces. | | | |
| rxa02661 | 342 | GB_HTG2:AC007802 | 118569 | AC007802 | Drosophila melanogaster chromosome 2 clone BACR07I11 (D648) RPCI- | Drosophila | 43,373 | 2-Aug-99 |
| | | | | | 98 07.I.11 map 58A1-58A2 strain y; cn bw sp, *** SEQUENCING IN | melanogaster | | |
| | | | | | PROGRESS ***, 70 unordered pieces. | | | |
| | | GB_HTG2:AC007802 | 118569 | AC007802 | Drosophila melanogaster chromosome 2 clone BACR07I11 (D648) RPCI- | Drosophila | 43,373 | 2-Aug-99 |
| | | | | | 98 07.I.11 map 58A1-58A2 strain y; cn bw sp, *** SEQUENCING IN | melanogaster | | |
| | | | | | PROGRESS ***, 70 unordered pieces. | | | |
| | | GB_EST2:R04660 | 288 | R04660 | pk27b04.r1 Kuwabara Mixed stage C. briggsae Caenorhabditis briggsae | Caenorhabditis | 47,009 | 31-MAR-1995 |
| | | | | | cDNA, mRNA sequence. | briggsae | | |
| rxa02663 | 1518 | GB_BA1:SC9F2 | 11908 | AL035559 | Streptomyces coelicolor cosmid 9F2. | Streptomyces coelicolor | 45,964 | 25-Feb-99 |
| | | GB_BA1:MTCY50 | 36030 | Z77137 | Mycobacterium tuberculosis H37Rv complete genome; segment 55/162. | Mycobacterium | 38,998 | 17-Jun-98 |
| | | | | | | tuberculosis | | |
| | | GB_BA1:D90721 | 16578 | D90721 | Escherichia coli genomic DNA. (18.6-19.0 min). | Escherichia coli | 44,325 | 7-Feb-99 |
| rxa02664 | 783 | GB_BA2:U32798 | 10423 | U32798 | Haemophilus influenzae Rd section 113 of 163 of the complete genome. | Haemophilus influenzae | 39,868 | 29-MAY-1998 |
| | | | | | | Rd | | |
| | | GB_BA1:HIU17295 | 9424 | U17295 | Haemophilus influenzae dppB, dppC, dppD, dppF, isn, artP, artl/J, artQ, | Haemophilus influenzae | 49,298 | 4-Apr-96 |
| | | | | | and artM genes, complete cds, and opa gene, partial cds. | | | |
| | | GB_BA2:U32792 | 11306 | U32792 | Haemophilus influenzae Rd section 107 of 163 of the complete genome. | Haemophilus influenzae | 39,764 | 29-MAY-1998 |
| | | | | | | Rd | | |
| rxa02684 | 987 | GB_PR2:CNS00006 | 181433 | AL049775 | Human chromosome 14 DNA sequence *** IN PROGRESS *** BAC R- | Homo sapiens | 36,961 | 17-Jun-99 |
| | | | | | 497E19 of RPCI-11 library from chromosome 14 of Homo sapiens | | | |
| | | | | | (Human), complete sequence. | | | |
| | | GB_HTG3:AC009857 | 148241 | AC009857 | Homo sapiens clone 2_F_6, *** SEQUENCING IN PROGRESS ***, 9 | Homo sapiens | 35,380 | 3-Sep-99 |
| | | | | | unordered pieces. | | | |
| | | GB_HTG3:AC009857 | 148241 | AC009857 | Homo sapiens clone 2_F_6, *** SEQUENCING IN PROGRESS ***, 9 | Homo sapiens | 35,380 | 3-Sep-99 |
| | | | | | unordered pieces. | | | |
| rxa02728 | 936 | GB_BA1:YEHEMSTUV | 3901 | X77867 | Y. enterocolitica hemS, hemT, hemU and hemV genes. | Yersinia enterocolitica | 48,253 | 11-OCT-1996 |
| | | GB_BA1:ECOUW76 | 225419 | U00039 | E. coli chromosomal region from 76.0 to 81.5 minutes. | Escherichia coli | 39,177 | 7-Nov-96 |
| | | GB_HTG3:AC008616 | 112626 | AC008616 | Homo sapiens chromosome 19 clone CIT978SKB_144D21, *** | Homo sapiens | 41,741 | 3-Sep-99 |
| | | | | | SEQUENCING IN PROGRESS ***, 49 unordered pieces. | | | |
| rxa02750 | 939 | GB_GSS15:AQ663436 | 430 | AQ663436 | HS_2160_B2_F10_T7C CIT Approved Human Genomic Sperm Library D | Homo sapiens | 42,020 | 23-Jun-99 |
| | | | | | Homo sapiens genomic clone Plate = 2160 Co = 20 Row = L, genomic survey | | | |
| | | | | | sequence. | | | |
| | | GB_GSS15:AQ663436 | 430 | AQ663436 | HS_2160_B2_F10_T7C CIT Approved Human Genomic Sperm Library D | Homo sapiens | 39,161 | 23-Jun-99 |
| | | | | | Homo sapiens genomic clone Plate = 2160 Col = 20 Row = L, genomic survey | | | |
| | | | | | sequence. | | | |
| rxa02795 | 1560 | GB_HTG5:AC011134 | 192982 | AC011134 | Homo sapiens clone 1_A_23, *** SEQUENCING IN PROGRESS ***, 22 | Homo sapiens | 35,630 | 5-Nov-99 |
| | | | | | unordered pieces. | | | |
| | | GB_HTG5:AC011134 | 192982 | AC011134 | Homo sapiens clone 1_A_23, *** SEQUENCING IN PROGRESS ***, 22 | Homo sapiens | 34,643 | 5-Nov-99 |
| | | | | | unordered pieces. | | | |
| | | GB_BA1:MTCY50 | 36030 | Z77137 | Mycobacterium tuberculosis H37Rv complete genome; segment 55/162. | Mycobacterium | 39,934 | 17-Jun-98 |
| | | | | | | tuberculosis | | |
| rxa02808 | 281 | GB_PR4:AC004897 | 90731 | AC004897 | Homo sapiens PAC clone DJ0811N16 from 7q34-q36, complete sequence. | Homo sapiens | 42,804 | 19-Aug-99 |
| | | GB_RO:AC002121 | 84056 | AC002121 | Genomic sequence from Mouse 11, complete sequence. | Mus musculus | 39,130 | 10-Jul-97 |
| | | GB_PR4:AC005078 | 73231 | AC005078 | Homo sapiens BAC clone RG252K19 from 7p15.2-p21, complete | Homo sapiens | 37,175 | 18-MAR-1999 |
| | | | | | sequence. | | | |
| rxs03220 | 725 | GB_PL1:CKHUP2 | 2353 | X66855 | C. kessleri HUP2 mRNA. | Chlorella kessleri | 45,328 | 17-Feb-97 |
| | | GB_EST38:AW048153 | 383 | AW048153 | UI-M-BH1-alq-h-05-0-UI.s1 NIH_BMAP_M_S2 Mus musculus cDNA clone | Mus musculus | 41,758 | 18-Sep-99 |
| | | | | | UI-M-BH1-alq-h-05-0-UI 3′, mRNA sequence. | | | |
| | | GB_PL1:CKHUP2 | 2353 | X66855 | C. kessleri HUP2 mRNA. | Chlorella kessleri | 38,106 | 17-Feb-97 |
| rxs03221 | 776 | GB_BA1:BSUB0010 | 233780 | Z99113 | Bacillus subtilis complete genome (section 10 of 21): from 1781201 | Bacillus subtilis | 52,282 | 28-Nov-97 |
| | | | | | to 2014980. | | | |
| | | GB_BA1:BSU66480 | 26114 | U66480 | Bacillus subtilis SpoVK (spoVK), YnbA (ynbA), YnbB (ynbB), GlnR | Bacillus subtilis | 52,282 | 22-Jan-97 |
| | | | | | (glnR), glutamine synthetase (glnA), YnaA (ynaA), YnaB (ynaB), YnaC | | | |
| | | | | | (ynaC), YnaD (ynaD), YnaE (ynaE), YnaF (ynaF), YnaG (ynaG), YnaH | | | |
| | | | | | (ynaH), YnaI (ynaI), YnaJ (ynaJ), xylan beta-1,4-xylosidase (xynB), | | | |
| | | | | | xylose repressor (xyIR), xylose isomerase (xyIA), xylulose kinase | | | |
| | | | | | (xyIB), YncB (yncB), YncC (yncC), YncD (yncD) and YncE (yncE) | | | |
| | | | | | genes, complete cds. | | | |
| | | GB_BA1:BSUB0010 | 233780 | Z99113 | Bacillus subtilis complete genome (section 10 of 21): from 1781201 | Bacillus subtilis | 36,983 | 26-Nov-97 |
| | | | | | to 2014980. | | | |

TABLE 2
GENES IDENTIFIED FROM GENBANK

| GenBank ™ | | | |
|---|---|---|---|
| Accession | | | |
| No. | Gene Name | Gene Function | Reference |
| A09073 | ppg | Phosphoenol pyruvate carboxylase | Bachmann, B. et al. “DNA fragment coding for phosphoenolpyruvat |
| | | | corboxylase, recombinant DNA carrying said fragment, strains carrying the |
| | | | recombinant DNA and method for producing L-aminino acids using said |
| | | | strains,” Patent: EP 0358940-A 3 Mar. 21, 1990 |
| A45579, | | Threonine dehydratase | Moeckel, B. et al. “Production of L-isoleucine by means of recombinant |
| A4581, | | | micro-organisms with deregulated threonine dehydratase,” Patent: WO |
| A45583, | | | 9519442-A 5 Jul. 20, 1995 |
| A45585 | | | |
| A45587 | | | |
| AB003132 | murC; ftsQ; | | Kobayashi, M. et al. “Cloning, sequencing, and characterization of the ftsZ |
| | ftsZ | | gene from coryneform bacteria,” Biochem. Biophys. Res. Commun., |
| | | | 236(2): 383-388 (1997) |
| AB015023 | murC; ftsQ | | Wachi, M. et al. “A murC gene from Coryneform bacteria,” Appl. Microbiol. |
| | | | Biotechnol., 51(2): 223-228 (1999) |
| AB018530 | dtsR | | Kimura, E. et al. “Molecular cloning of a novel gene, dtsR, which rescues the |
| | | | detergent sensitivity of a mutant derived from Brevibacterium |
| | | | lactofermentum,” Biosci. Biotechnol. Biochem., 60(10): 1565-1570 (1996) |
| AB018531 | dtsR1; dtsR2 | | |
| AB020624 | murI | D-glutamate racemase | |
| AB023377 | tkt | transketolase | |
| AB024708 | gltB; gltD | Glutamine 2-oxoglutarate | |
| | | aminotransferase | |
| | | large and small subunits | |
| AB025424 | acn | aconitase | |
| AB027714 | rep | Replication protein | |
| AB027715 | rep; aad | Replication protein; aminoglycoside | |
| | | adenyltransferase | |
| AF005242 | argC | N-acetylglutamate-5-semialdehyde | |
| | | dehydrogenase | |
| AF005635 | glnA | Glutamine synthetase | |
| AF030405 | hisF | cyclase | |
| AF030520 | argG | Argininosuccinate synthetase | |
| AF031518 | argF | Ornithine carbamolytransferase | |
| AF036932 | aroD | 3-dehydroquinate dehydratase | |
| AF038548 | pyc | Pyruvate carboxylase | |
| AF038651 | dciAE; apt; | Dipeptide-binding protein; adenine | Wehmeier, L. et al. “The role of the Corynebacterium glutamicum rel gene in |
| | rel | phosphoribosyltransferase; GTP | (p)ppGpp metabolism,” Microbiology, 144: 1853-1862 (1998) |
| | | pyrophosphokinase | |
| AF041436 | argR | Arginine repressor | |
| AF045998 | impA | Inositol monophosphate phosphatase | |
| AF048764 | argH | Argininosuccinate lyase | |
| AF049897 | argC; argJ; | N-acetylglutamylphosphate reductase; | |
| | argB; argD; | ornithine acetyltransferase; N- | |
| | argF; argR; | acetylglutamate kinase; acetylornithine | |
| | argG; argH | transminase; ornithine | |
| | | carbamoyltransferase; arginine repressor; | |
| | | argininosuccinate synthase; | |
| | | argininosuccinate lyase | |
| AF050109 | inhA | Enoyl-acyl carrier protein reductase | |
| AF050166 | hisG | ATP phosphoribosyltransferase | |
| AF051846 | hisA | Phosphoribosylformimino-5-amino-1- | |
| | | phosphoribosyl-4-imidazolecarboxamide | |
| | | isomerase | |
| AF052652 | metA | Homoserine O-acetyltransferase | Park, S. et al. “Isolation and analysis of metA, a methionine biosynthetic gene |
| | | | encoding homoserine acetyltransferase in Corynebacterium glutamicum,” Mol. |
| | | | Cells., 8(3): 286-294 (1998) |
| AF053071 | aroB | Dehydroquinate synthetase | |
| AF060558 | hisH | Glutamine amidotransferase | |
| AF086704 | hisE | Phosphoribosyl-ATP- | |
| | | pyrophosphohydrolase | |
| AF114233 | aroA | 5-enolpyruvylshikimate 3-phosphate | |
| | | synthase | |
| AF116184 | panD | L-aspartate-alpha-decarboxylase precursor | Dusch, N. et al. “Expression of the Corynebacterium glutamicum panD gene |
| | | | encoding L-aspartate-alpha-decarboxylase leads to pantothenate |
| | | | overproduction in Escherichia coli,” Appl. Environ. Microbiol., |
| | | | 65(4)1530-1539 (1999) |
| AF124518 | aroD; aroE | 3-dehydroquinase; shikimate | |
| | | dehydrogenase | |
| AF124600 | aroC; aroK; | Chorismate synthase; shikimate kinase; 3- | |
| | aroB; pepQ | dehydroquinate synthase; putative | |
| | | cytoplasmic peptidase | |
| AF145897 | inhA | | |
| AF145898 | inhA | | |
| AJ001436 | ectP | Transport of ectoine, glycine betaine, | Peter, H. et al. “Corynebacterium glutamicum is equipped with four secondary |
| | | proline | carriers for compatible solutes: Identification, sequencing, and characterization |
| | | | of the proline/ectoine uptake system, ProP, and the ectoine/proline/glycine |
| | | | betaine carrier, EctP,” J. Bacteriol., 180(22): 6005-6012 (1998) |
| AJ004934 | dapD | Tetrahydrodipicolinate succinylase | Wehrmann, A. et al. “Different modes of diaminopimelate synthesis and their |
| | | (incompleteⁱ) | role in cell wall integrity: A study with Corynebacterium glutamicum,” J. |
| | | | Bacteriol., 180(12): 3159-3165 (1998) |
| AJ007732 | ppc; secG; | Phosphoenolpyruvate-carboxylase; ?; high | |
| | amt; ocd; | affinity ammonium uptake protein; | |
| | soxA | putative ornithine-cyclodecarboxylase; | |
| | | sarcosine oxidase | |
| AJ010319 | ftsY, glnB, | Involved in cell division; PII protein; | Jakoby, M. et al. “Nitrogen regulation in Corynebacterium glutamicum; |
| | glnD; srp; | uridylyltransferase (uridylyl-removing | Isolation of genes involved in biochemical characterization of corresponding |
| | amtP | enzmye); signal recognition particle; low | proteins,” FEMS Microbiol., 173(2): 303-310 (1999) |
| | | affinity ammonium uptake protien | |
| AJ132968 | cat | Chloramphenicol aceteyl transferase | |
| AJ224946 | mqo | L-malate: quinone oxidoreductase | Molenaar, D. et al. “Biochemical and genetic characterization of the |
| | | | membrane-associated malate dehydrogenase (acceptor) from Corynebacterium |
| | | | glutamicum,” Eur. J. Biochem., 254(2): 395-403 (1998) |
| AJ238250 | ndh | NADH dehydrogenase | |
| AJ238703 | porA | Porin | Lichtinger, T. et al. “Biochemical and biophysical characterization of the cell |
| | | | wall porin of Corynebacterium glutamicum: The channel is formed by a low |
| | | | molecular mass polypeptide,” Biochemistry, 37(43): 15024-15032 (1998) |
| D17429 | | Transposable element IS31831 | Vertes, A. A. et al. “Isolation and characterization of IS31831, a transposable |
| | | | element from Corynebacterium glutamicum,” Mol. Microbiol., 11(4): 739-746 |
| | | | (1994) |
| D84102 | odhA | 2-oxoglutarate dehydrogenase | Usuda, Y. et al. “Molecular cloning of the Corynebacterium glutamicum |
| | | | (Brevibacterium lactofermentum AJ12036) odhA gene encoding a novel type |
| | | | of 2-oxoglutarate dehydrogenase,” Microbiology, 142: 3347-3354 (1996) |
| E01358 | hdh; hk | Homoserine dehydrogenase; homoserine | Katsumata, R. et al. “Production of L-thereonine and L-isoleucine,” Patent: JP |
| | | kinase | 1987232392-A 1 Oct. 12, 1987 |
| E01359 | | Upstream of the start codon of homoserine | Katsumata, R. et al. “Production of L-thereonine and L-isoleucine,” Patent: JP |
| | | kinase gene | 1987232392-A 2 Oct. 12, 1987 |
| E01375 | | Tryptophan operon | |
| E01376 | trpL; trpE | Leader peptide; anthranilate synthase | Matsui, K. et al. “Tryptophan operon, peptide and protein coded thereby, |
| | | | utilization of tryptophan operon gene expression and production of |
| | | | tryptophan,” Patent: JP 1987244382-A 1 Oct. 24, 1987 |
| E01377 | | Promoter and operator regions of | Matsui, K. et al. “Tryptophan operon, peptide and protein coded thereby, |
| | | tryptophan operon | utilization of tryptophan operon gene expression and production of |
| | | | tryptophan,” Patent: JP 1987244382-A 1 Oct. 24, 1987 |
| E03937 | | Biotin-synthase | Hatakeyama, K. et al. “DNA fragment containing gene capable of coding |
| | | | biotin synthetase and its utilization,” Patent: JP 1992278088-A 1 Oct. 02, 1992 |
| E04040 | | Diamino pelargonic acid aminotransferase | Kohama, K. et al. ”Gene coding diaminopelargonic acid aminotransferase and |
| | | | desthiobiotin synthetase and its utilization,” Patent: JP 1992330284-A 1 |
| | | | Nov. 18, 1992 |
| E04041 | | Desthiobiotinsynthetase | Kohama, K. et al. “Gene coding diaminopelargonic acid aminotransferase and |
| | | | desthiobiotin synthetase and its utilization,” Patent: JP 1992330284-A 1 |
| | | | Nov. 18, 1992 |
| E04307 | | Flavum aspartase | Kurusu, Y. et al. “Gene DNA coding aspartase and utilization thereof,” Patent: |
| | | | JP 1993030977-A 1 Feb. 09, 1993 |
| E04376 | | Isocitric acid lyase | Katsumata, R. et al. “Gene manifestation controlling DNA,” Patent: JP |
| | | | 1993056782-A 3 Mar. 09, 1993 |
| E04377 | | Isocitric acid lyase N-terminal fragment | Katsumata, R. et al. “Gene manifestation controlling DNA,” Patent: JP |
| | | | 1993056782-A 3 Mar. 09, 1993 |
| E04484 | | Prephenate dehydratase | Sotouchi, N. et al. “Production of L-phenylalanine by fermentation,” Patent: JP |
| | | | 1993076352-A 2 Mar. 30, 1993 |
| E05108 | | Aspartokinase | Fugono, N. et al. “Gene DNA coding Aspartokinase and its use,” Patent: JP |
| | | | 1993184366-A 1 Jul. 27, 1993 |
| E05112 | | Dihydro-dipichorinate synthetase | Hatakeyama, K. et al. “Gene DNA coding dihydrodipicolinic acid synthetase |
| | | | and its use,” Patent: JP 1993184371-A 1 Jul. 27, 1993 |
| E05776 | | Diaminopimelic acid dehydrogenase | Kobayashi, M. et al. “Gene DNA coding Diaminopimelic acid dehydrogenase |
| | | | and its use,” Patent: JP 1993284970-A 1 Nov. 02, 1993 |
| E05779 | | Threonine synthase | Kohama, K. et al. “Gene DNA coding threonine synthase and its use,” Patent: |
| | | | JP 1993284972-A 1 Nov. 02, 1993 |
| E06110 | | Prephenate dehydratase | Kikuchi, T. et al. “Production of L-phenylalanine by fermentation method,” |
| | | | Patent: JP 1993344881-A 1 Dec. 27, 1993 |
| E06111 | | Mutated Prephenate dehydratase | Kikuchi, T. et al. “Production of L-phenylalanine by fermentation method,” |
| | | | Patent: JP 1993344881-A 1 Dec. 27, 1993 |
| E06146 | | Acetohydroxy acid synthetase | Inui, M. et al. “Gene capable of coding Acetohydroxy acid synthetase and its |
| | | | use,” Patent: JP 1993344893-A 1 Dec. 27, 1993 |
| E06825 | | Aspartokinase | Sugimoto, M. et al. “Mutant aspartokinase gene,” patent: JP 1994062866-A 1 |
| | | | Mar. 08, 1994 |
| E06826 | | Mutated aspartokinase alpha subunit | Sugimoto, M. et al. “Mutant aspartokinase gene,” patent: JP 1994062866-A 1 |
| | | | Mar. 08, 1994 |
| E06827 | | Mutated aspartokinase alpha subunit | Sugimoto, M. et al. “Mutant aspartokinase gene,” patent: JP 1994062866-A 1 |
| | | | Mar. 08, 1994 |
| E07701 | secY | | Honno, N. et al. “Gene DNA participating in integration of membraneous |
| | | | protein to membrane,” Patent: JP 1994169780-A 1 Jun. 21, 1994 |
| E08177 | | Aspartokinase | Sato, Y. et al. “Genetic DNA capable of coding Aspartokinase released from |
| | | | feedback inhibition and its utilization,” Patent: JP 1994261766-A 1 |
| | | | Sep. 20, 1994 |
| E08178, | | Feedback inhibition-released | Sato, Y. et al. “Genetic DNA capable of coding Aspartokinase released from |
| E08179, | | Aspartokinase | feedback inhibition and its utilization,” Patent: JP 1994261766-A 1 |
| E08180, | | | Sep. 20, 1994 |
| E08181, | | | |
| E08182 | | | |
| E08232 | | Acetohydroxy-acid isomeroreductase | Inui, M. et al. “Gene DNA coding acetohydroxy acid isomeroreductase,” |
| | | | Patent: JP 1994277067-A 1 Oct. 04, 1994 |
| E08234 | secE | | Asai, Y. et al. “Gene DNA coding for translocation machinery of protein,” |
| | | | Patent: JP 1994277073-A 1 Oct. 04, 1994 |
| E08643 | | FT aminotransferase and desthiobiotin | Hatakeyama, K. et al. “DNA fragment having promoter function in |
| | | synthetase promoter region | coryneform bacterium,” Patent: JP 1995031476-A 1 Feb. 03, 1995 |
| E08646 | | Biotin synthetase | Hatakeyama, K. et al. “DNA fragment having promoter function in |
| | | | coryneform bacterium,” Patent: JP 1995031476-A 1 Feb. 03, 1995 |
| E08649 | | Aspartase | Kohama, K. et al “DNA fragment having promoter function in coryneform |
| | | | bacterium,” Patent: JP 1995031478-A 1 Feb. 03, 1995 |
| E08900 | | Dihydrodipicolinate reductase | Madori, M. et al. “DNA fragment containing gene coding Dihydrodipicolinate |
| | | | acid reductase and utilization thereof,” Patent: JP 1995075578-A 1 |
| | | | Mar. 20, 1995 |
| E08901 | | Diaminopimelic acid decarboxylase | Madori, M. et al. “DNA fragment containing gene coding Diaminopimelic acid |
| | | | decarboxylase and utilization thereof,” Patent: JP 1995075579-A 1 |
| | | | Mar. 20, 1995 |
| E12594 | | Serine hydroxymethyltransferase | Hatakeyama, K. et al. “Production of L-trypophan,” Patent: JP 1997028391-A |
| | | | 1 Feb. 04, 1997 |
| E12760, | | transposase | Moriya, M. et al. “Amplification of gene using artificial transposon,” Patent: |
| E12759, | | | JP 1997070291-A Mar. 18, 1997 |
| E12758 | | | |
| E12764 | | Arginyl-tRNA synthetase; diaminopimelic | Moriya, M. et al. “Amplification of gene using artificial transposon,” Patent: |
| | | acid decarboxylase | JP 1997070291-A Mar. 18, 1997 |
| E12767 | | Dihydrodipicolinic acid synthetase | Moriya, M. et al. “Amplification of gene using artificial transposon,” Patent: |
| | | | JP 1997070291-A Mar. 18, 1997 |
| E12770 | | aspartokinase | Moriya, M. et al. “Amplification of gene using artificial transposon,” Patent: |
| | | | JP 1997070291-A Mar. 18, 1997 |
| E12773 | | Dihydrodipicolinic acid reductase | Moriya, M. et al. “Amplification of gene using artificial transposon,” Patent: |
| | | | JP 1997070291-A Mar. 18, 1997 |
| E13655 | | Glucose-6-phosphate dehydrogenase | Hatakeyama, K. et al. “Glucose-6-phosphate dehydrogenase and DNA capable |
| | | | of coding the same,” Patent: JP 1997224661-A 1 Sep. 02, 1997 |
| L01508 | IlvA | Threonine dehydratase | Moeckel, B. et al. “Functional and structural analysis of the threonine |
| | | | dehydratase of Corynebacterium glutamicum,” J. Bacteriol., 174: 8065-8072 |
| | | | (1992) |
| L07603 | EC 4.2.1.15 | 3-deoxy-D-arabinoheptulosonate-7- | Chen, C. et al. “The cloning and nucleotide sequence of Corynebacterium |
| | | phosphate synthase | glutamicum 3-deoxy-D-arabinoheptulosonate-7-phosphate synthase gene,” |
| | | | FEMS Microbiol. Lett., 107: 223-230 (1993) |
| L09232 | IlvB; ilvN; | Acetohydroxy acid synthase large subunit; | Keilhauer, C. et al. “Isoleucine synthesis in Corynebacterium glutamicum: |
| | ilvC | Acetohydroxy acid synthase small subunit; | molecular analysis of the ilvB-ilvN-ilvC operon,” J. Bacteriol., 175(17): |
| | | Acetohydroxy acid isomeroreductase | 5595-5603 (1993) |
| L18874 | PtsM | Phosphoenolpyruvate sugar | Fouet, A et al. “Bacillus subtilis sucrose-specific enzyme II of the |
| | | phosphotransferase | phosphotransferase system: expression in Escherichia coli and homology to |
| | | | enzymes II from enteric bacteria,” PNAS USA, 84(24): 8773-8777 (1987); |
| | | | Lee, J. K. et al. “Nucleotide sequence of the gene encoding the |
| | | | Corynebacterium glutamicum mannose enzyme II and analyses of the deduced |
| | | | protein sequence,” FEMS Microbiol. Lett., 119(1-2): 137-145 (1994) |
| L27123 | aceB | Malate synthase | Lee, H-S. et al. “Molecular characterization of aceB, a gene encoding malate |
| | | | synthase in Corynebacterium glutamicum,” J. Microbiol. Biotechnol., |
| | | | 4(4): 256-263 (1994) |
| L27126 | | Pyruvate kinase | Jetten, M. S. et al. “Structural and functional analysis of pyruvate kinase from |
| | | | Corynebacterium glutamicum,” Appl. Environ. Microbiol., 60(7): 2501-2507 |
| | | | (1994) |
| L28760 | aceA | Isocitrate lyase | |
| L35906 | dtxr | Diphtheria toxin repressor | Oguiza, J. A. et al. “Molecular cloning, DNA sequence analysis, and |
| | | | characterization of the Corynebacterium diphtheriae dtxR from Brevibacterium |
| | | | lactofermentum,” J. Bacteriol., 177(2): 465-467 (1995) |
| M13774 | | Prephenate dehydratase | Follettie, M. T. et al. “Molecular cloning and nucleotide sequence of the |
| | | | Corynebacterium glutamicum pheA gene,” J. Bacteriol., 167: 695-702 (1986) |
| M16175 | 5S rRNA | | Park, Y-H. et al. “Phylogenetic analysis of the coryneform bacteria by 56 |
| | | | rRNA sequences,” J. Bacteriol., 169: 1801-1806 (1987) |
| M16663 | trpE | Anthranilate synthase, 5′ end | Sano, K. et al. “Structure and function of the trp operon control regions of |
| | | | Brevibacterium lactofermentum, a glutamic-acid-producing bacterium,” Gene, |
| | | | 52: 191-200 (1987) |
| M16664 | trpA | Tryptophan synthase, 3′ end | Sano, K. et al. “Structure and function of the trp operon control regions of |
| | | | Brevibacterium lactofermentum, a glutamic-acid-producing bacterium,” Gene, |
| | | | 52: 191-200 (1987) |
| M25819 | | Phosphoenolpyruvate carboxylase | O'Regan, M. et al. “Cloning and nucleotide sequence of the |
| | | | Phosphoenolpyruvate carboxylase-coding gene of Corynebacterium |
| | | | glutamicum ATCC13032,” Gene, 77(2): 237-251 (1989) |
| M85106 | | 23S rRNA gene insertion sequence | Roller, C. et al. “Gram-positive bacteria with a high DNA G + C content are |
| | | | characterized by a common insertion within their 23S rRNA genes,” J. Gen. |
| | | | Microbiol., 138: 1167-1175 (1992) |
| M85107, | | 23S rRNA gene insertion sequence | Roller, C. et al. “Gram-positive bacteria with a high DNA G + C content are |
| M85108 | | | characterized by a common insertion within their 23S rRNA genes,” J. Gen. |
| | | | Microbiol., 138: 1167-1175 (1992) |
| M89931 | aecD; brnQ; | Beta C-S lyase; branched-chain amino | Rossol, I. et al. “The Corynebacterium glutamicum aecD gene encodes a C-S |
| | yhbw | acid uptake carrier; hypothetical | lyase with alpha, beta-elimination activity that degrades aminoethylcysteine,” |
| | | protein yhbw | J. Bacteriol., 174(9): 2968-2977 (1992); Tauch, A. et al. “Isoleucine uptake in |
| | | | Corynebacterium glutamicum ATCC 13032 is directed by the brnQ gene |
| | | | product,” Arch. Microbiol., 169(4): 303-312 (1998) |
| S59299 | trp | Leader gene (promoter) | Herry, D. M. et al. “Cloning of the trp gene cluster from a tryptophan- |
| | | | hyperproducing strain of Corynebacterium glutamicum: identification of a |
| | | | mutation in the trp leader sequence,” Appl. Environ. Microbiol., 59(3): |
| | | | 791-799 (1993) |
| U11545 | trpD | Anthranilate phosphoribosyltransferase | O'Gara, J. P. and Dunican, L. K. (1994) Complete nucleotide sequence of the |
| | | | Corynebacterium glutamicum ATCC 21850 tpD gene.” Thesis, Microbiology |
| | | | Department, University College Galway, Ireland. |
| U13922 | cglIM; | Putative type II 5-cytosoine | Schafer, A. et al. “Cloning and characterization of a DNA region encoding a |
| | cglIR; clgIIR | methyltransferase; putative type II | stress-sensitive restriction system from Corynebacterium glutamicum ATCC |
| | | restriction endonuclease; putative type I or | 13032 and analysis of its role in intergeneric conjugation with Escherichia |
| | | type III restriction endonuclease | coli,” J. Bacteriol., 176(23): 7309-7319 (1994); Schafer, A. et al. “The |
| | | | Corynebacterium glutamicum cglIM gene encoding a 5-cytosine in an McrBC- |
| | | | deficient Escherichia coli strain,” Gene, 203(2): 95-101 (1997) |
| U14965 | recA | | |
| U31224 | ppx | | Ankri, S. et al. “Mutations in the Corynebacterium glutamicumproline |
| | | | biosynthetic pathway: A natural bypass of the proA step,” J. Bacteriol., |
| | | | 178(15): 4412-4419 (1996) |
| U31225 | proC | L-proline: NADP+ 5-oxidoreductase | Ankri, S. et al. “Mutations in the Corynebacterium glutamicumproline |
| | | | biosynthetic pathway: A natural bypass of the proA step,” J. Bacteriol., |
| | | | 178(15): 4412-4419 (1996) |
| U31230 | obg; proB; | ?; gamma glutamyl kinase; similar to D- | Ankri, S. et al. “Mutations in the Corynebacterium glutamicumproline |
| | unkdh | isomer specific 2-hydroxyacid | biosynthetic pathway: A natural bypass of the proA step,” J. Bacteriol., |
| | | dehydrogenases | 178(15): 4412-4419 (1996) |
| U31281 | bioB | Biotin synthase | Serebriiskii, I. G., “Two new members of the bio B superfamily: Cloning, |
| | | | sequencing and expression of bio B genes of Methylobacillus flagellatum and |
| | | | Corynebacterium glutamicum,” Gene, 175: 15-22 (1996) |
| U35023 | thtR; accBC | Thiosulfate sulfurtransferase; acyl CoA | Jager, W. et al. “A Corynebacterium glutamicum gene encoding a two-domain |
| | | carboxylase | protein similar to biotin carboxylases and biotin-carboxyl-carrier proteins,” |
| | | | Arch. Microbiol., 166(2); 76-82 (1996) |
| U43535 | cmr | Multidrug resistance protein | Jager, W. et al. “A Corynebacterium glutamicum gene conferring multidrug |
| | | | resistance in the heterologous host Escherichia coli,” J. Bacteriol., |
| | | | 179(7): 2449-2451 (1997) |
| U43536 | clpB | Heat shock ATP-binding protein | |
| U53587 | aphA-3 | 3′5″-aminoglycoside phosphotransferase | |
| U89648 | | Corynebacterium glutamicum unidentified | |
| | | sequence involved in histidine | |
| | | biosynthesis, partial sequence | |
| X04960 | trpA; trpB; | Tryptophan operon | Matsui, K. et al. “Complete nucleotide and deduced amino acid sequences of |
| | trpC; trpD; | | the Brevibacterium lactofermentum tryptophan operon,” Nucleic Acids Res., |
| | trpE; trpG; | | 14(24): 10113-10114 (1986) |
| | trpL | | |
| X07563 | lys A | DAP decarboxylase (meso- | Yeh, P. et al. “Nucleic sequence of the lysA gene of Corynebacterium |
| | | diaminopimelate decarboxylase, | glutamicum and possible mechanisms for modulation of its expression,” Mol. |
| | | EC 4.1.1.20) | Gen. Genet., 212(1): 112-119 (1988) |
| X14234 | EC 4.1.1.31 | Phosphoenolpyruvate carboxylase | Eikmanns, B. J. et al. “The Phosphoenolpyruvate carboxylase gene of |
| | | | Corynebacterium glutamicum: Molecular cloning, nucleotide sequence, and |
| | | | expression,” Mol. Gen. Genet., 218(2): 330-339 (1989); Lepiniec, L. et al. |
| | | | “Sorghum Phosphoenolpyruvate carboxylase gene family: structure, function |
| | | | and molecular evolution,” Plant. Mol. Biol., 21 (3): 487-502 (1993) |
| X17313 | fda | Fructose-bisphosphate aldolase | Von der Osten, C. H. et al. “Molecular cloning, nucleotide sequence and fine- |
| | | | structural analysis of the Corynebacterium glutamicum fda gene: structural |
| | | | comparison of C. glutamicum fructose-1, 6-biphosphate aldolase to class I and |
| | | | class II aldolases,” Mol. Microbiol., |
| X53993 | dapA | L-2, 3-dihydrodipicolinate synthetase (EC | Bonnassie, S. et al. “Nucleic sequence of the dapA gene from |
| | | 4.2.1.52) | Corynebacterium glutamicum,” Nucleic Acids Res., 18(21): 6421 (1990) |
| X54223 | | AttB-related site | Cianciotto, N. et al. “DNA sequence homology between att B-related sites of |
| | | | Corynebacterium diphtheriae, Corynebacterium ulcerans, Corynebacterium |
| | | | glutamicum, and the attP site of lambdacorynephage,” FEMS. Microbiol, |
| | | | Lett., 66: 299-302 (1990) |
| X54740 | argS; lysA | Arginyl-tRNA synthetase; | Marcel, T. et al. “Nucleotide sequence and organization of the upstream region |
| | | Diaminopimelate decarboxylase | of the Corynebacterium glutamicum lysA gene,” Mol. Microbiol., 4(11): |
| | | | 1819-1830 (1990) |
| X55994 | trpL; trpE | Putative leader peptide; anthranilate | Heery, D. M. et al. “Nucleotide sequence of the Corynebacterium glutamicum |
| | | synthase component 1 | trpE gene,” Nucleic Acids Res., 18(23): 7138 (1990) |
| X56037 | thrC | Threonine synthase | Han, K. S. et al. “The molecular structure of the Corynebacterium glutamicum |
| | | | threonine synthase gene,” Mol. Microbiol., 4(10): 1693-1702 (1990) |
| X56075 | attB-related | Attachment site | Cianciotto, N. et al. “DNA sequence homology between att B-related sites of |
| | site | | Corynebacterium diphtheriae, Corynebacterium ulcerans, Corynebacterium |
| | | | glutamicum, and the attP site of lambdacorynephage,” FEMS. Microbiol, |
| | | | Lett., 66: 299-302 (1990) |
| X57226 | lysC-alpha; | Aspartokinase-alpha subunit; | Kalinowski, J. et al. “Genetic and biochemical analysis of the Aspartokinase |
| | lysC-beta; | Aspartokinase-beta subunit; aspartate beta | from Corynebacterium glutamicum,” Mol. Microbiol., 5(5): 1197-1204 (1991); |
| | asd | semialdehyde dehydrogenase | Kalinowski, J. et al. “Aspartokinase genes lysC alpha and lysC beta overlap |
| | | | and are adjacent to the aspertate beta-semialdehyde dehydrogenase gene asd in |
| | | | Corynebacterium glutamicum,” Mol. Gen. Genet., 224(3): 317-324 (1990) |
| X59403 | gap; pgk; tpi | Glyceraldehyde-3-phosphate; | Eikmanns, B. J. “Identification, sequence analysis, and expression of a |
| | | phosphoglycerate kinase; triosephosphate | Corynebacterium glutamicum gene cluster encoding the three glycolytic |
| | | isomerase | enzymes glyceraldehyde-3-phosphate dehydrogenase, 3-phosphoglycerate |
| | | | kinase, and triosephosphate isomeras,” J. Bacteriol., 174(19): 6076-6086 |
| | | | (1992) |
| X59404 | gdh | Glutamate dehydrogenase | Bormann, E. R. et al. “Molecular analysis of the Corynebacterium glutamicum |
| | | | gdh gene encoding glutamate dehydrogenase,” Mol. Microbiol., 6(3): 317-326 |
| | | | (1992) |
| X60312 | lysI | L-lysine permease | Seep-Feldhaus, A. H. et al. “Molecular analysis of the Corynebacterium |
| | | | glutamicum lysl gene involved in lysine uptake,” Mol. Microbiol., 5(12): |
| | | | 2995-3005 (1991) |
| X66078 | cop1 | Ps1 protein | Joliff, G. et al. “Cloning and nucleotide sequence of the csp1 gene encoding |
| | | | PS1, one of the two major secreted proteins of Corynebacterium glutamicum: |
| | | | The deduced N-terminal region of PS1 is similar to the Mycobacterium antigen |
| | | | 85 complex,” Mol. Microbial., 6(16): 2349-2362 (1992) |
| X66112 | glt | Citrate synthase | Eikmanns, B. J. et al. “Cloning sequence, expression and transcriptional |
| | | | analysis of the Corynebacterium glutamicum gltA gene encoding citrate |
| | | | synthase,” Microbiol., 140: 1817-1828 (1994) |
| X67737 | dapB | Dihydrodipicolinate reductase | |
| X69103 | csp2 | Surface layer protein PS2 | Peyret, J. L. et al. “Characterization of the cspB gene encoding PS2, an ordered |
| | | | surface-layer protein in Corynebacterium glutamicum,” Mol. Microbiol., |
| | | | 9(1): 97-109 (1993) |
| X69104 | | IS3 related insertion element | Bonamy, C. et al. “Identification of IS1206, a Corynebacterium glutamicum |
| | | | IS3-related insertion sequence and phylogenetic analysis,” Mol. Microbial., |
| | | | 14(3): 571-581 (1994) |
| X70959 | leuA | Isopropylmalate synthase | Patek, M. et al. “Leucine synthesis in Corynebacterium glutamicum: enzyme |
| | | | activities, structure of leuA, and effect of leuA inactivation on lysine |
| | | | synthesis,” Appl. Environ. Microbiol., 60(1): 133-140 (1994) |
| X71489 | icd | Isocitrate dehydrogenase (NADP+) | Eikmanns, B. J. et al. “Cloning sequence analysis, expression, and inactivation |
| | | | of the Corynebacterium glutamicum icd gene encoding isocitrate |
| | | | dehydrogenase and biochemical characterization of the enzyme,” J. Bacteriol., |
| | | | 177(3): 774-782 (1995) |
| X72855 | GDHA | Glutamate dehydrogenase (NADP+) | |
| X75083, | mtrA | 5-methyltryptophan resistance | Heery, D. M. et al. “A sequence from a tryptophan-hyperproducing strain of |
| X70584 | | | Corynebacterium glutamicum encoding resistance to 5-methyltryptophan,” |
| | | | Biochem. Biophys. Res. Commun., 201(3): 1255-1262 (1994) |
| X75085 | recA | | Fitzpatrick, R. et al. “Construction and characterization of recA mutant strains |
| | | | of Corynebacterium glutamicum and Brevibacterium lactofermentum,” Appl. |
| | | | Microbiol. Biotechnol., 42(4): 575-580 (1994) |
| X75504 | aceA; thiX | Partial Isocitrate lyase; ? | Reinscheid, D. J. et al. “Characterization of the isocitrate lyase gene from |
| | | | Corynebacterium glutamicum and biochemical analysis of the enzyme,” J. |
| | | | Bacteriol., 176(12): 3474-3483 (1994) |
| X76875 | | ATPase beta-subunit | Ludwig, W. et al. “Phylogenetic relationships of bacteria based on comparative |
| | | | sequence analysis of elongation factor Tu and ATP-synthase beta-subunit |
| | | | genes,” Antonie Van Leeuwenhoek, 64: 285-305 (1993) |
| X77034 | tuf | Elongation factor Tu | Ludwig, W. et al. “Phylogenetic relationships of bacteria based on comparative |
| | | | sequence analysis of elongation factor Tu and ATP-synthase beta-subunit |
| | | | genes,” Antonie Van Leeuwenhoek, 64: 285-305 (1993) |
| X77384 | recA | | Billman-Jacobe, H. “Nucleotide sequence of a recA gene from |
| | | | Corynebacterium glutamicum,” DNA Seq., 4(6): 403-404 (1994) |
| X78491 | aceB | Malate synthase | Reinscheid, D. J. et al. “Malate synthase from Corynebacterium glutamicum |
| | | | pta-ack operon encoding phosphotransacetylase: sequence analysis,” |
| | | | Microbiology, 140: 3099-3108 (1994) |
| X80629 | 16S rDNA | 16S ribosomal RNA | Rainey, F. A. et al. “Phylogenetic analysis of the genera Rhodococcus and |
| | | | Norcardia and evidence for the evolutionary origin of the genus Norcardia |
| | | | from within the radiation of Rhodococcus species,” Microbiol., 141: 523-528 |
| | | | (1995) |
| X81191 | gluA; gluB; | Glutamate uptake system | Kronemeyer, W. et al. “Structure of the gluABCD cluster encoding the |
| | gluC; gluD | | glutamate uptake system of Corynebacterium glutamicum,” J. Bacteriol., |
| | | | 177(5): 1152-1158 (1995) |
| X81379 | dapE | Succinyldiaminopimelate desuccinylase | Wehrmann, A. et al. “Analysis of different DNA fragments of |
| | | | Corynebacterium glutamicum complementing dapE of Escherichia coli,” |
| | | | Microbiology, 40: 3349-56 (1994) |
| X82061 | 16S rDNA | 165 ribosomal RNA | Ruimy, R. et al. “Phylogeny of the genus Corynebacterium deduced from |
| | | | analyses of small-subunit ribosomal DNA sequences,” Int. J. Syst. Bacteriol., |
| | | | 45(4): 740-746 (1995) |
| X82928 | asd; lysC | Aspartate-semialdehyde dehydrogenase; ? | Serebrijski, I. et al. “Multicopy suppression by asd gene and osmotic stress- |
| | | | dependent complementation by heterologous proA in proA mutants,” J. |
| | | | Bacteriol., 177(24): 7255-7260 (1995) |
| X82929 | proA | Gamma-glutamyl phosphate reductase | Serebrijski, I. et al. “Multicopy suppression by asd gene and osmotic stress- |
| | | | dependent complementation by heterologous proA in proA mutants,” J. |
| | | | Bacteriol., 177(24): 7255-7260 (1995) |
| X84257 | 16S rDNA | 16S ribosomal RNA | Pascual, C. et al. “Phylogenetic analysis of the genus Corynebacterium based |
| | | | on 16S rRNA gene sequences,” Int. J. Syst. Bacteriol., 45(4): 724-728 (1995) |
| X85965 | aroP; dapE | Aromatic amino acid permease; ? | Wehrmann, A. et al. “Functional analysis of sequences adjacent to dapE of |
| | | | Corynebacterium glutamicumproline reveals the presence of aroP, which |
| | | | encodes the aromatic amino acid transporter,” J. Bacteriol., 177(20): |
| | | | 5991-5993 (1995) |
| X86157 | argB; argC; | Acetylglutamate kinase; N-acetyl-gamma- | Sakanyan, V. et al. “Genes and enzymes of the acetyl cycle of arginine |
| | argD; argF; | glutamyl-phosphate reductase; | biosynthesis in Corynebacterium glutamicum: enzyme evolution in the early |
| | argJ | acetylornithine aminotransferase; ornithine | steps of the arginine pathway,” Microbiology, 142: 99-108 (1996) |
| | | carbamoyltransferase; glutamate N- | |
| | | acetyltransferase | |
| X89084 | pta; ackA | Phosphate acetyltransferase; acetate kinase | Reinscheid, D. J. et al. “Cloning, sequence analysis, expression and |
| | | | inactivation of the Corynebacterium glutamicum pta-ack operon encoding |
| | | | phosphotransacetylase and acetate kinase,” Microbiology, 145: 503-513 (1999) |
| X89850 | attB | Attachment site | Le Marrec, C. et al. “Genetic characterization of site-specific integration |
| | | | functions of phi AAU2 infecting “Arthrobacter aureus C70,” J. Bacteriol., |
| | | | 178(7): 1996-2004 (1996) |
| X90356 | | Promoter fragment F1 | Patek, M. et al. “Promoters from Corynebacterium glutamicum: cloning, |
| | | | molecular analysis and search for a consensus motif,” Microbiology, |
| | | | 142: 1297-1309 (1996) |
| X90357 | | Promoter fragment F2 | Patek, M. et al. “Promoters from Corynebacterium glutamicum: cloning, |
| | | | molecular analysis and search for a consensus motif,” Microbiology, |
| | | | 142: 1297-1309 (1996) |
| X90358 | | Promoter fragment F10 | Patek, M. et al. “Promoters from Corynebacterium glutamicum: cloning, |
| | | | molecular analysis and search for a consensus motif,” Microbiology, |
| | | | 142: 1297-1309 (1996) |
| X90359 | | Promoter fragment F13 | Patek, M. et al. “Promoters from Corynebacterium glutamicum: cloning, |
| | | | molecular analysis and search for a consensus motif,” Microbiology, |
| | | | 142: 1297-1309 (1996) |
| X90360 | | Promoter fragment F22 | Patek, M. et al. “Promoters from Corynebacterium glutamicum: cloning, |
| | | | molecular analysis and search for a consensus motif,” Microbiology, |
| | | | 142: 1297-1309 (1996) |
| X90361 | | Promoter fragment F34 | Patek, M. et al. “Promoters from Corynebacterium glutamicum: cloning, |
| | | | molecular analysis and search for a consensus motif,” Microbiology, |
| | | | 142: 1297-1309 (1996) |
| X90362 | | Promoter fragment F37 | Patek, M. et al. “Promoters from Corynebacterium glutamicum: cloning, |
| | | | molecular analysis and search for a consensus motif,” Microbiology, |
| | | | 142: 1297-1309 (1996) |
| X90363 | | Promoter fragment F45 | Patek, M. et al. “Promoters from Corynebacterium glutamicum: cloning, |
| | | | molecular analysis and search for a consensus motif,” Microbiology, |
| | | | 142: 1297-1309 (1996) |
| X90364 | | Promoter fragment F64 | Patek, M. et al. “Promoters from Corynebacterium glutamicum: cloning, |
| | | | molecular analysis and search for a consensus motif,” Microbiology, |
| | | | 142: 1297-1309 (1996) |
| X90365 | | Promoter fragment F75 | Patek, M. et al. “Promoters from Corynebacterium glutamicum: cloning, |
| | | | molecular analysis and search for a consensus motif,” Microbiology, |
| | | | 142: 1297-1309 (1996) |
| X90366 | | Promoter fragment PF101 | Patek, M. et al. “Promoters from Corynebacterium glutamicum: cloning, |
| | | | molecular analysis and search for a consensus motif,” Microbiology, |
| | | | 142: 1297-1309 (1996) |
| X90367 | | Promoter fragment PF104 | Patek, M. et al. “Promoters from Corynebacterium glutamicum: cloning, |
| | | | molecular analysis and search for a consensus motif,” Microbiology, |
| | | | 142: 1297-1309 (1996) |
| X90368 | | Promoter fragment PF109 | Patek, M. et al. “Promoters from Corynebacterium glutamicum: cloning, |
| | | | molecular analysis and search for a consensus motif,” Microbiology, |
| | | | 142: 1297-1309 (1996) |
| X93513 | amt | Ammonium transport system | Siewe, R. M. et al. “Functional and genetic characterization of the (methyl) |
| | | | ammonium uptake carrier of Corynebacterium glutamicum,” J. Biol. Chem., |
| | | | 271(10): 5398-5403 (1996) |
| X93514 | betP | Glycine betaine transport system | Peter, H. et al. “Isolation, characterization, and expression of the |
| | | | Corynebacterium glutamicum betP gene, encoding the transport system for the |
| | | | compatible solute glycine betaine,” J. Bacteriol., 178(17): 5229-5234 (1996) |
| X95649 | orf4 | | Patek, M. et al. “Identification and transcriptional analysis of the dapB-ORF2- |
| | | | dapA-ORF4 operon of Corynebacterium glutamicum, encoding two enzymes |
| | | | involved in L-lysine synthesis,” Biotechnol. Lett., 19: 1113-1117 (1997) |
| X96471 | lysE; lysG | Lysine exporter protein; Lysine export | Vrljic, M. et al. “A new type of transporter with a new type of cellular |
| | | regulator protein | function: L-lysine export from Corynebacterium glutamicum,” Mol. |
| | | | Microbiol., 22(5): 815-826 (1996) |
| X96580 | panB; panC; | 3-methyl-2-oxobutanoate | Sahm, H. et al. “D-pantothenate synthesis in Corynebacterium glutamicum and |
| | xylB | hydroxymethyltransferase; pantoate-beta- | use of panBC and genes encoding L-valine synthesis for D-pantothenate |
| | | alanine ligase; xylulokinase | overproduction,” Appl. Environ. Microbiol., 65(5): 1973-1979 (1999) |
| X96962 | | Insertion sequence IS1207 and transposase | |
| X99289 | | Elongation factor P | Ramos, A. et al. “Cloning, sequencing and expression of the gene encoding |
| | | | elongation factor P in the amino-acid producer Brevibacterium lactofermentum |
| | | | (Corynebacterium glutamicum ATCC 13869),” Gene, 198: 217-222 (1997) |
| Y00140 | thrB | Homoserine kinase | Mateos, L. M. et al. “Nucleotide sequence of the homoserine kinase (thrB) |
| | | | gene of the Brevibacterium lactofermentum,” Nucleic Acids Res., 15(9): |
| | | | 3922 (1987) |
| Y00151 | ddh | Meso-diaminopimelate D-dehydrogenase | Ishino, S. et al. “Nucleotide sequence of the meso-diaminopimelate D- |
| | | (EC 1.4.1.16) | dehydrogenase gene from Corynebacterium glutamicum,” Nucleic Acids Res., |
| | | | 15(9): 3917 (1987) |
| Y00476 | thrA | Homoserine dehydrogenase | Mateos, L. M. et al. “Nucleotide sequence of the homoserine dehydrogenase |
| | | | (thrA) gene of the Brevibacterium lactofermentum,” Nucleic Acids Res., |
| | | | 15(24): 10598 (1987) |
| Y00546 | hom; thrB | Homoserine dehydrogenase; homoserine | Peoples, O. P. et al. “Nucleotide sequence and fine structural analysis of the |
| | | kinase | Corynebacterium glutamicum hom-thrB operon,” Mol. Microbiol., 2(1): |
| | | | 63-72 (1988) |
| Y08964 | murC; ftsQ/ | UPD-N-acetylmuramate-alanine ligase; | Honrubia, M. P. et al. “Identification, characterization, and chromosomal |
| | divD; ftsZ | division initiation protein or cell division | organization of the ftsZ gene from Brevibacterium lactofermentum,” Mol. Gen. |
| | | protein; cell division protein | Genet., 259(1): 97-104 (1998) |
| Y09163 | putP | High affinity proline transport system | Peter, H. et al. “Isolation of the putP gene of Corynebacterium |
| | | | glutamicumproline and characterization of a low-affinity uptake system for |
| | | | compatible solutes,” Arch. Microbiol., 168(2): 143-15 (1997) |
| Y09548 | pyc | Pyruvate carboxylase | Peters-Wendisch, P. G. et al. “Pyruvate carboxylase from Corynebacterium |
| | | | glutamicum: characterization, expression and inactivation of the pyc gene,” |
| | | | Microbiology, 144: 915-927 (1998) |
| Y09578 | leuB | 3-isopropylmalate dehydrogenase | Patek, M. et al. “Analysis of the leuB gene from Corynebacterium |
| | | | glutamicum,” Appl. Microbiol. Biotechnol., 50(1): 42-47 (1998) |
| Y12472 | | Attachment site bacteriophage Phi-16 | Moreau, S. et al. “Site-specific integration of corynephage Phi-16: The |
| | | | construction of an integration vector,” Microbiol., 145: 539-548 (1999) |
| Y12537 | proP | Proline/ectoine uptake system protein | Peter, H. et al. “Corynebacterium glutamicum is equipped with four secondary |
| | | | carriers for compatible solutes: Identification, sequencing, and characterization |
| | | | of the proline/ectoine uptake system, ProP, and the ectoine/proline/glycine |
| | | | betaine carrier, EctP,” J. Bacteriol., 180(22): 6005-6012 (1998) |
| Y13221 | glnA | Glutamine synthetase I | Jakoby, M. et al. “Isolation of Corynebacterium glutamicum glnA gene |
| | | | encoding glutamine synthetase I,” FEMS Microbiol. Lett., 154(1): |
| | | | 81-88 (1997) |
| Y16642 | lpd | Dihydrolipoamide dehydrogenase | |
| Y18059 | | Attachment site Corynephage 304L | Moreau, S. et al. “Analysis of the integration functions of &phi;304L: An |
| | | | integrase module among corynephages,” Virology, 255(1): 150-159 (1999) |
| Z21501 | argS; lysA | Arginyl-tRNA synthetase; | Oguiza, J. A. et al. “A gene encoding arginyl-tRNA synthetase is located in the |
| | | diaminopimelate decarboxylase (partial) | upstream region of the lysA gene in Brevibacterium lactofermentum: |
| | | | Regulation of argS-lysA cluster expression by arginine,” J. Bacteriol., |
| | | | 175(22): 7356-7362 (1993) |
| Z21502 | dapA; dapB | Dihydrodipicolinate synthase; | Pisabarro, A. et al. “A cluster of three genes (dapA, orf2, and dapB) of |
| | | dihydrodipicolinate reductase | Brevibacterium lactofermentum encodes dihydrodipicolinate reductase, and a |
| | | | third polypeptide of unknown function,” J. Bacteriol., 175(9): 2743-2749 |
| | | | (1993) |
| Z29563 | thrC | Threonine synthase | Malumbres, M. et al. “Analysis and expression of the thrC gene of the encoded |
| | | | threonine synthase,” Appl. Environ. Microbiol., 60(7)2209-2219 (1994) |
| Z46753 | 16S rDNA | Gene for 16S ribosomal RNA | |
| Z49822 | sigA | SigA sigma factor | Oguiza, J. A. et al “Multiple sigma factor genes in Brevibacterium |
| | | | lactofermentum: Characterization of sigA and sigB,” J. Bacteriol., 178(2): |
| | | | 550-553 (1996) |
| Z49823 | galE; dtxR | Catalytic activity UDP-galactose 4- | Oguiza, J. A. et al “The galE gene encoding the UDP-galactose 4-epimerase of |
| | | epimerase; diphtheria toxin regulatory | Brevibacterium lactofermentum is coupled transcriptionally to the dmdR |
| | | protein | gene,” Gene, 177: 103-107 (1996) |
| Z49824 | orf1; sigB | ?; SigB sigma factor | Oguiza, J. A. et al “Multiple sigma factor genes in Brevibacterium |
| | | | lactofermentum: Characterization of sigA and sigB,” J. Bacteriol., 178(2): |
| | | | 550-553 (1996) |
| Z66534 | | Transposase | Correia, A. et al. “Cloning and characterization of an IS-like element present in |
| | | | the genome of Brevibacterium lactofermentum ATCC 13869,” Gene, |
| | | | 170(1): 91-94 (1996) |

¹A sequence for this gene was published in the indicated reference. However, the sequence obtained by the inventors of the present application is significantly longer than the published version. It is believed that the published version relied on an incorrect start codon, and thus represents only a fragment of the actual coding region.

TABLE 1
GENES IN THE APPLICATION

| | |
|---|---|
| Nucleic | Amino |
| Acid | Acid |

| | | | | |
|---|---|---|---|---|
| SEQ ID | SEQ ID | Identification | NT | NT |

| | | | | | | |
|---|---|---|---|---|---|---|
| NO | NO | Code | Contig. | Start | Stop | Function |

| | | | | | | |
|---|---|---|---|---|---|---|
| 1 | 2 | RXA00775 | GR00205 | 6057 | 5287 | PHOSPHATE TRANSPORT ATP-BINDING PROTEIN PSTB |
| 3 | 4 | RXA00776 | GR00205 | 7016 | 6096 | PHOSPHATE TRANSPORT SYSTEM PERMEASE PROTEIN PSTA |
| 5 | 6 | RXA00777 | GR00205 | 8098 | 7034 | PHOSPHATE TRANSPORT SYSTEM PERMEASE PROTEIN PSTC |
| 7 | 8 | RXA00774 | GR00205 | 4546 | 5199 | PHOSPHATE TRANSPORT SYSTEM REGULATORY PROTEIN |
| 9 | 10 | RXA00204 | GR00032 | 3783 | 2212 | RIBOSE TRANSPORT ATP-BINDING PROTEIN RBSA |
| 11 | 12 | RXA02438 | GR00709 | 3236 | 2478 | RIBOSE TRANSPORT ATP-BINDING PROTEIN RBSA |
| 13 | 14 | RXA00203 | GR00032 | 2152 | 1241 | RIBOSE TRANSPORT SYSTEM PERMEASE PROTEIN RBSC |
| 15 | 16 | RXA00270 | GR00041 | 2720 | 1833 | RIBOSE TRANSPORT SYSTEM PERMEASE PROTEIN RBSC |
| 17 | 18 | RXA02439 | GR00709 | 4258 | 3236 | RIBOSE TRANSPORT SYSTEM PERMEASE PROTEIN RBSC |
| 19 | 20 | RXN02994 | VV0070 | 2 | 724 | GLUTAMINE TRANSPORT ATP-BINDING PROTEIN GLNQ |
| 21 | 22 | F RXA01245 | GR00360 | 2 | 1768 | COPPER/POTASSIUM-TRANSPORTING ATPASE B (EC 3.6.1.36) |

Lipoprotein and Lipopolysaccharide synthesis

| | |
|---|---|
| Nucleic | Amino |
| Acid | Acid |

| | | | | |
|---|---|---|---|---|
| SEQ ID | SEQ ID | Identification | NT | NT |

| | | | | | | |
|---|---|---|---|---|---|---|
| NO | NO | Code | Contig. | Start | Stop | Function |

| | | | | | | |
|---|---|---|---|---|---|---|
| 23 | 24 | RXA00002 | GR00001 | 2278 | 1595 | DOLICHOL-PHOSPHATE MANNOSYLTRANSFERASE |
| | | | | | | (EC 2.4.1.83)/APOLIPOPROTEIN |
| | | | | | | N-ACYLTRANSFERASE (EC 2.3.1.-) |
| 25 | 26 | RXA00160 | GR00024 | 4044 | 4616 | LIPOPROTEIN NLPD/LPPB HOMOLOG PRECURSOR |
| 27 | 28 | RXA00345 | GR00064 | 90 | 1040 | Zn-binding lipoprotein |
| 29 | 30 | RXA00413 | GR00092 | 3859 | 2963 | OUTER MEMBRANE LIPOPROTEIN 3 PRECURSOR |
| 31 | 32 | RXA00482 | GR00119 | 18891 | 18244 | OUTER MEMBRANE LIPOPROTEIN BLC PRECURSOR |
| 33 | 34 | RXN01164 | VV0117 | 15894 | 14260 | DOLICHOL-PHOSPHATE MANNOSYLTRANSFERASE |
| | | | | | | (EC 2.4.1.83)/APOLIPOPROTEIN |
| | | | | | | N-ACYLTRANSFERASE (EC 2.3.1.-) |
| 35 | 36 | F RXA01164 | GR00332 | 1579 | 5 | DOLICHOL-PHOSPHATE MANNOSYLTRANSFERASE |
| | | | | | | (EC 2.4.1.83)/APOLIPOPROTEIN |
| | | | | | | N-ACYLTRANSFERASE (EC 2.3.1.-) |
| 37 | 38 | RXN01168 | VV0117 | 14224 | 13415 | DOLICHOL-PHOSPHATE MANNOSYLTRANSFERASE |
| | | | | | | (EC 2.4.1.83)/APOLIPOPROTEIN |
| | | | | | | N-ACYLTRANSFERASE (EC 2.3.1.-) |
| 39 | 40 | F RXA01168 | GR00333 | 1285 | 566 | DOLICHOL-PHOSPHATE MANNOSYLTRANSFERASE |
| | | | | | | (EC 2.4.1.83)/APOLIPOPROTEIN |
| | | | | | | N-ACYLTRANSFERASE (EC 2.3.1.-) |
| 41 | 42 | RXN02062 | VV0222 | 3159 | 1990 | Lipopolysaccharide N-acetylglucosaminyltransferase |
| 43 | 44 | F RXA02062 | GR00626 | 3159 | 1990 | Lipopolysaccharide N-acetylglucosaminyltransferase |
| 45 | 46 | RXA02222 | GR00651 | 9420 | 9794 | PUTATIVE HOST CELL SURFACE-EXPOSED LIPOPROTEIN |
| 47 | 48 | RXA02313 | GR00665 | 5812 | 4592 | Lipopolysaccharide N-acetylglucosaminyltransferase |
| 49 | 50 | RXA02491 | GR00720 | 902 | 2155 | Lipopolysaccharide N-acetylglucosaminyltransferase |
| 51 | 52 | RXN02595 | VV0098 | 11098 | 9935 | Lipopolysaccharide N-acetylglucosaminyltransferase |
| 53 | 54 | F RXA02595 | GR00741 | 19052 | 19702 | Lipopolysaccharide N-acetylglucosaminyltransferase |
| 55 | 56 | RXA02616 | GR00745 | 598 | 1308 | LIPOPROTEIN NLPD PRECURSOR |
| 57 | 58 | RXA02627 | GR00747 | 2981 | 2139 | DTXR/IRON-REGULATED LIPOPROTEIN PRECURSOR |
| 59 | 60 | RXA02650 | GR00752 | 1460 | 2038 | LIPOPROTEIN SIGNAL PEPTIDASE (EC 3.4.23.36) |
| 61 | 62 | RXA01094 | GR00306 | 2703 | 1756 | PROLIPOPROTEIN DIACYLGLYCERYL TRANSFERASE |
| | | | | | | (EC 2.4.99.-) |
| 63 | 64 | RXN00934 | VV0171 | 15181 | 14099 | (AE000805) LPS biosynthesis RfbU related protein |
| | | | | | | [Methanobacterium thermoautotrophicum] |
| 65 | 66 | F RXA00934 | GR00253 | 6835 | 6047 | (AE000805) LPS biosynthesis RfbU related protein |
| | | | | | | [Methanobacterium thermoautotrophicum] |
| 67 | 68 | RXA02605 | GR00742 | 11557 | 12051 | ANTIGEN 85-B PRECURSOR |

ABC-Transporter

| | |
|---|---|
| Nucleic | Amino |
| Acid | Acid |

| | | | | |
|---|---|---|---|---|
| SEQ ID | SEQ ID | Identification | NT | NT |

| | | | | | | |
|---|---|---|---|---|---|---|
| NO | NO | Code | Contig. | Start | Stop | Function |

| | | | | | | |
|---|---|---|---|---|---|---|
| 69 | 70 | RXN00525 | VV0079 | 26304 | 27566 | Hypothetical ABC Transporter Permease Protein |
| 71 | 72 | F RXA00525 | GR00136 | 664 | 5 | Hypothetical ABC Transporter Permease Protein |
| 73 | 74 | F RXA00556 | GR00146 | 1 | 594 | Hypothetical ABC Transporter Permease Protein |
| 75 | 76 | RXA02750 | GR00764 | 5079 | 5894 | Hypothetical ABC Transporter Permease Protein |
| 77 | 78 | RXN02096 | VV0126 | 20444 | 22135 | Hypothetical ABC Transporter Permease Protein |
| 79 | 80 | F RXA02096 | GR00629 | 15458 | 16774 | Hypothetical ABC Transporter Permease Protein |
| 81 | 82 | RXA02562 | GR00732 | 796 | 1515 | PUTATIVE ABC TRANSPORTER |
| 83 | 84 | RXA00950 | GR00260 | 173 | 1078 | similar to ABC transporter (ATP-binding protein) |
| | | | | | | START CODON GTG |
| 85 | 86 | RXA02119 | GR00636 | 4222 | 2582 | similar to ABC transporter (ATP-binding protein) |
| 87 | 88 | RXA01185 | GR00338 | 2451 | 1594 | ATP-BINDING PROTEIN |
| 89 | 90 | RXN00412 | VV0086 | 53923 | 52844 | Hypothetical Amino Acid ABC Transporter ATP-Binding Protein |
| 91 | 92 | F RXA00412 | GR00092 | 2764 | 1685 | ATP-BINDING PROTEIN ABC |
| 93 | 94 | RXN02925 | VV0104 | 543 | 2759 | COPPER/POTASSIUM-TRANSPORTING ATPASE B |
| | | | | | | (EC 3.6.1.36) |
| 95 | 96 | RXN00939 | VV0079 | 45152 | 43917 | COPPER/POTASSIUM-TRANSPORTING ATPASE B |
| | | | | | | (EC 3.6.1.36) |
| 97 | 98 | F RXA00939 | GR00256 | 1501 | 1334 | COPPER/POTASSIUM-TRANSPORTING ATPASE B |
| | | | | | | (EC 3.6.1.36) |
| 99 | 100 | RXN01323 | VV0082 | 4321 | 6585 | COPPER/POTASSIUM-TRANSPORTINGATPASE B |
| | | | | | | (EC 3.6.1.36) |
| 101 | 102 | F RXA01323 | GR00385 | 1175 | 3439 | similar to heavy metal-transporting ATPase |
| 103 | 104 | RXN00702 | VV0005 | 12478 | 10772 | COBALT TRANSPORT ATP-BINDING PROTEIN CBIO |
| 105 | 106 | F RXA00702 | GR00182 | 2165 | 846 | COBALT TRANSPORT ATP-BINDING PROTEIN CBIO |
| 107 | 108 | RXN00828 | VV0180 | 1376 | 1828 | COBALT TRANSPORT ATP-BINDING PROTEIN CBIO |
| 109 | 110 | F RXA00828 | GR00223 | 1687 | 1319 | COBALT TRANSPORT ATP-BINDING PROTEIN CBIO |
| 111 | 112 | RXN03020 | VV0139 | 606 | 4 | GLUTAMINE TRANSPORT ATP-BINDING PROTEIN GLNQ |
| 113 | 114 | RXN00726 | VV0188 | 1 | 591 | GLUTAMINE TRANSPORT ATP-BINDING PROTEIN GLNQ |
| 115 | 116 | RXN02570 | VV0101 | 11699 | 12340 | MALTOSE TRANSPORT SYSTEM PERMEASE PROTEIN MALF |
| 117 | 118 | RXN02354 | VV0095 | 473 | 1306 | MALTOSE TRANSPORT SYSTEM PERMEASE PROTEIN MALG |
| 119 | 120 | F RXA02354 | GR00682 | 473 | 1261 | MALTOSE TRANSPORT SYSTEM PERMEASE PROTEIN MALG |
| 121 | 122 | RXN00001 | VV0196 | 4023 | 2896 | MALTOSE/MALTODEXTRIN TRANSPORT ATP-BINDING PROTEIN |
| | | | | | | MALK |
| 123 | 124 | F RXA00001 | GR00001 | 1386 | 259 | SN-GLYCEROL-3-PHOSPHATE TRANSPORT ATP-BINDING |
| | | | | | | PROTEIN UGPC |
| 125 | 126 | RXN02356 | VV0051 | 1868 | 873 | MALTOSE/MALTODEXTRIN TRANSPORT ATP-BINDING PROTEIN |
| | | | | | | MALK |
| 127 | 128 | RXN02455 | VV0196 | 1273 | 5 | MALTOSE-BINDING PROTEIN PRECURSOR |
| 129 | 130 | RXN02795 | VV0176 | 29237 | 27801 | OLIGOPEPTIDE TRANSPORT ATP-BINDING PROTEIN APPF |
| 131 | 132 | F RXA02795 | GR00778 | 3 | 1097 | OLIGOPEPTIDE TRANSPORT ATP-BINDING PROTEIN APPF |
| 133 | 134 | RXN01939 | VV0139 | 22695 | 20965 | OLIGOPEPTIDE TRANSPORT ATP-BINDING PROTEIN OPPD |
| 135 | 136 | F RXA00761 | GR00203 | 8530 | 9120 | OLIGOPEPTIDE TRANSPORT ATP-BINDING PROTEIN OPPD |
| 137 | 138 | F RXA01939 | GR00556 | 2042 | 1440 | OLIGOPEPTIDE TRANSPORT ATP-BINDING PROTEIN OPPD |
| 139 | 140 | RXN00759 | VV0139 | 24645 | 23722 | OLIGOPEPTIDE TRANSPORT SYSTEM PERMEASE PROTEIN OPPB |
| 141 | 142 | F RXA00759 | GR00203 | 6580 | 7503 | OLIGOPEPTIDE TRANSPORT SYSTEM PERMEASE PROTEIN OPPB |
| 143 | 144 | RXN00431 | VV0112 | 8987 | 8199 | O-ANTIGEN EXPORT SYSTEM ATP-BINDING PROTEIN RFBE |
| 145 | 146 | F RXA00431 | GR00099 | 119 | 793 | ABCA PROTEIN two-component ABC transporter involved |
| | | | | | | in the metabolism of two wall teichoic acids |
| 147 | 148 | RXN00732 | VV0132 | 1 | 1647 | PROBABLE TRANSPORT ATP-BINDING PROTEIN MSBA |
| 149 | 150 | F RXA00732 | GR00196 | 826 | 5 | PROBABLE TRANSPORT ATP-BINDING PROTEIN MSBA |
| 151 | 152 | F RXA00734 | GR00197 | 863 | 411 | Hypothetical ABC Transporter ATP-Binding Protein |
| 153 | 154 | RXN01808 | VV0216 | 3 | 1151 | PUTATIVE ABC TRANPORTER |
| 155 | 156 | F RXA01808 | GR00509 | 8993 | 7875 | PUTATIVE ABC TRANPORTER |
| 157 | 158 | RXN02975 | VV0231 | 252 | 4 | Hypothetical ABC Transporter ATP-Binding Protein |
| 159 | 160 | RXN03116 | VV0090 | 38067 | 38675 | MALTOSE/MALTODEXTRIN TRANSPORT ATP-BINDING |
| | | | | | | PROTEIN MALK |
| 161 | 162 | RXN03108 | VV0077 | 5535 | 5801 | NITRATE TRANSPORT ATP-BINDING PROTEIN NRTD |
| 163 | 164 | RXN03129 | VV0122 | 24042 | 22819 | SN-GLYCEROL-3-PHOSPHATE TRANSPORT ATP-BINDING |
| | | | | | | PROTEIN UGPC |
| 165 | 166 | F RXA01890 | GR00541 | 874 | 155 | SN-GLYCEROL-3-PHOSPHATE TRANSPORT ATP-BINDING |
| | | | | | | PROTEIN UGPC |
| 167 | 168 | RXN02945 | VV0180 | 492 | 1424 | COBALT TRANSPORT ATP-BINDING PROTEIN CBIO |

Other transporters

| | |
|---|---|
| Nucleic | Amino |
| Acid | Acid |

| | | | | |
|---|---|---|---|---|
| SEQ ID | SEQ ID | Identification | NT | NT |

| | | | | | | |
|---|---|---|---|---|---|---|
| NO | NO | Code | Contig. | Start | Stop | Function |

| | | | | | | |
|---|---|---|---|---|---|---|
| 169 | 170 | RXA01247 | GR00361 | 256 | 489 | COPPER/POTASSIUM-TRANSPORTING ATPASE B (EC 3.6.1.36) |
| 171 | 172 | RXN00099 | VV0129 | 18876 | 17704 | CYANATE TRANSPORT PROTEIN CYNX |
| 173 | 174 | F RXA00099 | GR00014 | 8172 | 9344 | CYANATE TRANSPORT PROTEIN CYNX |
| 175 | 176 | RXA00634 | GR00166 | 3732 | 5114 | DI-/TRIPEPTIDE TRANSPORTER |
| 177 | 178 | RXA02451 | GR00710 | 3484 | 5007 | DI-/TRIPEPTIDE TRANSPORTER |
| 179 | 180 | RXA02394 | GR00697 | 1895 | 585 | DICARBOXYLATE TRANSPORTER |
| 181 | 182 | RXA01012 | GR00288 | 3748 | 2108 | DIPEPTIDE TRANSPORT ATP-BINDING PROTEIN DPPD |
| 183 | 184 | RXA02660 | GR00753 | 548 | 1186 | DIPEPTIDE TRANSPORT SYSTEM PERMEASE PROTEIN DPPB |
| 185 | 186 | RXA02661 | GR00753 | 1239 | 1457 | DIPEPTIDE TRANSPORT SYSTEM PERMEASE PROTEIN DPPB |
| 187 | 188 | RXA02034 | GR00619 | 1787 | 822 | DIPEPTIDE TRANSPORT SYSTEM PERMEASE PROTEIN DPPB |
| 189 | 190 | RXA01013 | GR00288 | 4549 | 3755 | DIPEPTIDE TRANSPORT SYSTEM PERMEASE PROTEIN DPPC |
| 191 | 192 | RXN02933 | VV0176 | 30042 | 29233 | DIPEPTIDE TRANSPORT SYSTEM PERMEASE PROTEIN DPPC |
| 193 | 194 | F RXA02033 | GR00619 | 800 | 12 | DIPEPTIDE TRANSPORT SYSTEM PERMEASE PROTEIN DPPC |
| 195 | 196 | RXA01006 | GR00287 | 862 | 5 | DIPEPTIDE TRANSPORTER PROTEIN DPPB |
| 197 | 198 | RXA02312 | GR00665 | 4459 | 3101 | D-SERINE/D-ALANINE/GLYCINE TRANSPORTER |
| 199 | 200 | RXA00090 | GR00013 | 6644 | 7762 | FERRIC ANGUIBACTIN TRANSPORT SYSTEM PERMEASE |
| | | | | | | PROTEIN FATC |
| 201 | 202 | RXA00089 | GR00013 | 5656 | 6654 | FERRIC ANGUIBACTIN TRANSPORT SYSTEM PERMEASE |
| | | | | | | PROTEIN FATD |
| 203 | 204 | RXN01285 | VV0215 | 1780 | 1055 | FERRIC ENTEROBACTIN TRANSPORT ATP-BINDING PROTEIN |
| | | | | | | FEPC |
| 205 | 206 | F RXA01285 | GR00371 | 3 | 545 | FERRIC ENTEROBACTIN TRANSPORT ATP-BINDING PROTEIN |
| | | | | | | FEPC |
| 207 | 208 | RXA02728 | GR00761 | 184 | 996 | FERRIC ENTEROBACTIN TRANSPORT ATP-BINDING PROTEIN |
| | | | | | | FEPC |
| 209 | 210 | RXN03080 | VV0045 | 1670 | 2449 | FERRIC ENTEROBACTIN TRANSPORT ATP-BINDING PROTEIN |
| | | | | | | FEPC |
| 211 | 212 | F RXA02864 | GR10007 | 2806 | 2027 | FERRIC ENTEROBACTIN TRANSPORT ATP-BINDING PROTEIN |
| | | | | | | FEPC |
| 213 | 214 | RXN00523 | VV0194 | 1363 | 338 | FERRIC ENTEROBACTIN TRANSPORT PROTEIN FEPG |
| 215 | 216 | F RXA00523 | GR00135 | 30 | 779 | FERRIC ENTEROBACTIN TRANSPORT PROTEIN FEPG |
| 217 | 218 | RXA01289 | GR00372 | 2376 | 3419 | FERRIC ENTEROBACTIN TRANSPORT PROTEIN FEPG |
| 219 | 220 | RXA01290 | GR00372 | 3412 | 4575 | FERRIC ENTEROBACTIN TRANSPORT PROTEIN FEPG |
| 221 | 222 | RXA01822 | GR00516 | 6 | 587 | FERRIC ENTEROBACTIN TRANSPORT PROTEIN FEPG |
| 223 | 224 | RXN00466 | VV0086 | 63271 | 64266 | Ferrichrome transport proteins |
| 225 | 226 | F RXA00466 | GR00117 | 947 | 1933 | Ferrichrome transport proteins |
| 227 | 228 | RXN03081 | VV0045 | 2476 | 2934 | FERRIENTEROBACTIN-BINDING PERIPLASMIC PROTEIN |
| | | | | | | PRECURSOR |
| 229 | 230 | F RXA02863 | GR10007 | 2000 | 1026 | Ferrichrome transport proteins |
| 231 | 232 | RXS03221 | | | | GALACTOSE-PROTON SYMPORT |
| 233 | 234 | F RXA01986 | GR00575 | 622 | 5 | GALACTOSE-PROTON SYMPORT |
| 235 | 236 | RXN02447 | VV0107 | 14297 | 13203 | GALACTOSE-PROTON SYMPORT |
| 237 | 238 | F RXA02447 | GR00710 | 1 | 270 | GALACTOSE-PROTON SYMPORT |
| 239 | 240 | F RXA02769 | GR00771 | 1 | 711 | GALACTOSE-PROTON SYMPORT |
| 241 | 242 | RXS503220 | | | | D-XYLOSE-PROTON SYMPORT |
| 243 | 244 | F RXA02762 | GR00768 | 346 | 630 | D-XYLOSE PROTON-SYMPORTER |
| 245 | 246 | F RXA02761 | GR00768 | 153 | 353 | GALACTOSE-PROTON SYMPORT |
| 247 | 248 | RXA00123 | GR00019 | 7029 | 5911 | MAGNESIUM AND COBALT TRANSPORT PROTEIN CORA |
| 249 | 250 | RXA02441 | GR00709 | 5940 | 5284 | MANGANESE TRANSPORT SYSTEM ATP-BINDING PROTEIN MNTA |
| 251 | 252 | RXN02442 | VV0217 | 5970 | 6818 | zinc transport system membrane protein |
| 253 | 254 | F RXA02442 | GR00709 | 5970 | 6818 | MANGANESE TRANSPORT SYSTEM MEMBRANE PROTEIN MNTB |
| 255 | 256 | RXA01756 | GR00498 | 2069 | 762 | MG2+ TRANSPORTER MGTE |
| 257 | 258 | RXA02068 | GR00627 | 2 | 1120 | MG2+ TRANSPORTER MGTE |
| 259 | 260 | RXA00665 | GR00174 | 135 | 572 | MG2+/CITRATE COMPLEX SECONDARY TRANSPORTER |
| 261 | 262 | RXA02808 | GR00789 | 1 | 258 | MG2+/CITRATE COMPLEX SECONDARY TRANSPORTER |
| 263 | 264 | RXN00444 | VV0112 | 20785 | 19949 | MOLYBDENUM TRANSPORT SYSTEM PERMEASE PROTEIN MODB |
| 265 | 266 | F RXA00444 | GR00106 | 626 | 1402 | MOLYBDENUM TRANSPORT SYSTEM PERMEASE PROTEIN MODB |
| 267 | 268 | RXN02614 | VV0313 | 5964 | 5236 | TAURINE TRANSPORT ATP-BINDING PROTEIN TAUB |
| 269 | 270 | F RXA02614 | GR00743 | 5964 | 5236 | NITRATE TRANSPORT ATP-BINDING PROTEIN NRTC |
| 271 | 272 | RXN01142 | VV0077 | 5805 | 6302 | NITRATE TRANSPORT ATP-BINDING PROTEIN NRTD |
| 273 | 274 | F RXA01142 | GR00320 | 721 | 302 | NITRATE TRANSPORT ATP-BINDING PROTEIN NRTD |
| 275 | 276 | RXN01141 | VV0077 | 4644 | 5468 | NITRATE TRANSPORT PROTEIN NRTA |
| 277 | 278 | F RXA01135 | GR00318 | 327 | 4 | NITRATE TRANSPORT PROTEIN NRTA |
| 279 | 280 | F RXA01141 | GR00319 | 636 | 175 | NITRATE TRANSPORT PROTEIN NRTA |
| 281 | 282 | RXA00728 | GR00193 | 1658 | 2449 | NOPALINE TRANSPORT SYSTEM PERMEASE PROTEIN NOCM |
| 283 | 284 | RXA02663 | GR00753 | 2059 | 3453 | OLIGOPEPTIDE TRANSPORT ATP-BINDING PROTEIN OPPD |
| 285 | 286 | RXA02664 | GR00753 | 3611 | 4270 | OLIGOPEPTIDE TRANSPORT ATP-BINDING PROTEIN OPPF |
| 287 | 288 | RXA00760 | GR00203 | 7499 | 8530 | OLIGOPEPTIDE TRANSPORT SYSTEM PERMEASE PROTEIN OPPC |
| 289 | 290 | RXA02035 | GR00619 | 3295 | 1787 | PERIPLASMIC DIPEPTIDE TRANSPORT PROTEIN PRECURSOR |
| 291 | 292 | RXN01002 | VV0106 | 8858 | 8055 | PHOSPHONATES TRANSPORT ATP-BINDING PROTEIN PHNC |
| 293 | 294 | F RXA01002 | GR00285 | 3 | 419 | PHOSPHONATES TRANSPORT ATP-BINDING PROTEIN PHNC |
| 295 | 296 | RXN01000 | VV0106 | 7252 | 6407 | PHOSPHONATES TRANSPORT SYSTEM PERMEASE PROTEIN PHNE |
| 297 | 298 | F RXA01000 | GR00284 | 2 | 541 | PHOSPHONATES TRANSPORT SYSTEM PERMEASE PROTEIN PHNE |
| 299 | 300 | RXA01003 | GR00285 | 419 | 1222 | PHOSPHONATES TRANSPORT SYSTEM PERMEASE PROTEIN PHNE |
| 301 | 302 | RXN00193 | VV0371 | 1 | 594 | POTENTIAL STARCH DEGRADATION PRODUCTS TRANSPORT |
| | | | | | | SYSTEM PERMEASE AMYD |
| 303 | 304 | F RXA00193 | GR00029 | 10101 | 9259 | POTENTIAL STARCH DEGRADATION PRODUCTS TRANSPORT |
| | | | | | | SYSTEM PERMEASE AMYD |
| 305 | 306 | RXN01298 | VV0116 | 2071 | 1142 | POTENTIAL STARCH DEGRADATION PRODUCTS TRANSPORT |
| | | | | | | SYSTEM PERMEASE PROTEIN AMYD |
| 307 | 308 | F RXA01298 | GR00374 | 1254 | 862 | POTENTIAL STARCH DEGRADATION PRODUCTS TRANSPORT |
| | | | | | | SYSTEM PERMEASE PROTEIN AMYD |
| 309 | 310 | F RXA02422 | GR00705 | 8200 | 8634 | POTENTIAL STARCH DEGRADATION PRODUCTS TRANSPORT |
| | | | | | | SYSTEM PERMEASE PROTEIN AMYD |
| 311 | 312 | RXN02515 | VV0087 | 962 | 1717 | Hypothetical ABC Transporter ATP-Binding Protein |
| 313 | 314 | F RXA02515 | GR00723 | 964 | 1719 | PROBABLE ATP-DEPENDENT TRANSPORTER YCF16 |
| 315 | 316 | RXN01995 | VV0182 | 2139 | 3476 | PUTATIVE 3-(3-HYDROXYPHENYL) PROPIONATE TRANSPORT |
| | | | | | | PROTEIN |
| 317 | 318 | F RXA01995 | GR00584 | 1362 | 2015 | PUTATIVE 3-(3-HYDROXYPHENYL) PROPIONATE TRANSPORT |
| | | | | | | PROTEIN |
| 319 | 320 | RXA01188 | GR00339 | 1585 | 482 | PUTATIVE TRANSPORT PROTEIN SGAT |
| 321 | 322 | RXA01972 | GR00569 | 2116 | 1523 | QUATERNARY AMINE TRANSPORTER |
| 323 | 324 | RXA00311 | GR00053 | 1592 | 738 | SHIKIMATE TRANSPORTER |
| 325 | 326 | RXA00312 | GR00053 | 2066 | 1641 | SHIKIMATE TRANSPORTER |
| 327 | 328 | RXN01411 | VV0050 | 26015 | 26779 | SHIKIMATE TRANSPORTER |
| 329 | 330 | F RXA01411 | GR00412 | 1 | 327 | SHIKIMATE TRANSPORTER |
| 331 | 332 | RXA01900 | GR00544 | 2822 | 4120 | SHIKIMATE TRANSPORTER |
| 333 | 334 | RXA02507 | GR00720 | 19760 | 21160 | SHIKIMATE TRANSPORTER |
| 335 | 336 | RXA00445 | GR00107 | 21 | 932 | SN-GLYCEROL-3-PHOSPHATE TRANSPORT ATP-BINDING PROTEIN |
| | | | | | | UGPC |
| 337 | 338 | RXA02353 | GR00682 | 6 | 473 | SN-GLYCEROL-3-PHOSPHATE TRANSPORT SYSTEM PERMASE |
| | | | | | | PROTEIN UGPA |
| 339 | 340 | RXA01297 | GR00374 | 826 | 29 | SN-GLYCEROL-3-PHOSPHATE TRANSPORT SYSTEM PERMEASE |
| | | | | | | PROTEIN |
| 341 | 342 | RXS00088 | VV0027 | 2 | 877 | FERRIC ANGUIBACTIN-BINDING PROTEIN PRECURSOR |
| 343 | 344 | RXS00372 | VV0226 | 3456 | 2380 | PERIPLASMIC-IRON-BINDING PROTEIN SHIB |
| 345 | 346 | RXS02590 | VV0098 | 15313 | 16248 | MALIC ACID TRANSPORT PROTEIN |
| 347 | 348 | RXS00758 | VV0139 | 26428 | 24827 | PERIPLASMIC OLIGOPEPTIDE-BINDING PROTEIN PRECURSOR |
| 349 | 350 | RXS01346 | VV0123 | 5120 | 6694 | PERIPLASMIC OLIGOPEPTIDE-BINDING PROTEIN PRECURSOR |
| 351 | 352 | RXS00912 | VV0339 | 552 | 280 | potassium efflux system protein phaF |
| 353 | 354 | RXS00453 | VV0076 | 1173 | 3521 | Drug Transporter |
| 355 | 356 | RXS00932 | VV0171 | 13120 | 13593 | Drug Transporter |
| 357 | 358 | RXS00479 | VV0086 | 42008 | 39819 | Drug Transporter |
| 359 | 360 | RXS02586 | VV0098 | 19854 | 20123 | Drug Transporter |
| 361 | 362 | RXS02587 | VV0098 | 17807 | 19897 | Drug Transporter |
| 363 | 364 | RXS03042 | VV0018 | 2440 | 1835 | Drug Transporter |
| 365 | 366 | RXS03075 | VV0042 | 2491 | 3216 | Drug Transporter |
| 367 | 368 | RXS03124 | VV0108 | 4 | 963 | Drug Transporter |
| 369 | 370 | RXS03125 | VV0108 | 972 | 1142 | Drug Transporter |

Channel Proteins

| | |
|---|---|
| Nucleic | Amino |
| Acid | Acid |

| | | | | |
|---|---|---|---|---|
| SEQ ID | SEQ ID | Identification | NT | NT |

| | | | | | | |
|---|---|---|---|---|---|---|
| NO | NO | Code | Contig. | Start | Stop | Function |

| | | | | | | |
|---|---|---|---|---|---|---|
| 371 | 372 | RXA00596 | GR00159 | 335 | 787 | potassium efflux system protein phaE |
| 373 | 374 | RXA02079 | GR00628 | 9034 | 9648 | CATION EFFLUX SYSTEM PROTEIN CZCD |
| 375 | 376 | RXA01303 | GR00376 | 1724 | 390 | NITRITE EXTRUSION PROTEIN |
| 377 | 378 | RXA02079 | GR00628 | 9034 | 9648 | CATION EFFLUX SYSTEM PROTEIN CZCD |
| 379 | 380 | RXN00832 | VV0180 | 3133 | 4182 | CALCIUM/PROTON ANTIPORTER |
| 381 | 382 | F RXA00832 | GR00224 | 2239 | 1685 | CALCIUM/PROTON ANTIPORTER |
| 383 | 384 | RXN00378 | VV0223 | 8027 | 5418 | Cation transport ATPases |
| 385 | 386 | F RXA00378 | GR00081 | 3271 | 1499 | Cation transport ATPases |
| 387 | 388 | RXA00942 | GR00257 | 2406 | 2203 | CATION-TRANSPORTING ATPASE PACS (EC 3.6.1.-) |
| 389 | 390 | RXN01338 | VV0032 | 2 | 1903 | CATION-TRANSPORTING ATPASE PACS (EC 3.6.1.-) |
| 391 | 392 | F RXA01338 | GR00389 | 6964 | 5087 | CATION-TRANSPORTING ATPASE PACS (EC 3.6.1.-) |
| 393 | 394 | RXA01625 | GR00452 | 3850 | 3650 | CATION-TRANSPORTING ATPASE PACS (EC 3.6.1.-) |
| 395 | 396 | RXA02220 | GR00651 | 3205 | 5880 | CATION-TRANSPORTING ATPASE PMA1 (EC 3.6.1.-) |
| 397 | 398 | RXN00980 | VV0149 | 2635 | 4428 | CATION-TRANSPORTING P-TYPE ATPASE B (EC 3.6.1.-) |
| 399 | 400 | F RXA00980 | GR00276 | 2648 | 3286 | CATION-TRANSPORTING P-TYPE ATPASE B (EC 3.6.1.-) |
| 401 | 402 | RXN02348 | VV0078 | 6027 | 7910 | KUP SYSTEM POTASSIUM UPTAKE PROTEIN |
| 403 | 404 | F RXA02348 | GR00677 | 1719 | 586 | KUP SYSTEM POTASSIUM UPTAKE PROTEIN |
| 405 | 406 | F RXA02344 | GR00676 | 682 | 5 | KUP SYSTEM POTASSIUM UPTAKE PROTEIN |
| 407 | 408 | RXN00960 | VV0075 | 1139 | 105 | PROTON/SODIUM-GLUTAMATE SYMPORT PROTEIN |
| 409 | 410 | F RXA00960 | GR00266 | 563 | 105 | PROTON/SODIUM-GLUTAMATE SYMPORT PROTEIN |
| 411 | 412 | RXA01070 | GR00299 | 2089 | 704 | PROTON/SODIUM-GLUTAMATE SYMPORT PROTEIN |
| 413 | 414 | RXA02628 | GR00748 | 6 | 410 | LARGE CONDUCTANCE MECHANOSENSITIVE CHANNEL |
| 415 | 416 | RXN03164 | VV0277 | 1586 | 2455 | POTASSIUM CHANNEL BETA SUBUNIT |
| 417 | 418 | F RXA01395 | GR00408 | 6106 | 5021 | POTASSIUM CHANNEL BETA SUBUNIT |

Other membrane proteins

| | |
|---|---|
| Nucleic | Amino |
| Acid | Acid |

| | | | | |
|---|---|---|---|---|
| SEQ ID | SEQ ID | Identification | NT | NT |

| | | | | | | |
|---|---|---|---|---|---|---|
| NO | NO | Code | Contig. | Start | Stop | Function |

| | | | | | | |
|---|---|---|---|---|---|---|
| 419 | 420 | RXA02597 | GR00742 | 2329 | 542 | OUTER MEMBRANE USHER PROTEIN FIMC PRECURSOR |
| 421 | 422 | RXA01454 | GR00420 | 270 | 4 | integral membrane protein |
| 423 | 424 | RXA01455 | GR00420 | 745 | 284 | integral membrane protein |
| 425 | 426 | RXA02684 | GR00754 | 8923 | 8060 | MEMBRANE-BOUND PROTEIN LYTR |
| 427 | 428 | RXN02391 | VV0176 | 3525 | 3923 | (U59457 Pseudomonas aeruginosa ankyrin (ankB) gene, |
| | | | | | | complete cds [Pseudomonas aeruginosa] |
| 429 | 430 | RXN02549 | VV0098 | 3165 | 5867 | PUTATIVE INTEGRAL MEMBRANE PROTEIN |
| 431 | 432 | RXN00808 | VV0009 | 63243 | 64700 | PUTATIVE MEMBRANE PROTEIN |
| 433 | 434 | RXS01425 | VV0050 | 2679 | 3563 | 60 KD INNER-MEMBRANE PROTEIN |
| 435 | 436 | RXS01658 | VV0010 | 44183 | 42351 | membrane protein |
| 437 | 438 | RXS01677 | VV0179 | 12923 | 12180 | membrane protein |
| 439 | 440 | RXS02932 | VV0176 | 23391 | 24362 | Membrane Spanning Protein |
| 441 | 442 | F RXA02402 | GR00700 | 747 | 4 | (AF027868) putative transporter [Bacillius subtilis] |
| 443 | 444 | RXS00654 | VV0109 | 6289 | 5024 | 6O KD INNER-MEMBRANE PROTEIN |

1. An isolated nucleic acid molecule selected from the group consisting of
a) an isolated nucleic acid molecule from Corynebacterium glutamicum encoding an MCT protein, provided that the nucleic acid molecule does not consist of any of the F-designated genes set forth in Table 1, or a complement thereof;
b) an isolated Corynebacterium glutamicum nucleic acid molecule comprising a nucleotide sequence selected from the group consisting of those sequences set forth in Appendix A, provided that the nucleic acid molecule does not consist of any of the F-designated genes set forth in Table 1, or a complement thereof;
c) an isolated nucleic acid molecule which encodes a polypeptide comprising an amino acid sequence selected from the group consisting of those sequences set forth in Appendix B, provided that the nucleic acid molecule does not consist of any of the F-designated genes set forth in Table 1, or a complement thereof;
d) an isolated nucleic acid molecule which encodes a naturally occurring allelic variant of a polypeptide comprising an amino acid sequence selected from the group consisting of those sequences set forth in Appendix B, provided that the nucleic acid molecule does not consist of any of the F-designated genes set forth in Table 1, or a complement thereof;
e) an isolated nucleic acid molecule comprising a nucleotide sequence which is at least 50% identical to an entire nucleotide sequence selected from the group consisting of those sequences set forth in Appendix A, provided that the nucleic acid molecule does not consist of any of the F-designated genes set forth in Table 1, or a complement thereof; and
f) an isolated nucleic acid molecule comprising a fragment of at least 15 contiguous nucleotides of a nucleotide sequence selected from the group consisting of those sequences set forth in Appendix A, provided that the nucleic acid molecule does not consist of any of the F-designated genes set forth in Table 1, or a complement thereof.

2. The isolated nucleic acid molecule of claim 1, wherein said nucleic acid molecule from Corynebacterium glutamicum encodes an MCT protein involved in the production of a fine chemical.

3. An isolated nucleic acid molecule comprising the nucleic acid molecule of claim 1 and a nucleotide sequence encoding a heterologous polypeptide.

4. A vector comprising the nucleic acid molecule of claim 1.

5. The vector of claim 4, which is an expression vector.

6. A host cell transfected with the expression vector of claim 5.

7. The host cell of claim 6, wherein said cell is a microorganism.

8. The host cell of claim 7, wherein said cell belongs to the genus Corynebacterium or Brevibacterium.

9. A method of producing a polypeptide comprising culturing the host cell of claim 6 in an appropriate culture medium to, thereby, produce the polypeptide.

10. A method for producing a fine chemical, comprising culturing the cell of claim 6 such that the fine chemical is produced.

11. The method of claim 10, wherein said method further comprises the step of recovering the fine chemical from said culture.

12. The method of claim 10, wherein said cell belongs to the genus Corynebacterium or Brevibacterium.

13. The method of claim 10, wherein said cell is selected from the group consisting of Corynebacterium glutamicum, Corynebacterium herculis, Corynebacterium, lilium, Corynebacterium acetoacidophilum, Corynebacterium acetoglutamicum, Corynebacterium acetophilum, Corynebacterium ammoniagenes, Corynebacterium fujiokense, Corynebacterium nitrilophilus, Brevibacterium ammoniagenes, Brevibacterium butanicum, Brevibacterium divaricatum, Brevibacterium flavum, Brevibacterium healii, Brevibacterium ketoglutamicum, Brevibacterium ketosoreductum, Brevibacterium lactofermentum, Brevibacterium linens, Brevibacterium paraffinolyticum, and those strains set forth in Table 3.

14. The method of claim 10, wherein expression of the nucleic acid molecule from said vector results in modulation of production of said fine chemical.

15. The method of claim 10, wherein said fine chemical is selected from the group consisting of organic acids, proteinogenic and nonproteinogenic amino acids, purine and pyrimidine bases, nucleosides, nucleotides, lipids, saturated and unsaturated fatty acids, diols, carbohydrates, aromatic compounds, vitamins, cofactors, polyketides, and enzymes.

16. The method of claim 10, wherein said fine chemical is an amino acid selected from the group consisting of lysine, glutamate, glutamine, alanine, aspartate, glycine, serine, threonine, methionine, cysteine, valine, leucine, isoleucine, arginine, proline, histidine, tyrosine, phenylalanine, and tryptophan.

17. An isolated polypeptide selected from the group consisting of
a) an isolated MCT polypeptide from Corynebacterium glutamicum;
b) an isolated polypeptide comprising an amino acid sequence selected from the group consisting of those sequences set forth in Appendix B, provided that the amino acid sequence is not encoded by any of the F-designated genes set forth in Table 1;
c) an isolated polypeptide comprising a naturally occurring allelic variant of a polypeptide comprising an amino acid sequence selected from the group consisting of those sequences set forth in Appendix B, provided that the amino acid sequence is not encoded by any of the F-designated genes set forth in Table 1;
d) an isolated polypeptide which is encoded by a nucleic acid molecule comprising a nucleotide sequence which is at least 50% identical to an entire nucleotide sequence selected from the group consisting of those sequences set forth in Appendix A, provided that the nucleic acid molecule does not consist of any of the F-designated genes set forth in Table 1;
e) an isolated polypeptide comprising an amino acid sequence which is at least 50% identical to an entire amino acid sequence selected from the group consisting of those sequences set forth in Appendix B, provided that the amino acid sequence is not encoded by any of the F-designated genes set forth in Table 1; and
f) an isolated polypeptide comprising a fragment of a polypeptide comprising an amino acid sequence selected from the group consisting of those sequences set forth in Appendix B, provided that the amino acid sequence is not encoded by any of the F-designated genes set forth in Table 1, wherein said polypeptide fragment maintains a biological activity of the polypeptide comprising the amino sequence.

18. The isolated polypeptide of claim 17, wherein said polypeptide is involved in the production of a fine chemical.

19. The isolated polypeptide of claim 17, further comprising heterologous amino acid sequences.

20. A method for diagnosing the presence or activity of Corynebacterium diphtheriae in a subject, comprising detecting the presence of at least one of the nucleic acid molecules of claim 1, thereby diagnosing the presence or activity of Corynebacterium diphtheriae in the subject.

21. A method for diagnosing the presence or activity of Corynebacterium diphtheriae in a subject, comprising detecting the presence of at least one of the polypeptide molecules of claim 17, thereby diagnosing the presence or activity of Corynebacterium diphtheriae in the subject.

22. A host cell comprising a nucleic acid molecule selected from the group consisting of
a) a nucleic acid molecule set forth in Appendix A, wherein the nucleic acid molecule is disrupted by at least one technique selected from the group consisting of a point mutation, a truncation, an inversion, a deletion, an addition, a substitution and homologous recombination;
b) a nucleic acid molecule set forth in Appendix A, wherein the nucleic acid molecule comprises one or more nucleic acid modifications as compared to the sequence set forth in Appendix A, wherein the modification is selected from the group consisting of a point mutation, a truncation, an inversion, a deletion, an addition and a substitution; and
c) a nucleic acid molecule set forth in Appendix A, wherein the regulatory region of the nucleic acid molecule is modified relative to the wild-type regulatory region of the molecule by at least one technique selected from the group consisting of a point mutation, a truncation, an inversion, a deletion, an addition, a substitution and homologous recombination.
